(19) United States
(12) Patent Application Publication
(10) Pub. No.: US 2021/0054405 A1
(43) Pub. Date: Feb. 25, 2021

(54) CLOSED-ENDED DNA (CEDNA) VECTORS FOR INSERTION OF TRANSGENES AT GENOMIC SAFE HARBORS (GSH) IN HUMANS AND MURINE GENOMES

(71) Applicant: Generation Bio Co., Cambridge, MA (US)

(72) Inventor: Robert M. Kotin, Cambridge, MA (US)

(21) Appl. No.: 16/977,506

(22) PCT Filed: Mar. 1, 2019
(86) PCT No.: PCT/US2019/020225

§ 371 Date: Sep. 2, 2020

Related U.S. Application Data
(60) Provisional application No. 62/637,594, filed on Mar. 2, 2018.
(60) Provisional application No. 62/716,431, filed on Aug. 9, 2018.

(51) Int. Cl. C12N 15/85; C12N 15/90

(52) U.S. Cl. CPC C12N 15/85; C12N 15/907; C12N 2800/107; C12N 2710/14043; C12N 15/8509

(57) ABSTRACT

The application describes ceDNA vectors having linear and continuous structure for insertion of a transgene into a gene safe harbor (GSH) in a genome, e.g., mammalian genome. ceDNA vectors can comprise at least one ITR sequence, or two ITR sequences, a transgene, and at least one nucleic acid sequence that specifically binds to, or hybridizes to a GSH locus. Some ceDNA vectors comprise at least one GSH homology arm (GSH HA), e.g., a 5′ GSH HA, and/or a 3′ GSH HA, and some ceDNA vectors comprise a guide RNA (gRNA) or guide DNA (gDNA) that specifically targets a region in the GSH locus and/or a 5′ or 3′ GSH HA herein. Some ceDNA vectors also comprise a gene editing cassette that encodes a gene editing molecule. Some ceDNA vectors further comprise cis-regulatory elements, including regulatory switches for regulation of the transgene expression after its insertion at a GSH

### CROSS REFERENCE TO RELATED APPLICATIONS

This application claims benefit under 35 U.S.C. § 119(e) of U.S. Provisional Application Nos. 62/637,594, filed Mar. 2, 2018 and 62/716,431, filed on Aug. 9, 2018, the content of each of which is incorporated herein by reference in its entirety.

### SEQUENCE LISTING

The instant application contains a Sequence Listing which has been submitted electronically in ASCII format and is hereby incorporated by reference in its entirety. Said ASCII copy, created on Feb. 28, 2019, is named 080170-090750WOPT_SL.txt and is 116,841 bytes in size.

### TECHNICAL FIELD

The present disclosure relates to the field of gene therapy, including identification, characterizing and validating genomic safe harbor (GSH) locus in mammalian, including human genomes. The disclosure relates to a method to identify the GSH, and methods to validate the GSH using ceDNA vectors, and recombinant nucleic acid ceDNA vectors comprising nucleic acids complementary to regions of the GSH that guides homologous recombination with regions of the GSH, as well as cells, kits and transgenic animals comprising the ceDNA vectors, and/or transgenes inserted at a GSH using a ceDNA vector.

### BACKGROUND

The modification of the human genome by the stable insertion of functional transgenes and other genetic elements is of great value in biomedical research and medicine. Several diseases have now been successfully treated with gene therapy. Genetically modified human cells are also valuable for the study of gene function, and for tracking and lineage analyses using reporter systems. All these applications depend on the reliable function of the introduced genes in their new environments. However, randomly inserted genes are subject to position effects and silencing, making their expression unreliable and unpredictable. Centromeres and sub-telomeric regions are particularly prone to transgene silencing. Reciprocally, newly integrated genes may affect the surrounding endogenous genes and chromatin, potentially altering cell behavior or favoring cellular transformation. Despite the successes of therapeutic gene transfer, there have been several cases of malignant transformation associated with insertional activation of oncogenes following stem cell gene therapy, emphasizing the importance of where newly integrated DNA locates.

Despite this, the gene editing field has evolved from classical but inefficient homologous recombination, to more specific and efficient DNA nuclease mediated recombination using zinc finger nuclease and TALENS, to widely used CRISPR/Cas9 nuclease technology. Because of the robustness of the CRISPR/Cas9 methodologies, gene editing has become routine for non-specialized research groups. However, the insertion of foreign DNA into the genome of progenitor cells may adversely affect terminal differentiation into specific cell types. A genomic safe harbor (GSH) refers to a genetic locus that accommodates the insertion of exogenous DNA with either constitutive or conditional expression activity without significantly affecting the viability of somatic cells, progenitor cells, or germ line cells and ontogeny.

The availability of such GSH loci would be extremely useful to express reporter genes, suicide genes, selectable genes or therapeutic genes. Three intragenic sites have been proposed as GSHs (AAVS1, CCR5 and ROSA26 and albumin in murine cells) (see, e.g., U.S. Pat. Nos. 7,951,925; 8,771,985; 8,110,379; 7,951,925; U.S. Publication Nos. 20100218264; 20110265198; 20130137104; 20130122591; 20130177983; 20130177960; 20150056705 and 20150159172). However, these proposed GSHs are in relatively gene-rich regions and are near genes that have been implicated in cancer. Genes that are adjacent to AAVS1 may be spared by some promoters, but safety validation in multiple tissues remains to be carried out. Also, the dispensability of the disrupted gene, especially after biallelic disruption, as is often the case with endonuclease-mediated targeting, remains to be investigated further.

Therefore, the identification of more sites would be highly valuable, especially at extragenic or intergenic regions. There is also a need to identify, qualify and validate candidate GSH loci for research and potential therapeutic applications, in particular, because transgene expression may vary by GSH loci, developmental stage, and tissue type. In addition, the targeted cell “potency” may be affected in a GSH-dependent manner, for example, hematopoietic stem cells (HSC) and embryonic stem cells (ESC). Therefore, identifying multiple GSH loci in the human and mouse genomes may provide a catalog of sites for different applications, including e.g., expression of a nucleic acid of interest, such as, e.g., therapeutic RNA, miRNAs, therapeutic proteins and nucleic acids, and suicide genes and the like.

### SUMMARY

The disclosure herein relates to a non-viral, capsid-free DNA vector with covalently-closed ends (referred to herein as a “closed-ended DNA vector” or a “ceDNA vector”) for insertion of a transgene into specific genomic safe harbor (GSH) regions, and methods of use of such ceDNA vectors, e.g., to treat a disease.

In some embodiments, a ceDNA vector as described herein are capsid-free, linear duplex DNA molecules formed from a continuous strand of complementary DNA with covalently-closed ends (linear, continuous and non-encapsulated structure), which comprises at least one ITR sequence, or at least two inverted terminal repeat (ITR) sequences flanking a nucleic acid construct, the nucleic acid construct comprising a at least one Gene Safe Harbor (GSH) homology arm (referred to herein as a GSH HA), such as a left GSH homology arm (also referred to as a GSH HA-L or 5′ GSH HA), a heterologous nucleic acid construct comprising at least one gene of interest (GOI) (or transgene), and a right GSH homology arm (also referred to as a GSH HA-R or 3′ GSH HA). In some embodiments, the GOI can be genomic DNA (gDNA) encoding a protein or nucleic acid of interest, where the GOI has an open reading frame (ORF) and comprises introns and exons, or alternatively, the GOI can be complementary DNA (cDNA) i.e., lacking introns). In some embodiments, the GOI can be operatively linked to any one or more of: a promoter or regulatory switch as defined herein, a 5′ UTR, a 3′ UTR, a polyadenylation sequence, post-transcriptional elements which is operatively linked to a promoter or other regulatory switch as described herein. An exemplary ceDNA vector for insertion of a GOI into a GSH as described herein is shown in FIG. 1A. This embodiment shows two ITRs flanking the 5′ GSH HA and a 3′ GSH, however, it is envisioned that only one ITR can be used, and/or one GSH homology arm can be used, e.g., see FIGS. 9B, 9C. In embodiments where there are two ITRs, the 5′ ITR and the 3′ ITR of a ceDNA vector as disclosed herein can have the same symmetrical three-dimensional organization with respect to each other, (i.e., symmetrical or substantially symmetrical), or alternatively, the 5′ ITR and the 3′ ITR can have different three-dimensional organization with respect to each other (i.e., asymmetrical ITRs), as these terms are defined herein. In addition, the ITRs can be from the same or different serotypes. In some embodiments, a ceDNA vector can comprise ITR sequences that have a symmetrical three-dimensional spatial organization such that their structure is the same shape in geometrical space, or have the same A, C-C′ and B-B′ loops in 3D space (i.e., they are the same or are mirror images with respect to each other). In some embodiments, one ITR can be from one AAV serotype, and the other ITR can be from a different AAV serotype.

In some embodiments, a ceDNA vector described herein for integration of a nucleic acid of interest into a GSH locus can comprise: a first ITR, a 5′ GSH specific HA (HA-L), a nucleic acid of interest and/or an expressible transgene cassette (e.g., a sequence that encodes a therapeutic protein or nucleic acid as described herein, and/or a reporter protein), and/or a 3′GSH HA (HA-R), and a second ITR. For example, in some embodiments, a ceDNA vector can comprise: a first ITR, a 5′ GSH specific HA (HA-L), a nucleic acid of interest and/or an expressible transgene cassette (e.g., a sequence that encodes a therapeutic protein or nucleic acid as described herein, and/or a reporter protein), and a 3′GSH HA (HA-R), and a second ITR. In alternative embodiments, a ceDNA vector can comprise: a first ITR, a 5′ GSH specific HA (HA-L), a nucleic acid of interest and/or an expressible transgene cassette (e.g., a sequence that encodes a therapeutic protein or nucleic acid as described herein, and/or a reporter protein), and a second ITR. In alternative embodiments, a ceDNA vector can comprise: a first ITR, a nucleic acid of interest and/or an expressible transgene cassette (e.g., a sequence that encodes a therapeutic protein or nucleic acid as described herein, and/or a reporter protein), and a 3′GSH HA (HA-R), and a second ITR. In some embodiments, such ceDNA vectors comprise a first ITR only (e.g., a 5′ ITR but do not comprise a 3′ ITR). In alternative embodiments, such ceDNA vectors can comprise a second ITR only (e.g., a 3′ ITR) and not a 5′ ITR. In some embodiments, such ceDNA vectors can also comprise a gene editing cassette as described herein, e.g., located 3′ of the 5′ ITR (first ITR), but 5′ of the 5′ homology arm. In alternative embodiments, a ceDNA vector can also comprise a gene editing cassette as described herein, e.g, located 5′ of the 3′ ITR (second ITR), but 3′ of the 3′ homology arm. In some embodiments, where the gene editing cassette comprises a guide RNA (gRNA) or guide DNA (gDNA), the gDNA or gRNA targets a region in the 5′ GSH-HA and/or in the 3′ GSH-HA.

In some embodiments, a ceDNA vector described herein for integration of a nucleic acid of interest into a GSH locus can comprise: a first ITR, a guide RNA (gRNA) or guide DNA (gDNA) which targets a region in the GSH locus, a nucleic acid of interest and/or an expressible transgene cassette (e.g., a sequence that encodes a therapeutic protein or nucleic acid as described herein, and/or a reporter protein), and a second ITR. In some embodiments, such a ceDNA vector can comprise a first ITR only (e.g., a 5′ ITR but does not comprise a 3′ ITR). In alternative embodiments, such ceDNA vectors can comprise a second ITR only (e.g., it has a 3′ ITR and does not comprise a 5′ ITR).

Accordingly, some aspects of the technology described herein relate to a ceDNA vector useful for insertion of a GOI or transgene into a GSH as identified using the methods disclosed herein, where the ceDNA vector comprises ITR sequences selected from any of: (i) at least one WT ITR and at least one modified AAV inverted terminal repeat (ITR) (e.g., asymmetric modified ITRs); (ii) two modified ITRs where the mod-ITR pair have a different three-dimensional spatial organization with respect to each other (e.g., asymmetric modified ITRs), or (iii) symmetrical or substantially symmetrical WT-WT ITR pair, where each WT-ITR has the same three-dimensional spatial organization, or (iv) symmetrical or substantially symmetrical modified ITR pair, where each mod-ITR has the same three-dimensional spatial organization. The ceDNA vectors disclosed herein can be produced in eukaryotic cells, thus devoid of prokaryotic DNA modifications and bacterial endotoxin contamination in insect cells.

In some embodiments, the methods and ceDNA vectors as described herein allow insertion of a GOI or transgene into a safe harbor in a subject. The control of the expression of the GOI or transgene from the safe harbor can be regulated using regulatory switches has disclosed herein. One advantage of the ceDNA vector and methods as described herein allows one to safely insert a transgene into the genome of a host cell thereby preventing or avoiding adverse side effects that can occur when insertion of a transgene or GOI occurs at a non-safe harbor genomic loci or site. Moreover, insertion of a GOI or transgene into a GSH using the ceDNA vectors as disclosed herein is useful to enable continued expression of the transgene or GOI using the hosts cell's cellular machinery and post-translational modifications, thereby having to avoid repeat administrations of the ceDNA vector, and/or controlling the expression of the GOI or transgene by way of using the regulatory switches, as disclosed herein, and/or optimally processing the expressed protein with the host cells' post-transcriptional modification machinery.

In some embodiments, the disclosure also relates to a nucleic acid vector composition which is a closed end DNA (ceDNA) vector, comprising at least a portion or region of the GSH identified using the methods disclosed herein. In some embodiments, the portion or region of the GSH present in a ceDNA vector can be modified, e.g., insertion of a transgene or alternatively, introduction of a point mutation (e.g., insertion, deletion, any disruption of the gene), or a stop codon to disrupt or knock-out the gene function of a GSH gene identified herein, which is useful for example, to validate and/or characterize the identified GSH loci. In other embodiments, the portion or region of the GSH in the ceDNA vector can be modified to comprise a guide RNA (gRNA) inserted, e.g., a guide RNA for a nuclease as disclosed herein. In some embodiments, the ceDNA GSH vector can comprise a target site for a guide RNA (gRNA) as disclosed herein, or alternatively, a restriction cloning site for introduction of a nucleic acid of interest as disclosed herein.

In alternative embodiments, the disclosure herein also relates to a closed end DNA (ceDNA) nucleic acid vector composition comprising at GSH 5′-homology arm, and a GSH 3′-homology arm flanking a nucleic acid comprising a restriction cloning site, where the ceDNA vector can be used to integrate the flanked nucleic acid into the genome at a GSH by homologous recombination.

Aspects of the invention relate to methods to produce a ceDNA vector useful for insertion of a GOI or transgene into a GSH as identified using the methods disclosed herein. In all aspects, the capsid free, non-viral DNA vector (ceDNA vector) for insertion of a GOI or transgene into a GSH is obtained from a plasmid (referred to herein as a “ceDNA-plasmid”) comprising a polynucleotide expression construct template comprising in this order: a first 5′ inverted terminal repeat (e.g. AAV ITR); a heterologous nucleic acid sequence; and a 3′ ITR (e.g. AAV ITR), where the 5′ ITR and 3′ITR can be asymmetric relative to each other, or symmetric (e.g., WT-ITRs or modified symmetric ITRs) as defined herein.

A ceDNA vector for insertion of a GOI or transgene into a GSH as described herein is obtainable by a number of means that would be known to the ordinarily skilled artisan after reading this disclosure. For example, a polynucleotide expression construct template used for generating the ceDNA vectors of the present invention can be a ceDNA-plasmid (e.g. see FIG. 4B), a ceDNA-bacmid, and/or a ceDNA-baculovirus. In one embodiment, the ceDNA-plasmid comprises a restriction cloning site (e.g. SEQ ID NO: 123 and/or 124 operably positioned between the ITRs where a HA-L and HA-R can be inserted, and where an expression cassette comprising e.g., a promoter operatively linked to a GOI or transgene, e.g., a reporter gene and/or a therapeutic gene) can be inserted. In some embodiments, ceDNA vectors are produced from a polynucleotide template (e.g., ceDNA-plasmid, ceDNA-bacmid, ceDNA-baculovirus) containing symmetric or asymmetric ITRs (modified or WT ITRs).

In a permissive host cell, in the presence of e.g., Rep, the polynucleotide template having at least two ITRs replicates to produce ceDNA vectors. ceDNA vector production undergoes two steps: first, excision (“rescue”) of template from the template backbone (e.g. ceDNA-plasmid, ceDNA-bacmid, ceDNA-baculovirus genome etc.) via Rep proteins, and second, Rep mediated replication of the excised ceDNA vector. Rep proteins and Rep binding sites of the various AAV serotypes are well known to those of ordinary skill in the art. One of ordinary skill understands to choose a Rep protein from a serotype that binds to and replicates the nucleic acid sequence based upon at least one functional ITR. For example, if the replication competent ITR is from AAV serotype 2, the corresponding Rep would be from an AAV serotype that works with that serotype such as AAV2 ITR with AAV2 or AAV4 Rep but not AAV5 Rep, which does not. Upon replication, the covalently-closed ended ceDNA vector continues to accumulate in permissive cells and ceDNA vector is preferably sufficiently stable over time in the presence of Rep protein under standard replication conditions, e.g. to accumulate in an amount that is at least 1 pg/cell, preferably at least 2 pg/cell, preferably at least 3 pg/cell, more preferably at least 4 pg/cell, even more preferably at least 5 pg/cell.

Accordingly, one aspect of the invention relates to a process of producing a ceDNA vector for insertion of a GOI or transgene into a GSH as described herein, comprising the steps of: a) incubating a population of host cells (e.g. insect cells) harboring the polynucleotide expression construct template (e.g., a ceDNA-plasmid, a ceDNA-bacmid, and/or a ceDNA-baculovirus), which is devoid of viral capsid coding sequences, in the presence of a Rep protein under conditions effective and for a time sufficient to induce production of the ceDNA vector within the host cells, and wherein the host cells do not comprise viral capsid coding sequences; and b) harvesting and isolating the ceDNA vector from the host cells. The presence of Rep protein induces replication of the vector polynucleotide with a modified ITR to produce the ceDNA vector in a host cell. However, no viral particles (e.g. AAV virions) are expressed. Thus, there is no virion-enforced size limitation.

The presence of the ceDNA vector for insertion of a GOI or transgene into a GSH as described herein is isolated from the host cells can be confirmed by digesting DNA isolated from the host cell with a restriction enzyme having a single recognition site on the ceDNA vector and analyzing the digested DNA material on denaturing and non-denaturing gels to confirm the presence of characteristic bands of linear and continuous DNA as compared to linear and non-continuous DNA.

In another embodiment of this aspect and all other aspects provided herein, the GOI or transgene in a ceDNA vector for insertion of a GOI or transgene into a GSH as described herein is therapeutic transgene, e.g., a protein of interest, including but not limited to, a receptor, a toxin, a hormone, an enzyme, or a cell surface protein, an antibody or fusion protein. In another embodiment of this aspect and all other aspects provided herein, the protein of interest is a receptor. In another embodiment of this aspect and all other aspects provided herein, the protein of interest is an enzyme. Exemplary genes to be targeted and proteins of interest are described in detail in the methods of use and methods of treatment sections herein. In some embodiments, the transgene or GOI is selected from any of: a nucleic acid, an inhibitor, peptide or polypeptide, antibody or antibody fragment, fusion protein, antigen, antagonist, agonist, RNAi molecule, etc. In some embodiments, transgene or GOI encodes an inhibitor protein, for example, but not limited to, an antibody or antigen-binding fragment, or a fusion protein. In some embodiments, the transgene or GOI replaces a defective protein or a protein that is not being expressed or being expressed at low levels in the subject.

In some embodiments, the GOI or transgene when present in the ceDNA vector, or inserted into the GSH of a host's cells genome, it is under the control of a regulatory switch, as defined herein. In some embodiments, a ceDNA vector as disclosed herein, comprises two ITRs flanking a HA-L and a HA-R, wherein located between the HA-L and the HA-R is at least one heterologous nucleotide sequence (e.g., GOI or transgene) under the control of at least one regulatory switch, for example, at least one regulatory switch is selected from a binary regulatory switch, a small molecule regulatory switch, a passcode regulatory switch, a nucleic acid-based regulatory switch, a post-transcriptional regulatory switch, a radiation-controlled or ultrasound controlled regulatory switch, a hypoxia-mediated regulatory switch, an inflammatory response regulatory switch, a shear-activated regulatory switch, and a kill switch. Regulatory switches are disclosed herein in more detail below. In all aspects herein, the transgene or GOI encodes a therapeutic protein and when inserted into a GSH as disclosed herein, can be expressed at a desired level of expression, which can be a therapeutically effective amount of the therapeutic protein or genetic medicine.

In some embodiments, a ceDNA vector for insertion of a GOI or transgene into a GSH as described herein comprises two inverted terminal repeat sequences (ITRs) that are AAV ITRs, and can be, e.g., AAV-2, or any ITR selected from Table 5, or AAV1, AAV3, AAV4, AAV5, AAV 5, AAV7, AAV8, AAV9, AAV10, AAV 11, AAV12, AAVrh8, AAVrh10, AAV-DJ, and AAV-DJ8. In some embodiments, at least one ITR comprises a functional terminal resolution site and a Rep binding site. In some embodiments, the flanking ITRs in a ceDNA vector for insertion of a GOI or transgene into a GSH as described herein are symmetric or substantially symmetrical or asymmetric, as defined herein. In some embodiments, one or both of the ITRs are wild type, or wherein both of the ITRs are wild-type. In some embodiments, the flanking ITRs are from different viral serotypes. In some embodiments, where the flanking ITRs are both wild type, they can be selected from any AAV serotype as shown in Table 5. In some embodiments, the flanking ITRs in a ceDNA vector for insertion of a GOI or transgene into a GSH as described herein can comprise a sequence selected from the sequences in Tables 6, 8A, 8B or 9 herein.

In some embodiments, at least one of the ITRs in a ceDNA vector for insertion of a GOI or transgene into a GSH as described herein is altered from a wild-type AAV ITR sequence by a deletion, addition, or substitution that affects the overall three-dimensional conformation of the ITR. In some embodiments, one or both of the ITRs in a ceDNA vector for insertion of a GOI or transgene into a GSH as described herein is derived from an AAV serotype selected from AAV1, AAV2, AAV3, AAV4, AAV5, AAV6, AAV7, AAV8, AAV9, AAV10, AAV11, and AAV12.

In some embodiments, one or both of the ITRs in a ceDNA vector for insertion of a GOI or transgene into a GSH as described herein are synthetic. In some embodiments, one or both of the ITRs is not a wild type ITR, or wherein both of the ITRs are not wild-type.

In some embodiments, one or both of the ITRs in a ceDNA vector for insertion of a GOI or transgene into a GSH as described herein is modified by a deletion, insertion, and/or substitution in at least one of the ITR regions selected from A, A′, B, B′, C, C′, D, and D′. In some embodiments, a deletion, insertion, and/or substitution results in the deletion of all or part of a stem-loop structure normally formed by the A, A′, B, B′ C, or C′ regions. In some embodiments, one or both of the ITRs are modified by a deletion, insertion, and/or substitution that results in the deletion of all or part of a stem-loop structure normally formed by the B and B′ regions. In some embodiments, one or both of the ITRs are modified by a deletion, insertion, and/or substitution that results in the deletion of all or part of a stem-loop structure normally formed by the C and C′ regions. In some embodiments, one or both of the ITRs are modified by a deletion, insertion, and/or substitution that results in the deletion of part of a stem-loop structure normally formed by the B and B′ regions and/or part of a stem-loop structure normally formed by the C and C′ regions. In some embodiments, one or both of the ITRs comprise a single stem-loop structure in the region that normally comprises a first stem-loop structure formed by the B and B′ regions and a second stem-loop structure formed by the C and C′ regions. In some embodiments, one or both of the ITRs comprise a single stem and two loops in the region that normally comprises a first stem-loop structure formed by the B and B′ regions and a second stem-loop structure formed by the C and C′ regions.

In some embodiments, both ITRs in a ceDNA vector for insertion of a GOI or transgene into a GSH as described herein are altered in a manner that results in an overall three-dimensional symmetry when the ITRs are inverted relative to each other.

Other aspects of the invention relate to methods to integrate a nucleic acid of interest into a genome at a GSH identified herein using the methods and ceDNA vector compositions useful for insertion of a GOI or transgene into a GSH as disclosed herein. Other aspects relate to a cell, or transgenic animal with a nucleic acid of interest integrated into the genome using the methods and ceDNA vector compositions as disclosed herein.

In certain embodiments, a ceDNA vector for insertion of a GOI or transgene at a GSH as described herein can be monitored with appropriate biomarkers from treated patients to assess the efficiency of the gene insertion. In another aspect, there is provided a method of generating a genetically modified animal by using the gene knock-in system described herein using a ceDNA vector for insertion of a transgene at a GSH loci as described herein in accordance with the present disclosure.

In certain embodiments, the present disclosure relates to methods of using a ceDNA vector for insertion of a transgene at a GSH loci as described herein for inserting a donor sequence at a predetermined GSH insertion site or loci on a chromosome of a host cell, such as a eukaryotic or prokaryotic cell.

In some embodiments, the present application may be defined in any of the following paragraphs:
1. A capsid free, linear, closed-ended DNA (ceDNA) vector comprising at least one inverted terminal repeat (ITR) or two inverted terminal repeats (ITRs), at least one heterologous nucleotide sequence, and at least one Genomic Safe Harbor Homology Arm (GSH HA), wherein the GSH HA binds to a target site located in a genomic safe harbor locus (GSH locus) in Table 1A or Table 1B, and wherein the GSH HA guides insertion of the heterologous nucleotide sequence into a locus located within the genomic safe harbor, and in some embodiments, where there are two ITRs, the heterologous nucleotide sequence is located between the two ITRs.
2. The ceDNA vector of paragraph 1, wherein the ceDNA comprises at least a 5′ Genomic Safe Harbor Homology Arm (5′ GSH HA) or a 3′ Genomic Safe Harbor Homology Arm (3′ GSH HA), or both, wherein the 5′ GSH HA and the 3′ GSH HA bind to a target site located in a genomic safe harbor locus (GSH locus) in Table 1A or Table 1B, and wherein the 5′ GSH HA and/or the 3′ GSH HA guide insertion of the heterologous nucleotide sequence into a locus located within the genomic safe harbor.
3. The ceDNA vector of paragraph 2, wherein the heterologous nucleotide sequence is 3′ of the 5′ GSH HA, or 5′ of the 3′ GSH HA.
4. The ceDNA vector of paragraph 2, wherein the heterologous nucleotide sequence is located between the 5′ GSH HA and the 3′ GSH HA.
5. The ceDNA vector of paragraph 1, wherein insertion is by homologous recombination, homology direct repair (HDR), or non-homologous end joining (NHEJ).
6. The ceDNA vector of paragraph 1, wherein the at least a portion of the GSH locus comprises the PAX5 genomic DNA or a fragment thereof.
7. The ceDNA vector of paragraph 1, wherein the GSH locus is an untranslated sequence or an intron or exon of the PAX5 gene, or an untranslated sequence or an intron or exon of the KIF6 gene.
8. The ceDNA vector of paragraph 1, wherein the target site is in the PAX5 GSH locus or KIF6, and is a region of at least 100-1000 nucleotides located in Chromosome 9 (36,833,275-37,034,185 reverse strand) or Chromosome 6 (39,329,990-39,725,405).
9. The ceDNA vector of paragraph 1, wherein the GSH locus is a nucleic acid selected from any of the nucleic acid sequences listed in Table 1A or 1B.
10. The ceDNA vector of paragraph 1, wherein the GSH locus is a region in any of the untranslated sequence or an intron or exon of the genes selected from Kif6, KLHL7, NUPL2, mir684, KCNH2, GPNMB, MIR4540, MIR4475, MIR4476, PRL32P21, LOC105376031, LOC105376032, LOC105376030, MELK, EBLN3P, ZCCHC7, RNF38
11. The ceDNA vector of paragraph 1, wherein the GSH locus is a region in any of the untranslated sequence or an intron or exon within any of the chromosomal regions selected from: chromosome 9 (36,833,275-37,034,185) (Pax6); Chromosome 6 (39,329,990-39,725,405) (Kif6) or Chromosome 16 (cdh 8: 61,647,242-62,036,835 cdh 11: 64,943,753-65,122,198).
12. The ceDNA vector of paragraph 1, wherein the GSH locus is a region in any of the untranslated sequence or an intron or exon of the genes selected from Accession numbers: NC_000009.12 (36833274 . . . 37035949, complement); NC_000009.12 (36864254 . . . 36864308, complement); NC_000009.12 (36823539 . . . 36823599, complement); NC_000009.12 (36893462 . . . 36893531, complement), NC_000009.12 (37046835 . . . 37047242); NC_000009.12 (37027763 . . . 37031333); NC_000009.12 (37002697 . . . 37007774); NC_000009.12 (36779475 . . . 36830456); NC_000009.12 (36572862 . . . 36677683); NC_000009.12 (37079896 . . . 37090401); NC_000009.12 (37120169 . . . 37358149) or NC_000009.12 (36336398 . . . 36487384, complement).
13. A capsid free, linear, closed-ended DNA (ceDNA) vector comprising at least one ITR, or alternatively, two inverted terminal repeats (ITRs), and located between the two ITRs, a gene editing cassette, at least one heterologous nucleotide sequence, and at least one Genomic Safe Harbor Homology Arm (GSH HA), wherein the gene editing cassette comprises at least one gene editing molecule selected from a nuclease, a guide RNA (gRNA), a guide DNA (gDNA), and an activator RNA, and wherein the GSH HA binds to a target site located in a genomic safe harbor locus (GSH locus) in Table 1A or Table 1B, and wherein the GSH HA guides insertion of the heterologous nucleotide sequence into a locus located within the genomic safe harbor.
14. A capsid free, linear, closed-ended DNA (ceDNA) vector comprising at least one ITR, or alternatively two inverted terminal repeats (ITRs), and located between the two ITRs, at least one a guide RNA (gRNA) or at least one guide DNA (gDNA), and at least one heterologous nucleotide sequence, wherein the at least one gRNA or at least one gDNA binds to a target site located in a genomic safe harbor locus (GSH locus) in Table 1A or Table 1B, and wherein the gDNA or gRNA guides insertion of the heterologous nucleotide sequence into a locus located within the genomic safe harbor.
15. The ceDNA vector of paragraph 13 or 14, wherein the target site is in the PAX5 GSH locus or KIF6 GSH locus, and is a region of at least 100-1000 nucleotides located in Chromosome 9 (36,833,275-37,034,185 reverse strand), or Chromosome 6 (39,329,990-39,725,405).
16. The ceDNA vector of paragraph 13 or 14, wherein the GSH locus is a nucleic acid selected from any of the nucleic acid sequences listed in Table 1A or 1B.
17. The ceDNA vector of paragraph 13 or 14, wherein the GSH locus is a region in any of the untranslated sequence or an intron or exon of the genes selected from Kif6, KLHL7, NUPL2, mir684, KCNH2, GPNMB, MIR4540, MIR4475, MIR4476, PRL32P21, LOC105376031, LOC105376032, LOC105376030, MELK, EBLN3P, ZCCHC7, RNF38
18. The ceDNA vector of paragraph 13 or 14, wherein the GSH locus is a region in any of the untranslated sequence or an intron or exon within any of the chromosomal regions selected from: chromosome 9 (36,833,275-37,034,185) (Pax6); Chromosome 6 (39,329,990-39,725,405) (Kif6) or Chromosome 16 (cdh 8: 61,647,242-62,036,835 cdh 11: 64,943,753-65,122,198).
19. The ceDNA vector of paragraph 13 or 14, wherein the GSH locus is a region in any of the untranslated sequence or an intron or exon of the genes selected from Accession numbers: NC_000009.12 (36833274 . . . 37035949, complement); NC_000009.12 (36864254 . . . 36864308, complement); NC_000009.12 (36823539 . . . 36823599, complement); NC_000009.12 (36893462 . . . 36893531, complement), NC_000009.12 (37046835 . . . 37047242); NC_000009.12 (37027763 . . . 37031333); NC_000009.12 (37002697 . . . 37007774); NC_000009.12 (36779475 . . . 36830456); NC_000009.12 (36572862 . . . 36677683); NC_000009.12 (37079896 . . . 37090401); NC_000009.12 (37120169 . . . 37358149) or NC_000009.12 (36336398 . . . 36487384, complement).
20. The ceDNA vector of paragraph 13, wherein the ceDNA comprises at least a 5′ Genomic Safe Harbor Homology Arm (5′ GSH HA) or a 3′ Genomic Safe Harbor Homology Arm (3′ GSH HA), or both, wherein the 5′ GSH HA and the 3′ GSH HA bind to a target site located in a genomic safe harbor locus (GSH locus) in Table 1A or Table 1B, and wherein the 5′ GSH HA and/or the 3′ GSH HA guide insertion of the heterologous nucleotide sequence into a locus located within the genomic safe harbor.
21. The ceDNA vector of paragraph 20, wherein the heterologous nucleotide sequence is 3′ of the 5′ GSH HA, or 5′ of the 3′ GSH HA.
22. The ceDNA vector of paragraph 20, wherein the heterologous nucleotide sequence is located between the 5′ GSH HA and the 3′ GSH HA.
23. The ceDNA vector of paragraph 13 or 14, wherein insertion is by homologous recombination, homology direct repair (HDR), or non-homologous end joining (NHEJ).
24. The ceDNA vector of paragraph 13, wherein at least one gene editing molecule is a nuclease.
25. The ceDNA vector of paragraph 24, wherein the nuclease is a sequence specific nuclease or a nucleic acid-guided nuclease.
26. The ceDNA vector of paragraph 25, wherein the sequence specific nuclease is selected from a nucleic acid-guided nuclease, zinc finger nuclease (ZFN), a meganuclease, a transcription activator-like effector nuclease (TALEN), or a megaTAL.
27. The ceDNA vector of paragraph 26, wherein the sequence specific nuclease is a nucleic acid-guided nuclease selected from a single-base editor, an RNA-guided nuclease, and a DNA-guided nuclease.
28. The ceDNA vector of paragraph 13, wherein at least one gene editing molecule is a guide RNA (gRNA) or a guide DNA (gDNA), wherein the gRNA or gDNA binds to a region in the at least one GSH homology arm, or binds to a target site located in a genomic safe harbor locus (GSH locus) in Table 1A or Table 1B.
29. The ceDNA vector of paragraph 28, wherein the target site is in the PAX5 GSH locus, and is a region of at least 100-1000 nucleotides located in Chromosome 9 (36,833,275-37,034,185 reverse strand).
30. The ceDNA vector of paragraph 13, wherein at least one gene editing molecule is an activator RNA.
31. The ceDNA of any one of paragraphs 25, wherein the nucleic acid-guided nuclease is a CRISPR nuclease.
32. The ceDNA vector of paragraph 31, wherein the CRISPR nuclease is a Cas nuclease.
33. The ceDNA vector of paragraph 32, wherein the Cas nuclease is selected from Cas9, nicking Cas9 (nCas9), and deactivated Cas (dCas).
34. The ceDNA vector of paragraph 33, wherein the nCas9 contains a mutation in the HNH or RuVc domain of Cas.
35. The ceDNA vector of paragraph 33, wherein the dCas is fused to a heterologous transcriptional activation domain that can be directed to a promoter region.
36. The ceDNA vector of any one of paragraphs 33-36, wherein the dCas is S. pyogenes dCas9.
37. The ceDNA vector of any one of paragraphs 14 or 28-36, wherein the guide RNA (gRNA) or guide DNA (gDNA) sequence binds to a region in the at least one GSH homology arm, or binds to a target site located in a genomic safe harbor locus (GSH locus) in Table 1A or Table 1B and CRISPR silences the target gene (CRISPRi system).
38. The ceDNA vector of any one of paragraphs 14 or 28 or 37, wherein the guide RNA (gRNA) or guide DNA (gDNA) sequence targets a target site located in the 5′ GSH homology arm and activates insertion of the heterologous nucleic acid (CRISPRa system).
39. The ceDNA vector of any one of paragraphs 13, 14 or 28, wherein the at least one gene editing molecule comprises a first guide RNA and a second guide RNA.
40. The ceDNA vector of paragraph 13, 14 or 28 or 39, wherein gDNA or gRNA effects non-homologous end joining (NHEJ) and insertion of the heterologous nucleic acid into a GSH locus.
41. The ceDNA vector of any one of paragraphs 14 or 39, wherein the vector encodes multiple copies of one guide RNA sequence.
42. The ceDNA vector of paragraph 24, wherein a gene editing cassette comprises a first regulatory sequence operably linked to a nucleotide sequence that encodes a nuclease.
43. The ceDNA vector of paragraph 42, wherein the first regulatory sequence comprises a promoter.
44. The ceDNA vector of paragraph 43, wherein the promoter is CAG, Pol III, U6, or H1.
45. The ceDNA vector of any one of paragraphs 42-44, wherein the first regulatory sequence comprises a modulator.
46. The ceDNA vector of paragraph 45, wherein the modulator is selected from an enhancer and a repressor.
47. The ceDNA vector of any one of paragraphs 42-47, wherein the first heterologous nucleotide sequence comprises an intron sequence upstream of the nucleotide sequence that encodes the nuclease, wherein the intron sequence comprises a nuclease cleavage site.
48. The ceDNA vector of paragraph 42, wherein the gene editing cassette comprises a second heterologous nucleotide sequence comprises a second regulatory sequence operably linked to a nucleotide sequence that encodes a guide RNA (gRNA) or guide DNA (gDNA).
49. The ceDNA vector of paragraph 48, wherein the second regulatory sequence comprises a promoter.
50. The ceDNA vector of paragraph 49, wherein the promoter is CAG, Pol III, U6, or H1.
51. The ceDNA vector of any one of paragraphs 48-50, wherein the second regulatory sequence comprises a modulator.
52. The ceDNA vector of paragraph 51, wherein the modulator is selected from an enhancer and a repressor.
53. The ceDNA vector of paragraph 48, wherein the gene editing cassette comprises a third heterologous nucleotide sequence comprising a third regulatory sequence operably linked to a nucleotide sequence that encodes an activator RNA.
54. The ceDNA vector of paragraph 53, wherein the third regulatory sequence comprises a promoter.
55. The ceDNA vector of paragraph 54, wherein the promoter is CAG, Pol III, U6, or H1.
56. The ceDNA vector of any one of paragraphs 53-55, wherein the third regulatory sequence comprises a modulator.
57. The ceDNA vector of paragraph 56, wherein the modulator is selected from an enhancer and a repressor.
58. The ceDNA vector of any of paragraphs 1-57, wherein the target site in the GSH locus is at least 1 kb in length.
59. The ceDNA vector of any of paragraphs 1-57, wherein the target site in the GSH locus is between 300-3 kb in length.
60. The ceDNA vector of any of paragraphs 1-57, wherein the target site in the GSH locus comprises a target site for a guide RNA (gRNA) or guide RNA (gRNA).
61. The ceDNA vector of any of paragraphs 13, 14, 37, 48 and 60, wherein the gRNA or gDNA is for a sequence-specific nuclease selected from any of: a TAL-nuclease, a zinc-finger nuclease (ZFN), a meganuclease, a megaTAL, or an RNA guide endonuclease (e.g., CAS9, cpf1, nCAS9).
62. The ceDNA vector of any of paragraphs 1-61, wherein at least one ITR comprises a functional terminal resolution site and a Rep binding site.
63. The ceDNA vector of any of paragraphs 1-62, wherein the two ITRs are AAV ITRs.
64. The ceDNA vector of paragraph 63, wherein the AAV ITRs are AAV2 ITRs.
65. The ceDNA vector of any of paragraphs 1-64, wherein the flanking ITRs are symmetric or asymmetric.
66. The ceDNA vector of any of paragraphs 1-65, wherein the flanking ITRs are symmetrical or substantially symmetrical.
67. The ceDNA vector of any of paragraphs 1-66, wherein the flanking ITRs are asymmetric.
68. The ceDNA vector of any of paragraphs 1-67, wherein one or both of the ITRs are wild type, or wherein both of the ITRs are wild-type.
69. The ceDNA vector of any of paragraphs 1-68, wherein the flanking ITRs are from different viral serotypes.
70. The ceDNA vector of any of paragraphs 1-69, wherein one or both of the ITRs comprises a sequence selected from the sequences in Tables 6, 8A, 8B or 9.
71. The ceDNA vector of any of paragraphs 1-70, wherein at least one of the ITRs is altered from a wild-type AAV ITR sequence by a deletion, addition, or substitution that affects the overall three-dimensional conformation of the ITR.
72. The ceDNA vector of any of paragraphs 1-71, wherein one or both of the ITRs are derived from an AAV serotype selected from AAV1, AAV2, AAV3, AAV4, AAV5, AAV6, AAV7, AAV8, AAV9, AAV10, AAV11, and AAV12.
73. The ceDNA vector of any of paragraphs 1-72, wherein one or both of the ITRs are synthetic.
74. The ceDNA vector of any of paragraphs 1-73, wherein one or both of the ITRs is not a wild type ITR, or wherein both of the ITRs are not wild-type.
75. The ceDNA vector of any of paragraphs 1-74, wherein one or both of the ITRs is modified by a deletion, insertion, and/or substitution in at least one of the ITR regions selected from A, A′, B, B′, C, C′, D, and D′.
76. The ceDNA vector of any of paragraphs 1-75, wherein the deletion, insertion, and/or substitution results in the deletion of all or part of a stem-loop structure normally formed by the A, A′, B, B′ C, or C′ regions.
77. The ceDNA vector of any of paragraphs 1-76, wherein one or both of the ITRs are modified by a deletion, insertion, and/or substitution that results in the deletion of all or part of a stem-loop structure normally formed by the B and B′ regions.
78. The ceDNA vector of any of paragraphs 1-77, wherein one or both of the ITRs are modified by a deletion, insertion, and/or substitution that results in the deletion of all or part of a stem-loop structure normally formed by the C and C′ regions.
79. The ceDNA vector of any of paragraphs 1-78, wherein one or both of the ITRs are modified by a deletion, insertion, and/or substitution that results in the deletion of part of a stem-loop structure normally formed by the B and B′ regions and/or part of a stem-loop structure normally formed by the C and C′ regions.
80. The ceDNA vector of any of paragraphs 1-79, wherein one or both of the ITRs comprise a single stem-loop structure in the region that normally comprises a first stem-loop structure formed by the B and B′ regions and a second stem-loop structure formed by the C and C′ regions.
81. The ceDNA vector of any of paragraphs 1-80, wherein one or both of the ITRs comprise a single stem and two loops in the region that normally comprises a first stem-loop structure formed by the B and B′ regions and a second stem-loop structure formed by the C and C′ regions.
82. The ceDNA vector of any of paragraphs 1-82, wherein both ITRs are altered in a manner that results in an overall three-dimensional symmetry when the ITRs are inverted relative to each other.
83. The ceDNA vector of any of paragraphs 1-82, wherein at least one heterologous nucleotide sequence is under the control of at least one regulatory switch or promoter.
84. The ceDNA vector of paragraph 83, wherein at least one regulatory switch is selected from a binary regulatory switch, a small molecule regulatory switch, a passcode regulatory switch, a nucleic acid-based regulatory switch, a post-transcriptional regulatory switch, a radiation-controlled or ultrasound controlled regulatory switch, a hypoxia-mediated regulatory switch, an inflammatory response regulatory switch, a shear-activated regulatory switch, and a kill switch.
85. The ceDNA vector of paragraph 84, wherein the promoter is an inducible promoter, or a tissue specific promoter or a constitutive promoter.
86. The ceDNA vector of any of paragraphs 1-13 or 20-22, wherein the 5′ or 3′ GSH homology arms, or both are between 30-2000 bp in length.
87. The ceDNA vector of any of paragraphs 1-86, wherein the heterologous nucleic acid comprises a transgene, and wherein the transgene is selected from any of: a nucleic acid, an inhibitor, peptide or polypeptide, antibody or antibody fragment, fusion protein, antigen, antagonist, agonist, RNAi molecule, miRNA, etc.
88. The ceDNA vector of any of paragraphs 1-87, wherein heterologous nucleic acid sequence is in an orientation for integration into the genome at the GSH locus in a forward orientation.
89. The ceDNA vector of any of paragraphs 1-88, wherein n heterologous nucleic acid sequence is in an orientation for integration into the genome at the GSH locus in a reverse orientation.
90. The ceDNA vector of any of paragraphs 4, 13 or 20-22, wherein 5′ GSH homology arm and the 3′ GSH homology arm bind to target sites that are spatially distinct nucleic acid sequences in the genomic safe harbor locus disclosed in Tables 1A or 1B.
91. The ceDNA vector of any of paragraphs 1-4, 13 or 20-22, wherein the at least one GSH-HA or GSH 5′ homology arm, or GSH 3′ homology arm are at least 65% complementary to a target sequence in the genomic safe harbor locus in Table 1A or Table 1B.
92. The ceDNA vector of any of paragraphs 1-4, 13 or 20-22, wherein the at least one GSH-HA or 5′ GSH homology arm, or the GSH 3′ homology arm bind to a target site located in the PAX5 genomic safe harbor locus sequence.
93. The ceDNA vector of any of paragraphs 1-4, 13 or 20-22, wherein the at least one GSH-HA, or 5′ GSH homology arm, or the GSH 3′ homology arm are at least 65% complementary to at least part the PAX5 genomic safe harbor locus sequence.
94. The ceDNA vector of any of paragraphs 1-4, 13 or 20-22, wherein the at least GSH-HA, or 5′ GSH homology arm or the 3′ GSH homology arm bind to a target site located in a GSH locus located in a gene selected from Table 1A or 1B.
95. The ceDNA vector of any one of paragraphs 1-94, comprising a first endonuclease restriction site upstream of the 5′ homology arm and/or a second endonuclease restriction site downstream of the 3′ homology arm.
96. The ceDNA vector of paragraph 95, wherein the first endonuclease restriction site and the second endonuclease restriction site are the same restriction endonuclease sites.
97. The ceDNA vector of paragraph 95-96, wherein at least one endonuclease restriction site is cleaved by a nuclease or endonuclease which is also encoded by a nucleic acid present in the gene editing cassette.
98. The ceDNA vector of any one of paragraphs 1-97, wherein the heterologous nucleic acid or the gene editing cassette, or both, further comprises one or more poly-A sites.
99. The ceDNA vector of any one of paragraphs 1-98, wherein the ceDNA vector comprises at least one of a regulatory element and a poly-A site 3′ of the 5′ GSH homology arm and/or 5′ of the 3′ GSH homology arm.
100. The ceDNA vector of any one of paragraphs 1-99, where the heterologous nucleic acid further comprises a 2A and/or a nucleic acid encoding reporter protein 5′ of the 3′ GSH homology arm.
101. The ceDNA vector of any one of paragraphs 13, 24 or 48-57, wherein the gene editing cassette further comprises a nucleic acid sequence encoding an enhancer of homologous recombination.
102. The ceDNA vector of paragraph 102, wherein the enhancer of homologous recombination is selected from SV40 late polyA signal upstream enhancer sequence, the cytomegalovirus early enhancer element, an RSV enhancer, and a CMV enhancer.

103. The ceDNA vector of any of paragraphs 1-102, wherein the ceDNA vector is administered to a subject with a disease or disorder selected from cancer, autoimmune disease, a neurodegenerative disorder, hypercholesterolemia, acute organ rejection, multiple sclerosis, post-menopausal osteoporosis, skin conditions, asthma, or hemophilia.
104. The ceDNA vector of paragraph 103, wherein the cancer is selected from a solid tumor, soft tissue sarcoma, lymphoma, and leukemia.
105. The ceDNA vector of paragraph 103, wherein the autoimmune disease is selected from rheumatoid arthritis and Crohn's disease.
106. The ceDNA vector of paragraph 103, wherein the skin condition is selected from psoriasis and atopic dermatitis.
107. The ceDNA vector of paragraph 103, wherein the neurodegenerative disorder is Alzheimer's disease.
108. A cell comprising the ceDNA vector of any of paragraphs 1-102.
109. The cell of paragraph 108, wherein the cell is a red blood cell (RBC) or RBC precursor cell.
110. The cell of paragraph 108, wherein the RBC precursor cell is a CD44+ or CD34+ cell.
111. The cell of paragraph 108, wherein the cell is a stem cell.
112. The cell of paragraph 108, wherein the cell is an iPS cell or embryonic stem cell.
113. The cell of paragraph 108, wherein the iPS cell is a patient-derived iPSC.
114. The cell of any of paragraphs 108-113, wherein the cell is a mammalian cell.
115. The cell of paragraph 114, wherein the mammalian cell is a human cell.
116. The cell of paragraph 108, wherein the cell is ex vivo or in vivo, or in vitro.
117. The cell of paragraph 108, wherein the cell has been removed from a human subject.
118. The cell of paragraph 108, wherein the cell is present in a human or animal subject.
119. A kit comprising a ceDNA vector composition of any of paragraphs 1-102; and at least one of: (i) at least one GSH 5′ primer and at least one GSH 3′ primer, wherein the GSH locus is any shown in Table 1A or 1B, wherein the at least one GSH 5′ primer binds to a region of the GSH locus upstream of the site of integration, and the at least one GSH 3′ primer is at least binds to a region of the GSH downstream of the site of integration; and/or (ii) at least two GSH 5′ primers comprising a forward GSH 5′ primer that binds to a region of the GSH upstream of the site of integration, and a reverse GSH 5′ primer that binds to a sequence in the nucleic acid inserted at the site of integration in the GSH sequence, wherein the GSH locus is any shown in Table 1A or 1B; and/or (iii) at least two GSH 3′ primers comprising a forward GSH 3′ primer that binds to a sequence located at the 3′ end of the nucleic acid inserted at the site of integration in the GSH sequence, and a reverse GSH 3′ primer binds to a region of the GSH downstream of the site of integration, and wherein the GSH locus is any shown in Table 1A or 1B.
120. The kit of paragraph 119, wherein the ceDNA comprises at least one modified terminal repeat.
121. A kit comprising: (a) a GSH-specific single guide and an RNA guided nucleic acid sequence present in one or more ceDNA vectors; and (b) a ceDNA GSH knock-in vector comprising two inverted terminal repeats (ITRs), and located between the two ITRs, at least one heterologous nucleotide sequence located between a 5′ Genomic Safe Harbor Homology Arm (5′ GSH HA) and a 3′ Genomic Safe Harbor Homology Arm (3′ GSH HA), wherein the 5′ GSH HA and the 3′ GSH HA bind to a target site located in a genomic safe harbor locus (GSH locus) in Table 1A or Table 1B, and wherein the 5′ GSH HA and the 3′ GSH HA guide homologous recombination into a locus located within the genomic safe harbor, wherein one or more of the sequences of (a) or (b) are comprised on a ceDNA vector of any of paragraphs 1-120.
122. The kit of paragraph 121, wherein the ceDNA GSH knock-in vector is a GSH-CRISPR-Cas vector.
123. The kit of paragraph 121, wherein the GSH CRISPR-Cas vector comprises a GSH-sgRNA nucleic acid sequence and Cas9 nucleic acid sequence.
124. The kit of paragraph 121, wherein the 5′ GSH homology arm and the 3′ GSH homology arm are at least 65% complementary to a sequence in the genomic safe harbor (GSH) of Table 1A or 1B, and wherein the GSH 5′ and 3′ homology arms guide insertion by homologous recombination, of the nucleic acid sequence located between the GSH 5′ homology arm and a GSH 3′ homology arm into a GSH locus located within the genomic safe harbor of one in Table 1A or 1B.
125. The kit of paragraph 121, wherein the GSH knockin donor vector is a PAX5 knockin donor vector comprising a PAX5 5′ homology arm and a PAX5 3′ homology arm, wherein the PAX5 5′ homology arm and the PAX5 3′ homology arm are at least 65% complementary to the PAX5 genomic safe harbor locus, and wherein the PAX5 5′ and 3′ homology arms guide insertion, by homologous recombination, of the nucleic acid located between the GSH 5′ homology arm and a GSH 3′ homology arm into a locus within the PAX5 genomic safe harbor.
126. The kit of paragraph 121, wherein the GSH knockin donor vector is a knockin donor vector comprising a 5′ homology arm which binds to a GSH locus listed in Table 1A or 1B, and a 3′ homology arm which binds to a spatially distinct region of the same GSH locus that the 5′ homology arm binds to, wherein the 5′ and 3′ homology arms guide insertion, by homologous recombination, of the nucleic acid located between the GSH 5′ homology arm and a GSH 3′ homology arm into a GSH locus listed in Table 1A or 1B.
127. The kit of any of paragraphs 121, further comprising at least one GSH 5′ primer and at least one GSH 3′ primer, wherein the GSH is identified by the ceDNA vector of any of paragraphs 41 to 51, wherein the at least one GSH 5′ primer is at least 80% complementary to a region of the GSH upstream of the site of integration, and the at least one GSH 3′ primer is at least 80% complementary to a region of the GSH downstream of the site of integration.
128. The kit of any of paragraphs 121-127, further comprising at least two GSH 5′ primers comprising (a) a forward GSH 5′ primer that is at least 80% complementary to a region of the GSH upstream of the site of integration, and (b) a reverse GSH 5′ primer that is at least 80% complementary to a sequence in the nucleic acid inserted at the site of integration in the GSH sequence, wherein the GSH is identified by the ceDNA vector of any of paragraphs 41 to 51.
129. The kit of any of paragraphs 121-128, further comprising at least two GSH 3′ primers comprising; (a) a forward GSH 3′ primer that is at least 80% complementary to a sequence located at the 3′ end of the nucleic acid inserted at the site of integration in the GSH sequence, and (b) a reverse GSH 3′ primer that is at least 80% complementary to a region of the GSH downstream of the site of integration, and wherein the GSH is identified by the ceDNA vector of any of paragraphs 41 to 51.
130. The kit of any of paragraphs 121-129, wherein the GSH 5′ primer is a PAX5 5′ primer and the GSH 3′ primer is a PAX 3′ primer, wherein the PAX5 5′ primer and the PAX5 3′ primer flank the site of integration in the PAX5 genomic safe harbor.
131. A method of generating a genetically modified animal comprising a nucleic acid interest inserted at a PAX5 Genomic Safe Harbor (GSH) locus, comprising a) introducing into a host cell a ceDNA of any of paragraphs 1-102, and b) introducing the cell generated in (a) into a carrier animal to produce a genetically modified animal.
132. The ceDNA vector of paragraph 131, wherein the host cell is a zygote or a pluripotent stem cell.
133. A genetically modified animal produced by the ceDNA vector of paragraph 131. The methods and compositions described herein can be used in methods comprising homology recombination, for example, as described in Rouet et al. Proc Natl Acad Sci 91:6064-6068 (1994); Chu et al. Nat Biotechnol 33:543-548 (2015); Richardson et al. Nat Biotechnol 33:339-344 (2016); Komor et al. Nature 533:420-424 (2016); the contents of each of which are incorporated by reference herein in their entirety.

These and other aspects of the invention are described in further detail below.

### BRIEF DESCRIPTION OF THE DRAWINGS

Embodiments of the present disclosure, briefly summarized above and discussed in greater detail below, can be understood by reference to the illustrative embodiments of the disclosure depicted in the appended drawings. However, the appended drawings illustrate only typical embodiments of the disclosure and are therefore not to be considered limiting of scope, for the disclosure may admit to other equally effective embodiments.

FIG. 1A is a schematic of an exemplary ceDNA vector for insertion of a transgene (or GOI) into a genomic safe harbor loci (GSH loci) of the genome in a host cell. FIG. 1A shows a ceDNA vector which comprises two inverted terminal repeat (ITR) sequences flanking a left homology arm (also referred to as a HA-L or 5′ HA) and a right homology arm (HA-R), where the HA-L and HA-R flank a heterologous nucleic acid construct comprising at least one gene of interest (GOI) (or transgene) and an initiation start codon (arrow). In some embodiments, the GOI can be genomic DNA (gDNA) encoding a protein or nucleic acid of interest, where the GOI has an open reading frame (ORF) and comprises introns and exons. In some embodiments, the GOI can be complementary DNA (cDNA) (i.e., DNA lacking introns). In some embodiments, the GOI is operatively linked to any one or more of: a promoter or regulatory switch as defined herein, a 5′ UTR, a 3′ UTR, a polyadenylation sequence, post-transcriptional elements which is operatively linked to a promoter or other regulatory switch as described herein. The ITRs can be symmetric, asymmetric or substantially symmetric relative to each other, as defined herein. The exemplary ceDNA vector shown in FIG. 1A can be administered with one or more vectors, including a ceDNA vector expressing a gene editing molecule, such as those described in International Patent Application PCT/US18/64242, which is incorporated herein in its entirety by reference.

FIG. 1B illustrates an exemplary structure of a ceDNA vector for insertion of a GOI or transgene into a genomic safe harbor of a host cells' genome as disclosed herein, comprising asymmetric ITRs flanking the HA-L and HA-R. In this embodiment, the exemplary ceDNA vector comprises between the HA-L and HA-R regions, an expression cassette containing CAG promoter, WPRE, and BGHpA. An open reading frame (ORF) allows expression of a transgene inserted into the cloning site (R3/R4) between the CAG promoter and WPRE. The expression cassette is flanked by a HA-L and HA-R, which in turn are flanked by two inverted terminal repeats (ITRs)—the wild-type AAV2 ITR on the upstream (5′-end) and the modified ITR on the downstream (3′-end) of the expression cassette, therefore the two ITRs flanking the expression cassette are asymmetric with respect to each other.

FIG. 1C illustrates an exemplary structure of a ceDNA vector for insertion of a GOI or transgene into a genomic safe harbor of a host cells' genome as disclosed herein comprising asymmetric ITRs flanking the HA-L and HA-R, with an expression cassette containing CAG promoter, WPRE, and BGHpA. An open reading frame (ORF) allows expression of a transgene inserted into the cloning site between CAG promoter and WPRE. The expression cassette is flanked by a HA-L and HA-R, which in turn are flanked by two inverted terminal repeats (ITRs)—a modified ITR on the upstream (5′-end) and a wild-type ITR on the downstream (3′-end) of the expression cassette.

FIG. 1D illustrates an exemplary structure of a ceDNA vector for insertion of a GOI or transgene into a genomic safe harbor of a host cells' genome as disclosed herein comprising asymmetric ITRs flanking the HA-L and HA-R, with an expression cassette containing an enhancer/promoter, a transgene, a post transcriptional element (WPRE), and a polyA signal. An open reading frame (ORF) allows expression of a transgene into the cloning site between CAG promoter and WPRE. The expression cassette is flanked by a HA-L and HA-R, which in turn are flanked by two inverted terminal repeats (ITRs) that are asymmetrical with respect to each other; a modified ITR on the upstream (5′-end) and a modified ITR on the downstream (3′-end) of the expression cassette, where the 5′ ITR and the 3′ITR are both modified ITRs but have different modifications (i.e., they do not have the same modifications).

FIG. 1E illustrates an exemplary structure of a ceDNA vector for insertion of a GOI or transgene into a genomic safe harbor of a host cells' genome as disclosed herein, comprising symmetric modified ITRs, or substantially symmetrical modified ITRs as defined herein flanking the HA-L and HA-R, with an expression cassette containing CAG promoter, WPRE, and BGHpA. An open reading frame (ORF) allows expression of a transgene is inserted into the cloning site between CAG promoter and WPRE. The expression cassette is flanked by a HA-L and HA-R, which in turn are flanked by two modified inverted terminal repeats (ITRs), where the 5′ modified ITR and the 3′ modified ITR are symmetrical or substantially symmetrical.

FIG. 1F illustrates an exemplary structure of a ceDNA vector for insertion of a GOI or transgene into a genomic safe harbor of a host cells' genome as disclosed herein comprising symmetric modified ITRs, or substantially symmetrical modified ITRs as defined herein flanking the HA-L and HA-R, with an expression cassette containing an enhancer/promoter, a transgene, a post transcriptional element (WPRE), and a polyA signal. An open reading frame (ORF) allows expression of a transgene into the cloning site between CAG promoter and WPRE. The expression cassette is flanked by a HA-L and HA-R, which in turn are flanked by two modified inverted terminal repeats (ITRs), where the 5′ modified ITR and the 3′ modified ITR are symmetrical or substantially symmetrical.

FIG. 1G illustrates an exemplary structure of a ceDNA vector for insertion of a GOI or transgene into a genomic safe harbor of a host cells' genome as disclosed herein, comprising symmetric WT-ITRs, or substantially symmetrical WT-ITRs as defined herein flanking the HA-L and HA-R R, with an expression cassette containing CAG promoter, WPRE, and BGHpA. An open reading frame (ORF) allows expression of the transgene inserted into the cloning site between CAG promoter and WPRE. The expression cassette is flanked by a HA-L and HA-R, which in turn are flanked by two wild type inverted terminal repeats (WT-ITRs), where the 5′ WT-ITR and the 3′ WT ITR are symmetrical or substantially symmetrical.

FIG. 1H illustrates an exemplary structure of a ceDNA vector insertion of a GOI or transgene into a genomic safe harbor of a host cells' genome as disclosed herein, comprising symmetric modified ITRs, or substantially symmetrical modified ITRs as defined herein flanking the HA-L and HA-R, with an expression cassette containing an enhancer/promoter, a transgene, a post transcriptional element (WPRE), and a polyA signal. An open reading frame (ORF) allows expression of a transgene in the cloning site between CAG promoter and WPRE. The expression cassette is flanked by a HA-L and HA-R, which in turn are flanked by two wild type inverted terminal repeats (WT-ITRs), where the 5′ WT-ITR and the 3′ WT ITR are symmetrical or substantially symmetrical.

FIG. 2A provides the T-shaped stem-loop structure of a wild-type left ITR of AAV2 (SEQ ID NO: 52) with identification of A-A′ arm, B-B′ arm, C-C′ arm, two Rep binding sites (RBE and RBE′) and also shows the terminal resolution site (trs). The RBE contains a series of 4 duplex tetramers that are believed to interact with either Rep 78 or Rep 68. In addition, the RBE′ is also believed to interact with Rep complex assembled on the wild-type ITR or mutated ITR in the construct. The D and D′ regions contain transcription factor binding sites and other conserved structure. FIG. 2B shows proposed Rep-catalyzed nicking and ligating activities in a wild-type left ITR (SEQ ID NO: 53), including the T-shaped stem-loop structure of the wild-type left ITR of AAV2 with identification of A-A′ arm, B-B′ arm, C-C′ arm, two Rep Binding sites (RBE and RBE′) and also shows the terminal resolution site (trs), and the D and D′ region comprising several transcription factor binding sites and other conserved structure.

FIG. 3A provides the primary structure (polynucleotide sequence) (left) and the secondary structure (right) of the RBE-containing portions of the A-A′ arm, and the C-C′ and B-B′ arm of the wild type left AAV2 ITR (SEQ ID NO: 54). FIG. 3B shows an exemplary mutated ITR (also referred to as a modified ITR) sequence for the left ITR. Shown is the primary structure (left) and the predicted secondary structure (right) of the RBE portion of the A-A′ arm, the C arm and B-B′ arm of an exemplary mutated left ITR (ITR-1, left) (SEQ ID NO: 113). FIG. 3C shows the primary structure (left) and the secondary structure (right) of the RBE-containing portion of the A-A′ loop, and the B-B′ and C-C′ arms of wild type right AAV2 ITR (SEQ ID NO: 55). FIG. 3D shows an exemplary right modified ITR. Shown is the primary structure (left) and the predicted secondary structure (right) of the RBE containing portion of the A-A′ arm, the B-B′ and the C arm of an exemplary mutant right ITR (ITR-1, right) (SEQ ID NO: 114). Any combination of left and right ITR (e.g., AAV2 ITRs or other viral serotype or synthetic ITRs) can be used as taught herein. Each of FIGS. 3A-3D polynucleotide sequences refer to the sequence used in the plasmid or bacmid/baculovirus genome used to produce the ceDNA as described herein. Also included in each of FIGS. 3A-3D are corresponding ceDNA secondary structures inferred from the ceDNA vector configurations in the plasmid or bacmid/baculovirus genome and the predicted Gibbs free energy values.

FIG. 4A is a schematic illustrating an upstream process for making baculovirus infected insect cells (BIICs) that are useful in the production of a ceDNA vector for insertion of a transgene at a GSH loci as disclosed herein in the process described in the schematic in FIG. 4B. FIG. 4B is a schematic of an exemplary method of ceDNA production and FIG. 4C illustrates a biochemical method and process to confirm ceDNA vector production. FIG. 4D and FIG. 4E are schematic illustrations describing a process for identifying the presence of ceDNA in DNA harvested from cell pellets obtained during the ceDNA production processes in FIG. 4B. FIG. 4D shows schematic expected bands for an exemplary ceDNA either left uncut or digested with a restriction endonuclease and then subjected to electrophoresis on either a native gel or a denaturing gel. The leftmost schematic is a native gel, and shows multiple bands suggesting that in its duplex and uncut form ceDNA exists in at least monomeric and dimeric states, visible as a faster-migrating smaller monomer and a slower-migrating dimer that is twice the size of the monomer. The schematic second from the left shows that when ceDNA is cut with a restriction endonuclease, the original bands are gone and faster-migrating (e.g., smaller) bands appear, corresponding to the expected fragment sizes remaining after the cleavage. Under denaturing conditions, the original duplex DNA is single-stranded and migrates as a species twice as large as observed on native gel because the complementary strands are covalently linked. Thus in the second schematic from the right, the digested ceDNA shows a similar banding distribution to that observed on native gel, but the bands migrate as fragments twice the size of their native gel counterparts. The rightmost schematic shows that uncut ceDNA under denaturing conditions migrates as a single-stranded open circle, and thus the observed bands are twice the size of those observed under native conditions where the circle is not open. In this figure “kb” is used to indicate relative size of nucleotide molecules based, depending on context, on either nucleotide chain length (e.g., for the single stranded molecules observed in denaturing conditions) or number of basepairs (e.g., for the double-stranded molecules observed in native conditions). FIG. 4E shows DNA having a non-continuous structure. The ceDNA can be cut by a restriction endonuclease, having a single recognition site on the ceDNA vector, and generate two DNA fragments with different sizes (1 kb and 2 kb) in both neutral and denaturing conditions. FIG. 4E also shows a ceDNA having a linear and continuous structure. The ceDNA vector can be cut by the restriction endonuclease, and generate two DNA fragments that migrate as 1 kb and 2 kb in neutral conditions, but in denaturing conditions, the stands remain connected and produce single strands that migrate as 2 kb and 4 kb.

FIG. 5 is an exemplary picture of a denaturing gel running examples of ceDNA vectors with (+) or without (−) digestion with endonucleases (EcoRI for ceDNA construct 1 and 2; BamH1 for ceDNA construct 3 and 4; SpeI for ceDNA construct 5 and 6; and XhoI for ceDNA construct 7 and 8) Constructs 1-8 are described in Example 1 of International Application PCT PCT/US18/49996, which is incorporated herein in its entirety by reference. Sizes of bands highlighted with an asterisk were determined and provided on the bottom of the picture.

FIG. 6 is a schematic representation of the PAX5 gene located on Chromosome 9: 36,833,275-37,034,185 reverse strand (GRCh38:CM000671.2), and neighboring/surrounding genes or RNA sequences, such as those listed in Table 1A.

FIG. 7 is a schematic illustration depicting how an exemplary ceDNA vector comprising 5′ homology arms (HA-L) and a 3′ homology arm (HA-R) inserts a transgene into a GSH loci in the genome of a host cell. FIG. 7 shows an exemplary ceDNA vector comprising a 5′ and 3′ ITR which flank a 5′ homology arm (HA-L) and 3′ homology arm (HA-R), where the HA-L and HA-R flank a transgene expression cassette. The transgene cassette comprises an optional exemplary reporter molecule (e.g., GFP). FIG. 7 also shows how the homology arms undergo homologous recombination at the GSH loci to insert the transgene into the genome of the host's cell. The 5′ ITR and 3′ ITR can be asymmetric, symmetric or substantially symmetrical relative to one another, as described herein.

FIG. 8 is another schematic illustration depicting how an exemplary ceDNA vector comprising 5′ homology arms (HA-L) and a 3′ homology arm (HA-R) inserts a transgene into a GSH loci in the genome of a host cell. FIG. 8 shows an exemplary all-in-one ceDNA vector comprising a 5′ and 3′ ITR which flank a gene editing cassette, and a 5′ homology arm (HA-L) and 3′ homology arm (HA-R), where the HA-L and HA-R flank a transgene expression cassette. The transgene cassette comprises an optional exemplary reporter molecule (e.g., GFP). The gene editing cassette can comprise one or more of: a sgRNA expression unit and/or a nuclease expressing unit, where the nuclease expressing unit comprises one or more gene editing molecule, an enhancer (Enh), a promoter (pro), an intron (e.g., synthetic or natural occurring intron with splice donor and acceptor seq), nuclear localization signal (NLS) upstream of a nuclease (e.g., nucleic acid with an ORF encoding a Cas9, ZFN, Talen, or other endonuclease sequences). The sgRNA expression unit is enlarged to show in more detail a promoter, e.g., U6 promoter (arrow) drives the expression of 4 sgRNAs. The nuclease expressing unit is also enlarged. Transport of the nuclease expressing unit to the nuclei can be increased or improved by using a nuclear localization signal (NLS) fused into the 5′ or 3′ enzyme peptide sequence (e.g., the nuclease expressing unit, such as Cas9, ZFN, TALEN etc.). FIG. 8 also shows how the homology arms undergo homologous recombination at the GSH loci to insert the transgene into the genome of the host's cell. The 5′ and 3′ ITRs can be asymmetric, symmetric or substantially symmetrical relative to one another, as described herein.

FIG. 9A-9D show exemplary ceDNA vectors for insertion of a transgene at a GSH loci. The ITRs flank a transgene expression cassette (e.g., at least one transgene and any one or more regulatory sequences (e.g., promoters, regulatory switches, WPRE element, polyA sequences, enhancers etc.) and can comprise one or both 5′ HA (HA-L) and/or 3′ HA (HA-R) specific to the GSH regions as disclosed herein in Table 1A or 1B. FIG. 9A shows a ceDNA vector with a transgene expression cassette with an open reading frame (ORF) flanked with 5′ and 3′ homology arms that hybridize to a GSH locus identified in Tabled 1A-1B and therefore drive expression of the transgene under the endogenous promoter for the gene located in the GSH. FIG. 9B shows a ceDNA vector similar to that in FIG. 8A, except that it does not comprise a HA-R. FIG. 9C shows a ceDNA vector similar to that in FIG. 8A, except that it does not comprise a HA-L. A ceDNA vector comprising a nuclease expressing unit can be delivered in trans, such a ceDNA vector encoding a gene editing molecule, e.g., a Cas9, zinc-finger nucleases (ZFN), transcription activator-like effector nucleases (TALEN), mutated “nickase” endonuclease, class II CRISPR/Cas system (CPF1) to the ceDNA vectors of FIG. 8A-8C. Alternatively, FIG. 9D shows ceDNA vectors similar to those in FIGS. 9A-9C, except also comprising a gene editing cassette upstream of the HA-L and downstream of the 5′ ITR. Gene editing cassettes are described in FIG. 8 and. 10.

FIG. 10 is a schematic illustration of an exemplary all-in-one ceDNA vector for insertion at a GSH loci as disclosed herein. Shown in FIG. 10 is an exemplary ceDNA vector, where located between the 5′ ITR and 3′ITR is a gene editing cassette, where the gene editing cassette can comprise one or more of: a gene editing molecule (e.g., one or more sgRNA sequences), an Enh: enhancer (Enh), promoter (promoter), intron (e.g., synthetic or natural occurring intron with splice donor and acceptor seq), nuclear localization signal (NLS), a nuclease, (with an ORF for Cas9, ZFN, Talen, or other endonuclease sequences). The filled arrows represent the sgRNA seq. (single guide-RNA target sequences (e.g., 4) are selected using freely available software/algorithm picked out and validated experimentally), open arrows represent alternative sgRNA sequences. Downstream of the gene editing cassette is the 5′ HA (HA-L) and 3′ HA (HA-R), that target a GSH loci shown in Table 1A or Table 1B, and located between the HA-L and HA-R is the expression cassette to be inserted, that comprises a transgene, and in some embodiments, a promoter and/or regulatory switch as described herein. The sgRNA target a region of the HA-L. The ceDNA vector in FIG. 10 includes a Pol III promoter driven (such as U6 and H1) sgRNA expressing unit with optional orientation with respect to the transcription direction. An sgRNA target sequence for a “double mutant nickase” is optionally provided to release torsion downstream of the 3′ homology arm close to the mutant ITR. Such embodiments increase annealing and promote HDR frequency.

FIG. 11. is a schematic illustration of an exemplary ceDNA vector in accordance with the present disclosure. Three exemplary ceDNA vectors comprise a 5′ and 3′ ITRs which flank GSH 5′ and 3′ homology arms and can comprise a promoter-less transgenes suitable for insertion into GSH loci identified herein or shown in Tables 1A or 1B. In another embodiment, a ceDNA vector with 5′ and 3′ homology arms that comprises a promoter driven transgene, that can be inserted into a safe harbor site listed in Tables 1A or 1B.

FIG. 12 shows Table 11 listing exemplary genes for transgenes or GOI to be inserted into a GSH as disclosed herein.

### DETAILED DESCRIPTION

The technology described herein relates to methods, compositions and in silco screening approaches for identifying, characterizing and validating genomic safe harbor (GSH) loci in mammalian, including human genomes. Embodiments of the invention also relate to method to identify the GSH, methods to validate the GSH, and a non-viral, capsid free closed ended DNA (ceDNA) vector useful for insertion of a GOI or transgene into a GSH as identified using the methods disclosed herein. In some embodiments such a ceDNA vector comprises two ITRs, which can be asymmetrical or symmetrical, or substantially symmetrical relative to each other, where the two ITRs flank a left homology arm (HA-L) and a right homology arm (HA-R), where located between the HA-L and the HA-R is at least one heterologous nucleotide sequence (e.g., GOI or transgene. Accordingly, in some embodiments, the ceDNA vector comprises nucleic acids that are complementary to regions of the GSH that guide homologous recombination with regions of the GSH, as well as cells, kits and transgenic animals comprising the ceDNA vectors and/or transgenes inserted into the GSH using the ceDNA vectors disclosed herein.

### I Methods to Identify Genomic Safe Harbors

Screening assays, including in silico approaches have been used to identify genomic safe harbor loci in mammalian genomes, including human genomes, where methodological principles for selecting and validating GSHs have been used, including use of any of: bioinformatics, expression arrays and transcriptome analysese (e.g., RNAseq) to query nearby genes, in vitro expression assays of inserted genes into the GSH, in vitro-directed differentiation or in vivo reconstitution assays, in vitro and in xenogeneic transplant models, transgenesis in syntenic regions and analyses of patient and non-human genomic databases from individuals harboring integrated provirus sequences.

The technology described herein relates to ceDNA vectors for insertion of a transgene into a specific genomic safe harbor (GSH) region disclosed herein, and relates to use of such ceDNA vectors in methods and compositions for treating a subject with a disease, as well as for generation of cells, and/or transgenic mice or animal models in methods to validate such genomic safe harbors (GSHs).

GSHs are intragenic, intergenic, or extragenic regions of the human and mouse species genomes that are able to accommodate the predictable expression of newly integrated DNA without significant adverse effects on the host cell or organism. While not being limited to theory, a useful safe harbor must permit sufficient transgene expression to yield desired levels of the vector-encoded protein or non-coding RNA. A GSH also should not predispose cells to malignant transformation nor significantly alter normal cellular functions. What distinguishes a GSH from a fortuitous good integration event is the predictability of outcome, which is based on prior knowledge and validation of the GSH.

The discovery and validation of GSHs in the human genome will ultimately benefit human cell engineering and especially stem cell and gene therapy, and validation of true GSHs is important enabling safe clinical development and advancement of technologies and tools for targeted integration at a GSH loci, including targeting the GSH with nucleases specific for the safe harbor genes such that the transgene construct is inserted for example, by either homology direct repair (HDR) or non-homologous end-joining (NHEJ)-driven processes, where such technologies have preceded the identification of appropriate target sites.

The identification of genomic safe harbors (GSHs) was based on provirus insertions in germlines of related species within a taxonomic rank. Evolutionary conserved heritable endogenous virus elements (EVEs) was used to effectively denote genomic loci that are tolerant of insertions in the germline. Species within a taxonomic rank that with an EVE sequence at the same genomic locus confirm infection of an individual animal that was the common ancestor to species that radiated into the individual, thus defining that lineage as an EVE-positive Glade. The persistence of the EVE allele(s) through multiple epochs of the Cenozoic Era can be attributed to a single individual infected with the virus either a population bottleneck or that the EVE provided a positive selective advantage (or less likely resulted from a random integration event into a benign locus resulting in neutrality, i.e., neither acts positively nor negatively, thereby is neutral and provides no selection benefits either way. However, the probability of stabilizing an allele within a population is influenced by (i) Fitness conferred and (ii) the effective population of the species, i.e., the population of breeding animals within the group.

Comparative genomic approaches was also used to identify genomic safe harbors. In particular, GSH loci in a mammalian genome was identified by comparing interspecific introns of collinearly organized and/or synteny organized genes to identify an enlarged intron in one species relative to another species, where the enlarged intron identifies a potential genomic safe. GSH loci in a mammalian genome was also identified by comparing the intergenic distance (or space) between selected genes or adjacent genes of collinearly organized or synteny organized genes in different species to identify large variations in the intergenic spaces between the two selected genes in different species, and a potential genomic safe harbor was identified where there was a large variation in the intergenic space.

Accordingly, the disclosure herein relates to ceDNA vectors comprising nucleic acid sequences, e.g., at least one GSH-homology arm (e.g., a 5′ GSH-HA, and/or a 3′GSH-HA) and/or a guide RNA (gRNA) or guide DNA (gDNA) that target a GSH locus identified and disclosed herein, e.g., PAX5 GSH locus, a KIF6 GSH locus or any GSH loci listed in Table 1A or Table 1B. In some embodiments, the ceDNA vectors can be used to validate one or more GSH loci disclosed herein, e.g., validate the GSH loci in a mammalian genome, including a human genome. Other aspects of the technology relate to using the ceDNA vectors to modify one or more GSH loci disclosed herein, and/or ceDNA vectors that comprise GSH intermediates, e.g., a GSH that has been modified to comprise a multiple cloning site (MCS), or the like for insertion of a transgene at the identified GSH loci. GSH intermediates also refer to cells with partial recombination (i.e., where the site is nicked and recombined partially with a transgene to be inserted).

### A. Identifying Genomic Safe Harbors Using EVEs of Proto-Species or Related Species in a Taxonomic Order.

Evolutionary biology was used to identify AAV- and parvovirus or provirus remnants, referred to as endogenous virus elements (EVEs), in related species within a taxonomic rank. The results described herein demonstrate that EVEs can be acquired into the germline of a usually extinct proto-species prior to the radiation of the species, such that all evolved or descendent species retain the EVE allele. Whereas closely related species that evolved or radiated prior to the “endogenization” event remain with an empty loci. That is, the speciation occurred subsequent to EVE acquisition are therefore is monophyletic. As an illustrative example only, the locus occupied by intergenic EVE in the Macropodidae (kangaroos and related species) is identifiable in other marsupials, including Didelphis virgiana (North American opossum). These unoccupied loci are identifiable in other taxonomic families and although the EVE open reading frames are disrupted, the virus sequence represents foreign DNA inserted into the genome of totipotent germ cells, thus identifying candidate genomic safe-harbor loci.

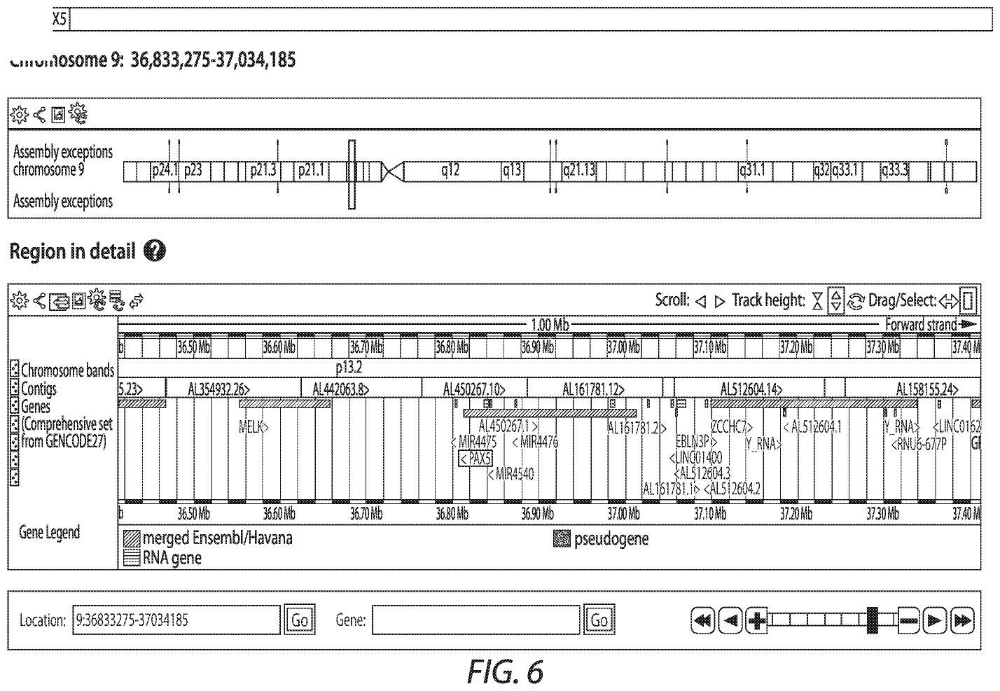

Interspecific synteny was used to identify orthologous safe-harbors in the murine and human genomes with potential usefulness in genome editing techniques, such as with mega-nucleases or CRISPR/Cas9 approaches. For example, all Cetacea have an intronic AAV EVE in the PAX5 gene. PAX5 gene (also known as “B-cell lineage specific activator” or BSAP). The homeodomain transcription factor, PAX5 is conserved in vertebrates, for example, human, chimp, macaque, mouse, rat, dog, horse, cow, pig, opossum, platypus, chicken, lizard, xenopus, C. elegans, drosphila and zebrafish. In humans, the PAX5 gene is located on human chromosome 9 at positions: 36,833,275-37,034,185 reverse strand (GRCh38:CM000671.2) or 36,833,272-37,034,182 in GRCh37 coordinates (see FIG. 6), also referred to as 9p13.2.

The EVE locus, e.g., the PAX5 gene was assessed to determine if it was a safe-harbor by inserting a reporter gene into the orthologous region in human progenitor cells. To characterize and validate a PAX4 GSH locus, a ceDNA vector as disclosed herein can be used to insert a transgene into the PAX GSH locus identified herein in cells, e.g., into mouse and human lymphomyeloid stem cells, which can be manipulated ex vivo and then engrafted into immune-cell depleted mice. The lymphomyeloid repopulate the lineages which are easily characterized with cell surface markers. Transgenic mice can also be used to test of the breadth of the safe-harbor into other tissues and systems.

The GSH loci in mammalian genomes were identified using an initial sequencing and/or in silico analysis of the sequence of genomic DNA inferred from a proto-species by multiple species within a taxonomic rank to identify endogenous virus element (EVE) or provirus nucleic acid insertions in the genomic DNA.

Methods to identify genomic safe harbor (GSH) regions in a mammalian genome were used, which comprised (a) identifying the loci of the endogenous virus element (EVE) in the genomes of related species within taxonomic rank; (b) identifying the interspecific conserved loci in the human or mouse genome based on gene conservation or synteny; and functional validation of the candidate loci as a genomic safe harbor (GSH), e.g., functional validation in human and mouse progenitor and somatic cells (e.g., any of satellite cells, airway epithelial cells, any stem cells, induced pluripotent stem cells, and the like) using at least one or more in vitro or in vivo assays as disclosed herein. In some embodiments, functional validation of the candidate loci as a genomic safe harbor can be assessed using the ceDNA vectors as disclosed herein in germline cells only in animal models and mice models at least one or more in vitro or in vivo assays as disclosed herein.

In some embodiments, the ceDNA vectors as disclosed herein can be used in functional selected from any one or more of: (a) insertion of a marker gene into the loci in human cells and measure marker gene expression in vitro; (b) insertion of marker gene into orthologous loci in progenitor cells or stem cells and engraft the cells into immune-depleted mice and/or assess marker gene expression in all developmental lineages; (c) insertion of the marker gene into the GSH of undifferentiated hematopoietic CD34+ cells followed by applying cytokines to induce differentiation into terminally differentiated cell types, wherein the hematopoietic CD34+ cells have a marker gene inserted into the candidate GSH loci; or (d) generate transgenic knock-in mouse wherein the genomic DNA of the mouse has a marker gene inserted in the candidate GSH loci, wherein the marker gene is operatively linked to a tissue specific or inducible promoter.

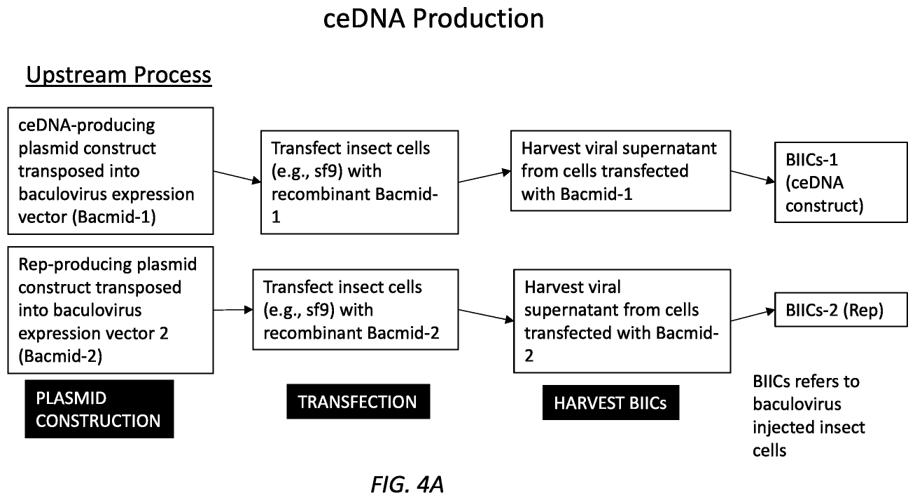

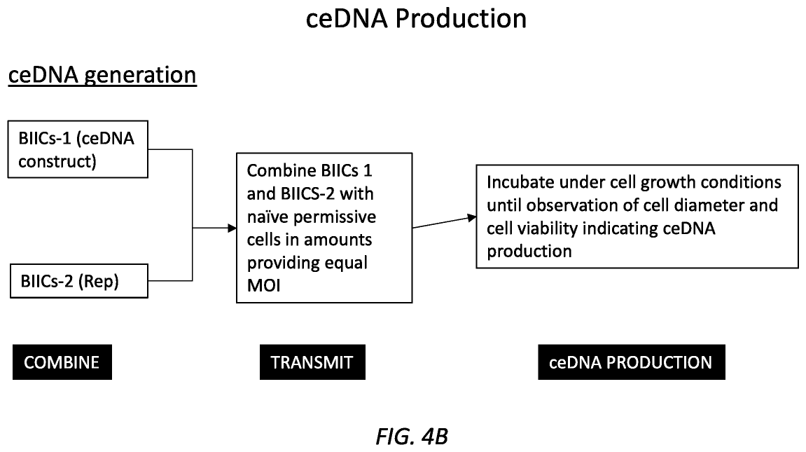

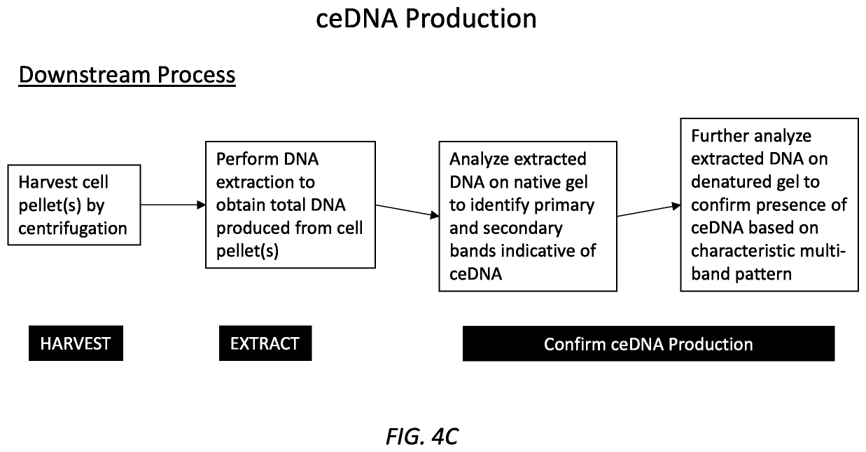

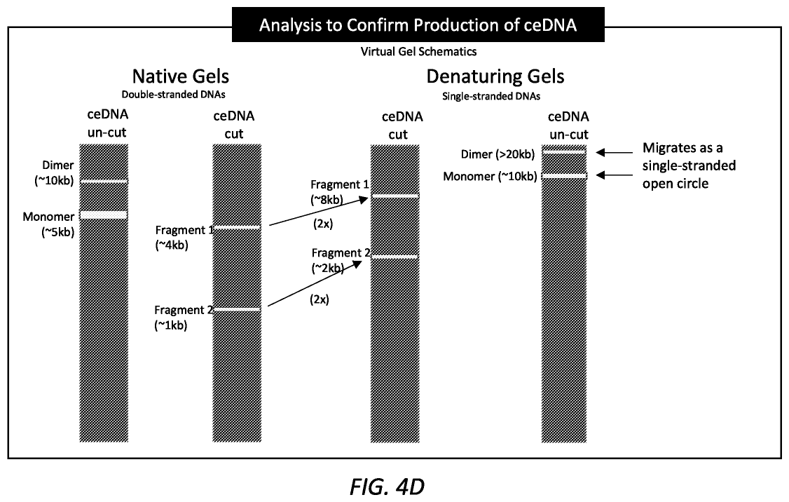

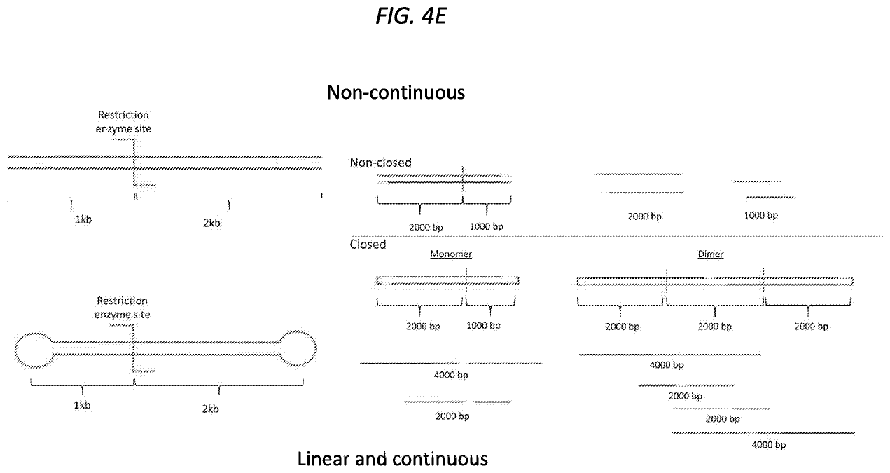

GSH loci for use in the ceDNA vectors as disclosed herein were also identified by analysis of the genome sequence of a model species for the presence of the EVE. The model species can be from any phylogenetic taxa including, but not limited to: catacea, chiroptera, Lagomorpha, Macropodidae. Other model species can be assessed, for example, rodentia, primates (except humans), monotremata. Other species can be used, for example, as listed in FIG. 4A, 4B of Lui et al., J Virology 2011; 9863-9876 which is incorporated herein in its entirety by reference. The EVE assessed is a nucleic acid comprising intronic or exonic or intergenic viral nucleic acid, viral DNA, viral DNA or DNA copies of viral RNA. In some embodiments, the EVE comprises a region of viral nucleic acid from a non-retrovirus, i.e., the viral nucleic acid is non-retroviral viral nucleic acid.

In some embodiments, the EVE is a provirus, which is the virus genome integrated into the DNA of a non-virus host cell. In some embodiments, the EVE is a portion or fragment of the virus genome. In some embodiments, the EVE is a provirus from a retrovirus. In some embodiments, the EVE is not from a retrovirus. In some embodiments, the EVE is a provirus or fragment of a viral genome from a non-retrovirus.

In some embodiments, the EVE is nucleic acid from a parvovirus. The parvovirus family contains two subfamilies; Parvovirinae, which infect vertebrate hosts and Densovirinae, which infect invertebrate hosts. Each subfamily has been subdivided into several genera. In some embodiments, the EVE is a nucleic acid from a Densovirinae, from any of the following genus, densovirus, iteravirus, and contravirus.

In some embodiments, the EVE is a nucleic acid from a parvovirinae, from any of the following genera; Parvovirus, Erythrovirus, Dependovirus.

In some embodiments, the EVE is from the subfamily of Parvovirinae include the following genera:

a. Genus Amdoparvovirus: type species: Carnivore amdoparvovirus 1. Genus includes 2 recognized species, infecting mink and fox
b. Genus Aveparvovirus: type species: Galliform aveparvovirus 1. Genus includes a single species, infecting turkeys and chickens
c. Genus Bocaparvovirus: type species: Ungulate bocaparvovirus 1. Genus includes 12 recognized species, infecting mammals from multiple orders, including primates
d. Genus Copiparvovirus: type species: Ungulate copiparvovirus 1. Genus includes 2 recognized species, infecting pigs and cows
e. Genus Dependoparvovirus: type species: Adeno-associated dependoparvovirus A. Genus includes 7 recognized species, infecting mammals, birds or reptiles
f. Genus Erythroparvovirus: type species: Primate erythroparvovirus 1. Genus includes 6 recognized species, infecting mammals, specifically primates, chipmunk or cows
g. Genus Protoparvovirus: type species: Rodent protoparvovirus 1. Genus includes 5 recognized species, infecting mammals from multiple orders, including primates
h. Genus Tetraparvovirus: type species: Primate tetraparvovirus 1. Genus includes 6 recognized species, infecting primates, bats, pigs, cows and sheep

The Parvovirus subfamily is associated with mainly warm-blooded animal hosts. Of these, the RA-1 virus of the parvovirus genus, the B19 virus of the erythrovirus genus, and the adeno-associated viruses (AAV) 1-9 of the dependovirus genus are human viruses. In some embodiments, the EVE is from a virus that can infect humans, which are recognized in 5 genera: Bocaparvovirus (human bocavirus 1-4, HboV1-4), Dependoparvovirus (adeno-associated virus; at least 12 serotypes have been identified), Erythroparvovirus (parvovirus B19, B19), Protoparvovirus (Bufavirus 1-2, BuV1-2) and Tetraparvovirus (human parvovirus 4 G1-3, PARV4 G1-3).

In some embodiments, the EVE is from a parvovirus, and in some embodiments the EVE is nucleic acid from an AAV (adeno-associated virus). Adeno-associated virus (AAV), a member of the Parvovirus family, is a small nonenveloped, icosahedral virus with single-stranded linear DNA genomes of 4.7 kilobases (kb) to 6 kb. AAV is assigned to the genus, Dependoparvovirus, because the virus was discovered as a contaminant in purified adenovirus stocks, was originally designated as adenovirus associated (or satellite) virus. AAV's life cycle includes a latent phase at which AAV genomes, after infection, may integrate into a host cells chromosomal DNA frequently at a defined locus, such as, e.g., AAVS1, and a lytic phase in which cells are co-infected with either adenovirus or herpes simplex virus and AAV, or superinfecting latent infected cells, the integrated genomes are subsequently rescued, replicated, and packaged into infectious viruses. Based on serological surveillance analyses, exposure to AAV is highly prevalent in humans and other primates and several serotypes have been isolated from various tissue samples. Serotypes 2, 3, and 6 were discovered in cultured human cells, and AAV5 was isolated from a clinical specimen, whereas AAV serotypes 1, 4, and 7-11 were isolated from nonhuman primate (NHP) tissue samples or cells. As of 2006 there have been 11 AAV serotypes described. Weitzman, et al., (2011). “Adeno-Associated Virus Biology”. In Snyder, R. O.; Moullier, P. Adeno-associated virus methods and protocols. Totowa, N.J.: Humana Press. ISBN 978-1-61779-370-7; Mori S, et al., (2004). “Two novel adeno-associated viruses from cynomolgus monkey: pseudotyping characterization of capsid protein”. Virology. 330 (2): 375-83).

In some embodiments, the EVE is a nucleic acid sequence, or part of a nucleic acid from any of the parvoviruses listed in Table 2 or Table 3A or Table 3B.

TABLE 2
Shows Endogenous viral elements (EVE) related to single stranded DNA viruses (reproduced
from Supplemental Table S6 from Katzourakis A, Gifford RJ (2010) Endogenous Viral Elements in Animal
Genomes. PLoS Genet 6(11): e1001191, which is incorporated herein in its entirety by reference).

| | | | | | | | | |
|---|---|---|---|---|---|---|---|---|
| | | | | Best viral | NR | PFAM | Genomic | Element |
| Host species ¹ | Contig ² | Location ³ | ⁴ | match ⁵ | e-value ⁶ | e-value ⁷ | region ⁸ | name ⁹ |

Parvoviridae

| Genus Dependovirus | | | | | | | AAV2 | |
|---|---|---|---|---|---|---|---|---|
| Domestic dog | NC_006619 | 12272147- | − | DQ335246 | 3.00E−36 | 4.50E−33 | 4045- | |
| (Canis familiaris) | | 12272509 | | | | | 4356 | |
| | NC_006621 | 74798635- | − | EU583391 | 2.00E−05 | 1.10E−08 | 1323- | |
| | | 74798781 | | | | | 1469 | |
| Guinea pig (8) | AAKNO2035362 | 8370- | + | DQ335246.2 | 4.00E−168 | 1.60E−87 | 321- | |
| (Cavia porcellus) | | 9796 | | | | | 1760 | |
| | AAKNO2031205 | 114399- | + | DQ335246.2 | 2.00E−43 | 3.50E−26 | 330- | |
| | | 115225 | | | | | 1208 | |
| | AAKN02030352 | 3872- | + | AY742934 | 2.00E−42 | 3.10E−22 | 969- | |
| | | 5256 | | | | | 2637 | |
| | | 11742- | | | | | | |
| | | 12062 | | | | | | |
| | AAKN02045644 | 16301- | − | DQ335246 | 2.00E−22 | 2.70E−12 | 934- | |
| | | 19700 | | | | | 4338 | |
| | AAKN02032906 | 58198- | − | DQ196319 | 5.00E−33 | 2.10E−19 | 1206- | |
| | | 58707 | | | | | 1721 | |
| Nine-banded | AAGV020719236 | 1855- | − | AY242998 | 4.00E−74 | 3.40E−56 | 2950- | |
| armadillo | | 2469 | | | | | 3681 | |
| (Dasypus | | | | | | | | |
| novemcinctus) | | | | | | | | |
| Horse | NC 009151 | 1277165- | − | EF515837 | 5.00E−09 | 8.10E−12 | 1236- | |
| (Equus caballus) | | 1277545 | | | | | 1475 | |
| | NC 009175 | 77091065- | − | AF416726 | 2.00E−12 | 4.80E−31 | 1275- | |
| | | 77091265 | | | | | 1670 | |
| Tammar wallaby (11) | ABQO010585939 | 126- | + | AY388617 | 0 | 5.90E−123 | 330- | |
| (Macropus eugenii) | | 4049 | | | | | 4386 | |
| | ABQO010091390 | 1491- | − | U48704 | 2.00E−61 | 1.80E−25 | 3604- | |
| | | 2329 | | | | | 4410 | |
| | ABQO010903052 | 518- | + | FJ688147 | 3.00E−56 | 1.80E−46 | 3037- | |
| | | 1113 | | | | | 3642 | |
| | ABQO010889914 | 572- | + | GQ368252 | 8.00E−74 | 1.90E−31 | 510- | |
| | | 1923 | | | | | 1826 | |
| | ABQO010481652 | 712- | + | AY530611 | 7.00E−40 | 1.10E−17 | 3682- | |
| | | 1284 | | | | | 4242 | |
| | ABQO010585938 | 1- | + | GQ368252 | 3.00E−17 | 2.70E−22 | 336- | |
| | | 333 | | | | | 668 | |
| | ABQO010444976 | 2723- | − | AY390557 | 4.00E−62 | 7.90E−20 | 1410- | |
| | | 3869 | | | | | 2673 | |
| | ABQO010059570 | 4449- | − | U22967 | 3.00E−25 | 3.40E−09 | 783- | |
| | | 5075 | | | | | 1532 | |
| | ABQO011172433 | 48- | − | X75093 | 3.00E−23 | 4.1e−06 ^{a} | 702- | |
| | | 525 | | | | | 1202 | |
| | ABQO010958468 | 613- | + | AY695375 | 1.00E−13 | 7.6e−12 ^{a} | 1323- | |
| | | 795 | | | | | 1505 | |
| African elephant | AAGU03013549 | 51509- | + | DQ335246 | 0 | 1.30E−112 | 330- | |
| (Loxodonta | | 53236 | | | | | 1841 | |
| Africana) | | | | | | | | |
| Mouse | NC_000069 | 12016997- | − | DQ335246 | 9.00E−68 | 6.50E−20 | 1026- | |
| (Mus musculus) | | 12020624 | | | | | 4410 | |
| | NC_000074 | 95686602- | − | AF416726 | 2.00E−09 | 9.20E−07 | 1317- | |
| | | 95687837 | | | | | 2613 | |
| | NC_000067 | 194639536- | + | J01902 | 2.00E−06 | 0.004 | 618- | EVE- |
| | | 194639781 | | | | | 881 | DV1 |
| Little brown bat | AAPE01526173 | 3215- | − | AY631965 | 0 | 6.90E−83 | 318- | |
| (Myotis | | 682 | | | | | 4410 | |
| lucifugus) | AAPE01230204 | 1592- | + | AY530577 | 1.00E−35 | 5.80E−18 | 3637- | |
| | | 1783 | | | | | 4410 | |
| | AAPE01230202 | 518- | − | AY530606 | 4.00E−13 | 6.30E−08 | 4219- | |
| | | 1284 | | | | | 4410 | |
| | AAPE01291520 | 6586- | − | DQ335246 | 2.00E−09 | 1.40E−11 | 1314- | |
| | | 6927 | | | | | 1625 | |
| Pika | AAYZ01294085 | 5975- | − | AF085716 | 1.00E−16 | 2.50E−11 | 780- | |
| (Ochotona | | 6766 | | | | | 1472 | |
| princeps) | | | | | | | | |
| Duckbilled | AAPN01125634 | 7183- | − | DQ250134 | 7.00E−12 | 1.70E−14 | 1413- | |
| platypus | | 7479 | | | | | 1715 | |
| (Ornithorhynchus | AAPN01022475 | 2333- | + | EF515837 | 4.00E−09 | 4.30E−05 | 1233- | |
| Anatinus) | | 2680 | | | | | 1583 | |
| | AAPN01206586 | 909- | + | AY530625 | 2.00E−06 | 2.60E−10 | 3046- | |
| | | 1194 | | | | | 3324 | |
| | AAPN01206585 | 357- | + | AY388617 | 4.00E−04 | 0.022 | 1389- | |
| | | 390 | | | | | 1490 | |
| European rabbit | AAGW02036031 | 4287- | + | FJ688147 | 1.00E−122 | 1.10E−53 | 354- | |
| Oryctolagus | | 7892 | | | | | 4374 | |
| cuniculus | | | | | | | | |
| Hamadyras | Contig290628- | 117545- | − | AY695376 | 0 | 1.90E−107 | 339- | |
| baboon | Contig638931 | 119924 | | | | | 2721 | |
| (Papio | Contig185865 | 216- | + | U48704 | 2.00E−67 | 0.053 | 1854- | |
| hamadryas) | | 738 | | | | | 2376 | |
| | Contig190611- | 9000- | + | AY695374 | 0 | 9.10E−99 | 321- | |
| | Contig189280 | 10344 | | | | | 1688 | |
| Cape hyrax | ABRQ01260357 | 188- | − | AY388617 | 4.00E−69 | 1.50E−28 | 396- | |
| (Procavia | | 970 | | | | | 1253 | |
| capensis) | ABRQ01135041 | 4588- | − | AY530574 | 2.00E−16 | 2.30E−07 | 4207- | |
| | | 4770 | | | | | 4389 | |
| | ABRQ01135041 | 4754- | − | AY530616 | 1.00E−10 | 0.0019 | 4030- | |
| | | 4966 | | | | | 4221 | |
| | ABRQ01135041 | 4827- | − | AY530595 | 6.00E−19 | 0.0026 | 3790- | |
| | | 5198 | | | | | 4149 | |
| | ABRQ01135041 | 5579- | − | AY530575 | 9.00E−06 | 0.0026 | 4045- | |
| | | 5848 | | | | | 4284 | |
| | ABRQ01135041 | 5998- | + | AY243026 | 2.00E−24 | 4.80E−14 | 2587- | |
| | | 6327 | | | | | 2982 | |
| Malayan flying | ABRP01003662 | 2591- | − | AY629582 | 6.00E−07 | 6.50E−11 | 1296- | |
| fox | | 2824 | | | | | 1532 | |
| (Pteropus | ABRP01170809 | 859- | − | AY629583 | 8.00E−07 | 5.10E−09 | 1287- | |
| vampyrus) | | 1059 | | | | | 1463 | |
| | ABRP01157241 | 13665- | − | DQ269987 | 7.00E−25 | 7.10E−11 | 981- | |
| | | 13959 | | | | | 1304 | |
| Brown rat | NC_005112.2 | 108702300- | + | AF513851 | 1.00E−23 | 5.30E−07 | 330- | EVE- |
| (Rattus | | 108702830 | | | | | 845 | DV1 |
| norvegicus) | NC_005101.2 | 91480723- | + | AF028704 | 8.00E−15 | 1.1e−05 ^{a} | 1011- | |
| | | 91481022 | | | | | 1328 | |
| | NC_005118.2 | 14969560- | + | AY388617 | 1.00E−07 | 0.28^{a} | 1374- | |
| | | 14969913 | | | | | 1727 | |
| | NC_005104.2 | 65632931- | + | X01457.1 | 2.00E−43 | 3.20E−31 | 2332- | |
| | | 65633263 | | | | | 2646 | |
| Bottlenose | ABRN01283281 | 1468- | + | EU253479 | 9.00E−108 | 3.60E−68 | 354- | |
| dolphin | | 3175 | | | | | 4374 | |
| (Tursiops | ABRN01191161 | 9009- | − | GQ200736 | 2.00E−07 | 4.90E−09 | 1311- | |
| truncatus) | | 9371 | | | | | 1436 | |
| Alpaca | ABRR01368792 | 4082- | + | AY530593 | 8.00E−32 | 3.80E−14 | 3997- | |
| (Vicugna pacos) | | 4485 | | | | | 4398 | |

Parvoviridae

| Genus Parvovirus | | | | | | | MVM |
|---|---|---|---|---|---|---|---|
| Guinea pig (5) | AAKN02030352 | 3872- | + | AY742934 | 8.00E−169 | 3.40E−55 | 288- |
| (Cavia porcellus) | | 5256 | | | | | 4452 |
| | | 11213- | | | | | |
| | | 13835 | | | | | |
| | AAKN02055888 | 79584- | + | AY390557 | 3.00E−64 | 4.70E−23 | 1200- |
| | | 82768 | | | | | 4413 |
| | AAKN02032906 | 58083- | − | U34253 | 3.00E−63 | 5.70E−23 | 297- |
| | | 59816 | | | | | 1862 |
| | AAKN02032908 | 10674- | + | AF036710 | 9.00E−58 | 1.40E−25 | 306- |
| | | 12353 | | | | | 1862 |
| Tenrec | AAIY01487966 | 1828- | − | AF036710 | 9.00E−45 | 1.10E−11 | 1131- |
| (Echinops | | 2527 | | | | | 1838 |
| telfairi) | | | | | | | |
| Rat | NC_005104.2 | 65636489- | − | AF036710 | 2.00E−114 | 5.40E−38 | 261- |
| (Rattus | | 65635512 | | | | | 1103 |
| norvegicus) | | 65632586- | + | | | 5.10E−143 | 2100- |
| | | 65635106 | | | | | 4557 |
| Tammar wallaby (28) | ABQO010318785 | 1- | − | FJ822038 | 8.00E−79 | 3.00E−60 | 1278- |
| (Macropus | | 1818 | | | | | 3036 |
| eugenii) | ABQO010519946 | 60- | + | AB437434 | 9.00E−84 | 7.60E−70 | 2431- |
| | | 2355 | | | | | 4527 |
| | ABQO010334457 | 1750- | + | AY684869 | 5.00E−85 | 4.50E−68 | 1719- |
| | | 4391 | | | | | 4428 |
| | ABQO010193462 | 47- | − | AY390557 | 3.00E−54 | 6.30E−64 | 3055- |
| | | 1429 | | | | | 4428 |
| | ABQO010065506 | 1048- | − | EU498687 | 2.00E−57 | 1.20E−50 | 2923- |
| | | 2591 | | | | | 4440 |
| Opossum (6) | NC_008803 | 352563141- | − | FJ592174 | 8.00E−58 | 8.80E−42 | 279- |
| (Monodelphis | | 352567160 | | | | | 4431 |
| domestica) | NC_008806 | 48166623- | + | AY684870 | 9.00E−96 | 5.10E−70 | Jun-25 |
| | | 48171573 | | | | | |
| | NC_008808 | 230386981- | + | AY390557 | 2.00E−78 | 7.20E−46 | 645- |
| | | 230396815 | | | | | 4431 |
| | NC_008806 | 113564918- | + | U34256 | 5.00E−63 | 5.10E−39 | 1338- |
| | | 352567160 | | | | | 2646 |
| Genus Amdovirus | | | | | | | AMDV |
| Cape hyrax | ABRQ01360977 | 3625- | + | X97629 | 4.00E−13 | 3.00E−19 | 2538- |
| (Procavia | | 3945 | | | | | 2855 |
| capensis) | | | | | | | |

Circoviridae

| Genus Circovirus | | | | | | | PCV-1 | |
|---|---|---|---|---|---|---|---|---|
| Domestic dog | NW_876275 | 5737517- | + | AJ298230 | 7.00E−16 | 0.00048 ^{a} | 92- | |
| (Canis familiaris) | | 5738450 | | | | | 832 | |
| | NW_876263 | 34420784- | + | AF311299 | 7.00E−07 | 0.0011 ^{a} | 647- | |
| | | 34420897 | | | | | 760 | |
| | NW_876313 | 83572- | − | DQ915950 | 2.00E−19 | 1.2e−07 ^{a} | 371- | EVE- |
| | | 84058 | | | | | 847 | CV1 |
| Cat | ACBE01536005 | 794- | + | AF311299 | 3.00E−11 | 0.0003 ^{a} | 275- | |
| (Felis cattus) | | 1486 | | | | | 826 | |
| | ACBE01511791 | 1129- | + | DQ915960 | 8.00E−10 | No | 644- | EVE- |
| | | 1325 | | | | match | 832 | CV1 |
| Giant panda | scaffold 9548* | 91- | + | GQ404844 | 7.00E−28 | 7.5e−10 ^{a} | 281- | EVE- |
| (Ailuropoda | | 741 | | | | | 919 | CV1 |
| melanoleuca) | | | | | | | | |
| Opossum | NW_001581902 | 9462550- | − | FJ623185 | 2.00E−49 | 3e−17 ^{a} | 89- | |
| (Monodelphis | | 9463357 | | | | | 982 | |
| domestica) | | | | | | | | |

¹ Common name of host species. Numbers in parentheses indicate the total number of matches identified where only a subset are shown.
² GenBank accession number of the contig containing the EVE sequence.
³ Location of EVE sequence within contig.
⁴ EVE orientation relative to contig.
⁵ Accession number and ⁶ e-value of best matching of best matching viral sequence, based on tBLASTn search against Genbank with putative EVE peptides (see methods section).
⁷ e-value of putative EVE peptide sequence to top-scoring PFAM database viral match (a removed stop codons).
⁸ Location of EVE nucleotide sequence relative to type species virus of the most closely related virus genus, based on pairwise tBLASTn with EVE peptide.
⁹ Element names are shown for elements that were orthologous across one or more host taxa (see methods section). Names follow the convention of Horie et al for Bornavirus-related elements).
Abbreviations:
AAV = adeno-associated virus;
MVM = minute virus of mice;
AMDV = Aleutian mink disease virus;
PCV-1 = porcine circovirus type-1.

TABLE 3A
List of viruses in the parvovirinae genus,
and their accession numbers

| Parvovirinae | | Accession |
|---|---|---|
| Genus | Virus species or variant | number |
| Amdoparvovirus | Aleutian mink disease virus | JN040434 |
| | Gray fox amdovirus | JN202450 |
| Aveparvovirus | Aveparvovirus Turkey | JN202450 |
| | parvovirus | |
| Bocaparvovirus | California sea lion | JN202450 |
| | bocavirus 1 | |
| | Canine bocavirus 1 | JN648103 |
| | Canine minute virus | FJ214110 |
| | Feline bocavirus | JQ692585 |
| | Human bocavirus 1 | JQ692585 |
| | Human bocavirus 4 | FJ973561 |
| | Porcine bocavirus 1 | HM053693 |
| | Porcine bocavirus 3 | JF429834 |
| | Porcine bocavirus 5 | HQ223038 |
| Copiparvovirus | Bovine parvovirus 2 | AF406966 |
| | Porcine parvovirus 4 | GQ387499 |
| Dependoparvovirus | Adeno-associated virus 1 | GQ387499 |
| | Adeno-associated virus 2 | NC_001401 |
| | Adeno-associated virus 3 | NC001729 |
| | Adeno-associated virus 3B | NC_001863 |
| | Adeno-associated virus 4 | NC_001829 |
| | Adeno-associated virus 5 | AF085716 |
| | Adeno-associated virus 6 | NC_001862 |
| | Adeno-associated virus 7 | AF513851 |
| | Adeno-associated virus 8 | AF513852 |
| | Avian-AAV ATCC VR-865 | NC_004828 |
| | Avian-AAV ATCC DA-1 | NC_006263 |
| | Bat adeno-associated virus | GU226971 |
| | California sea lion adeno- | JN420372 |
| | associated virus 1 | |
| | Bovine AAV | NC_005889 |
| | Goose parvovirus | U25749 |
| Erythroparvovirus | Erythroparvovirus Human | M13178 |
| | parvovirus B19 | |
| Protoparvovirus | Bufavirus 1 | JX027296 |
| | Canine parvovirus | M19296 |
| | Mouse parvovirus 1 | U12469 |
| | Mouse parvovirus 3 | DQ196318 |
| | Porcine parvovirus PT4 | U44978 |
| | Rat parvovirus NTU1 | AF036710 |
| Tetraparvovirus | Bovine hokovirus | EU200669 |
| | Eidolon helvum | JQ037753 |
| | parvovirus 1 | |
| | Human parvovirus 4 | AY622943 |
| | Porcine hokovirus | EU200677 |

TABLE 3B
Table 3B shows the Dependovirus sequence information.

| Taxon | Genbank | Genome | Host | Position | Size | NS | VP |
|---|---|---|---|---|---|---|---|
| AWHA01190250_ | AWHA01190250 | 3,875 | Rhinolophus | 1360:3875 | 2516 | C | P |
| Rhinolophus_ | | | ferrumequinum | | | | |
| ferrumequinum | | | | | | | |
| (horseshoe bat) | | | | | | | |
| AKZM01035630_ | AKZM01035630 | 301,611 | Ceratotherium | 19921:24311 | 4391 | C | C |
| Ceratotherium_ | | | simum | | | | |
| simum | | | | | | | |
| (white rhino) | | | | | | | |
| AWGZ01297493_ | AWGZ01297493 | 18,269 | Pteronotus | 6697:11232 | 4536 | C | C |
| Pteronotus_parnellii | | | parnellii | | | | |
| (moustached bat) | | | | | | | |
| AGTM011530899_ | AGTM011530899 | 6,551 | Daubentonia | 3508:6551 | 3044 | C | P |
| Daubentonia_ | | | madagascariensis | | | | |
| madagascariensis | | | | | | | |
| (aye-aye) | | | | | | | |
| AGTM011519523_ | AGTM011519523 | 6,189 | Daubentonia | 1:1481 | 1481 | — | P |
| Daubentonia_ | | | madagascariensis | | | | |
| madagascariensis | | | | | | | |
| AGTM010595279_ | AGTM010595279 | 402 | Daubentonia | 1:402 | 402 | — | P |
| Daubentonia_ | | | madagascariensis | | | | |
| madagascariensis | | | | | | | |
| Desmodus_ | Metagenomic * | 4894 | Desmodus | — | 4894 | C | C |
| rotundus_2 | | | rotundus | | | | |
| (vampire bat) | | | | | | | |
| JH472581_ | JH472581 | 518,716 | Tursiops | 129180:124436 | 4745 | | C |
| Tursiops_truncatus | | | truncatus | | | | |
| NW_006783413_ | NW_006783413 | 3,355,950 | Lipotes vexillifer | 1818363:1823172 | 4810 | C | C |
| Lipotes_vexillifer | | | | | | | |
| (Yangze river | | | | | | | |
| dolphin) | | | | | | | |
| KI538555_ | KI538555 | 8,596,230 | Balaenoptera | 2062073:2066503 | 4431 | C | C |
| Balaenoptera_ | | | acutorostrata | | | | |
| acutorostrata_ | | | scammoni | | | | |
| scammoni | | | | | | | |
| NW_006724242_ | NW_006724242 | 911,852 | Physeter catodon | 675028:679457 | 4430 | C | C |
| Physeter_catodon | | | | | | | |
| NW_006501254_ | NW_006501254 | 2,497,060 | Peromyscus | 2428879:2426729 | 2151 | P | P |
| Peromyscus_ | | | maniculatus | | | | |
| maniculatus_ | | | bairdii | | | | |
| bairdii | | | | | | | |
| (deer mouse) | | | | | | | |
| KE377271_ | KE377271 | 1,565,052 | Cricetulus | 1016490:1015003 | 1488 | P | P |
| Cricetulus_griseus | | | griseus | | | | |
| (Chinese hamster) | | | | | | | |
| LIPJ01023269_ | LIPJ01023269 | 148,347 | Apodemus | 15833:14683 | 1151 | P | P |
| Apodemus_ | | | sylvaticus | | | | |
| sylvaticus_ | | | | | | | |
| scaffold23294 | | | | | | | |
| (field mouse) | | | | | | | |
| AAHX01097336_ | AAHX01097336 | 23,970 | Rattus | 11263:9170 | 2094 | P | P |
| Rattus_norvegicus_ | | | norvegicus | | | | |
| chromosome19_ | | | | | | | |
| CRA_ | | | | | | | |
| 213000034410089 | | | | | | | |
| AABR07042975_ | AABR07042975 | 15,915 | Rattus | 417:2514 | 2098 | P | P |
| Rattus_norvegicus_ | | | norvegicus | | | | |
| contig_43818 | | | | | | | |

Legend:
Complete gene (F),
Partial gene (P),
* This dataset is from metagenomic study from Brazil.

In some embodiments, the EVE is nucleic acid from any serotype of AAV, including but not limited to AAV serotypes AAV1, AAV2, AAV3, AAV4, AAV5, AAV6, AAV7, AAV8, AAV9, AAV10 or AAV11 or AAV12.

In some embodiments, the EVE is a nucleic acid sequence from any of the group selected from: B19, minute virus of mice (MVM), RA-1, AAV, bufavirus, hokovirus, bocovirus, or any of the viruses listed in Table 2 or Table 3A or Table 3B, or variants thereof, that is, virus with 95%, 90%, 85%, or 80% nucleic acid or amino acid sequence identity.

In some embodiments, the EVE encodes the Rep and assembly activating non-structural (NS) proteins and structural (S) viral proteins (VP), for example, replication, capsid assembly, and capsid proteins, respectively. Such proteins include, but are not limited to, Rep (replication) proteins, including but not limited to Rep78, Rep68, Rep52, Rep40, and Cap (capsid) proteins, including but not limited to VP1, VP2 and VP3, e.g., from AAV. Structural proteins also include but are not limited to structural proteins A, B and C, for example, from AAV. In some embodiments, the EVE is a nucleic acid encoding all, or part of a non-structural (NS) protein or a structural (S) protein disclosed in Supplemental Table S2 in Francois, et al. “Discovery of parvovirus-related sequences in an unexpected broad range of animals.” Nature Scientific reports 6 (2016).

### B. Identifying Genomic Safe Harbors Using Comparative Genomic Approaches.

The identification of genomic safe harbors (GSHs) for use in the ceDNA vectors as disclosed herein was using comparative genomic approaches.

In particular, among evolutionary diverse species, the subchromosomal arrangement of genes often occur in a similar order (e.g., have collinearly) or as clustered loci (e.g., synteny). Analyzing the genomic collinearly and syntenic blocks was done to determine whether sequence/gene loss or gain occurred within that region. Disrupting the genomic organization by the addition or loss of sequences or genes suggests a degree of flexibility in that subchromosomal region without affecting viability, cellular potency, ontogeny, etc.

Accordingly, identification of GSH loci for targeting using the ceDNA vectors as disclosed herein was based on identifying provirus insertions in germlines of related species within a taxonomic rank. This approach was also applied to intergenic regions that lack coding sequences. By way of a non-limiting example, several cadherin genes are collinear in marsupial, rodent, and human species and the intergenic distance between the cadherin 8 and cadherin 11 genes are about 5.2 Mbp, 3.5 Mbp, and 2.9 Mbp, respectively. The interspecific sequence identity is limited to relatively short patches that may serve as genomic “bar-codes” to establish equivalent positions between species, within the intergenic space.

Phylogenetically, intronic sequences and spacing are more similar than intergenic sequences and spacing. Point mutations within introns are unlikely to affect genic functions except when occurring within several well characterized cis acting splicing elements within the intron, e.g., polypyrimidine tract or splice donor and acceptor signals. As a result of being embedded in genes, extensive perturbations of introns may disrupt transcript processing and translation efficiency, thus creating selective pressure for maintaining genic function.

Thus, a similar approach for identifying GSH loci useful in a ceDNA vector as disclosed herein can be applied to interspecific intron comparison, where an enlarged intron in one species relative to another species identifies a potential genomic safe harbor.

Accordingly, a ceDNA vector as disclosed herein targets a GSH loci identified using a comparison method to compare interspecific introns of collinearly organized or synteny organized genes to identify an enlarged intron in one species relative to another species. An enlarged intron is identified as being an intron that larger by at least one sigma (σ) statistical difference, or preferably, at least two sigma (σ) or more statistical difference than the same intron in the gene of different species. As an exemplary example only, in an analysis of the introns of a selected gene in three different species, e.g., human, marsupial, and rodent species (where the selected gene is collinearly organized and/or synteny organized genes between the species), if the intron is larger (i.e., longer) in one species by at least one sigma statistical difference, or at least two statistically difference as compared to the same intron in the other species, it identified an enlarged intron and a potential site as a GSH.

By way of a non-limiting an example only, if an intron “a1” of gene “A” in three different species, e.g., human, marsupial, or rodent species, is larger (i.e., longer) in one of the species by at least one sigma (σ) statistical difference or at least two sigma (σ) statistically difference, as compared to the same intron “a1” in the other species, it identifies the intron “a1” in gene “A” as enlarged intron and a potential site as a GSH.

In some embodiments, an enlarged intron is at least 20%, or at least 30%, or at least 40%, or at least 50%, or at least 60%, or at least 70%, or at least 80%, or at least 90%, or at least 100% larger, or between 20-50%, or between 50-80%, or between 80-100% larger than the comparative or corresponding intron in other species. In alternative embodiments, an enlarged intron is at least 1.2-fold, or at least about 1.4-fold, or at least about 1.5-fold, or at least about 1.6-fold, or at least about 1.8-fold, or at least about 2.0-fold, or at least about 2.2-fold, or at least about 2.4-fold, or at least about 2.5-fold or more than 2.5-fold larger (i.e., longer) than the comparative or corresponding intron in other species.

In another embodiment, a ceDNA vector as disclosed herein targets a GSH loci disclosed herein, which was identified using a method that comprises comparing the intergenic distance (or space) between selected adjacent genes of collinearly organized or synteny organized genes in different species to identify large variations in the intergenic spaces between two genes in different species, and where there is a large variation in the intergenic space, it identifies a potential genomic safe harbor. Stated differently, if there is hypervariability between the distances (e.g., intergenic spaces) between two selected genes that are collinearly organized and/or synteny organized, it identifies a potential GSH. A hypervariable region is best described in that a region between genes selected genes “A” and “B” in different species varies greatly, where genes “A” and “B” are collinearly organized and/or synteny organized between species.

As an exemplary example, a large variation in the intergenic space or distance between two selected genes is at least 20%, or at least 30%, or at least 40%, or at least 50%, or at least 60%, or at least 70%, or at least 80%, or at least 90%, or at least 100% variability between different species. In some embodiments, a large variation in the intergenic space between two selected genes of collinearly organized and/or synteny organized genes between species, or a hypervariable region between genes is identified as a region that differs in size (e.g., length) by at least one sigma (σ) statistical difference, or preferably, at least two sigma (σ) or more statistical difference in three or more different species. As an exemplary example only, in an analysis of the intergenic space between to selected genes in three different species, e.g., human, marsupial, and rodent species (where the two selected genes that are collinearly organized and/or synteny organized genes between the species), if there is variation between the size (i.e., length) between the two selected genes in one species by at least one sigma (σ) statistical difference, or at least two statistically difference as compared to the size (i.e., length) between the same genes in at least one of other species, it identifies a large variation in intergenic space and a potential site as a GSH.

By way of a non-limiting example only, if genes A, B, C, D, E are collinearly organized and/or synteny organized genes between species, if one were to compare the distance between genes D and E, and the distances between A and B in different species, and if the distances between A and B are, for example, 10 kb, 50 kb and 45 kb in three different species, and the distances between gene D and E are, e.g., 1 kb, 1.5 kb and 1.2 kb in different species, it identified the intergenic distance or space between genes A and B as hypervariable and therefore, a potential GSH. In this example, the difference between the distance between genes A and B is 5-fold (e.g., 10 kb and 50 kb), whereas the difference between genes C and D is 1.5-fold (e.g., 1 kb and 1.5 kb), and the two-tailed P value between the distance between genes A-B and genes C-D is 0.0550, thus identifying the region between gene A and B having a large variation in intergenic space and a potential region as a GSH.

Preferably, to identify a GSH locus for use in a ceDNA vector herein, one will preferably compare at least two intergenic spaces or distances between species of selected genes that are collinearly organized and/or synteny organized genes between species. For example, in the Example above, the intergenic space between genes A and B are compared with the intergenic space D and E, however, alternatively, one can compare the intergenic space between genes A and B, with the intergenic space between genes B and C etc. In some embodiments, a comparison of at least 2, or at least 3, or at least 4 intergenic spaces between genes in one will preferably compare at least two intergenic spaces that are collinearly organized and/or synteny organized between species is envisioned.

In another exemplary example, if genes A and B are collinearly organized and/or synteny organized genes between species, if one were to compare the distance between genes A and B in three or more different species (e.g., using ANOVA or other comparison methodology), and if the distance between A and B are statistically different, e.g., by at least one sigma statistical difference, or preferably, at least two sigma, in one species as compared to at least one other species, or both species, it identifies a large variation in intergenic space and a potential region as a GSH. In some embodiments, the intergenic spaces or distances between two selected genes of collinearly organized and/or synteny organized genes is assessed in at least 3, or at least 4, or at least 5, or at least 6 or at least 7 or at least 8 different species.

Accordingly, in some embodiments, a ceDNA vector as disclosed herein targets a GSH loci disclosed herein, where the GSH was identified by any of: (a) comparative genomic approaches using (i) interspecific intron comparison to identify an enlarged intron between different species of a collinearly organized or synteny organized gene and/or (ii) intergenic space comparison to identify a large variation in the intergenic spaces between adjacent genes that are collinearly organized or synteny organized; (b) identifying the enlarged intron or variant intergenic space. In some embodiments, the ceDNA vectors disclosed herein are encompassed for use in functional validation of the identified enlarge intron and/or variant intergenic space as a genomic safe harbor, e.g., functional validation in human and mouse progenitor and somatic cells (e.g., any of satellite cells, airway epithelial cells, any stem cell, induced pluripotent stem cells) using at least one or more in vitro or in vivo assays as disclosed herein. In some embodiments, the ceDNA vectors as disclosed herein can be used for functional validation of the identified enlarge intro and/or variant intergenic space as a genomic safe harbor, and can be used to assess the GSH locus in germline cells only in animal models and mice models at least one or more in vitro or in vivo assays as disclosed herein.

### C. Optional Criteria for Selecting a GSH Loci or a Nucleic Acid Region of the GSH

In some embodiments, a GSH locus for use in a ceDNA vector as disclosed herein is identified according to embodiments herein is an extragenic site that is remote from a known gene or a genomic regulatory sequence, or an intragenic site (within a gene) whose disruption is deemed to be tolerable.

In some embodiments, the GSH locus comprises may genes, including intragenic DNA comprising both intronic and extronic gene sequences as well as intergenic or extragenic material.

In some embodiments, in addition to validating the identified GSH loci using a ceDNA vector as disclosed herein, e.g., in functional in vitro and in vivo analysis as disclosed herein, a candidate GSH locus can be optionally assessed using bioinformatics, e.g., determining if the candidate GSH meets certain criteria, for example, but not limited to assessing for any one or more of the following: proximity to cancer genes or proto-oncogenes, location in a gene or location near the 5′ end of a gene, location in selected housekeeping genes, location in extragenic regions, proximity to mRNA, proximity to ultra-conserved regions and proximity to long noncoding RNAs and other such genomic regions.

By way of an example only, the previously identified GSH AAVS1 (adeno-associated virus integration site 1), was identified as the adeno-associated virus common integration site on chromosome 19 and is located in chromosome 19 (position 19q13.42) and was primarily identified as a repeatedly recovered site of integration of wild-type AAV in the genome of cultured human cell lines that have been infected with AAV in vitro. Integration in the AAVS1 locus interrupts the gene phosphatase 1 regulatory subunit 12C (PPP1R12C; also known as MBS85), which encodes a protein with a function that is not clearly delineated. The organismal consequences of disrupting one or both alleles of PPP1R12C are currently unknown. No gross abnormalities or differentiation deficits were observed in human and mouse pluripotent stem cells harboring transgenes targeted in AAVS1. Previous assessment of the AAVS1 site typically used Rep-mediated targeting which preserved the functionality of the targeted allele and maintained the expression of PPP1R12C at levels that are comparable to those in non-targeted cells. AAVS1 was also assessed and validated using ZFN-mediated recombination into iPSCs or CD34+ cells.

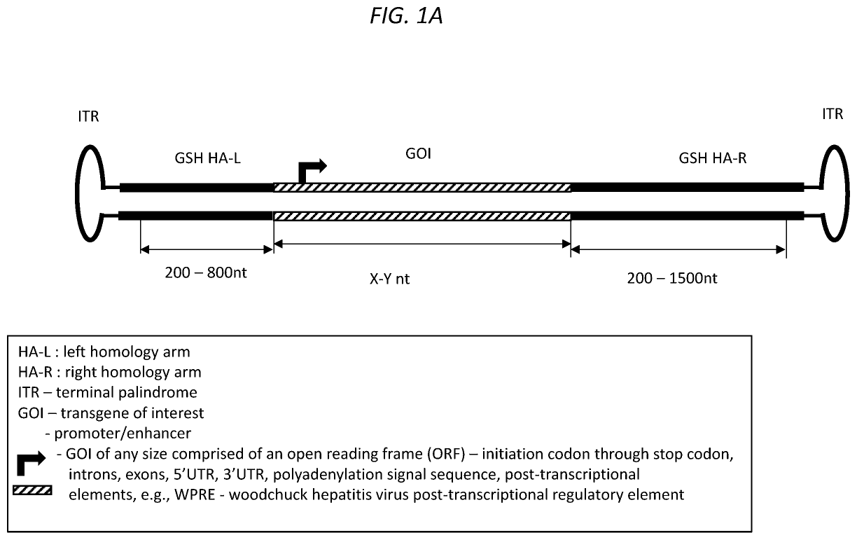

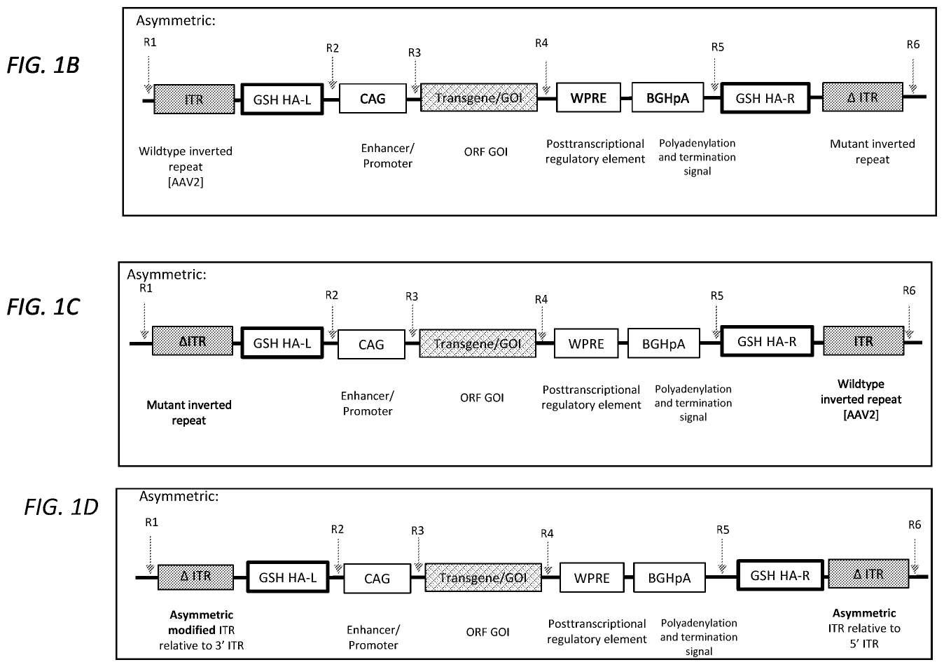

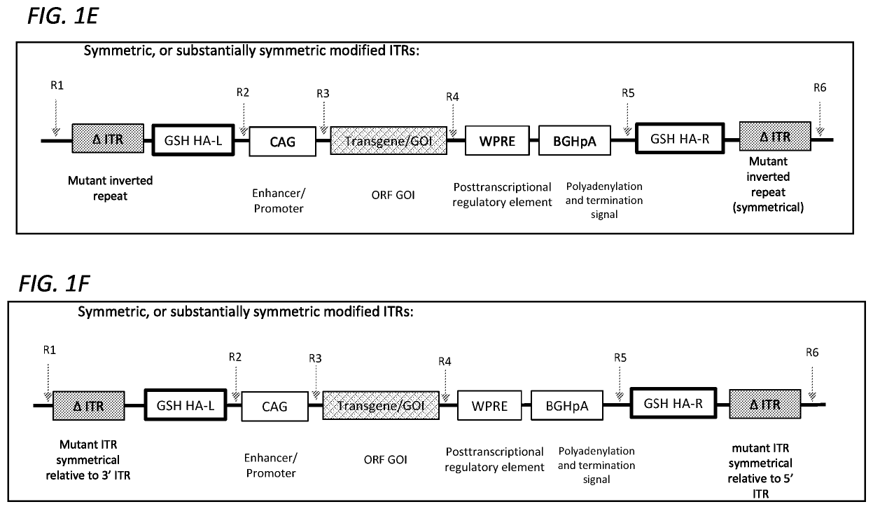

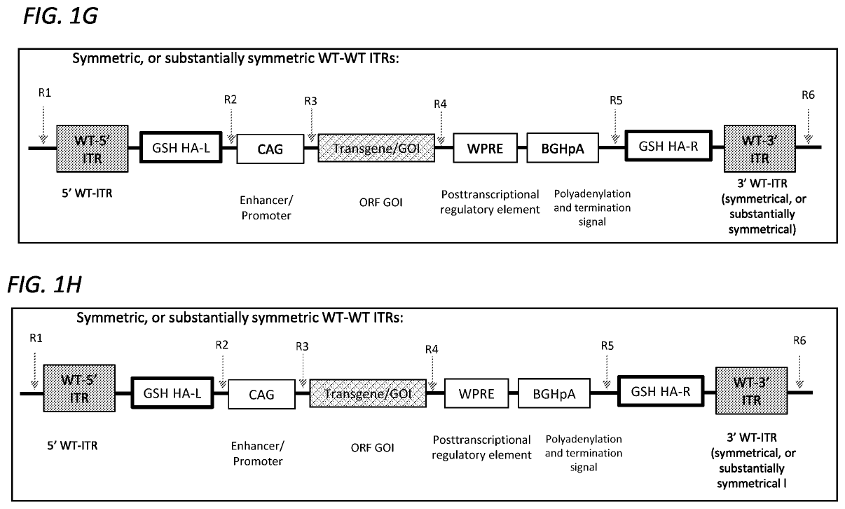

As originally characterized, the AAVS1 locus is >4 kb and is identified as chromosome 19, nucleotides 55,113,873-55,117,983 (human genome assembly GRCh38/hg38) and overlaps with exon 1 of the PPP1R12C gene that encodes protein phosphatase 1 regulatory subunit 12C. This >4 kb region is extremely G+C nucleotide content rich and is located in a particularity gene-rich region of chromosome 19 (see FIG. 1A of Sadelain et al., Nature Revs Cancer, 2012; 12; 51-58), and some integrated promoters can indeed activate or cis-activate neighboring genes, the consequence of which in different tissues is presently unknown.

AAVS1 GSH was identified by characterizing the AAV provirus structure in latently infected human cell lines with recombinant bacteriophage genomic libraries generated from latently infected clonal cell lines (Detroit 6 clone 7374 IIID5) (Kotin and Berns 1989), Kotin et al., isolated non-viral, cellular DNA flanking the provirus and used a subset of “left” and “right” flanking DNA fragments as probes to screen panels of independently derived latently infected clonal cell lines. In approximately 70% of the clonal isolates, AAV DNA was detected with the cell-specific probe (Kotin et al. 1991; Kotin et al. 1990). Sequence analysis of the pre-integration site identified near homology to a portion of the AAV inverted terminal repeat (Kotin, Linden, and Berns 1992). Although lacking the characteristic interrupted palindrome, the AAVS1 locus retained the p5 Rep proteins binding and nicking, also referred to as the terminal resolution sites (Chiorini et al. 1994; Chiorini et al. 1995; Im and Muzyczka 1989, 1990, 1992). Interestingly, the human orthologue functioned as a p5 Rep in vitro origin of DNA synthesis, thus supporting the early conjecture that AAVS1 integration is a Rep-dependent process (Kotin et al, 1990; Kotin et al, 1992; Urcelay et al. 1995; Weitzman et al. 1994). The Rep binding elements in cis were shown to be required for AAV integration and providing additional support for Rep protein involvement in the targeted, non-homolgous recombination process (Urabe, et al., Linden . . . Berns). These elements define the minimum origin of Rep-mediated DNA synthesis as the arrangement of Rep binding and nicking sites that allow RNA-primer independent strand-displacement DNA (leading strand) synthesis.

The wild-type adeno-associated virus may cause either a productive or latent infection, where the wild-type virus genome integrates frequently in the AAVS1 locus on human chromosome 19 in cultured cells (Kotin and Berns 1989; Kotin et al. 1990). This unique aspect of AAV has been exploited as one of the first so-called “safe-harbors” for iPSC genetic modification. AAVS1, as originally defined (Kotin et al., 1991) is situated on chromosome 19 between nucleotides 55,113,873-55,117,983 (human genome assembly GRCh38/hg38) and overlaps with exon 1 of the PPP1R12C gene that encodes protein phosphatase 1 regulatory subunit 12C. Interesting, PPP1R12C exon 1 5′ untranslated region contains a functional AAV origin of DNA synthesis indicated within the following sequences (Urcelay et al. 1995): The initiation methionine codon is underlined, the GCTC Rep-binding motifs and terminal resolution site (GGTTGG) are indicated with bold font: 55,117,600-TGGTGGCGGCGGTTGGGGCTCGGCGCTCGCTCGCTCGCTCGCTGGGCGGGCGGTGCGATG-55,117,540.

Surprisingly, the human chromosome 19 AAVS1 safe-harbor is within a exonic region of PPP1R12C, the gene encoding protein phosphatase regulatory 1 regulatory subunit 12C. The selection of the exonic integration site is non-obvious, and perhaps counter-intuitive, since insertion and expression of foreign DNA will likely disrupt the expression of the endogenous genes. Apparently, insertion of the AAV genome into this locus does not adversely affect cell viability or iPSC differentiation (DeKelver et al. 2010; Wang et al. 2012; Zou et al. 2011). Integration occurs by non-homologous recombination that requires the presence of AAV Rep proteins in trans and the minimum origin of AAV DNA synthesis in cis on both recombination substrates which then permits Rep-protein mediated juxtapositioning of the AAV and genomic DNAs (Weitzman et al. 1994).

The Rep-dependent minimum origin of DNA synthesis consists of the p5 Rep protein binding elements (RBE) and properly positioned terminal resolution site (trs), as exemplified by the AAV2 trs AGT|TGG and the AAV5 trs AGTG|TGG (the vertical line indicates the nicking position). In addition, the involvement of cell protein complexes has been inferred, but not yet identified or characterized.

These virus replication elements must function very efficiently or the virus would become extinct due to lack of replicative fitness, whereas, the small, non-coding, ca. 35 bp element in AAVS1 may have no function in the host. However, the AAVS1 locus has been established as a somatic cell safe harbor and disruption of the locus in totipotent or germline cells may interfere with ontogeny.

The AAVS1 locus is within the 5′ UTR of the highly conserved PPP1R12C gene. The Rep-dependent minimal origin of DNA synthesis is conserved in the 5′UTR of the human, chimpanzee, and gorilla PPP1R12C gene. However, in rodent species (mouse and rat), substitutions occur with increased frequency within the preferred terminal resolution site compared to adjacent non-coding DNA. The incidental rather than selected or acquired genotype of may affect the efficiency of the other species the specific sequences in the 5′ UTR.

In some embodiments, a ceDNA vector as disclosed herein can be used to assess a candidate GSH locus in Table 1A or 1B, where the locus is identified to meet the criteria of a GSH if it is safe and targeted gene delivery can be achieved that has limited off-target activity and minimal risk of genotoxicity, or causing insertional oncogenesis upon integration of foreign DNA, while being accessible to highly specific nucleases with minimal off-target activity.

While the GSH is validated based on in vitro and in vivo assays using ceDNA vectors as described herein, in some embodiments, additional selection can be used based on determining whether the GSH falls into a particular criterion. For example, in some embodiments, a GSH loci identified herein is located in an exon, intron or untranslated region of a dispensable gene. Analysis shows that integration sites of provirus in tumors commonly lie near the starting point of transcription, either upstream or just within the transcription unit, often within a 5′ intron. Proviruses at these locations have a tendency to dysregulate expression by increasing the rate of transcription either via promoter or via enhancer insertions. Accordingly, in some embodiments, a GSH locus identified herein is selected based on not being proximal, or with close proximity to a cancer gene. In some embodiments, a GSH does not have an integration site located near the starting point of transcription of a cancer gene, e.g. upstream or in the 5′ intron of a cancer gene or proto-oncogene. Such cancer genes are well known to one of ordinary skill in the art, and are disclosed in Table 1 in Sadelain et al., Nature Revs Cancer, 2012; 12; 51-58, which is incorporated herein in its entirety. Exemplary databases of genes implicated in cancer are well known, e.g., Atlas gene set, CAN gene sets, CIS (RTCGD) gene set, and described in Table 4 below: Table 4: Databases identifying genes implicated in cancer. *Gene lists and links to original sources are available at The Bushman lab cancer gene list website (see Further information). CAN, cancer; CIS, common insertion site; References in the last column represent the reference number in Sadelain et al., Nature Revs Cancer, 2012; 12; 51-58.

| | Number | | | |
|---|---|---|---|---|
| Gene set* | of genes | Species | Description | Refs |
| Atlas | 999 | human | This gene set is from the Atlas of genetics | 41 |
| | | | and cytogenetics in oncology and hematology. | |
| | | | It lists both hybrid genes found in at least one | |
| | | | cancer case and gene amplifications or | |
| | | | homozygous deletions found in a significant | |
| | | | subset of cases in a given cancer type | |
| Miscellaneous | 187 | Multiple | This gene set is from Retroviruses (Cold Spring | 35 |
| | | | Harbor Laboratory Press), an early version | |
| | | | of the CIS database, a list from T. Hunter, The | |
| | | | Salk Institute, La Jolla, California, | |
| | | | USA, and miscellaneous additions | |
| | | | from the scientific literature | |
| CAN genes | 192 | | This gene set includes 192 common genes that | 42 |
| | | | were mutated at significant frequency in all | |
| | | | tumors of human breast and colorectal cancers | |
| CIS | 593 | Mouse | This gene set is from the Mouse | 36 |
| (RTCGD) | | | Variation Resource and lists | |
| | | | retroviral insertional mutagenesis | |
| | | | in mouse hematopoietic tumors | |
| Human | 38 | Human | This gene set is a list of lymphoid-specific | |
| lymphoma | | | oncogenes that was compiled by M. | |
| | | | Cavazzana-Calvo and colleagues, | |
| | | | Hopital Necker, Paris, France | |
| Sanger | 452 | Human | This gene set is from the Cancer Gene | 43 |
| | | | Census, a compilation from the scientific | |
| | | | literature of “mutated genes that are | |
| | | | causally implicated in oncogenesis.” | |
| Waldman | 455 | Human | This gene set is from the Waldman gene | |
| | | | database and lists cancer | |
| | | | genes sorted by chromosomal | |
| | | | locus and includes links to OMIM | |
| AllOnco | 2,070 | Mouse | This database is a master set of the seven sets | |
| | | and | described above in which all genes are | |
| | | human | converted to their human homologues | |

In some embodiments, a GSH loci useful for being targeted by the ceDNA vectors as disclosed herein has any or more of the following properties: (i) outside a gene transcription unit; (ii) located between 5-50 kilobases (kb) away from the 5′ end of any gene; (iii) located between 5-300 kb away from cancer-related genes; (iv) located 5-300 kb away from any identified microRNA; and (v) outside ultra-conserved regions and long noncoding RNAs. In some embodiments, a GSH locus useful for being targeted by the ceDNA vectors as disclosed herein has any or more of the following properties: (i) outside a gene transcription unit; (ii) located >50 kilobases (kb) from the 5′ end of any gene; (iii) located >300 kb from cancer-related genes; (iv) located >300 kb from any identified microRNA; and (v) outside ultra-conserved regions and long noncoding RNAs. In studies of lentiviral vector integrations in transduced induced pluripotent stem cells, analysis of over 5,000 integration sites revealed that ˜17% of integrations occurred in safe harbors. The vectors that integrated into these safe harbors were able to express therapeutic levels of β-globin from their transgene without perturbing endogenous gene expression.

### II. Functional Validation of a Candidate GSH Using In Vitro and In Vivo Assays

While not being limited to theory, a useful GSH region must permit sufficient transgene expression to yield desired levels of the transgene expressed by the ceDNA (e.g., protein or non-coding RNA), and should not predispose cells to malignant transformation nor significantly negatively alter cellular functions.

Methods and compositions for validating the candidate GSH regions using the ceDNA vectors as disclosed herein include, but are not limited to; bioinformatics, in vitro gene expression assays, in vitro and in vivo expression arrays to query nearby genes, in vitro-directed differentiation or in vivo reconstitution assays in xenogeneic transplant models, transgenesis in syntenic regions and analyses of patient databases from individuals.

In one embodiment, the validation of the GSH using a ceDNA vectors as disclosed herein is useful to check that there is no germline integration of the introduced gene, reducing risks that there is germline transmission of the ceDNA gene therapy vector.

Following identification of a target loci or candidate GSH, a series of in vitro and in vivo assays using the ceDNA vectors as disclosed herein can be used to establish safety and in particular, the absence of oncogenic potential. In vitro oncogenicity assays can be based on the experience in previous gene therapy T-cell product characterizations.

### A. In Vitro Assays to Validate the GSH

In some embodiments, the GSH can be validated by a number of assays. In some embodiments, functional assays using a ceDNA vector as disclosed herein can be selected from any one or more of: (a) insertion of a marker gene into the loci in human cells and measure marker gene expression in vitro; (b) insertion of marker gene into orthologous loci in progenitor cells or stem cells and engraft the cells into immunodepleted mice and/or assess marker gene expression in all developmental lineages; (c) differentiate hematopoietic CD34+ cells into terminally differentiated cell types, wherein the hematopoietic CD34+ cells have a marker gene inserted into the candidate GSH loci; or (d) generate transgenic knock-in mouse wherein the genomic DNA of the mouse has a marker gene inserted in the candidate GSH locus, wherein the marker gene is operatively linked to a tissue specific or inducible promoter.

In some embodiments, a functional assay to validate the GSH involves using a ceDNA vector as disclosed herein for insertion of a marker gene (e.g., luciferase, e.g., SEQ ID NO: 56) into the loci of a human cell and determination of expression of the marker in vitro. In some embodiments, the marker gene is introduced by homologous recombination. In some embodiments, the marker gene is operatively linked to a promoter, for example, a constitutive promoter or an inducible promoter. The determination and quantification of gene expression of the marker gene can be performed by any method commonly known to a person of ordinary skill in the art, e.g., gene expression using e.g., RT-PCR, Affymetrix gene array, transcriptome analysis; and/or protein expression analysis (e.g., western blot) and the like. In some embodiments, the effect of the integrated marker transgene on neighboring gene expression is determined in cultured cells in vitro.

In some embodiments, the cell the marker gene is introduced into is a mammalian cell, e.g., a human cell or a mouse cell or a rat cell. In some embodiments, the cell is a cell line, e.g., a fibroblast cell line, HEK293 cells and the like. In some embodiments, the cell used in the assay are pluripotent cells, e.g., iPSCs or clonable cell types, such as T lymphocytes. In some embodiments, the gene expression of the insertion of a marker gene into a variety of different cell populations, including primary cells is assessed. In some embodiments, a iPSC that has an introduced marker gene is differentiated into multiple lineages to check consistent and reliable gene expression of the marker gene in different lineages.

In some embodiments, a ceDNA vector as disclosed herein is used to insert a marker gene into a candidate GSH loci in the genome of hematopoietic cells, such as, for example, CD34+ cells, and differentiated into different terminally differentiated cell types.

In some embodiments, a cell population that has a marker gene introduced into the candidate GSH can be assessed for possible tissue malfunction and/or transformation. For example, a CD34+ cells or iPSCs are assessed for aberrant differentiation away from normal lineage differentiation, and/or increased proliferation which would indicate a risk of cancer.

In some embodiments, the gene expression levels of proximal genes are determined. For instance, in some embodiments, if the integrated marker gene results in aberrant gene expression of surrounding or neighboring gene expression, or other dysregulation, such as a downregulation or upregulation of gene expression of the neighboring genes, the candidate loci is not selected as a suitable GSH. In some embodiments, if no change is detected in the expression level of a neighboring gene, the candidate loci is nominated, or selected, as a GSH. In some embodiments, the gene expression of flanking, proximal or neighboring genes is determined, where a proximal or neighboring gene can be within about 350 kb, or about 300 kb, or about 250 kb or about 200 kb or about 100 kb, or between 10-100 kb, or between about 1-10 kb or less than 1 kb distance (upstream or downstream) from the site of insertion of the marker gene (i.e., genes or RNA sequences flanking either in the 5′ or 3′ of the insertion loci).

In some embodiments, the epigenetic features and profile of the targeted candidate GSH loci is assessed before and after introduction of the marker gene to determine whether the introduction of the marker gene affects the epigenetic signature of the GSH, and/or surrounding or neighboring genes within about 350 kb upstream and downstream of the site of integration.

In some embodiments, insertion of a marker gene into a candidate GSH loci is assessed using a ceDNA vector as disclosed herein to see if the loci can accommodate different integrated transcription units. In some embodiments, the ceDNA vector as disclosed herein comprises a marker gene operatively linked to a range of different genetic elements, including promoters, enhancers and chromatin determinants, including locus control regions, matrix attachments regions and insulator elements) and marker gene expression is assessed, as well as, in some embodiments, the gene expression of neighboring genes within about 350 kb, or about 300 kb, or about 250 kb or about 200 kb or about 100 kb, or between 10-100 kb, or between about 1-10 kb or less than 1 kb distance (upstream or downstream) from the site of insertion of the marker gene.

In some embodiments, where a GSH loci is associated with a specific gene, the ceDNA vector as disclosed herein can be used to knock-down the gene to assess and validate that the gene is either not necessary or is dispensable. As an exemplary example, one candidate GSH is the PAX5 gene (also known as Paired Box 5, or “B-cell lineage specific activator protein” or “BSAP”). In humans PAX5 is located on chromosome 9 at 9p13.2 and has orthologues across many vertebrate species, including, human, chimp, macaque, mouse, rat, dog, horse, cow, pig, opossum, platypus, chicken, lizard, xenopus, C. elegans, drosophila and zebrafish. PAX5 gene is located at Chromosome 9: 36,833,275-37,034,185 reverse strand (GRCh38:CM000671.2) or 36,833,272-37,034,182 in GRCh37 coordinates.

PAX5 gene is surrounded by several different coding genes and RNA genes, as shown in FIG. 1. Accordingly, in one embodiment, the effect on the cell function and gene expression of neighboring cells on RNAi knockdown of PAX5 could be assessed, and where knock-down of the candidate gene in the GSH loci does not have significant effect, the gene can be identified as a GSH. Also, in vitro assays using RNAi to knock-out the GSH gene are important to determine the dispensability of the disrupted gene, especially resulting from biallelic disruption, as is often the case with endonuclease-mediated targeting.

In some embodiments, because cancer chemotherapy cytotoxic agents can have genotoxic and carcinogenic potential, standard in vitro studies for preclinical evaluations of these types of drugs can also be used. The ability of a primary T cell to grow without cytokines and cell signaling is a feature of carcinogenic transformation.

For example, in some embodiments, one can use a ceDNA vector as disclosed herein to introduce the marker gene into the candidate GSH loci of T-cells, e.g., SB-728-T cells and culture without cytokine support for several weeks and demonstrate that normal cell death occurs.

In another embodiment, the classic biological cell transformation assay is anchorage-independent growth of fibroblasts and is a stringent test of carcinogenesis. Accordingly, in some embodiments, a ceDNA vector as disclosed herein can be used to insert a marker gene into a target GSH loci in fibroblasts and assessed for anchorage-independent growth. Other in vitro assays or tests for evaluating oncogenicity can be used, e.g., mouse micronucleus test, anchorage independent growth, and mouse lymphoma TK gene mutation assay.

In some embodiments, the marker gene is selected from any of fluorescent reporter genes, e.g., GFP, RFP and the like, as well as bioluminescence reporter genes. Exemplary marker genes include, but are not limited to, glutathione-S-transferase (GST), horseradish peroxidase (HRP), chloramphenicol acetyltransferase (CAT) beta-galactosidase, beta-glucuronidase, luciferase, green fluorescent proteins (e.g., GFP, GFP-2, tagGFP, turboGFP, sfGFP, EGFP, Emerald, Azami Green, Monomeric Azami Green, CopGFP, AceGFP, ZsGreen1), HcRed, DsRed, cyan fluo-rescent protein (CFP), yellow fluorescent proteins (e.g., YFP, EYFP, Citrine, Venus YPet, PhiYFP, ZsYellow1), cyan fluorescent proteins (e.g., ECFP, Cerulean, CyPet AmCyan1, Midoriishi-Cyan) red fluorescent proteins (e.g., mKate, mKate2, mPlum, DsRed monomer, mCherry, mRFP1, DsRed-Express, DsRed2, HcRed-Tandem, HcRed1, AsRed2, eqFP611, mRaspberry, mStrawberry, Jred), orange fluorescent proteins (e.g., mOrange, mKO, Kusabira-Orange, monomeric Kusabira-Orange, mTangerine, tdTomato) and autofluorescent proteins including blue fluorescent protein (BFP).

In some embodiments, the marker gene, or reporter gene sequences include, without limitation, DNA sequences encoding β-lactamase, β-galactosidase (LacZ), alkaline phosphatase, thymidine kinase, green fluorescent protein (GFP), chloramphenicol acetyltransferase (CAT), luciferase (e.g., SEQ ID NO: 56), and others well known in the art. When associated with regulatory elements which drive their expression, the reporter sequences, provide signals detectable by conventional means, including enzymatic, radiographic, colorimetric, fluorescence or other spectrographic assays, fluorescent activating cell sorting assays and immunological assays, including enzyme linked immunosorbent assay (ELISA), radioimmunoassay (RIA) and immunohistochemistry. For example, where the marker sequence is the LacZ gene, the presence of the ceDNA vector carrying the signal is detected by assays for β-galactosidase activity. In some embodiments, where the marker gene is green fluorescent protein or luciferase, the ceDNA vector carrying the signal may be measured calorimetrically based on visible light absorbance or light production in a luminometer, respectively. Such reporters can, for example, be useful in verifying the tissue-specific targeting capabilities and tissue specific promoter regulatory activity of a nucleic acid.

In some embodiments, bioinformatics can be used to validate the GSH, for example, reviewing sequences of databases of patient-derived autologous iPSC, as described in Papapetrou et al., 2011, Na. Biotechnology, 29; 73-78, which is incorporated herein in its entirety.

Additionally, once a GSH and target integration site in GSH is identified, bioinformatics and or web-based tools can be used to identify potential off-target sites. For example, bioinformatics tools such as Predicted Report of Genome-wide Nuclease Off-Target Sites (PROGNOS, available at: world-wide web site: baolab.bme.gatech.edu/Research/BioinformaticTools/prognos.html; and CRISPOR, available at world-wide web site: crispor.tefor.net/), for designing CRISPR/Cas9 target and predicting off-target sites. CRISPOR and PROGNOS can provide a report of potential genome-wide nuclease target sites for ZFNs and TALENs. Once a particular target site is identified, the programs can provide a list of ranking potential off-target sites.

### B. In Vivo Assays to Validate the GSH

In some embodiments, ceDNA vectors as disclosed herein can be used in in vivo assays to functionally validate the GSH as well as in in vitro assays. In some embodiments, ceDNA vectors as disclosed herein can be used for in vivo evaluation of GSHs, e.g., generation of transgenic mice bearing a transgene that are integrated into syntenic regions.

In some embodiments, a ceDNA vector as disclosed herein is useful in an in vivo functional assay to validate the GSH, and involves insertion of a marker gene into the loci of a iPSC and transplantation to immunodeficient mice. In some embodiments, the insertion of a marker gene into a iPSC and the modified iPSC implanted into immunodeficient mice and assessed over a period of time. Such an in vivo assay allows any genotoxic event to be assessed, including atypical or aberrant differentiation (e.g., changes in hematopoietic transformation and/or clonal skewing of hematopoiesis), as well as the outgrowth of tumorigenic cells to be assessed from a rare event.

As such, the ceDNA vectors as disclosed herein can be used in in vivo methods in immunodeficient mice, or hematopoietic cells which are well known to one of ordinary skill in the art, and are disclosed in Zhou, et al. “Mouse transplant models for evaluating the oncogenic risk of a self-inactivating XSCID lentiviral vector.” PloS one 8.4 (2013): e62333, which is incorporated herein in its entirety by reference, where the malignancy incidence from the introduced modified hematopoietic cells or iPSC can be assessed as compared to control or cells where no marker gene is introduced at the target loci in the GSH. In some embodiments, hematopoietic malignancy can be assessed. In some embodiments, lineage distribution of peripheral blood cells in the recipient immunodeficient mice is assessed to determine myeloid skewing and a signal of insertional transformation or adverse effects due to the marker gene inserted at the GSH loci.

In some embodiments, a ceDNA vector as disclosed herein can be used in a recipient mouse strain which is immunodeficient, such that if tumors do arise in such mice, one can characterize these tumors and evaluate whether they are of human origin. If tumors are of human origin, then it will be necessary to further evaluate their clonality with respect to the insertion of the marker gene at the GSH loci or any dysregulation gene expression (upregulation or downregulation) of on- or off-target sites, such as flanking RNA sequences or genes. However, clonality observed in a marker-gene introduced cell does not necessarily equal causality and may instead be an innocent label that merely reflects the tumor's clonal origin.

In some embodiments, in vivo assays can be used that rely on the fact that human T cells can be maintained in immunodeficient NOG mice. Such an assay requires the marker gene to be introduced into the target GSH loci and modified human T cells allowed to live and expand for months in the NOG model, and compared to non-modified T cells. In some embodiments, a model with human T-cell xeno-GVHD can be used, where 2 months is allowed for a maximal time for proliferation of cells before animals died of GVHD, and defining a dose and donors that gave reliable GVHD in the NOG mice. After 2 months, the animals are euthanized and all tissues evaluated by histology for neoplasms, immunostaining to detect human cells, and gene expression analysis (e.g., Affymetrix array or RT-PCR of flanking genes surrounding the GSH insertion loci) for detection of modified gene expression of on-target and off-target sites.

In some embodiments, a ceDNA vector as disclosed herein can be used in an in vivo assay to functionally validate the candidate loci as a GSH is generating knock-in transgenic animals or transgenic mice.

Testing for Successful Gene Editing into a GSH of an iPSC or T-Lympocyte or Other Host Cell

Assays well known in the art can be used to test the efficiency of insertion of a marker gene into a GSH locus using a ceDNA vector as disclosed herein, where the ceDNA vector is used in both in vitro and in vivo models. Expression of the marker gene can be assessed by one skilled in the art by measuring mRNA and protein levels of the desired transgene (e.g., reverse transcription PCR, western blot analysis, and enzyme-linked immunosorbent assay (ELISA)). In one embodiment, the expression of the marker or reporter protein that can be used to assess the expression of the desired transgene, for example by examining the expression of the reporter protein by fluorescence microscopy or a luminescence plate reader. An exemplary reporter protein is luciferase and can be encoded by the nucleic acid sequence of SEQ ID NO: 56. For in vivo applications, protein function assays can be used to test the functionality of a given gene and/or gene product to determine if gene editing has successfully occurred. It is contemplated herein that the effects of gene editing in a cell or subject can last for at least 1 month, at least 2 months, at least 3 months, at least four months, at least 5 months, at least six months, at least 10 months, at least 12 months, at least 18 months, at least 2 years, at least 5 years, at least 10 years, at least 20 years, or can be permanent.

A GSH is where transgene insertion does not cause significant negative effects. A genomic safe harbor site in a given genome (e.g., human genome) can be determined using techniques known in the art and described in, for example, Papapetrou, ER & Schambach, A. Molecular Therapy 24(4):678-684 (2016) or Sadelain et al. Nature Reviews Cancer 12:51-58 (2012), the contents of each of which are incorporated herein by reference in their entirety.

III. ceDNA Vectors, Constructs and Kits for Targeted Homologous Recombination at a GSH Locus

As described above, nucleases specific for the safe harbor genes can be utilized such that the transgene construct is inserted by either HDR- or NHEJ-driven processes.

A. ceDNA Vectors Comprising a Portion of the GSH Locus

One aspect of the technology described herein relates to a non-viral, capsid-free DNA vector with covalently-closed ends (referred to herein as a “closed-ended DNA vector” or a “ceDNA vector”) for insertion of a transgene into a GSH region, and methods of use of such ceDNA vectors, e.g., to treat a disease. In some embodiments, a ceDNA vector comprises at least a portion of the GSH nucleic acid identified as a genomic safe harbor (GSH) in the methods described herein.

A ceDNA vector for insertion of a GOI or transgene into a GSH as described herein is described herein and in International Patent Application PCT/US18/49996, filed on Sep. 7, 2018, which is incorporated herein in its entirety by reference. In particular, a ceDNA vector useful in the methods and compositions as disclosed herein is described in International Patent Application PCT/US18/064242, filed on Dec. 6, 2018, which is incorporated herein in its entirety by reference, where the ceDNA vector is configured for gene editing and a ceDNA vector comprises a region, e.g., one or more homology arms comprising at least a portion of a GSH identified herein.

In some embodiments, a ceDNA vector useful in the methods and compositions as disclosed herein comprises a transgene for insertion at the GSH locus (e.g., an expression cassette) and at least one nucleic acid sequence that targets a GSH locus, where the nucleic acid sequence can be (i) a guide DNA (gDNA) or guide RNA (gRNA) that is specific to the GSH locus and/or the GSH-HA, or (ii) at least one GSH-specific homology arm (e.g., a 5′ GSH HA and/or a 3′ GSH HA).

In some embodiments, a ceDNA vector useful in the methods and compositions as disclosed herein comprises at least a target site of integration in a GSH, and at least a 5′ and/or 3′ portions of the GSH nucleic acid (i.e., HA-L and/or HA-R) flanking the target site of integration into the hosts cells' genome.

The ceDNA vectors, methods and compositions for insertion of a transgene into a GSH as described herein described can be used to introduce a new nucleic acid sequence into the genome of a host cell at a specific site, e.g., the safe harbor as described herein. Such methods can be referred to as “DNA knock-in systems.” The DNA knock-in system, as described herein, allows donor sequences to be inserted at a defined target site, e.g., at a GSH locus with high efficiency, making it feasible for many uses such as creation of transgenic animals expressing exogenous genes, preparing cell culture models of disease, preparing screening assay systems, modifying gene expression of engineered tissue constructs, modifying (e.g., mutating) a genomic locus, and gene editing, for example by adding an exogenous non-coding sequence (such as sequence tags or regulatory elements) into the genome. The cells and animals produced using methods provided herein can find various applications, for example as cellular therapeutics, as disease models, as research tools, and as humanized animals useful for various purposes.

The DNA knock-in systems of the present disclosure also allow for gene editing techniques using large donor sequences (<5 kb) to be inserted at defined target site, e.g., GSH locus in a genome of a host cell, thus providing gene editing of larger genes than current techniques. In some embodiments, homology arms, e.g., HA-R and HA-R as disclose herein can be, for example 50 base pairs to two thousand base pairs, provide targeted insertion of the transgene to the GSH locus with excellent efficiency (higher on-target) and excellent specificity (lower off-target), and in some embodiments, HDR can occur without the use of nucleases.

The DNA knock-in systems of the present disclosure also provide several advantages with respect to the administration of donor sequences by themselves for gene editing. First, administering ceDNA vectors as described herein within delivery particles of the present disclosure is not precluded by baseline immunity and therefore can be administered to any and potentially all patients with a particular disorder. Second, administering particles of the present disclosure does not create an adaptive immune response to the delivered therapeutic like that typically raised against viral vector-based delivery systems and therefore embodiments can be re-dosed as needed for clinical effect. Administration of one or more ceDNA vectors in accordance with the present disclosure, such as in vivo delivery, is repeatable and robust.

In some embodiments, a portion or region of the GSH in a ceDNA vector as disclosed herein can be modified, e.g., where a point mutation can disrupt or knock-out the gene function of the GSH gene identified herein. In other embodiments, the portion or region of the GSH in a ceDNA vector can be modified to comprise a guide RNA (gRNA) inserted, e.g., a guide RNA for a nuclease as disclosed herein. In some embodiments, a ceDNA GSH vector can comprise a target site for a guide RNA (gRNA) as disclosed herein, or alternatively, a restriction cloning site for introduction of a nucleic acid of interest as disclosed herein. In another embodiment, a recombinase recognition site such as loxP may be introduced to facilitate directed recombination using a Cre recombinase expressed from rAAV or other gene transfer vector. The loxP site inserted into the GSH may also be used by breeding with transgenic mice that express Cre in a tissue specific manner.

In some embodiments, a ceDNA vector as disclosed herein can comprise recombinase recognition sites (RRS), for example, LoxP sites, attP, AttB sites and the like.

In some embodiments, a ceDNA vector useful in the methods and compositions as disclosed herein comprises a GSH nucleic acid sequence is between 30-1000 nucleotides, between 1-3 kb, between 3-5 kb, between 5-10 kb, or between 10-50 kb, between 50-100 kb, or between 100-300 kb or between 100-350 kb in size, or any integer between 30 base pairs and 350 kb.

(i) GSH and Homology Arms to GSH

In some embodiments, a ceDNA vector useful in the methods and compositions comprises a nucleic acid sequence comprising a first nucleic acid sequence comprising a 5′ region of the GSH, and a second nucleic sequence comprising a 3′ region of the GSH. In some embodiments, the 5′ region is within close proximity and upstream of a target site of integration and the 3′ region of the GSH is in close proximity and downstream of a target site of integration.

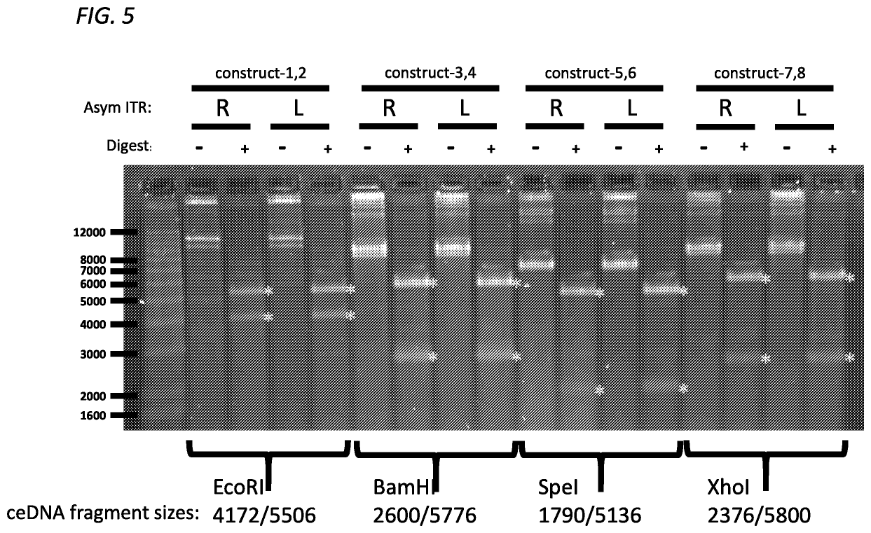

In some embodiments, a ceDNA vector useful in the methods and compositions comprises at least a portion of the PAX5 human genomic DNA or a fragment thereof, wherein the PAX5 is located at Chromosome 9: 36,833,275-37,034,185 reverse strand (GRCh38.p7:CM000671.2) or 36,833,272-37,034,182 in GRCh37 coordinates (see FIG. 5). In some embodiments, a ceDNA vector useful in the methods and compositions described herein comprises a nucleic acid sequence corresponding to at least a portion of untranslated a sequence or an intron of the PAX5 gene. In some embodiments, the untranslated sequence is a 5′UTR or 3′UTR or an intronic sequence of the PAX5 gene.

In some embodiments, a ceDNA vector useful in the methods and compositions comprises at least a portion of the Kif6 human genomic DNA or a fragment thereof, wherein the KIF6 is located at Chromosome 6: 39,329,990-39,725,405. In some embodiments, a ceDNA vector useful in the methods and compositions described herein comprises a nucleic acid sequence corresponding to at least a portion of untranslated a sequence or an intron of the KIF6 gene. In some embodiments, the untranslated sequence is a 5′UTR or 3′UTR or intronic sequence of the KIF6 gene.

In some embodiments, a ceDNA vector useful in the methods and compositions described herein comprises the genomic nucleic acid sequence, or a portion thereof, of any of the genes listed in Table 1A and Table 1B, herein. In some embodiments, the homology arms, e.g., HA-L and/or HA-R are each between about 200-800 nucleotides, e.g., about at least 200, or at least 300, or at least 400, or at least 500 or at least 600, or at least 700, or at least 800, or at least 900, or at least 1000, or at least 1100 or more than 1100 nucleotides in length.

TABLE 1A
candidate GSH regions or genes identified
using the methods disclosed herein.

| | Chromosomal | Accession number/ |
|---|---|---|
| Gene | location | location |
| PAX5 | Chromosome 9: | NC_000009.12 |
| | 36,833,275-37,034,185 | (36833274..37035949, |
| | reverse strand | complement) |
| MIR4540 | | NC_000009.12 |
| | | (36864254..36864308, |
| | | complement) |
| MIR4475 | GRCh38.p7 | NC_000009.12 |
| | (GCF_000001405.33) | (36823539..36823599, |
| | | complement) |
| MIR4476 | GRCh38.p7 | NC_000009.12 |
| | (GCF_000001405.33) | (36893462..36893531, |
| | | complement) |
| PRL32P21 | GRCh38.p7 | NC_000009.12 |
| | (GCF_000001405.33) | (37046835..37047242) |
| LOC105376031 | GRCh38.p7 | NC_000009.12 |
| | (GCF_000001405.33) | (37027763..37031333) |
| L0C105376032 | GRCh38.p7 | NC_000009.12 |
| | (GCF_000001405.33) | (37002697..37007774) |
| L0C105376030 | GRCh38.p7 | NC_000009.12 |
| | (GCF_000001405.33) | (36779475..36830456) |
| MELK | GRCh38.p7 | NC_000009.12 |
| | (GCF_000001405.33) | (36572862..36677683) |
| EBLN3P | GRCh38.p7 | NC_000009.12 |
| | (GCF_000001405.33) | (37079896..37090401) |
| ZCCHC7 | GRCh38.p7 | NC_000009.12 |
| | (GCF_000001405.33) | (37120169..37358149) |
| RNF38 | GRCh38.p7 | NC_000009.12 |
| | (GCF_000001405.33) | (36336398..36487384, |
| | | complement) |

TABLE 1B
intergenic loci and intragenic loci of candidate GSH regions
or genes identified using the methods disclosed herein
Intergenic Loci

| Taxonomic | Brief | | |
|---|---|---|---|
| Rank | description | Species | Chromosomal location |
| Macropodidae | mAAV_eye | M. | chromosome 1: |
| (taxonomic | integration | domestica | cdh 8: 674,639,xxx- |
| rank: | between | | 675,163,xxx |
| Family) | cadherin (cdh) | | cdh 10: 680,370,7xx- |
| | 8 and cdh | | 680,581, xxx |
| | 16. Because | | Intergenic |
| | the macropod | | distance = 5.2Mb |
| | genome is | | Empty EVE locus |
| | poorly | | in M. domestica |
| | annotated, | | 674,422,470- |
| | another | | 675,422,729 |
| | marsupial | Mouse | ch 9: |
| | Mondelphis | | cdh 8: 99,028,769- |
| | domesitca | | 99,416, 471 |
| | with a more | | cdh 11: 192,632,095- |
| | completely | | 102,785,111 |
| | assemble | | Intergenic distance = |
| | genome | | 3.2Mb |
| | is used as a | Homo | Chromosome 16 |
| | substitute | sapiens | cdh 8: 61,647,242- |
| | genome. | | 62,036,835 |
| | | | cdh 11: 64,943,753- |
| | | | 65,122,198 |
| | | | Intergenic distance = |
| | | | 2.9Mb |
| Leporidae | Leporidae EVE | H. | Chromosome 7: |
| (Family)- | located between | Sapiens | --KLH7->-- |
| the Family | NupL2 and | | NUPL2→GPNMB |
| Leporidae | GPNMB The | M. mus | --KLHL7->--NUPL2→ |
| are rabbits | gene order | | mir684—KCNH2 |
| and hares | is: <-Fam126A- - | | |
| species of the | KLH7-> | | |
| Lagomorph | NUPL2->--- | | |
| Order. | EVE------- | | |
| | GPNMB->--< | | |
| | IGF28P3— | | |
| | MALSU1 | | |

Intragenic loci

| | | | |
|---|---|---|---|
| Cetacea (Order) | EVE integrated | H. | chromosome 9: |
| | into an intron | sapiens | (Pax5) 36,833,275- |
| | of PAX5 | | 37,034,185 |
| | | M. mus | Chromosome 4: |
| | | | (Pax5) 44,531,506- |
| | | | 44,710,440 |
| (Family - | Myotis EVE | H. | Chromosome 6 |
| Vespertilionidae, | integrated into | sapiens | (Kif6) 39,329,990- |
| Order- | the Kif6 | | 39,725,405 |
| Chiroptera). | gene, intronic | | |
| Myotis (Genus), | | M. mus | Chromosome 17 |
| Myotinae | | | (Kif6) 49,754,497- |
| (Subfamily) | | | 50,049,172 |

B. ceDNA Vectors Comprising GSH Homology Arms (HA) for Integration of a Transgene at a GSH Locus

In alternative embodiments, the disclosure herein also relates to ceDNA vector composition comprising at least one GSH homology arm, e.g., a 5′ GSH homology arm (e.g., a HA-L), and/or a 3′GSH homology arm (e.g., a HA-R). In some embodiments, where the ceDNA vector comprises a 5′ GSH HA and a 3′ GSH HA, they flank a nucleic acid comprising a restriction cloning site, where the ceDNA vector can be used to integrate the flanked nucleic acid into the genome of the host's cell at a GSH by homologous recombination.

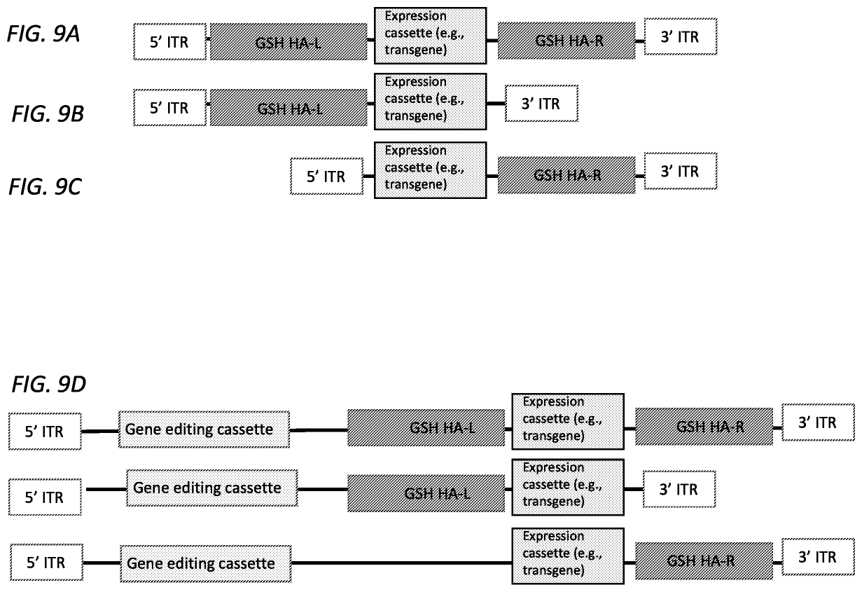

In some embodiments, a ceDNA vector as described herein are capsid-free, linear duplex DNA molecules formed from a continuous strand of complementary DNA with covalently-closed ends (linear, continuous and non-encapsulated structure), which comprises at least one ITR, or alternatively, two inverted terminal repeat (ITR) sequences, and where there are two ITRs, the two ITRs flank a nucleic acid construct, the nucleic acid construct comprising at least one homology arm, e.g., a left homology arm (also referred to as a HA-L or 5′ HA), a heterologous nucleic acid construct comprising at least one gene of interest (GOI) (or transgene), and/or a right homology arm (also referred to as a HA-R or 3′HA). FIGS. 9A-9C show exemplary ceDNA vector constructs comprising the transgene for insertion into a GSH locus, flanked by either a 5′ GSH HA and a 3′ GSH HA (FIG. 9A), or a transgene linked to a 5′ GSH HA (FIG. 9B), or a transgene linked to a 3′ GSH-HA (FIG. 9C). In some embodiments, the GOI can be genomic DNA (gDNA) encoding a protein or nucleic acid of interest, where the GOI has an open reading frame (ORF) and comprises introns and exons, or alternatively, the GOI can be complementary DNA (cDNA) i.e., lacking introns). In some embodiments, the GOI can be operatively linked to any one or more of: a promoter or regulatory switch as defined herein, a 5′ UTR, a 3′ UTR, a polyadenylation sequence, post-transcriptional elements which is operatively linked to a promoter or other regulatory switch as described herein. An exemplary ceDNA vector for insertion of a GOI into a GSH as described herein is shown in FIG. 1A. The 5′ ITR and the 3′ ITR of a ceDNA vector as disclosed herein can have the same symmetrical three-dimensional organization with respect to each other, (i.e., symmetrical or substantially symmetrical), or alternatively, the 5′ ITR and the 3′ ITR can have different three-dimensional organization with respect to each other (i.e., asymmetrical ITRs), as these terms are defined herein. In addition, the ITRs can be from the same or different serotypes. In some embodiments, a ceDNA vector can comprise ITR sequences that have a symmetrical three-dimensional spatial organization such that their structure is the same shape in geometrical space, or have the same A, C-C′ and B-B′ loops in 3D space (i.e., they are the same or are mirror images with respect to each other). In some embodiments, one ITR can be from one AAV serotype, and the other ITR can be from a different AAV serotype.

Accordingly, one aspect of the technology described herein relates to a close-ended DNA (ceDNA) vector composition comprising at least one ITR, or two ITRs flanking, in the following order; (σ) a GSH 5′ homology arm (also referred to herein as “HA-L”, “5′ GSH-specific homology arm” or “5′ GSH-HA”), (b) a nucleic acid sequence comprising a restriction cloning site, and (c) a GSH 3′ homology arm (also referred to herein as “HA-R”, “3′ GSH-specific homology arm” or “3′ GSH-HA”), where the 5′ homology arm (HA-L) and the 3′ homology arm (HA-R) bind to a target site located in a genomic safe harbor locus identified according to the methods as disclosed herein, and wherein the 5′ and 3′ homology arms allow insertion (of the nucleic acid located between the homology arms) by homologous recombination into a locus located within the genomic safe. In some embodiments, the ceDNA is a linear closed ended duplex DNA.

In some embodiments, a ceDNA vector described herein for integration of a nucleic acid of interest into a GSH locus can comprise: a first ITR, a 5′ GSH specific HA (HA-L), a nucleic acid of interest and/or an expressible transgene cassette (e.g., a sequence that encodes a therapeutic protein or nucleic acid as described herein, and/or a reporter protein), and/or a 3′GSH HA (HA-R), and a second ITR. For example, in some embodiments, a ceDNA vector can comprise: a first ITR, a 5′ GSH specific HA (HA-L), a nucleic acid of interest and/or an expressible transgene cassette (e.g., a sequence that encodes a therapeutic protein or nucleic acid as described herein, and/or a reporter protein), and a 3′GSH HA (HA-R), and a second ITR. In alternative embodiments, a ceDNA vector can comprise: a first ITR, a 5′ GSH specific HA (HA-L), a nucleic acid of interest and/or an expressible transgene cassette (e.g., a sequence that encodes a therapeutic protein or nucleic acid as described herein, and/or a reporter protein), and a second ITR. In alternative embodiments, a ceDNA vector can comprise: a first ITR, a nucleic acid of interest and/or an expressible transgene cassette (e.g., a sequence that encodes a therapeutic protein or nucleic acid as described herein, and/or a reporter protein), and a 3′GSH HA (HA-R), and a second ITR. In some embodiments, such ceDNA vectors comprise a first ITR only (e.g., a 5′ ITR but do not comprise a 3′ ITR). In alternative embodiments, such ceDNA vectors can comprise a second ITR only (e.g., a 3′ ITR) and not a 5′ ITR. In some embodiments, such ceDNA vectors can also comprise a gene editing cassette as described herein, e.g., located 3′ of the 5′ ITR (first ITR), but 5′ of the 5′ homology arm. In alternative embodiments, a ceDNA vector can also comprise a gene editing cassette as described herein, e.g, located 5′ of the 3′ ITR (second ITR), but 3′ of the 3′ homology arm. In some embodiments, where the gene editing cassette comprises a guide RNA (gRNA) or guide DNA (gDNA), the gDNA or gRNA targets a region in the 5′ GSH-HA and/or in the 3′ GSH-HA.

In some embodiments, a ceDNA vector described herein for integration of a nucleic acid of interest into a GSH locus can comprise: a first ITR, a guide RNA (gRNA) or guide DNA (gDNA) which targets a region in the GSH locus, a nucleic acid of interest and/or an expressible transgene cassette (e.g., a sequence that encodes a therapeutic protein or nucleic acid as described herein, and/or a reporter protein), and a second ITR.

In some embodiments the TRs are inverted ITRs (ITRs). In some embodiments, one of the ITRs is a wild-type or modified AAV ITR. In some embodiments, the ITRS are not AAV ITRs. The ceDNA vectors can comprise e.g., one or more gene editing molecules, as described in International Patent Application PCT/US18/064242, filed on Dec. 6, 2018, which is specifically incorporated herein in its entirety by reference. The ceDNA vectors have the advantage of being able to comprise all of the components of the gene editing system.

In some embodiments, a ceDNA vector described herein for integration of a nucleic acid of interest into a GSH locus can comprise in this order: a) a first TR, e.g., ITR, b) a 5′ GSH-specific homology arm, c) a restriction cloning site, d) a 3′ GSH-specific homology arm, and e) a second TR, e.g., ITR. In some embodiments, the ITRs can be asymmetric or symmetric or substantially symmetric with respect to each other, as disclosed herein.

As described above, a ceDNA vector for insertion of a transgene at a GSH locus as disclosed herein, comprises any one of: an asymmetrical ITR pair, a symmetrical ITR pair, or substantially symmetrical ITR pair as described above, that flank a HA-L and HA-R, and located between the HA-L and HA-R is a transgene (or donor sequence) to be inserted into the genome of a host cell at a GSH locus disclosed in Tables 1A or 1B. FIG. 1A shows an exemplary ceDNA vector for insertion of a transgene into the genome of a host cells at a specific GSH locus. FIGS. 1B-1H show schematics of embodiments of FIG. 1A showing functional components of a ceDNA vector of the present disclosure. In other embodiments, a ceDNA vector can comprise one GSH homology arm, e.g., see FIG. 9B and FIG. 9C, where the ceDNA vector comprises a 5′ GSH-HA (HA-L) or a 3′ GSH-HA (HA-R). ceDNA vectors are capsid-free and can be obtained from a plasmid encoding in this order: a first ITR, a HA-L, an expressible transgene cassette, HA-R, and a second ITR, where the first and second ITR sequences are asymmetrical, symmetrical or substantially symmetrical relative to each other as defined herein. ceDNA vectors are capsid-free and can be obtained from a plasmid encoding in this order: a first ITR, a HA-L, an expressible transgene (protein or nucleic acid), a HA-R and a second ITR, where the first and second ITR sequences are asymmetrical, symmetrical or substantially symmetrical relative to each other as defined herein. In some embodiments, the expressible transgene cassette includes, as needed: an enhancer/promoter, one or more homology arms, a donor sequence, a post-transcription regulatory element (e.g., WPRE, e.g., SEQ ID NO: 67)), and a polyadenylation and termination signal (e.g., BGH polyA, e.g., SEQ ID NO: 68).

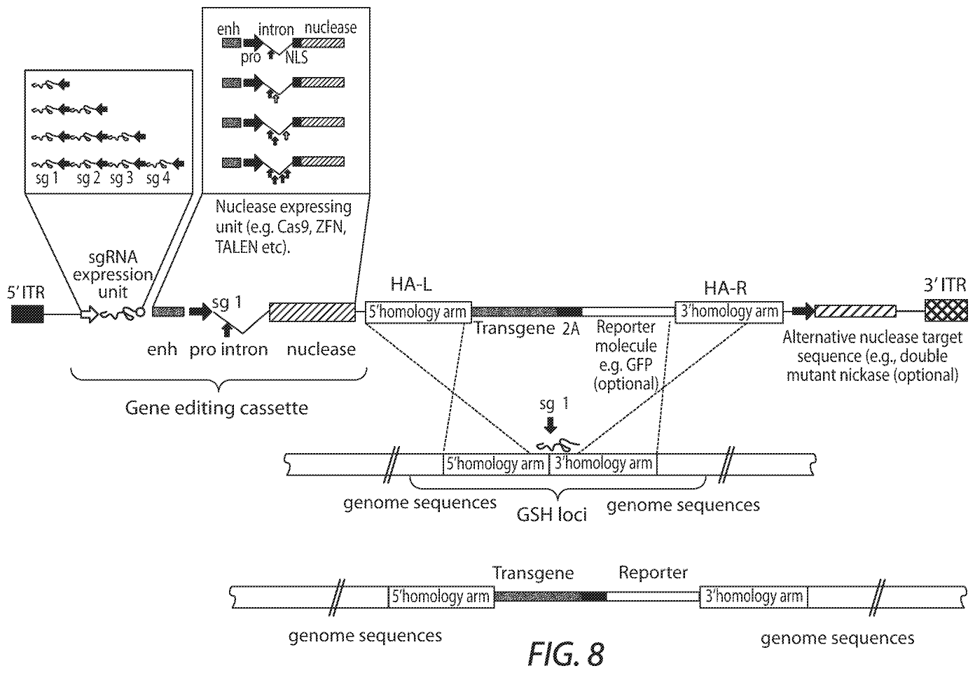

In alternative embodiments, in addition to a ceDNA vector comprising ITRs flanking a HA-L and HA-R, which in turn flank the transgene to be inserted, the ceDNA vector can further include a “gene editing cassette” located between the ITRs, but outside the homology arms. Exemplary “all-in-one” ceDNA vector for insertion of a gene into a GSH locus are shown in FIGS. 8, 9D and 10. Such all-in one ceDNA vectors for insertion of a transgene into a GSH locus can comprise at least one of the following: a nuclease, a guide RNA, an activator RNA, and a control element. Accordingly, in certain embodiments, a ceDNA vector comprises two ITRs, a gene editing cassette comprising at least two components of a gene editing system, (e.g. a nuclease such as CAS and at least one gRNA, or two ZNFs, etc.), and a transgene flanked by a HA-L and HA-R that are specific to a GSH locus shown in Table 1A or 1B, Thus, in some embodiments, the ceDNA vectors comprise two ITRs, a transgene flanked by HA-L and HA-R, and multiple components of a gene editing system, including a gene editing molecule of interest (e.g., a nuclease (e.g., sequence specific nuclease), one or more guide RNA, Cas or other ribonucleoprotein (RNP), or any combination thereof. In some embodiments, a nuclease can be inactivated/diminished after gene editing, reducing or eliminating off-target editing, if any, that would otherwise occur with the persistence of an added nuclease within cells.

In some embodiments, even if viral ITRs are used, a ceDNA vector as described herein is a non-viral, capsid-free vector, i.e. there is no physical contact with the viral capsid protein from which the ITR is derived.

In embodiments, the ceDNA vector of the present disclosure may include an inverted terminal repeat (e.g. ITR) structure that is mutated or altered with respect to the wild type TR structure disclosed herein, but still retains an operable RBE, (e.g. Rep binding element), terminal resolution site, and RBE′ portion. In embodiments, the ceDNA vector of the present disclosure may include an ITR structure that is mutated or altered with respect to the wild type AAV2 ITR structure disclosed herein, but still retains an operable RBE, trs and RBE′ portion.

In some embodiments, the 3′ and 5′ homology arms complementary base pair with regions of the GSH identified according to the methods as disclosed herein. In some embodiments, 3′ and 5′ homology arms (HA) flank a target site of integration, e.g., target insertion loci in the GSH as disclosed herein. In some embodiments, the 3′ homology arm complementary base pairs with a nucleic acid region 3′ (i.e., upstream) of a target site of integration or target insertion loci of the GSH, and 5′ homology arm complementary base pairs with a nucleic acid region 5′ (i.e., downstream) of a target site of integration or target insertion locus of the GSH. In some embodiments, the 5′ and 3′ homology arms are complementary to, e.g., at least 60%, or at least 70%, or at least 80%, or at least 85%, or at least 90%, or at least 91%, or at least 92%, or at least 93%, or at least 94%, or at least 94%, or at least 96%, or at least 97%, or at least 98%, or at least 99%, or at least 99.5% complementary to portions of nucleic acid regions identified as a GSH herein.

For integration of the nucleic acid located between the 5′ and 3′ homology arms of the ceDNA vector, the 5′ and 3′ homology arms should be long enough for targeting to the GSH and allow (e.g., guide) integration into the genome by homologous recombination. For example, the ceDNA vector may contain nucleotides encoding 5′ and 3′ homology arms for directing integration by homologous recombination into the genome of the host cell at a precise location(s) in the GSH identified herein.

To increase the likelihood of integration at a precise location, the 5′ and 3′ homology arms may include a sufficient number of nucleic acids, such as 50 to 5,000 base pairs, or 100 to 5,000 base pairs, or 500 to 5,000 base pairs, which have a high degree of sequence identity or homology to the corresponding target sequence to enhance the probability of homologous recombination. The 5′ and 3′ homology arms may be any sequence that is homologous with the GSH target sequence in the genome of the host cell. That is, the 5′ and 3′ homology arms are complementary to portions of the GSH target sequence identified herein. Furthermore, the 5′ and 3′ homology arms may be non-encoding or encoding nucleotide sequences. In some embodiments, the homology between the 5′ homology arm and the corresponding sequence on the chromosome is at least any of 80%, 85%, 90%, 95%, 97%, 98%, 99%, or 100%. In embodiments, the homology between the 3′ homology arm and the corresponding sequence on the chromosome is at least any of 80%, 85%, 90%, 95%, 97%, 98%, 99%, or 100%. In embodiments, the 5′ and/or 3′ homology arms can be homologous to a sequence immediately upstream and/or downstream of the integration or DNA cleavage site on the chromosome. Alternatively, the 5′ and/or 3′ homology arms can be homologous to a sequence that is distant from the integration or DNA cleavage site, such as at least 1, 2, 5, 10, 15, 20, 25, 30, 50, 100, 200, 300, 400, or 500 bp away from the integration or DNA cleavage site, or partially or completely overlapping with the DNA cleavage site. In embodiments, the 3′ homology arm of the nucleotide sequence is proximal to the altered ITR.

In some embodiments, the 5′ and/or 3′ homology arm can be any length, e.g., between 30-2000 bp. In some embodiments, the 5′ and/or 3′ homology arms are between 200-350 bp long. Details study regarding length of homology arms and recombination frequency is e.g., reported by Zhang et al. “Efficient precise knockin with a double cut HDR donor after CRISPR/Cas9-mediated double-stranded DNA cleavage.” Genome biology 18.1 (2017): 35, which is incorporated herein in its entity by reference.

In some embodiments, the GSH 5′ homology arm and the GSH 3′ homology arm bind to target sites that are spatially distinct nucleic acid sequences in the genomic safe harbor identified according to the methods as disclosed herein.

In some embodiments, a ceDNA vector composition for integration of a nucleic acid of interest into a GSH locus can comprises a 5′ GSH-specific homology arm and the GSH 3′ GSH-specific homology arm that are at least 65% complementary to a target sequence in the genomic safe harbor locus identified according to the methods disclosed herein. In some embodiments, the ceDNA vector as disclosed herein comprises a 5′ GSH-specific homology arm and the 3′ GSH-specific homology arm that bind to a target site located in the PAX5 genomic safe harbor sequence, or a gene listed in Table 1A or Table 1B herein. In one embodiment, a ceDNA vector composition as described herein for integration of a nucleic acid of interest into a GSH locus does not contain any prokaryotic DNA sequence elements, for example minicircle-DNA (mcDNA), but it is contemplated that some prokaryotic-sourced DNA may be inserted as an exogenous sequence.

In embodiments, the ceDNA vector of the present disclosure may include a terminal repeat (e.g. ITR) structure that is mutated or altered with respect to the wild type TR structure disclosed herein, but still retains an operable rolling circle binding element (RBE), terminal resolution site, and RBE′ portion. In embodiments, the ceDNA vector of the present disclosure may include an ITR structure that is mutated or altered with respect to the wild type AAV2 ITR structure disclosed herein, but still retains an operable RBE, trs and RBE′ portion. In some embodiments, an RBE is not used, but a different rolling circle binding element.

In embodiments, the ceDNA vector of the present disclosure may include an engineered ITR structure comprising a rolling circle replication origin.

C. ceDNA Vectors Comprising a Gene Editing Transgene

An exemplary ceDNA vectors with a 5′ GSH-specific homology arm and a 3′ GSH-specific homology arm are made where the 5′ GSH-specific homology arm and a 3′ GSH-specific homology arm are specific to a GSH identified herein, e.g., Pax5 or a GSH identified in Table 1A or Table 1B. Accordingly, in some embodiments, a ceDNA vector can comprise in this order: a first ITR, a 5′ GSH-specific homology arm (i.e., a HA-L), an expression cassette (e.g., a transgene or other GOI, which can be operatively linked to a regulatory switch, promoters, polyA, enhancers, and can also comprise 5′ UTR and 3′ UTR sequences where the GOI is gDNA), a 3′ GSH-specific homology arm (a HA-R), and a second ITR), where the first and second ITRs can be symmetrical, substantially symmetrical or asymmetrical relative to each other, as defined herein. In some embodiments, the ceDNA vector may further comprise between the ITRs, a gene editing molecule, e.g. one or more of, at least one guide RNA directed to the GSH, and a nuclease (e.g., Cas9) CRISPR/Cas, ZFN or Tale nucleic acid sequences.

A ceDNA vector for insertion of a transgene at a GSH as described herein comprises a transgene to be inserted (also referred to herein as a donor sequence) that is flanked by GSH-specific 5′ and 3′ homology arms, can further include a gene editing cassette outside of the Homology arm region. A gene editing cassette can comprise one or more gene editing molecules as described in International Application PCT/US2018/064242, filed on Dec. 6, 2018, which is incorporated herein in its entirety by reference. For example, a ceDNA vector encompassed in the methods and compositions as disclosed herein may include one or more of: a 5′ homology arm, a 3′ homology arm, a polyadenylation site upstream and proximate to the 5′ homology arm, where the HA-L and HA-R target the Pax5 gene, or a GSH identified in Table 1A or Table 1B, and where the ceDNA vector also encodes a gene editing molecule, e.g. one or more of, at least one guide RNA directed to the GSH, and a nuclease (e.g., Cas9) CRISPR/Cas, ZFN or Tale nucleic acid sequences

D. ceDNA Vectors in General

The ceDNA vectors for insertion of a GOI or transgene into a GSH as described herein are not limited by size, thereby permitting, for example, expression of all of the components necessary for both the insertion of the transgene or GOI into the GSH, as well as expression of a transgene from a the GSH locus in the host's genome. The ceDNA vector is preferably duplex, e.g. self-complementary, over at least a portion of the molecule, such as the expression cassette (e.g. ceDNA is not a double stranded circular molecule). The ceDNA vector has covalently closed ends, and thus is resistant to exonuclease digestion (e.g. exonuclease I or exonuclease III), e.g. for over an hour at 37° C. In some embodiments, a ceDNA vector as disclosed herein is translocated to the nucleus where expression of the transgene in the ceDNA vector, e.g., genetic medicine transgene can occur. In some embodiments, a ceDNA vector as disclosed herein translocated to the nucleus where expression of the transgene, e.g., genetic medicine transgene located between the two ITRs can occur.

In general, a ceDNA vector disclosed herein useful for insertion of a transgene into a GSH of a hosts genome, comprises in the 5′ to 3′ direction: a first adeno-associated virus (AAV) inverted terminal repeat (ITR), a HA-L, a nucleotide sequence of interest (for example an expression cassette as described herein), a HA-R, and a second AAV ITR. The ITR sequences selected from any of: (i) at least one WT ITR and at least one modified AAV inverted terminal repeat (mod-ITR) (e.g., asymmetric modified ITRs); (ii) two modified ITRs where the mod-ITR pair have a different three-dimensional spatial organization with respect to each other (e.g., asymmetric modified ITRs), or (iii) symmetrical or substantially symmetrical WT-WT ITR pair, where each WT-ITR has the same three-dimensional spatial organization, or (iv) symmetrical or substantially symmetrical modified ITR pair, where each mod-ITR has the same three-dimensional spatial organization.

An exemplary ceDNA vector useful for insertion of a GOI or transgene into a GSH comprises two inverted terminal repeat (ITR) sequences flanking a nucleic acid construct, the nucleic acid construct comprising a left homology arm (also referred to as a HA-L or 5′ HA), a heterologous nucleic acid construct comprising at least one gene of interest (GOI) (or transgene), and a right homology arm (also referred to as a HA-R or 3′HA). In some embodiments, the GOI can be operatively linked to any one or more of: a promoter or regulatory switch as defined herein, a 5′ UTR, a 3′ UTR, a polyadenylation sequence, post-transcriptional elements which is operatively linked to a promoter or other regulatory switch as described herein.

An exemplary ceDNA vector for insertion of a GOI into a GSH as described herein is shown in FIG. 1A. Additionally, FIGS. 1B-1G show schematics of nonlimiting, exemplary ceDNA vectors, or the corresponding sequence of ceDNA plasmids. These show an embodiment with two ITRs flanking the 5′ GSH HA and a 3′ GSH HA, however, it is envisioned that only one ITR can be used, and/or one GSH homology arm (e.g., a 5′ GSH HA or a 3′ GSH HA) can be used, e.g., see FIGS. 9B, 9C. ceDNA vectors are capsid-free and can be obtained from a plasmid encoding in this order: a first ITR, an expression cassette comprising a transgene and a second ITR. The expression cassette may include one or more regulatory sequences that allows and/or controls the expression of the transgene, e.g., where the expression cassette can comprise one or more of, in this order: an enhancer/promoter, an ORF reporter (transgene), a post-transcription regulatory element (e.g., WPRE), and a polyadenylation and termination signal (e.g., BGH polyA).

The expression cassette can also comprise an internal ribosome entry site (IRES) (e.g., SEQ ID NO: 190) and/or a 2A element. The cis-regulatory elements include, but are not limited to, a promoter, a riboswitch, an insulator, a mir-regulatable element, a post-transcriptional regulatory element, a tissue- and cell type-specific promoter and an enhancer. In some embodiments the ITR can act as the promoter for the transgene. In some embodiments, the ceDNA vector comprises additional components to regulate expression of the transgene, for example, a regulatory switch, which are described herein in the section entitled “Regulatory Switches” for controlling and regulating the expression of the transgene, and can include if desired, a regulatory switch which is a kill switch to enable controlled cell death of a cell comprising a ceDNA vector.

The expression cassette can comprise more than 4000 nucleotides, 5000 nucleotides, 10,000 nucleotides or 20,000 nucleotides, or 30,000 nucleotides, or 40,000 nucleotides or 50,000 nucleotides, or any range between about 4000-10,000 nucleotides or 10,000-50,000 nucleotides, or more than 50,000 nucleotides. In some embodiments, the expression cassette can comprise a transgene in the range of 500 to 50,000 nucleotides in length. In some embodiments, the expression cassette can comprise a transgene in the range of 500 to 75,000 nucleotides in length. In some embodiments, the expression cassette can comprise a transgene which is in the range of 500 to 10,000 nucleotides in length. In some embodiments, the expression cassette can comprise a transgene which is in the range of 1000 to 10,000 nucleotides in length. In some embodiments, the expression cassette can comprise a transgene which is in the range of 500 to 5,000 nucleotides in length. The ceDNA vectors do not have the size limitations of encapsidated AAV vectors, thus enable delivery of a large-size expression cassette to provide efficient transgene. In some embodiments, the ceDNA vector is devoid of prokaryote-specific methylation.

ceDNA expression cassette can include, for example, an expressible exogenous sequence (e.g., open reading frame) or transgene that encodes a protein that is either absent, inactive, or insufficient activity in the recipient subject or a gene that encodes a protein having a desired biological or a therapeutic effect. The transgene can encode a gene product that can function to correct the expression of a defective gene or transcript. In principle, the expression cassette can include any gene that encodes a protein, polypeptide or RNA that is either reduced or absent due to a mutation or which conveys a therapeutic benefit when overexpressed is considered to be within the scope of the disclosure.

The expression cassette can comprise any transgene useful for treating a disease or disorder in a subject. A ceDNA vector can be used to deliver and express any gene of interest in the subject, which includes but are not limited to, nucleic acids encoding polypeptides, or non-coding nucleic acids (e.g., RNAi, miRs etc.), as well as exogenous genes and nucleotide sequences, including virus sequences in a subjects' genome, e.g., HIV virus sequences and the like. Preferably a ceDNA vector disclosed herein is used for therapeutic purposes (e.g., for medical, diagnostic, or veterinary uses) or immunogenic polypeptides. In certain embodiments, a ceDNA vector is useful to express any gene of interest in the subject, which includes one or more polypeptides, peptides, ribozymes, peptide nucleic acids, siRNAs, RNAis, antisense oligonucleotides, antisense polynucleotides, or RNAs (coding or non-coding; e.g., siRNAs, shRNAs, micro-RNAs, and their antisense counterparts (e.g., antagoMiR)), antibodies, antigen binding fragments, or any combination thereof.

The expression cassette can also encode polypeptides, sense or antisense oligonucleotides, or RNAs (coding or non-coding; e.g., siRNAs, shRNAs, micro-RNAs, and their antisense counterparts (e.g., antagoMiR)). Expression cassettes can include an exogenous sequence that encodes a reporter protein to be used for experimental or diagnostic purposes, such as β-lactamase, β-galactosidase (LacZ), alkaline phosphatase, thymidine kinase, green fluorescent protein (GFP), chloramphenicol acetyltransferase (CAT), luciferase, and others well known in the art.

Sequences provided in the expression cassette, expression construct of a ceDNA vector described herein can be codon optimized for the target host cell. As used herein, the term “codon optimized” or “codon optimization” refers to the process of modifying a nucleic acid sequence for enhanced expression in the cells of the vertebrate of interest, e.g., mouse or human, by replacing at least one, more than one, or a significant number of codons of the native sequence (e.g., a prokaryotic sequence) with codons that are more frequently or most frequently used in the genes of that vertebrate. Various species exhibit particular bias for certain codons of a particular amino acid. Typically, codon optimization does not alter the amino acid sequence of the original translated protein. Optimized codons can be determined using e.g., Aptagen's Gene Forge® codon optimization and custom gene synthesis platform (Aptagen, Inc., 2190 Fox Mill Rd. Suite 300, Herndon, Va. 20171) or another publicly available database.

In some embodiments, a transgene expressed by the ceDNA vector for insertion of a transgene at a GSH locus as disclosed herein is a therapeutic gene. In some embodiments, a therapeutic gene is an antibody, or antibody fragment, or antigen-binding fragment thereof, or a fusion protein. In some embodiments, the antibody or fusion protein thereof is an activating antibody or a neutralizing antibody or antibody fragment and the like. In some embodiments, a ceDNA vector for controlled gene expression comprises an antibody or fusion protein as disclosed in International patent PCT/US19/18016, filed on Feb. 14, 2019, which is incorporated herein in its entirety by reference.

In particular, a therapeutic gene is one or more therapeutic agent(s), including, but not limited to, for example, protein(s), polypeptide(s), peptide(s), enzyme(s), antibodies, antigen binding fragments, as well as variants, and/or active fragments thereof, for use in the treatment, prophylaxis, and/or amelioration of one or more symptoms of a disease, dysfunction, injury, and/or disorder. Exemplary therapeutic genes are described herein in the section entitled “Method of Treatment”.

There are many structural features of ceDNA vectors that differ from plasmid-based expression vectors. ceDNA vectors may possess one or more of the following features: the lack of original (i.e. not inserted) bacterial DNA, the lack of a prokaryotic origin of replication, being self-containing, i.e., they do not require any sequences other than the two ITRs, including the Rep binding and terminal resolution sites (RBS and TRS), and an exogenous sequence between the ITRs, the presence of ITR sequences that form hairpins, and the absence of bacterial-type DNA methylation or indeed any other methylation considered abnormal by a mammalian host. In general, it is preferred for the present vectors not to contain any prokaryotic DNA but it is contemplated that some prokaryotic DNA may be inserted as an exogenous sequence, as a nonlimiting example in a promoter or enhancer region. Another important feature distinguishing ceDNA vectors from plasmid expression vectors is that ceDNA vectors are single-strand linear DNA having closed ends, while plasmids are always double-strand DNA.

ceDNA vectors produced by the methods provided herein preferably have a linear and continuous structure rather than a non-continuous structure, as determined by restriction enzyme digestion assay (FIG. 4D). The linear and continuous structure is believed to be more stable from attack by cellular endonucleases, as well as less likely to be recombined and cause mutagenesis. Thus, a ceDNA vector in the linear and continuous structure is a preferred embodiment. The continuous, linear, single strand intramolecular duplex ceDNA vector can have covalently bound terminal ends, without sequences encoding AAV capsid proteins. These ceDNA vectors are structurally distinct from plasmids (including ceDNA plasmids described herein), which are circular duplex nucleic acid molecules of bacterial origin. The complimentary strands of plasmids may be separated following denaturation to produce two nucleic acid molecules, whereas in contrast, ceDNA vectors, while having complimentary strands, are a single DNA molecule and therefore even if denatured, remain a single molecule. In some embodiments, ceDNA vectors as described herein can be produced without DNA base methylation of prokaryotic type, unlike plasmids. Therefore, the ceDNA vectors and ceDNA-plasmids are different both in term of structure (in particular, linear versus circular) and also in view of the methods used for producing and purifying these different objects (see below), and also in view of their DNA methylation which is of prokaryotic type for ceDNA-plasmids and of eukaryotic type for the ceDNA vector.

There are several advantages of using a ceDNA vector as described herein over plasmid-based expression vectors, such advantages include, but are not limited to: 1) plasmids contain bacterial DNA sequences and are subjected to prokaryotic-specific methylation, e.g., 6-methyl adenosine and 5-methyl cytosine methylation, whereas capsid-free AAV vector sequences are of eukaryotic origin and do not undergo prokaryotic-specific methylation; as a result, capsid-free AAV vectors are less likely to induce inflammatory and immune responses compared to plasmids; 2) while plasmids require the presence of a resistance gene during the production process, ceDNA vectors do not; 3) while a circular plasmid is not delivered to the nucleus upon introduction into a cell and requires overloading to bypass degradation by cellular nucleases, ceDNA vectors contain viral cis-elements, i.e., ITRs, that confer resistance to nucleases and can be designed to be targeted and delivered to the nucleus. It is hypothesized that the minimal defining elements indispensable for ITR function are a Rep-binding site (RBS; 5′-GCGCGCTCGCTCGCTC-3′ (SEQ ID NO: 60) for AAV2) and a terminal resolution site (TRS; 5′-AGTTGG-3′ (SEQ ID NO: 64) for AAV2) plus a variable palindromic sequence allowing for hairpin formation; and 4) ceDNA vectors do not have the over-representation of CpG dinucleotides often found in prokaryote-derived plasmids that reportedly binds a member of the Toll-like family of receptors, eliciting a T cell-mediated immune response. In contrast, transductions with capsid-free AAV vectors disclosed herein can efficiently target cell and tissue-types that are difficult to transduce with conventional AAV virions using various delivery reagent.

Encompassed herein are methods and compositions comprising a ceDNA vector for insertion of a GOI or transgene into a GSH as described herein, which may further include a delivery system, such as but not limited to, a liposome nanoparticle delivery system. Nonlimiting exemplary liposome nanoparticle systems encompassed for use are disclosed herein. In some aspects, the disclosure provides for a lipid nanoparticle comprising ceDNA and an ionizable lipid. For example, a lipid nanoparticle formulation that is made and loaded with a ceDNA vector obtained by the process is disclosed in International Application PCT/US2018/050042, filed on Sep. 7, 2018, which is incorporated herein.

The ceDNA vectors as disclosed herein have no packaging constraints imposed by the limiting space within the viral capsid. ceDNA vectors represent a viable eukaryotically-produced alternative to prokaryote-produced plasmid DNA vectors, as opposed to encapsulated AAV genomes. This permits the insertion of control elements, e.g., regulatory switches as disclosed herein, large transgenes, multiple transgenes etc.

### IV. ITRs

As disclosed herein, ceDNA vectors useful for insertion of a transgene into a GSH of a subject's genome contain a transgene or heterologous nucleic acid sequence positioned between a HA-L and a HA-R, which in turn is flanked by two inverted terminal repeat (ITR) sequences, where the ITR sequences can be an asymmetrical ITR pair or a symmetrical- or substantially symmetrical ITR pair, as these terms are defined herein. A ceDNA vector as disclosed herein can comprise ITR sequences that are selected from any of: (i) at least one WT ITR and at least one modified AAV inverted terminal repeat (mod-ITR) (e.g., asymmetric modified ITRs); (ii) two modified ITRs where the mod-ITR pair have a different three-dimensional spatial organization with respect to each other (e.g., asymmetric modified ITRs), or (iii) symmetrical or substantially symmetrical WT-WT ITR pair, where each WT-ITR has the same three-dimensional spatial organization, or (iv) symmetrical or substantially symmetrical modified ITR pair, where each mod-ITR has the same three-dimensional spatial organization, where the methods of the present disclosure may further include a delivery system, such as but not limited to a liposome nanoparticle delivery system.

In some embodiments, the ITR sequence can be from viruses of the Parvoviridae family, which includes two subfamilies: Parvovirinae, which infect vertebrates, and Densovirinae, which infect insects. The subfamily Parvovirinae (referred to as the parvoviruses) includes the genus Dependovirus, the members of which, under most conditions, require coinfection with a helper virus such as adenovirus or herpes virus for productive infection. The genus Dependovirus includes adeno-associated virus (AAV), which normally infects humans (e.g., serotypes 2, 3A, 3B, 5, and 6) or primates (e.g., serotypes 1 and 4), and related viruses that infect other warm-blooded animals (e.g., bovine, canine, equine, and ovine adeno-associated viruses). The parvoviruses and other members of the Parvoviridae family are generally described in Kenneth I. Berns, “Parvoviridae: The Viruses and Their Replication,” Chapter 69 in FIELDS VIROLOGY (3d Ed. 1996).

While ITRs exemplified in the specification and Examples herein are AAV2 WT-ITRs, one of ordinary skill in the art is aware that one can as stated above use ITRs from any known parvovirus, for example a dependovirus such as AAV (e.g., AAV1, AAV2, AAV3, AAV4, AAV5, AAV 5, AAV7, AAV8, AAV9, AAV10, AAV 11, AAV12, AAVrh8, AAVrh10, AAV-DJ, and AAV-DJ8 genome. E.g., NCBI: NC 002077; NC 001401; NC001729; NC001829; NC006152; NC 006260; NC 006261), chimeric ITRs, or ITRs from any synthetic AAV. In some embodiments, the AAV can infect warm-blooded animals, e.g., avian (AAAV), bovine (BAAV), canine, equine, and ovine adeno-associated viruses. In some embodiments the ITR is from B19 parvovirus (GenBank Accession No: NC 000883), Minute Virus from Mouse (MVM) (GenBank Accession No. NC 001510); goose parvovirus (GenBank Accession No. NC 001701); snake parvovirus 1 (GenBank Accession No. NC 006148). In some embodiments, the 5′ WT-ITR can be from one serotype and the 3′ WT-ITR from a different serotype, as discussed herein.

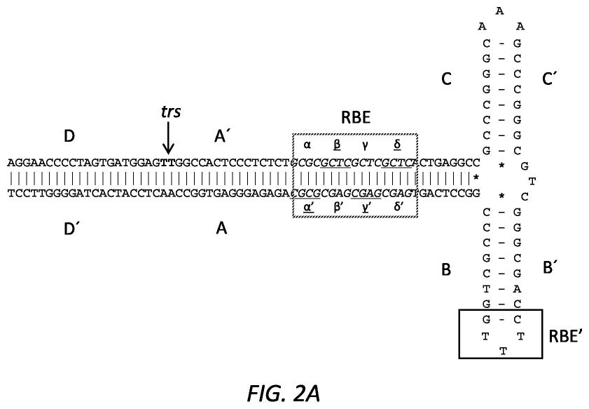

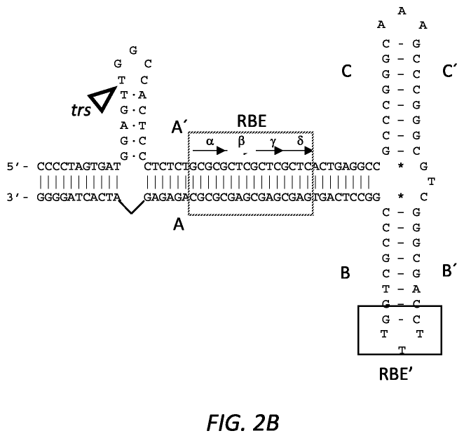

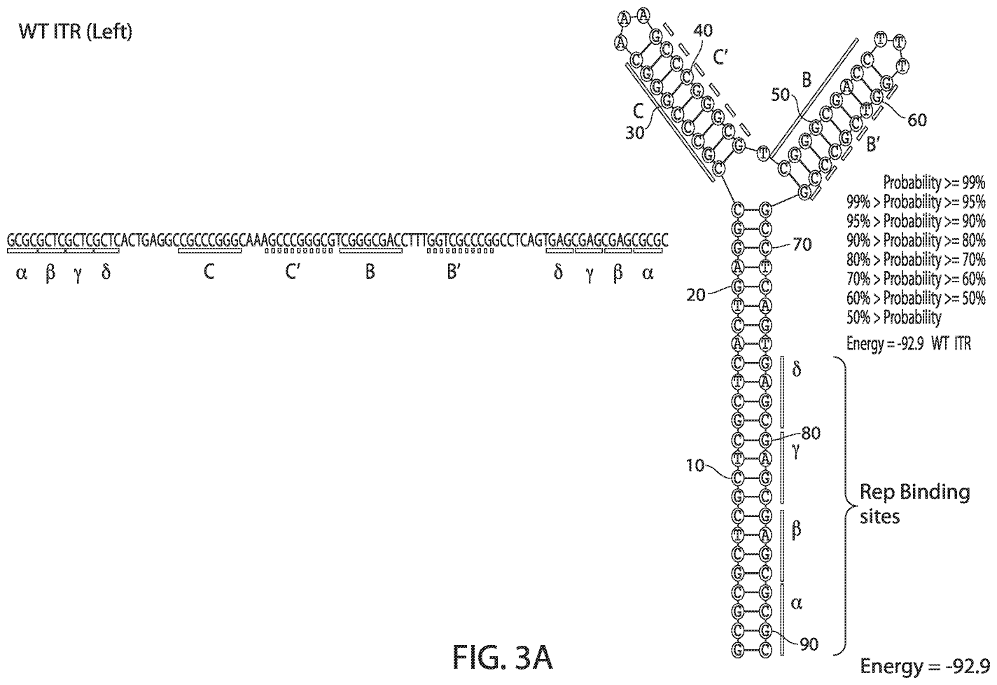

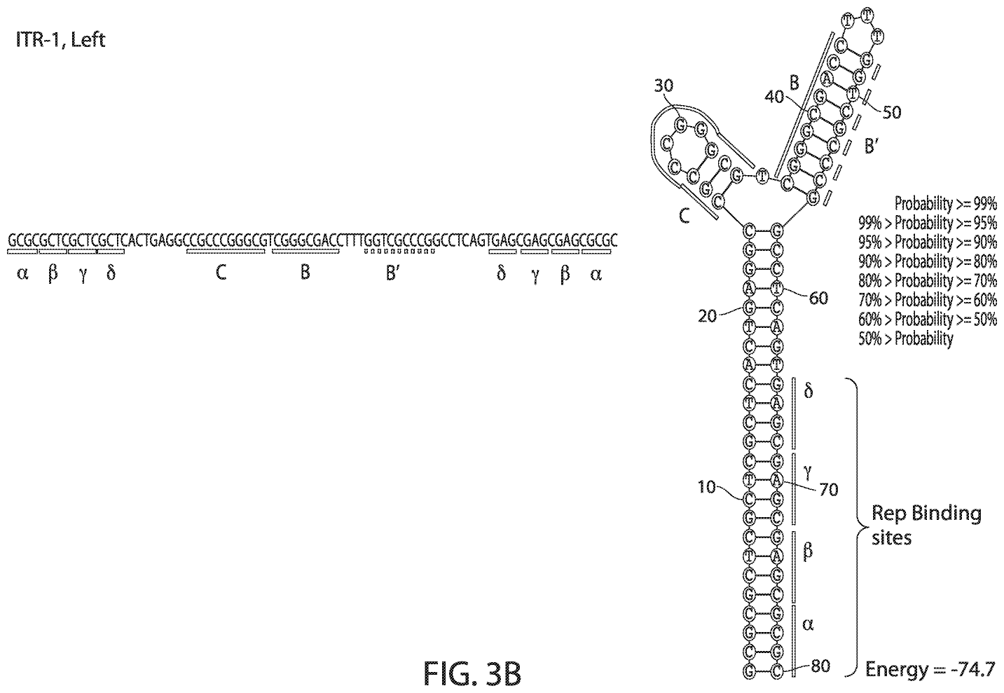

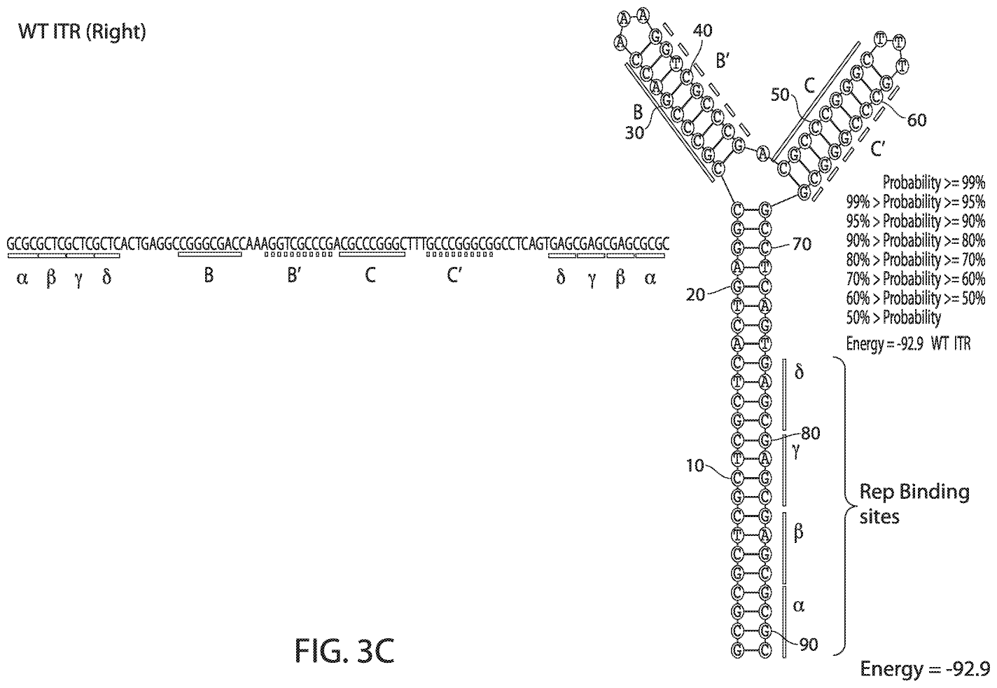

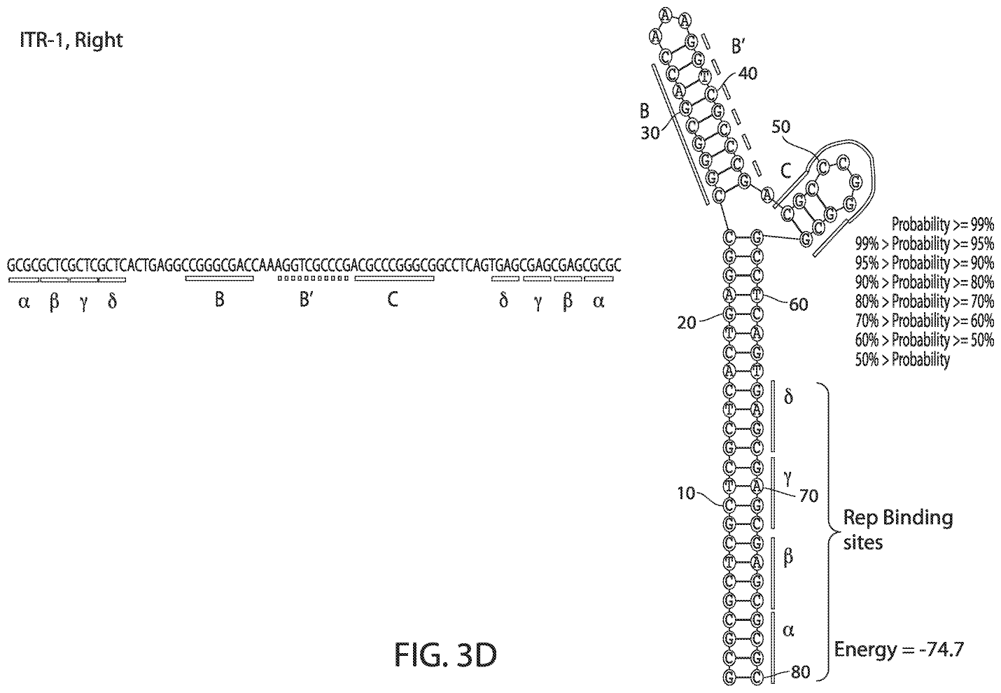

An ordinarily skilled artisan is aware that ITR sequences have a common structure of a double-stranded Holliday junction, which typically is a T-shaped or Y-shaped hairpin structure (see e.g., FIG. 2A and FIG. 3A), where each WT-ITR is formed by two palindromic arms or loops (B-B′ and C-C′) embedded in a larger palindromic arm (A-A′), and a single stranded D sequence, (where the order of these palindromic sequences defines the flip or flop orientation of the ITR). See, for example, structural analysis and sequence comparison of ITRs from different AAV serotypes (AAV1-AAV6) and described in Grimm et al., J. Virology, 2006; 80(1); 426-439; Yan et al., J. Virology, 2005; 364-379; Duan et al., Virology 1999; 261; 8-14. One of ordinary skill in the art can readily determine WT-ITR sequences from any AAV serotype for use in a ceDNA vector or ceDNA-plasmid based on the exemplary AAV2 ITR sequences provided herein. See, for example, the sequence comparison of ITRs from different AAV serotypes (AAV1-AAV6, and avian AAV (AAAV) and bovine AAV (BAAV)) described in Grimm et al., J. Virology, 2006; 80(1); 426-439; that show the % identity of the left ITR of AAV2 to the left ITR from other serotypes: AAV-1 (84%), AAV-3 (86%), AAV-4 (79%), AAV-5 (58%), AAV-6 (left ITR) (100%) and AAV-6 (right ITR) (82%).

### A. Symmetrical ITR Pairs

In some embodiments, a ceDNA vector useful for insertion of a transgene into a GSH as described herein comprises, in the 5′ to 3′ direction: a first adeno-associated virus (AAV) inverted terminal repeat (ITR), a HA-L (or 5′ HA), a nucleotide sequence of interest (for example an expression cassette as described herein), a HA-R (or 3′ HA) and a second AAV ITR, where the first ITR (5′ ITR) and the second ITR (3′ ITR) are symmetric, or substantially symmetrical with respect to each other—that is, a ceDNA vector can comprise ITR sequences that have a symmetrical three-dimensional spatial organization such that their structure is the same shape in geometrical space, or have the same A, C-C′ and B-B′ loops in 3D space. In such an embodiment, a symmetrical ITR pair, or substantially symmetrical ITR pair can be modified ITRs (e.g., mod-ITRs) that are not wild-type ITRs. A mod-ITR pair can have the same sequence which has one or more modifications from wild-type ITR and are reverse complements (inverted) of each other. In alternative embodiments, a modified ITR pair are substantially symmetrical as defined herein, that is, the modified ITR pair can have a different sequence but have corresponding or the same symmetrical three-dimensional shape.

(i) Wildtype ITRs

In some embodiments, the symmetrical ITRs, or substantially symmetrical ITRs are wild type (WT-ITRs) as described herein. That is, both ITRs have a wild type sequence, but do not necessarily have to be WT-ITRs from the same AAV serotype. That is, in some embodiments, one WT-ITR can be from one AAV serotype, and the other WT-ITR can be from a different AAV serotype. In such an embodiment, a WT-ITR pair are substantially symmetrical as defined herein, that is, they can have one or more conservative nucleotide modification while still retaining the symmetrical three-dimensional spatial organization.

Accordingly, as disclosed herein, a ceDNA vector useful for insertion of a transgene into a GSH can contain a transgene or heterologous nucleic acid sequence positioned between a HA-L and HA-R, which is flanked by two wild-type inverted terminal repeat (WT-ITR) sequences, that are either the reverse complement (inverted) of each other, or alternatively, are substantially symmetrical relative to each other—that is a WT-ITR pair have symmetrical three-dimensional spatial organization. In some embodiments, a wild-type ITR sequence (e.g. AAV WT-ITR) comprises a functional Rep binding site (RBS; e.g. 5′-GCGCGCTCGCTCGCTC-3′ for AAV2, SEQ ID NO: 60) and a functional terminal resolution site (TRS; e.g. 5′-AGTT-3′, SEQ ID NO: 62).

In one aspect, ceDNA vectors useful for insertion of a transgene into a GSH are obtainable from a vector polynucleotide that encodes a heterologous nucleic acid operatively positioned between a HA-L and a HA-R, which is flanked between two WT inverted terminal repeat sequences (WT-ITRs) (e.g. AAV WT-ITRs). That is, both ITRs have a wild type sequence, but do not necessarily have to be WT-ITRs from the same AAV serotype. That is, in some embodiments, one WT-ITR can be from one AAV serotype, and the other WT-ITR can be from a different AAV serotype. In such an embodiment, the WT-ITR pair are substantially symmetrical as defined herein, that is, they can have one or more conservative nucleotide modification while still retaining the symmetrical three-dimensional spatial organization. In some embodiments, the 5′ WT-ITR is from one AAV serotype, and the 3′ WT-ITR is from the same or a different AAV serotype. In some embodiments, the 5′ WT-ITR and the 3′WT-ITR are mirror images of each other, that is they are symmetrical. In some embodiments, the 5′ WT-ITR and the 3′ WT-ITR are from the same AAV serotype.

WT ITRs are well known. In one embodiment the two ITRs are from the same AAV2 serotype. In certain embodiments one can use WT from other serotypes. There are a number of serotypes that are homologous, e.g. AAV2, AAV4, AAV6, AAV8. In one embodiment, closely homologous ITRs (e.g. ITRs with a similar loop structure) can be used. In another embodiment, one can use AAV WT ITRs that are more diverse, e.g., AAV2 and AAV5, and still another embodiment, one can use an ITR that is substantially WT—that is, it has the basic loop structure of the WT but some conservative nucleotide changes that do not alter or affect the properties. When using WT-ITRs from the same viral serotype, one or more regulatory sequences may further be used. In certain embodiments, the regulatory sequence is a regulatory switch that permits modulation of the activity of the ceDNA.

In some embodiments, one aspect of the technology described herein relates to a ceDNA vector, wherein the ceDNA vector comprises at least one heterologous nucleotide sequence, operably positioned between a HA-L and a HA-R, which is flanked between two wild-type inverted terminal repeat sequences (WT-ITRs), wherein the WT-ITRs can be from the same serotype, different serotypes or substantially symmetrical with respect to each other (i.e., have the symmetrical three-dimensional spatial organization such that their structure is the same shape in geometrical space, or have the same A, C-C′ and B-B′ loops in 3D space). In some embodiments, the symmetric WT-ITRs comprises a functional terminal resolution site and a Rep binding site. In some embodiments, the heterologous nucleic acid sequence encodes a transgene, and wherein the vector is not in a viral capsid.

In some embodiments, the WT-ITRs are the same but the reverse complement of each other. For example, the sequence AACG in the 5′ ITR may be CGTT (i.e., the reverse complement) in the 3′ ITR at the corresponding site. In one example, the 5′ WT-ITR sense strand comprises the sequence of ATCGATCG and the corresponding 3′ WT-ITR sense strand comprises CGATCGAT (i.e., the reverse complement of ATCGATCG). In some embodiments, the WT-ITRs ceDNA further comprises a terminal resolution site and a replication protein binding site (RPS) (sometimes referred to as a replicative protein binding site), e.g. a Rep binding site.

Exemplary WT-ITR sequences for use in the ceDNA vectors useful for insertion of a transgene into a GSH as disclosed herein comprises WT-ITRs are shown in Table 6 herein, which shows pairs of WT-ITRs (5′ WT-ITR and the 3′ WT-ITR).

As an exemplary example, the present disclosure provides a ceDNA vector for insertion of a transgene into a GSH comprising two ITRs that flank a HA-L and a HA-R, and located between the HA-L and HA-R is a promoter operably linked to a transgene (e.g., heterologous nucleic acid sequence), with or without the regulatory switch, where the ceDNA vector is devoid of capsid proteins and is: (a) produced from a ceDNA-plasmid (e.g., see FIGS. 1F-1G) that encodes WT-ITRs, where each WT-ITR has the same number of intramolecularly duplexed base pairs in its hairpin secondary configuration (preferably excluding deletion of any AAA or TTT terminal loop in this configuration compared to these reference sequences), and (b) is identified as ceDNA using the assay for the identification of ceDNA by agarose gel electrophoresis under native gel and denaturing conditions as discussed in Examples 1 and 5 herein.

In some embodiments, the flanking WT-ITRs are substantially symmetrical to each other. In this embodiment the 5′ WT-ITR can be from one serotype of AAV, and the 3′ WT-ITR from a different serotype of AAV, such that the WT-ITRs are not identical reverse complements. For example, the 5′ WT-ITR can be from AAV2, and the 3′ WT-ITR from a different serotype (e.g. AAV1, 3, 4, 5, 6, 7, 8, 9, 10, 11, and 12. In some embodiments, WT-ITRs can be selected from two different parvoviruses selected from any to of: AAV1, AAV2, AAV3, AAV4, AAV5, AAV6, AAV7, AAV8, AAV9, AAV10, AAV11, AAV12, AAV13, snake parvovirus (e.g., royal python parvovirus), bovine parvovirus, goat parvovirus, avian parvovirus, canine parvovirus, equine parvovirus, shrimp parvovirus, porcine parvovirus, or insect AAV. In some embodiments, such a combination of WT ITRs is the combination of WT-ITRs from AAV2 and AAV6. In one embodiment, the substantially symmetrical WT-ITRs are when one is inverted relative to the other ITR at least 90% identical, at least 95% identical, at least 96% . . . 97% . . . 98% . . . 99% . . . 99.5% and all points in between, and has the same symmetrical three-dimensional spatial organization. In some embodiments, a WT-ITR pair are substantially symmetrical as they have symmetrical three-dimensional spatial organization, e.g., have the same 3D organization of the A, C-C′. B-B′ and D arms. In one embodiment, a substantially symmetrical WT-ITR pair are inverted relative to the other, and are at least 95% identical, at least 96% . . . 97% . . . 98% . . . 99% . . . 99.5% and all points in between, to each other, and one WT-ITR retains the Rep-binding site (RBS) of 5′-GCGCGCTCGCTCGCTC-3′ (SEQ ID NO: 60) and a terminal resolution site (trs). In some embodiments, a substantially symmetrical WT-ITR pair are inverted relative to each other, and are at least 95% identical, at least 96% . . . 97% . . . 98% . . . 99% . . . 99.5% and all points in between, to each other, and one WT-ITR retains the Rep-binding site (RBS) of 5′-GCGCGCTCGCTCGCTC-3′ (SEQ ID NO: 60) and a terminal resolution site (trs) and in addition to a variable palindromic sequence allowing for hairpin secondary structure formation. Homology can be determined by standard means well known in the art such as BLAST (Basic Local Alignment Search Tool), BLASTN at default setting.

In some embodiments, the structural element of the ITR can be any structural element that is involved in the functional interaction of the ITR with a large Rep protein (e.g., Rep 78 or Rep 68). In certain embodiments, the structural element provides selectivity to the interaction of an ITR with a large Rep protein, i.e., determines at least in part which Rep protein functionally interacts with the ITR. In other embodiments, the structural element physically interacts with a large Rep protein when the Rep protein is bound to the ITR. Each structural element can be, e.g., a secondary structure of the ITR, a nucleotide sequence of the ITR, a spacing between two or more elements, or a combination of any of the above. In one embodiment, the structural elements are selected from the group consisting of an A and an A′ arm, a B and a B′ arm, a C and a C′ arm, a D arm, a Rep binding site (RBE) and an RBE′ (i.e., complementary RBE sequence), and a terminal resolution sire (trs).

By way of example only, Table 5 indicates exemplary combinations of WT-ITRs.

Table 5: Exemplary combinations of WT-ITRs from the same serotype or different serotypes, or different parvoviruses. The order shown is not indicative of the ITR position, for example, “AAV1, AAV2” demonstrates that the ceDNA can comprise a WT-AAV1 ITR in the 5′ position, and a WT-AAV2 ITR in the 3′ position, or vice versa, a WT-AAV2 ITR the 5′ position, and a WT-AAV1 ITR in the 3′ position. Abbreviations: AAV serotype 1 (AAV1), AAV serotype 2 (AAV2), AAV serotype 3 (AAV3), AAV serotype 4 (AAV4), AAV serotype 5 (AAV5), AAV serotype 6 (AAV6), AAV serotype 7 (AAV7), AAV serotype 8 (AAV8), AAV serotype 9 (AAV9), AAV serotype 10 (AAV10), AAV serotype 11 (AAV11), or AAV serotype 12 (AAV12); AAVrh8, AAVrh10, AAV-DJ, and AAV-DJ8 genome (E.g., NCBI: NC_002077; NC 001401; NC001729; NC001829; NC006152; NC_006260; NC_006261), ITRs from warm-blooded animals (avian AAV (AAAV), bovine AAV (BAAV), canine, equine, and ovine AAV), ITRs from B19 parvoviris (GenBank Accession No: NC_000883), Minute Virus from Mouse (MVM) (GenBank Accession No. NC 001510); Goose: goose parvovirus (GenBank Accession No. NC_001701); snake: snake parvovirus 1 (GenBank Accession No. NC_006148).

TABLE 5

| | | | | |
|---|---|---|---|---|
| AAV1, | AAV2, | AAV3, | AAV4, | AAV5, |
| AAV1 | AAV2 | AAV3 | AAV4 | AAV5 |
| AAV1, | AAV2, | AAV3, | AAV4, | AAV5, |
| AAV2 | AAV3 | AAV4 | AAV5 | AAV6 |
| AAV1, | AAV2, | AAV3, | AAV4, | AAV5, |
| AAV3 | AAV4 | AAV5 | AAV6 | AAV7 |
| AAV1, | AAV2, | AAV3, | AAV4, | AAV5, |
| AAV4 | AAV5 | AAV6 | AAV7 | AAV8 |
| AAV1, | AAV2, | AAV3, | AAV4, | AAV5, |
| AAV5 | AAV6 | AAV7 | AAV8 | AAV9 |
| AAV1, | AAV2, | AAV3, | AAV4, | AAV5 , |
| AAV6 | AAV7 | AAV8 | AAV9 | AAV10 |
| AAV1, | AAV2, | AAV3, | AAV4, | AAV5, |
| AAV7 | AAV8 | AAV9 | AAV10 | AAV11 |
| AAV1, | AAV2, | AAV3, | AAV4, | AAV5, |
| AAV8 | AAV9 | AAV10 | AAV11 | AAV12 |
| AAV1, | AAV2, | AAV3, | AAV4, | AAV5, |
| AAV9 | AAV10 | AAV11 | AAV12 | AAVRH8 |
| AAV1, | AAV2, | AAV3, | AAV4, | AAV5, |
| AAV10 | AAV11 | AAV12 | AAVRH8 | AAVRH10 |
| AAV1, | AAV2, | AAV3, | AAV4, | AAV5, |
| AAV11 | AAV12 | AAVRH8 | AAVRH10 | AAV13 |
| AAV1, | AAV2, | AAV3, | AAV4, | AAV5, |
| AAV12 | AAVRH8 | AAVRH10 | AAV13 | AAVDJ |
| AAV1, | AAV2, | AAV3, | AAV4, | AAV5, |
| AAVRH8 | AAVRH10 | AAV13 | AAVDJ | AAVDJ8 |
| AAV1, | AAV2, | AAV3, | AAV4, | AAV5, |
| AAVRH10 | AAV13 | AAVDJ | AAVDJ8 | AVIAN |
| AAV1, | AAV2, | AAV3, | AAV4, | AAV5, |
| AAV13 | AAVDJ | AAVDJ8 | AVIAN | BOVINE |
| AAV1, | AAV2, | AAV3, | AAV4, | AAV5, |
| AAVDJ | AAVDJ8 | AVIAN | BOVINE | CANINE |
| AAV1, | AAV2, | AAV3, | AAV4, | AAV5, |
| AAVDJ8 | AVIAN | BOVINE | CANINE | EQUINE |
| AAV1, | AAV2, | AAV3, | AAV4, | AAV5, |
| AVIAN | BOVINE | CANINE | EQUINE | GOAT |
| AAV1, | AAV2, | AAV3, | AAV4, | AAV5, |
| BOVINE | CANINE | EQUINE | GOAT | SHRIMP |
| AAV1, | AAV2, | AAV3, | AAV4, | AAV5, |
| CANINE | EQUINE | GOAT | SHRIMP | PORCINE |
| AAV1, | AAV2, | AAV3, | AAV4, | AAV5, |
| EQUINE | GOAT | SHRIMP | PORCINE | INSECT |
| AAV1, | AAV2, | AAV3, | AAV4, | AAV5, |
| GOAT | SHRIMP | PORCINE | INSECT | OVINE |
| AAV1, | AAV2, | AAV3, | AAV4, | AAV5,B19 |
| SHRIMP | PORCINE | INSECT | OVINE | |
| AAV1, | AAV2, | AAV3, | AAV4, | AAV5, |
| PORCINE | INSECT | OVINE | B19 | MVM |
| AAV1, | AAV2, | AAV3, | AAV4, | AAV5, |
| INSECT | OVINE | B19 | MVM | GOOSE |
| AAV1, | AAV2, | AAV3, | AAV4, | AAV5, |
| OVINE | B19 | MVM | GOOSE | SNAKE |
| AAV1, | AAV2, | AAV3, | AAV4, | |
| B19 | MVM | GOOSE | SNAKE | |
| AAV1, | AAV2, | AAV3, | | |
| MVM | GOOSE | SNAKE | | |
| AAV1, | AAV2, | | | |
| GOOSE | SNAKE | | | |
| AAV1, | | | | |
| SNAKE | | | | |
| AAV6, | AAV7, | AAV8, | AAV9, | AAV10, |
| AAV6 | AAV7 | AAV8 | AAV9 | AAV10 |
| AAV6, | AAV7, | AAV8, | AAV9, | AAV10, |
| AAV7 | AAV8 | AAV9 | AAV10 | AAV11 |
| AAV6, | AAV7, | AAV8, | AAV9, | AAV10, |
| AAV8 | AAV9 | AAV10 | AAV11 | AAV12 |
| AAV6, | AAV7, | AAV8, | AAV9, | AAV10, |
| AAV9 | AAV10 | AAV11 | AAV12 | AAVRH8 |
| AAV6, | AAV7, | AAV8, | AAV9, | AAV10, |
| AAV10 | AAV11 | AAV12 | AAVRH8 | AAVRH10 |
| AAV6, | AAV7, | AAV8, | AAV9, | AAV10, |
| AAV11 | AAV12 | AAVRH8 | AAVRH10 | AAV13 |
| AAV6, | AAV7, | AAV8, | AAV9, | AAV10, |
| AAV12 | AAVRH8 | AAVRH10 | AAV13 | AAVDJ |
| AAV6, | AAV7, | AAV8, | AAV9, | AAV10, |
| AAVRH8 | AAVRH10 | AAV13 | AAVDJ | AAVDJ8 |
| AAV6, | AAV7, | AAV8, | AAV9, | AAV10, |
| AAVRH10 | AAV13 | AAVDJ | AAVDJ8 | AVIAN |
| AAV6, | AAV7, | AAV8, | AAV9, | AAV10, |
| AAV13 | AAVDJ | AAVDJ8 | AVIAN | BOVINE |
| AAV6, | AAV7, | AAV8, | AAV9, | AAV10, |
| AAVDJ | AAVDJ8 | AVIAN | BOVINE | CANINE |
| AAV6, | AAV7, | AAV8, | AAV9, | AAV10, |
| AAVDJ8 | AVIAN | BOVINE | CANINE | EQUINE |
| AAV6, | AAV7, | AAV8, | AAV9, | AAV10, |
| AVIAN | BOVINE | CANINE | EQUINE | GOAT |
| AAV6, | AAV7, | AAV8, | AAV9, | AAV10, |
| BOVINE | CANINE | EQUINE | GOAT | SHRIMP |
| AAV6, | AAV7, | AAV8, | AAV9, | AAV10, |
| CANINE | EQUINE | GOAT | SHRIMP | PORCINE |
| AAV6, | AAV7, | AAV8, | AAV9, | AAV10, |
| EQUINE | GOAT | SHRIMP | PORCINE | INSECT |
| AAV6, | AAV7, | AAV8, | AAV9, | AAV10, |
| GOAT | SHRIMP | PORCINE | INSECT | OVINE |
| AAV6, | AAV7, | AAV8, | AAV9, | AAV10, |
| SHRIMP | PORCINE | INSECT | OVINE | B19 |
| AAV6, | AAV7, | AAV8, | AAV9,B19 | AAV10, |
| PORCINE | INSECT | OVINE | | MVM |
| AAV6, | AAV7, | AAV8,B19 | AAV9, | AAV10, |
| INSECT | OVINE | | MVM | GOOSE |
| AAV6, | AAV7,B19 | AAV8, | AAV9, | AAV10, |
| OVINE | | MVM | GOOSE | SNAKE |
| AAV6,B19 | AAV7, | AAV8, | AAV9, | |
| | MVM | GOOSE | SNAKE | |
| AAV6, | AAV7, | AAV8, | | |
| MVM | GOOSE | SNAKE | | |
| AAV6, | AAV7, | | | |
| GOOSE | SNAKE | | | |
| AAV6, | | | | |
| SNAKE | | | | |
| AAV11, | AAV12, | AAVRH8, | AAVRH10, | AAV13, |
| AAV11 | AAV12 | AAVRH8 | AAVRH10 | AAV13 |
| AAV11, | AAV12, | AAVRH8, | AAVRH10, | AAV13, |
| AAV12 | AAVRH8 | AAVRH10 | AAV13 | AAVDJ |
| AAV11, | AAV12, | AAVRH8, | AAVRH10, | AAV13, |
| AAVRH8 | AAVRH10 | AAV13 | AAVDJ | AAVDJ8 |
| AAV11, | AAV12, | AAVRH8, | AAVRH10, | AAV13, |
| AAVRH10 | AAV13 | AAVDJ | AAVDJ8 | AVIAN |
| AAV11, | AAV12, | AAVRH8, | AAVRH10, | AAV13, |
| AAV13 | AAVDJ | AAVDJ8 | AVIAN | BOVINE |
| AAV11, | AAV12, | AAVRH8, | AAVRH10, | AAV13, |
| AAVDJ | AAVDJ8 | AVIAN | BOVINE | CANINE |
| AAV11, | AAV12, | AAVRH8, | AAVRH10, | AAV13, |
| AAVDJ8 | AVIAN | BOVINE | CANINE | EQUINE |
| AAV11, | AAV12, | AAVRH8, | AAVRH10, | AAV13, |
| AVIAN | BOVINE | CANINE | EQUINE | GOAT |
| AAV11, | AAV12, | AAVRH8, | AAVRH10, | AAV13, |
| BOVINE | CANINE | EQUINE | GOAT | SHRIMP |
| AAV11, | AAV12, | AAVRH8, | AAVRH10, | AAV13, |
| CANINE | EQUINE | GOAT | SHRIMP | PORCINE |
| AAV11, | AAV12, | AAVRH8, | AAVRH10, | AAV13, |
| EQUINE | GOAT | SHRIMP | PORCINE | INSECT |
| AAV11, | AAV12, | AAVRH8, | AAVRH10, | AAV13, |
| GOAT | SHRIMP | PORCINE | INSECT | OVINE |
| AAV11, | AAV12, | AAVRH8, | AAVRH10, | AAV13, |
| SHRIMP | PORCINE | INSECT | OVINE | B19 |
| AAV11, | AAV12, | AAVRH8, | AAVRH10, | AAV13, |
| PORCINW | INSECT | OVINE | B19 | MVM |
| AAV11, | AAV12, | AAVRH8, | AAVRH10, | AAV13, |
| INSECT | OVINE | B19 | MVM | GOOSE |
| AAV11, | AAV12, | AAVRH8, | AAVRH10, | AAV13, |
| OVINE | B19 | MVM | GOOSE | SNAKE |
| AAV11, | AAV12, | AAVRH8, | AAVRH10, | |
| B19 | MVM | GOOSE | SNAKE | |
| AAV11, | AAV12, | AAVRH8, | | |
| MVM | GOOSE | SNAKE | | |
| AAV11, | AAV12, | | | |
| GOOSE | SNAKE | | | |
| AAV11, | | | | |
| SNAKE | | | | |
| AAVDJ, | AAVDJ8, | AVIAN, | BOVINE, | CANINE, |
| AAVDJ | AVVDJ8 | AVIAN | BOVINE | CANINE |
| AAVDJ, | AAVDJ8, | AVIAN, | BOVINE, | CANINE, |
| AAVDJ8 | AVIAN | BOVINE | CANINE | EQUINE |
| AAVDJ, | AAVDJ8, | AVIAN, | BOVINE, | CANINE, |
| AVIAN | BOVINE | CANINE | EQUINE | GOAT |
| AAVDJ, | AAVDJ8, | AVIAN, | BOVINE, | CANINE, |
| BOVINE | CANINE | EQUINE | GOAT | SHRIMP |
| AAVDJ, | AAVDJ8, | AVIAN, | BOVINE, | CANINE, |
| CANINE | EQUINE | GOAT | SHRIMP | PORCINE |
| AAVDJ, | AAVDJ8, | AVIAN, | BOVINE, | CANINE, |
| EQUINE | GOAT | SHRIMP | PORCINE | INSECT |
| AAVDJ, | AAVDJ8, | AVIAN, | BOVINE, | CANINE, |
| GOAT | SHRIMP | PORCINE | INSECT | OVINE |
| AAVDJ, | AAVDJ8, | AVIAN, | BOVINE, | CANINE, |
| SHRIMP | PORCINE | INSECT | OVINE | B19 |
| AAVDJ, | AAVDJ8, | AVIAN, | BOVINE, | CANINE, |
| PORCINE | INSECT | OVINE | B19 | MVM |
| AAVDJ, | AAVDJ8, | AVIAN, | BOVINE, | CANINE, |
| INSECT | OVINE | B19 | MVM | GOOSE |
| AAVDJ, | AAVDJ8, | AVIAN, | BOVINE, | CANINE, |
| OVINE | B19 | MVM | GOOSE | SNAKE |
| AAVDJ, | AAVDJ8, | AVIAN, | BOVINE, | |
| B19 | MVM | GOOSE | SNAKE | |
| AAVDJ, | AAVDJ8, | AVIAN, | | |
| MVM | GOOSE | SNAKE | | |
| AAVDJ, | AAVDJ8, | | | |
| GOOSE | SNAKE | | | |
| AAVDJ, | | | | |
| SNAKE | | | | |
| EQUINE, | GOAT, | SHRIMP, | PORCINE, | INSECT, |
| EQUINE | GOAT | SHRIMP | PORCINE | INSECT |
| EQUINE, | GOAT, | SHRIMP, | PORCINE, | INSECT, |
| GOAT | SHRIMP | PORCINE | INSECT | OVINE |
| EQUINE, | GOAT, | SHRIMP, | PORCINE, | INSECT,B19 |
| SHRIMP | PORCINE | INSECT | OVINE | |
| EQUINE, | GOAT, | SHRIMP, | PORCINE, | INSECT, |
| PORCINE | INSECT | OVINE | B19 | MVM |
| EQUINE, | GOAT, | SHRIMP, | PORCINE, | INSECT, |
| INSECT | OVINE | B19 | MVM | GOOSE |
| EQUINE, | GOAT, | SHRIMP, | PORCINE, | INSECT, |
| OVINE | B19 | MVM | GOOSE | SNAKE |
| EQUINE, | GOAT, | SHRIMP, | PORCINE, | |
| B19 | MVM | GOOSE | SNAKE | |
| EQUINE, | GOAT, | SHRIMP, | | |
| MVM | GOOSE | SNAKE | | |
| EQUINE, | GOAT, | | | |
| GOOSE | SNAKE | | | |
| EQUINE, | | | | |
| SNAKE | | | | |
| OVINE, | B19, B19 | MVM, | GOOSE, | SNAKE, |
| OVINE | | MVM | GOOSE | SNAKE |
| OVINE, | B19, | MVM, | GOOSE, | |
| B19 | MVM | GOOSE | SNAKE | |
| OVINE, | B19, | MVM, | | |
| MVM | GOOSE | SNAKE | | |
| OVINE, | B19, | | | |
| GOOSE | SNAKE | | | |
| OVINE, | | | | |
| SNAKE | | | | |

By way of example only, Table 6 shows the sequences of exemplary WT-ITRs from some different AAV serotypes.

TABLE 6

| AAV | | |
|---|---|---|
| serotype | 5′ WT-ITR (LEFT) | 3′ WT-ITR (RIGHT) |
| AAV1 | 5′- | 5′- |
| | TTGCCCACTCCCTCTCTGCGCGCTCGCTCGCTC | TTACCCTAGTGATGGAGTTGCCCACTC |
| | GGTGGGGCCTGCGGACCAAAGGTCCGCAGAC | CCTCTCTGCGCGCGTCGCTCGCTCGGT |
| | GGCAGAGGTCTCCTCTGCCGGCCCCACCGAGC | GGGGCCGGCAGAGGAGACCTCTGCCG |
| | GAGCGACGCGCGCAGAGAGGGAGTGGGCAA | TCTGCGGACCTTTGGTCCGCAGGCCCC |
| | CTCCATCACTAGGGTAA-3′ | ACCGAGCGAGCGAGCGCGCAGAGAGG |
| | (SEQ ID NO: 5) | GAGTGGGCAA-3′ (SEQ ID NO: 10) |
| AAV2 | CCTGCAGGCAGCTGCGCGCTCGCTCG | AGGAACCCCTAGTGATGGAGTTGGCCA |
| | CTCACTGAGGCCGCCCGGGCAAAGCC | CTCCCTCTCTGCGCGCTCGCTCGCTCAC |
| | CGGGCGTCGGGCGACCTTTGGTCGCC | TGAGGCCGGGCGACCAAAGGTCGCCC |
| | CGGCCTCAGTGAGCGAGCGAGCGCGC | GACGCCCGGGCTTTGCCCGGGCGGCCT |
| | AGAGAGGGAGTGGCCAACTCCATCAC | CAGTGAGCGAGCGAGCGCGCAGCTGC |
| | TAGGGGTTCCT (SEQ ID NO: 2) | CTGCAGG (SEQ ID NO: 1) |
| AAV3 | 5′- | 5′- |
| | TTGGCCACTCCCTCTATGCGCACTCGC | ATACCTCTAGTGATGGAGTTGGCCACT |
| | TCGCTCGGTGGGGCCTGGCGACCAAA | CCCTCTATGCGCACTCGCTCGCTCGGT |
| | GGTCGCCAGACGGACGTGGGTTTCCA | GGGGCCGGACGTGGAAACCCACGTCC |
| | CGTCCGGCCCCACCGAGCGAGCGAGT | GTCTGGCGACCTTTGGTCGCCAGGCCC |
| | GCGCATAGAGGGAGTGGCCAACTCCA | CACCGAGCGAGCGAGTGCGCATAGAG |
| | TCACTAGAGGTAT-3′ (SEQ ID NO: 6) | GGAGTGGCCAA-3′ (SEQ ID NO: 11) |
| AAV4 | 5′- | 5′- |
| | TTGGCCACTCCCTCTATGCGCGCTCGC | AGTTGGCCACATTAGCTATGCGCGCTC |
| | TCACTCACTCGGCCCTGGAGACCAAA | GCTCACTCACTCGGCCCTGGAGACCAA |
| | GGTCTCCAGACTGCCGGCCTCTGGCC | AGGTCTCCAGACTGCCGGCCTCTGGCC |
| | GGCAGGGCCGAGTGAGTGAGCGAGC | GGCAGGGCCGAGTGAGTGAGCGAGCG |
| | GCGCATAGAGGGAGTGGCCAACT-3′ | CGCATAGAGGGAGTGGCCAA-3′ (SEQ |
| | (SEQ ID NO: 7) | ID NO: 12) |
| AAV5 | 5′- | 5′- |
| | TCCCCCCTGTCGCGTTCGCTCGCTCGCTGGCTC | CTTACAAAACCCCCTTGCTTGAGAGTG |
| | GTTTGGGGGGGCGACGGCCAGAGGGCCGTCG | TGGCACTCTCCCCCCTGTCGCGTTCGCT |
| | TCTGGCAGCTCTTTGAGCTGCCACCCCCCCAAA | CGCTCGCTGGCTCGTTTGGGGGGGTGG |
| | CGAGCCAGCGAGCGAGCGAACGCGACAGGG | CAGCTCAAAGAGCTGCCAGACGACGG |
| | GGGAGAGTGCCACACTCTCAAGCAAGGGGGT | CCCTCTGGCCGTCGCCCCCCCAAACGA |
| | TTTGTAAG-3′ (SEQ ID NO: 8) | GCCAGCGAGCGAGCGAACGCGACAGG |
| | | GGGGA-3′ (SEQ ID NO: 13) |
| AAV6 | 5′- | 5′- |
| | TTGCCCACTCCCTCTAATGCGCGCTCG | ATACCCCTAGTGATGGAGTTGCCCACT |
| | CTCGCTCGGTGGGGCCTGCGGACCAA | CCCTCTATGCGCGCTCGCTCGCTCGGT |
| | AGGTCCGCAGACGGCAGAGGTCTCCT | GGGGCCGGCAGAGGAGACCTCTGCCG |
| | CTGCCGGCCCCACCGAGCGAGCGAGC | TCTGCGGACCTTTGGTCCGCAGGCCCC |
| | GCGCATAGAGGGAGTGGGCAACTCCA | ACCGAGCGAGCGAGCGCGCATTAGAG |
| | TCACTAGGGGTAT-3′ (SEQ ID NO: 9) | GGAGTGGGCAA (SEQ ID NO: 14) |

In some embodiments, the nucleotide sequence of the WT-ITR sequence can be modified (e.g., by modifying 1, 2, 3, 4 or 5, or more nucleotides or any range therein), whereby the modification is a substitution for a complementary nucleotide, e.g., G for a C, and vice versa, and T for an A, and vice versa.

In certain embodiments of the present invention, the synthetically produced ceDNA vector does not have a WT-ITR consisting of the nucleotide sequence selected from any of: SEQ ID NOs: 1, 2, 5-14. In alternative embodiments of the present invention, if a ceDNA vector has a WT-ITR comprising the nucleotide sequence selected from any of: SEQ ID NOs: 1, 2, 5-14, then the flanking ITR is also WT and the ceDNA vector comprises a regulatory switch, e.g., as disclosed herein and in International application PCT/US18/49996 (e.g., see Table 11 of PCT/US18/49996). In some embodiments, the ceDNA vector comprises a regulatory switch as disclosed herein and a WT-ITR selected having the nucleotide sequence selected from any of the group consisting of: SEQ ID NO: 1, 2, 5-14.

The ceDNA vector described herein can include WT-ITR structures that retains an operable RBE, trs and RBE′ portion. FIG. 2A and FIG. 2B, using wild-type ITRs for exemplary purposes, show one possible mechanism for the operation of a trs site within a wild type ITR structure portion of a ceDNA vector. In some embodiments, the ceDNA vector contains one or more functional WT-ITR polynucleotide sequences that comprise a Rep-binding site (RBS; 5′-GCGCGCTCGCTCGCTC-3′ (SEQ ID NO: 60) for AAV2) and a terminal resolution site (TRS; 5′-AGTT (SEQ ID NO: 62)). In some embodiments, at least one WT-ITR is functional. In alternative embodiments, where a ceDNA vector comprises two WT-ITRs that are substantially symmetrical to each other, at least one WT-ITR is functional and at least one WT-ITR is non-functional.

B. Modified ITRs (Mod-ITRs) in General for ceDNA Vectors for Insertion of a Transgene at a GSH Locus Comprising Asymmetric ITR Pairs or Symmetric ITR Pairs

As discussed herein, a ceDNA vector for insertion of a transgene into a GSH can comprise a symmetrical ITR pair or an asymmetrical ITR pair. In both instances, one or both of the ITRs can be modified ITRs—the difference being that in the first instance (i.e., symmetric mod-ITRs), the mod-ITRs have the same three-dimensional spatial organization (i.e., have the same A-A′, C-C′ and B-B′ arm configurations), whereas in the second instance (i.e., asymmetric mod-ITRs), the mod-ITRs have a different three-dimensional spatial organization (i.e., have a different configuration of A-A′, C-C′ and B-B′ arms).

In some embodiments, a modified ITR is an ITRs that is modified by deletion, insertion, and/or substitution as compared to a wild-type ITR sequence (e.g. AAV ITR). In some embodiments, at least one of the ITRs in the ceDNA vector comprises a functional Rep binding site (RBS; e.g. 5′-GCGCGCTCGCTCGCTC-3′ for AAV2, SEQ ID NO: 60) and a functional terminal resolution site (TRS; e.g. 5′-AGTT-3′, SEQ ID NO: 62.) In one embodiment, at least one of the ITRs is a non-functional ITR. In one embodiment, the different or modified ITRs are not each wild type ITRs from different serotypes.

Specific alterations and mutations in the ITRs are described in detail herein, but in the context of ITRs, “altered” or “mutated” or “modified”, it indicates that nucleotides have been inserted, deleted, and/or substituted relative to the wild-type, reference, or original ITR sequence. The altered or mutated ITR can be an engineered ITR. As used herein, “engineered” refers to the aspect of having been manipulated by the hand of man. For example, a polypeptide is considered to be “engineered” when at least one aspect of the polypeptide, e.g., its sequence, has been manipulated by the hand of man to differ from the aspect as it exists in nature.

In some embodiments, a mod-ITR may be synthetic. In one embodiment, a synthetic ITR is based on ITR sequences from more than one AAV serotype. In another embodiment, a synthetic ITR includes no AAV-based sequence. In yet another embodiment, a synthetic ITR preserves the ITR structure described above although having only some or no AAV-sourced sequence. In some aspects, a synthetic ITR may interact preferentially with a wild type Rep or a Rep of a specific serotype, or in some instances will not be recognized by a wild-type Rep and be recognized only by a mutated Rep.

The skilled artisan can determine the corresponding sequence in other serotypes by known means. For example, determining if the change is in the A, A′, B, B′, C, C′ or D region and determine the corresponding region in another serotype. One can use BLAST® (Basic Local Alignment Search Tool) or other homology alignment programs at default status to determine the corresponding sequence. The invention further provides populations and pluralities of ceDNA vectors for insertion of one or more transgenes into a GSH, where the ceDNA vector comprises mod-ITRs from a combination of different AAV serotypes—that is, one mod-ITR can be from one AAV serotype and the other mod-ITR can be from a different serotype. Without wishing to be bound by theory, in one embodiment one ITR can be from or based on an AAV2 ITR sequence and the other ITR of the ceDNA vector can be from or be based on any one or more ITR sequence of AAV serotype 1 (AAV1), AAV serotype 4 (AAV4), AAV serotype 5 (AAV5), AAV serotype 6 (AAV6), AAV serotype 7 (AAV7), AAV serotype 8 (AAV8), AAV serotype 9 (AAV9), AAV serotype 10 (AAV10), AAV serotype 11 (AAV11), or AAV serotype 12 (AAV12).

Any parvovirus ITR can be used as an ITR or as a base ITR for modification. Preferably, the parvovirus is a dependovirus. More preferably AAV. The serotype chosen can be based upon the tissue tropism of the serotype. AAV2 has a broad tissue tropism, AAV1 preferentially targets to neuronal and skeletal muscle, and AAV5 preferentially targets neuronal, retinal pigmented epithelia, and photoreceptors. AAV6 preferentially targets skeletal muscle and lung. AAV8 preferentially targets liver, skeletal muscle, heart, and pancreatic tissues. AAV9 preferentially targets liver, skeletal and lung tissue. In one embodiment, the modified ITR is based on an AAV2 ITR.

More specifically, the ability of a structural element to functionally interact with a particular large Rep protein can be altered by modifying the structural element. For example, the nucleotide sequence of the structural element can be modified as compared to the wild-type sequence of the ITR. In one embodiment, the structural element (e.g., A arm, A′ arm, B arm, B′ arm, C arm, C′ arm, D arm, RBE, RBE′, and trs) of an ITR can be removed and replaced with a wild-type structural element from a different parvovirus. For example, the replacement structure can be from AAV1, AAV2, AAV3, AAV4, AAV5, AAV6, AAV7, AAV8, AAV9, AAV10, AAV11, AAV12, AAV13, snake parvovirus (e.g., royal python parvovirus), bovine parvovirus, goat parvovirus, avian parvovirus, canine parvovirus, equine parvovirus, shrimp parvovirus, porcine parvovirus, or insect AAV. For example, the ITR can be an AAV2 ITR and the A or A′ arm or RBE can be replaced with a structural element from AAV5. In another example, the ITR can be an AAV5 ITR and the C or C′ arms, the RBE, and the trs can be replaced with a structural element from AAV2. In another example, the AAV ITR can be an AAV5 ITR with the B and B′ arms replaced with the AAV2 ITR B and B′ arms.

By way of example only, Table 7 indicates exemplary modifications of at least one nucleotide (e.g., a deletion, insertion and/or substitution) in regions of a modified ITR, where X is indicative of a modification of at least one nucleic acid (e.g., a deletion, insertion and/or substitution) in that section relative to the corresponding wild-type ITR. In some embodiments, any modification of at least one nucleotide (e.g., a deletion, insertion and/or substitution) in any of the regions of C and/or C′ and/or B and/or B′ retains three sequential T nucleotides (i.e., TTT) in at least one terminal loop. For example, if the modification results in any of: a single arm ITR (e.g., single C-C′ arm, or a single B-B′ arm), or a modified C-B′ arm or C′-B arm, or a two arm ITR with at least one truncated arm (e.g., a truncated C-C′ arm and/or truncated B-B′ arm), at least the single arm, or at least one of the arms of a two arm ITR (where one arm can be truncated) retains three sequential T nucleotides (i.e., TTT) in at least one terminal loop. In some embodiments, a truncated C-C′ arm and/or a truncated B-B′ arm has three sequential T nucleotides (i.e., TTT) in the terminal loop.

TABLE 7
Exemplary combinations of modifications of at least one
nucleotide (e.g., a deletion, insertion and/ or substitution)
to different B-B' and C-C' regions or arms of ITRs (X
indicates a nucleotide modification, e.g., addition, deletion
or substitution of at least one nucleotide in the region).

| B region | B’ region | C region | C’ region |
|---|---|---|---|
| X | | | |
| | X | | |
| X | X | | |
| | | X | |
| | | | X |
| | | X | X |
| X | | X | |
| X | | | X |
| | X | X | |
| | X | | X |
| X | X | X | |
| X | X | | X |
| X | | X | X |
| | X | X | X |
| X | X | X | X |

In some embodiments, mod-ITR for use in a ceDNA vector comprising an asymmetric ITR pair, or a symmetric mod-ITR pair as disclosed herein can comprise any one of the combinations of modifications shown in Table 7, and also a modification of at least one nucleotide in any one or more of the regions selected from: between A′ and C, between C and C′, between C′ and B, between B and B′ and between B′ and A. In some embodiments, any modification of at least one nucleotide (e.g., a deletion, insertion and/or substitution) in the C or C′ or B or B′ regions, still preserves the terminal loop of the stem-loop. In some embodiments, any modification of at least one nucleotide (e.g., a deletion, insertion and/or substitution) between C and C′ and/or B and B′ retains three sequential T nucleotides (i.e., TTT) in at least one terminal loop. In alternative embodiments, any modification of at least one nucleotide (e.g., a deletion, insertion and/or substitution) between C and C′ and/or B and B′ retains three sequential A nucleotides (i.e., AAA) in at least one terminal loop In some embodiments, a modified ITR for use herein can comprise any one of the combinations of modifications shown in Table 7, and also a modification of at least one nucleotide (e.g., a deletion, insertion and/or substitution) in any one or more of the regions selected from: A′, A and/or D. For example, in some embodiments, a modified ITR for use herein can comprise any one of the combinations of modifications shown in Table 7, and also a modification of at least one nucleotide (e.g., a deletion, insertion and/or substitution) in the A region. In some embodiments, a modified ITR for use herein can comprise any one of the combinations of modifications shown in Table 7, and also a modification of at least one nucleotide (e.g., a deletion, insertion and/or substitution) in the A′ region. In some embodiments, a modified ITR for use herein can comprise any one of the combinations of modifications shown in Table 7, and also a modification of at least one nucleotide (e.g., a deletion, insertion and/or substitution) in the A and/or A′ region. In some embodiments, a modified ITR for use herein can comprise any one of the combinations of modifications shown in Table 7, and also a modification of at least one nucleotide (e.g., a deletion, insertion and/or substitution) in the D region.

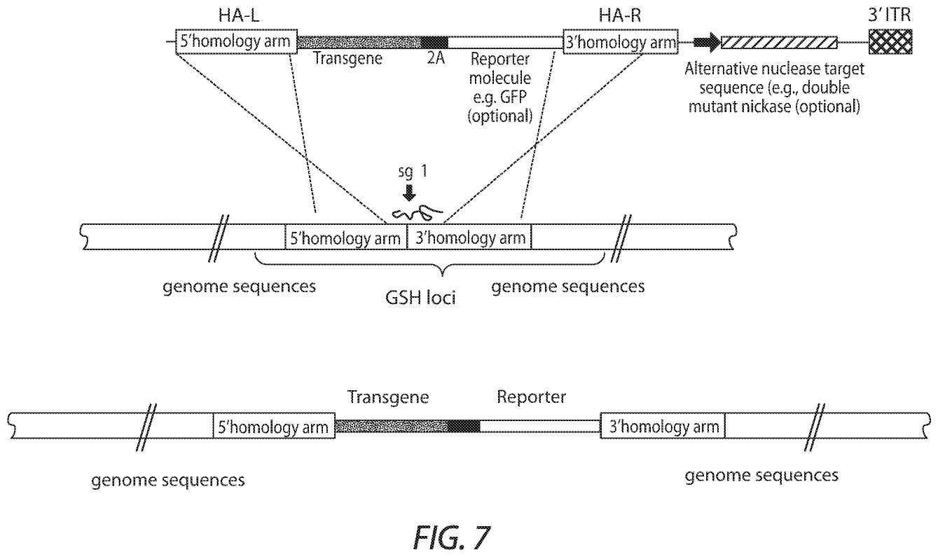

In one embodiment, the nucleotide sequence of the structural element can be modified (e.g., by modifying 1, 2, 3, 4, 5, 6, 7, 8, 9, 10, 11, 12, 13, 14, 15, 16, 17, 18, 19, or 20 or more nucleotides or any range therein) to produce a modified structural element. In one embodiment, the specific modifications to the ITRs are exemplified herein (e.g., SEQ ID NOS: 3, 4, 15-47, 101-116 or 165-187, or shown in FIG. 7A-7B of PCT/US2018/064242, filed on Dec. 6, 2018 (e.g., SEQ ID Nos 97-98, 101-103, 105-108, 111-112, 117-134, 545-54 in PCT/US2018/064242). In some embodiments, an ITR can be modified (e.g., by modifying 1, 2, 3, 4, 5, 6, 7, 8, 9, 10, 11, 12, 13, 14, 15, 16, 17, 18, 19, or 20 or more nucleotides or any range therein). In other embodiments, the ITR can have at least 80%, at least 85%, at least 90%, at least 95%, at least 96%, at least 97%, at least 98%, at least 99%, or more sequence identity with one of the modified ITRs of SEQ ID NOS: 3, 4, 15-47, 101-116 or 165-187, or the RBE-containing section of the A-A′ arm and C-C′ and B-B′ arms of SEQ ID NO: 3, 4, 15-47, 101-116 or 165-187, or shown in Tables 2-9 (i.e., SEQ ID NO: 110-112, 115-190, 200-468) of International application PCT/US18/49996, which is incorporated herein in its entirety by reference.

In some embodiments, a modified ITR can for example, comprise removal or deletion of all of a particular arm, e.g., all or part of the A-A′ arm, or all or part of the B-B′ arm or all or part of the C-C′ arm, or alternatively, the removal of 1, 2, 3, 4, 5, 6, 7, 8, 9 or more base pairs forming the stem of the loop so long as the final loop capping the stem (e.g., single arm) is still present (e.g., see ITR-21 in FIG. 7A of PCT/US2018/064242, filed Dec. 6, 2018). In some embodiments, a modified ITR can comprise the removal of 1, 2, 3, 4, 5, 6, 7, 8, 9 or more base pairs from the B-B′ arm. In some embodiments, a modified ITR can comprise the removal of 1, 2, 3, 4, 5, 6, 7, 8, 9 or more base pairs from the C-C′ arm (see, e.g., ITR-1 in FIG. 3B, or ITR-45 in FIG. 7A of PCT/US2018/064242, filed Dec. 6, 2018). In some embodiments, a modified ITR can comprise the removal of 1, 2, 3, 4, 5, 6, 7, 8, 9 or more base pairs from the C-C′ arm and the removal of 1, 2, 3, 4, 5, 6, 7, 8, 9 or more base pairs from the B-B′ arm. Any combination of removal of base pairs is envisioned, for example, 6 base pairs can be removed in the C-C′ arm and 2 base pairs in the B-B′ arm. As an illustrative example, FIG. 3B shows an exemplary modified ITR with at least 7 base pairs deleted from each of the C portion and the C′ portion, a substitution of a nucleotide in the loop between C and C′ region, and at least one base pair deletion from each of the B region and B′ regions such that the modified ITR comprises two arms where at least one arm (e.g., C-C′) is truncated. In some embodiments, the modified ITR also comprises at least one base pair deletion from each of the B region and B′ regions, such that the B-B′ arm is also truncated relative to WT ITR.

In some embodiments, a modified ITR can have between 1 and 50 (e.g. 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50) nucleotide deletions relative to a full-length wild-type ITR sequence. In some embodiments, a modified ITR can have between 1 and 30 nucleotide deletions relative to a full-length WT ITR sequence. In some embodiments, a modified ITR has between 2 and 20 nucleotide deletions relative to a full-length wild-type ITR sequence.

In some embodiments, a modified ITR does not contain any nucleotide deletions in the RBE-containing portion of the A or A′ regions, so as not to interfere with DNA replication (e.g. binding to an RBE by Rep protein, or nicking at a terminal resolution site). In some embodiments, a modified ITR encompassed for use herein has one or more deletions in the B, B′, C, and/or C region as described herein.

In some embodiments, a ceDNA vector for insertion of a transgene at a GSH locus as disclosed herein, comprising a symmetric ITR pair or asymmetric ITR pair, also can comprise one or more regulatory switch as disclosed herein and at least one modified ITR selected having the nucleotide sequence selected from any of the group consisting of: SEQ ID NO: 3, 4, 15-47, 101-116 or 165-187.

In another embodiment, the structure of the structural element can be modified. For example, the structural element a change in the height of the stem and/or the number of nucleotides in the loop. For example, the height of the stem can be about 2, 3, 4, 5, 6, 7, 8, or 9 nucleotides or more or any range therein. In one embodiment, the stem height can be about 5 nucleotides to about 9 nucleotides and functionally interacts with Rep. In another embodiment, the stem height can be about 7 nucleotides and functionally interacts with Rep. In another example, the loop can have 3, 4, 5, 6, 7, 8, 9, or 10 nucleotides or more or any range therein.

In another embodiment, the number of GAGY binding sites or GAGY-related binding sites within the RBE or extended RBE can be increased or decreased. In one example, the RBE or extended RBE, can comprise 1, 2, 3, 4, 5, or 6 or more GAGY binding sites or any range therein. Each GAGY binding site can independently be an exact GAGY sequence or a sequence similar to GAGY as long as the sequence is sufficient to bind a Rep protein.

In another embodiment, the spacing between two elements (such as but not limited to the RBE and a hairpin) can be altered (e.g., increased or decreased) to alter functional interaction with a large Rep protein. For example, the spacing can be about 1, 2, 3, 4, 5, 6, 7, 8, 9, 10, 11, 12, 13, 14, 15, 16, 17, 18, 19, 20, or 21 nucleotides or more or any range therein.

The ceDNA vector described herein can include an ITR structure that is modified with respect to the wild type AAV2 ITR structure disclosed herein, but still retains an operable RBE, trs and RBE′ portion. FIG. 2A and FIG. 2B show one possible mechanism for the operation of a trs site within a wild type ITR structure portion of a ceDNA vector. In some embodiments, the ceDNA vector contains one or more functional ITR polynucleotide sequences that comprise a Rep-binding site (RBS; 5′-GCGCGCTCGCTCGCTC-3′ (SEQ ID NO: 60) for AAV2) and a terminal resolution site (TRS; 5′-AGTT (SEQ ID NO: 62)). In some embodiments, at least one ITR (wt or modified ITR) is functional. In alternative embodiments, where a ceDNA vector comprises two modified ITRs that are different or asymmetrical to each other, at least one modified ITR is functional and at least one modified ITR is non-functional.

In some embodiments, the modified ITR (e.g., the left or right ITR) of a ceDNA vector for insertion of a transgene at a GSH locus as described herein has modifications within the loop arm, the truncated arm, or the spacer. Exemplary sequences of ITRs having modifications within the loop arm, the truncated arm, or the spacer are listed in Table 2 (i.e., SEQ ID NOS: 135-190, 200-233); Table 3 (e.g., SEQ ID Nos: 234-263); Table 4 (e.g., SEQ ID NOs: 264-293); Table 5 (e.g., SEQ ID Nos: 294-318); Table 6 (e.g., SEQ ID NO: 319-468; and Tables 7-9 (e.g., SEQ ID Nos: 101-110, 111-112, 115-134) or Table 10A or 10B (e.g., SEQ ID Nos: 9, 100, 469-483, 484-499) of International application PCT/US18/49996, which is incorporated herein in its entirety by reference.

In some embodiments, the modified ITR for use in a ceDNA vector for insertion of a transgene into a GSH comprising an asymmetric ITR pair, or symmetric mod-ITR pair is selected from any or a combination of those shown in Tables 2, 3, 4, 5, 6, 7, 8, 9 and 10A-10B of International application PCT/US18/49996 which is incorporated herein in its entirety by reference.

Additional exemplary modified ITRs for use in a ceDNA vector for insertion of a transgene into a GSH that comprises an asymmetric ITR pair, or symmetric mod-ITR pair in each of the above classes are provided in Tables 8A and 8B. The predicted secondary structure of the Right modified ITRs in Table 4A are shown in FIG. 7A of International Application PCT/US2018/064242, filed Dec. 6, 2018, and the predicted secondary structure of the Left modified ITRs in Table 4B are shown in FIG. 7B of International Application PCT/US2018/064242, filed Dec. 6, 2018, which is incorporated herein in its entirety by reference.

Table 8A and Table 8B show exemplary right and left modified ITRs.

Table 8A: Exemplary modified right ITRs. These exemplary modified right ITRs can comprise the RBE of GCGCGCTCGCTCGCTC-3′ (SEQ ID NO: 60), spacer of ACTGAGGC (SEQ ID NO: 69), the spacer complement GCCTCAGT (SEQ ID NO: 70) and RBE′ (i.e., complement to RBE) of GAGCGAGCGAGCGCGC (SEQ ID NO: 71).

TABLE 8A
Exemplary Right modified ITRs

| ITR | | SEQ ID |
|---|---|---|
| Construct | Sequence | NO: |
| ITR-18 | AGGAACCCCTAGTGATGGAGTTGGCCACTCCCTCTCTGCGCGCTCG | 15 |
| Right | CTCGCTCACTGAGGCGCACGCCCGGGTTTCCCGGGCGGCCTCAGTG | |
| | AGCGAGCGAGCGCGCAGCTGCCTGCAGG | |
| ITR-19 | AGGAACCCCTAGTGATGGAGTTGGCCACTCCCTCTCTGCGCGCTCG | 16 |
| Right | CTCGCTCACTGAGGCCGACGCCCGGGCTTTGCCCGGGCGGCCTCA | |
| | GTGAGCGAGCGAGCGCGCAGCTGCCTGCAGG | |
| ITR-20 | AGGAACCCCTAGTGATGGAGTTGGCCACTCCCTCTCTGCGCGCTCG | 17 |
| Right | CTCGCTCACTGAGGCCGGGCGACCAAAGGTCGCCCGACGCCCGGG | |
| | CGCCTCAGTGAGCGAGCGAGCGCGCAGCTGCCTGCAGG | |
| ITR-21 | AGGAACCCCTAGTGATGGAGTTGGCCACTCCCTCTCTGCGCGCTCG | 18 |
| Right | CTCGCTCACTGAGGCTTTGCCTCAGTGAGCGAGCGAGCGCGCAGC | |
| | TGCCTGCAGG | |
| ITR-22 | AGGAACCCCTAGTGATGGAGTTGGCCACTCCCTCTCTGCGCGCTCG | |
| Right | CTCGCTCACTGAGGCCGGGCGACAAAGTCGCCCGACGCCCGGGCT | 19 |
| | TTGCCCGGGCGGCCTCAGTGAGCGAGCGAGCGCGCAGCTGCCTGC | |
| | AGG | |
| ITR-23 | AGGAACCCCTAGTGATGGAGTTGGCCACTCCCTCTCTGCGCGCTCG | 20 |
| Right | CTCGCTCACTGAGGCCGGGCGAAAATCGCCCGACGCCCGGGCTTT | |
| | GCCCGGGCGGCCTCAGTGAGCGAGCGAGCGCGCAGCTGCCTGCAG | |
| | G | |
| ITR-24 | AGGAACCCCTAGTGATGGAGTTGGCCACTCCCTCTCTGCGCGCTCG | 21 |
| Right | CTCGCTCACTGAGGCCGGGCGAAACGCCCGACGCCCGGGCTTTGC | |
| | CCGGGCGGCCTCAGTGAGCGAGCGAGCGCGCAGCTGCCTGCAGG | |
| ITR-25 | AGGAACCCCTAGTGATGGAGTTGGCCACTCCCTCTCTGCGCGCTCG | 22 |
| Right | CTCGCTCACTGAGGCCGGGCAAAGCCCGACGCCCGGGCTTTGCCC | |
| | GGGCGGCCTCAGTGAGCGAGCGAGCGCGCAGCTGCCTGCAGG | |
| ITR-26 | AGGAACCCCTAGTGATGGAGTTGGCCACTCCCTCTCTGCGCGCTCG | 23 |
| Right | CTCGCTCACTGAGGCCGGGCGACCAAAGGTCGCCCGACGCCCGGG | |
| | TTTCCCGGGCGGCCTCAGTGAGCGAGCGAGCGCGCAGCTGCCTGC | |
| | AGG | |
| ITR-27 | AGGAACCCCTAGTGATGGAGTTGGCCACTCCCTCTCTGCGCGCTCG | 24 |
| Right | CTCGCTCACTGAGGCCGGGCGACCAAAGGTCGCCCGACGCCCGGT | |
| | TTCCGGGCGGCCTCAGTGAGCGAGCGAGCGCGCAGCTGCCTGCAG | |
| | G | |
| ITR-28 | AGGAACCCCTAGTGATGGAGTTGGCCACTCCCTCTCTGCGCGCTCG | 25 |
| Right | CTCGCTCACTGAGGCCGGGCGACCAAAGGTCGCCCGACGCCCGTT | |
| | TCGGGCGGCCTCAGTGAGCGAGCGAGCGCGCAGCTGCCTGCAGG | |
| ITR-29 | AGGAACCCCTAGTGATGGAGTTGGCCACTCCCTCTCTGCGCGCTCG | 26 |
| Right | CTCGCTCACTGAGGCCGGGCGACCAAAGGTCGCCCGACGCCCTTT | |
| | GGGCGGCCTCAGTGAGCGAGCGAGCGCGCAGCTGCCTGCAGG | |
| ITR-30 | AGGAACCCCTAGTGATGGAGTTGGCCACTCCCTCTCTGCGCGCTCG | 27 |
| Right | CTCGCTCACTGAGGCCGGGCGACCAAAGGTCGCCCGACGCCTTTG | |
| | GCGGCCTCAGTGAGCGAGCGAGCGCGCAGCTGCCTGCAGG | |
| ITR-31 | AGGAACCCCTAGTGATGGAGTTGGCCACTCCCTCTCTGCGCGCTCG | 28 |
| Right | CTCGCTCACTGAGGCCGGGCGACCAAAGGTCGCCCGACGCTTTGC | |
| | GGCCTCAGTGAGCGAGCGAGCGCGCAGCTGCCTGCAGG | |
| ITR-32 | AGGAACCCCTAGTGATGGAGTTGGCCACTCCCTCTCTGCGCGCTCG | 29 |
| Right | CTCGCTCACTGAGGCCGGGCGACCAAAGGTCGCCCGACGTTTCGG | |
| | CCTCAGTGAGCGAGCGAGCGCGCAGCTGCCTGCAGG | |
| ITR-49 | AGGAACCCCTAGTGATGGAGTTGGCCACTCCCTCTCTGCGCGCTCG | 30 |
| Right | CTCGCTCACTGAGGCCGGGCGACCAAAGGTCGCCCGACGGCCTCA | |
| | GTGAGCGAGCGAGCGCGCAGCTGCCTGCAGG | |
| ITR-50 | AGGAACCCCTAGTGATGGAGTTGGCCACTCCCTCTCTGCGCGCTCG | 31 |
| right | CTCGCTCACTGAGGCCGGGCGACCAAAGGTCGCCCGACGCCCGGG | |
| | CGGCCTCAGTGAGCGAGCGAGCGCGCAGCTGCCTGCAGG | |

TABLE 8B
Exemplary modified left ITRs

| | | |
|---|---|---|
| ITR-33 | CCTGCAGGCAGCTGCGCGCTCGCTCGCTCACTGAGGCCGCCCGGG | 32 |
| Left | AAACCCGGGCGTGCGCCTCAGTGAGCGAGCGAGCGCGCAGAGAG | |
| | GGAGTGGCCAACTCCATCACTAGGGGTTCCT | |
| ITR-34 | CCTGCAGGCAGCTGCGCGCTCGCTCGCTCACTGAGGCCGTCGGGC | 33 |
| Left | GACCTTTGGTCGCCCGGCCTCAGTGAGCGAGCGAGCGCGCAGAGA | |
| | GGGAGTGGCCAACTCCATCACTAGGGGTTCCT | |
| ITR-35 | CCTGCAGGCAGCTGCGCGCTCGCTCGCTCACTGAGGCCGCCCGGG | 34 |
| Left | CAAAGCCCGGGCGTCGGCCTCAGTGAGCGAGCGAGCGCGCAGAG | |
| | AGGGAGTGGCCAACTCCATCACTAGGGGTTCCT | |
| ITR-36 | CCTGCAGGCAGCTGCGCGCTCGCTCGCTCACTGAGGCGCCCGGGC | 35 |
| Left | GTCGGGCGACCTTTGGTCGCCCGGCCTCAGTGAGCGAGCGAGCGC | |
| | GCAGAGAGGGAGTGGCCAACTCCATCACTAGGGGTTCCT | |
| ITR-37 | CCTGCAGGCAGCTGCGCGCTCGCTCGCTCACTGAGGCAAAGCCTC | 36 |
| Left | AGTGAGCGAGCGAGCGCGCAGAGAGGGAGTGGCCAACTCCATCA | |
| | CTAGGGGTTCCT | |
| ITR-38 | CCTGCAGGCAGCTGCGCGCTCGCTCGCTCACTGAGGCCGCCCGGG | 37 |
| Left | CAAAGCCCGGGCGTCGGGCGACTTTGTCGCCCGGCCTCAGTGAGC | |
| | GAGCGAGCGCGCAGAGAGGGAGTGGCCAACTCCATCACTAGGGGT | |
| | TCCT | |
| ITR-39 | CCTGCAGGCAGCTGCGCGCTCGCTCGCTCACTGAGGCCGCCCGGG | 38 |
| Left | CAAAGCCCGGGCGTCGGGCGATTTTCGCCCGGCCTCAGTGAGCGA | |
| | GCGAGCGCGCAGAGAGGGAGTGGCCAACTCCATCACTAGGGGTTC | |
| | CT | |
| ITR-40 | CCTGCAGGCAGCTGCGCGCTCGCTCGCTCACTGAGGCCGCCCGGG | 39 |
| Left | CAAAGCCCGGGCGTCGGGCGTTTCGCCCGGCCTCAGTGAGCGAGC | |
| | GAGCGCGCAGAGAGGGAGTGGCCAACTCCATCACTAGGGGTTCCT | |
| ITR-41 | CCTGCAGGCAGCTGCGCGCTCGCTCGCTCACTGAGGCCGCCCGGG | 40 |
| Left | CAAAGCCCGGGCGTCGGGCTTTGCCCGGCCTCAGTGAGCGAGCGA | |
| | GCGCGCAGAGAGGGAGTGGCCAACTCCATCACTAGGGGTTCCT | |
| ITR-42 | CCTGCAGGCAGCTGCGCGCTCGCTCGCTCACTGAGGCCGCCCGGG | 41 |
| Left | AAACCCGGGCGTCGGGCGACCTTTGGTCGCCCGGCCTCAGTGAGC | |
| | GAGCGAGCGCGCAGAGAGGGAGTGGCCAACTCCATCACTAGGGGT | |
| | TCCT | |
| ITR-43 | CCTGCAGGCAGCTGCGCGCTCGCTCGCTCACTGAGGCCGCCCGGA | 42 |
| Left | AACCGGGCGTCGGGCGACCTTTGGTCGCCCGGCCTCAGTGAGCGA | |
| | GCGAGCGCGCAGAGAGGGAGTGGCCAACTCCATCACTAGGGGTTC | |
| | CT | |
| ITR-44 | CCTGCAGGCAGCTGCGCGCTCGCTCGCTCACTGAGGCCGCCCGAA | 43 |
| Left | ACGGGCGTCGGGCGACCTTTGGTCGCCCGGCCTCAGTGAGCGAGC | |
| | GAGCGCGCAGAGAGGGAGTGGCCAACTCCATCACTAGGGGTTCCT | |
| ITR-45 | CCTGCAGGCAGCTGCGCGCTCGCTCGCTCACTGAGGCCGCCCAAA | 44 |
| Left | GGGCGTCGGGCGACCTTTGGTCGCCCGGCCTCAGTGAGCGAGCGA | |
| | GCGCGCAGAGAGGGAGTGGCCAACTCCATCACTAGGGGTTCCT | |
| ITR-46 | CCTGCAGGCAGCTGCGCGCTCGCTCGCTCACTGAGGCCGCCAAAG | 45 |
| Left | GCGTCGGGCGACCTTTGGTCGCCCGGCCTCAGTGAGCGAGCGAGC | |
| | GCGCAGAGAGGGAGTGGCCAACTCCATCACTAGGGGTTCCT | |
| ITR-47 | CCTGCAGGCAGCTGCGCGCTCGCTCGCTCACTGAGGCCGCAAAGC | 46 |
| Left | GTCGGGCGACCTTTGGTCGCCCGGCCTCAGTGAGCGAGCGAGCGC | |
| | GCAGAGAGGGAGTGGCCAACTCCATCACTAGGGGTTCCT | |
| ITR-48 | CCTGCAGGCAGCTGCGCGCTCGCTCGCTCACTGAGGCCGAAACGT | 47 |
| Left | CGGGCGACCTTTGGTCGCCCGGCCTCAGTGAGCGAGCGAGCGCGC | |
| | AGAGAGGGAGTGGCCAACTCCATCACTAGGGGTTCCT | |

TABLE 8B Exemplary modified left ITRs.
These exemplary modified left ITRs can comprise the RBE of GCGCGCTCGCTCGCTC-3′ (SEQ ID NO: 60), spacer of ACTGAGGC (SEQ ID NO: 69), the spacer complement GCCTCAGT (SEQ ID NO: 70) and RBE complement (RBE′) of GAGCGAGCGAGCGCGC (SEQ ID NO: 71).

In one embodiment, a ceDNA vector for insertion of a transgene into a GSH comprises, in the 5′ to 3′ direction: a first adeno-associated virus (AAV) inverted terminal repeat (ITR), a HA-L, a nucleotide sequence of interest (for example an expression cassette as described herein), a HA-R and a second AAV ITR, where the first ITR (5′ ITR) and the second ITR (3′ ITR) are asymmetric with respect to each other—that is, they have a different 3D-spatial configuration from one another. As an exemplary embodiment, the first ITR can be a wild-type ITR and the second ITR can be a mutated or modified ITR, or vice versa, where the first ITR can be a mutated or modified ITR and the second ITR a wild-type ITR. In some embodiment, the first ITR and the second ITR are both mod-ITRs, but have different sequences, or have different modifications, and thus are not the same modified ITRs, and have different 3D spatial configurations. Stated differently, a ceDNA vector for insertion of a transgene into a GSH with asymmetric ITRs comprises ITRs where any changes in one ITR relative to the WT-ITR are not reflected in the other ITR; or alternatively, where the asymmetric ITRs have a the modified asymmetric ITR pair can have a different sequence and different three-dimensional shape with respect to each other. Exemplary asymmetric ITRs in the ceDNA vector and for use to generate a ceDNA-plasmid are shown in Table 8A and 8B.

In an alternative embodiment, a ceDNA vector for insertion of a transgene into a GSH comprises two symmetrical mod-ITRs—that is, both ITRs have the same sequence, but are reverse complements (inverted) of each other. In some embodiments, a symmetrical mod-ITR pair comprises at least one or any combination of a deletion, insertion, or substitution relative to wild type ITR sequence from the same AAV serotype. The additions, deletions, or substitutions in the symmetrical ITR are the same but the reverse complement of each other. For example, an insertion of 3 nucleotides in the C region of the 5′ ITR would be reflected in the insertion of 3 reverse complement nucleotides in the corresponding section in the C′ region of the 3′ ITR. Solely for illustration purposes only, if the addition is AACG in the 5′ ITR, the addition is CGTT in the 3′ ITR at the corresponding site. For example, if the 5′ ITR sense strand is ATCGATCG with an addition of AACG between the G and A to result in the sequence ATCGAACGATCG (SEQ ID NO: 51). The corresponding 3′ ITR sense strand is CGATCGAT (the reverse complement of ATCGATCG) with an addition of CGTT (i.e. the reverse complement of AACG) between the T and C to result in the sequence CGATCGTTCGAT (SEQ ID NO: 49) (the reverse complement of ATCGAACGATCG) (SEQ ID NO: 51).

In alternative embodiments, the modified ITR pair are substantially symmetrical as defined herein—that is, the modified ITR pair can have a different sequence but have corresponding or the same symmetrical three-dimensional shape. For example, one modified ITR can be from one serotype and the other modified ITR be from a different serotype, but they have the same mutation (e.g., nucleotide insertion, deletion or substitution) in the same region. Stated differently, for illustrative purposes only, a 5′ mod-ITR can be from AAV2 and have a deletion in the C region, and the 3′ mod-ITR can be from AAV5 and have the corresponding deletion in the C′ region, and provided the 5′ mod-ITR and the 3′ mod-ITR have the same or symmetrical three-dimensional spatial organization, they are encompassed for use herein as a modified ITR pair.

In some embodiments, a substantially symmetrical mod-ITR pair has the same A, C-C′ and B-B′ loops in 3D space, e.g., if a modified ITR in a substantially symmetrical mod-ITR pair has a deletion of a C-C′ arm, then the cognate mod-ITR has the corresponding deletion of the C-C′ loop and also has a similar 3D structure of the remaining A and B-B′ loops in the same shape in geometric space of its cognate mod-ITR. By way of example only, substantially symmetrical ITRs can have a symmetrical spatial organization such that their structure is the same shape in geometrical space. This can occur, e.g., when a G-C pair is modified, for example, to a C-G pair or vice versa, or A-T pair is modified to a T-A pair, or vice versa. Therefore, using the exemplary example above of modified 5′ ITR as a ATCGAACGATCG (SEQ ID NO: 51), and modified 3′ ITR as CGATCGTTCGAT (SEQ ID NO: 49) (i.e., the reverse complement of ATCGAACGATCG (SEQ ID NO: 51)), these modified ITRs would still be symmetrical if, for example, the 5′ ITR had the sequence of ATCGAACCATCG (SEQ ID NO: 50), where G in the addition is modified to C, and the substantially symmetrical 3′ ITR has the sequence of CGATCGTTCGAT (SEQ ID NO: 49), without the corresponding modification of the T in the addition to a. In some embodiments, such a modified ITR pair are substantially symmetrical as the modified ITR pair has symmetrical stereochemistry.

Table 9 shows exemplary symmetric modified ITR pairs (i.e. a left modified ITRs and the symmetric right modified ITR). The bold (red) portion of the sequences identify partial ITR sequences (i.e., sequences of A-A′, C-C′ and B-B′ loops), also shown in FIGS. 31A-46B of International Application PCT/US2018/064242, filed Dec. 6, 2018, which is incorporated herein in its entirety. These exemplary modified ITRs can comprise the RBE of GCGCGCTCGCTCGCTC-3′ (SEQ ID NO: 60), spacer of ACTGAGGC (SEQ ID NO: 69), the spacer complement GCCTCAGT (SEQ ID NO: 70) and RBE′ (i.e., complement to RBE) of GAGCGAGCGAGCGCGC (SEQ ID NO: 71).

TABLE 9
exemplary symmetric modified ITR pairs

| LEFT modified ITR | | Symmetric RIGHT modified ITR | |
|---|---|---|---|
| (modified 5′ ITR) | | (modified 3′ ITR) | |
| SEQ ID | CCTGCAGGCAGCTGCGCGCTC | SEQ ID NO: 15 | AGGAACCCCTAGTGATG |
| NO: 32 | GCTCGCTCACTGAGGCCGCC | (ITR-18, right) | GAGTTGGCCACTCCCTCT |
| (ITR-33 | CGGGAAACCCGGGCGTGCGC | | CTGCGCGCTCGCTCGC |
| left) | CTCAGTGAGCGAGCGAGCGC | | TCACTGAGGCGCACGC |
| | GCAGAGAGGGAGTGGCCAACT | | CCGGGTTTCCCGGGCG |
| | CCATCACTAGGGGTTCCT | | GCCTCAGTGAGCGAGC |
| | | | GAGCGCGCAGCTGCCT |
| | | | GCAGG |
| SEQ ID | CCTGCAGGCAGCTGCGCGCTC | SEQ ID NO: 48 | AGGAACCCCTAGTGATG |
| NO: 33 | GCTCGCTCACTGAGGCCGTC | (ITR-51, right) | GAGTTGGCCACTCCCTCT |
| (ITR-34 | GGGCGACCTTTGGTCGCCCG | | CTGCGCGCTCGCTCGC |
| left) | GCCTCAGTGAGCGAGCGAGC | | TCACTGAGGCCGGGCG |
| | GCGCAGAGAGGGAGTGGCCA | | ACCAAAGGTCGCCCGA |
| | ACTCCATCACTAGGGGTTCCT | | CGGCCTCAGTGAGCGA |
| | | | GCGAGCGCGCAGCTGC |
| | | | CTGCAGG |
| SEQ ID | CCTGCAGGCAGCTGCGCGCTC | SEQ ID NO: 16 | AGGAACCCCTAGTGATG |
| NO: 34 | GCTCGCTCACTGAGGCCGCC | (ITR-19, right) | GAGTTGGCCACTCCCTCT |
| (ITR-35 | CGGGCAAAGCCCGGGCGTCG | | CTGCGCGCTCGCTCGC |
| left) | GCCTCAGTGAGCGAGCGAGC | | TCACTGAGGCCGACGC |
| | GCGCAGAGAGGGAGTGGCCA | | CCGGGCTTTGCCCGGG |
| | ACTCCATCACTAGGGGTTCCT | | CGGCCTCAGTGAGCGA |
| | | | GCGAGCGCGCAGCTGC |
| | | | CTGCAGG |
| SEQ ID | CCTGCAGGCAGCTGCGCGCTC | SEQ ID NO: 17 | AGGAACCCCTAGTGATG |
| NO: 35 | GCTCGCTCACTGAGGCGCCC | (ITR-20, right) | GAGTTGGCCACTCCCTCT |
| (ITR-36 | GGGCGTCGGGCGACCTTTGG | | CTGCGCGCTCGCTCGC |
| left) | TCGCCCGGCCTCAGTGAGCG | | TCACTGAGGCCGGGCG |
| | AGCGAGCGCGCAGAGAGGGA | | ACCAAAGGTCGCCCGA |
| | GTGGCCAACTCCATCACTAGG | | CGCCCGGGCGCCTCAG |
| | GGTTCCT | | TGAGCGAGCGAGCGCG |
| | | | CAGCTGCCTGCAGG |
| SEQ ID | CCTGCAGGCAGCTGCGCGCTC | SEQ ID NO: 18 | AGGAACCCCTAGTGATG |
| NO: 36 | GCTCGCTCACTGAGGCAAAG | (ITR-21, right) | GAGTTGGCCACTCCCTCT |
| (ITR-37 | CCTCAGTGAGCGAGCGAGCG | | CTGCGCGCTCGCTCGC |
| left) | CGCAGAGAGGGAGTGGCCAAC | | TCACTGAGGCTTTGCC |
| | TCCATCACTAGGGGTTCCT | | TCAGTGAGCGAGCGAG |
| | | | CGCGCAGCTGCCTGCAG |
| | | | G |
| SEQ ID | CCTGCAGGCAGCTGCGCGCTC | SEQ ID NO: 19 | AGGAACCCCTAGTGATG |
| NO: 37 | GCTCGCTCACTGAGGCCGCC | (ITR-22 right) | GAGTTGGCCACTCCCTCT |
| (ITR-38 | CGGGCAAAGCCCGGGCGTCG | | CTGCGCGCTCGCTCGC |
| left) | GGCGACTTTGTCGCCCGGCC | | TCACTGAGGCCGGGCG |
| | TCAGTGAGCGAGCGAGCGCG | | ACAAAGTCGCCCGACG |
| | CAGAGAGGGAGTGGCCAACTC | | CCCGGGCTTTGCCCGG |
| | CATCACTAGGGGTTCCT | | GCGGCCTCAGTGAGCG |
| | | | AGCGAGCGCGCAGCTG |
| | | | CCTGCAGG |
| SEQ ID | CCTGCAGGCAGCTGCGCGCTC | SEQ ID NO: 20 | AGGAACCCCTAGTGATG |
| NO: 38 | GCTCGCTCACTGAGGCCGCC | (ITR-23, right) | GAGTTGGCCACTCCCTCT |
| (ITR-39 | CGGGCAAAGCCCGGGCGTCG | | CTGCGCGCTCGCTCGC |
| left) | GGCGATTTTCGCCCGGCCTC | | TCACTGAGGCCGGGCG |
| | AGTGAGCGAGCGAGCGCGCA | | AAAATCGCCCGACGCC |
| | GAGAGGGAGTGGCCAACTCCA | | CGGGCTTTGCCCGGGC |
| | TCACTAGGGGTTCCT | | GGCCTCAGTGAGCGAG |
| | | | CGAGCGCGCAGCTGCC |
| | | | TGCAGG |
| SEQ ID | CCTGCAGGCAGCTGCGCGCTC | SEQ ID NO: 21 | AGGAACCCCTAGTGATG |
| NO: 39 | GCTCGCTCACTGAGGCCGCC | (ITR-24, right) | GAGTTGGCCACTCCCTCT |
| (ITR-40 | CGGGCAAAGCCCGGGCGTCG | | CTGCGCGCTCGCTCGC |
| left) | GGCGTTTCGCCCGGCCTCAG | | TCACTGAGGCCGGGCG |
| | TGAGCGAGCGAGCGCGCAGA | | AAACGCCCGACGCCCG |
| | GAGGGAGTGGCCAACTCCATC | | GGCTTTGCCCGGGCGG |
| | ACTAGGGGTTCCT | | CCTCAGTGAGCGAGCG |
| | | | AGCGCGCAGCTGCCTGC |
| | | | AGG |
| SEQ ID | CCTGCAGGCAGCTGCGCGCTC | SEQ ID NO: 22 | AGGAACCCCTAGTGATG |
| NO: 40 | GCTCGCTCACTGAGGCCGCC | (ITR-25 right) | GAGTTGGCCACTCCCTCT |
| (ITR-41 | CGGGCAAAGCCCGGGCGTCG | | CTGCGCGCTCGCTCGC |
| left) | GGCTTTGCCCGGCCTCAGTG | | TCACTGAGGCCGGGCA |
| | AGCGAGCGAGCGCGCAGAGA | | AAGCCCGACGCCCGGG |
| | GGGAGTGGCCAACTCCATCAC | | CTTTGCCCGGGCGGCC |
| | TAGGGGTTCCT | | TCAGTGAGCGAGCGAG |
| | | | CGCGCAGCTGCCTGCAG |
| | | | G |
| SEQ ID | CCTGCAGGCAGCTGCGCGCTC | SEQ ID NO: 23 | AGGAACCCCTAGTGATG |
| NO: 41 | GCTCGCTCACTGAGGCCGCC | (ITR-26 right) | GAGTTGGCCACTCCCTCT |
| (ITR-42 | CGGGAAACCCGGGCGTCGGG | | CTGCGCGCTCGCTCGC |
| left) | CGACCTTTGGTCGCCCGGCC | | TCACTGAGGCCGGGCG |
| | TCAGTGAGCGAGCGAGCGCG | | ACCAAAGGTCGCCCGA |
| | CAGAGAGGGAGTGGCCAACTC | | CGCCCGGGTTTCCCGG |
| | CATCACTAGGGGTTCCT | | GCGGCCTCAGTGAGCG |
| | | | AGCGAGCGCGCAGCTG |
| | | | CCTGCAGG |
| SEQ ID | CCTGCAGGCAGCTGCGCGCTC | SEQ ID NO: 24 | AGGAACCCCTAGTGATG |
| NO: | GCTCGCTCACTGAGGCCGCC | (ITR-27 right) | GAGTTGGCCACTCCCTCT |
| 42(ITR-43 | CGGAAACCGGGCGTCGGGCG | | CTGCGCGCTCGCTCGC |
| left) | ACCTTTGGTCGCCCGGCCTC | | TCACTGAGGCCGGGCG |
| | AGTGAGCGAGCGAGCGCGCA | | ACCAAAGGTCGCCCGA |
| | GAGAGGGAGTGGCCAACTCCA | | CGCCCGGTTTCCGGGC |
| | TCACTAGGGGTTCCT | | GGCCTCAGTGAGCGAG |
| | | | CGAGCGCGCAGCTGCC |
| | | | TGCAGG |
| SEQ ID | CCTGCAGGCAGCTGCGCGCTC | SEQ ID NO: 25 | AGGAACCCCTAGTGATG |
| NO: 43 | GCTCGCTCACTGAGGCCGCC | (ITR-28 right) | GAGTTGGCCACTCCCTCT |
| (ITR-44 | CGAAACGGGCGTCGGGCGAC | | CTGCGCGCTCGCTCGC |
| left) | CTTTGGTCGCCCGGCCTCAG | | TCACTGAGGCCGGGCG |
| | TGAGCGAGCGAGCGCGCAGA | | ACCAAAGGTCGCCCGA |
| | GAGGGAGTGGCCAACTCCATC | | CGCCCGTTTCGGGCGG |
| | ACTAGGGGTTCCT | | CCTCAGTGAGCGAGCG |
| | | | AGCGCGCAGCTGCCTGC |
| | | | AGG |
| SEQ ID | CCTGCAGGCAGCTGCGCGCTC | SEQ ID NO: 26 | AGGAACCCCTAGTGATG |
| NO: 44 | GCTCGCTCACTGAGGCCGCC | (ITR-29, right) | GAGTTGGCCACTCCCTCT |
| (ITR-45 | CAAAGGGCGTCGGGCGACCT | | CTGCGCGCTCGCTCGC |
| left) | TTGGTCGCCCGGCCTCAGTG | | TCACTGAGGCCGGGCG |
| | AGCGAGCGAGCGCGCAGAGA | | ACCAAAGGTCGCCCGA |
| | GGGAGTGGCCAACTCCATCAC | | CGCCCTTTGGGCGGCC |
| | TAGGGGTTCCT | | TCAGTGAGCGAGCGAG |
| | | | CGCGCAGCTGCCTGCAG |
| | | | G |
| SEQ ID | CCTGCAGGCAGCTGCGCGCTC | SEQ ID NO: 27 | AGGAACCCCTAGTGATG |
| NO: 45 | GCTCGCTCACTGAGGCCGCC | (ITR-30, right) | GAGTTGGCCACTCCCTCT |
| (ITR-46 | AAAGGCGTCGGGCGACCTTT | | CTGCGCGCTCGCTCGC |
| left) | GGTCGCCCGGCCTCAGTGAG | | TCACTGAGGCCGGGCG |
| | CGAGCGAGCGCGCAGAGAGG | | ACCAAAGGTCGCCCGA |
| | GAGTGGCCAACTCCATCACTA | | CGCCTTTGGCGGCCTC |
| | GGGGTTCCT | | AGTGAGCGAGCGAGCG |
| | | | CGCAGCTGCCTGCAGG |
| SEQ ID | CCTGCAGGCAGCTGCGCGCTC | SEQ ID NO: 28 | AGGAACCCCTAGTGATG |
| NO: 46 | GCTCGCTCACTGAGGCCGCA | (ITR-31, right) | GAGTTGGCCACTCCCTCT |
| (ITR-47, | AAGCGTCGGGCGACCTTTGG | | CTGCGCGCTCGCTCGC |
| left) | TCGCCCGGCCTCAGTGAGCG | | TCACTGAGGCCGGGCG |
| | AGCGAGCGCGCAGAGAGGGA | | ACCAAAGGTCGCCCGA |
| | GTGGCCAACTCCATCACTAGG | | CGCTTTGCGGCCTCAG |
| | GGTTCCT | | TGAGCGAGCGAGCGCG |
| | | | CAGCTGCCTGCAGG |
| SEQ ID | CCTGCAGGCAGCTGCGCGCTC | SEQ ID NO: 29 | AGGAACCCCTAGTGATG |
| NO: 47 | GCTCGCTCACTGAGGCCGAA | (ITR-32 right) | GAGTTGGCCACTCCCTCT |
| (ITR-48, | ACGTCGGGCGACCTTTGGTC | | CTGCGCGCTCGCTCGC |
| left) | GCCCGGCCTCAGTGAGCGAG | | TCACTGAGGCCGGGCG |
| | CGAGCGCGCAGAGAGGGAGT | | ACCAAAGGTCGCCCGA |
| | GGCCAACTCCATCACTAGGGG | | CGTTTCGGCCTCAGTG |
| | TTCCT | | AGCGAGCGAGCGCGCA |
| | | | GCTGCCTGCAGG |

In some embodiments, a ceDNA vector for insertion of a transgene into a GSH comprising an asymmetric ITR pair can comprise an ITR with a modification corresponding to any of the modifications in ITR sequences or ITR partial sequences shown in any one or more of Tables 8A-8B herein, or the sequences shown in FIG. 7A-7B of International Application PCT/US2018/064242, filed Dec. 6, 2018, which is incorporated herein in its entirety, or disclosed in Tables 2, 3, 4, 5, 6, 7, 8, 9 or 10A-10B of International application PCT/US18/49996 filed Sep. 7, 2018 which is incorporated herein in its entirety by reference.

V. Exemplary ceDNA Vectors for Insertion of a Transgene at a GSH Locus

As described above, the present disclosure relates to recombinant ceDNA expression vectors and ceDNA vectors for insertion of a transgene at a GSH locus as disclosed herein, where the ceDNA vector comprises any one of: an asymmetrical ITR pair, a symmetrical ITR pair, or substantially symmetrical ITR pair as described above, that flank a HA-L and HA-R, and located between the HA-L and HA-R is a transgene to be inserted into the genome of a host cell. In certain embodiments, the disclosure relates to recombinant ceDNA vectors for insertion of a transgene at a GSH locus, the ceDNA vector having ITR sequences flanking GSH specific HA-L and HA-R regions, where located between the HA-L and HA-R is one or more transgenes, where the ITR sequences are asymmetrical, symmetrical or substantially symmetrical relative to each other as defined herein, and the ceDNA further comprises a nucleotide sequence of interest (for example an expression cassette comprising the nucleic acid of a transgene) located between the flanking ITRs, wherein said nucleic acid molecule is devoid of viral capsid protein coding sequences.

The ceDNA vector for insertion of a transgene at a GSH locus may be any ceDNA vector that can be conveniently subjected to recombinant DNA procedures including nucleotide sequence(s) as described herein, provided at least one ITR is altered. The ceDNA vectors of the present disclosure are compatible with the host cell into which the ceDNA vector is to be introduced. In certain embodiments, the ceDNA vectors may be linear. In certain embodiments, the ceDNA vectors may exist as an extrachromosomal entity. In certain embodiments, the ceDNA vectors of the present disclosure may contain an element(s) that permits integration of a donor sequence into the host cell's genome. As used herein “transgene” and “heterologous nucleotide sequence” are synonymous.

Referring now to FIG. 1A, shows an exemplary ceDNA vector for insertion of a transgene into the genome of a host cells at a specific GSH locus. FIGS. 1B-1H show schematics of the functional components of two non-limiting plasmids useful in making the ceDNA vectors of the present disclosure are shown. FIG. 1B, 1C, 1D, 1G show the construct of ceDNA vectors or the corresponding sequences of ceDNA plasmids. ceDNA vectors are capsid-free and can be obtained from a plasmid encoding in this order: a first ITR, an expressible transgene cassette and a second ITR, where the first and second ITR sequences are asymmetrical, symmetrical or substantially symmetrical relative to each other as defined herein. ceDNA vectors are capsid-free and can be obtained from a plasmid encoding in this order: a first ITR, a HA-L, an expressible transgene (protein or nucleic acid), a HA-R and a second ITR, where the first and second ITR sequences are asymmetrical, symmetrical or substantially symmetrical relative to each other as defined herein. In some embodiments, the expressible transgene cassette includes, as needed: an enhancer/promoter, one or more homology arms, a donor sequence, a post-transcription regulatory element (e.g., WPRE, e.g., SEQ ID NO: 67)), and a polyadenylation and termination signal (e.g., BGH polyA, e.g., SEQ ID NO: 68).

Such exemplary ceDNA vectors shown in FIGS. 1A-1H can be administered with one or more gene editing molecules, such as those including an RNA guided nuclease, the components required for gene editing may include a nuclease, a guide RNA (if Cas9 or the like is utilized), a donor sequence. Such embodiments increase the efficiency of gene editing compared to approaches that require distinct or various particles to deliver the gene editing components.

In alternative embodiments, in addition to a ceDNA vector comprising ITRs flanking a HA-L and HA-R, which in turn flank the transgene to be inserted, the ceDNA vector can further include a “gene editing cassette” between the ITRs, but outside the homology arms. Exemplary “all-in-one” ceDNA vector for insertion of a gene into a GSH locus are shown in FIGS. 8, 9D and 10. Such all-in one ceDNA vectors for insertion of a transgene into a GSH locus can comprise at least one of the following: a nuclease, a guide RNA, an activator RNA, and a control element. Suitable ceDNA vectors in accordance with the present disclosure may be obtained by following the Examples below. In certain embodiments, the disclosure relates to a ceDNA vector comprising two ITRs, a gene editing cassette comprising at least two components of a gene editing system, e.g. CAS and at least one gRNA, or two ZNFs, etc., and a transgene flanked by a HA-L and HA-R that are specific to a GSH locus shown in Table 1A or 1B, Thus, in some embodiments, the ceDNA vectors comprise two ITRs, a transgene flanked by HA-L and HA-R, and multiple components of a gene editing system, including a gene editing molecule of interest (e.g., a nuclease (e.g., sequence specific nuclease), one or more guide RNA, Cas or other ribonucleoprotein (RNP), or any combination thereof. In some embodiments, a nuclease can be inactivated/diminished after gene editing, reducing or eliminating off-target editing, if any, that would otherwise occur with the persistence of an added nuclease within cells.

In another aspect, the present disclosure relates to kits including one or more ceDNA vectors for use in any one of the methods described herein. The methods and compositions described herein also provide for gene editing systems comprising a cellular switch, for example, as described by Oakes et al. Nat. Biotechnol. 34:646-651 (2016), the contents of which are herein incorporated by reference in their entirety.

FIG. 5 is a gel confirming the production of ceDNA from multiple plasmid constructs using the method described in the Examples. The ceDNA is confirmed by a characteristic band pattern in the gel, as discussed with respect to FIG. 4A above and in the Examples.

Referring now to FIG. 7, a nonlimiting exemplary ceDNA vector in accordance with the present disclosure is shown including a first and second ITR, where the ITR sequences are asymmetrical, symmetrical or substantially symmetrical relative to each other as defined herein, a first nucleotide sequence including a 5′ homology arm (HA-L), a transgene sequence, and a 3′ homology arm (HA-R). Non-limiting examples of the nucleic acid constructs of the present disclosure include a nucleic acid construct including a wild-type functioning ITR of AAV2 having the nucleotide sequence of SEQ ID NO:1, or SEQ ID NO:2 and further an altered ITR of AAV2 having at least 60%, more preferably at least 65%, more preferably at least 70%, more preferably at least 75%, more preferably at least 80%, more preferably at least 85%, even more preferably at least 90%, and most preferably at least 95% sequence identity to the nucleotide sequence of SEQ ID NO: 3 or SEQ ID NO: 4. Additional ITRs are described in International Patent applications PCT/US18/49996 and PCT/US18/14122, each herein incorporated by reference in their entirety.

In another embodiment, a ceDNA vector for insertion of a transgene into a GSH locus as disclosed herein encodes a nuclease and one or more guide RNAs that are directed to each of the ceDNA ITRs, or directed to HA-L or HA-R homology arms, for torsional release and more efficient homology directed repair (HDR). The nuclease need not be a mutant nuclease, e.g. the donor HDR template may be released from ceDNA by such cleavage.

In some embodiments, in one nonlimiting example, a ceDNA vector for insertion of a transgene into a GSH locus as disclosed herein comprise a 5′ and 3′ homology arm to a PAX5 or other gene listed in in Table 1 or 1B. When the ceDNA vector is cleaved with the one or more restriction endonucleases specific for the restriction site(s), the resulting expression cassette comprises the 5′ homology arm-donor sequence-3′ homology arm, and can be more readily recombined with the desired GSH genomic locus. In certain aspects, the ceDNA vector itself may encode the restriction endonuclease such that upon delivery of the ceDNA vector to the nucleus, the restriction endonuclease is expressed and able to cleave the ceDNA vector. In certain aspects, the restriction endonuclease or one or more gene editing molecules are encoded on a second ceDNA vector which is separately delivered. In certain aspects, the restriction endonuclease is introduced to the nucleus by a non-ceDNA-based means of delivery. Accordingly, in some embodiments, the technology described herein enables more than one ceDNA being delivered to a subject. As discussed herein, in one embodiment, a ceDNA can have the homology arms (HA-L and HA-R) flanking a transgene where the HA-L and HA-R targets a specific GSH locus.

### A. Homology-Arms (HA)

In some embodiments, ceDNA vector for insertion of a transgene at a GSH locus as disclosed herein, where the ceDNA vector comprises a transgene flanked by a HA-L and a HA-R, and also comprises a gene editing cassette, the transgene is inserted into the genome with homologous recombination. It is contemplated herein that a homology directed repair template can be used to insert a new sequence, for example, to manufacture a therapeutic protein. In some embodiments, the HA-L and HA-R are designed to serve as a template in homologous recombination, such as within or near a target GSH locus nicked or cleaved by a nuclease described herein, e.g., an RNA-guided endonuclease, such as a CRISPR enzyme as a part of a CRISPR complex, or ZFN or TALEN. Each homology arm polynucleotide can be of any suitable length, such as about or more than about 10, 15, 20, 25, 50, 75, 100, 150, 200, 500, 1000, or more nucleotides in length. In some embodiments, each homology arm polynucleotide is complementary to a portion of a polynucleotide comprising a GSH locus in the host cell genome. When optimally aligned, a HA-L and HA-R polynucleotide can overlap with one or more nucleotides of the GSH locus (e.g., about or more than about 1, 5, 10, 15, 20, 25, 30, 35, 40, 45, 50, 60, 70, 80, 90, 100 or more nucleotides). In some embodiments, when the polynucleotide of one or both homology arms and the GSH locus are optimally aligned, homology recombination can occur. In one embodiment, the homology arms are directional (i.e., not identical and therefore bind to the sequence in a particular orientation).

In some embodiments, the homology arms are substantially identical to a portion of a GSH locus disclosed in Table 1A or 1B and can comprises at least one nucleotide change. As will be readily appreciated by one of skill in the art insertion of the transgene flanked by the HA-L and HA-R can result in a change in an exon sequence, an intron sequence, a regulatory sequence, a transcriptional control sequence, a translational control sequence, a splicing site, or a non-coding sequence of the gene at the GSH locus.

In certain embodiments, a ceDNA vector for insertion of a transgene into the GSH locus of the genome of a host cell comprises two ITRs that flank a 5′ homology arm, and/or a 3′ homology arm. At a minimum in certain such embodiments, ceDNA comprises, from 5′ to 3′, a 5′ GSH HDR arm (i.e., HA-L), a transgene, a 3′ HDR arm (i.e., HA-R), wherein the at least one ITR is upstream of the 5′ HDR arm and the other ITR is downstream of the 3′ HDR arm. In certain embodiments, the transgene is a nucleotide sequence to be inserted into a GSH locus of a host cell. In certain embodiments, the transgene (also referred to as donor sequence) is not originally present in the host cell or may be foreign to the host cell. In certain embodiments, the transgene is an endogenous sequence present at a site other than the predetermined target site. In certain embodiments, the transgene is an endogenous sequence similar to that of the pre-determined target site (e.g., replaces an existing erroneous sequence). In certain embodiments, the transgene is a sequence endogenous to the host cell, but which is present at a site other than the predetermined target site. In some embodiments, the transgene is a coding sequence or non-coding sequence. In some embodiments, the transgene is a mutant locus of a gene. In certain embodiments, the transgene may be an exogenous gene to be inserted into the chromosome, a modified sequence that replaces the endogenous sequence at the target site, a regulatory element, a tag or a coding sequence encoding a reporter protein and/or RNA. In some embodiments, the transgene may be inserted in frame into the coding sequence of a target gene for expression of a fusion protein. In certain embodiments, the transgene is inserted in-frame behind an endogenous promoter such that the transgene is regulated similarly to the naturally-occurring sequence.

In certain embodiments, the transgene may optionally include a promoter therein as described above in order to drive a coding sequence. Such embodiments may further include a poly-A tail within the transgene to facilitate expression.

In certain embodiments, the donor sequence or transgene may be a predetermined size, or sized by one of ordinary skill in the art. In certain embodiments, the transgene may be at least or about any of 10 base pairs, 15 base pairs, 20 base pairs, 25 base pairs, 50 base pairs, 60 base pairs, 75 base pairs, 100 base pairs, at least 150 base pairs, 200 base pairs, 300 base pairs, 500 base pairs, 800 base pairs, 1000 base pairs, 1,500 base pairs, 2,000 base pairs, 2500 base pairs, 3000 base pairs, 4000 base pairs, 4500 base pairs, and 5,000 base pairs in length or about 1 base pair to about 10 base pairs, or about 10 base pairs to about 50 base pairs, or between about 50 base pairs to about 100 base pairs, or between about 100 base pairs to about 500 base pairs, or between about 500 base pairs to about 5,000 base pairs in length.

Non-limiting examples of suitable transgene(s) for use in accordance with the present disclosure include a promoter-less coding sequence corresponding to one or more disease-related sequences having at least 60%, more preferably at least 65%, more preferably at least 70%, more preferably at least 75%, more preferably at least 80%, more preferably at least 85%, even more preferably at least 90%, and most preferably at least 95% sequence identity to one of the disease-related molecules described herein. In one embodiment, the coding sequence has at least 60%, more preferably at least 65%, more preferably at least 70%, more preferably at least 75%, more preferably at least 80%, more preferably at least 85%, even more preferably at least 90%, and most preferably at least 95% sequence identity to the naturally occurring transgene. In certain embodiments, such as where the sequence is added rather than replaced, a promoter can be provided.

For integration of the transgene into the host cell genome, the ceDNA vector may rely on the polynucleotide sequence encoding the transgene or any other element of the vector for integration into the genome by homologous recombination such as the 5′ and 3′ homology arms shown therein (see e.g., FIG. 7). For example, the ceDNA vector may contain nucleotides encoding 5′ and 3′ GSH-specific homology arms for directing integration by homologous recombination into the genome of the host cell at a precise location(s) in the chromosome(s). To increase the likelihood of integration at a precise GSH locus, each of the 5′ and 3′ homology arms may include a sufficient number of nucleic acids, such as 50 to 5,000 base pairs, or 100 to 5,000 base pairs, or 500 to 5,000 base pairs, which have a high degree of sequence identity or homology to the corresponding GSH target sequence to enhance the probability of homologous recombination. The 5′ and 3′ homology arms may be any sequence that is homologous with the target sequence in the genome of the host cell. Furthermore, the 5′ and 3′ homology arms may be non-encoding or encoding nucleotide sequences. In certain embodiments, the homology between the 5′ homology arm and the corresponding sequence on the chromosome is at least any of 80%, 85%, 90%, 95%, 97%, 98%, 99%, or 100%. In certain embodiments, the homology between the 3′ homology arm and the corresponding sequence on the chromosome is at least any of 80%, 85%, 90%, 95%, 97%, 98%, 99%, or 100%. In certain embodiments, the 5′ and/or 3′ homology arms can be homologous to a sequence immediately upstream and/or downstream of the integration or DNA cleavage site on the chromosome. Alternatively, the 5′ and/or 3′ homology arms can be homologous to a sequence that is distant from the integration or DNA cleavage site, such as at least 1, 2, 5, 10, 15, 20, 25, 30, 50, 100, 200, 300, 400, or 500 bp away from the integration or DNA cleavage site, or partially or completely overlapping with the DNA cleavage site. In certain embodiments, the 3′ homology arm of the nucleotide sequence is proximal to the altered ITR.

In certain embodiments, the efficiency of integration of the transgene is improved by extraction of the cassette comprising the transgene (e.g., the transgene flanked by the GSH-homology arms) from the ceDNA vector prior to integration. In one nonlimiting example, a specific restriction site may be engineered 5′ to the 5′ homology arm, or 3′ to the 3′ homology arm, or both. If such a restriction site is present with respect to both homology arms, then the restriction site may be the same or different between the two homology arms. When the ceDNA vector is cleaved with the one or more restriction endonucleases specific for the engineered restriction site(s), the resulting cassette comprises the 5′ homology arm-transgene-3′ homology arm, and can be more readily recombined with the desired genomic locus. It will be appreciated by one of ordinary skill in the art that this cleaved cassette may additionally comprise other elements such as, but not limited to, one or more of the following: a regulatory region, a nuclease, and an additional transgene. In certain aspects, the ceDNA vector itself may encode the restriction endonuclease such that upon delivery of the ceDNA vector to the nucleus the restriction endonuclease is expressed and able to cleave the vector. In certain aspects, the restriction endonuclease is encoded on a second ceDNA vector which is separately delivered. In certain aspects, the restriction endonuclease is introduced to the nucleus by a non-ceDNA-based means of delivery. In certain embodiments, the restriction endonuclease is introduced after the ceDNA vector is delivered to the nucleus. In certain embodiments, the restriction endonuclease and the ceDNA vector are transported to the nucleus simultaneously. In certain embodiments, the restriction endonuclease is already present upon introduction of the ceDNA vector.

In certain embodiments, the transgene is foreign to the 5′ homology arm or 3′ homology arm. In certain embodiments, the transgene is not endogenously found between the sequences comprising the 5′ homology arm and 3′ homology arm. In certain embodiments, the transgene is not endogenous to the native sequence comprising the 5′ homology arm or the 3′ homology arm. In certain embodiments, the 5′ homology arm is homologous to a nucleotide sequence upstream of a nuclease cleavage site on a chromosome. In certain embodiments, the 3′ homology arm is homologous to a nucleotide sequence downstream of a nuclease cleavage site on a chromosome. In certain embodiments, the 5′ homology arm or the 3′ homology arm are proximal to the at least one altered ITR. In certain embodiments, the 5′ homology arm or the 3′ homology arm are about 250 to 2000 bp.

Non-limiting examples of suitable 5′ homology arms for use in accordance with the present disclosure include a 5′ homology arm (HA-L) specific to the PAX5 GSH locus, having at least 60%, more preferably at least 65%, more preferably at least 70%, more preferably at least 75%, more preferably at least 80%, more preferably at least 85%, even more preferably at least 90%, and most preferably at least 95% sequence identity to a suitable segment of between 200-800 nucleotides within the nucleic acid of Accession number NC_000009.12 (PAX5 gene) or a 5′ homology arm (HA-L) specific to the PAX5 GSH locus, consisting of a suitable segment that has homology to at least 200-800 nucleotides within the nucleic acid of Accession number NC_000009.12 (PAX5 gene). Such segments can be all of the respective sequences.

Non-limiting examples of suitable 3′ homology arms for use in accordance with the present disclosure include a 3′ homology arm (HA-R) specific to the PAX5 GSH locus, having at least 60%, more preferably at least 65%, more preferably at least 70%, more preferably at least 75%, more preferably at least 80%, more preferably at least 85%, even more preferably at least 90%, and most preferably at least 95% sequence identity to a suitable segment of between 200-800 nucleotides within the nucleic acid of Accession number NC_000009.12 (PAX5 gene) or a 3′ homology arm (HA-R) specific to the PAX5 GSH locus, consisting of a suitable segment that has homology to at least 200-800 nucleotides within the nucleic acid of Accession number NC_000009.12 (PAX5 gene). Such segments can be all of the respective sequences.

Non-limiting examples of suitable 5′ homology arms for use in accordance with the present disclosure include a 5′ homology arm (HA-L) specific to the KIF6 GSH locus, having at least 60%, more preferably at least 65%, more preferably at least 70%, more preferably at least 75%, more preferably at least 80%, more preferably at least 85%, even more preferably at least 90%, and most preferably at least 95% sequence identity to a suitable segment of between 200-800 nucleotides within the region of Chromosome 6: 39,329,990-39,725,405 (Kif6 gene) or a 5′ homology arm (HA-L) specific to the PAX5 GSH locus, consisting of a suitable segment that has homology to at least 200-800 nucleotides within the nucleic acid within the region of Chromosome 6: 39,329,990-39,725,405 (Kif6 gene). Such segments can be all of the respective sequences.

Non-limiting examples of suitable 3′ homology arms for use in accordance with the present disclosure include a 3′ homology arm (HA-R) specific to the KIF6 GSH locus, having at least 60%, more preferably at least 65%, more preferably at least 70%, more preferably at least 75%, more preferably at least 80%, more preferably at least 85%, even more preferably at least 90%, and most preferably at least 95% sequence identity to a suitable segment of between 200-800 nucleotides within the nucleic acid of within the region of Chromosome 6: 39,329,990-39,725,405 (Kif6 gene) or a 3′ homology arm (HA-R) specific to the KIF5 GSH locus, consisting of a suitable segment that has homology to at least 200-800 nucleotides within the nucleic acid within the region of Chromosome 6: 39,329,990-39,725,405 (Kif6 gene). Such segments can be all of the respective sequences.

In one embodiment, a ceDNA vector for insertion of a transgene into a GSH loci comprising a transgene flanked between a GSH-specific HA-L and GSH specific HA-R, as described herein, can be administered in conjunction with another vector (e.g., an additional ceDNA vector, a lentiviral vector, a viral vector, or a plasmid) that encodes a Cas nickase (nCas; e.g., Cas9 nickase). It is contemplated herein that such an nCas enzyme is used in conjunction with a guide RNA that comprises homology to HA-L in a ceDNA vector as described herein and can be used, for example, to release physically constrained sequences or to provide torsional release. Releasing physically constrained sequences can, for example, “unwind” the ceDNA vector such that a homology directed repair (HDR) template homology arm(s) within the ceDNA vector are exposed for interaction with the genomic sequence. In addition, it is contemplated herein that such a system can be used to deactivate ceDNA vectors, if necessary. It will be understood by one of skill in the art that a Cas enzyme that induces a double-stranded break in the ceDNA vector would be a stronger deactivator of such ceDNA vectors. In one embodiment, the guide RNA comprises homology to a sequence inserted into the ceDNA vector such as a sequence encoding a nuclease or the donor sequence or template. In another embodiment, the guide RNA comprises homology to an inverted terminal repeat (ITR) or the homology/insertion elements of the ceDNA vector. In some embodiments, a ceDNA vector as described herein comprises an ITR on each of the 5′ and 3′ ends, thus a guide RNA with homology to the ITRs will produce nicking of the one or more ITRs substantially equally. In some embodiments, a guide RNA has homology to some portion of the ceDNA vector and the donor sequence or template (e.g., to assist with unwinding the ceDNA vector). It is also contemplated herein that there are certain sites on the ceDNA vectors that when nicked may result in the inability of the ceDNA vector to be retained in the nucleus. One of ordinary skill in the art can readily identify such sequences and can thus avoid engineering guide RNAs to such sequence regions. Alternatively, modifying the subcellular localization of a ceDNA vector to a region outside the nuclease by using a guide RNA that nicks sequences responsible for nuclear localization can be used as a method of deactivating the ceDNA vector, if necessary or desired.

In certain embodiments, other integration strategies and components are suitable for use in accordance with ceDNA vectors of the present disclosure. For example, although not shown in FIGS. 1A-1H or FIG. 7-10, in one embodiment, a ceDNA vector in accordance with the present disclosure may include an expression cassette flanked by ribosomal DNA (rDNA) sequences capable of homologous recombination into genomic rDNA. Similar strategies have been performed, for example, in Lisowski, et al., Ribosomal DNA Integrating rAAV-rDNA Vectors Allow for Stable Transgene Expression, The American Society of Gene and Cell Therapy, 18 Sep. 2012 (herein incorporated by reference in its entirety) where rAAV-rDNA vectors were demonstrated. In certain embodiments, delivery of ceDNA-rDNA vectors may integrate into the genomic rDNA locus with increased frequency, where the integrations are specific to the rDNA locus. Moreover, a ceDNA-rDNA vector containing a human factor IX (hFIX) or human Factor VIII expression cassette increases therapeutic levels of serum hFIX or human Factor VIII. Because of the relative safety of integration in the rDNA locus, ceDNA-rDNA vectors expand the usage of ceDNA for therapeutics requiring long-term gene transfer into dividing cells.

In one embodiment, a promoterless ceDNA vector is contemplated for delivery of a homology repair template (e.g., a repair sequence with two flanking homology arms) but does not comprise nucleic acid sequences encoding a nuclease or guide RNA.

The methods and compositions described herein can be used in methods comprising homology recombination, for example, as described in Rouet et al. Proc Natl Acad Sci 91:6064-6068 (1994); Chu et al. Nat Biotechnol 33:543-548 (2015); Richardson et al. Nat Biotechnol 33:339-344 (2016); Komor et al. Nature 533:420-424 (2016); the contents of each of which are incorporated by reference herein in their entirety.

The methods and compositions described herein can be used in methods comprising homology recombination, for example, as described in Rouet et al. Proc Natl Acad Sci 91:6064-6068 (1994); Chu et al. Nat Biotechnol 33:543-548 (2015); Richardson et al. Nat Biotechnol 33:339-344 (2016); Komor et al. Nature 533:420-424 (2016); the contents of each of which are incorporated by reference herein in their entirety.

### B. Gene Editing Cassette Components

### (i) Nucleases and DNA Endonucleases

As discussed herein, in addition to the transgene flanked by GSH specific 5′ HA and a GSH specific 3′ HA, the ceDNA vector can comprise a gene editing cassette that is located 5′ of the HA-L, but flanked by the ITRs (see, e.g., FIG. 8 and FIG. 9D). The gene editing cassette can comprise one or more of: a sgRNA expression unit and/or a nuclease expressing unit, where the nuclease expressing unit comprises one or more gene editing molecule, an enhancer (Enh), a promoter (pro), an intron (e.g., synthetic or natural occurring intron with splice donor and acceptor seq), nuclear localization signal (NLS) upstream of a nuclease (e.g., nucleic acid with an ORF encoding a Cas9, ZFN, Talen, or other endonuclease sequences). The sgRNA expression unit can comprise a promoter, e.g., U6 promoter which drives the expression of at least 1, or at least 2, or at least 3 or at least 4 or more sgRNAs. Transport of the nuclease to the nuclei can be increased or improved by using a nuclear localization signal (NLS) fused into the 5′ or 3′ nuclease protein (e.g., the nuclease expressing unit, such as Cas9, ZFN, TALEN etc.). Each of the components of the gene editing cassette are discussed herein.

In some embodiments, the ceDNA vector for insertion of a transgene into a GSH loci as disclosed herein can also include one or more guide RNAs (e.g., sgRNA) for targeting the cutting of the genomic DNA, as described herein. In some embodiments, the ceDNA vector can further comprise a nuclease enzyme and activator RNA, as described herein for the actual gene editing steps. Alternatively, the nuclease enzyme and activator RNA can be provided separately in a different ceDNA vector, or by a non-ceDNA vector means.

A ceDNA vector for insertion of a transgene into a GSH locus as disclosed herein may contain a nucleotide sequence that encodes a nuclease, such as a sequence-specific nuclease. Sequence-specific or site-specific nucleases can be used to introduce site-specific double strand breaks or nicks at targeted genomic loci. This nucleotide cleavage, e.g., DNA or RNA cleavage, stimulates the natural repair machinery, e.g., DNA repair machinery, leading to one of two possible repair pathways. In the absence of a donor template, the break will be repaired by non-homologous end joining (NHEJ), an error-prone repair pathway that leads to small insertions or deletions of DNA (see e.g., Suzuki et al. Nature 540:144-149 (2016), the contents of which are incorporated by reference in its entirety). This method can be used to intentionally disrupt, delete, or alter the reading frame of targeted gene sequences. However, if a donor template is provided in addition to the nuclease, then the cellular machinery will repair the break by homologous recombination (HDR), which is enhanced several orders of magnitude in the presence of DNA cleavage, or by insertion of the donor template via NHEJ.

The methods can be used to introduce specific changes in the DNA sequence at target sites. The term “site-specific nuclease” as used herein refers to an enzyme capable of specifically recognizing and cleaving a particular DNA sequence. The site-specific nuclease may be engineered. Examples of engineered site-specific nucleases include zinc finger nucleases (ZFNs), TAL effector nucleases (TALENs), meganucleases, and CRISPR/Cas9-enzymes and engineered derivatives. As will be appreciated by those of skill in the art, the endonucleases necessary for gene editing can be expressed transiently, as there is generally no further need for the endonuclease once gene editing is complete. Such transient expression can reduce the potential for off-target effects and immunogenicity. Transient expression can be accomplished by any known means in the art, and may be conveniently effected using a regulatory switch as described herein.

In some embodiments, the nucleotide sequence encoding the nuclease is cDNA. Non-limiting examples of sequence-specific nucleases include RNA-guided nuclease, zinc finger nuclease (ZFN), a transcription activator-like effector nuclease (TALEN) or a meganuclease. Non-limiting examples of suitable RNA-guided nucleases include CRISPR enzymes as described herein.

The nucleases described herein can be altered, e.g., engineered to design sequence specific nuclease (see e.g., U.S. Pat. No. 8,021,867). Nucleases can be designed using the methods described in e.g., Certo, M T et al. Nature Methods (2012) 9:073-975; U.S. Pat. Nos. 8,304,222; 8,021,867; 8,119,381; 8,124,369; 8,129,134; 8,133,697; 8,143,015; 8,143,016; 8,148,098; or 8,163,514, the contents of each are incorporated herein by reference in their entirety. Alternatively, nuclease with site specific cutting characteristics can be obtained using commercially available technologies e.g., Precision BioSciences' Directed Nuclease Editor™ genome editing technology.

In certain embodiments, for example when using a promoterless ceDNA construct comprising a homology directed repair template, the guide RNA and/or Cas enzyme, or any other nuclease, are delivered in trans, e.g. by administering i) a nucleic acid encoding a guide RNA, ii) or an mRNA encoding a the desired nuclease, e.g. Cas enzyme, or other nuclease iii) or by administering a ribonucleotide protein (RNP) complex comprising a Cas enzyme and a guide RNA, or iv) e.g., delivery of recombinant nuclease proteins by vector, e.g. viral, plasmid, or another ceDNA vector. In certain aspects, the molecules delivered in trans are delivered by means of one or more additional ceDNA vectors which can be co-administered or administered sequentially to the first ceDNA vector.

Accordingly, in one embodiment, a ceDNA vector for insertion of a transgene into a GSH locus as disclosed herein can comprise an endonuclease (e.g., Cas9) that is transcriptionally regulated by an inducible promoter. In some embodiments, the endonuclease is on a separate ceDNA vector, which can be administered to a subject with a ceDNA comprising homology arms and a donor sequence, which can optionally also comprise guide RNA (sgRNAs). In alternative embodiments, the endonuclease can be on an all-in-one ceDNA vector as described herein.

In some embodiments, a ceDNA vector for insertion of a transgene into a GSH locus as disclosed herein that encodes an endonuclease as described herein can be under control of a promoter. Non-limiting examples of inducible promoters include chemically-regulated promoters, which regulate transcriptional activity by the presence or absence of, for example, alcohols, tetracycline, steroids, metal, and pathogenesis-related proteins (e.g., salicylic acid, ethylene, and benzothiadiazole), and physically-regulated promoters, which regulate transcriptional activity by, for example, the presence or absence of light and low or high temperatures. Modulation of the inducible promoter allows for the turning off or on of gene-editing activity of a ceDNA vector. Inducible Cas9 promoters are further reviewed, for example in Cao J., et al. Nucleic Acids Research. 44(19)2016, and Liu K I, et al. Nature Chemical Biol. 12: 90-987 (2016), which are incorporated herein in their entireties.

In one embodiment, a ceDNA vector for insertion of a transgene into a GSH locus as disclosed herein as described herein further comprises a second endonuclease that temporally targets and inhibits the activity of the first endonuclease (e.g., Cas9). Endonucleases that target and inhibit the activity of other endonucleases can be determined by those skilled in the art. In another embodiment, the ceDNA vector described herein further comprises temporal expression of an “anti-CRISPR gene” (e.g., L. monocytogenes ArcIIa). As used herein, “anti-CRISPR gene” refers to a gene shown to inhibit the commonly used S. pyogenes Cas9. In another embodiment, the second endonuclease that targets and inhibits the activity of the first endonuclease activity, or the anti-CRISPR gene, is comprised in a second ceDNA vector that is administered after the desired gene-editing is complete. Alternatively, the second endonuclease targets and inhibits a gene of interest, for example, a gene that has been transcriptionally enhanced by a ceDNA vector as described herein.

A ceDNA vector for insertion of a transgene into a GSH locus as disclosed herein as described herein, can include a nucleotide sequence encoding a transcriptional activator that activates a target gene. For example, the transcriptional activator may be engineered. For example, an engineered transcriptional activator may be a CRISPR/Cas9-based system, a zinc finger fusion protein, or a TALE fusion protein. The CRISPR/Cas9-based system, as described above, may be used to activate transcription of a target gene with RNA. The CRISPR/Cas9-based system may include a fusion protein, as described above, wherein the second polypeptide domain has transcription activation activity or histone modification activity. For example, the second polypeptide domain may include VP64 or p300. Alternatively, the transcriptional activator may be a zinc finger fusion protein. The zinc finger targeted DNA-binding domains, as described above, can be combined with a domain that has transcription activation activity or histone modification activity. For example, the domain may include VP64 or p300. TALE fusion proteins may be used to activate transcription of a target gene. The TALE fusion protein may include a TALE DNA-binding domain and a domain that has transcription activation activity or histone modification activity. For example, the domain may include VP64 or p300.

Another method for modulating gene expression at the transcription level is by targeting epigenetic modifications using modified DNA endonucleases as described herein. Modulation of gene expression at the epigenetic level has the advantage of being inherited by daughter cells at a higher rate than the activation/inhibition achieved using CRISPRa or CRISPRi. In one embodiment, dCas9 fused to a catalytic domain of p300 acetyltransferase can be used with the methods and compositions described herein to make epigenetic modifications (e.g., increase histone modification) to a desired region of the genome. Epigenetic modifications can also be achieved using modified TALEN constructs, such as a fusion of a TALEN to the Teti demethylase catalytic domain (see e.g., Maeder et al. Nature Biotechnology 31(12):1137-42 (2013)) or a TAL effector fused to LSD1 histone demethylase (Mendenhall et al. Nature Biotechnology 31(12):1133-6 (2013)).

### (ii) Modified DNA Endonucleases, Nuclease-Dead Cas9 and Uses Thereof

Unlike viral vectors, the ceDNA vectors as described herein do not have a capsid that limits the size or number of nucleic acid sequences, effector sequences, regulatory sequences etc. that can be delivered to a cell. Accordingly, a ceDNA vector for insertion of a transgene into a GSH locus, comprising a HA-L transgene HA-R, as disclosed herein can also comprise nucleic acids encoding nuclease-dead DNA endonucleases, nickases, or other DNA endonucleases with modified function (e.g., unique PAM binding sequence) for enhanced production of a desired vector and/or delivery of the vector to a cell. Such ceDNA vectors can also include promoter sequences and other regulatory or effector sequences as desired. Given the lack of size constraint, one of skill in the art will readily understand that, for example, that expression of a desired nuclease with modified function, and optionally, at least one guide RNA can be from nucleic acid sequences on the same vector and can be under the control of the same or different promoters. It is also contemplated herein that at least two different modified endonucleases can be encoded in the same vector, for example, for multiplexed gene expression modulation (see “Multiplexed gene expression modulation” section herein) and under the control of the same or different promoters. Thus, one of skill in the art could combine the desired functionality of at least two different Cas9 endonucleases (e.g., at least 3, at least 4, at least 5, at least 6, at least 7, at least 8, at least 9, at least 10, or more) as desired including, for example, temporally regulated expression of at least two different modified endonucleases by one or more inducible promoters.

In some embodiments, a DNA endonuclease for use with the methods and compositions described herein, can be modified such that the DNA endonuclease retains DNA binding activity e.g., at a target site of the genome determined by a guide RNA sequence but does not retain cleavage activity (e.g., nuclease dead Cas9 (dCas9)) or has reduced cleavage activity (e.g., by at least 10%, at least 20%, at least 30%, at least 40%, at least 50%, at least 60%, at least 70%, at least 80%, at least 90%, at least 95%, at least 99%) as compared to the unmodified DNA endonuclease (e.g., Cas9 nickase). In some embodiments, a modified DNA endonuclease is used herein to inhibit expression of a target gene. For example, since a modified DNA endonuclease retains DNA binding activity, it can prevent the binding of RNA polymerase and/or displace RNA polymerase, which in turn prevents transcription of the target gene. Thus, expression of a gene product (e.g., mRNA, protein) from the desired gene is prevented.

For example, a “deactivated Cas9 (dCas9),” “nuclease dead Cas9” or an otherwise inactivated form of Cas9 can be introduced with a guide RNA that directs binding to a specific gene. Such binding can reduce in inhibition of expression of the target gene, if desired. In some embodiments, one may want to have the ability to reverse such gene expression inhibition. This can be achieved, for example, by providing different guide RNAs to the dead Cas9 protein to weaken the binding of Cas9 to the genomic site. Such reversal can occur in an iterative fashion where at least two or a series of guide RNAs designed to decrease the stability of the dead Cas9 binding are administered in succession. For example, each successive guide RNA can increase the instability from the degree of instability/stability of dead Cas9 binding produced by the guide RNA in the previous iteration. Thus, in some embodiments, one can use a dCas9 directed to a target gene sequence with a guide RNA to “inactivate a desired gene,” without cleavage of the genomic sequence, such that the gene of interest is not expressed in a functional protein form. In alternative embodiments, a guide RNA can be designed such that the stability of the dCas9 binding is reduced, but not eliminated. That is, the displacement of RNA polymerase is not complete thereby permitting the “reduction of gene expression” of the desired gene.

In certain embodiments, hybrid recombinases may be suitable for use in ceDNA vectors of the present disclosure to create integration cites on target DNA. For example, Hybrid recombinases based on activated catalytic domains derived from the resolvase/invertase family of serine recombinases fused to Cys2-His2 zinc-finger or TAL effector DNA-binding domains are a class of reagents capable improved targeting specificity in mammalian cells and achieve excellent rates of site-specific integration. Suitable hybrid recombinases encoded by nucleotides in ceDNA vectors in accordance with the present disclosure include those described in Gaj et al., Enhancing the Specificity of Recombinase-Mediated Genome Engineering through Dimer Interface Redesign, Journal of the American Chemical Society, Mar. 10, 2014 (herein incorporated by reference in its entirety).

(iii) Zinc Finger Endonucleases and TALENs

ZFNs and TALEN-based restriction endonuclease technology utilizes a non-specific DNA cutting enzyme which is linked to a specific DNA sequence recognizing peptide(s) such as zinc fingers and transcription activator-like effectors (TALEs). Typically, an endonuclease whose DNA recognition site and cleaving site are separate from each other is selected and its cleaving portion is separated and then linked to a sequence recognizing peptide, thereby yielding an endonuclease with very high specificity for a desired sequence. An exemplary restriction enzyme with such properties is FokI. Additionally, FokI has the advantage of requiring dimerization to have nuclease activity and this means the specificity increases dramatically as each nuclease partner recognizes a unique DNA sequence. To enhance this effect, FokI nucleases have been engineered that can only function as heterodimers and have increased catalytic activity. The heterodimer functioning nucleases avoid the possibility of unwanted homodimer activity and thus increase specificity of the double-stranded break.

Although the nuclease portions of both ZFNs and TALENs have similar properties, the difference between these engineered nucleases is in their DNA recognition peptide. ZFNs rely on Cys2-His2 zinc fingers and TALENs on TALEs. Both of these DNA recognizing peptide domains have the characteristic that they are naturally found in combination in their proteins. Cys2-His2 Zinc fingers typically happen in repeats that are 3 bp apart and are found in diverse combinations in a variety of nucleic acid interacting proteins such as transcription factors. TALEs on the other hand are found in repeats with a one-to-one recognition ratio between the amino acids and the recognized nucleotide pairs. Because both zinc fingers and TALEs happen in repeated patterns, different combinations can be tried to create a wide variety of sequence specificities. Approaches for making site-specific zinc finger endonucleases include, e.g., modular assembly (where Zinc fingers correlated with a triplet sequence are attached in a row to cover the required sequence), OPEN (low-stringency selection of peptide domains vs. triplet nucleotides followed by high-stringency selections of peptide combination vs. the final target in bacterial systems), and bacterial one-hybrid screening of zinc finger libraries, among others. ZFNs for use with the methods and compositions described herein can be obtained commercially from e.g., Sangamo Biosciences™ (Richmond, Calif.).

The terms “Transcription activator-like effector nucleases” or “TALENs” as used interchangeably herein refers to engineered fusion proteins of the catalytic domain of a nuclease, such as endonuclease FokI, and a designed TALE DNA-binding domain that may be targeted to a custom DNA sequence. A “TALEN monomer” refers to an engineered fusion protein with a catalytic nuclease domain and a designed TALE DNA-binding domain. Two TALEN monomers may be designed to target and cleave a TALEN target region.

The terms “Transcription activator-like effector” or “TALE” as used herein refers to a protein structure that recognizes and binds to a particular DNA sequence. The “TALE DNA-binding domain” refers to a DNA-binding domain that includes an array of tandem 33-35 amino acid repeats, also known as RVD modules, each of which specifically recognizes a single base pair of DNA. RVD modules can be arranged in any order to assemble an array that recognizes a defined sequence. A binding specificity of a TALE DNA-binding domain is determined by the RVD array followed by a single truncated repeat of 20 amino acids. A TALE DNA-binding domain may have 12 to 27 RVD modules, each of which contains an RVD and recognizes a single base pair of DNA. Specific RVDs have been identified that recognize each of the four possible DNA nucleotides (A, T, C, and G). Because the TALE DNA-binding domains are modular, repeats that recognize the four different DNA nucleotides may be linked together to recognize any particular DNA sequence. These targeted DNA-binding domains can then be combined with catalytic domains to create functional enzymes, including artificial transcription factors, methyltransferases, integrases, nucleases, and recombinases.

The TALENs may include a nuclease and a TALE DNA-binding domain that binds to the target sequence or gene in a TALEN target region. A “TALEN target region” includes the binding regions for two TALENs and the spacer region, which occurs between the binding regions. The two TALENs bind to different binding regions within the TALEN target region, after which the TALEN target region is cleaved. Examples of TALENs are described in International Patent Application WO2013163628, which is incorporated by reference in its entirety.

The terms “Zinc finger nuclease” or “ZFN” as used interchangeably herein refers to a chimeric protein molecule comprising at least one zinc finger DNA binding domain effectively linked to at least one nuclease or part of a nuclease capable of cleaving DNA when fully assembled. “Zinc finger” as used herein refers to a protein structure that recognizes and binds to DNA sequences. The zinc finger domain is the most common DNA-binding motif in the human proteome. A single zinc finger contains approximately 30 amino acids and the domain typically functions by binding 3 consecutive base pairs of DNA via interactions of a single amino acid side chain per base pair.

In certain embodiments, a ceDNA vector for insertion of a transgene into a GSH locus, comprising a HA-L transgene HA-R, as disclosed herein can comprise, outside of the HA region, nucleotide sequences encoding zinc-finger recombinases (ZFR) or chimeric proteins suitable for introducing targeted modifications into cells, such as mammalian cells. Unlike targeted nucleases and conventional SSR systems, ZFR specificity is the cooperative product of modular site-specific DNA recognition and sequence-dependent catalysis. ZFR's with diverse targeting capabilities can be generated with a plug-and-play manner. ZFR's including enhanced catalytic domains demonstrate improved targeting specificity and efficiency, and enable the site-specific delivery of therapeutic genes into the human genome with low toxicity. Mutagenesis of the Cre recombinase dimer interface also improves recombination specificity.

In embodiments, a ceDNA vector for insertion of a transgene into a GSH locus, comprising a HA-L transgene HA-R, as disclosed herein are suitable for use in nuclease free HDR systems such as those described in Porro et al., Promoterless gene targeting without nucleases rescues lethality of a Crigler-Najjar syndrome mouse model, EMBO Molecular Medicine, Jul. 27, 2017 (herein incorporated by reference in its entirety). In such embodiments, in vivo gene targeting approaches are suitable for ceDNA application based on the insertion of a donor sequence, without the use of nucleases. In some embodiments, the donor sequence may be promoterless.

While TALEN and ZFN are exemplified for use of the ceDNA vector for DNA editing (e.g., genomic DNA editing), also encompassed herein are use of mtZFN and mitoTALEN function, or mitochondrial-adapted CRISPR/Cas9 platform for use of the ceDNA vectors for editing of mitochondrial DNA (mtDNA), as described in Maeder, et al. “Genome-editing technologies for gene and cell therapy.” Molecular Therapy 24.3 (2016): 430-446 and Gammage P A, et al. Mitochondrial Genome Engineering: The Revolution May Not Be CRISPR-Ized. Trends Genet. 2018; 34(2):101-110.

Nucleic Acid-Guided Endonucleases

Different types of nucleic acid-guided endonucleases can be used in the compositions and methods of the invention to facilitate ceDNA-mediated gene editing. Exemplary, nonlimiting, types of nucleic acid-guided endonucleases suited for the compositions and methods of the invention include RNA-guided endonucleases, DNA-guided endonucleases, and single-base editors.

In some embodiments, the nuclease can be an RNA-guided endonuclease. As used herein, the term “RNA-guided endonuclease” refers to an endonuclease that forms a complex with an RNA molecule that comprises a region complementary to a selected target DNA sequence, such that the RNA molecule binds to the selected sequence to direct endonuclease activity to the selected target DNA sequence.

In one embodiment, the RNA-guided endonuclease is a CRISPR enzyme, as discussed herein. In some embodiments, the RNA-guided endonuclease comprises nickase activity. In some embodiments, the RNA-guided endonuclease directs cleavage of one or both strands at the location of a target sequence, such as within the target sequence and/or within the complement of the target sequence. In some embodiments, the RNA-guided endonuclease directs cleavage of one or both strands within about 1, 2, 3, 4, 5, 6, 7, 8, 9, 10, 15, 20, 25, 50, 100, 200, 500, or more base pairs from the first or last nucleotide of a target sequence. In other embodiments, the nickase activity is directed to one or more sequences on the ceDNA vectors themselves, for example, to loosen the sequence constraint such that the HDR template is exposed for HDR interaction with the genomic sequence of the target gene.

In certain embodiments, it is contemplated that the nickase cuts at least 1 site, at least 2 sites, at least 3 sites, at least 4 sites, at least 5 sites, at least 6 sites, at least 7 sites, at least 8 sites, at least 9 sites, at least 10 sites or more on the desired nucleic acid sequence (e.g., one or more regions of the ceDNA vector). In another embodiment, it is contemplated that the nickase cuts at 1 and/or 2 sites via trans-nicking. Trans-nicking can enhance genomic editing by HDR, which is high-fidelity, introduces fewer errors, and thus reduces unwanted off-target effects.

In some embodiments, a ceDNA vector for insertion of a transgene into a GSH locus, comprising a HA-L transgene HA-R, as disclosed herein can also encode an RNA-guided endonuclease that is mutated with respect to a corresponding wild-type enzyme such that the mutated endonuclease lacks the ability to cleave one strand of a target polynucleotide containing a target sequence.

In some embodiments, a gene editing cassette can comprise a nucleic acid sequence encoding the RNA-guided endonuclease, which is codon optimized for expression in particular cells, such as eukaryotic cells. The eukaryotic cells can be derived from a particular organism, such as a mammal. Non-limiting examples of mammals can include human, mouse, rat, rabbit, dog, or non-human primate. In general, codon optimization refers to a process of modifying a nucleic acid sequence for enhanced expression in the host cells of interest by replacing at least one codon (e.g., about or more than about 1, 2, 3, 4, 5, 10, 15, 20, 25, 50, or more codons) of the native sequence with codons that are more frequently or most frequently used in the genes of that host cell while maintaining the native amino acid sequence.

In some embodiments, a gene editing cassette can comprise a RNA-guided endonuclease which is part of a fusion protein comprising one or more heterologous protein domains (e.g., about or more than about 1, 2, 3, 4, 5, 6, 7, 8, 9, 10, or more domains in addition to the endonuclease). An RNA-guided endonuclease fusion protein can comprise any additional protein sequence, and optionally a linker sequence between any two domains. Examples of protein domains that can be fused to an RNA-guided endonuclease include, without limitation, epitope tags, reporter gene sequences, purification tags, fluorescent proteins and protein domains having one or more of the following activities: methylase activity, demethylase activity, transcription activation activity, transcription repression activity, transcription release factor activity, histone modification activity, RNA cleavage activity and nucleic acid binding activity. Non-limiting examples of epitope tags include histidine (His) tags, V5 tags, FLAG tags, influenza hemagglutinin (HA) tags, Myc tags, VSV-G tags, glutathione-S-transferase (GST), chitin binding protein (CBP), maltose binding protein (MBP), poly(NANP), tandem affinity purification (TAP) tag, myc, AcV5, AU1, AU5, E, ECS, E2, nus, Softag 1, Softag 3, Strep, SBP, Glu-Glu, HSV, KT3, S, SI, T7, biotin carboxyl carrier protein (BCCP), calmodulin, and thioredoxin (Trx) tags. Examples of reporter genes include, but are not limited to, glutathione-S-transferase (GST), horseradish peroxidase (HRP), chloramphenicol acetyltransferase (CAT) beta-galactosidase, beta-glucuronidase, luciferase, green fluorescent proteins (e.g., GFP, GFP-2, tagGFP, turboGFP, sfGFP, EGFP, Emerald, Azami Green, Monomeric Azami Green, CopGFP, AceGFP, ZsGreen1), HcRed, DsRed, cyan fluorescent protein (CFP), yellow fluorescent proteins (e.g., YFP, EYFP, Citrine, Venus YPet, PhiYFP, ZsYellow1), cyan fluorescent proteins (e.g., ECFP, Cerulean, CyPet AmCyan1, Midoriishi-Cyan) red fluorescent proteins (e.g., mKate, mKate2, mPlum, DsRed monomer, mCherry, mRFP1, DsRed-Express, DsRed2, HcRed-Tandem, HcRed1, AsRed2, eqFP611, mRaspberry, mStrawberry, Jred), orange fluorescent proteins (e.g., mOrange, mKO, Kusabira-Orange, monomeric Kusabira-Orange, mTangerine, tdTomato) and autofluorescent proteins including blue fluorescent protein (BFP). An RNA-guided endonuclease can be fused to a gene sequence encoding a protein or a fragment of a protein that binds DNA molecules or binds to other cellular molecules, including but not limited to maltose binding protein (MBP), S-tag, Lex A DNA binding domain (DBD) fusions, GAL4 DNA binding domain fusions, and herpes simplex virus (HSV) BP16 protein fusions. In some embodiments, a tagged endonuclease is used to identify the location of a target sequence.

It is contemplated herein that at least two (e.g., at least 3, at least 4, at least 5, at least 6, at least 7, at least 8, at least 9, at least 10, at least 12, at least 15 or more) different Cas enzymes are administered or are in contact with a cell at substantially the same time. Any combination of double-stranded break-inducing Cas enzymes, Cas nickases, catalytically inactive Cas enzymes (e.g., dCas9), modified Cas enzymes, truncated Cas9, etc. are contemplated for use in combination with the methods and compositions described herein, in particular, with ceDNA vectors comprising a transgene flanked by a HA-L and a HA-R, where the ceDNA vector does not comprise a gene editing cassette as disclosed herein.

In some embodiments, a gene editing cassette in ceDNA vector comprising a transgene flanked by a HA-L and a HA-R, where the gene edting cassette comprises a nucleic acid-guided endonuclease, such as a DNA-guided endonuclease. See, e.g., Varshney and Burgess Genome Biol. 17:187 (2016). In one embodiment, an enzyme involved in DNA repair and/or replication may be fused to an endonuclease to form a DNA-guided nuclease. One nonlimiting example is the fusion of flap endonuclease 1 (FEN-1) to the FokI endonuclease (Xu et al., Genome Biol. 17:186 (2016). In another embodiment, naturally-occurring DNA-guided nucleases may be used. Nonlimiting examples of such naturally-occurring nucleases are prokaryotic endonucleases from the Argonaute protein family (Kropocheva et al., FEBS Open Bio. 8(S1): P01-074 (2018). In some embodiments, the nucleic acid-guided endonuclease is a “single-base editor”, which is a chimeric protein composed of a DNA targeting module and a catalytic domain capable of modifying a single type of nucleotide base (Rusk, N, Nature Methods 15:763 (2018); Eid et al., Biochem J. 475(11): 1955-64 (2018)). Because such single-base editors do not generate double-strand breaks in the target DNA to effect the editing of the DNA base, the generation of insertions and deletions (e.g., indels) is limited, thus improving the fidelity of the editing process. Different types of single base editors are known. For example, cytidine deaminases (enzymes that catalyze the conversion of cytosine into uracil) may be coupled to nucleases such as APOBEC-dCas9—where APOBEC contributes the cytidine deaminase functionality and is guided by dCas9 to deaminate a specific cytidine to uracil. The resulting U-G mismatches are resolved via repair mechanisms and form U-A base pairs, which translate into C-to-T point mutations (Komor et al., Nature 533: 420-424 (2016); Shimatani et al., Nat. Biotechnol. 35: 441-443 (2017)). Adenine deaminase-based DNA single base editors have been engineered. They deaminate adenosine to form inosine, which can base pair with cytidine and be corrected to guanine such that an A-T pair may be converted to a G-C pair. Examples of such editors include TadA, ABE5.3, ABE7.8, ABE7.9, and ABE7.10 (Gaudelli et al., Nature 551: 464-471 (2017).

### (iv) CRISPR/Cas Systems

In some embodiments, a gene editing cassette in ceDNA vector comprising a transgene flanked by a HA-L and a HA-R, where the gene editing cassette comprises a CRISPR-system. As known in the art, a CRISPR-CAS9 system is a particular set of nucleic-acid guided-nuclease-based systems that includes a combination of protein and ribonucleic acid (“RNA”) that can alter the genetic sequence of an organism. The CRISPR-CAS9 system continues to develop as a powerful tool to modify specific deoxyribonucleic acid (“DNA”) in the genomes of many organisms such as microbes, fungi, plants, and animals. For example, mouse models of human disease can be developed quickly to study individual genes much faster, and easily change multiple genes in cells at once to study their interactions. One of ordinary skill in the art may select between a number of known CRISPR systems such as Type I, Type II, and Type III. Type II CRISPR-CAS system has a well-known mechanism including three components: (1) a crDNA molecule, which is called a “guide sequence” or “targeter-RNA”; (2) a “tracr RNA” or “activator-RNA”; and (3) a protein called Cas9.

To alter the DNA molecule, a number of interactions occur in the system including: (1) the guide sequence binding by specific base pairing to a specific sequence of DNA of interest (“target DNA”), (2) the guide sequence binds by specific base pairing at another sequence to an activator-RNA, and (3) activator-RNA interacts with the Cas protein (e.g., Cas9 protein), which then acts as a nuclease to cut the target DNA at a specific site. Suitable systems for use in accordance with ceDNA vectors in accordance with the present disclosure are further described in Van Nierop, et al. Stimulation of homology-directed gene targeting at an endogenous human locus by a nicking endonuclease, Nucleic Acid Research, August 2009 and Ran et al., Double nicking by RNA-guided CRISPR Cas9 for enhanced genome editing specificity.

ceDNA vectors in accordance with the present disclosure can be designed to include nucleotides encoding one or more components of these systems such as the guide sequence, tracr RNA, or Cas (e.g., Cas9). In certain embodiments, a single promoter drives expression of a guide sequence and tracr RNA, and a separate promoter drives Cas (e.g., Cas9) expression. One of skill in the art will appreciate that certain Cas nucleases require the presence of a protospacer adjacent motif (PAM) adjacent to a target nucleic acid sequence. In some embodiments, the PAM may be adjacent to or within 1, 2, 3, or 4 nucleotides of the 3′ end of the target sequence. The length and the sequence of the PAM can depend on the particular Cas protein. Exemplary PAM sequences include NGG, NGGNG, NG, NAAAAN, NNAAAAAW, NNNNACA, GNNNCNNA, TTN and NNNNGATT (wherein N is defined as any nucleotide and W is defined as either A or T). In some embodiments, the PAM sequence can be on the guide RNA, for example, when editing RNA.

In some embodiments, a gene editing cassette in ceDNA vector comprising a transgene flanked by a HA-L and a HA-R, where the gene edting cassette comprises a RNA-guided nuclease, including Cas and Cas9 are suitable for use in ceDNA vectors designed to provide one or more components for genome engineering using the CRISPR-Cas9 system See e.g. US publication 2014/0170753 herein incorporated by reference in its entirety. CRISPR-Cas 9 provides a set of tools for Cas9-mediated genome editing via non-homologous end joining (NHEJ) or homology-directed repair (HDR) in mammalian cells, as well as generation of modified cell lines for downstream functional studies. To minimize off-target cleavage, the CRISPR-Cas9 system may include a double-nicking strategy using the Cas9 nickase mutant with paired guide RNAs. This system is known in the art, and described in, for example, Ran et al., Genome engineering using the CRISPR-Cas9 system, Nature Protocols, 24 Oct. 2013, and Zhang, et al., Efficient precise knockin with a double cut HDR donor after CRISPR/Cas9-mediated double-stranded DNA cleavage, Genome Biology, 2017 (both references are herein incorporated by reference in their entirety).

In certain embodiments, a gene editing cassette in ceDNA vector comprising a transgene flanked by a HA-L and a HA-R, where the gene edting cassette comprises a nuclease and guide RNAs that are directed to a ceDNA sequence or the HA-L or HA-R regions. For example, a nicking CAS, such as nCAS9 D10A can be used to increase the efficiency of gene editing. The guide RNAs can direct nCAS nicking of the ceDNA thereby releasing torsional constraints of ceDNA for more efficient gene repair and/or expression. Using a nicking nuclease relieves the torsional constraints while retaining sequence and structural integrity allowing the nicked DNA can persist in the nucleus. The guide RNAs can be directed to the same strand of DNA or the complementary strand. The guide RNAs can be directed to e.g., the ITRS, or sequences proceeding promoters, or homology domains etc.

In one embodiment, the RNA-guided endonuclease is a CRISPR enzyme, such as a Cas protein. Non-limiting examples of Cas proteins include Cas1, Cas1B, Cas2, Cas3, Cas4, Cas5, Cas5e (CasD), Cas6, Cas6e, Cas6f, Cas7, Cas8, Cas8a1, Cas8a2, Cas8b, Cas8c, Cas9 (also known as Csn1 and Csx12), Cas10, Cas10d, Cas13, Cas13a, Cas13c, CasF, CasH, Csy1, Csy2, Csy3, Cse1, Cse2, Cse3, Cse4, Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx11, Csx16, CsaX, Csz1, Csx3, Csx1, Csx15, Csf1, Csf2, Csf3, Csf4, Cul966, Cpf1, C2c1, C2c3, homologs thereof, or modified versions thereof. In one embodiment, the Cas protein is Cas9. In another embodiment, the Cas protein is nuclease-dead Cas9 (dCas9) or a Cas9 nickase. In one embodiment, the Cas protein is a nicking Cas enzyme (nCas).

In one embodiment, the Cas9 nickase comprises nCas9 D10A. For example, an aspartate-to-alanine substitution (D10A) in the RuvC I catalytic domain of Cas9 from S. pyogenes converts Cas9 from a nuclease that cleaves both strands to a nickase (cleaves a single strand). Other examples of mutations that render Cas9 a nickase include, without limitation, H840A, N854A, and N863A. In some embodiments, a Cas9 nickase can be used in combination with guide sequence(s), e.g., two guide sequences, which target respectively sense and antisense strands of the DNA target. This combination allows both strands to be nicked and used to induce non-homologous end joining (NHEJ) repair.

In some embodiments, a gene editing cassette in ceDNA vector comprising a transgene flanked by a HA-L and a HA-R, where the gene edting cassette comprises a RNA-guided endonuclease which is Cas13. A catalytically inactive Cas13 (dCas13) can be used to edit mRNA sequences as described in e.g., Cox, D et al. RNA editing with CRISPR-Cas13 Science (2017) DOI: 10.1126/science.aaq0180, which is herein incorporated by reference in its entirety.

In some embodiments, a gene editing cassette in ceDNA vector comprising a transgene flanked by a HA-L and a HA-R comprises nucleic acid encoding an endonuclease, such as Cas9 (e.g., disclosed asSEQ ID NO: 829 in PCT/US18/64242, which is incorporated herein in its entirety by reference), or an amino acid or functional fragment of a nuclease having at least 60%, more preferably at least 65%, more preferably at least 70%, more preferably at least 75%, more preferably at least 80%, more preferably at least 85%, even more preferably at least 90%, and most preferably at least 95% sequence identity to SEQ ID NO:829 (Cas9) or consisting of SEQ ID NO: 829, as disclosed as in PCT/US18/64242, which is incorporated herein in its entirety by reference. In certain embodiments, Cas 9 includes one or more mutations in a catalytic domain rendering the Cas 9 a nickase that cleaves a single DNA strand, such as those described in U.S. Patent Publication No. 2017-0191078-A9 (incorporated by reference in its entirety).

In some embodiments, the ceDNA vectors of the present disclosure are suitable for use in systems and methods based on RNA-programmed Cas9 having gene-targeting and genome editing functionality. For example, the ceDNA vectors of the present disclosure are suitable for use with Clustered Regularly Interspaced Short Palindromic Repeats or the CRISPR associated (Cas) systems for gene targeting and gene editing. CRISPR cas9 systems are known in the art and described, e.g., in U.S. patent application Ser. No. 13/842,859 filed on March 2013, and U.S. Pat. Nos. 8,697,359, 8,771,945, 8,795,965, 8,865,406, 8,871,445 all of which are herein incorporated by reference in their entirety.

It is also contemplated herein that Cas9, a Cas9 nickase, or a deactivated Cas9 (dCas9, or also referred to a nuclease dead Cas9 or “catalytically inactive”) are also prepared as fusion proteins with FokI, such that gene editing or gene expression modulation occurs upon formation of FokI heterodimers.

Further, dCas9 can be used to activate (CRISPRa) or inhibit (CRISPRi) expression of a desired gene at the level of regulatory sequences upstream of the target gene sequence. CRISPRa and CRISPRi can be performed, for example, by fusing dCas9 with an effector region (e.g., dCas9/effector fusion) and supplying a guide RNA that directs the dCas9/effector fusion protein to bind to a sequence upstream of the desired or target gene (e.g., in the promoter region). Since dCas9 has no nuclease activity, it remains bound to the target site in the promoter region and the effector portion of the dCas9/effector fusion protein can recruit transcriptional activators or repressors to the promoter site. As such, one can activate or reduce gene expression of a target gene as desired. Previous work in the literature indicates that the use of a plurality of guide RNAs co-expressed with dCas9 can increase expression of a desired gene (see e.g., Maeder et al. CRISPR RNA-guided activation of endogenous human genes Nat Methods 10(10):977-979 (2013). In some embodiments, it is desirable to permit inducible repression of a desired gene. This can be achieved, for example, by using guide RNA binding sites in promoter regions upstream of the transcription start site (see e.g., Gao et al. Complex transcriptional modulation with orthogonal and inducible dCas9 regulators. Nature Methods (2016)). In some embodiments, a nuclease dead version of a DNA endonuclease (e.g., dCas9) can be used to inducibly activate or increase expression of a desired gene, for example, by introduction of an agent that interacts with an effector domain (e.g., a small molecule or at least one guide RNA) of a dCas9/effector fusion protein. In other embodiments, it is also contemplated herein that dCas9 can be fused to a chemical- or light-inducible domain, such that gene expression can be modulated using extrinsic signals. In one embodiment, inhibition of a target gene's expression is performed using dCas9 fused to a KRAB repressor domain, which may be beneficial for improved inhibition of gene expression in mammalian systems and have few off-target effects. Alternatively, transcription-based activation of a gene can be performed using a dCas9 fused to the omega subunit of RNA polymerase, or the transcriptional activators VP64 or p65.

Accordingly, in some embodiments, the methods and compositions described herein, e.g., ceDNA vectors can comprise and/or be used to deliver CRISPRi (CRISPR interference) and/or CRISPRa (CRISPR activation) systems to a host cell. CRISPRi and CRISPRa systems comprise a deactivated RNA-guided endonuclease (e.g., Cas9) that cannot generate a double strand break (DSB). This permits the endonuclease, in combination with the guide RNAs, to bind specifically to a target sequence in the genome and provide RNA-directed reversible transcriptional control. In one embodiment, the ceDNA vector comprises a nucleic acid encoding a nuclease and/or a guide RNA but does not comprise a homology directed repair template or corresponding homology arms.

In some embodiments of CRISPRi, the endonuclease can comprise a KRAB effector domain. Either with or without the KRAB effector domain, the binding of the deactivated nuclease to the genomic sequence can, e.g., block transcription initiation or progression and/or interfere with the binding of transcriptional machinery or transcription factors.

In CRISPRa, the deactivated endonuclease can be fused with one or more transcriptional activation domains, thereby increasing transcription at or near the site targeted by the endonuclease. In some embodiments, CRISPRa can further comprise gRNAs which recruit further transcriptional activation domains. sgRNA design for CRISPRi and CRISPRa is known in the art (see, e.g., Horlbeck et al. eLife. 5, e19760 (2016); Gilbert et al., Cell. 159,647-661 (2014); and Zalatan et al., Cell. 160,339-350 (2015); each of which is incorporated by reference here in its entirety). CRISPRi and CRISPRa-compatible sgRNA can also be obtained commercially for a given target (see, e.g., Dharmacon; Lafayette, Colo.). Further description of CRISPRi and CRISPRa can be found, e.g., in Qi et al., Cell. 152,1173-1183 (2013); Gilbert et al., Cell. 154, 442-451 (2013); Cheng et al., Cell Res. 23,1163-1171 (2013); Tanenbaum et al. Cell. 159,635-646 (2014); Konermann et al., Nature. 517,583-588 (2015); Chavez et al., Nat. Methods. 12,326-328 (2015); Liu et al., Science. 355 (2017); and Goyal et al., Nucleic Acids Res. (2016); each of which is incorporated by reference herein in its entirety.

Accordingly, in some embodiments described herein is a gene editing cassette in ceDNA vector comprising a transgene flanked by a HA-L and a HA-R, where the gene edting cassette comprises a deactivated endonuclease, e.g., RNA-guided endonuclease and/or Cas9, wherein the deactivated endonuclease lacks endonuclease activity, but retains the ability to bind DNA in a site-specific manner, e.g., in combination with one or more guide RNAs and/or sgRNAs. In some embodiments, the vector can further comprise one or more tracrRNAs, guide RNAs, or sgRNAs. In some embodiments, the deactivated endonuclease can further comprise a transcriptional activation domain. In some embodiments, ceDNA vectors of the present disclosure are also useful for deactivated nuclease systems, such as CRISPRi or CRISPRa dCas systems, nCas, or Cas13 systems, all well known in the art.

It is also contemplated herein that the vectors described herein can be used in combination with dCas9 to visualize genomic loci in living cells (see e.g., Ma et al. Multicolor CRISPR labeling of chromosomal loci in human cells PNAS 112(10):3002-3007 (2015)). CRISPR mediated visualization of the genome and its organization within the nucleus is also called the 4-D nucleome. In one embodiment, dCas9 is modified to comprise a fluorescent tag. Multiple loci can be labeled in distinct colors, for example, using orthologs that are each fused to a different fluorescent label. This technique can be expanded to study genome structure, for example, by using guide RNAs that bind Alu sequences to aid in mapping the location of guide RNA-specified repeats (see e.g., McCaffrey et al. Nucleic Acids Res 44(2):e11 (2016)). Thus, in some embodiments, mapping of clinically significant loci is contemplated herein, for example, for the identification and/or diagnosis of Huntington's disease, among others. Methods of performing genome visualization or genetic screens with a ceDNA vector(s) encoding a gene editing system are known in the art and/or are described in, for example, Chen et al. Cell 155:1479-1491 (2013); Singh et al. Nat Commun 7:1-8 (2016); Korkmaz et al. Nat Biotechnol 34:1-10 (2016); Hart et al. Cell 163:1515-1526 (2015); the contents of each of which are incorporated herein by reference in their entirety.

In some embodiments, it may be desirable to edit a single base in the genome, for example, modifying a single nucleotide polymorphism associated with a particular disease (see e.g., Komor, A C et al. Nature 533:420-424 (2016); Nishida, K et al. Targeted nucleotide editing using hybrid prokaryotic and vertebrate adaptive immune systems. Science (2016)). Single nucleotide base editing makes use of base-converting enzyme tethered to a catalytically inactive endonuclease (e.g., nuclease dead Cas9) that does not cut the target gene locus. After the base conversion by a base editing enzyme, the system makes a nick on the opposite, unedited strand, which is repaired by the cell's own DNA repair mechanisms. This results in the replacement of the original nucleotide, which is now a “mismatched nucleotide,” thus completing the conversion of a single nucleotide base pair. Endogenous enzymes are available for effecting the conversion of G/C nucleotide pairs to A/T nucleotide pairs, for example, cytidine deaminase, however there is no endogenous enzyme for catalyzing the reverse conversion of A/T nucleotide pairs to G/C ones. Adenine deaminases (e.g., TadA), that usually only act on RNA to convert adenine to inosine, have been evolutionarily selected for in bacterial systems to identify adenine deaminase mutants that act on DNA to convert adenosine to inosine (see e.g., Gaudelli et al Nature (2017), in press doi:10.1038/nature24644, the contents of which are incorporated by reference in its entirety).

In some embodiments, dCas9 or a modified Cas9 with a nickase function can be fused to an enzyme having a base editing function (e.g., cytidine deaminase APOBEC1 or a mutant TadA). The base editing efficiency can be further improved by including an inhibitor of endogenous base excision repair systems that remove uracil from the genomic DNA. See Gaudelli et al. (2017) programmable base editing of A-T to G-C in genomic DNA without DNA cleavage, Nature Published online 25 Oct. 2017, herein incorporated by reference in its entirety.

It is also contemplated herein that the desired endonuclease is modified by addition of ubiquitin or a polyubiquitin chain. In some embodiments, the ubiquitin can be a ubiquitin-like protein (UBL). Non-limiting examples of ubiquitin-like proteins include small ubiquitin-like modifier (SUMO), ubiquitin cross-reactive protein (UCRP, also known as interferon-stimulated gene 15 (ISG-15)), ubiquitin-related modifier-1 (URM1), neuronal-precursor-cell-expressed developmentally downregulated protein-8 (NEDD8, also called Rubl in S. cerevisiae), human leukocyte antigen F-associated (FAT 10), autophagy-8 (ATG8) and -12 (ATG12), Fau ubiquitin-like protein (FUB1), membrane-anchored UBL (MUB), ubiquitin fold-modifier-1 (UFM1), and ubiquitin-like protein-5 (UBL5).

A gene editing cassette in ceDNA vector comprising a transgene flanked by a HA-L and a HA-R, where the gene edting cassette comprises tcan encode for modified DNA endonucleases as described in e.g., Fu et al. Nat Biotechnol 32:279-284 (2013); Ran et al. Cell 154:1380-1389 (2013); Mali et al. Nat Biotechnol 31:833-838 (2013); Guilinger et al. Nat Biotechnol 32:577-582 (2014); Slaymaker et al. Science 351:84-88 (2015); Klenstiver et al. Nature 523:481-485 (2015); Bolukbasi et al. Nat Methods 12:1-9 (2015); Gilbert et al. Cell 154; 442-451 (2012); Anders et al. Mol Cell 61:895-902 (2016); Wright et al. Proc Natl Acad Sci USA 112:2984-2989 (2015); Truong et al. Nucleic Acids Res 43:6450-6458 (2015); the contents of each of which are incorporated herein by reference in their entirety.

### (v) MegaTALS

In some embodiments, a gene editing cassette in ceDNA vector comprising a transgene flanked by a HA-L and a HA-R, where the gene edting cassette comprises an endonuclease which is a megaTAL. MegaTALs are engineered fusion proteins which comprise a transcription activator-like (TAL) effector domain and a meganuclease domain. MegaTALs retain the ease of target specificity engineering of TALs while reducing off-target effects and overall enzyme size and increasing activity. MegaTAL construction and use is described in more detail in, e.g., Boissel et al. 2014 Nucleic Acids Research 42(4):2591-601 and Boissel 2015 Methods Mol Biol 1239:171-196; each of which is incorporated by reference herein in its entirety. Protocols for megaTAL-mediated gene knockout and gene editing are known in the art, see, e.g., Sather et al. Science Translational Medicine 2015 7(307):ra156 and Boissel et al. 2014 Nucleic Acids Research 42(4):2591-601; each of which is incorporated by reference herein in its entirety. MegaTALs can be used as an alternative endonuclease in any of the methods and compositions described herein.

### (vi) Multiplex Modulation of Gene Expression and Complex Systems

The lack of size limitations of the ceDNA vectors as described herein are especially useful in multiplexed editing, CRISPRa or CRISPRi because multiple guide RNAs can be expressed from the same ceDNA vector, if desired. CRISPR is a robust system and the addition of multiple guide RNAs does not substantially alter the efficiency of gene editing, CRISPRa, CRISPRi or CRISPR mediated labeling of nucleic acids. As described elsewhere, the plurality of guide RNAs can be under the control of a single promoter (e.g., a polycistronic transcript) or under the control of a plurality of promoters (e.g., at least 2, at least 3, at least 4, at least 5, at least 6, etc. up to a limit of a 1:1 ratio of guide RNA:promoter sequences).

The multiplex CRISPR/Cas9-Based System takes advantage of the simplicity and low cost of sgRNA design and may be helpful in exploiting advances in high-throughput genomic research using CRISPR/Cas9 technology. For example, the ceDNA vectors described herein are useful in expressing Cas9 and numerous single guide RNAs (sgRNAs) in difficult cell lines, as well as insertion of the transgene located between the HA-L and HA-R regions into the genome of a host cell. The multiplex CRISPR/Cas9-Based System may be used in the same ways as the CRISPR/Cas9-Based System described above. Multiplex CRISPR/Cas can be performed as described in Cong, L et al. Science 819 (2013); Wang et al. Cell 153:910-918 (2013); Ma et al. Nat Biotechnol 34:528-530 (2016); the contents of each of which are incorporated herein by reference in their entirety.

(vii) Guide RNAs (gRNAs)

In general, a guide sequence is any polynucleotide sequence having sufficient complementarity with a target polynucleotide sequence to hybridize with the target sequence and direct sequence-specific targeting of an RNA-guided endonuclease complex to the selected genomic target sequence. In some embodiments, a guide RNA binds and e.g., a Cas protein can form a ribonucleoprotein (RNP), for example, a CRISPR/Cas complex.

In some embodiments, the gene editing cassette of a ceDNA vector for insertion of a transgene into a GSH locus disclosed herein comprises a guide RNA (gRNA) sequence that comprises a targeting sequence that directs the gRNA sequence to a desired site in the genome, fused to a crRNA and/or tracrRNA sequence that permit association of the guide sequence with the RNA-guided endonuclease. In some embodiments, the degree of complementarity between a guide sequence and its corresponding target sequence, when optimally aligned using a suitable alignment algorithm, is at least 50%, 60%, 75%, 80%, 85%, 90%, 95%, 97.5%, 99%, or more. Optimal alignment can be determined with the use of any suitable algorithm for aligning sequences, such as the Smith-Waterman algorithm, the Needleman-Wunsch algorithm, algorithms based on the Burrows-Wheeler Transform (e.g., the Burrows Wheeler Aligner), ClustalW, Clustal X, BLAT, Novoalign (Novocraft Technologies, ELAND (Illumina, San Diego, Calif.), SOAP, and Maq. In some embodiments, a guide sequence is 5, 10, 11, 12, 13, 14, 15, 16, 17, 18, 19, 20, 21, 22, 23, 24, 25, 26, 27, 28, 29, 30, 35, 40, 45, 50, 75, or more nucleotides in length. It is contemplated herein that the targeting sequence of the guide RNA and the target sequence on the target nucleic acid molecule can comprise 1, 2, 3, 4, 5, 6, 7, 8, 9, or 10 mismatches. In some embodiments, the guide RNA sequence comprises a palindromic sequence, for example, the self-targeting sequence comprises a palindrome. The targeting sequence of the guide RNA is typically 19-21 base pairs long and directly precedes the hairpin that binds the entire guide RNA (targeting sequence+ hairpin) to a Cas such as Cas9. Where a palindromic sequence is employed as the self-targeting sequence of the guide RNA, the inverted repeat element can be e.g., 9, 10, 11, 12, or more nucleotides in length. Where the targeting sequence of the guide RNA is most often 19-21 bp, a palindromic inverted repeat element of 9 or 10 nucleotides provides a targeting sequence of desirable length. The Cas9-guide RNA hairpin complex can then recognize and cut any nucleotide sequence (DNA or RNA) e.g., a DNA sequence that matches the 19-21 base pair sequence and is followed by a “PAM” sequence e.g., NGG or NGA, or other PAM.

The ability of a guide sequence to direct sequence-specific binding of an RNA-guided endonuclease complex to a target sequence can be assessed by any suitable assay. For example, the components of an RNA-guided endonuclease system sufficient to form an RNA-guided endonuclease complex can be provided to a host cell having the corresponding target sequence, such as by transfection with vectors encoding the components of the RNA-guided endonuclease sequence, followed by an assessment of preferential cleavage within the target sequence, such as by Surveyor assay (Transgenomic™, New Haven, Conn.). Similarly, cleavage of a target polynucleotide sequence can be evaluated in a test tube by providing the target sequence, components of an RNA-guided endonuclease complex, including the guide sequence to be tested and a control guide sequence different from the test guide sequence, and comparing binding or rate of cleavage at the target sequence between the test and control guide sequence reactions. One of ordinary skill in the art will appreciate that other assays can also be used to test gRNA sequences.

A guide sequence can be selected to target any target sequence. In some embodiments, the target sequence is a sequence within a genome of a cell. In some embodiments, the target sequence is the sequence encoding a first guide RNA in a self-cloning plasmid, as described herein. Typically, the target sequence in the genome will include a protospacer adjacent (PAM) sequence for binding of the RNA-guided endonuclease. It will be appreciated by one of skill in the art that the PAM sequence and the RNA-guided endonuclease should be selected from the same (bacterial) species to permit proper association of the endonuclease with the targeting sequence. For example, the PAM sequence for CAS9 is different than the PAM sequence for cpF1. Design is based on the appropriate PAM sequence. To prevent degradation of the guide RNA, the sequence of the guide RNA should not contain the PAM sequence. In some embodiments, the length of the targeting sequence in the guide RNA is 12 nucleotides; in other embodiments, the length of the targeting sequence in the guide RNA is 13, 14, 15, 16, 17, 18, 19, 20, 21, 22, 23, 24, 25, 26, 27, 28, 29, 30, 35 or 40 nucleotides. The guide RNA can be complementary to either strand of the targeted DNA sequence. In some embodiments, when modifying the genome to include an insertion or deletion, the gRNA can be targeted closer to the N-terminus of a protein coding region.

It will be appreciated by one of skill in the art that for the purposes of targeted cleavage by an RNA-guided endonuclease, target sequences that are unique in the genome are preferred over target sequences that occur more than once in the genome. Bioinformatics software can be used to predict and minimize off-target effects of a guide RNA (see e.g., Naito et al. “CRISPRdirect: software for designing CRISPR/Cas guide RNA with reduced off-target sites” Bioinformatics (2014), epub; Heigwer, F., et al. “E-CRISP: fast CRISPR target site identification” Nat. Methods 11, 122-123 (2014); Bae et al. “Cas-OFFinder: a fast and versatile algorithm that searches for potential off-target sites of Cas9 RNA-guided endonucleases” Bioinformatics 30(10):1473-1475 (2014); Aach et al. “CasFinder: Flexible algorithm for identifying specific Cas9 targets in genomes” BioRxiv (2014), among others).

Target sequences for different Cas9 are disclosed as SEQ ID NO: 590-601 in International Patent Application PCT/US18/49996 filed Dec. 6, 2018, which is incorporated herein in its entirety.

In general, a “crRNA/tracrRNA fusion sequence,” as that term is used herein refers to a nucleic acid sequence that is fused to a unique targeting sequence and that functions to permit formation of a complex comprising the guide RNA and the RNA-guided endonuclease. Such sequences can be modeled after CRISPR RNA (crRNA) sequences in prokaryotes, which comprise (i) a variable sequence termed a “protospacer” that corresponds to the target sequence as described herein, and (ii) a CRISPR repeat. Similarly, the tracrRNA (“transactivating CRISPR RNA”) portion of the fusion can be designed to comprise a secondary structure similar to the tracrRNA sequences in prokaryotes (e.g., a hairpin), to permit formation of the endonuclease complex. In some embodiments, the fusion has sufficient complementarity with a tracrRNA sequence to promote one or more of: (1) excision of a guide sequence flanked by tracrRNA sequences in a cell containing the corresponding tracr sequence; and (2) formation of an endonuclease complex at a target sequence, wherein the complex comprises the crRNA sequence hybridized to the tracrRNA sequence. In general, degree of complementarity is with reference to the optimal alignment of the crRNA sequence and tracrRNA sequence, along the length of the shorter of the two sequences. Optimal alignment can be determined by any suitable alignment algorithm, and can further account for secondary structures, such as self-complementarity within either the tracrRNA sequence or crRNA sequence. In some embodiments, the degree of complementarity between the tracrRNA sequence and crRNA sequence along the length of the shorter of the two when optimally aligned is about or more than about 25%, 30%, 40%, 50%, 60%, 70%, 80%, 90%, 95%, 97.5%, 99%, or higher. In some embodiments, the tracrRNA sequence is at least 5, 6, 7, 8, 9, 10, 11, 12, 13, 14, 15, 16, 17, 18, 19, 20, 25, 30, 40, 50, 60, 70, 80, 90, 100, or more nucleotides in length (e.g., 70-80, 70-75, 75-80 nucleotides in length). In one embodiment, the crRNA is less than 60, less than 50, less than 40, less than 30, or less than 20 nucleotides in length. In other embodiments, the crRNA is 30-50 nucleotides in length; in other embodiments the crRNA is 30-50, 35-50, 40-50, 40-45, 45-50 or 50-55 nucleotides in length. In some embodiments, the crRNA sequence and tracrRNA sequence are contained within a single transcript, such that hybridization between the two produces a transcript having a secondary structure, such as a hairpin. In some embodiments, the loop forming sequences for use in hairpin structures are four nucleotides in length, for example, the sequence GAAA. However, longer or shorter loop sequences can be used, as can alternative sequences. The sequences preferably include a nucleotide triplet (for example, AAA), and an additional nucleotide (for example C or G). Examples of loop forming sequences include CAAA and AAAG. In one embodiment, the transcript or transcribed gRNA sequence comprises at least one hairpin. In one embodiment, the transcript or transcribed polynucleotide sequence has at least two or more hairpins. In other embodiments, the transcript has two, three, four or five hairpins. In a further embodiment, the transcript has at most five hairpins. In some embodiments, the single transcript further includes a transcription termination sequence, such as a polyT sequence, for example six T nucleotides. Non-limiting examples of single polynucleotides comprising a guide sequence, a crRNA sequence, and a tracr sequence are disclosed as SEQ ID NO: 602-607 in International Patent Application PCT/US18/49996, filed Dec. 6, 2018, which is incorporated herein in its entirety.

In some embodiments, a guide RNA can comprise two RNA molecules and is referred to herein as a “dual guide RNA” or “dgRNA.” In some embodiments, the dgRNA may comprise a first RNA molecule comprising a crRNA, and a second RNA molecule comprising a tracrRNA. The first and second RNA molecules may form a RNA duplex via the base pairing between the flagpole on the crRNA and the tracrRNA. When using a dgRNA, the flagpole need not have an upper limit with respect to length.

In other embodiments, a guide RNA can comprise a single RNA molecule and is referred to herein as a “single guide RNA” or “sgRNA.” In some embodiments, the sgRNA can comprise a crRNA covalently linked to a tracrRNA. In some embodiments, the crRNA and tracrRNA can be covalently linked via a linker. In some embodiments, the sgRNA can comprise a stem-loop structure via the base-pairing between the flagpole on the crRNA and the tracrRNA. In some embodiments, a single-guide RNA is at least 50, at least 60, at least 70, at least 80, at least 90, at least 100, at least 110, at least 120 or more nucleotides in length (e.g., 75-120, 75-110, 75-100, 75-90, 75-80, 80-120, 80-110, 80-100, 80-90, 85-120, 85-110, 85-100, 85-90, 90-120, 90-110, 90-100, 100-120, 100-120 nucleotides in length). In some embodiments, a ceDNA vector or composition thereof comprises a nucleic acid that encodes at least 1 gRNA. For example, the second polynucleotide sequence may encode at least 1 gRNA, at least 2 gRNAs, at least 3 gRNAs, at least 4 gRNAs, at least 5 gRNAs, at least 6 gRNAs, at least 7 gRNAs, at least 8 gRNAs, at least 9 gRNAs, at least 10 gRNAs, at least 11 gRNA, at least 12 gRNAs, at least 13 gRNAs, at least 14 gRNAs, at least 15 gRNAs, at least 16 gRNAs, at least 17 gRNAs, at least 18 gRNAs, at least 19 gRNAs, at least 20 gRNAs, at least 25 gRNA, at least 30 gRNAs, at least 35 gRNAs, at least 40 gRNAs, at least 45 gRNAs, or at least 50 gRNAs. The second polynucleotide sequence may encode between 1 gRNA and 50 gRNAs, between 1 gRNA and 45 gRNAs, between 1 gRNA and 40 gRNAs, between 1 gRNA and 35 gRNAs, between 1 gRNA and 30 gRNAs, between 1 gRNA and 25 different gRNAs, between 1 gRNA and 20 gRNAs, between 1 gRNA and 16 gRNAs, between 1 gRNA and 8 different gRNAs, between 4 different gRNAs and 50 different gRNAs, between 4 different gRNAs and 45 different gRNAs, between 4 different gRNAs and 40 different gRNAs, between 4 different gRNAs and 35 different gRNAs, between 4 different gRNAs and 30 different gRNAs, between 4 different gRNAs and 25 different gRNAs, between 4 different gRNAs and 20 different gRNAs, between 4 different gRNAs and 16 different gRNAs, between 4 different gRNAs and 8 different gRNAs, between 8 different gRNAs and 50 different gRNAs, between 8 different gRNAs and 45 different gRNAs, between 8 different gRNAs and 40 different gRNAs, between 8 different gRNAs and 35 different gRNAs, between 8 different gRNAs and 30 different gRNAs, between 8 different gRNAs and 25 different gRNAs, between 8 different gRNAs and 20 different gRNAs, between 8 different gRNAs and 16 different gRNAs, between 16 different gRNAs and 50 different gRNAs, between 16 different gRNAs and 45 different gRNAs, between 16 different gRNAs and 40 different gRNAs, between 16 different gRNAs and 35 different gRNAs, between 16 different gRNAs and 30 different gRNAs, between 16 different gRNAs and 25 different gRNAs, or between 16 different gRNAs and 20 different gRNAs. Each of the polynucleotide sequences encoding the different gRNAs may be operably linked to a promoter. The promoters that are operably linked to the different gRNAs may be the same promoter. The promoters that are operably linked to the different gRNAs may be different promoters. The promoter may be a constitutive promoter, an inducible promoter, a repressible promoter, or a regulatable promoter.

In some experiments, the guide RNAs will target known ZFN sequence targeted regions successful for knock-ins, or knock-out deletions, or for correction of defective genes. Multiple sgRNA sequences that bind known ZFN target regions have been designed and are described in Tables 1-2 of US patent publication 2015/0056705, which is herein incorporated by reference in its entirety, and include for example gRNA sequences for human beta-globin, human, BCLIIA, human KLF1, Human CCR5, Human CXCR4, PPP1R12C, mouse and human HPRT, human albumin, human factor IX human factor VIII, human LRRK2, human Htt, human RH, CFTR, TRAC, TRBC, human PD1, human CTLA-4, HLA c11, HLA A2, HLA A3, HLA B, HLA C, HLA c1, II DBp2. DRA, Tap 1 and 2. Tapasin, DMD, RFX5, etc.)

Modified nucleosides or nucleotides can be present in a guide RNA or mRNA as described herein. An mRNA encoding a guide RNA or a DNA endonuclease (e.g., an RNA-guided nuclease) can comprise one or more modified nucleosides or nucleotides; such mRNAs are called “modified” to describe the presence of one or more non-naturally and/or naturally occurring components or configurations that are used instead of or in addition to the canonical A, G, C, and U residues. In some embodiments, a modified RNA is synthesized with a non-canonical nucleoside or nucleotide, here called “modified.” Modified nucleosides and nucleotides can include one or more of: (i) alteration, e.g., replacement, of one or both of the non-linking phosphate oxygens and/or of one or more of the linking phosphate oxygens in the phosphodiester backbone linkage (an exemplary backbone modification); (ii) alteration, e.g., replacement, of a constituent of the ribose sugar, e.g., of the 2′ hydroxyl on the ribose sugar (an exemplary sugar modification); (iii) wholesale replacement of the phosphate moiety with “dephospho” linkers (an exemplary backbone modification); (iv) modification or replacement of a naturally occurring nucleobase, including with a non-canonical nucleobase (an exemplary base modification); (v) replacement or modification of the ribose-phosphate backbone (an exemplary backbone modification); (vi) modification of the 3′ end or 5′ end of the oligonucleotide, e.g., removal, modification or replacement of a terminal phosphate group or conjugation of a moiety, cap or linker (such 3′ or 5′ cap modifications may comprise a sugar and/or backbone modification); and (vii) modification or replacement of the sugar (an exemplary sugar modification). Unmodified nucleic acids can be prone to degradation by, e.g., cellular nucleases. For example, nucleases can hydrolyze nucleic acid phosphodiester bonds. Accordingly, in one aspect the guide RNAs described herein can contain one or more modified nucleosides or nucleotides, e.g., to introduce stability toward nucleases. In certain embodiments, the mRNAs described herein can contain one or more modified nucleosides or nucleotides, e.g., to introduce stability toward nucleases. In one embodiment, the modification includes 2′-O-methyl nucleotides. In other embodiments, the modification comprises phosphorothioate (PS) linkages.

Examples of modified phosphate groups include, phosphorothioate, phosphoroselenates, borano phosphates, borano phosphate esters, hydrogen phosphonates, phosphoroamidates, alkyl or aryl phosphonates and phosphotriesters. The phosphorous atom in an unmodified phosphate group is achiral. However, replacement of one of the non-bridging oxygens with one of the above atoms or groups of atoms can render the phosphorous atom chiral. The stereogenic phosphorous atom can possess either the “R” configuration (herein Rp) or the “S” configuration (herein Sp). The backbone can also be modified by replacement of a bridging oxygen, (i.e., the oxygen that links the phosphate to the nucleoside), with nitrogen (bridged phosphoroamidates), sulfur (bridged phosphorothioates) and carbon (bridged methylenephosphonates). The replacement can occur at either linking oxygen or at both of the linking oxygens. The phosphate group can be replaced by non-phosphorus containing connectors in certain backbone modifications. In some embodiments, the charged phosphate group can be replaced by a neutral moiety. Examples of moieties which can replace the phosphate group can include, without limitation, e.g., methyl phosphonate, hydroxylamino, siloxane, carbonate, carboxy methyl, carbamate, amide, thioether, ethylene oxide linker, sulfonate, sulfonamide, thioformacetal, formacetal, oxime, methyleneimino, methylenemethylimino, methylenehydrazo, methylenedimethylhydrazo and methyleneoxymethylimino.

Modified nucleosides and nucleotides can include one or more modifications to the sugar group, i.e. at sugar modification. For example, the 2′ hydroxyl group (OH) can be modified, e.g., replaced with a number of different “oxy” or “deoxy” substituents. In some embodiments, modifications to the 2′ hydroxyl group can enhance the stability of the nucleic acid since the hydroxyl can no longer be deprotonated to form a 2′-alkoxide ion. Examples of 2′ hydroxyl group modifications can include alkoxy or aryloxy (OR, wherein “R” can be, e.g., alkyl, cycloalkyl, aryl, aralkyl, heteroaryl or a sugar); poly ethylene glycols (PEG), 0(CH2CH20)nCH2CH2OR wherein R can be, e.g., H or optionally substituted alkyl, and n can be an integer from 0 to 20 (e.g., from 0 to 4, from 0 to 8, from 0 to 10, from 0 to 16, from 1 to 4, from 1 to 8, from 1 to 10, from 1 to 16, from 1 to 20, from 2 to 4, from 2 to 8, from 2 to 10, from 2 to 16, from 2 to 20, from 4 to 8, from 4 to 10, from 4 to 16, and from 4 to 20). In some embodiments, the 2′ hydroxyl group modification can be 2′-0-Me. In some embodiments, the 2′ hydroxyl group modification can be a 2′-fluoro modification, which replaces the 2′ hydroxyl group with a fluoride. In some embodiments, the 2′ hydroxyl group modification can include “locked” nucleic acids (LNA) in which the 2′ hydroxyl can be connected, e.g., by a Ci-6 alkylene or Ci-6 heteroalkylene bridge, to the 4′ carbon of the same ribose sugar, where exemplary bridges can include methylene, propylene, ether, or amino bridges; O-amino (wherein amino can be, e.g., NH2; alkylamino, dialkylamino, heterocyclyl, arylamino, diarylamino, heteroarylamino, or diheteroarylamino, ethylenediamine, or polyamino) and aminoalkoxy, 0(CH2)n-amino, (wherein amino can be, e.g., NH2; alkylamino, dialkylamino, heterocyclyl, arylamino, diarylamino, heteroarylamino, or diheteroarylamino, ethylenediamine, or polyamino). In some embodiments, the 2′ hydroxyl group modification can include “unlocked” nucleic acids (UNA) in which the ribose ring lacks the C2′-C3′ bond. In some embodiments, the 2′ hydroxyl group modification can include the methoxyethyl group (MOE), (OCH2CH2OCH3, e.g., a PEG derivative).

The term “Deoxy” 2′ modifications can include hydrogen (i.e. deoxyribose sugars, e.g., at the overhang portions of partially dsRNA); halo (e.g., bromo, chloro, fluoro, or iodo); amino (wherein amino can be, e.g., —NH2, alkylamino, dialkylamino, heterocyclyl, arylamino, diarylamino, heteroarylamino, diheteroarylamino, or amino acid); NH(CH2CH2NH)nCH2CH2-amino (wherein amino can be, e.g., as described herein), —NHC(O)R (wherein R can be, e.g., alkyl, cycloalkyl, aryl, aralkyl, heteroaryl or sugar), cyano; mercapto; alkyl-thio-alkyl; thioalkoxy; and alkyl, cycloalkyl, aryl, alkenyl and alkynyl, which may be optionally substituted with e.g., an amino as described herein. The sugar modification can comprise a sugar group which can also contain one or more carbons that possess the opposite stereochemical configuration than that of the corresponding carbon in ribose. Thus, a modified nucleic acid can include nucleotides containing e.g., arabinose, as the sugar. The modified nucleic acids can also include abasic sugars. These abasic sugars can also be further modified at one or more of the constituent sugar atoms. The modified nucleic acids can also include one or more sugars that are in the L form, e.g. L-nucleosides.

The modified nucleosides and modified nucleotides described herein, which can be incorporated into a modified nucleic acid, can include a modified base, also called a nucleobase. Examples of nucleobases include, but are not limited to, adenine (A), guanine (G), cytosine (C), and uracil (U). These nucleobases can be modified or wholly replaced to provide modified residues that can be incorporated into modified nucleic acids. The nucleobase of the nucleotide can be independently selected from a purine, a pyrimidine, a purine analog, or pyrimidine analog. In some embodiments, the nucleobase can include, for example, naturally-occurring and synthetic derivatives of a base.

In embodiments employing a dual guide RNA, each of the crRNA and the tracr RNA can contain modifications. Such modifications may be at one or both ends of the crRNA and/or tracr RNA. In certain embodiments comprising an sgRNA, one or more residues at one or both ends of the sgRNA may be chemically modified, or the entire sgRNA may be chemically modified. Certain embodiments comprise a 5′ end modification. Certain embodiments comprise a 3′ end modification. In certain embodiments, one or more or all of the nucleotides in single stranded overhang of a guide RNA molecule are deoxynucleotides. The modified mRNA can contain 5′ end and/or 3′ end modifications.

### C. Regulatory Elements.

The ceDNA vectors as described herein comprising an asymmetric ITR pair or symmetric ITR pair as defined herein, can further comprise a specific combination of cis-regulatory elements. The cis-regulatory elements include, but are not limited to, a promoter, a riboswitch, an insulator, a mir-regulatable element, a post-transcriptional regulatory element, a tissue- and cell type-specific promoter and an enhancer. In some embodiments, the ITR can act as the promoter for the transgene. In some embodiments, the ceDNA vector for insertion of a transgene at a GSH locus comprises additional components to regulate expression of the transgene, for example, regulatory switches as described herein, to regulate the expression of the transgene, or a kill switch, which can kill a cell comprising the ceDNA vector. Regulatory elements, including Regulatory Switches that can be used in the present invention are more fully discussed in International application PCT/US18/49996, which is incorporated herein in its entirety by reference.

In embodiments, the second nucleotide sequence includes a regulatory sequence, and a nucleotide sequence encoding a nuclease. In certain embodiments the gene regulatory sequence is operably linked to the nucleotide sequence encoding the nuclease. In certain embodiments, the regulatory sequence is suitable for controlling the expression of the nuclease in a host cell. In certain embodiments, the regulatory sequence includes a suitable promoter sequence, being able to direct transcription of a gene operably linked to the promoter sequence, such as a nucleotide sequence encoding the nuclease(s) of the present disclosure. In certain embodiments, the second nucleotide sequence includes an intron sequence linked to the 5′ terminus of the nucleotide sequence encoding the nuclease. In certain embodiments, an enhancer sequence is provided upstream of the promoter to increase the efficacy of the promoter. In certain embodiments, the regulatory sequence includes an enhancer and a promoter, wherein the second nucleotide sequence includes an intron sequence upstream of the nucleotide sequence encoding a nuclease, wherein the intron includes one or more nuclease cleavage site(s), and wherein the promoter is operably linked to the nucleotide sequence encoding the nuclease.

The ceDNA vectors for insertion of a transgene at a GSH locus as disclosed herein which are produced synthetically, or using a cell-based production method as described herein in the Examples, can further comprise a specific combination of cis-regulatory elements such as WHP posttranscriptional regulatory element (WPRE) (e.g., SEQ ID NO: 67) and BGH polyA (SEQ ID NO: 68). Suitable expression cassettes for use in expression constructs are not limited by the packaging constraint imposed by the viral capsid.

### (i). Promoters:

It will be appreciated by one of ordinary skill in the art that promoters used in the ceDNA vectors of the invention should be tailored as appropriate for the specific sequences they are promoting. For example, a guide RNA may not require a promoter at all, since its function is to form a duplex with a specific target sequence on the native DNA to effect a recombination event. In contrast, a nuclease encoded by the ceDNA vector would benefit from a promoter so that it can be efficiently expressed from the vector—and, optionally, in a regulatable fashion.

Expression cassettes of the present invention include a promoter, which can influence overall expression levels as well as cell-specificity. For transgene expression, they can include a highly active virus-derived immediate early promoter. Expression cassettes can contain tissue-specific eukaryotic promoters to limit transgene expression to specific cell types and reduce toxic effects and immune responses resulting from unregulated, ectopic expression. In some embodiments, an expression cassette can contain a synthetic regulatory element, such as a CAG promoter (SEQ ID NO: 72). The CAG promoter comprises (i) the cytomegalovirus (CMV) early enhancer element, (ii) the promoter, the first exon and the first intron of chicken beta-actin gene, and (iii) the splice acceptor of the rabbit beta-globin gene. Alternatively, an expression cassette can contain an Alpha-1-antitrypsin (AAT) promoter (SEQ ID NO: 73 or SEQ ID NO: 74), a liver specific (LP1) promoter (SEQ ID NO: 75 or SEQ ID NO: 76), or a Human elongation factor-1 alpha (EF1a) promoter (e.g., SEQ ID NO: 77 or SEQ ID NO: 78). In some embodiments, the expression cassette includes one or more constitutive promoters, for example, a retroviral Rous sarcoma virus (RSV) LTR promoter (optionally with the RSV enhancer), or a cytomegalovirus (CMV) immediate early promoter (optionally with the CMV enhancer, e.g., SEQ ID NO: 79). Alternatively, an inducible promoter, a native promoter for a transgene, a tissue-specific promoter, or various promoters known in the art can be used.

Suitable promoters, including those described above, can be derived from viruses and can therefore be referred to as viral promoters, or they can be derived from any organism, including prokaryotic or eukaryotic organisms. Suitable promoters can be used to drive expression by any RNA polymerase (e.g., pol I, pol II, pol III). Exemplary promoters include, but are not limited to the SV40 early promoter, mouse mammary tumor virus long terminal repeat (LTR) promoter; adenovirus major late promoter (Ad MLP); a herpes simplex virus (HSV) promoter, a cytomegalovirus (CMV) promoter such as the CMV immediate early promoter region (CMVIE), a rous sarcoma virus (RSV) promoter, a human U6 small nuclear promoter (U6, e.g., SEQ ID NO: 80) (Miyagishi et al., Nature Biotechnology 20, 497-500 (2002)), an enhanced U6 promoter (e.g., Xia et al., Nucleic Acids Res. 2003 Sep. 1; 31(17)), a human H1 promoter (H1) (e.g., SEQ ID NO: 81), a CAG promoter (SEQ ID NO: 72), a human alpha 1-antitypsin (HAAT) promoter (e.g., SEQ ID NO: 82), and the like. In certain embodiments, these promoters are altered at their downstream intron containing end to include one or more nuclease cleavage sites. In certain embodiments, the DNA containing the nuclease cleavage site(s) is foreign to the promoter DNA.

In one embodiment, the promoter used is the native promoter of the gene encoding the therapeutic protein. The promoters and other regulatory sequences for the respective genes encoding the therapeutic proteins are known and have been characterized. The promoter region used may further include one or more additional regulatory sequences (e.g., native), e.g., enhancers, (e.g. SEQ ID NO: 79 and SEQ ID NO: 83), including a SV40 enhancer (SEQ ID NO: 126).

Non-limiting examples of suitable promoters for use in accordance with the present invention include the CAG promoter of, for example (SEQ ID NO: 72), the HAAT promoter (SEQ ID NO: 82), the human EF1-α promoter (SEQ ID NO: 77) or a fragment of the EF1a promoter (SEQ ID NO: 78), IE2 promoter (e.g., SEQ ID NO: 84) and the rat EF1-α promoter (SEQ ID NO: 85), or IE1 promoter fragment (SEQ ID NO: 125).

### (ii). Polyadenylation Sequences:

A sequence encoding a polyadenylation sequence can be included in the ceDNA vector for insertion of a transgene at a GSH locus to stabilize an mRNA expressed from the ceDNA vector, and to aid in nuclear export and translation. In one embodiment, the ceDNA vector does not include a polyadenylation sequence. In other embodiments, the vector includes at least 1, at least 2, at least 3, at least 4, at least 5, at least 10, at least 15, at least 20, at least 25, at least 30, at least 40, least 45, at least 50 or more adenine dinucleotides. In some embodiments, the polyadenylation sequence comprises about 43 nucleotides, about 40-50 nucleotides, about 40-55 nucleotides, about 45-50 nucleotides, about 35-50 nucleotides, or any range there between. In some embodiments, where the ceDNA vector for insertion of a transgene at a GSH locus can comprises two transgenes, e.g., in the case of controlled expression of an antibody, a ceDNA vector can comprise a nucleic acid encoding an antibody heavy chain (e.g., an exemplary heavy chain is SEQ ID NO: 57) and a nucleic acid encoding an antibody light chain (e.g., an exemplary light chain is SEQ ID NO: 58), and there can be a polyadenylation 3′ of the first transgene, and an IRES (e.g., SEQ ID NO: 190) located between the first and second transgene (e.g., between the nucleic acid encoding an antibody heavy chain and the nucleic acid encoding an antibody light chain). In such embodiments, a ceDNA vector for insertion of a transgene at a GSH locus that encodes more than one transgene (e.g., 2, or 3 or more) can comprise an IRES (internal ribosome entry site) sequence (SEQ ID NO: 190), e.g., where the IRES sequence is located 3′ of a polyadenylation sequence, such that a second transgene (e.g., antibody or antigen-binding fragment) that is located 3′ of a first transgene, is translated and expressed by the same ceDNA vector, such that the ceDNA vector can express two or more transgenes encoded by the ceDNA vector.

The expression cassettes can include a poly-adenylation sequence known in the art or a variation thereof, such as a naturally occurring sequence isolated from bovine BGHpA (e.g., SEQ ID NO: 68) or a virus SV40 pA (e.g., SEQ ID NO: 86), or a synthetic sequence (e.g., SEQ ID NO: 87). Some expression cassettes can also include SV40 late polyA signal upstream enhancer (USE) sequence. In some embodiments, the, USE can be used in combination with SV40 pA or heterologous poly-A signal.

The expression cassettes can also include a post-transcriptional element to increase the expression of a transgene. In some embodiments, Woodchuck Hepatitis Virus (WHP) posttranscriptional regulatory element (WPRE) (e.g., SEQ ID NO: 67) is used to increase the expression of a transgene. Other posttranscriptional processing elements such as the post-transcriptional element from the thymidine kinase gene of herpes simplex virus, or hepatitis B virus (HBV) can be used. Secretory sequences can be linked to the transgenes, e.g., VH-02 (SEQ ID NO: 88) and VK-A26 sequences (SEQ ID NO: 89), or IgK signal sequence (SEQ ID NO: 128), Glu secretory signal sequence (SEQ ID NO: 188) or TND secretory signal sequence (SEQ ID NO: 189).

(iii). Nuclear Localization Sequences

In some embodiments, the vector encoding an RNA guided endonuclease comprises one or more nuclear localization sequences (NLSs), for example, 1, 2, 3, 4, 5, 6, 7, 8, 9, 10, or more NLSs. In some embodiments, the one or more NLSs are located at or near the amino-terminus, at or near the carboxy-terminus, or a combination of these (e.g., one or more NLS at the amino-terminus and/or one or more NLS at the carboxy terminus). When more than one NLS is present, each can be selected independently of the others, such that a single NLS is present in more than one copy and/or in combination with one or more other NLSs present in one or more copies. Non-limiting examples of NLSs are shown in Table 10.

TABLE 19
Nuclear Localization Signals

| | | SEQ |
|---|---|---|
| | | ID |
| SOURCE | SEQUENCE | NO. |
| SV40 virus large | PKKKRKV (encoded by | 90 |
| T-antigen | CCCAAGAAGAAGAGGAAGG | |
| | TG; SEQ ID NO: 91) | |
| nucleoplasmin | KRPAATKKAGQAKKKK | 92 |
| c-myc | PAAKRVKLD | 93 |
| | RQRRNELKRSP | 94 |
| hRNPA1 M9 | NQSSNFGPMKGGNFGGRSSGP | 95 |
| | YGGGGQYFAKPRNQGGY | |
| IBB domain from | RMRIZFKNKGKDTAELRRRRV | 96 |
| importin-alpha | EVSVELRKAKKDEQILKRRNV | |
| myoma T protein | VSRKRPRP | 97 |
| | PPKKARED | 98 |
| human p53 | PQPKKKPL | 99 |
| mouse c-abl IV | SALIKKKKKMAP | 100 |
| influenza virus NS1 | DRLRR | 117 |
| | PKQKKRK | 118 |
| Hepatitis virus | RKLKKKIKKL | 119 |
| delta antigen | | |
| mouse Mx1 protein | REKKKFLKRR | 120 |
| human poly(ADP- | KRKGDEVDGVDEVAKKKSKK | 121 |
| ribose) polymerase | | |
| steroid hormone | RKCLQAGMNLEARKTKK | 122 |
| receptors (human) | | |
| glucocorticoid | | |

D. Additional Components of ceDNA Vectors

The ceDNA vectors of the present disclosure may contain nucleotides that encode other components for gene expression. For example, to select for specific gene targeting events, a protective shRNA may be embedded in a microRNA and inserted into a recombinant ceDNA vector designed to integrate site-specifically into the highly active locus, such as an albumin locus. Such embodiments may provide a system for in vivo selection and expansion of gene-modified hepatocytes in any genetic background such as described in Nygaard et al., A universal system to select gene-modified hepatocytes in vivo, Gene Therapy, Jun. 8, 2016. The ceDNA vectors of the present disclosure may contain one or more selectable markers that permit selection of transformed, transfected, transduced, or the like cells. A selectable marker is a gene the product of which provides for biocide or viral resistance, resistance to heavy metals, prototrophy to auxotrophs, NeoR, and the like. In certain embodiments, positive selection markers are incorporated into the donor sequences such as NeoR. Negative selections markers may be incorporated downstream the donor sequences, for example a nucleic acid sequence HSV-tk encoding a negative selection marker may be incorporated into a nucleic acid construct downstream the donor sequence.

In embodiments, the ceDNA vector for insertion of a transgene at a GSH locus as described herein can be used for gene editing, for example, and can comprise one or more gene editing molecules as disclosed in International Application PCT/US2018/064242, filed on Dec. 6, 2018, which is incorporated herein in its entirety by reference, and may include one or more of: a 5′ homology arm, a 3′ homology arm, a polyadenylation site upstream and proximate to the 5′ homology arm. Exemplary homology arms are 5′ and 3′ homology arms to the regions identified in Tables 1A and 1B herein.

### E. Regulatory Switches

A molecular regulatory switch is one which generates a measurable change in state in response to a signal. Such regulatory switches can be usefully combined with the ceDNA vectors described herein to control the output of expression of the transgene from the ceDNA vector. In some embodiments, the ceDNA vector for insertion of a transgene at a GSH locus as disclosed herein comprises a regulatory switch that serves to fine tune expression of the transgene. For example, it can serve as a biocontainment function of the ceDNA vector. In some embodiments, the switch is an “ON/OFF” switch that is designed to start or stop (i.e., shut down) expression of the gene of interest in the ceDNA in a controllable and regulatable fashion. In some embodiments, the switch can include a “kill switch” that can instruct the cell comprising the ceDNA vector to undergo cell programmed death once the switch is activated. Exemplary regulatory switches encompassed for use in a ceDNA vector for insertion of a transgene at a GSH locus can be used to regulate the expression of a transgene, and are more fully discussed in International application PCT/US18/49996, which is incorporated herein in its entirety by reference

### (i) Binary Regulatory Switches

In some embodiments, the ceDNA vector for insertion of a transgene at a GSH locus comprises a regulatory switch that can serve to controllably modulate expression of the transgene. For example, the expression cassette located between the ITRs of the ceDNA vector for insertion of a transgene at a GSH locus may additionally comprise a regulatory region, e.g., a promoter, cis-element, repressor, enhancer etc., that is operatively linked to the gene of interest, where the regulatory region is regulated by one or more cofactors or exogenous agents. By way of example only, regulatory regions can be modulated by small molecule switches or inducible or repressible promoters. Nonlimiting examples of inducible promoters are hormone-inducible or metal-inducible promoters. Other exemplary inducible promoters/enhancer elements include, but are not limited to, an RU486-inducible promoter, an ecdysone-inducible promoter, a rapamycin-inducible promoter, and a metallothionein promoter.

### (ii) Small Molecule Regulatory Switches

A variety of art-known small-molecule based regulatory switches are known in the art and can be combined with the ceDNA vectors disclosed herein to form a regulatory-switch controlled ceDNA vector. In some embodiments, the regulatory switch can be selected from any one or a combination of: an orthogonal ligand/nuclear receptor pair, for example retinoid receptor variant/LG335 and GRQCIMFI, along with an artificial promoter controlling expression of the operatively linked transgene, such as that as disclosed in Taylor, et al. BMC Biotechnology 10 (2010): 15; engineered steroid receptors, e.g., modified progesterone receptor with a C-terminal truncation that cannot bind progesterone but binds RU486 (mifepristone) (U.S. Pat. No. 5,364,791); an ecdysone receptor from Drosophila and their ecdysteroid ligands (Saez, et al., PNAS, 97(26)(2000), 14512-14517; or a switch controlled by the antibiotic trimethoprim (TMP), as disclosed in Sando R 3^{rd}; Nat Methods. 2013, 10(11):1085-8. In some embodiments, the regulatory switch to control the transgene or expressed by the ceDNA vector for insertion of a transgene at a GSH locus is a pro-drug activation switch, such as that disclosed in U.S. Pat. Nos. 8,771,679, and 6,339,070.

### “Passcode” Regulatory Switches

In some embodiments the regulatory switch can be a “passcode switch” or “passcode circuit”. Passcode switches allow fine tuning of the control of the expression of the transgene from the ceDNA vector for insertion of a transgene at a GSH locus when specific conditions occur—that is, a combination of conditions need to be present for transgene expression and/or repression to occur. For example, for expression of a transgene to occur at least conditions A and B must occur. A passcode regulatory switch can be any number of conditions, e.g., at least 2, or at least 3, or at least 4, or at least 5, or at least 6 or at least 7 or more conditions to be present for transgene expression to occur. In some embodiments, at least 2 conditions (e.g., A, B conditions) need to occur, and in some embodiments, at least 3 conditions need to occur (e.g., A, B and C, or A, B and D). By way of an example only, for gene expression from a ceDNA to occur that has a passcode “ABC” regulatory switch, conditions A, B and C must be present. Conditions A, B and C could be as follows; condition A is the presence of a condition or disease, condition B is a hormonal response, and condition C is a response to the transgene expression. For example, if the transgene edits a defective EPO gene, Condition A is the presence of Chronic Kidney Disease (CKD), Condition B occurs if the subject has hypoxic conditions in the kidney, Condition C is that Erythropoietin-producing cells (EPC) recruitment in the kidney is impaired; or alternatively, HIF-2 activation is impaired. Once the oxygen levels increase or the desired level of EPO is reached, the transgene turns off again until 3 conditions occur, turning it back on.

In some embodiments, a passcode regulatory switch or “Passcode circuit” encompassed for use in the ceDNA vector for insertion of a transgene at a GSH locus comprises hybrid transcription factors (TFs) to expand the range and complexity of environmental signals used to define biocontainment conditions. As opposed to a deadman switch which triggers cell death in the presence of a predetermined condition, the “passcode circuit” allows cell survival or transgene expression in the presence of a particular “passcode”, and can be easily reprogrammed to allow transgene expression and/or cell survival only when the predetermined environmental condition or passcode is present.

Any and all combinations of regulatory switches disclosed herein, e.g., small molecule switches, nucleic acid-based switches, small molecule-nucleic acid hybrid switches, post-transcriptional transgene regulation switches, post-translational regulation, radiation-controlled switches, hypoxia-mediated switches and other regulatory switches known by persons of ordinary skill in the art as disclosed herein can be used in a passcode regulatory switch as disclosed herein. Regulatory switches encompassed for use are also discussed in the review article Kis et al., J R Soc Interface. 12: 20141000 (2015), and summarized in Table 1 of Kis. In some embodiments, a regulatory switch for use in a passcode system can be selected from any or a combination of the switches in Table 11 of International Patent Application PCT/US18/49996, filed Sep. 7, 2018, which is incorporated herein in its entirety.

### (iv). Nucleic Acid-Based Regulatory Switches to Control Transgene Expression

In some embodiments, the regulatory switch to control the transgene expressed by the ceDNA is based on a nucleic-acid based control mechanism. Exemplary nucleic acid control mechanisms are known in the art and are envisioned for use. For example, such mechanisms include riboswitches, such as those disclosed in, e.g., US2009/0305253, US2008/0269258, US2017/0204477, WO2018026762A1, U.S. Pat. No. 9,222,093 and EP application EP288071, and also disclosed in the review by Villa J K et al., Microbiol Spectr. 2018 May; 6(3). Also included are metabolite-responsive transcription biosensors, such as those disclosed in WO2018/075486 and WO2017/147585. Other art-known mechanisms envisioned for use include silencing of the transgene with an siRNA or RNAi molecule (e.g., miR, shRNA). For example, the ceDNA vector for insertion of a transgene at a GSH locus can comprise a regulatory switch that encodes a RNAi molecule that is complementary to the transgene expressed by the ceDNA vector. When such RNAi is expressed even if the transgene is expressed by the ceDNA vector, it will be silenced by the complementary RNAi molecule, and when the RNAi is not expressed when the transgene is expressed by the ceDNA vector the transgene is not silenced by the RNAi.

In some embodiments, the regulatory switch is a tissue-specific self-inactivating regulatory switch, for example as disclosed in US2002/0022018, whereby the regulatory switch deliberately switches transgene expression off at a site where transgene expression might otherwise be disadvantageous. In some embodiments, the regulatory switch is a recombinase reversible gene expression system, for example as disclosed in US2014/0127162 and U.S. Pat. No. 8,324,436.

### (v). Post-Transcriptional and Post-Translational Regulatory Switches.

In some embodiments, the regulatory switch to control the transgene or gene of interest expressed by the ceDNA vector for insertion of a transgene at a GSH locus is a post-transcriptional modification system. For example, such a regulatory switch can be an aptazyme riboswitch that is sensitive to tetracycline or theophylline, as disclosed in US2018/0119156, GB201107768, WO2001/064956A3, EP Patent 2707487 and Beilstein et al., ACS Synth. Biol., 2015, 4 (5), pp 526-534; Zhong et al., Elife. 2016 Nov. 2; 5. pii: e18858. In some embodiments, it is envisioned that a person of ordinary skill in the art could encode both the transgene and an inhibitory siRNA which contains a ligand sensitive (OFF-switch) aptamer, the net result being a ligand sensitive ON-switch.

### (vi). Other Exemplary Regulatory Switches

Any known regulatory switch can be used in the ceDNA vector to control the gene expression of the transgene expressed by the ceDNA vector, including those triggered by environmental changes. Additional examples include, but are not limited to; the BOC method of Suzuki et al., Scientific Reports 8; 10051 (2018); genetic code expansion and a non-physiologic amino acid; radiation-controlled or ultra-sound controlled on/off switches (see, e.g., Scott S et al., Gene Ther. 2000 July; 7(13):1121-5; U.S. Pat. Nos. 5,612,318; 5,571,797; 5,770,581; 5,817,636; and WO1999/025385A1. In some embodiments, the regulatory switch is controlled by an implantable system, e.g., as disclosed in U.S. Pat. No. 7,840,263; US2007/0190028A1 where gene expression is controlled by one or more forms of energy, including electromagnetic energy, that activates promoters operatively linked to the transgene in the ceDNA vector.

In some embodiments, a regulatory switch envisioned for use in the ceDNA vector for insertion of a transgene at a GSH locus is a hypoxia-mediated or stress-activated switch, e.g., such as those disclosed in WO1999060142A2, U.S. Pat. Nos. 5,834,306; 6,218,179; 6,709,858; US2015/0322410; Greco et al., (2004) Targeted Cancer Therapies 9, S368, as well as FROG, TOAD and NRSE elements and conditionally inducible silence elements, including hypoxia response elements (HREs), inflammatory response elements (IREs) and shear-stress activated elements (SSAEs), e.g, as disclosed in U.S. Pat. No. 9,394,526. Such an embodiment is useful for turning on expression of the transgene from the ceDNA vector for insertion of a transgene at a GSH locus after ischemia or in ischemic tissues, and/or tumors.

### (iv). Kill Switches

Other embodiments of the invention relate to a ceDNA vector for insertion of a transgene at a GSH locus comprising a kill switch. A kill switch as disclosed herein enables a cell comprising the ceDNA vector to be killed or undergo programmed cell death as a means to permanently remove an introduced ceDNA vector from the subject's system. It will be appreciated by one of ordinary skill in the art that use of kill switches in the ceDNA vectors of the invention would be typically coupled with targeting of the ceDNA vector to a limited number of cells that the subject can acceptably lose or to a cell type where apoptosis is desirable (e.g., cancer cells). In all aspects, a “kill switch” as disclosed herein is designed to provide rapid and robust cell killing of the cell comprising the ceDNA vector in the absence of an input survival signal or other specified condition. Stated another way, a kill switch encoded by a ceDNA vector herein can restrict cell survival of a cell comprising a ceDNA vector to an environment defined by specific input signals. Such kill switches serve as a biological biocontainment function should it be desirable to remove the ceDNA vector from a subject or to ensure that it will not express the encoded transgene.

VI. Detailed Method of Production of a ceDNA Vector

### A. Production in General

Certain methods for the production of a ceDNA vector for insertion of a transgene at a GSH locus comprising an asymmetrical ITR pair or symmetrical ITR pair as defined herein is described in section IV of International application PCT/US18/49996 filed Sep. 7, 2018, which is incorporated herein in its entirety by reference. In some embodiments, a ceDNA vector for insertion of a transgene at a GSH locus for use in the methods and compositions as disclosed herein can be produced using insect cells, as described herein. In alternative embodiments, a for use in the methods and compositions as disclosed herein can be produced synthetically, and in some embodiments, in a cell-free method, as disclosed on International Application PCT/US19/14122, filed Jan. 18, 2019, which is incorporated herein in its entirety by reference.

As described herein, in one embodiment, a ceDNA vector for insertion of a transgene at a GSH locus can be obtained, for example, by the process comprising the steps of: a) incubating a population of host cells (e.g. insect cells) harboring the polynucleotide expression construct template (e.g., a ceDNA-plasmid, a ceDNA-Bacmid, and/or a ceDNA-baculovirus), which is devoid of viral capsid coding sequences, in the presence of a Rep protein under conditions effective and for a time sufficient to induce production of the ceDNA vector within the host cells, and wherein the host cells do not comprise viral capsid coding sequences; and b) harvesting and isolating the ceDNA vector from the host cells. The presence of Rep protein induces replication of the vector polynucleotide with a modified ITR to produce the ceDNA vector in a host cell. However, no viral particles (e.g. AAV virions) are expressed. Thus, there is no size limitation such as that naturally imposed in AAV or other viral-based vectors.

The presence of the ceDNA vector isolated from the host cells can be confirmed by digesting DNA isolated from the host cell with a restriction enzyme having a single recognition site on the ceDNA vector and analyzing the digested DNA material on a non-denaturing gel to confirm the presence of characteristic bands of linear and continuous DNA as compared to linear and non-continuous DNA.

In yet another aspect, the invention provides for use of host cell lines that have stably integrated the DNA vector polynucleotide expression template (ceDNA template) into their own genome in production of the non-viral DNA vector, e.g. as described in Lee, L. et al. (2013) Plos One 8(8): e69879. Preferably, Rep is added to host cells at an MOI of about 3. When the host cell line is a mammalian cell line, e.g., HEK293 cells, the cell lines can have polynucleotide vector template stably integrated, and a second vector such as herpes virus can be used to introduce Rep protein into cells, allowing for the excision and amplification of ceDNA in the presence of Rep and helper virus.

In one embodiment, the host cells used to make the ceDNA vectors described herein are insect cells, and baculovirus is used to deliver both the polynucleotide that encodes Rep protein and the non-viral DNA vector polynucleotide expression construct template for ceDNA, e.g., as described in FIGS. 4A-4C and Example 1. In some embodiments, the host cell is engineered to express Rep protein.

The ceDNA vector is then harvested and isolated from the host cells. The time for harvesting and collecting ceDNA vectors described herein from the cells can be selected and optimized to achieve a high-yield production of the ceDNA vectors. For example, the harvest time can be selected in view of cell viability, cell morphology, cell growth, etc. In one embodiment, cells are grown under sufficient conditions and harvested a sufficient time after baculoviral infection to produce ceDNA vectors but before a majority of cells start to die because of the baculoviral toxicity. The DNA vectors can be isolated using plasmid purification kits such as Qiagen Endo-Free Plasmid kits. Other methods developed for plasmid isolation can be also adapted for DNA vectors. Generally, any nucleic acid purification methods can be adopted.

The DNA vectors can be purified by any means known to those of skill in the art for purification of DNA. In one embodiment, ceDNA vectors are purified as DNA molecules. In another embodiment, the ceDNA vectors are purified as exosomes or microparticles.

The presence of the ceDNA vector can be confirmed by digesting the vector DNA isolated from the cells with a restriction enzyme having a single recognition site on the DNA vector and analyzing both digested and undigested DNA material using gel electrophoresis to confirm the presence of characteristic bands of linear and continuous DNA as compared to linear and non-continuous DNA. FIG. 4C and FIG. 4D illustrate one embodiment for identifying the presence of the closed ended ceDNA vectors produced by the processes herein.

B. ceDNA Plasmid

A ceDNA-plasmid is a plasmid used for later production of a ceDNA vector. In some embodiments, a ceDNA-plasmid can be constructed using known techniques to provide at least the following as operatively linked components in the direction of transcription: (1) a modified 5′ ITR sequence; (2) an expression cassette containing a cis-regulatory element, for example, a promoter, inducible promoter, regulatory switch, enhancers and the like; and (3) a modified 3′ ITR sequence, where the 3′ ITR sequence is symmetric relative to the 5′ ITR sequence. In some embodiments, the expression cassette flanked by the ITRs comprises a cloning site for introducing an exogenous sequence. The expression cassette replaces the rep and cap coding regions of the AAV genomes.

In one aspect, a ceDNA vector for insertion of a transgene at a GSH locus is obtained from a plasmid, referred to herein as a “ceDNA-plasmid” encoding in this order: a first adeno-associated virus (AAV) inverted terminal repeat (ITR), an expression cassette comprising a transgene, and a mutated or modified AAV ITR, wherein said ceDNA-plasmid is devoid of AAV capsid protein coding sequences. In alternative embodiments, the ceDNA-plasmid encodes in this order: a first (or 5′) modified or mutated AAV ITR, an expression cassette comprising a transgene, and a second (or 3′) modified AAV ITR, wherein said ceDNA-plasmid is devoid of AAV capsid protein coding sequences, and wherein the 5′ and 3′ ITRs are symmetric relative to each other. In alternative embodiments, the ceDNA-plasmid encodes in this order: a first (or 5′) modified or mutated AAV ITR, an expression cassette comprising a transgene, and a second (or 3′) mutated or modified AAV ITR, wherein said ceDNA-plasmid is devoid of AAV capsid protein coding sequences, and wherein the 5′ and 3′ modified ITRs are have the same modifications (i.e., they are inverse complement or symmetric relative to each other).

In a further embodiment, the ceDNA-plasmid system is devoid of viral capsid protein coding sequences (i.e. it is devoid of AAV capsid genes but also of capsid genes of other viruses). In addition, in a particular embodiment, the ceDNA-plasmid is also devoid of AAV Rep protein coding sequences. Accordingly, in a preferred embodiment, ceDNA-plasmid is devoid of functional AAV cap and AAV rep genes GG-3′ for AAV2) plus a variable palindromic sequence allowing for hairpin formation.

A ceDNA-plasmid of the present invention can be generated using natural nucleotide sequences of the genomes of any AAV serotypes well known in the art. In one embodiment, the ceDNA-plasmid backbone is derived from the AAV1, AAV2, AAV3, AAV4, AAV5, AAV 5, AAV7, AAV8, AAV9, AAV10, AAV 11, AAV12, AAVrh8, AAVrh10, AAV-DJ, and AAV-DJ8 genome. E.g., NCBI: NC 002077; NC 001401; NC001729; NC001829; NC006152; NC 006260; NC 006261; Kotin and Smith, The Springer Index of Viruses, available at the URL maintained by Springer (at www web address: oesys.springer.de/viruses/database/mkchapter.asp?virID=42.04.)(note—references to a URL or database refer to the contents of the URL or database as of the effective filing date of this application) In a particular embodiment, the ceDNA-plasmid backbone is derived from the AAV2 genome. In another particular embodiment, the ceDNA-plasmid backbone is a synthetic backbone genetically engineered to include at its 5′ and 3′ ITRs derived from one of these AAV genomes.

A ceDNA-plasmid can optionally include a selectable or selection marker for use in the establishment of a ceDNA vector-producing cell line. In one embodiment, the selection marker can be inserted downstream (i.e., 3′) of the 3′ ITR sequence. In another embodiment, the selection marker can be inserted upstream (i.e., 5′) of the 5′ ITR sequence. Appropriate selection markers include, for example, those that confer drug resistance. Selection markers can be, for example, a blasticidin S-resistance gene, kanamycin, geneticin, and the like. In a preferred embodiment, the drug selection marker is a blasticidin S-resistance gene.

An Exemplary ceDNA (e.g., rAAV0) is produced from an rAAV plasmid. A method for the production of a rAAV vector, can comprise: (a) providing a host cell with a rAAV plasmid as described above, wherein both the host cell and the plasmid are devoid of capsid protein encoding genes, (b) culturing the host cell under conditions allowing production of an ceDNA genome, and (c) harvesting the cells and isolating the AAV genome produced from said cells.

C. Exemplary Method of Making the ceDNA Vectors from ceDNA Plasmids

Methods for making capsid-less ceDNA vectors are also provided herein, notably a method with a sufficiently high yield to provide sufficient vector for in vivo experiments.

In some embodiments, a method for the production of a ceDNA vector for insertion of a transgene at a GSH locus comprises the steps of: (1) introducing the nucleic acid construct comprising an expression cassette and two symmetric ITR sequences into a host cell (e.g., Sf9 cells), (2) optionally, establishing a clonal cell line, for example, by using a selection marker present on the plasmid, (3) introducing a Rep coding gene (either by transfection or infection with a baculovirus carrying said gene) into said insect cell, and (4) harvesting the cell and purifying the ceDNA vector. The nucleic acid construct comprising an expression cassette and two ITR sequences described above for the production of ceDNA vector for insertion of a transgene at a GSH locus can be in the form of a ceDNA plasmid, or Bacmid or Baculovirus generated with the ceDNA plasmid as described below. The nucleic acid construct can be introduced into a host cell by transfection, viral transduction, stable integration, or other methods known in the art.

### D. Cell Lines:

Host cell lines used in the production of a ceDNA vector for insertion of a transgene at a GSH locus can include insect cell lines derived from Spodoptera frugiperda, such as Sf9 Sf21, or Trichoplusia ni cell, or other invertebrate, vertebrate, or other eukaryotic cell lines including mammalian cells. Other cell lines known to an ordinarily skilled artisan can also be used, such as HEK293, Huh-7, HeLa, HepG2, Hep1A, 911, CHO, COS, MeWo, NIH3T3, A549, HT1 180, monocytes, and mature and immature dendritic cells. Host cell lines can be transfected for stable expression of the ceDNA-plasmid for high yield ceDNA vector production.

CeDNA-plasmids can be introduced into Sf9 cells by transient transfection using reagents (e.g., liposomal, calcium phosphate) or physical means (e.g., electroporation) known in the art. Alternatively, stable Sf9 cell lines which have stably integrated the ceDNA-plasmid into their genomes can be established. Such stable cell lines can be established by incorporating a selection marker into the ceDNA-plasmid as described above. If the ceDNA-plasmid used to transfect the cell line includes a selection marker, such as an antibiotic, cells that have been transfected with the ceDNA-plasmid and integrated the ceDNA-plasmid DNA into their genome can be selected for by addition of the antibiotic to the cell growth media. Resistant clones of the cells can then be isolated by single-cell dilution or colony transfer techniques and propagated.

E. Isolating and Purifying ceDNA Vectors:

Examples of the process for obtaining and isolating ceDNA vectors are described in FIGS. 4A-4E and the specific examples below. ceDNA-vectors disclosed herein can be obtained from a producer cell expressing AAV Rep protein(s), further transformed with a ceDNA-plasmid, ceDNA-bacmid, or ceDNA-baculovirus. Plasmids useful for the production of ceDNA vectors include plasmids incorporating one or more Rep protein(s) and plasmids used to obtain a ceDNA vector. Exemplary plasmids for production of ceDNA vector to for insertion of a transgene at a GSH locus as disclosed herein is a modified plasmid to the plasmid as shown in FIG. 6B of International application PCT/US2018/064242, filed Dec. 6, 2018, which is incorporated herein in its entirety. A ceDNA plasmid for production of a ceDNA vector for insertion of a transgene at a GSH locus is disclosed in FIG. 6A and is SEQ ID NO: 56 of International Application PCT/US19/18016 filed on Feb. 14, 2019, which discloses an exemplary ceDNA plasmid for production of aducanmab, but can be modified to include a HA-L and HA-R flanking the nucleic acid sequences (and regulatory sequences), encoding the aducanmab antibody.

In one aspect, a polynucleotide encodes the AAV Rep protein (Rep 78 or Rep68) is delivered to a producer cell in a plasmid (Rep-plasmid), a bacmid (Rep-bacmid), or a baculovirus (Rep-baculovirus). The Rep-plasmid, Rep-bacmid, and Rep-baculovirus can be generated by methods described above.

Methods to produce a ceDNA-vector, which is an exemplary ceDNA vector, are described herein. Expression constructs used for generating a ceDNA vectors of the present invention can be a plasmid (e.g., ceDNA-plasmids), a Bacmid (e.g., ceDNA-bacmid), and/or a baculovirus (e.g., ceDNA-baculovirus). By way of an example only, a ceDNA-vector can be generated from the cells co-infected with ceDNA-baculovirus and Rep-baculovirus. Rep proteins produced from the Rep-baculovirus can replicate the ceDNA-baculovirus to generate ceDNA-vectors. Alternatively, ceDNA vectors can be generated from the cells stably transfected with a construct comprising a sequence encoding the AAV Rep protein (Rep78/52) delivered in Rep-plasmids, Rep-bacmids, or Rep-baculovirus. CeDNA-Baculovirus can be transiently transfected to the cells, be replicated by Rep protein and produce ceDNA vectors.

The bacmid (e.g., ceDNA-bacmid) can be transfected into a permissive insect cells such as Sf9, Sf21, Tni (Trichoplusia ni) cell, High Five cell, and generate ceDNA-baculovirus, which is a recombinant baculovirus including the sequences comprising the symmetric ITRs and the expression cassette. ceDNA-baculovirus can be again infected into the insect cells to obtain a next generation of the recombinant baculovirus. Optionally, the step can be repeated once or multiple times to produce the recombinant baculovirus in a larger quantity.

The time for harvesting and collecting ceDNA vectors described herein from the cells can be selected and optimized to achieve a high-yield production of the ceDNA vectors. For example, the harvest time can be selected in view of cell viability, cell morphology, cell growth, etc. Usually, cells can be harvested after sufficient time after baculoviral infection to produce ceDNA vectors (e.g., ceDNA vectors) but before majority of cells start to die because of the viral toxicity. The ceDNA-vectors can be isolated from the Sf9 cells using plasmid purification kits such as Qiagen ENDO-FREE PLASMID® kits. Other methods developed for plasmid isolation can be also adapted for ceDNA vectors. Generally, any art-known nucleic acid purification methods can be adopted, as well as commercially available DNA extraction kits.

Alternatively, purification can be implemented by subjecting a cell pellet to an alkaline lysis process, centrifuging the resulting lysate and performing chromatographic separation. As one nonlimiting example, the process can be performed by loading the supernatant on an ion exchange column (e.g. SARTOBIND Q®) which retains nucleic acids, and then eluting (e.g. with a 1.2 M NaCl solution) and performing a further chromatographic purification on a gel filtration column (e.g. 6 fast flow GE). The capsid-free AAV vector is then recovered by, e.g., precipitation.

In some embodiments, ceDNA vectors can also be purified in the form of exosomes, or microparticles. It is known in the art that many cell types release not only soluble proteins, but also complex protein/nucleic acid cargoes via membrane microvesicle shedding (Cocucci et al, 2009; EP 10306226.1) Such vesicles include microvesicles (also referred to as microparticles) and exosomes (also referred to as nanovesicles), both of which comprise proteins and RNA as cargo. Microvesicles are generated from the direct budding of the plasma membrane, and exosomes are released into the extracellular environment upon fusion of multivesicular endosomes with the plasma membrane. Thus, ceDNA vector-containing microvesicles and/or exosomes can be isolated from cells that have been transduced with the ceDNA-plasmid or a bacmid or baculovirus generated with the ceDNA-plasmid.

Microvesicles can be isolated by subjecting culture medium to filtration or ultracentrifugation at 20,000×g, and exosomes at 100,000×g. The optimal duration of ultracentrifugation can be experimentally-determined and will depend on the particular cell type from which the vesicles are isolated. Preferably, the culture medium is first cleared by low-speed centrifugation (e.g., at 2000× g for 5-20 minutes) and subjected to spin concentration using, e.g., an AMICON® spin column (Millipore, Watford, UK). Microvesicles and exosomes can be further purified via FACS or MACS by using specific antibodies that recognize specific surface antigens present on the microvesicles and exosomes. Other microvesicle and exosome purification methods include, but are not limited to, immunoprecipitation, affinity chromatography, filtration, and magnetic beads coated with specific antibodies or aptamers. Upon purification, vesicles are washed with, e.g., phosphate-buffered saline. One advantage of using microvesicles or exosome to deliver ceDNA-containing vesicles is that these vesicles can be targeted to various cell types by including on their membranes proteins recognized by specific receptors on the respective cell types. (See also EP 10306226)

Another aspect of the invention herein relates to methods of purifying ceDNA vectors from host cell lines that have stably integrated a ceDNA construct into their own genome. In one embodiment, ceDNA vectors are purified as DNA molecules. In another embodiment, the ceDNA vectors are purified as exosomes or microparticles.

FIG. 5 of International application PCT/US18/49996 shows a gel confirming the production of ceDNA from multiple ceDNA-plasmid constructs using the method described in the Examples. The ceDNA is confirmed by a characteristic band pattern in the gel, as discussed with respect to FIG. 4D in the Examples.

### VII. Pharmaceutical Compositions

In another aspect, pharmaceutical compositions are provided. The pharmaceutical composition comprises a closed-ended DNA vector, e.g., ceDNA vector for insertion of a transgene at a GSH locus produced using the synthetic process as described herein and a pharmaceutically acceptable carrier or diluent.

The ceDNA vectors as disclosed herein can be incorporated into pharmaceutical compositions suitable for administration to a subject for in vivo delivery to cells, tissues, or organs of the subject. Typically, the pharmaceutical composition comprises a ceDNA-vector as disclosed herein and a pharmaceutically acceptable carrier. For example, the ceDNA vectors described herein can be incorporated into a pharmaceutical composition suitable for a desired route of therapeutic administration (e.g., parenteral administration). Passive tissue transduction via high pressure intravenous or intra-arterial infusion, as well as intracellular injection, such as intranuclear microinjection or intracytoplasmic injection, are also contemplated. Pharmaceutical compositions for therapeutic purposes can be formulated as a solution, microemulsion, dispersion, liposomes, or other ordered structure suitable to high ceDNA vector concentration. Sterile injectable solutions can be prepared by incorporating the ceDNA vector compound in the required amount in an appropriate buffer with one or a combination of ingredients enumerated above, as required, followed by filtered sterilization including a ceDNA vector can be formulated to deliver a transgene in the nucleic acid to the cells of a recipient, resulting in the therapeutic expression of the transgene or donor sequence therein. The composition can also include a pharmaceutically acceptable carrier.

Pharmaceutically active compositions comprising a ceDNA vector for insertion of a transgene at a GSH locus can be formulated to deliver a transgene for various purposes to the cell, e.g., cells of a subject.

The ceDNA vectors disclosed herein can be incorporated into pharmaceutical compositions suitable for administration to a subject for in vivo delivery to cells, tissues, or organs of the subject. Typically, the pharmaceutical composition comprises the DNA-vectors disclosed herein and a pharmaceutically acceptable carrier. For example, the ceDNA vectors of the invention can be incorporated into a pharmaceutical composition suitable for a desired route of therapeutic administration (e.g., parenteral administration). Passive tissue transduction via high pressure intravenous or intraarterial infusion, as well as intracellular injection, such as intranuclear microinjection or intracytoplasmic injection, are also contemplated. Pharmaceutical compositions for therapeutic purposes can be formulated as a solution, microemulsion, dispersion, liposomes, or other ordered structure suitable to high ceDNA vector concentration. Sterile injectable solutions can be prepared by incorporating the ceDNA vector compound in the required amount in an appropriate buffer with one or a combination of ingredients enumerated above, as required, followed by filtered sterilization.

Pharmaceutically active compositions comprising a ceDNA vector can be formulated to deliver a transgene in the nucleic acid to the cells of a recipient, resulting in the therapeutic expression of the transgene therein. The composition can also optionally include a pharmaceutically acceptable carrier and/or excipient.

The compositions and vectors provided herein can be used to deliver a transgene for various purposes. In some embodiments, the transgene encodes a protein or functional RNA that is intended to be used for research purposes, e.g., to create a somatic transgenic animal model harboring the transgene, e.g., to study the function of the transgene product. In another example, the transgene encodes a protein or functional RNA that is intended to be used to create an animal model of disease. In some embodiments, the transgene encodes one or more peptides, polypeptides, or proteins, which are useful for the treatment or prevention of disease states in a mammalian subject. The transgene can be transferred (e.g., expressed in) to a patient in a sufficient amount to treat a disease associated with reduced expression, lack of expression or dysfunction of the gene. In some embodiments, the transgene is a gene editing molecule (e.g., nuclease). In certain embodiments, the nuclease is a CRISPR-associated nuclease (Cas nuclease).

Pharmaceutical compositions for therapeutic purposes typically must be sterile and stable under the conditions of manufacture and storage. Sterile injectable solutions can be prepared by incorporating the ceDNA vector compound in the required amount in an appropriate buffer with one or a combination of ingredients enumerated above, as required, followed by filtered sterilization.

In certain circumstances, it will be desirable to deliver a ceDNA composition or vector as disclosed herein in suitably formulated pharmaceutical compositions disclosed herein either subcutaneously, intraopancreatically, intranasally, parenterally, intravenously, intramuscularly, intrathecally, systemic administration, or orally, intraperitoneally, or by inhalation.

It is specifically contemplated herein that the compositions described herein comprise a ceDNA vector for insertion of a transgene at a GSH locus at a given dose that is determined by the dose-response relationship of the ceDNA vector, for example, a “unit dose” that, upon administration, can be reliably expected to produce a desired effect or level of expression of the genetic medicine in a typical subject.

Pharmaceutical compositions for therapeutic purposes typically must be sterile and stable under the conditions of manufacture and storage. The composition can be formulated as a solution, microemulsion, dispersion, liposomes, or other ordered structure suitable to high ceDNA vector concentration. Sterile injectable solutions can be prepared by incorporating the ceDNA vector compound in the required amount in an appropriate buffer with one or a combination of ingredients enumerated above, as required, followed by filtered sterilization.

A ceDNA vector for insertion of a transgene at a GSH locus as disclosed herein can be incorporated into a pharmaceutical composition suitable for topical, systemic, intra-amniotic, intrathecal, intracranial, intra-arterial, intravenous, intralymphatic, intraperitoneal, subcutaneous, tracheal, intra-tissue (e.g., intramuscular, intracardiac, intrahepatic, intrarenal, intracerebral), intrathecal, intravesical, conjunctival (e.g., extra-orbital, intraorbital, retroorbital, intraretinal, subretinal, choroidal, sub-choroidal, intrastromal, intracameral and intravitreal), intracochlear, and mucosal (e.g., oral, rectal, nasal) administration. Passive tissue transduction via high pressure intravenous or intraarterial infusion, as well as intracellular injection, such as intranuclear microinjection or intracytoplasmic injection, are also contemplated.

In some aspects, the methods provided herein comprise delivering one or more ceDNA vectors as disclosed herein to a host cell. Also provided herein are cells produced by such methods, and organisms (such as animals, plants, or fungi) comprising or produced from such cells. Methods of delivery of nucleic acids can include lipofection, nucleofection, microinjection, biolistics, liposomes, immunoliposomes, polycation or lipid:nucleic acid conjugates, naked DNA, and agent-enhanced uptake of DNA. Lipofection is described in e.g., U.S. Pat. Nos. 5,049,386, 4,946,787; and 4,897,355) and lipofection reagents are sold commercially (e.g., Transfectam™ and Lipofectin™). Delivery can be to cells (e.g., in vitro or ex vivo administration) or target tissues (e.g., in vivo administration).

Various techniques and methods are known in the art for delivering nucleic acids to cells. For example, nucleic acids, such as ceDNA can be formulated into lipid nanoparticles (LNPs), lipidoids, liposomes, lipid nanoparticles, lipoplexes, or core-shell nanoparticles. Typically, LNPs are composed of nucleic acid (e.g., ceDNA) molecules, one or more ionizable or cationic lipids (or salts thereof), one or more non-ionic or neutral lipids (e.g., a phospholipid), a molecule that prevents aggregation (e.g., PEG or a PEG-lipid conjugate), and optionally a sterol (e.g., cholesterol).

Another method for delivering nucleic acids, such as ceDNA to a cell is by conjugating the nucleic acid with a ligand that is internalized by the cell. For example, the ligand can bind a receptor on the cell surface and internalized via endocytosis. The ligand can be covalently linked to a nucleotide in the nucleic acid. Exemplary conjugates for delivering nucleic acids into a cell are described, example, in WO2015/006740, WO2014/025805, WO2012/037254, WO2009/082606, WO2009/073809, WO2009/018332, WO2006/112872, WO2004/090108, WO2004/091515 and WO2017/177326.

Nucleic acids, such as ceDNA, can also be delivered to a cell by transfection. Useful transfection methods include, but are not limited to, lipid-mediated transfection, cationic polymer-mediated transfection, or calcium phosphate precipitation. Transfection reagents are well known in the art and include, but are not limited to, TurboFect Transfection Reagent (Thermo Fisher Scientific), Pro-Ject Reagent (Thermo Fisher Scientific), TRANSPASS™ P Protein Transfection Reagent (New England Biolabs), CHARIOT™ Protein Delivery Reagent (Active Motif), PROTEOJUICE™ Protein Transfection Reagent (EMD Millipore), 293fectin, LIPOFECTAMINE™ 2000, LIPOFECTAMINE™ 3000 (Thermo Fisher Scientific), LIPOFECTAMINE™ (Thermo Fisher Scientific), LIPOFECTIN™ (Thermo Fisher Scientific), DMRIE-C, CELLFECTIN™ (Thermo Fisher Scientific), OLIGOFECTAMINE™ (Thermo Fisher Scientific), LIPOFECTACE™, FUGENE™ (Roche, Basel, Switzerland), FUGENE™ HD (Roche), TRANSFECTAM™ (Transfectam, Promega, Madison, Wis.), TFX-10™ (Promega), TFX-20™ (Promega), TFX-50™ (Promega), TRANSFECTIN™ (BioRad, Hercules, Calif.), SILENTFECT™ (Bio-Rad), Effectene™ (Qiagen, Valencia, Calif.), DC-chol (Avanti Polar Lipids), GENEPORTER™ (Gene Therapy Systems, San Diego, Calif.), DHARMAFECT 1™ (Dharmacon, Lafayette, Colo.), DHARMAFECT 2™ (Dharmacon), DHARMAFECT 3™ (Dharmacon), DHARMAFECT4™ (Dharmacon), ESCORT™ III (Sigma, St. Louis, Mo.), and ESCORT™ IV (Sigma Chemical Co.). Nucleic acids, such as ceDNA, can also be delivered to a cell via microfluidics methods known to those of skill in the art.

ceDNA vectors as described herein can also be administered directly to an organism for transduction of cells in vivo. Administration is by any of the routes normally used for introducing a molecule into ultimate contact with blood or tissue cells including, but not limited to, injection, infusion, topical application and electroporation. Suitable methods of administering such nucleic acids are available and well known to those of skill in the art, and, although more than one route can be used to administer a particular composition, a particular route can often provide a more immediate and more effective reaction than another route.

Methods for introduction of a nucleic acid vector ceDNA vector for insertion of a transgene at a GSH locus as disclosed herein can be delivered into hematopoietic stem cells, for example, by the methods as described, for example, in U.S. Pat. No. 5,928,638.

The ceDNA vectors in accordance with the present invention can be added to liposomes for delivery to a cell or target organ in a subject. Liposomes are vesicles that possess at least one lipid bilayer. Liposomes are typical used as carriers for drug/therapeutic delivery in the context of pharmaceutical development. They work by fusing with a cellular membrane and repositioning its lipid structure to deliver a drug or active pharmaceutical ingredient (API). Liposome compositions for such delivery are composed of phospholipids, especially compounds having a phosphatidylcholine group, however these compositions may also include other lipids. Exemplary liposomes and liposome formulations, including but not limited to polyethylene glycol (PEG)-functional group containing compounds are disclosed in International Application PCT/US2018/050042, filed on Sep. 7, 2018 and in International application PCT/US2018/064242, filed on Dec. 6, 2018, e.g., see the section entitled “Pharmaceutical Formulations”.]

Various delivery methods known in the art or modification thereof can be used to deliver ceDNA vectors in vitro or in vivo. For example, in some embodiments, ceDNA vectors are delivered by making transient penetration in cell membrane by mechanical, electrical, ultrasonic, hydrodynamic, or laser-based energy so that DNA entrance into the targeted cells is facilitated. For example, a ceDNA vector for insertion of a transgene at a GSH locus can be delivered by transiently disrupting cell membrane by squeezing the cell through a size-restricted channel or by other means known in the art. In some cases, a ceDNA vector alone is directly injected as naked DNA into skin, thymus, cardiac muscle, skeletal muscle, or liver cells. In some cases, a ceDNA vector is delivered by gene gun. Gold or tungsten spherical particles (1-3 μm diameter) coated with capsid-free AAV vectors can be accelerated to high speed by pressurized gas to penetrate into target tissue cells.

Compositions comprising a ceDNA vector for insertion of a transgene at a GSH locus and a pharmaceutically acceptable carrier are specifically contemplated herein. In some embodiments, the ceDNA vector for insertion of a transgene at a GSH locus is formulated with a lipid delivery system, for example, liposomes as described herein. In some embodiments, such compositions are administered by any route desired by a skilled practitioner. The compositions may be administered to a subject by different routes including orally, parenterally, sublingually, transdermally, rectally, transmucosally, topically, via inhalation, via buccal administration, intrapleurally, intravenous, intra-arterial, intraperitoneal, subcutaneous, intramuscular, intranasal intrathecal, and intraarticular or combinations thereof. For veterinary use, the composition may be administered as a suitably acceptable formulation in accordance with normal veterinary practice. The veterinarian may readily determine the dosing regimen and route of administration that is most appropriate for a particular animal. The compositions may be administered by traditional syringes, needleless injection devices, “microprojectile bombardment gene guns”, or other physical methods such as electroporation (“EP”), hydrodynamic methods, or ultrasound.

In some cases, a ceDNA vector for insertion of a transgene at a GSH locus is delivered by hydrodynamic injection, which is a simple and highly efficient method for direct intracellular delivery of any water-soluble compounds and particles into internal organs and skeletal muscle in an entire limb.

In some cases, ceDNA vectors are delivered by ultrasound by making nanoscopic pores in membrane to facilitate intracellular delivery of DNA particles into cells of internal organs or tumors, so the size and concentration of plasmid DNA have great role in efficiency of the system. In some cases, ceDNA vectors are delivered by magnetofection by using magnetic fields to concentrate particles containing nucleic acid into the target cells.

In some cases, chemical delivery systems can be used, for example, by using nanomeric complexes, which include compaction of negatively charged nucleic acid by polycationic nanomeric particles, belonging to cationic liposome/micelle or cationic polymers. Cationic lipids used for the delivery method includes, but not limited to monovalent cationic lipids, polyvalent cationic lipids, guanidine containing compounds, cholesterol derivative compounds, cationic polymers, (e.g., poly(ethylenimine), poly-L-lysine, protamine, other cationic polymers), and lipid-polymer hybrid.

### A. Exosomes:

In some embodiments, a ceDNA vector for insertion of a transgene at a GSH locus as disclosed herein is delivered by being packaged in an exosome. Exosomes are small membrane vesicles of endocytic origin that are released into the extracellular environment following fusion of multivesicular bodies with the plasma membrane. Their surface consists of a lipid bilayer from the donor cell's cell membrane, they contain cytosol from the cell that produced the exosome, and exhibit membrane proteins from the parental cell on the surface. Exosomes are produced by various cell types including epithelial cells, B and T lymphocytes, mast cells (MC) as well as dendritic cells (DC). Some embodiments, exosomes with a diameter between 10 nm and 1 μm, between 20 nm and 500 nm, between 30 nm and 250 nm, between 50 nm and 100 nm are envisioned for use. Exosomes can be isolated for a delivery to target cells using either their donor cells or by introducing specific nucleic acids into them. Various approaches known in the art can be used to produce exosomes containing capsid-free AAV vectors of the present invention.

### B. Microparticle/Nanoparticles:

In some embodiments, a ceDNA vector for insertion of a transgene at a GSH locus as disclosed herein is delivered by a lipid nanoparticle. Generally, lipid nanoparticles comprise an ionizable amino lipid (e.g., heptatriaconta-6,9,28,31-tetraen-19-yl 4-(dimethylamino)butanoate, DLin-MC3-DMA, a phosphatidylcholine (1,2-distearoyl-sn-glycero-3-phosphocholine, DSPC), cholesterol and a coat lipid (polyethylene glycol-dimyristolglycerol, PEG-DMG), for example as disclosed by Tam et al. (2013). Advances in Lipid Nanoparticles for siRNA delivery. Pharmaceuticals 5(3): 498-507.

In some embodiments, a lipid nanoparticle has a mean diameter between about 10 and about 1000 nm. In some embodiments, a lipid nanoparticle has a diameter that is less than 300 nm. In some embodiments, a lipid nanoparticle has a diameter between about 10 and about 300 nm. In some embodiments, a lipid nanoparticle has a diameter that is less than 200 nm. In some embodiments, a lipid nanoparticle has a diameter between about 25 and about 200 nm. In some embodiments, a lipid nanoparticle preparation (e.g., composition comprising a plurality of lipid nanoparticles) has a size distribution in which the mean size (e.g., diameter) is about 70 nm to about 200 nm, and more typically the mean size is about 100 nm or less.

Various lipid nanoparticles known in the art can be used to deliver ceDNA vector for insertion of a transgene at a GSH locus disclosed herein. For example, various delivery methods using lipid nanoparticles are described in U.S. Pat. Nos. 9,404,127, 9,006,417 and 9,518,272.

In some embodiments, a ceDNA vector for insertion of a transgene at a GSH locus disclosed herein is delivered by a gold nanoparticle. Generally, a nucleic acid can be covalently bound to a gold nanoparticle or non-covalently bound to a gold nanoparticle (e.g., bound by a charge-charge interaction), for example as described by Ding et al. (2014). Gold Nanoparticles for Nucleic Acid Delivery. Mol. Ther. 22(6); 1075-1083. In some embodiments, gold nanoparticle-nucleic acid conjugates are produced using methods described, for example, in U.S. Pat. No. 6,812,334.

### C. Conjugates

In some embodiments, a ceDNA vector for insertion of a transgene at a GSH locus as disclosed herein is conjugated (e.g., covalently bound to an agent that increases cellular uptake. An “agent that increases cellular uptake” is a molecule that facilitates transport of a nucleic acid across a lipid membrane. For example, a nucleic acid can be conjugated to a lipophilic compound (e.g., cholesterol, tocopherol, etc.), a cell penetrating peptide (CPP) (e.g., penetratin, TAT, Syn1B, etc.), and polyamines (e.g., spermine). Further examples of agents that increase cellular uptake are disclosed, for example, in Winkler (2013). Oligonucleotide conjugates for therapeutic applications. Ther. Deliv. 4(7); 791-809.

In some embodiments, a ceDNA vector for insertion of a transgene at a GSH locus as disclosed herein is conjugated to a polymer (e.g., a polymeric molecule) or a folate molecule (e.g., folic acid molecule). Generally, delivery of nucleic acids conjugated to polymers is known in the art, for example as described in WO2000/34343 and WO2008/022309. In some embodiments, a ceDNA vector for insertion of a transgene at a GSH locus as disclosed herein is conjugated to a poly(amide) polymer, for example as described by U.S. Pat. No. 8,987,377. In some embodiments, a nucleic acid described by the disclosure is conjugated to a folic acid molecule as described in U.S. Pat. No. 8,507,455.

In some embodiments, a ceDNA vector for insertion of a transgene at a GSH locus as disclosed herein is conjugated to a carbohydrate, for example as described in U.S. Pat. No. 8,450,467.

### D. Nanocapsule

Alternatively, nanocapsule formulations of a ceDNA vector for insertion of a transgene at a GSH locus as disclosed herein can be used. Nanocapsules can generally entrap substances in a stable and reproducible way. To avoid side effects due to intracellular polymeric overloading, such ultrafine particles (sized around 0.1 μm) should be designed using polymers able to be degraded in vivo. Biodegradable polyalkyl-cyanoacrylate nanoparticles that meet these requirements are contemplated for use.

### E. Liposomes

The ceDNA vectors in accordance with the present invention can be added to liposomes for delivery to a cell or target organ in a subject. Liposomes are vesicles that possess at least one lipid bilayer. Liposomes are typical used as carriers for drug/therapeutic delivery in the context of pharmaceutical development. They work by fusing with a cellular membrane and repositioning its lipid structure to deliver a drug or active pharmaceutical ingredient (API). Liposome compositions for such delivery are composed of phospholipids, especially compounds having a phosphatidylcholine group, however these compositions may also include other lipids.

The formation and use of liposomes is generally known to those of skill in the art. Liposomes have been developed with improved serum stability and circulation half-times (U.S. Pat. No. 5,741,516). Further, various methods of liposome and liposome like preparations as potential drug carriers have been described (U.S. Pat. Nos. 5,567,434; 5,552,157; 5,565,213; 5,738,868 and 5,795,587).

### F. Exemplary Liposome and Lipid Nanoparticle (LNP) Compositions

The ceDNA vectors in accordance with the present invention can be added to liposomes for delivery to a cell, e.g., a cell in need of expression of the transgene. Liposomes are vesicles that possess at least one lipid bilayer. Liposomes are typical used as carriers for drug/therapeutic delivery in the context of pharmaceutical development. They work by fusing with a cellular membrane and repositioning its lipid structure to deliver a drug or active pharmaceutical ingredient (API). Liposome compositions for such delivery are composed of phospholipids, especially compounds having a phosphatidylcholine group, however these compositions may also include other lipids.

Lipid nanoparticles (LNPs) comprising ceDNA are disclosed in International Application PCT/US2018/050042, filed on Sep. 7, 2018, and International Application PCT/US2018/064242, filed on Dec. 6, 2018 which are incorporated herein in their entirety and envisioned for use in the methods and compostions as disclosed herein.

In some aspects, a lipid nanoparticle comprising a ceDNA is an ionizable lipid.

Generally, the lipid particles are prepared at a total lipid to ceDNA (mass or weight) ratio of from about 10:1 to 30:1. In some embodiments, the lipid to ceDNA ratio (mass/mass ratio; w/w ratio) can be in the range of from about 1:1 to about 25:1, from about 10:1 to about 14:1, from about 3:1 to about 15:1, from about 4:1 to about 10:1, from about 5:1 to about 9:1, or about 6:1 to about 9:1. The amounts of lipids and ceDNA can be adjusted to provide a desired N/P ratio, for example, N/P ratio of 3, 4, 5, 6, 7, 8, 9, 10 or higher. Generally, the lipid particle formulation's overall lipid content can range from about 5 mg/ml to about 30 mg/mL. Ionizable lipids are also referred to as cationic lipids herein. Exemplary ionizable lipids are described in International PCT patent publications WO2015/095340, WO2015/199952, WO2018/011633, WO2017/049245, WO2015/061467, WO2012/040184, WO2012/000104, WO2015/074085, WO2016/081029, WO2017/004143, WO2017/075531, WO2017/117528, WO2011/022460, WO2013/148541, WO2013/116126, WO2011/153120, WO2012/044638, WO2012/054365, WO2011/090965, WO2013/016058, WO2012/162210, WO2008/042973, WO2010/129709, WO2010/144740, WO2012/099755, WO2013/049328, WO2013/086322, WO2013/086373, WO2011/071860, WO2009/132131, WO2010/048536, WO2010/088537, WO2010/054401, WO2010/054406, WO2010/054405, WO2010/054384, WO2012/016184, WO2009/086558, WO2010/042877, WO2011/000106, WO2011/000107, WO2005/120152, WO2011/141705, WO2013/126803, WO2006/007712, WO2011/038160, WO2005/121348, WO2011/066651, WO2009/127060, WO2011/141704, WO2006/069782, WO2012/031043, WO2013/006825, WO2013/033563, WO2013/089151, WO2017/099823, WO2015/095346, and WO2013/086354, and US patent publications US2016/0311759, US2015/0376115, US2016/0151284, US2017/0210697, US2015/0140070, US2013/0178541, US2013/0303587, US2015/0141678, US2015/0239926, US2016/0376224, U52017/0119904, US2012/0149894, US2015/0057373, US2013/0090372, US2013/0274523, US2013/0274504, US2013/0274504, US2009/0023673, US2012/0128760, US2010/0324120, US2014/0200257, US2015/0203446, US2018/0005363, US2014/0308304, US2013/0338210, US2012/0101148, US2012/0027796, US2012/0058144, US2013/0323269, US2011/0117125, US2011/0256175, US2012/0202871, U52011/0076335, US2006/0083780, US2013/0123338, US2015/0064242, US2006/0051405, US2013/0065939, US2006/0008910, US2003/0022649, US2010/0130588, US2013/0116307, US2010/0062967, US2013/0202684, US2014/0141070, US2014/0255472, US2014/0039032, US2018/0028664, US2016/0317458, and US2013/0195920, the contents of all of which are incorporated herein by reference in their entirety.

In some embodiments, the ionizable lipid is MC3 (6Z,9Z,28Z,31Z)-heptatriaconta-6,9,28,31-tetraen-19-yl-4-(dimethylamino) butanoate (DLin-MC3-DMA or MC3) having the following structure:

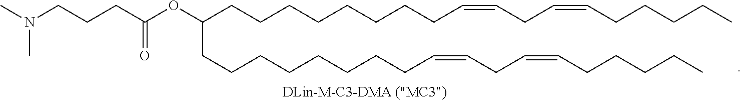 [SMILES: CCCCC/C=C\C/C=C\CCCCCCCCC(CCCCCCCC/C=C\C/C=C\CCCCC)OC(=O)CCCN(C)C]

VIII. Methods of Delivering ceDNA Vectors

In some embodiments, a ceDNA vector for insertion of a transgene at a GSH locus can be delivered to a target cell in vitro or in vivo by various suitable methods. ceDNA vectors alone can be applied or injected. CeDNA vectors can be delivered to a cell without the help of a transfection reagent or other physical means. Alternatively, ceDNA vectors can be delivered using any art-known transfection reagent or other art-known physical means that facilitates entry of DNA into a cell, e.g., liposomes, alcohols, polylysine-rich compounds, arginine-rich compounds, calcium phosphate, microvesicles, microinjection, electroporation and the like.

In contrast, transductions with capsid-free AAV vectors disclosed herein can efficiently target cell and tissue-types that are difficult to transduce with conventional AAV virions using various delivery reagent.

In another embodiment, a ceDNA vector for insertion of a transgene at a GSH locus is administered to the CNS (e.g., to the brain or to the eye). The ceDNA vector for insertion of a transgene at a GSH locus may be introduced into the spinal cord, brainstem (medulla oblongata, pons), midbrain (hypothalamus, thalamus, epithalamus, pituitary gland, substantia nigra, pineal gland), cerebellum, telencephalon (corpus striatum, cerebrum including the occipital, temporal, parietal and frontal lobes, cortex, basal ganglia, hippocampus and portaamygdala), limbic system, neocortex, corpus striatum, cerebrum, and inferior colliculus. The ceDNA vector may also be administered to different regions of the eye such as the retina, cornea and/or optic nerve. The ceDNA vector may be delivered into the cerebrospinal fluid (e.g., by lumbar puncture). The ceDNA vector may further be administered intravascularly to the CNS in situations in which the blood-brain barrier has been perturbed (e.g., brain tumor or cerebral infarct).

In some embodiments, the ceDNA vector for insertion of a transgene at a GSH locus can be administered to the desired region(s) of the CNS by any route known in the art, including but not limited to, intrathecal, intra-ocular, intracerebral, intraventricular, intravenous (e.g., in the presence of a sugar such as mannitol), intranasal, intra-aural, intra-ocular (e.g., intra-vitreous, sub-retinal, anterior chamber) and peri-ocular (e.g., sub-Tenon's region) delivery as well as intramuscular delivery with retrograde delivery to motor neurons.

In some embodiments, the ceDNA vector for insertion of a transgene at a GSH locus is administered in a liquid formulation by direct injection (e.g., stereotactic injection) to the desired region or compartment in the CNS. In other embodiments, the ceDNA vector can be provided by topical application to the desired region or by intra-nasal administration of an aerosol formulation. Administration to the eye may be by topical application of liquid droplets. As a further alternative, the ceDNA vector can be administered as a solid, slow-release formulation (see, e.g., U.S. Pat. No. 7,201,898). In yet additional embodiments, the ceDNA vector can used for retrograde transport to treat, ameliorate, and/or prevent diseases and disorders involving motor neurons (e.g., amyotrophic lateral sclerosis (ALS); spinal muscular atrophy (SMA), etc.). For example, the ceDNA vector can be delivered to muscle tissue from which it can migrate into neurons.

IX. Additional Uses of the ceDNA Vectors

The compositions and ceDNA vectors as described herein can be used to express a target gene or transgene for various purposes. In some embodiments, the resulting transgene encodes a protein or functional RNA that is intended to be used for research purposes, e.g., to create a somatic transgenic animal model harboring the transgene, e.g., to study the function of the transgene product. In another example, the transgene encodes a protein or functional RNA that is intended to be used to create an animal model of disease. In some embodiments, the resulting transgene encodes one or more peptides, polypeptides, or proteins, which are useful for the treatment, prevention, or amelioration of disease states or disorders in a mammalian subject. The resulting transgene can be transferred (e.g., expressed in) to a subject in a sufficient amount to treat a disease associated with reduced expression, lack of expression or dysfunction of the gene. In some embodiments the resulting transgene can be expressed in a subject in a sufficient amount to treat a disease associated with increased expression, activity of the gene product, or inappropriate upregulation of a gene that the resulting transgene suppresses or otherwise causes the expression of which to be reduced. In yet other embodiments, the resulting transgene replaces or supplements a defective copy of the native gene. It will be appreciated by one of ordinary skill in the art that the transgene may not be an open reading frame of a gene to be transcribed itself; instead it may be a promoter region or repressor region of a target gene, and the ceDNA vector may modify such region with the outcome of so modulating the expression of a gene of interest.

In some embodiments, the transgene encodes a protein or functional RNA that is intended to be used to create an animal model of disease. In some embodiments, the transgene encodes one or more peptides, polypeptides, or proteins, which are useful for the treatment or prevention of disease states in a mammalian subject. The transgene can be transferred (e.g., expressed in) to a patient in a sufficient amount to treat a disease associated with reduced expression, lack of expression or dysfunction of the gene.

### X. Methods of Use

A ceDNA vector for insertion of a transgene at a GSH locus as disclosed herein can also be used in a method for the delivery of a nucleotide sequence of interest (e.g., a transgene) to a target cell (e.g., a host cell). The method may in particular be a method for delivering a transgene to a cell of a subject in need thereof and treating a disease of interest. The invention allows for the in vivo expression of a transgene, e.g., a protein, antibody, nucleic acid such as miRNA etc. encoded in the ceDNA vector in a cell in a subject such that therapeutic effect of the expression of the transgene occurs. These results are seen with both in vivo and in vitro modes of ceDNA vector delivery.

In addition, the invention provides a method for the delivery of a transgene in a cell of a subject in need thereof, comprising multiple administrations of the ceDNA vector of the invention comprising said nucleic acid or transgene of interest to titrate the transgene expression to the desired level.

The ceDNA vector nucleic acid(s) are administered in sufficient amounts to transfect the cells of a desired tissue and to provide sufficient levels of gene transfer and expression without undue adverse effects. Conventional and pharmaceutically acceptable routes of administration include, but are not limited to, intravenous (e.g., in a liposome formulation), direct delivery to the selected organ (e.g., intraportal delivery to the liver), intramuscular, and other parental routes of administration. Routes of administration may be combined, if desired.

Closed-ended DNA vector (e.g. ceDNA vector) delivery is not limited to delivery gene replacements. For example, conventionally produced (e.g., using a cell-based production method or synthetically produced closed-ended DNA vectors) (e.g., ceDNA vectors) as described herein may be used with other delivery systems provided to provide a portion of the gene therapy. One non-limiting example of a system that may be combined with the synthetically produced ceDNA vectors in accordance with the present disclosure includes systems which separately deliver one or more co-factors or immune suppressors for effective gene expression of the transgene.

The invention also provides for a method of treating a disease in a subject comprising introducing into a target cell in need thereof (in particular a muscle cell or tissue) of the subject a therapeutically effective amount of a ceDNA vector, optionally with a pharmaceutically acceptable carrier. While the ceDNA vector for insertion of a transgene at a GSH locus can be introduced in the presence of a carrier, such a carrier is not required. The ceDNA vector selected comprises a nucleotide sequence of interest useful for treating the disease. In particular, the ceDNA vector may comprise a desired exogenous DNA sequence operably linked to control elements capable of directing transcription of the desired polypeptide, protein, or oligonucleotide encoded by the exogenous DNA sequence when introduced into the subject. The ceDNA vector can be administered via any suitable route as provided above, and elsewhere herein.

The compositions and vectors provided herein can be used to deliver a transgene for various purposes. In some embodiments, the transgene encodes a protein or functional RNA that is intended to be used for research purposes, e.g., to create a somatic transgenic animal model harboring the transgene, e.g., to study the function of the transgene product. In another example, the transgene encodes a protein or functional RNA that is intended to be used to create an animal model of disease. In some embodiments, the transgene encodes one or more peptides, polypeptides, or proteins, which are useful for the treatment or prevention of disease states in a mammalian subject. The transgene can be transferred (e.g., expressed in) to a patient in a sufficient amount to treat a disease associated with reduced expression, lack of expression or dysfunction of the gene.

In principle, the expression cassette can include a nucleic acid or any transgene that encodes a protein or polypeptide that is either reduced or absent due to a mutation or which conveys a therapeutic benefit when overexpressed is considered to be within the scope of the invention. Preferably, noninserted bacterial DNA is not present and preferably no bacterial DNA is present in the ceDNA compositions provided herein.

A ceDNA vector for insertion of a transgene at a GSH locus is not limited to one species of ceDNA vector. As such, in another aspect, multiple ceDNA vectors comprising different transgenes or the same transgene but operatively linked to different promoters or cis-regulatory elements can be delivered simultaneously or sequentially to the target cell, tissue, organ, or subject. Therefore, this strategy can allow for the gene therapy or gene delivery of multiple genes simultaneously. It is also possible to separate different portions of the transgene into separate ceDNA vectors (e.g., different domains and/or co-factors required for functionality of the transgene) which can be administered simultaneously or at different times, and can be separately regulatable, thereby adding an additional level of control of expression of the transgene. Delivery can also be performed multiple times and, importantly for gene therapy in the clinical setting, in subsequent increasing or decreasing doses, given the lack of an anti-capsid host immune response due to the absence of a viral capsid. It is anticipated that no anti-capsid response will occur as there is no capsid.

The invention also provides for a method of treating a disease in a subject comprising introducing into a target cell in need thereof (in particular a muscle cell or tissue) of the subject a therapeutically effective amount of a ceDNA vector as disclosed herein, optionally with a pharmaceutically acceptable carrier. While the ceDNA vector can be introduced in the presence of a carrier, such a carrier is not required. The ceDNA vector implemented comprises a nucleotide sequence of interest useful for treating the disease. In particular, the ceDNA vector may comprise a desired exogenous DNA sequence operably linked to control elements capable of directing transcription of the desired polypeptide, protein, or oligonucleotide encoded by the exogenous DNA sequence when introduced into the subject. The ceDNA vector for insertion of a transgene at a GSH locus can be administered via any suitable route as provided above, and elsewhere herein.

### XI. Methods of Treatment

The technology described herein also demonstrates methods for making, as well as methods of using the disclosed ceDNA vectors in a variety of ways, including, for example, ex situ, in vitro and in vivo applications, methodologies, diagnostic procedures, and/or gene therapy regimens.

Provided herein is a method of treating a disease or disorder in a subject comprising introducing into a target cell in need thereof (for example, a muscle cell or tissue, or other affected cell type) of the subject a therapeutically effective amount of a ceDNA vector, optionally with a pharmaceutically acceptable carrier. While the ceDNA vector can be introduced in the presence of a carrier, such a carrier is not required. The ceDNA vector implemented comprises a nucleotide sequence of interest useful for treating the disease. In particular, the ceDNA vector may comprise a desired exogenous DNA sequence operably linked to control elements capable of directing transcription of the desired polypeptide, protein, or oligonucleotide encoded by the exogenous DNA sequence when introduced into the subject. The ceDNA vector for insertion of a transgene at a GSH locus can be administered via any suitable route as provided above, and elsewhere herein.

Disclosed herein are ceDNA vector compositions and formulations that include one or more of the ceDNA vectors of the present invention together with one or more pharmaceutically-acceptable buffers, diluents, or excipients. Such compositions may be included in one or more diagnostic or therapeutic kits, for diagnosing, preventing, treating or ameliorating one or more symptoms of a disease, injury, disorder, trauma or dysfunction. In one aspect the disease, injury, disorder, trauma or dysfunction is a human disease, injury, disorder, trauma or dysfunction.

Another aspect of the technology described herein provides a method for providing a subject in need thereof with a diagnostically- or therapeutically-effective amount of a ceDNA vector, the method comprising providing to a cell, tissue or organ of a subject in need thereof, an amount of the ceDNA vector as disclosed herein; and for a time effective to enable expression of the transgene from the ceDNA vector thereby providing the subject with a diagnostically- or a therapeutically-effective amount of the protein, peptide, nucleic acid expressed by the ceDNA vector. In a further aspect, the subject is human.

Another aspect of the technology described herein provides a method for diagnosing, preventing, treating, or ameliorating at least one or more symptoms of a disease, a disorder, a dysfunction, an injury, an abnormal condition, or trauma in a subject. In an overall and general sense, the method includes at least the step of administering to a subject in need thereof one or more of the disclosed ceDNA vectors, in an amount and for a time sufficient to diagnose, prevent, treat or ameliorate the one or more symptoms of the disease, disorder, dysfunction, injury, abnormal condition, or trauma in the subject. In a further aspect, the subject is human.

Another aspect is use of the ceDNA vector for insertion of a transgene at a GSH locus as a tool for treating or reducing one or more symptoms of a disease or disease states. There are a number of inherited diseases in which defective genes are known, and typically fall into two classes: deficiency states, usually of enzymes, which are generally inherited in a recessive manner, and unbalanced states, which may involve regulatory or structural proteins, and which are typically but not always inherited in a dominant manner. For deficiency state diseases, ceDNA vectors can be used to deliver transgenes to bring a normal gene into affected tissues for replacement therapy, as well, in some embodiments, to create animal models for the disease using antisense mutations. For unbalanced disease states, ceDNA vectors can be used to create a disease state in a model system, which could then be used in efforts to counteract the disease state. Thus the ceDNA vectors and methods disclosed herein permit the treatment of genetic diseases. As used herein, a disease state is treated by partially or wholly remedying the deficiency or imbalance that causes the disease or makes it more severe. A. Host cells:

In some embodiments, the ceDNA vector for insertion of a transgene at a GSH locus delivers the transgene into a subject host cell. In some embodiments, the subject host cell is a human host cell, including, for example blood cells, stem cells, hematopoietic cells, CD34⁺ cells, liver cells, cancer cells, vascular cells, muscle cells, pancreatic cells, neural cells, ocular or retinal cells, epithelial or endothelial cells, dendritic cells, fibroblasts, or any other cell of mammalian origin, including, without limitation, hepatic (i.e., liver) cells, lung cells, cardiac cells, pancreatic cells, intestinal cells, diaphragmatic cells, renal (i.e., kidney) cells, neural cells, blood cells, bone marrow cells, or any one or more selected tissues of a subject for which gene therapy is contemplated. In one aspect, the subject host cell is a human host cell.

The present disclosure also relates to recombinant host cells as mentioned above, including ceDNA vectors as described herein. Thus, one can use multiple host cells depending on the purpose as is obvious to the skilled artisan. A construct or ceDNA vector for insertion of a transgene at a GSH locus including donor sequence is introduced into a host cell so that the donor sequence is maintained as a chromosomal integrant as described earlier. The term host cell encompasses any progeny of a parent cell that is not identical to the parent cell due to mutations that occur during replication. The choice of a host cell will to a large extent depend upon the donor sequence and its source. The host cell may also be a eukaryote, such as a mammalian, insect, plant, or fungal cell. In one embodiment, the host cell is a human cell (e.g., a primary cell, a stem cell, or an immortalized cell line). In some embodiments, the host cell can be administered the ceDNA vector for insertion of a transgene at a GSH locus ex vivo and then delivered to the subject after the gene therapy event. A host cell can be any cell type, e.g., a somatic cell or a stem cell, an induced pluripotent stem cell, or a blood cell, e.g., T-cell or B-cell, or bone marrow cell. In certain embodiments, the host cell is an allogenic cell. For example, T-cell genome engineering is useful for cancer immunotherapies, disease modulation such as HIV therapy (e.g., receptor knock out, such as CXCR4 and CCR5) and immunodeficiency therapies. MHC receptors on B-cells can be targeted for immunotherapy. In some embodiments, gene modified host cells, e.g., bone marrow stem cells, e.g., CD34⁺ cells, or induced pluripotent stem cells can be transplanted back into a patient for expression of a therapeutic protein.

B. Exemplary Transgenes and Diseases to be Treated with a ceDNA Vector for Insertion of a Trangsnege at a GSH

In some embodiments, a ceDNA vector composition as described herein for integration of a nucleic acid of interest into a GSH locus comprises, between the restriction cloning sites, a nucleic acid of interest. In some embodiments, the nucleic acid of interest is gene editing nucleic acid sequence as disclosed herein, and in some embodiments, the nucleic acid of interest can be for example, a heterologous gene, a nucleic acid encoding a therapeutic protein, antibody, peptide, or an antisense oligonucleic acid, or the like.

In some embodiments, the nucleic acid of interest is a RNA, e.g., RNAi, antisense nucleic acid, miRNA and variants thereof. In some embodiments, a nucleic acid of interest may comprise any sequence of interest and can also be referred to herein as an “exogenous sequence”. Exemplary nucleic acid of interests include, but are not limited to any polypeptide coding sequence (e.g., cDNAs), promoter sequences, enhancer sequences, epitope tags, marker genes, cleavage enzyme recognition sites, epitope tags and various types of expression constructs. Marker genes include, but are not limited to, sequences encoding proteins that mediate antibiotic resistance (e.g., ampicillin resistance, neomycin resistance, G418 resistance, puromycin resistance), sequences encoding colored or fluorescent or luminescent proteins (e.g., green fluorescent protein, enhanced green fluorescent protein, red fluorescent protein, luciferase), and proteins which mediate cellular metabolism resulting in enhanced cell growth rates and/or gene amplification (e.g., dihydrofolate reductase). Epitope tags can be fused to a protein of interest to facilitate detection, and include, for example, one or more copies of FLAG, His, myc, Tap, HA or any detectable amino acid sequence.

In some embodiments, a nucleic acid of interest can comprise one or more sequences which do not encode polypeptides but rather any type of noncoding sequence, as well as one or more control elements (e.g., promoters). In addition, a nucleic acid of interest can produce one or more RNA molecules (e.g., small hairpin RNAs (shRNAs), inhibitory RNAs (RNAis), microRNAs (miRNAs), etc.).

In some embodiments, the nucleic acid of interest encodes a receptor, toxin, a hormone, an enzyme, or a cell surface protein or a therapeutic protein, peptide or antibody or fragment thereof. In some embodiments, a nucleic acid of interest for use in the ceDNA vector compositions as disclosed herein encodes any polypeptide of which expression in the cell is desired, including, but not limited to antibodies, antigens, enzymes, receptors (cell surface or nuclear), hormones, lymphokines, cytokines, reporter polypeptides, growth factors, and functional fragments of any of the above. The coding sequences may be, for example, cDNAs.

In certain embodiments, a nucleic acid of interest for use in the ceDNA vector as disclosed herein comprises a nucleic acid sequence that encodes a marker gene (described herein), allowing selection of cells that have undergone targeted integration, and a linked sequence encoding an additional functionality. Non-limiting examples of marker genes include GFP, drug selection marker(s) and the like.

Furthermore, although not required for expression, a nucleic acid of interest may also comprise a transcriptional or translational regulatory sequences, for example, promoters, enhancers, insulators, internal ribosome entry sites, sequences encoding 2A peptides and/or polyadenylation signals.

In some aspects, a nucleic acid of interest as defined herein encodes a nucleic acid for use in methods of preventing or treating one or more genetic deficiencies or dysfunctions in a mammal, such as for example, a polypeptide deficiency or polypeptide excess in a mammal, and particularly for treating or reducing the severity or extent of deficiency in a human manifesting one or more of the disorders linked to a deficiency in such polypeptides in cells and tissues. The method involves administration of the nucleic acid of interest (e.g., a nucleic acid as described by the disclosure) that encodes one or more therapeutic peptides, polypeptides, siRNAs, microRNAs, antisense nucleotides, etc. in a pharmaceutically-acceptable carrier to the subject in an amount and for a period of time sufficient to treat the deficiency or disorder in the subject suffering from such a disorder.

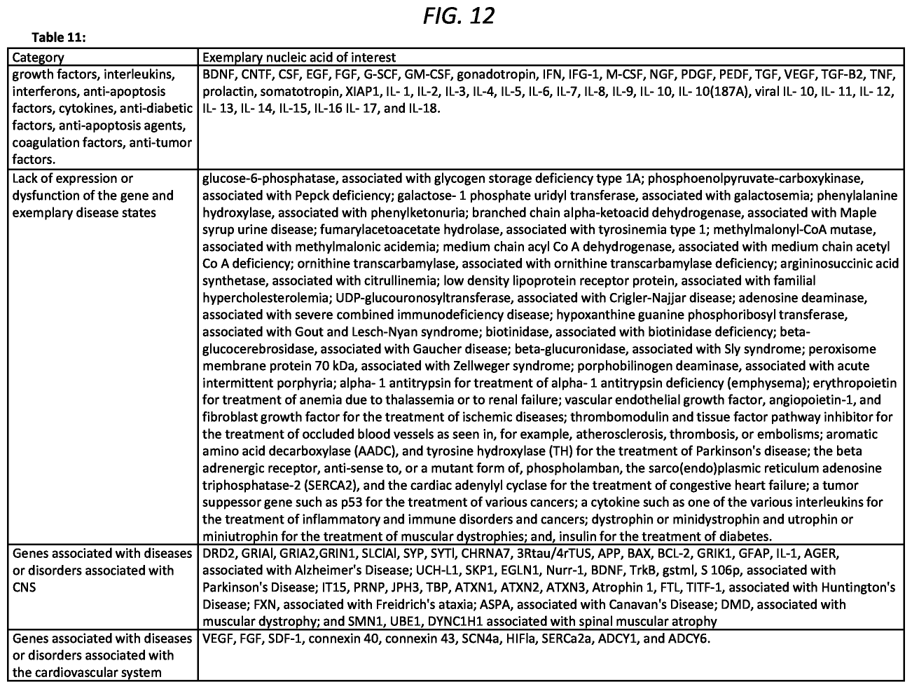

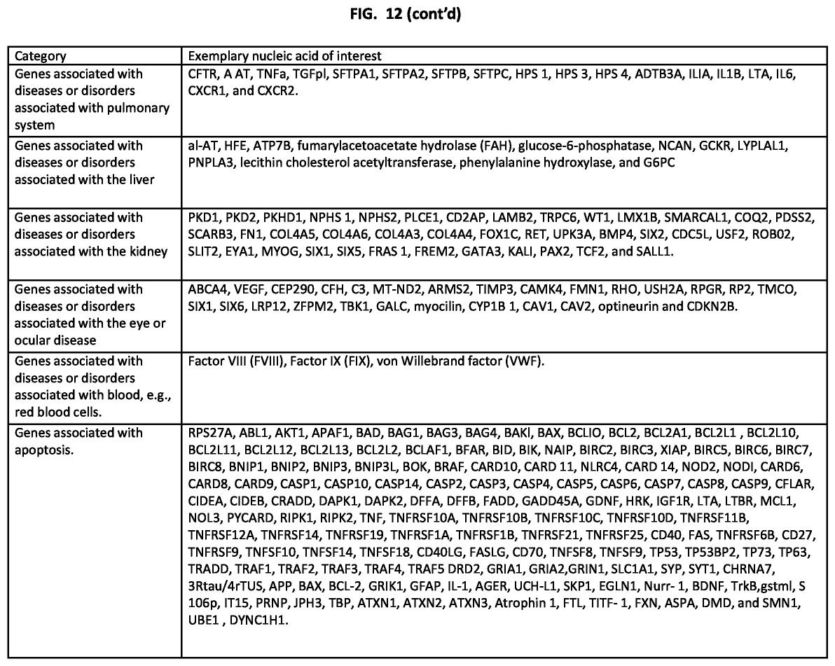

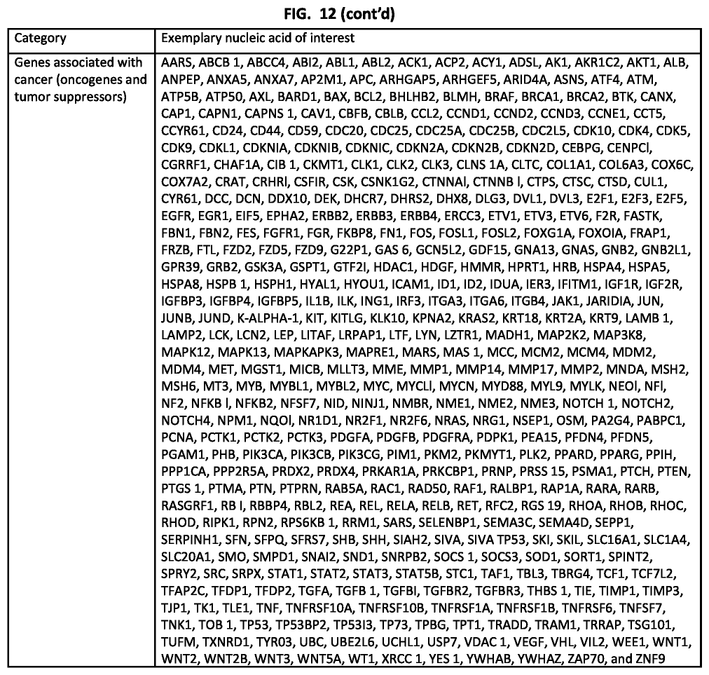

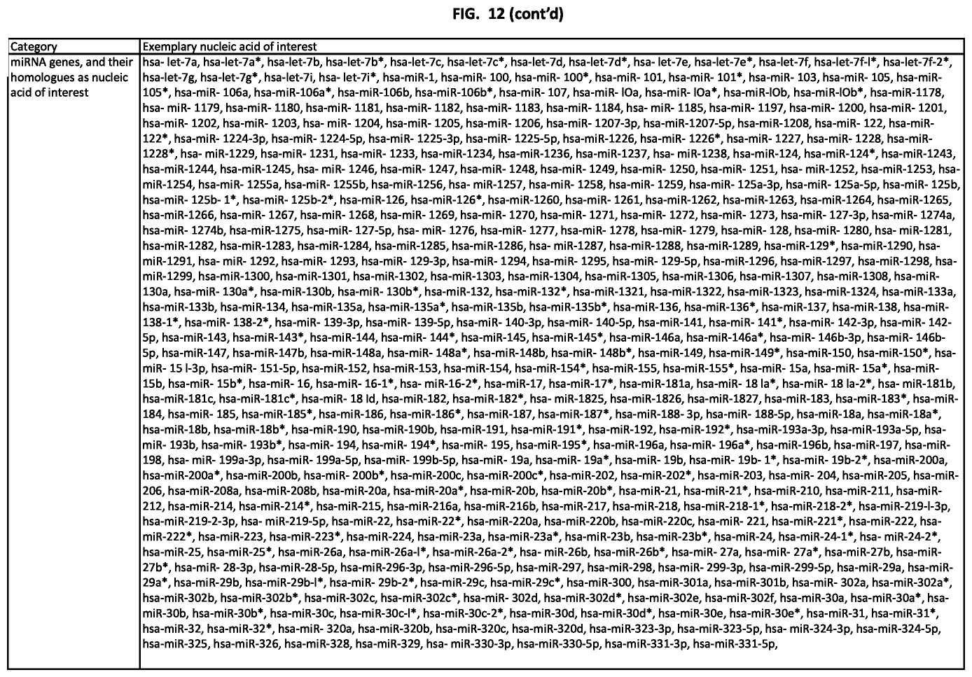

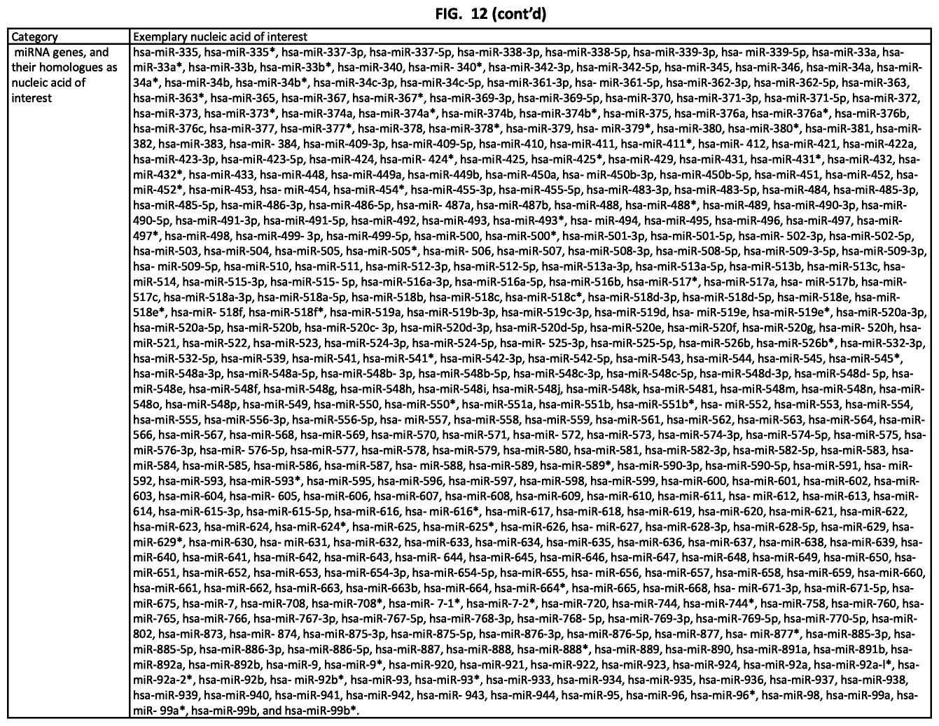

Thus in some embodiments, nucleic acids of interest for use in the ceDNA vector as disclosed herein can encode one or more peptides, polypeptides, or proteins, which are useful for the treatment or prevention of disease states in a mammalian subject. Exemplary nucleic acids of interest for use in the compositions and methods as disclosed herein are disclosed in the Table 11 in FIG. 12 herein. These include one or more polypeptides selected from the group consisting of growth factors, interleukins, interferons, anti-apoptosis factors, cytokines, anti-diabetic factors, anti-apoptosis agents, coagulation factors, anti-tumor factors.

In some embodiments, nucleic acids of interest for use in ceDNA vector as disclosed herein may encode a gene, or part of a gene to be transferred (e.g., expressed in) in a subject to treat a disease associated with reduced expression, lack of expression or dysfunction of the gene. Exemplary genes and associated disease states are disclosed herein.

The ceDNA vectors are also useful for correcting a defective gene. As a non-limiting example, DMD gene of Duchene Muscular Dystrophy can be delivered using the ceDNA vectors as disclosed herein.

A ceDNA vector for insertion of a transgene at a GSH locus or a composition thereof can be used in the treatment of any hereditary disease. As a non-limiting example, the ceDNA vector or a composition thereof e.g. can be used in the treatment of transthyretin amyloidosis (ATTR), an orphan disease where the mutant protein misfolds and aggregates in nerves, the heart, the gastrointestinal system etc. It is contemplated herein that the disease can be treated by deletion of the mutant disease gene (mutTTR) using the ceDNA vector systems described herein. Such treatments of hereditary diseases can halt disease progression and may enable regression of an established disease or reduction of at least one symptom of the disease by at least 10%.

In another embodiment, a ceDNA vector for insertion of a transgene at a GSH locus can be used in the treatment of ornithine transcarbamylase deficiency (OTC deficiency), hyperammonaemia or other urea cycle disorders, which impair a neonate or infant's ability to detoxify ammonia. As with all diseases of inborn metabolism, it is contemplated herein that even a partial restoration of enzyme activity compared to wild-type controls (e.g., at least 20%, at least 30%, at least 40%, at least 50%, at least 60%, at least 70%, at least 80%, at least 90%, at least 95% or at least 99%) may be sufficient for reduction in at least one symptom OTC and/or an improvement in the quality of life for a subject having OTC deficiency. In one embodiment, a nucleic acid encoding OTC can be inserted behind the albumin endogenous promoter for in vivo protein replacement.

In another embodiment, a ceDNA vector for insertion of a transgene at a GSH locus can be used in the treatment of phenylketonuria (PKU) by delivering a nucleic acid sequence encoding a phenylalanine hydroxylase enzyme to reduce buildup of dietary phenylalanine, which can be toxic to PKU sufferers. As with all diseases of inborn metabolism, it is contemplated herein that even a partial restoration of enzyme activity compared to wild-type controls (e.g., at least 20%, at least 30%, at least 40%, at least 50%, at least 60%, at least 70%, at least 80%, at least 90%, at least 95% or at least 99%) may be sufficient for reduction in at least one symptom of PKU and/or an improvement in the quality of life for a subject having PKU. In one embodiment, a nucleic acid encoding phenylalanine hydroxylase can be inserted behind the albumin endogenous promoter for in vivo protein replacement.

In another embodiment, a ceDNA vector for insertion of a transgene at a GSH locus can be used in the treatment of glycogen storage disease (GSD) by delivering a nucleic acid sequence encoding an enzyme to correct aberrant glycogen synthesis or breakdown in subjects having GSD. Non-limiting examples of enzymes that can be delivered and expressed using the ceDNA vectors and methods as described herein include glycogen synthase, glucose-6-phosphatase, acid-alpha glucosidase, glycogen debranching enzyme, glycogen branching enzyme, muscle glycogen phosphorylase, liver glycogen phosphorylase, muscle phosphofructokinase, phosphorylase kinase, glucose transporter-2 (GLUT-2), aldolase A, beta-enolase, phosphoglucomutase-1 (PGM-1), and glycogenin-1. As with all diseases of inborn metabolism, it is contemplated herein that even a partial restoration of enzyme activity compared to wild-type controls (e.g., at least 20%, at least 30%, at least 40%, at least 50%, at least 60%, at least 70%, at least 80%, at least 90%, at least 95% or at least 99%) may be sufficient for reduction in at least one symptom of GSD and/or an improvement in the quality of life for a subject having GSD. In one embodiment, a nucleic acid encoding an enzyme to correct aberrant glycogen storage can be inserted behind the albumin endogenous promoter for in vivo protein replacement.

The ceDNA vectors described herein are also contemplated for use in the treatment of any of; of Leber congenital amaurosis (LCA), polyglutamine diseases, including polyQ repeats, and alpha-1 antitrypsin deficiency (A1AT). LCA is a rare congenital eye disease resulting in blindness, which can be caused by a mutation in any one of the following genes: GUCY2D, RPE65, SPATA7, AIPL1, LCA5, RPGRIP1, CRX, CRB1, NMNAT1, CEP290, IMPDH1, RD3, RDH12, LRAT, TULP1, KCNJ13, GDF6 and/or PRPH2. It is contemplated herein that the ceDNA vectors and compositions and methods as described herein can be adapted for delivery of one or more of the genes associated with LCA in order to correct an error in the gene(s) responsible for the symptoms of LCA. Polyglutamine diseases include, but are not limited to: dentatorubropallidoluysian atrophy, Huntington's disease, spinal and bulbar muscular atrophy, and spinocerebellar ataxia types 1, 2, 3 (also known as Machado-Joseph disease), 6, 7, and 17. A1AT deficiency is a genetic disorder that causes defective production of alpha-1 antitrypsin, leading to decreased activity of the enzyme in the blood and lungs, which in turn can lead to emphysema or chronic obstructive pulmonary disease in affected subjects. Treatment of a subject with an A1AT deficiency is specifically contemplated herein using the ceDNA vectors or compositions thereof as outlined herein. It is contemplated herein that a ceDNA vector for insertion of a transgene at a GSH locus as disclosed herein, comprising a nucleic acid encoding a desired protein for the treatment of LCA, polyglutamine diseases or A1AT deficiency can be administered to a subject in need of treatment.

In further embodiments, the compositions comprising a ceDNA vector for insertion of a transgene at a GSH locus as disclosed herein, can be used to deliver a viral sequence, a pathogen sequence, a chromosomal sequence, a translocation junction (e.g., a translocation associated with cancer), a non-coding RNA gene or RNA sequence, a disease associated gene, among others.

Any nucleic acid or target gene of interest may be delivered or expressed by a ceDNA vector for insertion of a transgene at a GSH locus as disclosed herein. Target nucleic acids and target genes include, but are not limited to nucleic acids encoding polypeptides, or non-coding nucleic acids (e.g., RNAi, miRs etc.) preferably therapeutic (e.g., for medical, diagnostic, or veterinary uses) or immunogenic (e.g., for vaccines) polypeptides. In certain embodiments, the target nucleic acids or target genes that are targeted by the ceDNA vectors as described herein encode one or more polypeptides, peptides, ribozymes, peptide nucleic acids, siRNAs, RNAis, antisense oligonucleotides, antisense polynucleotides, antibodies, antigen binding fragments, or any combination thereof.

In particular, a gene target or transgene for expression by the ceDNA vector for insertion of a transgene at a GSH locus as disclosed herein can encode, for example, but is not limited to, protein(s), polypeptide(s), peptide(s), enzyme(s), antibodies, antigen binding fragments, as well as variants, and/or active fragments thereof, for use in the treatment, prophylaxis, and/or amelioration of one or more symptoms of a disease, dysfunction, injury, and/or disorder. In one aspect, the disease, dysfunction, trauma, injury and/or disorder is a human disease, dysfunction, trauma, injury, and/or disorder.

The expression cassette can also encode polypeptides, sense or antisense oligonucleotides, or RNAs (coding or non-coding; e.g., siRNAs, shRNAs, micro-RNAs, and their antisense counterparts (e.g., antagoMiR)). Expression cassettes can include an exogenous sequence that encodes a reporter protein to be used for experimental or diagnostic purposes, such as β-lactamase, β-galactosidase (LacZ), alkaline phosphatase, thymidine kinase, green fluorescent protein (GFP), chloramphenicol acetyltransferase (CAT), luciferase, and others well known in the art.

Sequences provided in the expression cassette, expression construct of a ceDNA vector for insertion of a transgene at a GSH locus described herein can be codon optimized for the host cell. As used herein, the term “codon optimized” or “codon optimization” refers to the process of modifying a nucleic acid sequence for enhanced expression in the cells of the vertebrate of interest, e.g., mouse or human, by replacing at least one, more than one, or a significant number of codons of the native sequence (e.g., a prokaryotic sequence) with codons that are more frequently or most frequently used in the genes of that vertebrate. Various species exhibit particular bias for certain codons of a particular amino acid. Typically, codon optimization does not alter the amino acid sequence of the original translated protein. Optimized codons can be determined using e.g., Aptagen's Gene Forge® codon optimization and custom gene synthesis platform (Aptagen, Inc., 2190 Fox Mill Rd. Suite 300, Herndon, Va. 20171) or another publicly available database.

Many organisms display a bias for use of particular codons to code for insertion of a particular amino acid in a growing peptide chain. Codon preference or codon bias, differences in codon usage between organisms, is afforded by degeneracy of the genetic code, and is well documented among many organisms. Codon bias often correlates with the efficiency of translation of messenger RNA (mRNA), which is in turn believed to be dependent on, inter alia, the properties of the codons being translated and the availability of particular transfer RNA (tRNA) molecules. The predominance of selected tRNAs in a cell is generally a reflection of the codons used most frequently in peptide synthesis. Accordingly, genes can be tailored for optimal gene expression in a given organism based on codon optimization.

Given the large number of gene sequences available for a wide variety of animal, plant and microbial species, it is possible to calculate the relative frequencies of codon usage (Nakamura, Y., et al. “Codon usage tabulated from the international DNA sequence databases: status for the year 2000” Nucl. Acids Res. 28:292 (2000)).

As noted herein, a ceDNA vector for insertion of a transgene at a GSH locus as disclosed herein can encode a protein or peptide, or therapeutic nucleic acid sequence or therapeutic agent, including but not limited to one or more agonists, antagonists, anti-apoptosis factors, inhibitors, receptors, cytokines, cytotoxins, erythropoietic agents, glycoproteins, growth factors, growth factor receptors, hormones, hormone receptors, interferons, interleukins, interleukin receptors, nerve growth factors, neuroactive peptides, neuroactive peptide receptors, proteases, protease inhibitors, protein decarboxylases, protein kinases, protein kinase inhibitors, enzymes, receptor binding proteins, transport proteins or one or more inhibitors thereof, serotonin receptors, or one or more uptake inhibitors thereof, serpins, serpin receptors, tumor suppressors, diagnostic molecules, chemotherapeutic agents, cytotoxins, or any combination thereof.

The ceDNA vectors are also useful for ablating gene expression. For example, in one embodiment a ceDNA vector for insertion of a transgene at a GSH locus as described herein can be used to express an antisense nucleic acid or functional RNA to induce knockdown of a target gene. As a non-limiting example, expression of CXCR4 and CCR5, HIV receptors, have been successfully ablated in primary human T-cells, See Schumann et al. (2015), PNAS 112(33): 10437-10442, herein incorporated by reference in its entirety. Another gene for targeted inhibition is PD-1, where the ceDNA vector can express an inhibitory nucleic acid or RNAi or functional RNA to inhibit the expression of PD-1. PD-1 expresses an immune checkpoint cell surface receptor on chronically active T cells that happens in malignancy. See Schumann et al. supra.

In some embodiments, a ceDNA vectors is useful for correcting a defective gene by expressing a transgene that targets the diseased gene. Non-limiting examples of diseases or disorders amenable to treatment, by a ceDNA vector for insertion of a transgene at a GSH locus as disclosed herein, and the transgenes to be expressed are listed in Tables A-C of US patent publication 2014/0170753, which is herein incorporated by reference in its entirety.

In alternative embodiments, the ceDNA vectors are used for insertion of an expression cassette for expression of a therapeutic protein or reporter protein in a safe harbor gene, e.g., in an inactive intron. In certain embodiments, a promoter-less cassette is inserted into the safe harbor gene. In such embodiments, a promoter-less cassette can take advantage of the safe harbor gene regulatory elements (promoters, enhancers, and signaling peptides), a non-limiting example of insertion at the safe harbor locus is insertion into to the albumin locus that is described in Blood (2015) 126 (15): 1777-1784, which is incorporated herein by reference in its entirety. Insertion into Albumin has the benefit of enabling secretion of the transgene into the blood (See e.g., Example 22). In addition, a genomic safe harbor site can be determined using techniques known in the art and described in, for example, Papapetrou, ER & Schambach, A. Molecular Therapy 24(4):678-684 (2016) or Sadelain et al. Nature Reviews Cancer 12:51-58 (2012), the contents of each of which are incorporated herein by reference in their entirety. It is specifically contemplated herein that safe harbor sites in an adeno associated virus (AAV) genome (e.g., AAVS1 safe harbor site) can be used with the methods and compositions described herein (see e.g., Oceguera-Yanez et al. Methods 101:43-55 (2016) or Tiyaboonchai, A et al. Stem Cell Res 12(3):630-7 (2014), the contents of each of which are incorporated by reference in their entirety). For example, the AAVS1 genomic safe harbor site can be used with the ceDNA vectors and compositions as described herein for the purposes of hematopoietic specific transgene expression and gene silencing in embryonic stem cells (e.g., human embryonic stem cells) or induced pluripotent stem cells (iPS cells). In addition, it is contemplated herein that synthetic or commercially available homology-directed repair donor templates for insertion into an AASV1 safe harbor site on chromosome 19 can be used with the ceDNA vectors or compositions as described herein. For example, homology-directed repair templates, and guide RNA, can be purchased commercially, for example, from System Biosciences, Palo Alto, Calif., and cloned into a ceDNA vector.

In some embodiments, the ceDNA vectors are used for expressing a transgene, or knocking out or decreasing expression of a target gene in a T cell, e.g., to engineer the T cell for improved adoptive cell transfer and/or CAR-T therapies (see, e.g., Example 24). In some embodiments, the ceDNA vector for insertion of a transgene at a GSH locus as described herein can express transgenes that knock-out genes. Non-limiting examples of therapeutically relevant knock-outs of T cells are described in PNAS (2015) 112(33):10437-10442, which is incorporated herein by reference in its entirety.

### C. Additional Diseases for Gene Therapy:

In general, the ceDNA vector for insertion of a transgene at a GSH locus as disclosed herein can be used to deliver any transgene in accordance with the description above to treat, prevent, or ameliorate the symptoms associated with any disorder related to gene expression. Illustrative disease states include, but are not-limited to: cystic fibrosis (and other diseases of the lung), hemophilia A, hemophilia B, thalassemia, anemia and other blood disorders, AIDS, Alzheimer's disease, Parkinson's disease, Huntington's disease, amyotrophic lateral sclerosis, epilepsy, and other neurological disorders, cancer, diabetes mellitus, muscular dystrophies (e.g., Duchenne, Becker), Hurler's disease, adenosine deaminase deficiency, metabolic defects, retinal degenerative diseases (and other diseases of the eye), mitochondriopathies (e.g., Leber's hereditary optic neuropathy (LHON), Leigh syndrome, and subacute sclerosing encephalopathy), myopathies (e.g., facioscapulohumeral myopathy (FSHD) and cardiomyopathies), diseases of solid organs (e.g., brain, liver, kidney, heart), and the like. In some embodiments, the ceDNA vectors as disclosed herein can be advantageously used in the treatment of individuals with metabolic disorders (e.g., ornithine transcarbamylase deficiency).

In some embodiments, the ceDNA vector for insertion of a transgene at a GSH locus described herein can be used to treat, ameliorate, and/or prevent a disease or disorder caused by mutation in a gene or gene product. Exemplary diseases or disorders that can be treated with a ceDNA vectors include, but are not limited to, metabolic diseases or disorders (e.g., Fabry disease, Gaucher disease, phenylketonuria (PKU), glycogen storage disease); urea cycle diseases or disorders (e.g., ornithine transcarbamylase (OTC) deficiency); lysosomal storage diseases or disorders (e.g., metachromatic leukodystrophy (MLD), mucopolysaccharidosis Type II (MPSII; Hunter syndrome)); liver diseases or disorders (e.g., progressive familial intrahepatic cholestasis (PFIC); blood diseases or disorders (e.g., hemophilia (A and B), thalassemia, and anemia); cancers and tumors, and genetic diseases or disorders (e.g., cystic fibrosis).

In some embodiments, a ceDNA vector for insertion of a transgene into a GSH as disclosed herein comprises a nucleic acid sequence (cDNA or gDNA) that encodes a polypeptide that is lacking or non-functional in the subject having a genetic disease, including but not limited to any of the following genetic diseases selected from any of: achondroplasia, achromatopsia, acid maltase deficiency, adenosine deaminase deficiency (OMIM No. 102700), adrenoleukodystrophy, aicardi syndrome, alpha-1 antitrypsin deficiency, alpha-thalassemia, androgen insensitivity syndrome, apert syndrome, arrhythmogenic right ventricular, dysplasia, ataxia telangictasia, barth syndrome, beta-thalassemia, blue rubber bleb nevus syndrome, canavan disease, chronic granulomatous diseases (CGD), cri du chat syndrome, cystic fibrosis, dercum's disease, ectodermal dysplasia, fanconi anemia, fibrodysplasia ossificans progressive, fragile X syndrome, galactosemis, Gaucher's disease, generalized gangliosidoses (e.g., GM1), hemochromatosis, the hemoglobin C mutation in the 6th codon of beta-globin (HbC), hemophilia, Huntington's disease, Hurler Syndrome, hypophosphatasia, Klinefleter syndrome, Krabbes Disease, Langer-Giedion Syndrome, leukocyte adhesion deficiency (LAD, OMIM No. 116920), leukodystrophy, long QT syndrome, Marfan syndrome, Moebius syndrome, mucopolysaccharidosis (MPS), nail patella syndrome, nephrogenic diabetes insipdius, neurofibromatosis, Neimann-Pick disease, osteogenesis imperfecta, porphyria, Prader-Willi syndrome, progeria, Proteus syndrome, retinoblastoma, Rett syndrome, Rubinstein-Taybi syndrome, Sanfilippo syndrome, severe combined immunodeficiency (SCID), Shwachman syndrome, sickle cell disease (sickle cell anemia), Smith-Magenis syndrome, Stickler syndrome, Tay-Sachs disease, Thrombocytopenia Absent Radius (TAR) syndrome, Treacher Collins syndrome, trisomy, tuberous sclerosis, Turner's syndrome, urea cycle disorder, von Hippel-Landau disease, Waardenburg syndrome, Williams syndrome, Wilson's disease, Wiskott-Aldrich syndrome, X-linked lymphoproliferative syndrome (XLP, OMIM No. 308240). Additional exemplary diseases that can be treated by targeted integration include acquired immunodeficiencies, lysosomal storage diseases (e.g., Gaucher's disease, GM1, Fabry disease and Tay-Sachs disease), mucopolysaccahidosis (e.g. Hunter's disease, Hurler's disease), hemoglobinopathies (e.g., sickle cell diseases, HbC, α-thalassemia, β-thalassemia) and hemophilias.

As still a further aspect, a ceDNA vector for insertion of a transgene at a GSH locus as disclosed herein may be employed to deliver a heterologous nucleotide sequence in situations in which it is desirable to regulate the level of transgene expression (e.g., transgenes encoding hormones or growth factors, as described herein).

Accordingly, in some embodiments, the ceDNA vector for insertion of a transgene at a GSH locus sa described herein can be used to correct an abnormal level and/or function of a gene product (e.g., an absence of, or a defect in, a protein) that results in the disease or disorder. The ceDNA vector can produce a functional protein and/or modify levels of the protein to alleviate or reduce symptoms resulting from, or confer benefit to, a particular disease or disorder caused by the absence or a defect in the protein. For example, treatment of OTC deficiency can be achieved by producing functional OTC enzyme; treatment of hemophilia A and B can be achieved by modifying levels of Factor VIII, Factor IX, and Factor X; treatment of PKU can be achieved by modifying levels of phenylalanine hydroxylase enzyme; treatment of Fabry or Gaucher disease can be achieved by producing functional alpha galactosidase or beta glucocerebrosidase, respectively; treatment of MLD or MPSII can be achieved by producing functional arylsulfatase A or iduronate-2-sulfatase, respectively; treatment of cystic fibrosis can be achieved by producing functional cystic fibrosis transmembrane conductance regulator; treatment of glycogen storage disease can be achieved by restoring functional G6Pase enzyme function; and treatment of PFIC can be achieved by producing functional ATP8B1, ABCB11, ABCB4, or TJP2 genes.

In alternative embodiments, the ceDNA vectors as disclosed herein can be used to provide an antisense nucleic acid to a cell in vitro or in vivo. For example, where the transgene is a RNAi molecule, expression of the antisense nucleic acid or RNAi in the target cell diminishes expression of a particular protein by the cell. Accordingly, transgenes which are RNAi molecules or antisense nucleic acids may be administered to decrease expression of a particular protein in a subject in need thereof. Antisense nucleic acids may also be administered to cells in vitro to regulate cell physiology, e.g., to optimize cell or tissue culture systems.

In some embodiments, exemplary transgenes encoded by the ceDNA vector for insertion of a transgene at a GSH locus include, but are not limited to: X, lysosomal enzymes (e.g., hexosaminidase A, associated with Tay-Sachs disease, or iduronate sulfatase, associated, with Hunter Syndrome/MPS II), erythropoietin, angiostatin, endostatin, superoxide dismutase, globin, leptin, catalase, tyrosine hydroxylase, as well as cytokines (e.g., a interferon, β-interferon, interferon-γ, interleukin-2, interleukin-4, interleukin 12, granulocyte-macrophage colony stimulating factor, lymphotoxin, and the like), peptide growth factors and hormones (e.g., somatotropin, insulin, insulin-like growth factors 1 and 2, platelet derived growth factor (PDGF), epidermal growth factor (EGF), fibroblast growth factor (FGF), nerve growth factor (NGF), neurotrophic factor-3 and 4, brain-derived neurotrophic factor (BDNF), glial derived growth factor (GDNF), transforming growth factor-α and -β, and the like), receptors (e.g., tumor necrosis factor receptor).

In some exemplary embodiments, the transgene encodes a monoclonal antibody specific for one or more desired targets. Exemplary transgenes encompassed for use in a ceDNA vector for insertion of a transgene at a GSH locus as disclosed herein can be any antibody or fusion protein as disclosed in International Application PCT/US19/18016, filed on Feb. 14, 2019, which is incorporated herein in its entirety by reference.

In some exemplary embodiments, more than one transgene is encoded by the ceDNA vector. In some exemplary embodiments, the transgene encodes a fusion protein comprising two different polypeptides of interest. In some embodiments, the transgene encodes an antibody, including a full-length antibody or antibody fragment, as defined herein. In some embodiments, the antibody is an antigen-binding domain or an immunoglobulin variable domain sequence, as that is defined herein. Other illustrative transgene sequences encode suicide gene products (thymidine kinase, cytosine deaminase, diphtheria toxin, cytochrome P450, deoxycytidine kinase, and tumor necrosis factor), proteins conferring resistance to a drug used in cancer therapy, and tumor suppressor gene products.

In a representative embodiment, the transgene expressed by the ceDNA vector for insertion of a transgene at a GSH locus can be used for the treatment of muscular dystrophy in a subject in need thereof, the method comprising: administering a treatment-, amelioration- or prevention-effective amount of ceDNA vector described herein, wherein the ceDNA vector comprises a heterologous nucleic acid encoding dystrophin, a mini-dystrophin, a micro-dystrophin, myostatin propeptide, follistatin, activin type II soluble receptor, IGF-1, anti-inflammatory polypeptides such as the Ikappa B dominant mutant, sarcospan, utrophin, a micro-dystrophin, laminin-α2, α-sarcoglycan, β-sarcoglycan, γ-sarcoglycan, δ-sarcoglycan, IGF-1, an antibody or antibody fragment against myostatin or myostatin propeptide, and/or RNAi against myostatin. In particular embodiments, the ceDNA vector can be administered to skeletal, diaphragm and/or cardiac muscle as described elsewhere herein.

In some embodiments, the ceDNA vector for insertion of a transgene at a GSH locus can be used to deliver a transgene to skeletal, cardiac or diaphragm muscle, for production of a polypeptide (e.g., an enzyme) or functional RNA (e.g., RNAi, microRNA, antisense RNA) that normally circulates in the blood or for systemic delivery to other tissues to treat, ameliorate, and/or prevent a disorder (e.g., a metabolic disorder, such as diabetes (e.g., insulin), hemophilia (e.g., VIII), a mucopolysaccharide disorder (e.g., Sly syndrome, Hurler Syndrome, Scheie Syndrome, Hurler-Scheie Syndrome, Hunter's Syndrome, Sanfilippo Syndrome A, B, C, D, Morquio Syndrome, Maroteaux-Lamy Syndrome, etc.) or a lysosomal storage disorder (such as Gaucher's disease [glucocerebrosidase], Pompe disease [lysosomal acid .alpha.-glucosidase] or Fabry disease [.alpha.-galactosidase A]) or a glycogen storage disorder (such as Pompe disease [lysosomal acid a glucosidase]). Other suitable proteins for treating, ameliorating, and/or preventing metabolic disorders are described above.

In other embodiments, the ceDNA vector for insertion of a transgene at a GSH locus as disclosed herein can be used to deliver a transgene in a method of treating, ameliorating, and/or preventing a metabolic disorder in a subject in need thereof. Illustrative metabolic disorders and transgenes encoding polypeptides are described herein. Optionally, the polypeptide is secreted (e.g., a polypeptide that is a secreted polypeptide in its native state or that has been engineered to be secreted, for example, by operable association with a secretory signal sequence as is known in the art).

Another aspect of the invention relates to a method of treating, ameliorating, and/or preventing congenital heart failure or PAD in a subject in need thereof, the method comprising administering a ceDNA vector for insertion of a transgene at a GSH locus as described herein to a mammalian subject, wherein the ceDNA vector comprises a transgene encoding, for example, a sarcoplasmic endoreticulum Ca²⁺-ATPase (SERCA2a), an angiogenic factor, phosphatase inhibitor I (I-1), RNAi against phospholamban; a phospholamban inhibitory or dominant-negative molecule such as phospholamban S16E, a zinc finger protein that regulates the phospholamban gene, β2-adrenergic receptor, .beta.2-adrenergic receptor kinase (BARK), PI3 kinase, calsarcan, a .beta.-adrenergic receptor kinase inhibitor (βARKct), inhibitor 1 of protein phosphatase 1, S100A1, parvalbumin, adenylyl cyclase type 6, a molecule that effects G-protein coupled receptor kinase type 2 knockdown such as a truncated constitutively active βARKct, Pim-1, PGC-1α, SOD-1, SOD-2, EC-SOD, kallikrein, HIF, thymosin-β4, mir-1, mir-133, mir-206 and/or mir-208.

The ceDNA vectors as disclosed herein can be administered to the lungs of a subject by any suitable means, optionally by administering an aerosol suspension of respirable particles comprising the ceDNA vectors, which the subject inhales. The respirable particles can be liquid or solid. Aerosols of liquid particles comprising the ceDNA vectors may be produced by any suitable means, such as with a pressure-driven aerosol nebulizer or an ultrasonic nebulizer, as is known to those of skill in the art. See, e.g., U.S. Pat. No. 4,501,729. Aerosols of solid particles comprising the ceDNA vectors may likewise be produced with any solid particulate medicament aerosol generator, by techniques known in the pharmaceutical art.

In some embodiments, the ceDNA vectors can be administered to tissues of the CNS (e.g., brain, eye). In particular embodiments, the ceDNA vectors as disclosed herein may be administered to treat, ameliorate, or prevent diseases of the CNS, including genetic disorders, neurodegenerative disorders, psychiatric disorders and tumors. Illustrative diseases of the CNS include, but are not limited to Alzheimer's disease, Parkinson's disease, Huntington's disease, Canavan disease, Leigh's disease, Refsum disease, Tourette syndrome, primary lateral sclerosis, amyotrophic lateral sclerosis, progressive muscular atrophy, Pick's disease, muscular dystrophy, multiple sclerosis, myasthenia gravis, Binswanger's disease, trauma due to spinal cord or head injury, Tay Sachs disease, Lesch-Nyan disease, epilepsy, cerebral infarcts, psychiatric disorders including mood disorders (e.g., depression, bipolar affective disorder, persistent affective disorder, secondary mood disorder), schizophrenia, drug dependency (e.g., alcoholism and other substance dependencies), neuroses (e.g., anxiety, obsessional disorder, somatoform disorder, dissociative disorder, grief, post-partum depression), psychosis (e.g., hallucinations and delusions), dementia, paranoia, attention deficit disorder, psychosexual disorders, sleeping disorders, pain disorders, eating or weight disorders (e.g., obesity, cachexia, anorexia nervosa, and bulemia) and cancers and tumors (e.g., pituitary tumors) of the CNS.

Ocular disorders that may be treated, ameliorated, or prevented with the ceDNA vectors of the invention include ophthalmic disorders involving the retina, posterior tract, and optic nerve (e.g., retinitis pigmentosa, diabetic retinopathy and other retinal degenerative diseases, uveitis, age-related macular degeneration, glaucoma). Many ophthalmic diseases and disorders are associated with one or more of three types of indications: (1) angiogenesis, (2) inflammation, and (3) degeneration. In some embodiments, the ceDNA vector for insertion of a transgene at a GSH locus as disclosed herein can be employed to deliver anti-angiogenic factors; anti-inflammatory factors; factors that retard cell degeneration, promote cell sparing, or promote cell growth and combinations of the foregoing. Diabetic retinopathy, for example, is characterized by angiogenesis. Diabetic retinopathy can be treated by delivering one or more anti-angiogenic factors either intraocularly (e.g., in the vitreous) or periocularly (e.g., in the sub-Tenon's region). One or more neurotrophic factors may also be co-delivered, either intraocularly (e.g., intravitreally) or periocularly. Additional ocular diseases that may be treated, ameliorated, or prevented with the ceDNA vectors of the invention include geographic atrophy, vascular or “wet” macular degeneration, Stargardt disease, Leber Congenital Amaurosis (LCA), Usher syndrome, pseudoxanthoma elasticum (PXE), x-linked retinitis pigmentosa (XLRP), x-linked retinoschisis (XLRS), Choroideremia, Leber hereditary optic neuropathy (LHON), Archomatopsia, cone-rod dystrophy, Fuchs endothelial corneal dystrophy, diabetic macular edema and ocular cancer and tumors.

In some embodiments, inflammatory ocular diseases or disorders (e.g., uveitis) can be treated, ameliorated, or prevented by the ceDNA vectors of the invention. One or more anti-inflammatory factors can be expressed by intraocular (e.g., vitreous or anterior chamber) administration of the ceDNA vector for insertion of a transgene at a GSH locus as disclosed herein. In other embodiments, ocular diseases or disorders characterized by retinal degeneration (e.g., retinitis pigmentosa) can be treated, ameliorated, or prevented by the ceDNA vectors of the invention. intraocular (e.g., vitreal administration) of the ceDNA vector as disclosed herein encoding one or more neurotrophic factors can be used to treat such retinal degeneration-based diseases. In some embodiments, diseases or disorders that involve both angiogenesis and retinal degeneration (e.g., age-related macular degeneration) can be treated with the ceDNA vectors of the invention. Age-related macular degeneration can be treated by administering the ceDNA vector as disclosed herein encoding one or more neurotrophic factors intraocularly (e.g., vitreous) and/or one or more anti-angiogenic factors intraocularly or periocularly (e.g., in the sub-Tenon's region). Glaucoma is characterized by increased ocular pressure and loss of retinal ganglion cells. Treatments for glaucoma include administration of one or more neuroprotective agents that protect cells from excitotoxic damage using the ceDNA vector as disclosed herein. Accordingly, such agents include N-methyl-D-aspartate (NMDA) antagonists, cytokines, and neurotrophic factors, can be delivered intraocularly, optionally intravitreally using the ceDNA vector as disclosed herein.

In other embodiments, the ceDNA vector for insertion of a transgene at a GSH locus as disclosed herein may be used to treat seizures, e.g., to reduce the onset, incidence or severity of seizures. The efficacy of a therapeutic treatment for seizures can be assessed by behavioral (e.g., shaking, ticks of the eye or mouth) and/or electrographic means (most seizures have signature electrographic abnormalities). Thus, the ceDNA vector for insertion of a transgene at a GSH locus as disclosed herein can also be used to treat epilepsy, which is marked by multiple seizures over time. In one representative embodiment, somatostatin (or an active fragment thereof) is administered to the brain using the ceDNA vector as disclosed herein to treat a pituitary tumor. According to this embodiment, the ceDNA vector as disclosed herein encoding somatostatin (or an active fragment thereof) is administered by microinfusion into the pituitary. Likewise, such treatment can be used to treat acromegaly (abnormal growth hormone secretion from the pituitary). The nucleic acid (e.g., GenBank Accession No. J00306) and amino acid (e.g., GenBank Accession No. P01166; contains processed active peptides somatostatin-28 and somatostatin-14) sequences of somatostatins as are known in the art. In particular embodiments, the ceDNA vector can encode a transgene that comprises a secretory signal as described in U.S. Pat. No. 7,071,172.

Another aspect of the invention relates to the use of a ceDNA vector for insertion of a transgene at a GSH locus as described herein to produce antisense RNA, RNAi or other functional RNA (e.g., a ribozyme) for systemic delivery to a subject in vivo. Accordingly, in some embodiments, the ceDNA vector can comprise a transgene that encodes an antisense nucleic acid, a ribozyme (e.g., as described in U.S. Pat. No. 5,877,022), RNAs that affect spliceosome-mediated trans-splicing (see, Puttaraju et al., (1999) Nature Biotech. 17:246; U.S. Pat. Nos. 6,013,487; 6,083,702), interfering RNAs (RNAi) that mediate gene silencing (see, Sharp et al., (2000) Science 287:2431) or other non-translated RNAs, such as “guide” RNAs (Gorman et al., (1998) Proc. Nat. Acad. Sci. USA 95:4929; U.S. Pat. No. 5,869,248 to Yuan et al.), and the like.

In some embodiments, the ceDNA vector for insertion of a transgene at a GSH locus can further also comprise a transgene that encodes a reporter polypeptide (e.g., an enzyme such as Green Fluorescent Protein, or alkaline phosphatase). In some embodiments, a transgene that encodes a reporter protein useful for experimental or diagnostic purposes, is selected from any of: β-lactamase, β-galactosidase (LacZ), alkaline phosphatase, thymidine kinase, green fluorescent protein (GFP), chloramphenicol acetyltransferase (CAT), luciferase, and others well known in the art. In some aspects, ceDNA vectors comprising a transgene encoding a reporter polypeptide may be used for diagnostic purposes or as markers of the ceDNA vector's activity in the subject to which they are administered.

In some embodiments, the ceDNA vector for insertion of a transgene at a GSH locus can comprise a transgene or a heterologous nucleotide sequence that shares homology with, and recombines with a locus on the host chromosome. This approach may be utilized to correct a genetic defect in the host cell.

In some embodiments, the ceDNA vector for insertion of a transgene at a GSH locus can comprise a transgene that can be used to express an immunogenic polypeptide in a subject, e.g., for vaccination. The transgene may encode any immunogen of interest known in the art including, but not limited to, immunogens from human immunodeficiency virus, influenza virus, gag proteins, tumor antigens, cancer antigens, bacterial antigens, viral antigens, and the like.

D. Testing for Successful Gene Expression Using a ceDNA Vector

Assays well known in the art can be used to test the efficiency of gene delivery by a ceDNA vector can be performed in both in vitro and in vivo models. Knock-in or knock-out of a desired transgene by ceDNA can be assessed by one skilled in the art by measuring mRNA and protein levels of the desired transgene (e.g., reverse transcription PCR, western blot analysis, and enzyme-linked immunosorbent assay (ELISA)). Nucleic acid alterations by ceDNA (e.g., point mutations, or deletion of DNA regions) can be assessed by deep sequencing of genomic target DNA. In one embodiment, ceDNA comprises a reporter protein that can be used to assess the expression of the desired transgene, for example by examining the expression of the reporter protein by fluorescence microscopy or a luminescence plate reader. For in vivo applications, protein function assays can be used to test the functionality of a given gene and/or gene product to determine if gene expression has successfully occurred. For example, it is envisioned that a point mutation in the cystic fibrosis transmembrane conductance regulator gene (CFTR) inhibits the capacity of CFTR to move anions (e.g., CO through the anion channel, can be corrected by delivering a functional (i.e., non-mutated) CFTR gene to the subject with a ceDNA vector. Following administration of a ceDNA vector, one skilled in the art can assess the capacity for anions to move through the anion channel to determine if the CFTR gene has been delivered and expressed. One skilled will be able to determine the best test for measuring functionality of a protein in vitro or in vivo.

It is contemplated herein that the effects of gene expression of the transgene from the ceDNA vector in a cell or subject can last for at least 1 month, at least 2 months, at least 3 months, at least four months, at least 5 months, at least six months, at least 10 months, at least 12 months, at least 18 months, at least 2 years, at least 5 years, at least 10 years, at least 20 years, or can be permanent.

In some embodiments, a transgene in the expression cassette, expression construct, or ceDNA vector described herein can be codon optimized for the host cell. As used herein, the term “codon optimized” or “codon optimization” refers to the process of modifying a nucleic acid sequence for enhanced expression in the cells of the vertebrate of interest, e.g., mouse or human (e.g., humanized), by replacing at least one, more than one, or a significant number of codons of the native sequence (e.g., a prokaryotic sequence) with codons that are more frequently or most frequently used in the genes of that vertebrate. Various species exhibit particular bias for certain codons of a particular amino acid. Typically, codon optimization does not alter the amino acid sequence of the original translated protein. Optimized codons can be determined using e.g., Aptagen's Gene Forge® codon optimization and custom gene synthesis platform (Aptagen, Inc.) or another publicly available database.

### XII. Administration

Exemplary modes of administration of the ceDNA vector for insertion of a transgene at a GSH locus disclosed herein includes oral, rectal, transmucosal, intranasal, inhalation (e.g., via an aerosol), buccal (e.g., sublingual), vaginal, intrathecal, intraocular, transdermal, intraendothelial, in utero (or in ovo), parenteral (e.g., intravenous, subcutaneous, intradermal, intracranial, intramuscular [including administration to skeletal, diaphragm and/or cardiac muscle], intrapleural, intracerebral, and intraarticular), topical (e.g., to both skin and mucosal surfaces, including airway surfaces, and transdermal administration), intralymphatic, and the like, as well as direct tissue or organ injection (e.g., to liver, eye, skeletal muscle, cardiac muscle, diaphragm muscle or brain).

Administration of the ceDNA vector for insertion of a transgene at a GSH locus can be to any site in a subject, including, without limitation, a site selected from the group consisting of the brain, a skeletal muscle, a smooth muscle, the heart, the diaphragm, the airway epithelium, the liver, the kidney, the spleen, the pancreas, the skin, and the eye. Administration of the ceDNA vector for insertion of a transgene at a GSH locus can also be to a tumor (e.g., in or near a tumor or a lymph node). The most suitable route in any given case will depend on the nature and severity of the condition being treated, ameliorated, and/or prevented and on the nature of the particular ceDNA vector that is being used. Additionally, ceDNA permits one to administer more than one transgene in a single vector, or multiple ceDNA vectors (e.g. a ceDNA cocktail).

Administration of the ceDNA vector for insertion of a transgene at a GSH locus disclosed herein to skeletal muscle according to the present invention includes but is not limited to administration to skeletal muscle in the limbs (e.g., upper arm, lower arm, upper leg, and/or lower leg), back, neck, head (e.g., tongue), thorax, abdomen, pelvis/perineum, and/or digits. The ceDNA as disclosed herein vector can be delivered to skeletal muscle by intravenous administration, intra-arterial administration, intraperitoneal administration, limb perfusion, (optionally, isolated limb perfusion of a leg and/or arm; see, e.g. Arruda et al., (2005) Blood 105: 3458-3464), and/or direct intramuscular injection. In particular embodiments, the ceDNA vector as disclosed herein is administered to a limb (arm and/or leg) of a subject (e.g., a subject with muscular dystrophy such as DMD) by limb perfusion, optionally isolated limb perfusion (e.g., by intravenous or intra-articular administration. In certain embodiments, the ceDNA vector for insertion of a transgene at a GSH locus as disclosed herein can be administered without employing “hydrodynamic” techniques.

Administration of the ceDNA vector for insertion of a transgene at a GSH locus as disclosed herein to cardiac muscle includes administration to the left atrium, right atrium, left ventricle, right ventricle and/or septum. The ceDNA vector as described herein can be delivered to cardiac muscle by intravenous administration, intra-arterial administration such as intra-aortic administration, direct cardiac injection (e.g., into left atrium, right atrium, left ventricle, right ventricle), and/or coronary artery perfusion. Administration to diaphragm muscle can be by any suitable method including intravenous administration, intra-arterial administration, and/or intra-peritoneal administration. Administration to smooth muscle can be by any suitable method including intravenous administration, intra-arterial administration, and/or intra-peritoneal administration. In one embodiment, administration can be to endothelial cells present in, near, and/or on smooth muscle.

In some embodiments, a ceDNA vector for insertion of a transgene at a GSH locus according to the present invention is administered to skeletal muscle, diaphragm muscle and/or cardiac muscle (e.g., to treat, ameliorate and/or prevent muscular dystrophy or heart disease (e.g., PAD or congestive heart failure).

### A. Ex Vivo Treatment

In some embodiments, cells are removed from a subject, a ceDNA vector is introduced therein, and the cells are then replaced back into the subject. Methods of removing cells from subject for treatment ex vivo, followed by introduction back into the subject are known in the art (see, e.g., U.S. Pat. No. 5,399,346; the disclosure of which is incorporated herein in its entirety). Alternatively, a ceDNA vector is introduced into cells from another subject, into cultured cells, or into cells from any other suitable source, and the cells are administered to a subject in need thereof.

Cells transduced with a ceDNA vector are preferably administered to the subject in a “therapeutically-effective amount” in combination with a pharmaceutical carrier. Those skilled in the art will appreciate that the therapeutic effects need not be complete or curative, as long as some benefit is provided to the subject.

In some embodiments, the ceDNA vector for insertion of a transgene at a GSH locus can encode a transgene (sometimes called a heterologous nucleotide sequence) that is any polypeptide that is desirably produced in a cell in vitro, ex vivo, or in vivo. For example, in contrast to the use of the ceDNA vectors in a method of treatment as discussed herein, in some embodiments the ceDNA vectors may be introduced into cultured cells and the expressed gene product isolated therefrom, e.g., for the production of antigens or vaccines.

The ceDNA vectors can be used in both veterinary and medical applications. Suitable subjects for ex vivo gene delivery methods as described above include both avians (e.g., chickens, ducks, geese, quail, turkeys and pheasants) and mammals (e.g., humans, bovines, ovines, caprines, equines, felines, canines, and lagomorphs), with mammals being preferred. Human subjects are most preferred. Human subjects include neonates, infants, juveniles, and adults.

One aspect of the technology described herein relates to a method of delivering a transgene to a cell. Typically, for in vitro methods, the ceDNA vector for insertion of a transgene at a GSH locus may be introduced into the cell using the methods as disclosed herein, as well as other methods known in the art. ceDNA vectors disclosed herein are preferably administered to the cell in a biologically-effective amount. If the ceDNA vector is administered to a cell in vivo (e.g., to a subject), a biologically-effective amount of the ceDNA vector is an amount that is sufficient to result in transduction and expression of the transgene in a target cell.

### B. Unit Dosage Forms

In some embodiments, the pharmaceutical compositions can conveniently be presented in unit dosage form. A unit dosage form will typically be adapted to one or more specific routes of administration of the pharmaceutical composition. In some embodiments, the unit dosage form is adapted for administration by inhalation. In some embodiments, the unit dosage form is adapted for administration by a vaporizer. In some embodiments, the unit dosage form is adapted for administration by a nebulizer. In some embodiments, the unit dosage form is adapted for administration by an aerosolizer. In some embodiments, the unit dosage form is adapted for oral administration, for buccal administration, or for sublingual administration. In some embodiments, the unit dosage form is adapted for intravenous, intramuscular, or subcutaneous administration. In some embodiments, the unit dosage form is adapted for intrathecal or intracerebroventricular administration. In some embodiments, the pharmaceutical composition is formulated for topical administration. The amount of active ingredient which can be combined with a carrier material to produce a single dosage form will generally be that amount of the compound which produces a therapeutic effect.

### XIII. Various Applications

The compositions and ceDNA vectors provided herein can be used to deliver a transgene for various purposes as described above. In some embodiments, a transgene can encode a protein or be a functional RNA, and in some embodiments, can be a protein or functional RNA that is modified for research purposes, e.g., to create a somatic transgenic animal model harboring one or more mutations or a corrected gene sequence, e.g., to study the function of the target gene. In another example, the transgene encodes a protein or functional RNA to create an animal model of disease.

In some embodiments, the transgene encodes one or more peptides, polypeptides, or proteins, which are useful for the treatment, amelioration, or prevention of disease states in a mammalian subject. The transgene expressed by the ceDNA vector for insertion of a transgene at a GSH locus is administered to a patient in a sufficient amount to treat a disease associated with an abnormal gene sequence, which can result in any one or more of the following: reduced expression, lack of expression or dysfunction of the target gene.

In some embodiments, the ceDNA vectors are envisioned for use in diagnostic and screening methods, whereby a transgene is transiently or stably expressed in a cell culture system, or alternatively, a transgenic animal model.

Another aspect of the technology described herein provides a method of transducing a population of mammalian cells. In an overall and general sense, the method includes at least the step of introducing into one or more cells of the population, a composition that comprises an effective amount of one or more of the ceDNA disclosed herein.

Additionally, the present invention provides compositions, as well as therapeutic and/or diagnostic kits that include one or more of the disclosed ceDNA vectors or ceDNA compositions, formulated with one or more additional ingredients, or prepared with one or more instructions for their use.

A cell to be administered the ceDNA vector for insertion of a transgene at a GSH locus as disclosed herein may be of any type, including but not limited to neural cells (including cells of the peripheral and central nervous systems, in particular, brain cells), lung cells, retinal cells, epithelial cells (e.g., gut and respiratory epithelial cells), muscle cells, dendritic cells, pancreatic cells (including islet cells), hepatic cells, myocardial cells, bone cells (e.g., bone marrow stem cells), hematopoietic stem cells, spleen cells, keratinocytes, fibroblasts, endothelial cells, prostate cells, germ cells, and the like. Alternatively, the cell may be any progenitor cell. As a further alternative, the cell can be a stem cell (e.g., neural stem cell, liver stem cell). As still a further alternative, the cell may be a cancer or tumor cell. Moreover, the cells can be from any species of origin, as indicated above.

In some embodiments, a nucleic acid of interest for use in the ceDNA vector as disclosed herein can be used to restore the expression of genes that are reduced in expression, silenced, or otherwise dysfunctional in a subject (e.g., a tumor suppressor that has been silenced in a subject having cancer). A nucleic acid of interest for use in the ceDNA vector as disclosed herein can also be used to knockdown the expression of genes that are aberrantly expressed in a subject (e.g., an oncogene that is expressed in a subject having cancer). In some embodiments, a heterologous nucleic acid insert encoding a gene product associated with cancer (e.g., tumor suppressors) may be used to treat the cancer, by administering nucleic acid comprising the heterologous nucleic acid insert to a subject having the cancer. In some embodiments, a nucleic acid of interest as defined herein encodes a small interfering nucleic acid (e.g., shRNAs, miRNAs) that inhibits the expression of a gene product associated with cancer (e.g., oncogenes) may be used to treat the cancer. In some embodiments, a nucleic acid of interest as defined herein encodes a gene product associated with cancer (or a functional RNA that inhibits the expression of a gene associated with cancer) for use, e.g., for research purposes, e.g., to study the cancer or to identify therapeutics that treat the cancer.

A skilled artisan will also realize that the nucleic acids of interest can encode proteins or polypeptides, and that mutations that results in conservative amino acid substitutions may be made in a transgene to provide functionally equivalent variants, or homologs of a protein or polypeptide. In some aspects the disclosure embraces sequence alterations that result in conservative amino acid substitution of a transgene. In some embodiments, a nucleic acid of interest as defined herein encodes a gene having a dominant negative mutation. For example, a nucleic acid of interest as defined herein encodes a mutant protein that interacts with the same elements as a wild-type protein, and thereby blocks some aspect of the function of the wild-type protein.

In some embodiments, the nucleic acid of interest as disclosed herein also include miRNAs. miRNAs and other small interfering nucleic acids regulate gene expression via target RNA transcript cleavage/degradation or translational repression of the target messenger RNA (mRNA). miRNAs are natively expressed, typically as final 19-25 non-translated RNA products. miRNAs exhibit their activity through sequence-specific interactions with the 3′ untranslated regions (UTR) of target mRNAs. These endogenously expressed miRNAs form hairpin precursors which are subsequently processed into a miRNA duplex, and further into a “mature” single stranded miRNA molecule. This mature miRNA guides a multiprotein complex, miRISC, which identifies target site, e.g., in the 3′ UTR regions, of target mRNAs based upon their complementarity to the mature miRNA.

FIG. 7 discloses a non-limiting list of miRNA genes, and their homologues, are useful as transgenes or as targets for small interfering nucleic acids encoded by transgenes (e.g., miRNA sponges, antisense oligonucleotides, TuD RNAs) in certain embodiments of the methods. A miRNA inhibits the function of the mRNAs it targets and, as a result, inhibits expression of the polypeptides encoded by the mRNAs. Thus, blocking (partially or totally) the activity of the miRNA (e.g., silencing the miRNA) can effectively induce, or restore, expression of a polypeptide whose expression is inhibited (derepress the polypeptide). In one embodiment, derepression of polypeptides encoded by mRNA targets of a miRNA is accomplished by inhibiting the miRNA activity in cells through any one of a variety of methods. For example, blocking the activity of a miRNA can be accomplished by hybridization with a small interfering nucleic acid (e.g., antisense oligonucleotide, miRNA sponge, TuD RNA) that is complementary, or substantially complementary to, the miRNA, thereby blocking interaction of the miRNA with its target mRNA. As used herein, an small interfering nucleic acid that is substantially complementary to a miRNA is one that is capable of hybridizing with a miRNA, and blocking the miRNA's activity. In some embodiments, an small interfering nucleic acid that is substantially complementary to a miRNA is an small interfering nucleic acid that is complementary with the miRNA at all but 1, 2, 3, 4, 5, 6, 7, 8, 9, 10, 11, 12, 13, 14, 15, 16, 17, or 18 bases. In some embodiments, an small interfering nucleic acid sequence that is substantially complementary to a miRNA, is an small interfering nucleic acid sequence that is complementary with the miRNA at, at least, one base.

A “miRNA Inhibitor” is an agent that blocks miRNA function, expression and/or processing. For instance, these molecules include but are not limited to microRNA specific antisense, microRNA sponges, tough decoy RNAs (TuD RNAs) and microRNA oligonucleotides (double-stranded, hairpin, short oligonucleotides) that inhibit miRNA interaction with a Drosha complex. MicroRNA inhibitors can be expressed in cells from a transgenes of a nucleic acid, as discussed above. MicroRNA sponges specifically inhibit miRNAs through a complementary heptameric seed sequence (Ebert, M. S. Nature Methods, Epub Aug. 12, 2007). In some embodiments, an entire family of miRNAs can be silenced using a single sponge sequence. TuD RNAs achieve efficient and long-term-suppression of specific miRNAs in mammalian cells (See, e.g., Takeshi Haraguchi, et al., Nucleic Acids Research, 2009, Vol. 37, No. 6 e43, the contents of which relating to TuD RNAs are incorporated herein by reference). Other methods for silencing miRNA function (derepression of miRNA targets) in cells will be apparent to one of ordinary skill in the art.

In some embodiments, a ceDNA as described herein can further comprise, located between the restriction site, a suicide gene, operatively linked to an inducible promoter and/or tissue specific promoter. Thus, such a ceDNA can be used to kill cells upon a signal or induce cells to undergo apoptosis or programmed cell death upon a specific and discrete signal. Such a ceDNA comprising a suicide gene can be used as an escape hatch should the gene targeting or gene editing system not function as expected.

Described herein are methods of targeted insertion of any sequence of interest into a cell. In some embodiments, a nucleic acid of interest is a nucleic acid that encodes a gene or groups of genes whose expression is known to be associated with a particular differentiation lineage of a stem cell. Sequences comprising genes involved in cell fate or other markers of stem cell differentiation can also be inserted. For example, a promoterless construct containing such a gene can be inserted into a specified region (locus) such that the endogenous promoter at that locus drives expression of the gene product.

A significant number of genes and their control elements (promoters and enhancers) are known which direct the developmental and lineage-specific expression of endogenous genes. Accordingly, the selection of control element(s) and/or gene products inserted into stem cells will depend on what lineage and what stage of development is of interest. In addition, as more detail is understood on the finer mechanistic distinctions of lineage-specific expression and stem cell differentiation, it can be incorporated into the experimental protocol to fully optimize the system for the efficient isolation of a broad range of desired stem cells.

Any lineage-specific or cell fate regulatory element (e.g. promoter) or cell marker gene can be used in the compositions and methods described herein. Lineage-specific and cell fate genes or markers are well-known to those skilled in the art and can readily be selected to evaluate a particular lineage of interest. Non-limiting examples of include, but not limited to, regulatory elements obtained from genes such as Ang2, Flk1, VEGFR, MHC genes, aP2, GFAP, Otx2 (see, e.g., U.S. Pat. No. 5,639,618), Dlx (Porteus et al. (1991) Neuron 7:221-229), Nix (Price et al. (1991) Nature 351:748-751), Emx (Simeone et al. (1992) EMBO J. 11:2541-2550), Wnt (Roelink and Nuse (1991) Genes Dev. 5:381-388), En (McMahon et al.), Hox (Chisaka et al. (1991) Nature 350:473-479), acetylcholine receptor beta chain (ACHRβ) (Otl et al. (1994) J. Cell. Biochem. Supplement 18A: 177). Other examples of lineage-specific genes from which regulatory elements can be obtained are available on the NCBI-GEO web site which is easily accessible via the Internet and well known to those skilled in the art.

In certain embodiments, genomic modifications (e.g., transgene integration) at a GSH locus identified herein allow integration of a nucleic acid of interest that may either utilize the promoter found at that safe harbor locus, or allow the expressional regulation of the transgene by an exogenous promoter or control element, as described herein, that is fused to the nucleic acid of interest prior to insertion. An exogenous nucleic acid of interest (i.e., in some embodiments, a target gene or transgene sequence) can comprise, for example, one or more genes or cDNA molecules, or any type of coding or noncoding sequence, as well as one or more control elements (e.g., promoters). In addition, the exogenous nucleic acid sequence may produce one or more RNA molecules (e.g., small hairpin RNAs (shRNAs), inhibitory RNAs (RNAis), microRNAs (miRNAs), etc.). The exogenous nucleic acid sequence is introduced into the cell such that it is integrated into the genome of the cell at a GSH locus identified according to the methods as disclosed herein, or at a GSH loci listed in Table 1A or 1B.

### A. Kits

Another aspect of the technology described herein relates to kits, e.g., kits for insertion of a gene or nucleic acid sequence into a target GSH identified according to the methods as disclosed herein, as well as primer sets to determine integration of the gene or nucleic acid sequence.

In some embodiment, the kit comprises: (a) a ceDNA vector composition as described herein, and primer pairs to determine integration by homologous recombination of nucleic acid located between the restriction site located between the 3′ GSH-specific homology arm and the 5′ GSH-specific homology arm of the ceDNA. In some embodiments, the kit comprises primer pairs that span the site of integration, where the primer pair comprises at least a GSH 5′ primer and at least one GSH 3′ primer, wherein the GSH is identified according to the methods as disclosed herein, wherein the at least one GSH 5′ primer binds to a region of the GSH upstream of the site of integration, and the at least one GSH 3′ primer is at least binds to a region of the GSH downstream of the site of integration. Such primer pairs can function to act as a negative control and do produce a short PCR product when no integration has occurred, and produce no, or a long PCR product incorporating the inserted nucleic acid when nucleic acid insertion has occurred.

In some embodiments, the kit can comprise (a) a GSH-specific single guide and an RNA guided nucleic acid sequence comprised in one or more GSH ceDNA vectors; and (b) GSH knock-in vector comprising GSH ceDNA vector wherein one or more of the sequences of (a) or (b) are comprised on a ceDNA vector as described herein. In some embodiments, the GSH ceDNA vector is a GSH-CRISPR-Cas vector or other GSH-gene editing vector as comprising a gene editing gene as described herein. In some embodiments, the GSH CRISPR-Cas ceDNA vector comprises a GSH-sgRNA nucleic acid sequence and Cas9 nucleic acid sequence.

In another embodiment, the kit can further comprise a GSH knockin donor ceDNA vector comprising a GSH 5′ homology arm and a GSH 3′ homology arm, wherein the GSH 5′ homology arm and the GSH 3′ homology arm are at least 65% complementary to a sequence in the genomic safe harbor (GSH) identified according to the methods as disclosed herein, and where the GSH 5′ and 3′ homology arms allow (i.e., guide) insertion, by homologous recombination, of the nucleic acid sequence located between the GSH 5′ homology arm and a GSH 3′ homology arm into a locus located within the genomic safe harbor. In some embodiments, the GSH Cas9 knockin donor ceDNA vector is a PAX5 Cas9 knockin donor ceDNA vector comprising a PAX5 5′ homology arm and a PAX5 3′ homology arm, wherein the PAX5 5′ homology arm and the PAX5 3′ homology arm are at least 65% complementary to the PAX5 genomic safe harbor locus, and wherein the PAX5 5′ and 3′ homology arms guide insertion, by homologous recombination, of the nucleic acid located between the GSH 5′ homology arm and a GSH 3′ homology arm into a locus within the PAX5 genomic safe harbor.

In some embodiments, the kit comprises a GSH ceDNA vector which is GSH Cas9 knock in ceDNA donor vector.

In some embodiments, the kit further comprising at least one GSH 5′ primer and at least one GSH 3′ primer, wherein the at least one GSH 5′ primer is at least 80% complementary to a region of the GSH upstream of the site of integration, and the at least one GSH 3′ primer is at least 80% complementary to a region of the GSH downstream of the site of integration.

In some embodiments, the kit can comprise two primer pairs, each primer pair functioning as a positive control. For example, in some embodiments, the kit comprises (a) at least two GSH 5′ primers comprising a forward GSH 5′ primer that binds to a region of the GSH upstream of the site of integration, and a reverse GSH 5′ primer that binds to a sequence in the nucleic acid inserted at the site of integration in the GSH sequence, and (b) at least two GSH 3′ primers comprising a forward GSH 3′ primer that binds to a sequence located at the 3′ end of the nucleic acid inserted at the site of integration in the GSH sequence, and a reverse GSH 3′ primer binds to a region of the GSH downstream of the site of integration. In such an embodiment, the primer pairs can function to act as a positive and produce a PCR product only when integration has occurred, and no PCR product is produced when integration has not occurred.

In some embodiments, the kit can comprise at least two GSH 5′ primers comprising;

a forward GSH 5′ primer that is at least 80% complementary to a region of the GSH u-stream of the site of integration, and a reverse GSH 5′ primer that is at least 80% complementary to a sequence in the nucleic acid inserted at the site of integration in the GSH sequence.

In some embodiments, the kit can further comprise at least two GSH 3′ primers comprising; a forward GSH 3′ primer that is at least 80% complementary to a sequence located at the 3′ end of the nucleic acid inserted at the site of integration in the GSH sequence, and a reverse GSH 3′ primer that is at least 80% complementary to a region of the GSH down-stream of the site of integration.

In some embodiments, the kits as disclosed herein can comprise a GSH 5′ primer which is a PAX5 5′ primer and a GSH 3′ primer which is a PAX5 3′ primer, wherein the PAX5 5′ primer and the PAX5 3′ primer flank the site of integration in the PAX5 genomic safe harbor.

### B. Transgenic Animal Models and Modified Cell Lines

Another aspect of the technology described herein relates to a transgenic animal, such as a transgenic mice strain generated using a ceDNA vector as described herein with nucleic acid of interest inserted into a GSH identified according to the methods as disclosed herein.

In some embodiments, one aspect of the invention relates to a transgenic mouse comprising a nucleic acid of interest, such as but not limited to, a nucleic acid encoding a marker gene, therapeutic protein or inserted into the genomic DNA of the mouse at a GSH locus identified according to the methods disclosed herein, where the reporter gene is flanked by lox sites, e.g., LoxP sites. In some embodiments, the GSH locus is located in the genomic DNA of the host animal, e.g., mouse in any of the genes selected from Table 1A or Table 1B. In some embodiments, the GSH locus is located in the intronic or untranslated region (e.g., 3′UTR, 5′UTR exonic) nucleic acid sequence of the PAX5 gene.

Another aspect of the invention as disclosed herein relates to a method of generating a genetically modified animal, such as, e.g., a transgenic mouse, comprising a nucleic acid interest inserted at a Genomic Safe Harbor (GSH) identified according to the methods disclosed herein, where the method comprises a) introducing into a host cell a ceDNA as disclosed herein, and b) introducing the cell into a carrier animal to produce a genetically modified animal. In some embodiments, the host cell is a zygote or a pluripotent stem cell.

ceDNA vectors as described herein can also be administered directly to an organism for transduction of cells in vivo. Administration is by any of the routes normally used for introducing a molecule into ultimate contact with blood or tissue cells including, but not limited to, injection, infusion, topical application and electroporation. Suitable methods of administering such nucleic acids are available and well known to those of skill in the art, and, although more than one route can be used to administer a particular composition, a particular route can often provide a more immediate and more effective reaction than another route.

Methods for introduction of a nucleic acid vector ceDNA vector as disclosed herein can be delivered into hematopoietic stem cells, for example, by the methods as described, for example, in U.S. Pat. No. 5,928,638.

The ceDNA vector compositions as disclosed herein can be used for ex vivo cell transfection for diagnostics, research, or for gene therapy (e.g., via re-infusion of the transfected cells into the host organism). In some embodiments, cells are isolated from the subject organism, transfected with a ceDNA vector as disclosed herein, and re-infused back into the subject organism (e.g., patient or subject). Various cell types suitable for ex vivo transfection are well known to those of skill in the art (see, e.g., Freshney et al., Culture of Animal Cells, A Manual of Basic Technique (3rd ed. 1994)) and the references cited therein for a discussion of how to isolate and culture cells from patients).

In one embodiment, stem cells are used in ex vivo procedures for cell transfection and gene therapy. The advantage to using stem cells is that they can be differentiated into other cell types in vitro, or can be introduced into a mammal (such as the donor of the cells) where they will engraft in the bone marrow. Methods for differentiating CD34+ cells in vitro into clinically important immune cell types using cytokines such a GM-CSF, IFN-γ and TNF-α are known (see Inaba et al., J. Exp. Med. 176:1693-1702 (1992)).

Stem cells are isolated for transduction and differentiation using known methods. For example, stem cells are isolated from bone marrow cells by panning the bone marrow cells with antibodies which bind unwanted cells, such as CD4+ and CD8+ (T cells), CD45+ (panb cells), GR-1 (granulocytes), and lad (differentiated antigen presenting cells) (see Inaba et al., J. Exp. Med. 176:1693-1702 (1992)). In one embodiment, the cell to be used is an oocyte. In other embodiments, cells derived from model organisms may be used. These can include cells derived from xenopus, insect cells (e.g., drosophilia) and nematode cells.

Some embodiments of the technology described herein can be defined according to any of the following numbered paragraphs:
134. A close-ended DNA (ceDNA) nucleic acid vector comprising, in the following order:
   a. a terminal repeat (TR), e.g., ITR
   b. at least a portion of the genomic safe harbor (GSH) nucleic acid identified as a genomic safe harbor in the method of any of paragraphs 41-51, and
   c. a terminal repeat (TR), e.g., ITR.
135. The ceDNA vector composition of paragraph 1, wherein the at least a portion of the GSH nucleic acid comprises the PAX5 genomic DNA or a fragment thereof
136. The ceDNA vector composition of paragraph 1, wherein the GSH nucleic acid comprises an untranslated sequence or an intron of the PAX5 gene.
137. The ceDNA vector composition of paragraph 1, wherein the GSH nucleic acid is a nucleic acid selected from any of the nucleic acid sequences listed in Table 1A or 1B.
138. The ceDNA vector composition of paragraph 1, wherein the at least portion of the GSH comprises at least one modification as compared to the wild-type GSH sequence.
139. The ceDNA vector composition of paragraph 5, wherein the modification is a nucleic acid sequence comprising a restriction cloning site.
140. The ceDNA vector composition of paragraph 5, wherein the modification is a nucleic acid sequence comprising one or more target sites for one or more nucleases.
141. The ceDNA vector composition of paragraph 7, wherein the nuclease is selected from a zinc finger nuclease (ZFN), a TAL-effector domain nuclease (TALEN), or a CRISPR/Cas system.
142. The ceDNA vector composition of any of paragraphs 1-8, wherein the portion of GSH nucleic acid is at least 1 kb in length.
143. The ceDNA vector composition of any of paragraphs 1-8, wherein the portion of GSH nucleic acid is between 300-3 kb in length.
144. The ceDNA vector composition of any of paragraphs 1-8, wherein the portion of the GSH is a target site for a guide RNA (gRNA).
145. The ceDNA vector composition of any of paragraphs 11, wherein the gRNA is for a sequence-specific nuclease selected from any of: a TAL-nuclease, a zinc-finger nuclease (ZFN), a meganuclease, a megaTAL, or an RNA guide endonuclease (e.g., CAS9, cpf1, nCAS9).
146. The ceDNA vector composition of any of paragraphs 11-12, wherein one or more of the terminal repeat (TR) are inverted TRs (ITRs).
147. The ceDNA vector composition of any of paragraphs 11-13, wherein at least one of the terminal repeat (TR) is a modified terminal repeat.
148. The ceDNA vector composition of any of paragraphs 11-14, wherein the vector is single stranded circular DNA under nucleic acid denaturing conditions.
149. A close-ended DNA (ceDNA) nucleic acid vector composition comprising, in the following order:
   a. a terminal repeat (TR), e.g., ITR
   b. a GSH 5′ homology arm,
   c. a nucleic acid sequence comprising a restriction cloning site, and
   d. a GSH 3′ homology arm, and
   e. a terminal repeat (TR), e.g., ITR
   wherein the 5′ homology arm and the 3′ homology arm bind to a target site located in a genomic safe harbor locus identified in the method of any of paragraphs 41 to 51, and wherein the 5′ and 3′ homology arms guide homologous recombination into a locus located within the genomic safe harbor.
150. The ceDNA vector composition of paragraph 16, wherein the 5′ and 3′ homology arms are between 30-2000 bp in length.
151. The ceDNA vector composition of paragraphs 16 or 17, further comprising, inserted at the restriction cloning site, at least one or more of the following:
   a. a gene editing nucleic acid sequence,
   b. a target site for one or more nucleases;
   c. a nucleic acid of interest,
   d. a guide RNA (gRNA) for a RNA-guided DNA endonuclease.
152. The ceDNA vector composition of paragraph 18, wherein the gene editing nucleic acid sequence encodes a gene editing nucleic acid molecule selected from the group consisting of: a sequence-specific nuclease, one or more guide RNA (gRNA), CRISPR/Cas, a ribonucleoprotein (RNP) or any combination thereof
153. The ceDNA vector composition of paragraph 19, wherein the sequence-specific nuclease comprises: a TAL-nuclease, a zinc-finger nuclease (ZFN), a meganuclease, a megaTAL, or an RNA guide endonuclease (e.g., CAS9, cpf1, nCAS9).
154. The ceDNA vector composition of paragraph 18, wherein the nucleic acid of interest is a miRNA, RNAi, encodes a therapeutic protein, antibody, peptide, suicide gene, apoptosis gene or any gene or combination of genes listed in Table 3.
155. The ceDNA vector composition of paragraph 21, further comprising a control element, promoter or regulatory element operatively linked to the nucleic acid of interest. 156. The ceDNA vector composition of any of paragraphs 16-22, wherein nucleic acid of interest or
gene editing nucleic acid sequence is in an orientation for integration in the GSH in a forward orientation.
157. The ceDNA vector composition of any of paragraphs 16-22, wherein nucleic acid of interest or gene editing nucleic acid sequence is in an orientation for integration in the GSH in a reverse orientation.
158. The ceDNA vector composition of any of paragraphs 16-24, wherein GSH 5′ homology arm and the GSH 3′ homology arm bind to target sites that are spatially distinct nucleic acid sequences in the genomic safe harbor identified in the method of any of paragraphs 41 to 51.
159. The ceDNA vector composition of any of paragraphs 16-25, wherein the GSH 5′ homology arm and the GSH 3′ homology arm are at least 65% complementary to a target sequence in the genomic safe harbor locus identified in the method of any of paragraphs 41 to 51.
160. The ceDNA vector composition of any of paragraphs 16-26, wherein the GSH 5′ homology arm and the 3′ homology arm bind to a target site located in the PAX5 genomic safe harbor sequence.
161. The ceDNA vector composition of any of paragraphs 16-27, wherein the GSH 5′ homology arm and the GSH 3′ homology arm are at least 65% complementary to at least part the PAX5 genomic safe harbor sequence.
162. The ceDNA vector composition of any of paragraphs 16-28, wherein the GSH 5′ homology arm and the GSH 3′ homology arm bind to a GSH of target site located in a gene selected from Table 1.
163. The ceDNA vector composition of any of paragraphs 16-29, wherein one or more of the terminal repeat (TR) are inverted TRs (ITRs).
164. The ceDNA vector composition of any of paragraphs 16-30, wherein at least one of the terminal repeat (TR) is a modified terminal repeat.
165. The ceDNA vector composition of any of paragraphs 16-31, wherein the vector is single stranded circular DNA under nucleic acid denaturing conditions.

166. A cell comprising the ceDNA vector composition of any of paragraphs 1-32.
167. The cell of paragraph 33, wherein the cell is a red blood cell (RBC) or RBC precursor cell.
168. The cell of paragraph 34, wherein the RBC precursor cell is a CD44+ or CD34+ cell.
169. The cell of paragraph 33, wherein the cell is a stem cell.
170. The cell of paragraph 33, wherein the cell is an iPS cell or embryonic stem cell.
171. The cell of paragraph 37, wherein the iPS cell is a patient-derived iPSC.
172. The cell of any of paragraphs 33-38, wherein the cell is a mammalian cell.
173. The cell of paragraph 39, wherein the mammalian cell is a human cell.
174. A method for inserting a nucleic acid of interest or gene editing nucleic acid sequence into a genomic safe harbor (GSH) loci of a cell, the method comprising introducing the ceDNA vector of any of paragraphs 1-32 into the cell, whereby homologous recombination of 3′ and 5′ homology arms with regions of the GSH integrate the nucleic acid sequence or gene editing nucleic acid sequence into the GSH locus.
175. The method of paragraph 42, wherein the nucleic acid sequence is integrated into the GSH in a forward orientation.
176. The method of paragraph 42, wherein the nucleic acid sequence is integrated into the GSH in a reverse orientation.
177. A transgenic organism comprising an integrated nucleic acid of interest or gene editing nucleic acid sequence located in a genomic safe harbor (GSH) locus selected from Table 1A or 1B, wherein integration of the nucleic acid of interest or gene editing nucleic acid sequence into the GSH locus is according to the method of paragraph 42.
178. A kit comprising:
   a. ceDNA vector composition of any of paragraphs 1-32; and
   b. at least one GSH 5′ primer and at least one GSH 3′ primer, wherein the GSH is identified by the method of any of paragraphs 41 to 51, wherein the at least one GSH 5′ primer binds to a region of the GSH upstream of the site of integration, and the at least one GSH 3′ primer is at least binds to a region of the GSH downstream of the site of integration; and/or
      i. at least two GSH 5′ primers comprising a forward GSH 5′ primer that binds to a region of the GSH upstream of the site of integration, and a reverse GSH 5′ primer that binds to a sequence in the nucleic acid inserted at the site of integration in the GSH sequence, wherein the GSH is any of those in Table 1A or 1B;
   c. at least two GSH 3′ primers comprising a forward GSH 3′ primer that binds to a sequence located at the 3′ end of the nucleic acid inserted at the site of integration in the GSH sequence, and a reverse GSH 3′ primer binds to a region of the GSH downstream of the site of integration, and wherein the GSH is any of those in Table 1A or 1B. 179. The kit of paragraph 545 wherein the ceDNA comprises at least one modified terminal repeat.
180. A kit comprising:
   (a) a GSH-specific single guide and an RNA guided nucleic acid sequence comprised in one or more ceDNA vectors; and
   (b) a ceDNA GSH knock-in vector comprising GSH vector,
   wherein one or more of the sequences of (a) or (b) are comprised on a ceDNA vector of any of paragraphs 1-32.
181. The kit of paragraph 47, wherein the GSH vector is a GSH-CRISPR-Cas vector.
182. The kit of paragraph 48, wherein the GSH CRISPR-Cas vector comprises a GSH-sgRNA nucleic acid sequence and Cas9 nucleic acid sequence.
183. The kit of paragraph 48, comprising a GSH knockin donor vector comprising a GSH 5′ homology arm and a GSH 3′ homology arm, wherein the GSH 5′ homology arm and the GSH 3′ homology arm are at least 65% complementary to a sequence in the genomic safe harbor (GSH) shown in Tables 1A or 1B, and wherein the GSH 5′ and 3′ homology arms guide insertion, e.g., by homologous recombination, of the nucleic acid sequence located between the GSH 5′ homology arm and a GSH 3′ homology arm into a locus located within the genomic safe harbor of any of those in Table 1A or 1B.
184. The kit of paragraph 48, wherein the GSH knockin donor vector is a PAX5 knockin donor vector comprising a PAX5 5′ homology arm and a PAX5 3′ homology arm, wherein the PAX5 5′ homology arm and the PAX5 3′ homology arm are at least 65% complementary to the PAX5 genomic safe harbor locus, and wherein the PAX5 5′ and 3′ homology arms guide insertion, by homologous recombination, of the nucleic acid located between the GSH 5′ homology arm and a GSH 3′ homology arm into a locus within the PAX5 genomic safe harbor.
185. The kit of paragraph 48, wherein the GSH knockin donor vector is a knockin donor vector comprising a 5′ homology arm which binds to a GSH locus listed in Table 1A or 1B, and a 3′ homology arm which binds to a spatially distinct region of the same GSH locus that the 5′ homology arm binds to, wherein the 5′ and 3′ homology arms guide insertion, by homologous recombination, of the nucleic acid located between the GSH 5′ homology arm and a GSH 3′ homology arm into a GSH locus listed in Table 1A or 1B.
186. The kit of paragraph 48, wherein the GSH vector is GSH Cas9 knock in donor vector.
187. The kit of any of paragraphs 48-53, further comprising at least one GSH 5′ primer and at least one GSH 3′ primer, wherein the GSH is identified by the method of any of paragraphs 41 to 51, wherein the at least one GSH 5′ primer is at least 80% complementary to a region of the GSH upstream of the site of integration, and the at least one GSH 3′ primer is at least 80% complementary to a region of the GSH downstream of the site of integration.
188. The kit of any of paragraphs 48-54, further comprising at least two GSH 5′ primers comprising;
   a. a forward GSH 5′ primer that is at least 80% complementary to a region of the GSH upstream of the site of integration, and
   b. a reverse GSH 5′ primer that is at least 80% complementary to a sequence in the nucleic acid inserted at the site of integration in the GSH sequence, wherein the GSH is identified by the method of any of paragraphs 41 to 51.
189. The kit of any of paragraphs 48-55, further comprising at least two GSH 3′ primers comprising;
   a. a forward GSH 3′ primer that is at least 80% complementary to a sequence located at the 3′ end of the nucleic acid inserted at the site of integration in the GSH sequence, and
   b. a reverse GSH 3′ primer that is at least 80% complementary to a region of the GSH
   downstream of the site of integration, and wherein the GSH is wherein the GSH is any of those in Table 1A or 1B.
190. The kit of any of paragraphs 58-67, wherein the GSH 5′ primer is a PAX5 5′ primer and the GSH 3′ primer is a PAX 3′ primer, wherein the PAX5 5′ primer and the PAX5 3′ primer flank the site of integration in the PAX5 genomic safe harbor.
191. A transgenic mouse comprising a marker gene inserted into the genomic DNA of the mouse at a GSH locus, wherein the GSH is any of those in Table 1A or 1B, wherein the reporter gene is flanked by lox sites, and wherein the transgenic mice is generated by the method of paragraph 42.
192. The transgenic mice of paragraph 58, wherein the lox sites are LoxP sites.
193. The transgenic mice of paragraph 58, wherein the GSH locus is located in the genomic DNA of any of the genes selected from Table 1A or 1B.
194. The transgenic mice of paragraph 58, wherein the GSH locus is located in the intronic or untranslated region (e.g., 3′UTR, 5′UTR exonic) nucleic acid sequence of the PAX5 gene or Kif6 gene.
195. A method of generating a genetically modified animal comprising a nucleic acid interest inserted at a Genomic Safe Harbor (GSH) listed in Table 1A or 1B, comprising a) introducing into a host cell a ceDNA of any of paragraphs 1-32, and b) introducing the cell generated in (a) into a carrier animal to produce a genetically modified animal.
196. The method of paragraph 63, wherein the host cell is a zygote or a pluripotent stem cell.
197. A genetically modified animal produced by the method of paragraph 62.

### Definitions

Unless otherwise defined herein, scientific and technical terms used in connection with the present application shall have the meanings that are commonly understood by those of ordinary skill in the art to which this disclosure belongs. It should be understood that this invention is not limited to the particular methodology, protocols, and reagents, etc., described herein and as such can vary. The terminology used herein is for the purpose of describing particular embodiments only, and is not intended to limit the scope of the present invention, which is defined solely by the claims. Definitions of common terms in immunology and molecular biology can be found in The Merck Manual of Diagnosis and Therapy, 19th Edition, published by Merck Sharp & Dohme Corp., 2011 (ISBN 978-0-911910-19-3); Robert S. Porter et al. (eds.), Fields Virology, 6^{th} Edition, published by Lippincott Williams & Wilkins, Philadelphia, Pa., USA (2013), Knipe, D. M. and Howley, P. M. (ed.), The Encyclopedia of Molecular Cell Biology and Molecular Medicine, published by Blackwell Science Ltd., 1999-2012 (ISBN 9783527600908); and Robert A. Meyers (ed.), Molecular Biology and Biotechnology: a Comprehensive Desk Reference, published by VCH Publishers, Inc., 1995 (ISBN 1-56081-569-8); Immunology by Werner Luttmann, published by Elsevier, 2006; Janeway's Immunobiology, Kenneth Murphy, Allan Mowat, Casey Weaver (eds.), Taylor & Francis Limited, 2014 (ISBN 0815345305, 9780815345305); Lewin's Genes XI, published by Jones & Bartlett Publishers, 2014 (ISBN-1449659055); Michael Richard Green and Joseph Sambrook, Molecular Cloning: A Laboratory Manual, 4^{th} ed., Cold Spring Harbor Laboratory Press, Cold Spring Harbor, N.Y., USA (2012) (ISBN 1936113414); Davis et al., Basic Methods in Molecular Biology, Elsevier Science Publishing, Inc., New York, USA (2012) (ISBN 044460149X); Laboratory Methods in Enzymology: DNA, Jon Lorsch (ed.) Elsevier, 2013 (ISBN 0124199542); Current Protocols in Molecular Biology (CPMB), Frederick M. Ausubel (ed.), John Wiley and Sons, 2014 (ISBN047150338X, 9780471503385), Current Protocols in Protein Science (CPPS), John E. Coligan (ed.), John Wiley and Sons, Inc., 2005; and Current Protocols in Immunology (CPI) (John E. Coligan, ADA M Kruisbeek, David H Margulies, Ethan M Shevach, Warren Strobe, (eds.) John Wiley and Sons, Inc., 2003 (ISBN 0471142735, 9780471142737), the contents of which are all incorporated by reference herein in their entireties.

As used herein, the terms “heterologous nucleotide sequence” and “transgene” are used interchangeably and refer to a nucleic acid of interest (other than a nucleic acid encoding a capsid polypeptide) that is incorporated into and may be delivered and expressed by a ceDNA vector as disclosed herein.

As used herein, the terms “expression cassette” and “transcription cassette” are used interchangeably and refer to a linear stretch of nucleic acids that includes a transgene that is operably linked to one or more promoters or other regulatory sequences sufficient to direct transcription of the transgene, but which does not comprise capsid-encoding sequences, other vector sequences or inverted terminal repeat regions. An expression cassette may additionally comprise one or more cis-acting sequences (e.g., promoters, enhancers, or repressors), one or more introns, and one or more post-transcriptional regulatory elements.

The term “Genomic Safe Harbor” is also interchangeably referred to herein as “GSH” or “safe harbor gene” or “safe harbor locus” refers to a location within a genome, including a region of genomic DNA or a specific site, that can be used for integrating an exogenous nucleic acid wherein the integration does not cause any significant deleterious effect on the growth of the host cell by the addition of the exogenous nucleic acid alone. That is, a GSH refers to a gene or locus in the genome that a nucleic acid sequence can be inserted such that the sequence can integrate and function in a predictable manner (e.g., express a protein of interest) without significant negative consequences to endogenous gene activity, or the promotion of cancer. For example, a genomic safe harbor (GSHs) is a site in the host cells genome that is able to accommodate the integration of new genetic material in a manner that ensures that the newly inserted genetic elements (i) function predictably and (ii) do not cause significant alterations of the host genome thereby averting a risk to the host cell or organism, and (iii) preferably the inserted nucleic acid is not perturbed by any read-through expression from neighboring genes, and (iv) does not activate nearby genes. GSHs can be a specific site, or can be a region of the genomic DNA. A GSH can be a chromosomal site where transgenes can be stably and reliably expressed in all tissues of interest without adversely affecting endogenous gene structure or expression. In some embodiments, a safe harbor gene is also a locus or gene where an inserted nucleic acid sequence can be expressed efficiently and at higher levels than a non-safe harbor site.

The term “locus” refers to the position in a chromosome of a particular gene, target site of integration, or GSH. The term “loci” is pleural of locus.

The term “GSH loci” is the plural of “locus” and refers to a region of the chromosome of where integration does not cause any significant effect on the growth or differentiation of the target cell by the addition of the nucleic acid alone.

The term “endogenous viral element” or “EVE” is a DNA sequence derived from a virus, and present within the germline of a non-viral organism. EVEs may be entire viral genomes (proviruses), or fragments of viral genomes. They arise when a viral DNA sequence becomes integrated into the genome of a germ cell that goes on to produce a viable organism. The newly established EVE can be inherited from one generation to the next as an allele in the host species, and may even reach fixation.

The term “provirus” refers to the genome of a virus when it is integrated or inserted into a host cell's DNA. Provirus refers to the duplex DNA form of the retroviral genome linked to a cellular chromosome. The provirus is produced by reverse transcription of the RNA genome and subsequent integration into the chromosomal DNA of the host cell.

The term “parvovirus” refers to any species of the family (Parvoviridae) comprising or consisting of DNA virus with linear single-stranded DNA genomes that include the causative agents of fifth disease in humans, panleukopenia in cats, and parvovirus infection in dogs and other carnivore host species.

The term “circovirus” is a genus of DNA-viruses with single-stranded circular genome (family Circoviridae), various species of which cause potentially lethal infections in swine, fowls, pigeons, and psittacine birds.

The term “proto-species” as disclosed herein refers to an ancestral species that gave rise to a group of related species or organisms that may or may not be capable of exchanging genetic information and cross-breeding. The species is the principal natural taxonomic unit, ranking below a genus and denoted by a Latin binomial, e.g., Homo sapiens.

The term “orthologous” refers to genes in different species or organisms derived from a common ancestral gene following speciation from a common ancestral gene. Commonly, orthologues retain the same function in the course of evolution and are genes with similar sequence, however, as the host species evolved, the same gene may have been adapted to perform a different role. For example, piRNA (a crystalline gene of the eye) is a gene that is adapted to perform a different role, has it comprises a complex path of domain proteins. Orthologues in divergent species often have an identical function and in some embodiments, are often interchangeable between species without losing function, for example Metazomes in bacteria. Once a phylogenic tree used to establish phylogenetic relationships between species has been constructed using a program such as CLUSTAL (Thompson et al. (1994) Nucleic Acids Res. 22: 4673-4680; Higgins et al. (1996) supra) potential orthologous sequences can be placed into the phylogenetic tree and their relationship to genes from the species of interest can be determined. Orthologous sequences can also be identified by a reciprocal BLAST strategy. Once an orthologous sequence has been identified, the function of the orthologue can be deduced from the identified function of the reference sequence. Orthologous genes from different organisms have highly conserved functions, and very often essentially identical functions (Lee et al. (2002) Genome Res. 12: 493-502; Remm et al. (2001) J. Mol. Biol. 314: 1041-1052). Paralogous genes, which have diverged through gene duplication, may retain similar functions of the encoded proteins. In such cases, paralogs can be used interchangeably with respect to certain embodiments of the instant invention (for example, transgenic expression of a coding sequence).

The term “taxonomic order” refers to orderly classification of plants and animals according to their presumed natural relationships. Species relatedness, based on analysis of genomic sequence data provides a quantitative alternative approach to the natural relationships deduced from physical relationships.

The term “catacea” refers to the taxonomic (infra)order of aquatic marine mammals comprising among others, baleen whales, toothed whales, dolphins and porpoises, and related forms and that have a torpedo-shaped nearly hairless body, paddle-shaped forelimbs but no hind limbs, one or two nares opening externally at the top of the head, and a horizontally flattened tail used for locomotion.

The term “chiroptera” refers to the taxonomic order of mammals capable of true flight, and comprise bats.

The term “lagomorpha” refers to the taxonomic order of gnawing herbivorous mammals having two pairs of incisors in the upper jaw one behind the other, usually soft fur, and short or rudimentary tail, made up of two families (Leporidae and Ochotonidae genera that comprise Leporidae family) comprising the rabbits, hares, and pikas, and was formerly considered a suborder of the order Rodentia.

The term “Macropodidae” refers to the taxonomic family of diprotodont marsupial mammals comprising the kangaroos, wallabies, and rat kangaroos that are all saltatory animals with long hind limbs and weakly developed forelimbs and are typically inoffensive terrestrial herbivores.

The term “Rodentia” is of the taxonomic order of relatively small gnawing mammals (such as a mouse, squirrel, or beaver) that have in both jaws a single pair of incisors with a chisel-shaped edge. It includes all rodents.

The term “primates” is the taxonomic order of mammals that are characterized especially by advanced development of binocular vision resulting in stereoscopic depth perception, specialization of the hands and feet for grasping, and enlargement of the cerebral hemispheres and include humans, apes, monkeys, and related forms (such as lemurs and tarsiers).

The term “monotremata” refers to the taxonomic order of egg-laying mammals comprising the platypuses and echidnas.

The term “syntenic” refers to similar organization or ordering of a series of genes in different species.

The terms “polynucleotide” and “nucleic acid,” used interchangeably herein, refer to a polymeric form of nucleotides of any length, either ribonucleotides or deoxyribonucleotides. Thus, this term includes single, double, or multi-stranded DNA or RNA, genomic DNA, cDNA, DNA-RNA hy-brids, or a polymer including purine and pyrimidine bases or other natural, chemically or biochemi-cally modified, non-natural, or derivatized nucleotide bases. “Oligonucleotide” generally refers to polynucleotides of between about 5 and about 100 nucleotides of single- or double-stranded DNA. However, for the purposes of this disclosure, there is no upper limit to the length of an oligonucleo-tide. Oligonucleotides are also known as “oligomers” or “oligos” and may be isolated from genes, or chemically synthesized by methods known in the art. The terms “polynucleotide” and “nucleic ac-id” should be understood to include, as applicable to the embodiments being described, single-stranded (such as sense or antisense) and double-stranded polynucleotides.

As used herein, the terms “heterologous nucleotide sequence” and “transgene” are used interchangeably and refer to a nucleic acid of interest (other than a nucleic acid encoding a capsid polypeptide) that is incorporated into and may be delivered and expressed by a ceDNA vector as disclosed herein. Transgenes of interest include, but are not limited to, nucleic acids encoding polypeptides, preferably therapeutic (e.g., for medical, diagnostic, or veterinary uses) or immunogenic polypeptides (e.g., for vaccines). In some embodiments, nucleic acids of interest include nucleic acids that are transcribed into therapeutic RNA. Transgenes included for use in the ceDNA vectors of the invention include, but are not limited to, those that express or encode one or more polypeptides, peptides, ribozymes, aptamers, peptide nucleic acids, siRNAs, RNAis, miRNAs, lncRNAs, antisense oligo- or polynucleotides, antibodies, antigen binding fragments, or any combination thereof. A transgene can be a “genetic medicine” and encompasses any of: an inhibitor, nucleic acid, oligonucleotide, silencing nucleic acid, miRNA, RNAi, antagonist, agonist, polypeptide, peptide, antibody or antibody fragments, fusion proteins, or variants thereof, epitopes, antigens, aptamers, ribosomes, and the like. A transgene used herein in the ceDNA vector is not limited in size.

The term “genetic medicine” as disclosed herein relates to any DNA structure or nucleic acid sequence that can be used to treat or prevent a disease or disorder in a subject.

As used herein, the terms “expression cassette” and “transcription cassette” are used interchangeably and refer to a linear stretch of nucleic acids that includes a transgene that is operably linked to one or more promoters or other regulatory sequences sufficient to direct transcription of the transgene, but which does not comprise capsid-encoding sequences, other vector sequences or inverted terminal repeat regions. An expression cassette may additionally comprise one or more cis-acting sequences (e.g., promoters, enhancers, or repressors), one or more introns, and one or more post-transcriptional regulatory elements.

The terms “polynucleotide” and “nucleic acid,” used interchangeably herein, refer to a polymeric form of nucleotides of any length, either ribonucleotides or deoxyribonucleotides. Thus, this term includes single, double, or multi-stranded DNA or RNA, genomic DNA, cDNA, DNA-RNA hybrids, or a polymer including purine and pyrimidine bases or other natural, chemically or biochemically modified, non-natural, or derivatized nucleotide bases. “Oligonucleotide” generally refers to polynucleotides of between about 5 and about 100 nucleotides of single- or double-stranded DNA. However, for the purposes of this disclosure, there is no upper limit to the length of an oligonucleotide. Oligonucleotides are also known as “oligomers” or “oligos” and may be isolated from genes, or chemically synthesized by methods known in the art. The terms “polynucleotide” and “nucleic acid” should be understood to include, as applicable to the embodiments being described, single-stranded (such as sense or antisense) and double-stranded polynucleotides.

The term “nucleic acid construct” as used herein refers to a nucleic acid molecule, either single- or double-stranded, which is isolated from a naturally occurring gene or which is modified to contain segments of nucleic acids in a manner that would not otherwise exist in nature or which is synthetic. The term nucleic acid construct is synonymous with the term “expression cassette” when the nucleic acid construct contains the control sequences required for expression of a coding sequence of the present disclosure. An “expression cassette” includes a DNA coding sequence operably linked to a promoter.

By “hybridizable” or “complementary” or “substantially complementary” it is meant that a nucleic acid (e.g., RNA) includes a sequence of nucleotides that enables it to non-covalently bind, i.e. form Watson-Crick base pairs and/or G/U base pairs, “anneal”, or “hybridize,” to another nucleic acid in a sequence-specific, antiparallel, manner (i.e., a nucleic acid specifically binds to a complementary nucleic acid) under the appropriate in vitro and/or in vivo conditions of temperature and solution ionic strength. As is known in the art, standard Watson-Crick base-pairing includes: adenine (A) pairing with thymidine (T), adenine (A) pairing with uracil (U), and guanine (G) pairing with cytosine (C). In addition, it is also known in the art that for hybridization between two RNA molecules (e.g., dsRNA), guanine (G) base pairs with uracil (U). For example, G/U base-pairing is partially responsible for the degeneracy (i.e., redundancy) of the genetic code in the context of tRNA anti-codon base-pairing with codons in mRNA. In the context of this disclosure, a guanine (G) of a protein-binding segment (dsRNA duplex) of a subject DNA-targeting RNA molecule is considered complementary to a uracil (U), and vice versa. As such, when a G/U base-pair can be made at a given nucleotide position a protein-binding segment (dsRNA duplex) of a subject DNA-targeting RNA molecule, the position is not considered to be non-complementary, but is instead considered to be complementary.

The terms “peptide,” “polypeptide,” and “protein” are used interchangeably herein, and refer to a polymeric form of amino acids of any length, which can include coded and non-coded amino acids, chemically or biochemically modified or derivatized amino acids, and polypeptides having modified peptide backbones.

A DNA sequence that “encodes” a particular RNA or protein gene product is a DNA nucleic acid sequence that is transcribed into the particular RNA and/or protein. A DNA polynucleotide may encode an RNA (mRNA) that is translated into protein, or a DNA polynucleotide may encode an RNA that is not translated into protein (e.g., tRNA, rRNA, or a DNA-targeting RNA; also called “non-coding” RNA or “ncRNA”).

As used herein, the term “gene editing molecule” refers to one or more of a protein or a nucleic acid encoding for a protein, wherein the protein is selected from the group comprising a transposase, a nuclease, an integrase, a guide RNA (gRNA), a guide DNA, a ribonucleoprotein (RNP), or an activator RNA. A nuclease gene editing molecule is a protein having nuclease activity, with nonlimiting examples including: a CRISPR protein (Cas), CRISPR associated protein 9 (Cas9); a type IIS restriction enzyme; a transcription activator-like effector nuclease (TALEN); and a zinc finger nuclease (ZFN), a meganuclease, engineered site-specific nucleases or deactivated CAS for CRISPRi or CRISPRa systems. The gene editing molecule can also comprise a DNA-binding domain and a nuclease. In certain embodiments, the gene editing molecule comprises a DNA-binding domain and a nuclease. In certain embodiments, the DNA-binding domain comprises a guide RNA. In certain embodiments, the DNA-binding domain comprises a DNA-binding domain of a TALEN. In certain embodiments at least one gene editing molecule comprises one or more transposable element(s). In certain embodiments, the one or more transposable element(s) comprise a circular DNA. In certain embodiments, the one or more transposable element(s) comprise a plasmid vector or a minicircle DNA vector. In certain embodiments, the DNA-binding domain comprises a DNA-binding domain of a zinc-finger nuclease. In certain embodiments at least one gene editing molecule comprises one or more transposable element(s). In certain embodiments, the one or more transposable element(s) comprise a linear DNA. The linear recombinant and non-naturally occurring DNA sequence encoding a transposon may be produced in vitro. Linear recombinant and non-naturally occurring DNA sequences of the disclosure may be a product of restriction digest of a circular DNA. In certain embodiments, the circular DNA is a plasmid vector or a minicircle DNA vector. Linear recombinant and non-naturally occurring DNA sequences of the disclosure may be a product of a polymerase chain reaction (PCR). Linear recombinant and non-naturally occurring DNA sequences of the disclosure may be a double-stranded Doggybone™ DNA sequence. Doggybone™ DNA sequences of the disclosure may be produced by an enzymatic process that solely encodes an antigen expression cassette, comprising antigen, promoter, poly-A tail and telomeric ends.

As used herein, the term “gene editing functionality” refers to the insertion, deletion or replacement of DNA at a specific site in the genome with a loss or gain of function. The insertion, deletion or replacement of DNA at a specific site can be accomplished e.g. by homology-directed repair (HDR) or non-homologous end joining (NHEJ), or single base change editing. In some embodiments, a donor template is used, for example for HDR, such that a desired sequence within the donor template is inserted into the genome by a homologous recombination event. In one embodiment, a “donor template” or “repair template” comprises two homology arms (e.g., a 5′ homology arm and a 3′ homology arm) flanking on either side of a donor sequence comprising a desired mutation or insertion in the nucleic acid sequence to be introduced into the host genome. The 5′ and 3′ homology arms are substantially homologous to the genomic sequence of the target gene at the site of endonuclease mediated cutting. The 3′ homology arm is generally immediately downstream of the protospacer adjacent motif (PAM) site where the endonuclease cuts (e.g., a double stranded DNA cut), or in some embodiments, nicks the DNA.

As used herein, the term “gene editing system” refers to the minimum components necessary to effect genome editing in a cell. For example, a zinc finger nuclease or TALEN system may only require expression of the endonuclease fused to a nucleic acid complementary to the sequence of a target gene, whereas for a CRISPR/Cas gene editing system the minimum components may require e.g., a Cas endonuclease and a guide RNA. The gene editing system can be encoded on a single ceDNA vector or multiple vectors, as desired. Those of skill in the art will readily understand the component(s) necessary for a gene editing system.

As used herein, the term “base editing moiety” refers to an enzyme or enzyme system that can alter a single nucleotide in a sequence, for example, a cytosine/guanine nucleotide pair “G/C” to an adenine and thymine “T”/uridine “U” nucleotide pair (A/T, U) (see e.g., Shevidi et al. Dev Dyn 31 (2017) PMID:28857338; Kyoungmi et al. Nature Biotechnology 35:435-437 (2017), the contents of each of which are incorporated herein by reference in their entirety) or an adenine/thymine “A/T” nucleotide pair to a guanine/cytosine “G/C” nucleotide pair (see e.g., Gaudelli et al. Nature (2017), in press doi:10.1038/nature24644, the contents of which are incorporated herein by reference in its entirety).

As used herein, the term “genomic safe harbor gene” or “safe harbor gene” refers to a gene or locus that a nucleic acid sequence can be inserted such that the sequence can integrate and function in a predictable manner (e.g., express a protein of interest) without significant negative consequences to endogenous gene activity, or the promotion of cancer. In some embodiments, a safe harbor gene is also a locus or gene where an inserted nucleic acid sequence can be expressed efficiently and at higher levels than a non-safe harbor site.

As used herein, the term “gene delivery” means a process by which foreign DNA is transferred to host cells for applications of gene therapy.

As used herein, the term “CRISPR” stands for Clustered Regularly Interspaced Short Palindromic Repeats, which are the hallmark of a bacterial defense system that forms the basis for CRISPR-Cas9 genome editing technology.

As used herein, the term “zinc finger” means a small protein structural motif that is characterized by the coordination of one or more zinc ions, in order to stabilize the fold.

As used herein, the term “homologous recombination” means a type of genetic recombination in which nucleotide sequences are exchanged between two similar or identical molecules of DNA. Homologous recombination also produces new combinations of DNA sequences. These new combinations of DNA represent genetic variation. Homologous recombination is also used in horizontal gene transfer to exchange genetic material between different strains and species of viruses.

As used herein, the term “terminal repeat” or “TR” includes any viral terminal repeat or synthetic sequence that comprises at least one minimal required origin of replication and a region comprising a palindrome hairpin structure. A Rep-binding sequence (“RBS”) (also referred to as RBE (Rep-binding element)) and a terminal resolution site (“TRS”) together constitute a “minimal required origin of replication” and thus the TR comprises at least one RBS and at least one TRS. TRs that are the inverse complement of one another within a given stretch of polynucleotide sequence are typically each referred to as an “inverted terminal repeat” or “ITR”. In the context of a virus, ITRs mediate replication, virus packaging, integration and provirus rescue. As was unexpectedly found in the invention herein, TRs that are not inverse complements across their full length can still perform the traditional functions of ITRs, and thus the term ITR is used herein to refer to a TR in a ceDNA genome or ceDNA vector that is capable of mediating replication of ceDNA vector. It will be understood by one of ordinary skill in the art that in complex ceDNA vector configurations more than two ITRs or asymmetric ITR pairs may be present. The ITR can be an AAV ITR or a non-AAV ITR, or can be derived from an AAV ITR or a non-AAV ITR. For example, the ITR can be derived from the family Parvoviridae, which encompasses parvoviruses and dependoviruses (e.g., canine parvovirus, bovine parvovirus, mouse parvovirus, porcine parvovirus, human parvovirus B-19), or the SV40 hairpin that serves as the origin of SV40 replication can be used as an ITR, which can further be modified by truncation, substitution, deletion, insertion and/or addition. Parvoviridae family viruses consist of two subfamilies: Parvovirinae, which infect vertebrates, and Densovirinae, which infect invertebrates. Dependoparvoviruses include the viral family of the adeno-associated viruses (AAV) which are capable of replication in vertebrate hosts including, but not limited to, human, primate, bovine, canine, equine and ovine species. For convenience herein, an ITR located 5′ to (upstream of) an expression cassette in a ceDNA vector is referred to as a “5′ ITR” or a “left ITR”, and an ITR located 3′ to (downstream of) an expression cassette in a ceDNA vector is referred to as a “3′ ITR” or a “right ITR”.

As used herein, the term “substantially symmetrical WT-ITRs” or a “substantially symmetrical WT-ITR pair” refers to a pair of WT-ITRs within a single ceDNA genome or ceDNA vector that are both wild type ITRs that have an inverse complement sequence across their entire length. For example, an ITR can be considered to be a wild-type sequence, even if it has one or more nucleotides that deviate from the canonical naturally occurring sequence, so long as the changes do not affect the properties and overall three-dimensional structure of the sequence. In some aspects, the deviating nucleotides represent conservative sequence changes. As one non-limiting example, a sequence that has at least 95%, 96%, 97%, 98%, or 99% sequence identity to the canonical sequence (as measured, e.g., using BLAST at default settings), and also has a symmetrical three-dimensional spatial organization to the other WT-ITR such that their 3D structures are the same shape in geometrical space. The substantially symmetrical WT-ITR has the same A, C-C′ and B-B′ loops in 3D space. A substantially symmetrical WT-ITR can be functionally confirmed as WT by determining that it has an operable Rep binding site (RBE or RBE′) and terminal resolution site (trs) that pairs with the appropriate Rep protein. One can optionally test other functions, including transgene expression under permissive conditions.

As used herein, the phrases of “modified ITR” or “mod-ITR” or “mutant ITR” are used interchangeably herein and refer to an ITR that has a mutation in at least one or more nucleotides as compared to the WT-ITR from the same serotype. The mutation can result in a change in one or more of A, C, C′, B, B′ regions in the ITR, and can result in a change in the three-dimensional spatial organization (i.e. its 3D structure in geometric space) as compared to the 3D spatial organization of a WT-ITR of the same serotype.

As used herein, the term “asymmetric ITRs” also referred to herein as “asymmetric ITR pairs” refers to a pair of ITRs within a single ceDNA genome or ceDNA vector that are not inverse complements across their full length. The difference in sequence between the two ITRs may be due to nucleotide addition, deletion, truncation, or point mutation. In one embodiment, one ITR of the pair may be a wild-type AAV sequence and the other a non-wild-type or synthetic sequence. In another embodiment, neither ITR of the pair is a wild-type AAV sequence and the two ITRs differ in sequence from one another. For convenience herein, an ITR located 5′ to (upstream of) an expression cassette in a ceDNA vector is referred to as a “5′ ITR” or a “left ITR”, and an ITR located 3′ to (downstream of) an expression cassette in a ceDNA vector is referred to as a “3′ ITR” or a “right ITR”. As one non-limiting example, an asymmetric ITR pair does not have a symmetrical three-dimensional spatial organization to their cognate ITR such that their 3D structures are different shapes in geometrical space. Stated differently, an asymmetrical ITR pair have the different overall geometric structure, i.e., they have different organization of their A, C-C′ and B-B′ loops in 3D space (e.g., one ITR may have a short C-C′ arm and/or short B-B′ arm as compared to the cognate ITR). The difference in sequence between the two ITRs may be due to one or more nucleotide addition, deletion, truncation, or point mutation. In one embodiment, one ITR of the asymmetric ITR pair may be a wild-type AAV ITR sequence and the other ITR a modified ITR as defined herein (e.g., a non-wild-type or synthetic ITR sequence). In another embodiment, neither ITRs of the asymmetric ITR pair is a wild-type AAV sequence and the two ITRs are modified ITRs that have different shapes in geometrical space (i.e., a different overall geometric structure). In some embodiments, one mod-ITRs of an asymmetric ITR pair can have a short C-C′ arm and the other ITR can have a different modification (e.g., a single arm, or a short B-B′ arm etc.) such that they have different three-dimensional spatial organization as compared to the cognate asymmetric mod-ITR.

As used herein, the term “symmetric ITRs” refers to a pair of ITRs within a single ceDNA genome or ceDNA vector that are mutated or modified relative to wild-type dependoviral ITR sequences and are inverse complements across their full length. Neither ITRs are wild type ITR AAV2 sequences (i.e., they are a modified ITR, also referred to as a mutant ITR), and can have a difference in sequence from the wild type ITR due to nucleotide addition, deletion, substitution, truncation, or point mutation. For convenience herein, an ITR located 5′ to (upstream of) an expression cassette in a ceDNA vector is referred to as a “5′ ITR” or a “left ITR”, and an ITR located 3′ to (downstream of) an expression cassette in a ceDNA vector is referred to as a “3′ ITR” or a “right ITR”.

As used herein, the terms “substantially symmetrical modified-ITRs” or a “substantially symmetrical mod-ITR pair” refers to a pair of modified-ITRs within a single ceDNA genome or ceDNA vector that are both that have an inverse complement sequence across their entire length. For example, the a modified ITR can be considered substantially symmetrical, even if it has some nucleotide sequences that deviate from the inverse complement sequence so long as the changes do not affect the properties and overall shape. As one non-limiting example, a sequence that has at least 85%, 90%, 95%, 96%, 97%, 98%, or 99% sequence identity to the canonical sequence (as measured using BLAST at default settings), and also has a symmetrical three-dimensional spatial organization to their cognate modified ITR such that their 3D structures are the same shape in geometrical space. Stated differently, a substantially symmetrical modified-ITR pair have the same A, C-C′ and B-B′ loops organized in 3D space. In some embodiments, the ITRs from a mod-ITR pair may have different reverse complement nucleotide sequences but still have the same symmetrical three-dimensional spatial organization—that is both ITRs have mutations that result in the same overall 3D shape. For example, one ITR (e.g., 5′ ITR) in a mod-ITR pair can be from one serotype, and the other ITR (e.g., 3′ ITR) can be from a different serotype, however, both can have the same corresponding mutation (e.g., if the 5′ITR has a deletion in the C region, the cognate modified 3′ITR from a different serotype has a deletion at the corresponding position in the C′ region), such that the modified ITR pair has the same symmetrical three-dimensional spatial organization. In such embodiments, each ITR in a modified ITR pair can be from different serotypes (e.g. AAV1, 2, 3, 4, 5, 6, 7, 8, 9, 10, 11, and 12) such as the combination of AAV2 and AAV6, with the modification in one ITR reflected in the corresponding position in the cognate ITR from a different serotype. In one embodiment, a substantially symmetrical modified ITR pair refers to a pair of modified ITRs (mod-ITRs) so long as the difference in nucleotide sequences between the ITRs does not affect the properties or overall shape and they have substantially the same shape in 3D space. As a non-limiting example, a mod-ITR that has at least 95%, 96%, 97%, 98% or 99% sequence identity to the canonical mod-ITR as determined by standard means well known in the art such as BLAST (Basic Local Alignment Search Tool), or BLASTN at default settings, and also has a symmetrical three-dimensional spatial organization such that their 3D structure is the same shape in geometric space. A substantially symmetrical mod-ITR pair has the same A, C-C′ and B-B′ loops in 3D space, e.g., if a modified ITR in a substantially symmetrical mod-ITR pair has a deletion of a C-C′ arm, then the cognate mod-ITR has the corresponding deletion of the C-C′ loop and also has a similar 3D structure of the remaining A and B-B′ loops in the same shape in geometric space of its cognate mod-ITR.

The term “flanking” refers to a relative position of one nucleic acid sequence with respect to another nucleic acid sequence. Generally, in the sequence ABC, B is flanked by A and C. The same is true for the arrangement A×B×C. Thus, a flanking sequence precedes or follows a flanked sequence but need not be contiguous with, or immediately adjacent to the flanked sequence. In one embodiment, the term flanking refers to terminal repeats at each end of the linear duplex ceDNA vector.

As used herein, the term “ceDNA genome” refers to an expression cassette that further incorporates at least one inverted terminal repeat region. A ceDNA genome may further comprise one or more spacer regions. In some embodiments the ceDNA genome is incorporated as an intermolecular duplex polynucleotide of DNA into a plasmid or viral genome.

As used herein, the term “ceDNA spacer region” refers to an intervening sequence that separates functional elements in the ceDNA vector or ceDNA genome. In some embodiments, ceDNA spacer regions keep two functional elements at a desired distance for optimal functionality. In some embodiments, ceDNA spacer regions provide or add to the genetic stability of the ceDNA genome within e.g., a plasmid or baculovirus. In some embodiments, ceDNA spacer regions facilitate ready genetic manipulation of the ceDNA genome by providing a convenient location for cloning sites and the like. For example, in certain aspects, an oligonucleotide “polylinker” containing several restriction endonuclease sites, or a non-open reading frame sequence designed to have no known protein (e.g., transcription factor) binding sites can be positioned in the ceDNA genome to separate the cis-acting factors, e.g., inserting a 6mer, 12mer, 18mer, 24mer, 48mer, 86mer, 176mer, etc. between the terminal resolution site and the upstream transcriptional regulatory element. Similarly, the spacer may be incorporated between the polyadenylation signal sequence and the 3′-terminal resolution site.

As used herein, the terms “Rep binding site, “Rep binding element, “RBE” and “RBS” are used interchangeably and refer to a binding site for Rep protein (e.g., AAV Rep 78 or AAV Rep 68) which upon binding by a Rep protein permits the Rep protein to perform its site-specific endonuclease activity on the sequence incorporating the RBS. An RBS sequence and its inverse complement together form a single RBS. RBS sequences are known in the art, and include, for example, 5′-GCGCGCTCGCTCGCTC-3′ (SEQ ID NO: 60), an RBS sequence identified in AAV2. Any known RBS sequence may be used in the embodiments of the invention, including other known AAV RBS sequences and other naturally known or synthetic RBS sequences. Without being bound by theory it is thought that the nuclease domain of a Rep protein binds to the duplex nucleotide sequence GCTC, and thus the two known AAV Rep proteins bind directly to and stably assemble on the duplex oligonucleotide, 5′-(GCGC)(GCTC)(GCTC)(GCTC)-3′ (SEQ ID NO: 60). In addition, soluble aggregated conformers (i.e., undefined number of inter-associated Rep proteins) dissociate and bind to oligonucleotides that contain Rep binding sites. Each Rep protein interacts with both the nitrogenous bases and phosphodiester backbone on each strand. The interactions with the nitrogenous bases provide sequence specificity whereas the interactions with the phosphodiester backbone are non- or less-sequence specific and stabilize the protein-DNA complex.

As used herein, the terms “terminal resolution site” and “TRS” are used interchangeably herein and refer to a region at which Rep forms a tyrosine-phosphodiester bond with the 5′ thymidine generating a 3′ OH that serves as a substrate for DNA extension via a cellular DNA polymerase, e.g., DNA pol delta or DNA pol epsilon. Alternatively, the Rep-thymidine complex may participate in a coordinated ligation reaction. In some embodiments, a TRS minimally encompasses a non-base-paired thymidine. In some embodiments, the nicking efficiency of the TRS can be controlled at least in part by its distance within the same molecule from the RBS. When the acceptor substrate is the complementary ITR, then the resulting product is an intramolecular duplex. TRS sequences are known in the art, and include, for example, 5′-GGTTGA-3′ (SEQ ID NO: 61), the hexanucleotide sequence identified in AAV2. Any known TRS sequence may be used in the embodiments of the invention, including other known AAV TRS sequences and other naturally known or synthetic TRS sequences such as AGTT (SEQ ID NO: 62), GGTTGG (SEQ ID NO: 63), AGTTGG (SEQ ID NO: 64), AGTTGA (SEQ ID NO: 65), and other motifs such as RRTTRR (SEQ ID NO: 66).

As used herein, the term “ceDNA-plasmid” refers to a plasmid that comprises a ceDNA genome as an intermolecular duplex.

As used herein, the term “ceDNA-bacmid” refers to an infectious baculovirus genome comprising a ceDNA genome as an intermolecular duplex that is capable of propagating in E. coli as a plasmid, and so can operate as a shuttle vector for baculovirus.

As used herein, the term “ceDNA-baculovirus” refers to a baculovirus that comprises a ceDNA genome as an intermolecular duplex within the baculovirus genome.

As used herein, the terms “ceDNA-baculovirus infected insect cell” and “ceDNA-BIIC” are used interchangeably, and refer to an invertebrate host cell (including, but not limited to an insect cell (e.g., an Sf9 cell)) infected with a ceDNA-baculovirus.

As used herein, the term “closed-ended DNA vector” refers to a capsid-free DNA vector with at least one covalently closed end and where at least part of the vector has an intramolecular duplex structure.

As used herein, the terms “ceDNA vector” and “ceDNA” are used interchangeably and refer to a closed-ended DNA vector comprising at least one terminal palindrome. In some embodiments, the ceDNA comprises two covalently-closed ends.

As defined herein, “reporters” refer to proteins that can be used to provide detectable read-outs. Reporters generally produce a measurable signal such as fluorescence, color, or luminescence. Reporter protein coding sequences encode proteins whose presence in the cell or organism is readily observed. For example, fluorescent proteins cause a cell to fluoresce when excited with light of a particular wavelength, luciferases cause a cell to catalyze a reaction that produces light, and enzymes such as β-galactosidase convert a substrate to a colored product. Exemplary reporter polypeptides useful for experimental or diagnostic purposes include, but are not limited to β-lactamase, β-galactosidase (LacZ), alkaline phosphatase (AP), thymidine kinase (TK), green fluorescent protein (GFP) and other fluorescent proteins, chloramphenicol acetyltransferase (CAT), luciferase (e.g., SEQ ID NO: 56), and others well known in the art.

As used herein, the term “effector protein” refers to a polypeptide that provides a detectable read-out, either as, for example, a reporter polypeptide, or more appropriately, as a polypeptide that kills a cell, e.g., a toxin, or an agent that renders a cell susceptible to killing with a chosen agent or lack thereof. Effector proteins include any protein or peptide that directly targets or damages the host cell's DNA and/or RNA. For example, effector proteins can include, but are not limited to, a restriction endonuclease that targets a host cell DNA sequence (whether genomic or on an extrachromosomal element), a protease that degrades a polypeptide target necessary for cell survival, a DNA gyrase inhibitor, and a ribonuclease-type toxin. In some embodiments, the expression of an effector protein controlled by a synthetic biological circuit as described herein can participate as a factor in another synthetic biological circuit to thereby expand the range and complexity of a biological circuit system's responsiveness.

Transcriptional regulators refer to transcriptional activators and repressors that either activate or repress transcription of a gene of interest. Promoters are regions of nucleic acid that initiate transcription of a particular gene Transcriptional activators typically bind nearby to transcriptional promoters and recruit RNA polymerase to directly initiate transcription. Repressors bind to transcriptional promoters and sterically hinder transcriptional initiation by RNA polymerase. Other transcriptional regulators may serve as either an activator or a repressor depending on where they bind and cellular and environmental conditions. Non-limiting examples of transcriptional regulator classes include, but are not limited to homeodomain proteins, zinc-finger proteins, winged-helix (forkhead) proteins, and leucine-zipper proteins.

As used herein, a “repressor protein” or “inducer protein” is a protein that binds to a regulatory sequence element and represses or activates, respectively, the transcription of sequences operatively linked to the regulatory sequence element. Preferred repressor and inducer proteins as described herein are sensitive to the presence or absence of at least one input agent or environmental input. Preferred proteins as described herein are modular in form, comprising, for example, separable DNA-binding and input agent-binding or responsive elements or domains.

As used herein, “carrier” includes any and all solvents, dispersion media, vehicles, coatings, diluents, antibacterial and antifungal agents, isotonic and absorption delaying agents, buffers, carrier solutions, suspensions, colloids, and the like. The use of such media and agents for pharmaceutically active substances is well known in the art. Supplementary active ingredients can also be incorporated into the compositions. The phrase “pharmaceutically-acceptable” refers to molecular entities and compositions that do not produce a toxic, an allergic, or similar untoward reaction when administered to a host.

As used herein, an “input agent responsive domain” is a domain of a transcription factor that binds to or otherwise responds to a condition or input agent in a manner that renders a linked DNA binding fusion domain responsive to the presence of that condition or input. In one embodiment, the presence of the condition or input results in a conformational change in the input agent responsive domain, or in a protein to which it is fused, that modifies the transcription-modulating activity of the transcription factor.

The term “in vivo” refers to assays or processes that occur in or within an organism, such as a multicellular animal. In some of the aspects described herein, a method or use can be said to occur “in vivo” when a unicellular organism, such as a bacterium, is used. The term “ex vivo” refers to methods and uses that are performed using a living cell with an intact membrane that is outside of the body of a multicellular animal or plant, e.g., explants, cultured cells, including primary cells and cell lines, transformed cell lines, and extracted tissue or cells, including blood cells, among others. The term “in vitro” refers to assays and methods that do not require the presence of a cell with an intact membrane, such as cellular extracts, and can refer to the introducing of a programmable synthetic biological circuit in a non-cellular system, such as a medium not comprising cells or cellular systems, such as cellular extracts.

The term “promoter,” as used herein, refers to any nucleic acid sequence that regulates the expression of another nucleic acid sequence by driving transcription of the nucleic acid sequence, which can be a heterologous target gene encoding a protein or an RNA. Promoters can be constitutive, inducible, repressible, tissue-specific, or any combination thereof. A promoter is a control region of a nucleic acid sequence at which initiation and rate of transcription of the remainder of a nucleic acid sequence are controlled. A promoter can also contain genetic elements at which regulatory proteins and molecules can bind, such as RNA polymerase and other transcription factors. In some embodiments of the aspects described herein, a promoter can drive the expression of a transcription factor that regulates the expression of the promoter itself. Within the promoter sequence will be found a transcription initiation site, as well as protein binding domains responsible for the binding of RNA polymerase. Eukaryotic promoters will often, but not always, contain “TATA” boxes and “CAT” boxes. Various promoters, including inducible promoters, may be used to drive the expression of transgenes in the ceDNA vectors disclosed herein. A promoter sequence may be bounded at its 3′ terminus by the transcription initiation site and extends upstream (5′ direction) to include the minimum number of bases or elements necessary to initiate transcription at levels detectable above background.

The term “enhancer” as used herein refers to a cis-acting regulatory sequence (e.g., 50-1,500 base pairs) that binds one or more proteins (e.g., activator proteins, or transcription factor) to increase transcriptional activation of a nucleic acid sequence Enhancers can be positioned up to 1,000,000 base pars upstream of the gene start site or downstream of the gene start site that they regulate. An enhancer can be positioned within an intronic region, or in the exonic region of an unrelated gene.

A promoter can be said to drive expression or drive transcription of the nucleic acid sequence that it regulates. The phrases “operably linked,” “operatively positioned,” “operatively linked,” “under control,” and “under transcriptional control” indicate that a promoter is in a correct functional location and/or orientation in relation to a nucleic acid sequence it regulates to control transcriptional initiation and/or expression of that sequence. An “inverted promoter,” as used herein, refers to a promoter in which the nucleic acid sequence is in the reverse orientation, such that what was the coding strand is now the non-coding strand, and vice versa. Inverted promoter sequences can be used in various embodiments to regulate the state of a switch. In addition, in various embodiments, a promoter can be used in conjunction with an enhancer.

A promoter can be one naturally associated with a gene or sequence, as can be obtained by isolating the 5′ non-coding sequences located upstream of the coding segment and/or exon of a given gene or sequence. Such a promoter can be referred to as “endogenous.” Similarly, in some embodiments, an enhancer can be one naturally associated with a nucleic acid sequence, located either downstream or upstream of that sequence.

In some embodiments, a coding nucleic acid segment is positioned under the control of a “recombinant promoter” or “heterologous promoter,” both of which refer to a promoter that is not normally associated with the encoded nucleic acid sequence it is operably linked to in its natural environment. A recombinant or heterologous enhancer refers to an enhancer not normally associated with a given nucleic acid sequence in its natural environment. Such promoters or enhancers can include promoters or enhancers of other genes; promoters or enhancers isolated from any other prokaryotic, viral, or eukaryotic cell; and synthetic promoters or enhancers that are not “naturally occurring,” i.e., comprise different elements of different transcriptional regulatory regions, and/or mutations that alter expression through methods of genetic engineering that are known in the art. In addition to producing nucleic acid sequences of promoters and enhancers synthetically, promoter sequences can be produced using recombinant cloning and/or nucleic acid amplification technology, including PCR, in connection with the synthetic biological circuits and modules disclosed herein (see, e.g., U.S. Pat. Nos. 4,683,202, 5,928,906, each incorporated herein by reference). Furthermore, it is contemplated that control sequences that direct transcription and/or expression of sequences within non-nuclear organelles such as mitochondria, chloroplasts, and the like, can be employed as well.

As described herein, an “inducible promoter” is one that is characterized by initiating or enhancing transcriptional activity when in the presence of, influenced by, or contacted by an inducer or inducing agent. An “inducer” or “inducing agent,” as defined herein, can be endogenous, or a normally exogenous compound or protein that is administered in such a way as to be active in inducing transcriptional activity from the inducible promoter. In some embodiments, the inducer or inducing agent, i.e., a chemical, a compound or a protein, can itself be the result of transcription or expression of a nucleic acid sequence (i.e., an inducer can be an inducer protein expressed by another component or module), which itself can be under the control or an inducible promoter. In some embodiments, an inducible promoter is induced in the absence of certain agents, such as a repressor. Examples of inducible promoters include but are not limited to, tetracycline, metallothionine, ecdysone, mammalian viruses (e.g., the adenovirus late promoter; and the mouse mammary tumor virus long terminal repeat (MMTV-LTR)) and other steroid-responsive promoters, rapamycin responsive promoters and the like.

The terms “DNA regulatory sequences,” “control elements,” and “regulatory elements,” used interchangeably herein, refer to transcriptional and translational control sequences, such as promoters, enhancers, polyadenylation signals, terminators, protein degradation signals, and the like, that provide for and/or regulate transcription of a non-coding sequence (e.g., DNA-targeting RNA) or a coding sequence (e.g., site-directed modifying polypeptide, or Cas9/Csn1 polypeptide) and/or regulate translation of an encoded polypeptide.

The term “operably linked” refers to a juxtaposition wherein the components so described are in a relationship permitting them to function in their intended manner. For instance, a promoter is operably linked to a coding sequence if the promoter affects its transcription or expression. An “expression cassette” includes an exogenous DNA sequence that is operably linked to a promoter or other regulatory sequence sufficient to direct transcription of the transgene in the ceDNA vector. Suitable promoters include, for example, tissue specific promoters. Promoters can also be of AAV origin.

The term “subject” as used herein refers to a human or animal, to whom treatment, including prophylactic treatment, with the ceDNA vector according to the present invention, is provided. Usually the animal is a vertebrate such as, but not limited to a primate, rodent, domestic animal or game animal. Primates include but are not limited to, chimpanzees, cynomologous monkeys, spider monkeys, and macaques, e.g., Rhesus. Rodents include mice, rats, woodchucks, ferrets, rabbits and hamsters. Domestic and game animals include, but are not limited to, cows, horses, pigs, deer, bison, buffalo, feline species, e.g., domestic cat, canine species, e.g., dog, fox, wolf, avian species, e.g., chicken, emu, ostrich, and fish, e.g., trout, catfish and salmon. In certain embodiments of the aspects described herein, the subject is a mammal, e.g., a primate or a human. A subject can be male or female. Additionally, a subject can be an infant or a child. In some embodiments, the subject can be a neonate or an unborn subject, e.g., the subject is in utero. Preferably, the subject is a mammal. The mammal can be a human, non-human primate, mouse, rat, dog, cat, horse, or cow, but is not limited to these examples. Mammals other than humans can be advantageously used as subjects that represent animal models of diseases and disorders. In addition, the methods and compositions described herein can be used for domesticated animals and/or pets. A human subject can be of any age, gender, race or ethnic group, e.g., Caucasian (white), Asian, African, black, African American, African European, Hispanic, Mideastern, etc. In some embodiments, the subject can be a patient or other subject in a clinical setting. In some embodiments, the subject is already undergoing treatment. In some embodiments, the subject is an embryo, a fetus, neonate, infant, child, adolescent, or adult. In some embodiments, the subject is a human fetus, human neonate, human infant, human child, human adolescent, or human adult. In some embodiments, the subject is an animal embryo, or non-human embryo or non-human primate embryo. In some embodiments, the subject is a human embryo.

As used herein, the term “host cell”, includes any cell type that is susceptible to transformation, transfection, transduction, and the like with a nucleic acid construct or ceDNA expression vector of the present disclosure. As non-limiting examples, a host cell can be an isolated primary cell, pluripotent stem cells, CD34⁺ cells), induced pluripotent stem cells, or any of a number of immortalized cell lines (e.g., HepG2 cells). Alternatively, a host cell can be an in situ or in vivo cell in a tissue, organ or organism.

The term “exogenous” refers to a substance present in a cell other than its native source. The term “exogenous” when used herein can refer to a nucleic acid (e.g., a nucleic acid encoding a polypeptide) or a polypeptide that has been introduced by a process involving the hand of man into a biological system such as a cell or organism in which it is not normally found and one wishes to introduce the nucleic acid or polypeptide into such a cell or organism. Alternatively, “exogenous” can refer to a nucleic acid or a polypeptide that has been introduced by a process involving the hand of man into a biological system such as a cell or organism in which it is found in relatively low amounts and one wishes to increase the amount of the nucleic acid or polypeptide in the cell or organism, e.g., to create ectopic expression or levels. In contrast, the term “endogenous” refers to a substance that is native to the biological system or cell.

The term “sequence identity” refers to the relatedness between two nucleotide sequences. For purposes of the present disclosure, the degree of sequence identity between two deoxyribonucleotide sequences is determined using the Needleman-Wunsch algorithm (Needleman and Wunsch, 1970, supra) as implemented in the Needle program of the EMBOSS package (EMBOSS: The European Molecular Biology Open Software Suite, Rice et al., 2000, supra), preferably version 3.0.0 or later. The optional parameters used are gap open penalty of 10, gap extension penalty of 0.5, and the EDNAFULL (EMBOSS version of NCBI NUC4.4) substitution matrix. The output of Needle labeled “longest identity” (obtained using the—nobrief option) is used as the percent identity and is calculated as follows: (Identical Deoxyribonucleotides.times.100)/(Length of Alignment-Total Number of Gaps in Alignment). The length of the alignment is preferably at least 10 nucleotides, preferably at least 25 nucleotides more preferred at least 50 nucleotides and most preferred at least 100 nucleotides.

The term “homology” or “homologous” as used herein is defined as the percentage of nucleotide residues in the homology arm that are identical to the nucleotide residues in the corresponding sequence on the target chromosome, after aligning the sequences and introducing gaps, if necessary, to achieve the maximum percent sequence identity. Alignment for purposes of determining percent nucleotide sequence homology can be achieved in various ways that are within the skill in the art, for instance, using publicly available computer software such as BLAST, BLAST-2, ALIGN, ClustalW2 or Megalign (DNASTAR) software. Those skilled in the art can determine appropriate parameters for aligning sequences, including any algorithms needed to achieve maximal alignment over the full length of the sequences being compared. In some embodiments, a nucleic acid sequence (e.g., DNA sequence), for example of a homology arm of a repair template, is considered “homologous” when the sequence is at least 70%, at least 75%, at least 80%, at least 85%, at least 90%, at least 91%, at least 92%, at least 93%, at least 94%, at least 95%, at least 96%, at least 97%, at least 98%, at least 99%, or more, identical to the corresponding native or unedited nucleic acid sequence (e.g., genomic sequence) of the host cell.

As used herein, a “homology arm” refers to a polynucleotide that is suitable to target a donor sequence to a genome through homologous recombination. Typically, two homology arms flank the donor sequence, wherein each homology arm comprises genomic sequences upstream and downstream of the locus of integration.

As used herein, “a donor sequence” refers to a polynucleotide that is to be inserted into, or used as a repair template for, a host cell genome. The donor sequence can comprise the modification which is desired to be made during gene editing. The sequence to be incorporated can be introduced into the target nucleic acid molecule via homology directed repair at the target sequence, thereby causing an alteration of the target sequence from the original target sequence to the sequence comprised by the donor sequence. Accordingly, the sequence comprised by the donor sequence can be, relative to the target sequence, an insertion, a deletion, an indel, a point mutation, a repair of a mutation, etc. The donor sequence can be, e.g., a single-stranded DNA molecule; a double-stranded DNA molecule; a DNA/RNA hybrid molecule; and a DNA/modRNA (modified RNA) hybrid molecule. In one embodiment, the donor sequence is foreign to the homology arms. The editing can be RNA as well as DNA editing. The donor sequence can be endogenous to or exogenous to the host cell genome, depending upon the nature of the desired gene editing.

The term “heterologous,” as used herein, means a nucleotide or polypeptide sequence that is not found in the native nucleic acid or protein, respectively. For example, in a chimeric Cas9/Csn1 protein, the RNA-binding domain of a naturally-occurring bacterial Cas9/Csn1 polypeptide (or a variant thereof) may be fused to a heterologous polypeptide sequence (i.e. a polypeptide sequence from a protein other than Cas9/Csn1 or a polypeptide sequence from another organism). The heterologous polypeptide sequence may exhibit an activity (e.g., enzymatic activity) that will also be exhibited by the chimeric Cas9/Csn1 protein (e.g., methyltransferase activity, acetyltransferase activity, kinase activity, ubiquitinating activity, etc.). A heterologous nucleic acid sequence may be linked to a naturally-occurring nucleic acid sequence (or a variant thereof) (e.g., by genetic engineering) to generate a chimeric nucleotide sequence encoding a chimeric polypeptide. As another example, in a fusion variant Cas9 site-directed polypeptide, a variant Cas9 site-directed polypeptide may be fused to a heterologous polypeptide (i.e. a polypeptide other than Cas9), which exhibits an activity that will also be exhibited by the fusion variant Cas9 site-directed polypeptide. A heterologous nucleic acid sequence may be linked to a variant Cas9 site-directed polypeptide (e.g., by genetic engineering) to generate a nucleotide sequence encoding a fusion variant Cas9 site-directed polypeptide.

A “vector” or “expression vector” is a replicon, such as plasmid, bacmid, phage, virus, virion, or cosmid, to which another DNA segment, i.e. an “insert”, may be attached so as to bring about the replication of the attached segment in a cell. A vector can be a nucleic acid construct designed for delivery to a host cell or for transfer between different host cells. As used herein, a vector can be viral or non-viral in origin and/or in final form, however for the purpose of the present disclosure, a “vector” generally refers to a ceDNA vector, as that term is used herein. The term “vector” encompasses any genetic element that is capable of replication when associated with the proper control elements and that can transfer gene sequences to cells. In some embodiments, a vector can be an expression vector or recombinant vector.

As used herein, the term “expression vector” refers to a vector that directs expression of an RNA or polypeptide from sequences linked to transcriptional regulatory sequences on the vector. The sequences expressed will often, but not necessarily, be heterologous to the cell. An expression vector may comprise additional elements, for example, the expression vector may have two replication systems, thus allowing it to be maintained in two organisms, for example in human cells for expression and in a prokaryotic host for cloning and amplification. The term “expression” refers to the cellular processes involved in producing RNA and proteins and as appropriate, secreting proteins, including where applicable, but not limited to, for example, transcription, transcript processing, translation and protein folding, modification and processing. “Expression products” include RNA transcribed from a gene, and polypeptides obtained by translation of mRNA transcribed from a gene. The term “gene” means the nucleic acid sequence which is transcribed (DNA) to RNA in vitro or in vivo when operably linked to appropriate regulatory sequences. The gene may or may not include regions preceding and following the coding region, e.g., 5′ untranslated (5′UTR) or “leader” sequences and 3′ UTR or “trailer” sequences, as well as intervening sequences (introns) between individual coding segments (exons).

By “recombinant vector” is meant a vector that includes a heterologous nucleic acid sequence, or “transgene” that is capable of expression in vivo. It should be understood that the vectors described herein can, in some embodiments, be combined with other suitable compositions and therapies. In some embodiments, the vector is episomal. The use of a suitable episomal vector provides a means of maintaining the nucleotide of interest in the subject in high copy number extra chromosomal DNA thereby eliminating potential effects of chromosomal integration.

The terms “correcting”, “genome editing” and “restoring” as used herein refers to changing a mutant gene that encodes a truncated protein or no protein at all, such that a full-length functional or partially full-length functional protein expression is obtained. Correcting or restoring a mutant gene may include replacing the region of the gene that has the mutation or replacing the entire mutant gene with a copy of the gene that does not have the mutation with a repair mechanism such as homology-directed repair (HDR). Correcting or restoring a mutant gene may also include repairing a frameshift mutation that causes a premature stop codon, an aberrant splice acceptor site or an aberrant splice donor site, by generating a double stranded break in the gene that is then repaired using non-homologous end joining (NHEJ). NHEJ may add or delete at least one base pair during repair which may restore the proper reading frame and eliminate the premature stop codon. Correcting or restoring a mutant gene may also include disrupting an aberrant splice acceptor site or splice donor sequence. Correcting or restoring a mutant gene may also include deleting a non-essential gene segment by the simultaneous action of two nucleases on the same DNA strand in order to restore the proper reading frame by removing the DNA between the two nuclease target sites and repairing the DNA break by NHEJ.

The phrase “genetic disease” as used herein refers to a disease, partially or completely, directly or indirectly, caused by one or more abnormalities in the genome, especially a condition that is present from birth. The abnormality may be a mutation, an insertion or a deletion. The abnormality may affect the coding sequence of the gene or its regulatory sequence. The genetic disease may be, but not limited to DMD, hemophilia, cystic fibrosis, Huntington's chorea, familial hypercholesterolemia (LDL receptor defect), hepatoblastoma, Wilson's disease, congenital hepatic porphyria, inherited disorders of hepatic metabolism, Lesch Nyhan syndrome, sickle cell anemia, thalassaemias, xeroderma pigmentosum, Fanconi's anemia, retinitis pigmentosa, ataxia telangiectasia, Bloom's syndrome, retinoblastoma, and Tay-Sachs disease.

The phrase “non-homologous end joining (NHEJ) pathway” as used herein refers to a pathway that repairs double-strand breaks in DNA by directly ligating the break ends without the need for a homologous template. The template-independent re-ligation of DNA ends by NHEJ is a stochastic, error-prone repair process that introduces random micro-insertions and micro-deletions (indels) at the DNA breakpoint. This method may be used to intentionally disrupt, delete, or alter the reading frame of targeted gene sequences. NHEJ typically uses short homologous DNA sequences called microhomologies to guide repair. These microhomologies are often present in single-stranded overhangs on the end of double-strand breaks. When the overhangs are perfectly compatible, NHEJ usually repairs the break accurately, yet imprecise repair leading to loss of nucleotides may also occur, but is much more common when the overhangs are not compatible “Nuclease mediated NHEJ” as used herein refers to NHEJ that is initiated after a nuclease, such as a cas9 or other nuclease, cuts double stranded DNA. In a CRISPR/CAS system NHEJ can be targeted by using a single guide RNA sequence.

The phrase “homology-directed repair” or “HDR” as used interchangeably herein refers to a mechanism in cells to repair double strand DNA lesions when a homologous piece of DNA is present in the nucleus. HDR uses a donor DNA template to guide repair and may be used to create specific sequence changes to the genome, including the targeted addition of whole genes. If a donor template is provided along with the site specific nuclease, such as with a CRISPR/Cas9-based systems, then the cellular machinery will repair the break by homologous recombination, which is enhanced several orders of magnitude in the presence of DNA cleavage. When the homologous DNA piece is absent, non-homologous end joining may take place instead. In a CRISPR/Cas system one guide RNA, or two different guide RNAS can be used for HDR.

The phrase “repeat variable diresidue” or “RVD” as used interchangeably herein refers to a pair of adjacent amino acid residues within a DNA recognition motif (also known as “RVD module”), which includes 33-35 amino acids, of a TALE DNA-binding domain. The RVD determines the nucleotide specificity of the RVD module. RVD modules may be combined to produce an RVD array. The “RVD array length” as used herein refers to the number of RVD modules that corresponds to the length of the nucleotide sequence within the TALEN target region that is recognized by a TALEN, i.e., the binding region.

The terms “site-specific nuclease” or “sequence specific nuclease” as used herein refers to an enzyme capable of specifically recognizing and cleaving DNA sequences. The site-specific nuclease may be engineered. Examples of engineered site-specific nucleases include zinc finger nucleases (ZFNs), TAL effector nucleases (TALENs), and CRISPR/Cas-based systems, that use various natural and unnatural Cas enzymes.

As used herein the term “comprising” or “comprises” is used in reference to compositions, methods, and respective component(s) thereof, that are essential to the method or composition, yet open to the inclusion of unspecified elements, whether essential or not.

As used herein the term “consisting essentially of” refers to those elements required for a given embodiment. The term permits the presence of elements that do not materially affect the basic and novel or functional characteristic(s) of that embodiment. The use of “comprising” indicates inclusion rather than limitation.

The term “consisting of” refers to compositions, methods, and respective components thereof as described herein, which are exclusive of any element not recited in that description of the embodiment.

As used herein the term “consisting essentially of” refers to those elements required for a given embodiment. The term permits the presence of additional elements that do not materially affect the basic and novel or functional characteristic(s) of that embodiment of the invention.

As used in this specification and the appended claims, the singular forms “a,” “an,” and “the” include plural references unless the context clearly dictates otherwise. Thus for example, references to “the method” includes one or more methods, and/or steps of the type described herein and/or which will become apparent to those persons skilled in the art upon reading this disclosure and so forth. Similarly, the word “or” is intended to include “and” unless the context clearly indicates otherwise. Although methods and materials similar or equivalent to those described herein can be used in the practice or testing of this disclosure, suitable methods and materials are described below. The abbreviation, “e.g.” is derived from the Latin exempli gratia, and is used herein to indicate a non-limiting example. Thus, the abbreviation “e.g.” is synonymous with the term “for example.”

Other than in the operating examples, or where otherwise indicated, all numbers expressing quantities of ingredients or reaction conditions used herein should be understood as modified in all instances by the term “about.” The term “about” when used in connection with percentages can mean±1%. The present invention is further explained in detail by the following examples, but the scope of the invention should not be limited thereto.

Groupings of alternative elements or embodiments of the invention disclosed herein are not to be construed as limitations. Each group member can be referred to and claimed individually or in any combination with other members of the group or other elements found herein. One or more members of a group can be included in, or deleted from, a group for reasons of convenience and/or patentability. When any such inclusion or deletion occurs, the specification is herein deemed to contain the group as modified thus fulfilling the written description of all Markush groups used in the appended claims.

In some embodiments of any of the aspects, the disclosure described herein does not concern a process for cloning human beings, processes for modifying the germ line genetic identity of human beings, uses of human embryos for industrial or commercial purposes or processes for modifying the genetic identity of animals which are likely to cause them suffering without any substantial medical benefit to man or animal, and also animals resulting from such processes.

Other terms are defined herein within the description of the various aspects of the invention.

All patents and other publications; including literature references, issued patents, published patent applications, and co-pending patent applications; cited throughout this application are expressly incorporated herein by reference for the purpose of describing and disclosing, for example, the methodologies described in such publications that might be used in connection with the technology described herein. These publications are provided solely for their disclosure prior to the filing date of the present application. Nothing in this regard should be construed as an admission that the inventors are not entitled to antedate such disclosure by virtue of prior invention or for any other reason. All statements as to the date or representation as to the contents of these documents is based on the information available to the applicants and does not constitute any admission as to the correctness of the dates or contents of these documents.

The description of embodiments of the disclosure is not intended to be exhaustive or to limit the disclosure to the precise form disclosed. While specific embodiments of, and examples for, the disclosure are described herein for illustrative purposes, various equivalent modifications are possible within the scope of the disclosure, as those skilled in the relevant art will recognize. For example, while method steps or functions are presented in a given order, alternative embodiments may perform functions in a different order, or functions may be performed substantially concurrently. The teachings of the disclosure provided herein can be applied to other procedures or methods as appropriate. The various embodiments described herein can be combined to provide further embodiments. Aspects of the disclosure can be modified, if necessary, to employ the compositions, functions and concepts of the above references and application to provide yet further embodiments of the disclosure. Moreover, due to biological functional equivalency considerations, some changes can be made in protein structure without affecting the biological or chemical action in kind or amount. These and other changes can be made to the disclosure in light of the detailed description. All such modifications are intended to be included within the scope of the appended claims.

Specific elements of any of the foregoing embodiments can be combined or substituted for elements in other embodiments. Furthermore, while advantages associated with certain embodiments of the disclosure have been described in the context of these embodiments, other embodiments may also exhibit such advantages, and not all embodiments need necessarily exhibit such advantages to fall within the scope of the disclosure.

The technology described herein is further illustrated by the following examples which in no way should be construed as being further limiting.

It should be understood that this invention is not limited to the particular methodology, protocols, and reagents, etc., described herein and as such can vary. The terminology used herein is for the purpose of describing particular embodiments only, and is not intended to limit the scope of the present invention, which is defined solely by the claims.

By “nucleic acid of interest” is meant any nucleic acid sequence (including DNA and RNA sequences) which encodes a protein, RNA or other molecule which is desirable for delivery to a mammalian host cell. The sequence is generally operatively linked to other sequences which are needed for its expression such as a promoter. The phrase “nucleic acid of interest” is not meant to be limiting to DNA, but includes any nucleic acid (e.g., RNA or DNA) that encodes a protein or other molecule desirable for administration.

The term “nucleic acid construct” as used herein refers to a nucleic acid molecule, either single- or double-stranded, which is isolated from a naturally occurring gene or which is modified to con-tain segments of nucleic acids in a manner that would not otherwise exist in nature or which is synthetic. The term nucleic acid construct is synonymous with the term “expression cassette” when the nucleic acid construct contains the control sequences required for expression of a coding sequence of the present disclosure. An “expression cassette” includes a DNA coding sequence operably linked to a promoter.

By “hybridizable” or “complementary” or “substantially complementary” it is meant that a nucleic acid (e.g., RNA) includes a sequence of nucleotides that enables it to non-covalently bind, i.e. form Watson-Crick base pairs and/or G/U base pairs, “anneal”, or “hybridize,” to another nucleic acid in a sequence-specific, antiparallel, manner (i.e., a nucleic acid specifically binds to a complementary nucleic acid) under the appropriate in vitro and/or in vivo conditions of temperature and solution ionic strength. As is known in the art, standard Watson-Crick base-pairing includes: adenine (A) pairing with thymidine (T), adenine (A) pairing with uracil (U), and guanine (G) pairing with cytosine (C) [DNA, RNA]. In addition, it is also known in the art that for hybridization between two RNA molecules (e.g., dsRNA), guanine (G) base pairs with uracil (U). For example, G/U base-pairing is partially responsible for the degeneracy (i.e., redundancy) of the genetic code in the con-text of tRNA anti-codon base-pairing with codons in mRNA. In the context of this disclosure, a guanine (G) of a protein-binding segment (dsRNA duplex) of a subject DNA-targeting RNA mole-cule is considered complementary to a uracil (U), and vice versa. As such, when a G/U base-pair can be made at a given nucleotide position a protein-binding segment (dsRNA duplex) of a subject DNA-targeting RNA molecule, the position is not considered to be non-complementary, but is in-stead considered to be complementary.

The terms “peptide,” “polypeptide,” and “protein” are used interchangeably herein, and refer to a polymeric form of amino acids of any length, which can include coded and non-coded amino ac-ids, chemically or biochemically modified or derivatized amino acids, and polypeptides having modified peptide backbones.

A DNA sequence that “encodes” a particular RNA or protein gene product is a DNA nucleic acid sequence that is transcribed into the particular RNA and/or protein. A DNA polynucleotide may encode an RNA (mRNA) that is translated into protein, or a DNA polynucleotide may encode an RNA that is not translated into protein (e.g., tRNA, rRNA, or a DNA-targeting RNA; also called “non-coding” RNA or “ncRNA”).

As used herein, a “promoter sequence” is a DNA regulatory region capable of binding RNA polymerase and initiating transcription of a downstream (3′ direction) coding or non-coding sequence. A promoter sequence may be bounded at its 3′ terminus by the transcription initiation site and ex-tends upstream (5′ direction) to include the minimum number of bases or elements necessary to initiate transcription at levels detectable above background. Within the promoter sequence will be found a transcription initiation site, as well as protein binding domains responsible for the binding of RNA polymerase. Eukaryotic promoters will often, but not always, contain “TATA” boxes and “CAT” boxes. Various promoters, including inducible promoters, may be used to drive the various ceDNA vectors of the present disclosure.

The terms “DNA regulatory sequences,” “control elements,” and “regulatory elements,” used inter-changeably herein, refer to transcriptional and translational control sequences, such as promoters, enhancers, polyadenylation signals, terminators, protein degradation signals, and the like, that pro-vide for and/or regulate transcription of a non-coding sequence (e.g., DNA-targeting RNA) or a coding sequence (e.g., site-directed modifying polypeptide, or Cas9/Csn1 polypeptide) and/or regulate translation of an encoded polypeptide. Typical “control elements” include, but are not limited to transcription promoters, transcription enhancer elements, cis-acting transcription regulating elements (transcription regulators, a cis-acting element that affects the transcription of a gene, for example, a region of a promoter with which a transcription factor interacts to modulate expression of a gene), transcription termination signals, as well as polyadenylation sequences (located 5′ to the translation stop codon), sequences for optimization of initiation of translation (located 5′ to the coding sequence), translation enhancing sequences, and translation termination sequences. Control elements are derived from any include functional fragments thereof, for example, polynucleotides between about 5 and about 50 nucleotides in length (or any integer there between); preferably between about 5 and about 25 nucleotides (or any integer there between), even more preferably between about 5 and about 10 nucleotides (or any integer there between), and most preferably 9-10 nucleotides. Transcription promoters can include inducible promoters (where expression of a polynucleotide sequence operably linked to the promoter is induced by an analyte, cofactor, regulatory protein, etc.), repressible promoters (where expression of a polynucleotide sequence operably linked to the promoter is repressed by an analyte, cofactor, regulatory protein, etc.), and constitutive promoters.

The terms “operative linkage” and “operatively linked” (or “operably linked”) are used interchangeably with reference to a juxtaposition of two or more components (such as sequence elements), in which the components are arranged such that both components function normally and allow the possibility that at least one of the components can mediate a function that is exerted upon at least one of the other components. By way of illustration, a transcriptional regulatory sequence, such as a promoter, is operatively linked to a coding sequence if the promoter controls the level of transcription of the coding sequence in response to the presence or absence of one or more transcriptional regulatory factors on the promoter sequence. A transcriptional regulatory sequence is generally operatively linked in cis with a coding sequence, but need not be directly adjacent to it. For example, an enhancer is a transcriptional regulatory sequence that is operatively linked to a coding sequence, even though they are not contiguous.

An “expression cassette” includes an exogenous DNA sequence that is operably linked to a promoter or other regulatory sequence sufficient to direct transcription of the transgene in the ceDNA vector. Suitable promoters include, for example, tissue specific promoters. Promoters can also be of AAV origin. An expression cassette in a ceDNA vector described herein can include, for example, an expressible exogenous sequence (e.g., open reading frame) that encodes a protein that is either absent, inactive, or insufficient activity in the recipient subject or a gene that encodes a protein having a desired biological or a therapeutic effect. The exogenous sequence such as a donor sequence can encode a gene product that can function to correct the expression of a defective gene or transcript. The expression cassette can also encode corrective DNA strands, encode polypeptides, sense or antisense oligonucleotides, or RNAs (coding or non-coding; e.g., siRNAs, shRNAs, micro-RNAs, and their antisense counterparts (e.g., antagoMiR)). Expression cassettes can include an exogenous sequence that encodes a marker protein (also referred to as a reporter protein) to be used for experimental or diagnostic purposes, such as β-lactamase, β-galactosidase (LacZ), alkaline phosphatase, thymidine kinase, green fluorescent protein (GFP), chloramphenicol acetyltransferase (CAT), luciferase (e.g., SEQ ID NO: 56), and others well known in the art. A “marker gene” or “reporter gene” or “reporter sequence” are used interchangeably herein, and refers to any sequence that produces a protein product that is easily measured, preferably in a routine assay. Suitable marker genes include, but are not limited to, Mel1, chloramphenicol acetyl transferase (CAT), light generating proteins such as GFP, luciferase and/or β-galactosidase. Suitable marker genes may also encode markers or enzymes that can be measured in vivo such as thymidine kinase, measured in vivo using PET scanning, or luciferase, measured in vivo via whole body luminometric imaging. Selectable markers can also be used instead of, or in addition to, reporters. Positive selection markers are those polynucleotides that encode a product that enables only cells that carry and express the gene to survive and/or grow under certain conditions. For example, cells that express neomycin resistance (Ned) gene are resistant to the compound G418, while cells that do not express Ned are skilled by G418. Other examples of positive selection markers including hygromycin resistance and the like will be known to those of skill in the art. Negative selection markers are those polynucleotides that encode a product that enables only cells that carry and express the gene to be killed under certain conditions. For example, cells that express thymidine kinase (e.g., herpes simplex virus thymidine kinase, HSV-TK) are killed when gancyclovir is added. Other negative selection markers are known to those skilled in the art. The selectable marker need not be a transgene and, additionally, reporters and selectable markers can be used in various combinations.

In principle, the expression cassette can include any gene that encodes a protein, polypeptide or RNA that is either reduced or absent due to a mutation or which conveys a therapeutic benefit when overexpressed is considered to be within the scope of the disclosure. The ceDNA vector may comprise a template or donor nucleotide sequence used as a correcting DNA strand to be inserted after a double-strand break (or nick) provided by a nuclease. The ceDNA vector may include a template nucleotide sequence used as a correcting DNA strand to be inserted after a double-strand break (or nick) provided by a guided RNA nuclease, meganuclease, or zinc finger nuclease. Preferably, non-inserted bacterial DNA is not present and preferably no bacterial DNA is present in the ceDNA vectors provided herein. In some instances, the protein can change a codon without a nick.

Sequences provided in the expression cassette, expression construct, or donor sequence of a ceDNA vector described herein can be codon optimized for the host cell. As used herein, the term “codon optimized” or “codon optimization” refers to the process of modifying a nucleic acid sequence for enhanced expression in the cells of the vertebrate of interest, e.g., mouse or human, by replacing at least one, more than one, or a significant number of codons of the native sequence (e.g., a prokaryotic sequence) with codons that are more frequently or most frequently used in the genes of that vertebrate. Various species exhibit particular bias for certain codons of a particular amino acid. Typically, codon optimization does not alter the amino acid sequence of the original translated protein. Optimized codons can be determined using e.g., Aptagen's Gene Forge® codon optimization and custom gene synthesis platform (Aptagen, Inc., 2190 Fox Mill Rd. Suite 300, Herndon, Va. 20171) or another publicly available database.

Many organisms display a bias for use of particular codons to code for insertion of a particular amino acid in a growing peptide chain. Codon preference or codon bias, differences in codon us-age between organisms, is afforded by degeneracy of the genetic code, and is well documented among many organisms. Codon bias often correlates with the efficiency of translation of messenger RNA (mRNA), which is in turn believed to be dependent on, inter alia, the properties of the codons being translated and the availability of particular transfer RNA (tRNA) molecules. The predominance of selected tRNAs in a cell is generally a reflection of the codons used most frequently in peptide synthesis. Accordingly, genes can be tailored for optimal gene expression in a given organ-ism based on codon optimization.

Given the large number of gene sequences available for a wide variety of animal, plant and microbial species, it is possible to calculate the relative frequencies of codon usage (Nakamura, Y., et al. “Codon usage tabulated from the international DNA sequence databases: status for the year 2000” Nucl. Acids Res. 28:292 (2000)).

The term “flanking” refers to a relative position of one nucleic acid sequence with respect to another nucleic acid sequence. Generally, in the sequence ABC, B is flanked by A and C. The same is true for the arrangement A×B×C. Thus, a flanking sequence precedes or follows a flanked sequence but need not be contiguous with, or immediately adjacent to the flanked sequence. In one embodiment, the term flanking refers to terminal repeats at each end of the linear duplex ceDNA vector.

The term “exogenous” refers to a substance present in a cell other than its native source. The term “exogenous” when used herein can refer to a nucleic acid (e.g., a nucleic acid encoding a polypeptide) or a polypeptide that has been introduced by a process involving the hand of man into a bio-logical system such as a cell or organism in which it is not normally found and one wishes to intro-duce the nucleic acid or polypeptide into such a cell or organism. Alternatively, “exogenous” can refer to a nucleic acid or a polypeptide that has been introduced by a process involving the hand of man into a biological system such as a cell or organism in which it is found in relatively low amounts and one wishes to increase the amount of the nucleic acid or polypeptide in the cell or organism, e.g., to create ectopic expression or levels. In contrast, the term “endogenous” refers to a substance that is native to the biological system or cell.

The term “sequence identity” refers to the relatedness between two nucleotide sequences. For purposes of the present disclosure, the degree of sequence identity between two deoxyribonucleotide sequences is determined using the Needleman-Wunsch algorithm (Needleman and Wunsch, 1970, supra) as implemented in the Needle program of the EMBOSS package (EMBOSS: The European Molecular Biology Open Software Suite, Rice et al., 2000, supra), preferably version 3.0.0 or later. The optional parameters used are gap open penalty of 10, gap extension penalty of 0.5, and the EDNAFULL (EMBOSS version of NCBI NUC4.4) substitution matrix. The output of Needle labeled “longest identity” (obtained using the—nobrief option) is used as the percent identity and is calculated as follows: (Identical Deoxyribonucleotides.times.100)/(Length of Alignment-Total Number of Gaps in Alignment). The length of the alignment is preferably at least 10 nucleotides, preferably at least 25 nucleotides more preferred at least 50 nucleotides and most preferred at least 100 nucleotides.

As used herein, a “homology arm” refers to a polynucleotide that is suitable to target a donor sequence to a genome through homologous recombination. Typically, two homology arms flank the donor sequence, wherein each homology arm comprises genomic sequences upstream and down-stream of the locus of integration.

As used herein, “a donor sequence” refers to a polynucleotide that is to be inserted into, or used as a repair template for, a host cell genome. The donor sequence can comprise the modification which is desired to be made during gene editing. The sequence to be incorporated can be introduced into the target nucleic acid molecule via homology directed repair at the target sequence, thereby causing an alteration of the target sequence from the original target sequence to the sequence comprised by the donor sequence. Accordingly, the sequence comprised by the donor sequence can be, relative to the target sequence, an insertion, a deletion, an indel, a point mutation, a repair of a mutation, etc. The donor sequence can be, e.g., a single-stranded DNA molecule; a double-stranded DNA molecule; a DNA/RNA hybrid molecule; and a DNA/modRNA (modified RNA) hybrid molecule. In one embodiment, the donor sequence is foreign to the homology arms. The editing can be RNA as well as DNA editing. The donor sequence can be endogenous to or exogenous to the host cell genome, depending upon the nature of the desired gene editing.

“Heterologous,” as used herein, means a nucleotide or polypeptide sequence that is not found in the native nucleic acid or protein, respectively.

By “transformed cell” is meant a cell into which (or into an ancestor of which) has been introduced, by means of recombinant nucleic acid techniques, a nucleic acid molecule, i.e., a sequence of codons formed of nucleic acids (e.g., DNA or RNA) encoding a protein of interest. The introduced nucleic acid sequence may be present as an extrachromosomal or chromosomal element.

By “transformed cell” is meant a cell into which (or into an ancestor of which) has been introduced, by means of recombinant nucleic acid techniques, a nucleic acid molecule, i.e., a sequence of codons formed of nucleic acids (e.g., DNA or RNA) encoding a protein of interest. The introduced nucleic acid sequence may be present as an extrachromosomal or chromosomal element.

The terms “Correcting”, “genome editing” and “restoring” as used herein refers to changing a mutant gene that encodes a truncated protein or no protein at all, such that a full-length functional or partially full-length functional protein expression is obtained. Correcting or restoring a mutant gene may include replacing the region of the gene that has the mutation or replacing the entire mutant gene with a copy of the gene that does not have the mutation with a repair mechanism such as homology-directed repair (HDR). Correcting or restoring a mutant gene may also include repairing a frameshift mutation that causes a premature stop codon, an aberrant splice acceptor site or an aberrant splice donor site, by generating a double stranded break in the gene that is then repaired using non-homologous end joining (NHEJ). NHEJ may add or delete at least one base pair during repair which may restore the proper reading frame and eliminate the premature stop codon. Correcting or restoring a mutant gene may also include disrupting an aberrant splice acceptor site or splice donor sequence. Correcting or restoring a mutant gene may also include deleting a non-essential gene segment by the simultaneous action of two nucleases on the same DNA strand in order to restore the proper reading frame by removing the DNA between the two nuclease target sites and repairing the DNA break by NHEJ.

The phrase “Non-homologous end joining (NHEJ) pathway” as used herein refers to a pathway that repairs double-strand breaks in DNA by directly ligating the break ends without the need for a homologous template. The template-independent re-ligation of DNA ends by NHEJ is a stochastic, error-prone repair process that introduces random micro-insertions and micro-deletions (indels) at the DNA breakpoint. This method may be used to intentionally disrupt, delete, or alter the reading frame of targeted gene sequences. NHEJ typically uses short homologous DNA sequences called microhomologies to guide repair. These microhomologies are often present in single-stranded overhangs on the end of double-strand breaks. When the overhangs are perfectly compatible, NHEJ usually re-pairs the break accurately, yet imprecise repair leading to loss of nucleotides may also occur, but is much more common when the overhangs are not compatible “Nuclease mediated NHEJ” as used herein refers to NHEJ that is initiated after a nuclease, such as a cas9 or other nuclease, cuts double stranded DNA. In a CRISPR/CAS system NHEJ can be targeted by using a single guide RNA sequence.

“Homology-directed repair” or “HDR” as used interchangeably herein refers to a mechanism in cells to repair double strand DNA lesions when a homologous piece of DNA is present in the nucleus. HDR uses a donor DNA template to guide repair and may be used to create specific sequence changes to the genome, including the targeted addition of whole genes. If a donor template is provided along with the site specific nuclease, such as with a CRISPR/Cas9-based systems, then the cellular machinery will repair the break by homologous recombination, which is enhanced several orders of magnitude in the presence of DNA cleavage. When the homologous DNA piece is absent, non-homologous end joining may take place instead. In a CRISPR/Cas system one guide RNA, or two different guide RNAS can be used for HDR.

“Repeat variable diresidue” or “RVD” as used interchangeably herein refers to a pair of adjacent amino acid residues within a DNA recognition motif (also known as “RVD module”), which includes 33-35 amino acids, of a TALE DNA-binding domain. The RVD determines the nucleotide specificity of the RVD module. RVD modules may be combined to produce an RVD array. The “RVD array length” as used herein refers to the number of RVD modules that corresponds to the length of the nucleotide sequence within the TALEN target region that is recognized by a TALEN, i.e., the binding region.

“Site-specific nuclease” or “sequence specific nuclease” as used herein refers to an enzyme capable of specifically recognizing and cleaving DNA sequences. The site-specific nuclease may be engineered. Examples of engineered site-specific nucleases include zinc finger nucleases (ZFNs), TAL effector nucleases (TALENs), and CRISPR/Cas-based systems, that use various natural and unnatural Cas enzymes.

Other than in the operating examples, or where otherwise indicated, all numbers expressing quantities of ingredients or reaction conditions used herein should be understood as modified in all instances by the term “about.” The term “about” when used in connection with percentages can mean±1%.

Groupings of alternative elements or embodiments of the invention disclosed herein are not to be construed as limitations. Each group member can be referred to and claimed individually or in any combination with other members of the group or other elements found herein. One or more members of a group can be included in, or deleted from, a group for reasons of convenience and/or patentability. When any such inclusion or deletion occurs, the specification is herein deemed to contain the group as modified thus fulfilling the written description of all Markush groups used in the appended claims.

Unless otherwise defined herein, scientific and technical terms used in connection with the present application shall have the meanings that are commonly understood by those of ordinary skill in the art to which this disclosure belongs. It should be understood that this invention is not limited to the particular methodology, protocols, and reagents, etc., described herein and as such can vary. The terminology used herein is for the purpose of describing particular embodiments only, and is not intended to limit the scope of the present invention, which is defined solely by the claims. Definitions of common terms in immunology and molecular biology can be found in The Merck Manual of Diagnosis and Therapy, 19th Edition, published by Merck Sharp & Dohme Corp., 2011 (ISBN 978-0-911910-19-3); Robert S. Porter et al. (eds.), The Encyclopedia of Molecular Cell Biology and Molecular Medicine, published by Blackwell Science Ltd., 1999-2012 (ISBN 9783527600908); and Robert A. Meyers (ed.), Molecular Biology and Biotechnology: a Comprehensive Desk Reference, published by VCH Publishers, Inc., 1995 (ISBN 1-56081-569-8); Immunology by Werner Luttmann, published by Elsevier, 2006; Janeway's Immunobiology, Kenneth Murphy, Allan Mowat, Casey Weaver (eds.), Taylor & Francis Limited, 2014 (ISBN 0815345305, 9780815345305); Lewin's Genes XI, published by Jones & Bartlett Publishers, 2014 (ISBN-1449659055); Michael Richard Green and Joseph Sambrook, Molecular Cloning: A Laboratory Manual, 4^{th} ed., Cold Spring Harbor Laboratory Press, Cold Spring Harbor, N.Y., USA (2012) (ISBN 1936113414); Davis et al., Basic Methods in Molecular Biology, Elsevier Science Publishing, Inc., New York, USA (2012) (ISBN 044460149X); Laboratory Methods in Enzymology: DNA, Jon Lorsch (ed.) Elsevier, 2013 (ISBN 0124199542); Current Protocols in Molecular Biology (CPMB), Frederick M. Ausubel (ed.), John Wiley and Sons, 2014 (ISBN 047150338X, 9780471503385), Current Protocols in Protein Science (CPPS), John E. Coligan (ed.), John Wiley and Sons, Inc., 2005; and Current Protocols in Immunology (CPI) (John E. Coligan, ADA M Kruisbeek, David H Margulies, Ethan M Shevach, Warren Strobe, (eds.) John Wiley and Sons, Inc., 2003 (ISBN 0471142735, 9780471142737), the contents of which are all incorporated by reference herein in their entireties.

In some embodiments of any of the aspects, the disclosure described herein does not concern a process for cloning human beings, processes for modifying the germ line genetic identity of human beings, uses of human embryos for industrial or commercial purposes or processes for modifying the genetic identity of animals which are likely to cause them suffering without any substantial medical benefit to man or animal, and also animals resulting from such processes.

All patents and other publications; including literature references, issued patents, published patent applications, and co-pending patent applications; cited throughout this application are expressly incorporated herein by reference for the purpose of describing and disclosing, for example, the methodologies described in such publications that might be used in connection with the technology described herein. These publications are provided solely for their disclosure prior to the filing date of the present application. Nothing in this regard should be construed as an admission that the inventors are not entitled to antedate such disclosure by virtue of prior invention or for any other reason. All statements as to the date or representation as to the contents of these documents is based on the information available to the applicants and does not constitute any admission as to the correctness of the dates or contents of these documents.

The description of embodiments of the disclosure is not intended to be exhaustive or to limit the disclosure to the precise form disclosed. While specific embodiments of, and examples for, the disclosure are described herein for illustrative purposes, various equivalent modifications are possible within the scope of the disclosure, as those skilled in the relevant art will recognize. For example, while method steps or functions are presented in a given order, alternative embodiments may perform functions in a different order, or functions may be performed substantially concurrently. The teachings of the disclosure provided herein can be applied to other procedures or methods as appropriate. The various embodiments described herein can be combined to provide further embodiments. Aspects of the disclosure can be modified, if necessary, to employ the compositions, functions and concepts of the above references and application to provide yet further embodiments of the disclosure. Moreover, due to biological functional equivalency considerations, some changes can be made in protein structure without affecting the biological or chemical action in kind or amount. These and other changes can be made to the disclosure in light of the detailed description. All such modifications are intended to be included within the scope of the appended claims.

Specific elements of any of the foregoing embodiments can be combined or substituted for elements in other embodiments. Furthermore, while advantages associated with certain embodiments of the disclosure have been described in the context of these embodiments, other embodiments may also exhibit such advantages, and not all embodiments need necessarily exhibit such advantages to fall within the scope of the disclosure.

The technology described herein is further illustrated by the following examples which in no way should be construed as being further limiting.

### EXAMPLES

### Example 1: Constructing ceDNA Vectors for Insertion of a Transgene at a GSH Locus

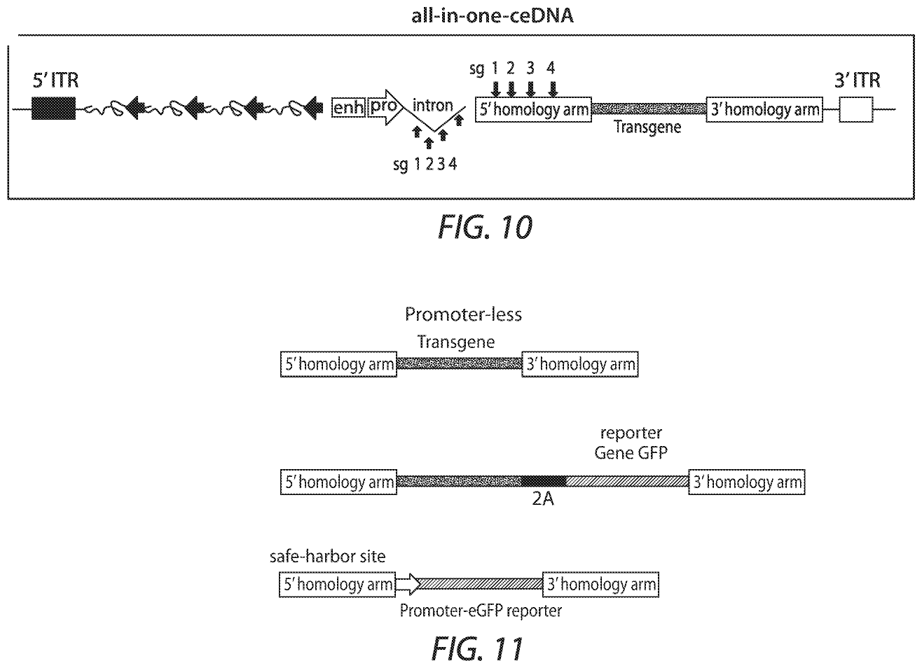

Exemplary ceDNA vectors with a 5′ GSH-specific homology arm and a 3′ GSH-specific homology arm are made with a 5′ GSH-specific homology arm (HA-L) and a 3′ GSH-specific homology arm (HA-R) that is specific to a GSH identified herein, e.g., Pax5 or a GSH identified in Table 1A or Table 1B. Exemplary ceDNA vectors are generated using ceDNA plasmids that comprise in this order: a first TR (e.g. a first ITR), a 5′ GSH-specific homology arm (i.e., a HA-L), a nucleic acid of interest (e.g. a therapeutic nucleic acid), a 3′ GSH-specific homology arm (a HA-R), and a second TR (e.g. a second ITR), where the first and second ITRs can be symmetrical, substantially symmetrical or asymmetrical relative to each other, as defined herein. Such ceDNA vectors can be administered with one or more gene editing molecules, including but not limited to The exemplary ceDNA vector shown in FIG. 1A can be administered with one or more vectors, including a ceDNA vector expressing a gene editing molecule, such as those described in International Patent Application PCT/US18/64242, which is incorporated herein in its entirety by reference. In some embodiments, the ceDNA plasmid may further comprise between the ITRs, but outside of the HA-L and HA-R region, a gene editing cassette, e.g., see FIG. 8 or FIG. 10, comprising one or more of a sgRNA expression unit and/or a nuclease expression unit, comprising one or more of, at least one guide RNA directed to the GSH, and a nuclease (e.g., Cas9) CRISPR/Cas, ZFN or Tale nucleic acid sequences. These plasmids produce the ceDNA vectors that target the GSH regions described herein, e.g. from Table 1A or 1B.

Production of the ceDNA vectors using a polynucleotide construct template is described in Example 1 of PCT/US18/49996, which is incorporated herein in its entirety by reference. Production of ceDNA vectors comprising a gene editing cassette are described in the Examples of International Application PCT/US/64242 filed on Dec. 6, 2018, which is incorporated herein in its entirety by reference. For example, a polynucleotide construct template used for generating the ceDNA vectors of the present invention can be a ceDNA-plasmid, a ceDNA-Bacmid, and/or a ceDNA-baculovirus. Without being limited to theory, in a permissive host cell, in the presence of e.g., Rep, the polynucleotide construct template having two symmetric ITRs and an expression construct, where at least one of the ITRs is modified relative to a wild-type ITR sequence, replicates to produce ceDNA vectors. ceDNA vector production undergoes two steps: first, excision (“rescue”) of template from the template backbone (e.g. ceDNA-plasmid, ceDNA-bacmid, ceDNA-baculovirus genome etc.) via Rep proteins, and second, Rep mediated replication of the excised ceDNA vector.

An exemplary method to produce ceDNA vectors is from a ceDNA-plasmid as described herein. Referring to FIGS. 1B and 1C, the polynucleotide construct template of each of the ceDNA-plasmids includes both a left modified ITR and a right modified ITR with the following between the ITR sequences: a HA-L, a (i) an enhancer/promoter; (ii) a cloning site for a transgene; (iii) a posttranscriptional response element (e.g. the woodchuck hepatitis virus posttranscriptional regulatory element (WPRE)); and (iv) a poly-adenylation signal (e.g. from bovine growth hormone gene (BGHpA), and a HA-R. Unique restriction endonuclease recognition sites (R1-R6) (shown in FIGS. 1B and 1C) were also introduced between each component to facilitate the introduction of new genetic components into the specific sites in the construct. R3 (PmeI) GTTTAAAC (SEQ ID NO: 123) and R4 (PacI) TTAATTAA (SEQ ID NO: 124) enzyme sites are engineered into the cloning site to introduce an open reading frame of a transgene. These sequences were cloned into a pFastBac HT B plasmid obtained from ThermoFisher Scientific.

Production of ceDNA-Bacmids:

DH10Bac competent cells (MAX EFFICIENCY® DH10Bac™ Competent Cells, Thermo Fisher) were transformed with either test or control plasmids following a protocol according to the manufacturer's instructions. Recombination between the plasmid and a baculovirus shuttle vector in the DH10Bac cells were induced to generate recombinant ceDNA-bacmids. The recombinant bacmids were selected by screening a positive selection based on blue-white screening in E. coli (Φ80dlacZΔM15 marker provides α-complementation of the β-galactosidase gene from the bacmid vector) on a bacterial agar plate containing X-gal and IPTG with antibiotics to select for transformants and maintenance of the bacmid and transposase plasmids. White colonies caused by transposition that disrupts the β-galactoside indicator gene were picked and cultured in 10 ml of media.

The recombinant ceDNA-bacmids were isolated from the E. coli and transfected into Sf9 or Sf21 insect cells using FugeneHD to produce infectious baculovirus. The adherent Sf9 or Sf21 insect cells were cultured in 50 ml of media in T25 flasks at 25° C. Four days later, culture medium (containing the P0 virus) was removed from the cells, filtered through a 0.45 μm filter, separating the infectious baculovirus particles from cells or cell debris.

Optionally, the first generation of the baculovirus (P0) was amplified by infecting naïve Sf9 or Sf21 insect cells in 50 to 500 ml of media. Cells were maintained in suspension cultures in an orbital shaker incubator at 130 rpm at 25° C., monitoring cell diameter and viability, until cells reach a diameter of 18-19 nm (from a naïve diameter of 14-15 nm), and a density of ˜4.0E+6 cells/mL. Between 3 and 8 days post-infection, the P1 baculovirus particles in the medium were collected following centrifugation to remove cells and debris then filtration through a 0.45 μm filter.

The ceDNA-baculovirus comprising the test constructs were collected and the infectious activity, or titer, of the baculovirus was determined. Specifically, four×20 ml Sf9 cell cultures at 2.5E+6 cells/ml were treated with P1 baculovirus at the following dilutions: 1/1000, 1/10,000, 1/50,000, 1/100,000, and incubated at 25-27° C. Infectivity was determined by the rate of cell diameter increase and cell cycle arrest, and change in cell viability every day for 4 to 5 days.

A “Rep-plasmid” was produced in a pFASTBAC™-Dual expression vector (ThermoFisher) comprising both the Rep78 (SEQ ID NO: 131 or 133) or Rep68 (SEQ ID NO: 130) and Rep52 (SEQ ID NO: 132) or Rep40 (SEQ ID NO: 129). The Rep-plasmid was transformed into the DH10Bac competent cells (MAX EFFICIENCY® DH10Bac™ Competent Cells (Thermo Fisher) following a protocol provided by the manufacturer. Recombination between the Rep-plasmid and a baculovirus shuttle vector in the DH10Bac cells were induced to generate recombinant bacmids (“Rep-bacmids”). The recombinant bacmids were selected by a positive selection that included-blue-white screening in E. coli (Φ80dlacZΔM15 marker provides α-complementation of the β-galactosidase gene from the bacmid vector) on a bacterial agar plate containing X-gal and IPTG. Isolated white colonies were picked and inoculated in 10 ml of selection media (kanamycin, gentamicin, tetracycline in LB broth). The recombinant bacmids (Rep-bacmids) were isolated from the E. coli and the Rep-bacmids were transfected into Sf9 or Sf21 insect cells to produce infectious baculovirus.

The Sf9 or Sf21 insect cells were cultured in 50 ml of media for 4 days, and infectious recombinant baculovirus (“Rep-baculovirus”) were isolated from the culture. Optionally, the first generation Rep-baculovirus (P0) were amplified by infecting naïve Sf9 or Sf21 insect cells and cultured in 50 to 500 ml of media. Between 3 and 8 days post-infection, the P1 baculovirus particles in the medium were collected either by separating cells by centrifugation or filtration or another fractionation process. The Rep-baculovirus were collected and the infectious activity of the baculovirus was determined. Specifically, four×20 mL Sf9 cell cultures at 2.5×10⁶ cells/mL were treated with P1 baculovirus at the following dilutions, 1/1000, 1/10,000, 1/50,000, 1/100,000, and incubated. Infectivity was determined by the rate of cell diameter increase and cell cycle arrest, and change in cell viability every day for 4 to 5 days.

ceDNA Vector Generation and Characterization

With reference to FIG. 4B, Sf9 insect cell culture media containing either (1) a sample-containing a ceDNA-bacmid or a ceDNA-baculovirus, and (2) Rep-baculovirus described above were then added to a fresh culture of Sf9 cells (2.5E+6 cells/ml, 20 ml) at a ratio of 1:1000 and 1:10,000, respectively. The cells were then cultured at 130 rpm at 25° C. 4-5 days after the co-infection, cell diameter and viability are detected. When cell diameters reached 18-20 nm with a viability of ˜70-80%, the cell cultures were centrifuged, the medium was removed, and the cell pellets were collected. The cell pellets are first resuspended in an adequate volume of aqueous medium, either water or buffer. The ceDNA vector was isolated and purified from the cells using Qiagen MIDI PLUS™ purification protocol (Qiagen, 0.2 mg of cell pellet mass processed per column).

Yields of ceDNA vectors produced and purified from the Sf9 insect cells were initially determined based on UV absorbance at 260 nm.

ceDNA vectors can be assessed by identified by agarose gel electrophoresis under native or denaturing conditions as illustrated in FIG. 4D, where (a) the presence of characteristic bands migrating at twice the size on denaturing gels versus native gels after restriction endonuclease cleavage and gel electrophoretic analysis and (b) the presence of monomer and dimer (2×) bands on denaturing gels for uncleaved material is characteristic of the presence of ceDNA vector.

Structures of the isolated ceDNA vectors were further analyzed by digesting the DNA obtained from co-infected Sf9 cells (as described herein) with restriction endonucleases selected for a) the presence of only a single cut site within the ceDNA vectors, and b) resulting fragments that were large enough to be seen clearly when fractionated on a 0.8% denaturing agarose gel (>800 bp). As illustrated in FIGS. 4D and 4E, linear DNA vectors with a non-continuous structure and ceDNA vector with the linear and continuous structure can be distinguished by sizes of their reaction products—for example, a DNA vector with a non-continuous structure is expected to produce 1 kb and 2 kb fragments, while a non-encapsidated vector with the continuous structure is expected to produce 2 kb and 4 kb fragments.

Therefore, to demonstrate in a qualitative fashion that isolated ceDNA vectors are covalently closed-ended as is required by definition, the samples were digested with a restriction endonuclease identified in the context of the specific DNA vector sequence as having a single restriction site, preferably resulting in two cleavage products of unequal size (e.g., 1000 bp and 2000 bp). Following digestion and electrophoresis on a denaturing gel (which separates the two complementary DNA strands), a linear, non-covalently closed DNA will resolve at sizes 1000 bp and 2000 bp, while a covalently closed DNA (i.e., a ceDNA vector) will resolve at 2× sizes (2000 bp and 4000 bp), as the two DNA strands are linked and are now unfolded and twice the length (though single stranded). Furthermore, digestion of monomeric, dimeric, and n-meric forms of the DNA vectors will all resolve as the same size fragments due to the end-to-end linking of the multimeric DNA vectors (see FIG. 4D).

As used herein, the phrase “assay for the Identification of DNA vectors by agarose gel electrophoresis under native gel and denaturing conditions” refers to an assay to assess the close-endedness of the ceDNA by performing restriction endonuclease digestion followed by electrophoretic assessment of the digest products. One such exemplary assay follows, though one of ordinary skill in the art will appreciate that many art-known variations on this example are possible. The restriction endonuclease is selected to be a single cut enzyme for the ceDNA vector of interest that will generate products of approximately ⅓× and ⅔× of the DNA vector length. This resolves the bands on both native and denaturing gels. Before denaturation, it is important to remove the buffer from the sample. The Qiagen PCR clean-up kit or desalting “spin columns,” e.g. GE HEALTHCARE ILUSTRA™ MICROSPIN™ G-25 columns are some art-known options for the endonuclease digestion. The assay includes for example, i) digest DNA with appropriate restriction endonuclease(s), 2) apply to e.g., a Qiagen PCR clean-up kit, elute with distilled water, iii) adding 10× denaturing solution (10×=0.5 M NaOH, 10 mM EDTA), add 10× dye, not buffered, and analyzing, together with DNA ladders prepared by adding 10× denaturing solution to 4×, on a 0.8-1.0% gel previously incubated with 1 mM EDTA and 200 mM NaOH to ensure that the NaOH concentration is uniform in the gel and gel box, and running the gel in the presence of 1× denaturing solution (50 mM NaOH, 1 mM EDTA). One of ordinary skill in the art will appreciate what voltage to use to run the electrophoresis based on size and desired timing of results. After electrophoresis, the gels are drained and neutralized in 1× TBE or TAE and transferred to distilled water or 1× TBE/TAE with 1× SYBR Gold. Bands can then be visualized with e.g. Thermo Fisher, SYBR® Gold Nucleic Acid Gel Stain (10,000× Concentrate in DMSO) and epifluorescent light (blue) or UV (312 nm).

The purity of the generated ceDNA vector can be assessed using any art-known method. As one exemplary and non-limiting method, contribution of ceDNA-plasmid to the overall UV absorbance of a sample can be estimated by comparing the fluorescent intensity of ceDNA vector to a standard. For example, if based on UV absorbance 4 μg of ceDNA vector was loaded on the gel, and the ceDNA vector fluorescent intensity is equivalent to a 2 kb band which is known to be 1 μg, then there is 1 μg of ceDNA vector, and the ceDNA vector is 25% of the total UV absorbing material. Band intensity on the gel is then plotted against the calculated input that band represents—for example, if the total ceDNA vector is 8 kb, and the excised comparative band is 2 kb, then the band intensity would be plotted as 25% of the total input, which in this case would be 0.25 μg for 1.0 μg input. Using the ceDNA vector plasmid titration to plot a standard curve, a regression line equation is then used to calculate the quantity of the ceDNA vector band, which can then be used to determine the percent of total input represented by the ceDNA vector, or percent purity.

For illustrative purposes, Example 2 describes the production of ceDNA vectors using an insect cell based method and a polynucleotide construct template, and is also described in Example 1 of PCT/US18/49996, which is incorporated herein in its entirety by reference. For example, a polynucleotide construct template used for generating the ceDNA vectors of the present invention according to Example 1 can be a ceDNA-plasmid, a ceDNA-Bacmid, and/or a ceDNA-baculovirus. Without being limited to theory, in a permissive host cell, in the presence of e.g., Rep, the polynucleotide construct template having two symmetric ITRs and an expression construct, where at least one of the ITRs is modified relative to a wild-type ITR sequence, replicates to produce ceDNA vectors. ceDNA vector production undergoes two steps: first, excision (“rescue”) of template from the template backbone (e.g. ceDNA-plasmid, ceDNA-bacmid, ceDNA-baculovirus genome etc.) via Rep proteins, and second, Rep mediated replication of the excised ceDNA vector.

An exemplary method to produce ceDNA vectors in a method using insect cell is from a ceDNA-plasmid as described herein. Referring to FIGS. 1B and 1C, the polynucleotide construct template of each of the ceDNA-plasmids includes both a left 5′ ITR and a right 3′ ITR with the following between the ITR sequences: a HA-L and a HA-R, and located between the HA-L and HA-R, the following (i) an enhancer/promoter; (ii) a cloning site for a transgene; (iii) a posttranscriptional response element (e.g. the woodchuck hepatitis virus posttranscriptional regulatory element (WPRE)); and (iv) a poly-adenylation signal (e.g. from bovine growth hormone gene (BGHpA). Unique restriction endonuclease recognition sites (R1-R6) (shown in FIG. 1B and FIG. 1C) were also introduced between each component to facilitate the introduction of new genetic components into the specific sites in the construct. R3 (PmeI) GTTTAAAC (SEQ ID NO: 123) and R4 (PacI) TTAATTAA (SEQ ID NO: 124) enzyme sites are engineered into the cloning site to introduce an open reading frame of a transgene. These sequences were cloned into a pFastBac HT B plasmid obtained from ThermoFisher Scientific.

Production of ceDNA-Bacmids:

DH10Bac competent cells (MAX EFFICIENCY® DH10Bac™ Competent Cells, Thermo Fisher) were transformed with either test or control plasmids following a protocol according to the manufacturer's instructions. Recombination between the plasmid and a baculovirus shuttle vector in the DH10Bac cells were induced to generate recombinant ceDNA-bacmids. The recombinant bacmids were selected by screening a positive selection based on blue-white screening in E. coli (Φ80dlacZΔM15 marker provides α-complementation of the β-galactosidase gene from the bacmid vector) on a bacterial agar plate containing X-gal and IPTG with antibiotics to select for transformants and maintenance of the bacmid and transposase plasmids. White colonies caused by transposition that disrupts the β-galactoside indicator gene were picked and cultured in 10 ml of media.

The recombinant ceDNA-bacmids were isolated from the E. coli and transfected into Sf9 or Sf21 insect cells using FugeneHD to produce infectious baculovirus. The adherent Sf9 or Sf21 insect cells were cultured in 50 ml of media in T25 flasks at 25° C. Four days later, culture medium (containing the P0 virus) was removed from the cells, filtered through a 0.45 μm filter, separating the infectious baculovirus particles from cells or cell debris.

Optionally, the first generation of the baculovirus (P0) was amplified by infecting naïve Sf9 or Sf21 insect cells in 50 to 500 ml of media. Cells were maintained in suspension cultures in an orbital shaker incubator at 130 rpm at 25° C., monitoring cell diameter and viability, until cells reach a diameter of 18-19 nm (from a naïve diameter of 14-15 nm), and a density of ˜4.0E+6 cells/mL. Between 3 and 8 days post-infection, the P1 baculovirus particles in the medium were collected following centrifugation to remove cells and debris then filtration through a 0.45 μm filter.

The ceDNA-baculovirus comprising the test constructs were collected and the infectious activity, or titer, of the baculovirus was determined. Specifically, four×20 ml Sf9 cell cultures at 2.5E+6 cells/ml were treated with P1 baculovirus at the following dilutions: 1/1000, 1/10,000, 1/50,000, 1/100,000, and incubated at 25-27° C. Infectivity was determined by the rate of cell diameter increase and cell cycle arrest, and change in cell viability every day for 4 to 5 days.

A “Rep-plasmid” was produced in a pFASTBAC™-Dual expression vector (ThermoFisher) comprising both the Rep78 (SEQ ID NO: 131 or 133) or Rep68 (SEQ ID NO: 130) and Rep52 (SEQ ID NO: 132) or Rep40 (SEQ ID NO: 129). The Rep-plasmid was transformed into the DH10Bac competent cells (MAX EFFICIENCY® DH10Bac™ Competent Cells (Thermo Fisher) following a protocol provided by the manufacturer. Recombination between the Rep-plasmid and a baculovirus shuttle vector in the DH10Bac cells were induced to generate recombinant bacmids (“Rep-bacmids”). The recombinant bacmids were selected by a positive selection that included-blue-white screening in E. coli (Φ80dlacZΔM15 marker provides α-complementation of the β-galactosidase gene from the bacmid vector) on a bacterial agar plate containing X-gal and IPTG. Isolated white colonies were picked and inoculated in 10 ml of selection media (kanamycin, gentamicin, tetracycline in LB broth). The recombinant bacmids (Rep-bacmids) were isolated from the E. coli and the Rep-bacmids were transfected into Sf9 or Sf21 insect cells to produce infectious baculovirus.

The Sf9 or Sf21 insect cells were cultured in 50 ml of media for 4 days, and infectious recombinant baculovirus (“Rep-baculovirus”) were isolated from the culture. Optionally, the first generation Rep-baculovirus (P0) were amplified by infecting naïve Sf9 or Sf21 insect cells and cultured in 50 to 500 ml of media. Between 3 and 8 days post-infection, the P1 baculovirus particles in the medium were collected either by separating cells by centrifugation or filtration or another fractionation process. The Rep-baculovirus were collected and the infectious activity of the baculovirus was determined. Specifically, four×20 mL Sf9 cell cultures at 2.5×10⁶ cells/mL were treated with P1 baculovirus at the following dilutions, 1/1000, 1/10,000, 1/50,000, 1/100,000, and incubated. Infectivity was determined by the rate of cell diameter increase and cell cycle arrest, and change in cell viability every day for 4 to 5 days.

ceDNA Vector Generation and Characterization

Sf9 insect cell culture media containing either (1) a sample-containing a ceDNA-bacmid or a ceDNA-baculovirus, and (2) Rep-baculovirus described above were then added to a fresh culture of Sf9 cells (2.5E+6 cells/ml, 20 ml) at a ratio of 1:1000 and 1:10,000, respectively. The cells were then cultured at 130 rpm at 25° C. 4-5 days after the co-infection, cell diameter and viability are detected. When cell diameters reached 18-20 nm with a viability of ˜70-80%, the cell cultures were centrifuged, the medium was removed, and the cell pellets were collected. The cell pellets are first resuspended in an adequate volume of aqueous medium, either water or buffer. The ceDNA vector was isolated and purified from the cells using Qiagen MIDI PLUS™ purification protocol (Qiagen, 0.2 mg of cell pellet mass processed per column).

Yields of ceDNA vectors produced and purified from the Sf9 insect cells were initially determined based on UV absorbance at 260 nm. The purified ceDNA vectors can be assessed for proper closed-ended configuration using the electrophoretic methodology described in Example 5.

### Example 2: Synthetic ceDNA Production Via Excision from a Double-Stranded DNA Molecule

Synthetic production of the ceDNA vectors is described in Examples 2-6 of International Application PCT/US19/14122, filed Jan. 18, 2019, which is incorporated herein in its entirety by reference. One exemplary method of producing a ceDNA vector using a synthetic method that involves the excision of a double-stranded DNA molecule. In brief, a ceDNA vector can be generated using a double stranded DNA construct, e.g., see FIGS. 7A-8E of PCT/US19/14122. In some embodiments, the double stranded DNA construct is a ceDNA plasmid, e.g., see, e.g., FIG. 6 in International patent application PCT/US2018/064242, filed Dec. 6, 2018).

In some embodiments, a construct to make a ceDNA vector comprises a regulatory switch as described herein.

For illustrative purposes, Example 2 describes producing ceDNA vectors as exemplary closed-ended DNA vectors generated using this method. However, while ceDNA vectors are exemplified in this Example to illustrate in vitro synthetic production methods to generate a closed-ended DNA vector by excision of a double-stranded polynucleotide comprising the ITRs and expression cassette (e.g., heterologous nucleic acid sequence) followed by ligation of the free 3′ and 5′ ends as described herein, one of ordinary skill in the art is aware that one can, as illustrated above, modify the double stranded DNA polynucleotide molecule such that any desired closed-ended DNA vector is generated, including but not limited to, doggybone DNA, dumbbell DNA and the like.

The method involves (i) excising a sequence encoding the expression cassette from a double-stranded DNA construct and (ii) forming hairpin structures at one or more of the ITRs and (iii) joining the free 5′ and 3′ ends by ligation, e.g., by T4 DNA ligase.

The double-stranded DNA construct comprises, in 5′ to 3′ order: a first restriction endonuclease site; an upstream ITR; a HA-L, an expression cassette; a HA-R a downstream ITR; and a second restriction endonuclease site. The double-stranded DNA construct is then contacted with one or more restriction endonucleases to generate double-stranded breaks at both of the restriction endonuclease sites. One endonuclease can target both sites, or each site can be targeted by a different endonuclease as long as the restriction sites are not present in the ceDNA vector template. This excises the sequence between the restriction endonuclease sites from the rest of the double-stranded DNA construct. Upon ligation a closed-ended DNA vector is formed.

One or both of the ITRs used in the method may be wild-type ITRs. Modified ITRs may also be used, where the modification can include deletion, insertion, or substitution of one or more nucleotides from the wild-type ITR in the sequences forming B and B′ arm and/or C and C′ arm, and may have two or more hairpin loops or a single hairpin loop. The hairpin loop modified ITR can be generated by genetic modification of an existing oligo or by de novo biological and/or chemical synthesis.

In a non-limiting example, ITR-6 Left and Right (SEQ ID NOS: 111 and 112), include 40 nucleotide deletions in the B-B′ and C-C′ arms from the wild-type ITR of AAV2. Nucleotides remaining in the modified ITR are predicted to form a single hairpin structure. Gibbs free energy of unfolding the structure is about ˜54.4 kcal/mol. Other modifications to the ITR may also be made, including optional deletion of a functional Rep binding site or a Trs site.

### Example 3: ceDNA Production Via Oligonucleotide Construction

Another exemplary method of producing a ceDNA vector using a synthetic method that involves assembly of various oligonucleotides, is provided in Example 3 of PCT/US19/14122, where a ceDNA vector is produced by synthesizing a 5′ oligonucleotide and a 3′ ITR oligonucleotide and ligating the ITR oligonucleotides to a double-stranded polynucleotide comprising an expression cassette. FIG. 11B of PCT/US19/14122 shows an exemplary method of ligating a 5′ ITR oligonucleotide and a 3′ ITR oligonucleotide to a double stranded polynucleotide comprising an expression cassette.

As disclosed herein, the ITR oligonucleotides can comprise WT-ITRs or modified ITRs (e.g., see, FIGS. 6A, 6B, 7A and 7B of PCT/US19/14122, which is incorporated herein in its entirety). Exemplary ITR oligonucleotides include, but are not limited to SEQ ID NOS: 134-145 (e.g., see Table 7 in of PCT/US19/14122). Modified ITRs can include deletion, insertion, or substitution of one or more nucleotides from the wild-type ITR in the sequences forming B and B′ arm and/or C and C′ arm. ITR oligonucleotides, comprising WT-ITRs or mod-ITRs as described herein, to be used in the cell-free synthesis, can be generated by genetic modification or biological and/or chemical synthesis. As discussed herein, the ITR oligonucleotides in Examples 3 and 4 can comprise WT-ITRs, or modified ITRs (mod-ITRs) in symmetrical or asymmetrical configurations, as discussed herein.

### Example 4: ceDNA Production Via a Single-Stranded DNA Molecule

Another exemplary method of producing a ceDNA vector using a synthetic method is provided in Example 4 of PCT/US19/14122, and uses a single-stranded linear DNA comprising two sense ITRs which flank a sense expression cassette sequence and are attached covalently to two antisense ITRs which flank an antisense expression cassette, the ends of which single stranded linear DNA are then ligated to form a closed-ended single-stranded molecule. One non-limiting example comprises synthesizing and/or producing a single-stranded DNA molecule, annealing portions of the molecule to form a single linear DNA molecule which has one or more base-paired regions of secondary structure, and then ligating the free 5′ and 3′ ends to each other to form a closed single-stranded molecule.

An exemplary single-stranded DNA molecule for production of a ceDNA vector comprises, from 5′ to 3′:
a sense first ITR;
a sense HA-L
a sense expression cassette sequence;
a sense HA-R
a sense second ITR;
an antisense second ITR;
an antisense HA-R
an antisense expression cassette sequence;
an antisense HA-L and
an antisense first ITR.

A single-stranded DNA molecule for use in the exemplary method of Example 4 can be formed by any DNA synthesis methodology described herein, e.g., in vitro DNA synthesis, or provided by cleaving a DNA construct (e.g., a plasmid) with nucleases and melting the resulting dsDNA fragments to provide ssDNA fragments.

Annealing can be accomplished by lowering the temperature below the calculated melting temperatures of the sense and antisense sequence pairs. The melting temperature is dependent upon the specific nucleotide base content and the characteristics of the solution being used, e.g., the salt concentration. Melting temperatures for any given sequence and solution combination are readily calculated by one of ordinary skill in the art.

The free 5′ and 3′ ends of the annealed molecule can be ligated to each other, or ligated to a hairpin molecule to form the ceDNA vector. Suitable exemplary ligation methodologies and hairpin molecules are described in Examples 2 and 3.

### Example 5: Purifying and/or Confirming Production of ceDNA

Any of the DNA vector products produced by the methods described herein, e.g., including the insect cell based production methods described in Example 1, or synthetic production methods described in Examples 2-4 can be purified, e.g., to remove impurities, unused components, or byproducts using methods commonly known by a skilled artisan; and/or can be analyzed to confirm that DNA vector produced, (in this instance, a ceDNA vector) is the desired molecule. An exemplary method for purification of the DNA vector, e.g., ceDNA is using Qiagen Midi Plus purification protocol (Qiagen) and/or by gel purification,

The following is an exemplary method for confirming the identity of ceDNA vectors.

ceDNA vectors can be assessed by identified by agarose gel electrophoresis under native or denaturing conditions as illustrated in FIGS. 4D and 4E, where (a) the presence of characteristic bands migrating at twice the size on denaturing gels versus native gels after restriction endonuclease cleavage and gel electrophoretic analysis and (b) the presence of monomer and dimer (2×) bands on denaturing gels for uncleaved material is characteristic of the presence of ceDNA vector.

Structures of the isolated ceDNA vectors were further analyzed by digesting the purified DNA with restriction endonucleases selected for a) the presence of only a single cut site within the ceDNA vectors, and b) resulting fragments that were large enough to be seen clearly when fractionated on a 0.8% denaturing agarose gel (>800 bp). As illustrated in FIGS. 4D and 4E, linear DNA vectors with a non-continuous structure and ceDNA vector with the linear and continuous structure can be distinguished by sizes of their reaction products—for example, a DNA vector with a non-continuous structure is expected to produce 1 kb and 2 kb fragments, while a ceDNA vector with the continuous structure is expected to produce 2 kb and 4 kb fragments.

Therefore, to demonstrate in a qualitative fashion that isolated ceDNA vectors are covalently closed-ended as is required by definition, the samples were digested with a restriction endonuclease identified in the context of the specific DNA vector sequence as having a single restriction site, preferably resulting in two cleavage products of unequal size (e.g., 1000 bp and 2000 bp). Following digestion and electrophoresis on a denaturing gel (which separates the two complementary DNA strands), a linear, non-covalently closed DNA will resolve at sizes 1000 bp and 2000 bp, while a covalently closed DNA (i.e., a ceDNA vector) will resolve at 2× sizes (2000 bp and 4000 bp), as the two DNA strands are linked and are now unfolded and twice the length (though single stranded). Furthermore, digestion of monomeric, dimeric, and n-meric forms of the DNA vectors will all resolve as the same size fragments due to the end-to-end linking of the multimeric DNA vectors (see FIGS. 4E and 4F).

As used herein, the phrase “assay for the Identification of DNA vectors by agarose gel electrophoresis under native gel and denaturing conditions” refers to an assay to assess the close-endedness of the ceDNA by performing restriction endonuclease digestion followed by electrophoretic assessment of the digest products. One such exemplary assay follows, though one of ordinary skill in the art will appreciate that many art-known variations on this example are possible. The restriction endonuclease is selected to be a single cut enzyme for the ceDNA vector of interest that will generate products of approximately ⅓× and ⅔× of the DNA vector length. This resolves the bands on both native and denaturing gels. Before denaturation, it is important to remove the buffer from the sample. The Qiagen PCR clean-up kit or desalting “spin columns,” e.g. GE HEALTHCARE ILUSTRA™ MICROSPIN™ G-25 columns are some art-known options for the endonuclease digestion. The assay includes for example, i) digest DNA with appropriate restriction endonuclease(s), 2) apply to e.g., a Qiagen PCR clean-up kit, elute with distilled water, iii) adding 10× denaturing solution (10×=0.5 M NaOH, 10 mM EDTA), add 10× dye, not buffered, and analyzing, together with DNA ladders prepared by adding 10× denaturing solution to 4×, on a 0.8-1.0% gel previously incubated with 1 mM EDTA and 200 mM NaOH to ensure that the NaOH concentration is uniform in the gel and gel box, and running the gel in the presence of 1× denaturing solution (50 mM NaOH, 1 mM EDTA). One of ordinary skill in the art will appreciate what voltage to use to run the electrophoresis based on size and desired timing of results. After electrophoresis, the gels are drained and neutralized in 1× TBE or TAE and transferred to distilled water or 1× TBE/TAE with 1× SYBR Gold. Bands can then be visualized with e.g. Thermo Fisher, SYBR® Gold Nucleic Acid Gel Stain (10,000× Concentrate in DMSO) and epifluorescent light (blue) or UV (312 nm). The foregoing gel-based method can be adapted to purification purposes by isolating the ceDNA vector from the gel band and permitting it to renature.

The purity of the generated ceDNA vector can be assessed using any art-known method. As one exemplary and non-limiting method, contribution of ceDNA-plasmid to the overall UV absorbance of a sample can be estimated by comparing the fluorescent intensity of ceDNA vector to a standard. For example, if based on UV absorbance 4 μg of ceDNA vector was loaded on the gel, and the ceDNA vector fluorescent intensity is equivalent to a 2 kb band which is known to be 1 μg, then there is 1 μg of ceDNA vector, and the ceDNA vector is 25% of the total UV absorbing material. Band intensity on the gel is then plotted against the calculated input that band represents—for example, if the total ceDNA vector is 8 kb, and the excised comparative band is 2 kb, then the band intensity would be plotted as 25% of the total input, which in this case would be 0.25 μg for 1.0 μg input. Using the ceDNA vector plasmid titration to plot a standard curve, a regression line equation is then used to calculate the quantity of the ceDNA vector band, which can then be used to determine the percent of total input represented by the ceDNA vector, or percent purity.

### Example 6: ceDNA Vectors with a 5′- and 3′ GSH-Specific Homology Arms Express a Transgene or Nucleic Acid of Interest In Vivo

In vivo protein expressions from ceDNA vectors described above are determined in mice. A nucleic acid of interest (i.e., transgene) with an open reading frame and any regulatory sequences is inserted into the ceDNA vector, flanked by 5′- and 3′ GSH-specific homology arms which bind to a GSH identified herein, e.g., in Tables 1A and 1B to facilitate HDR within the GSH loci. In some embodiments, the 5′- and 3′ GSH-specific homology arms are between 500-800 bp, or 800-2 kb, or larger than 2 kb. In experiments, a ceDNA vector comprises a nucleic acid encoding a nuclease, and the transgene to be inserted encodes a reporter protein with an open reading frame located between the HA-L and HA-R, and is administered to a subject or host cell along with any needed adjunct components such as sgRNA, with the nuclease specific for a site at or near the GSH locus and effective to increase recombination. In experiments, the ceDNA can delivered in lipid nanoparticles (LNPs) as described herein.

An exemplary test ceDNA vector expression unit can be assessed in accordance with the present disclosure, where the nucleic acid of interest is flanked by 5′ and 3′ GSH-specific homology arms complementary to, or substantially complementary to the GSH to allow for homologous recombination, where the 5′ and 3′ GSH-specific homology arms are incorporated into the TTX-1 a ceDNA design (FIG. 7).

In some embodiments, negative controls can be established, e.g., where a negative control ceDNA vector comprises either scrambled 3′- and/or 5′-GSH homology arms, or no homology arms, or alternatively, only a 5′- or 3′-GSH-specific homology arm (i.e., not both), where these negative control ceDNA vectors can be used to check for, and serve as negative controls for effective targeting of another ceDNA vector with 3′- and 5′-GSH-specific homology arms flanking a nucleic acid of interest. A nucleic acid of interest, or an expression unit, can be a marker gene, (also referred to herein as a reporter gene), e.g., GFP, including a promoter, WPRE element, pA, can be used to experimentally confirm expression.

In some embodiments, validation of the GSH by insertion of a nucleic acid of interest using a ceDNA vector described herein can also be performed by assessing off-target sites, and/or using next generation sequencing with tag-specific sequences that amplify the GSH locus with an inserted transgene or reporter gene. Such analysis is useful for assessing specificity and/or efficiency of targeting a GSH locus with a vector with 3′- and 5-GSH specific homology arms.

A nuclease expressing unit can be delivered in trans, such Cas9 mRNA, zinc-finger nucleases (ZFN), transcription activator-like effector nucleases (TALEN), mutated “nickase” endonuclease, class II CRISPR/Cas system (CPF1). In experiments, LNPs can be used as a delivery option. The transport into the nuclei can be increased by using a nuclear localization signal (NLS) fused into the 5′ or 3′ enzyme peptide sequence, according to methods commonly known to persons of ordinary skill in the art. In another embodiment, the NLS can be inserted internally such that the NLS is exposed on the surface of the nuclease and does not interfere with its function as a nuclease.

Where appropriate for the nuclease, to induce double-stranded break (DSB) at the desired site one or more single guided RNA are delivered in trans as well; Either as an sgRNA expressing ceDNA vector or chemically synthesized synthetic sgRNA. (sgRNA=single guide-RNA target sequence) as described herein. sgRNA can be selected using freely available software/algorithm, e.g., such as at tools.genome-engineering.org, can be used to select suitable single guide-RNA sequences.

The 5′ GSH-specific homology arm can be approximately 350 bp long, and can be in range between 50 to 2000 bp, as described herein. In some embodiments, the 3′ GSH-specific homology arm can be the same length or longer or shorter than the 5′ GSH-specific homology arm, and can be approximately 2000 bp long, or in the range of between 50 to 2000 bp, as described herein. Details study regarding length of homology arms and recombination frequency is e.g., reported by Jian-Ping Zhang et al., Genome Biology, 2017.

In further experiments, a therapeutic nucleic acid of interest ORF is substituted. In experiments, WPRE and polyadenylation signal, such as BGHpA can be added. In experiments, expression can also be regulated by the endogenous promoter of the GSH. In alternative embodiments, the promoter is a very strong promoter. In experiments, a translation enhancing element, such as WPRE is added 3′ of the ORF. In experiments, also, a polyadenylation signal (e.g., BGH-pA) is added needed as well.

Importantly, the capacity of the ceDNA vector, the length of the DNA fragment between the ITRs can be above 15 kb. Therefore, large HA-L and HA-R with a transgene with an ORFs are envisioned for use. In some embodiments, the GSH locus is PAX5 or KIF6 or any GSH listed in Table 1A or 1B. It is envisioned that one can insert into an intron site or exon in any of the regions disclosed in Table 1A or 1B can occur without any effects on the target cell or tissue.

### Example 7: All in One Vector

In some embodiments, expression constructs are made for titration of self-inactivating features of the nuclease activity by introducing sgRNA sequences in the intron of the synthetic promoter unit, e.g., the CAG promoter described herein. The degree of inactivation is determined by the number of sgRNA seq or combination and/or mutated (de-optimized) sgRNA target seq. (Zhang et al, NatPro, 2013 Regulation of Cas9 activity by using de-optimized sgRNA recognition target sequence.)

Master-ORF Expressing-All-in-One ceDNA Vector

In some embodiments, a ceDNA vector is made containing a nuclease expression unit (including hashed nuclease element) and an intron downstream of the promoter having the illustrated sgRNA targeting sequence. An exemplary vector is shown in FIG. 8 and FIG. 10. The features can include, but are not limited to, a ceDNA specific ITR; Pol III promoter (U6 or H1) driven sgRNA expressing unit with optional orientation in regard the transcription direction; Synthetic promoter driven nuclease (e.g., Cas9, double mutant Nickase, Talen, or other mutants) expression unit that may contain sgRNA targeting sequences with or w/o de-optimization (in experiments, located other than as indicated); A nucleic acid of interest (e.g., a transgene) potentially fused to a selection marker (e.g., NeoR or reporter protein, e.g., luciferase (SEQ ID NO: 56) through a viral 2A peptide cleavage site (2A) flanked by 0.05 to 6 kb stretching homology arms. (On 2A systems: Chan et al, Comparison of IRES and F2A-Based Locus-Specific Multicistronic Expression in Stable Mouse LinesHSV-TK suicide, PLOS 2011 HSV-TK suicide gene system; Fesnak et al, Engineered T Cells: The Promise and Challenges of Cancer Immunotherapy, NatRevCan 2016.) If suitable, a negative selection marker (e.g., HSV TK) and expressing unit that allows to control and select for successful integration into the GSH can be positioned inside of the 5′- and 3′ GSH-specific homology arms.

The 5′- and 3′ GSH-specific homology arms in the ceDNA vector allow for an anticipated site of insertion by homologous recombination. However, if instead there is random integration, the entire ceDNA vector with negative selectable marker is integrated into the genome. Such mis-transfected cells can be killed with appropriate drugs, such as GVC for the HSV TK negative selectable marker. In some embodiments, a negative selection marker can be replaced with a sgRNA target sequence for a “double mutant nickase” where the introduction of single stranded DNA cut (nicking) can help to release torsion downstream of the 3′ GSH-specific homology arm and increase annealing and therefore increase HDR frequency. In experiments, the negative marker is used with the sgRNA target sequence for “double mutant nickase.”

### REFERENCES

Publications and references, including but not limited to patents and patent applications, cited in this specification are herein incorporated by reference in their entirety in the entire portion cited as if each individual publication or reference were specifically and individually indicated to be incorporated by reference herein as being fully set forth. Any patent application to which this application claims priority is also incorporated by reference herein in the manner described above for publications and references.

1. A capsid free, linear, closed-ended DNA (ceDNA) vector comprising two inverted terminal repeats (ITRs), and located between the two ITRs, at least one heterologous nucleotide sequence, and at least one Genomic Safe Harbor Homology Arm (GSH HA), wherein the GSH HA binds to a target site located in a genomic safe harbor locus (GSH locus) in Table 1A or Table 1B, and wherein the GSH HA guides insertion of the heterologous nucleotide sequence into a locus located within the genomic safe harbor.

2. The ceDNA vector of claim 1, wherein the ceDNA comprises at least a 5′ Genomic Safe Harbor Homology Arm (5′ GSH HA) or a 3′ Genomic Safe Harbor Homology Arm (3′ GSH HA), or both, wherein the 5′ GSH HA and the 3′ GSH HA bind to a target site located in a genomic safe harbor locus (GSH locus) in Table 1A or Table 1B, and wherein the 5′ GSH HA and/or the 3′ GSH HA guide insertion of the heterologous nucleotide sequence into a locus located within the genomic safe harbor.

3. The ceDNA vector of claim 2, wherein the heterologous nucleotide sequence is 3′ of the 5′ GSH HA, or 5′ of the 3′ GSH HA.

4. The ceDNA vector of claim 2, wherein the heterologous nucleotide sequence is located between the 5′ GSH HA and the 3′ GSH HA.

5. The ceDNA vector of claim 1, wherein insertion is by homologous recombination, homology direct repair (HDR), or non-homologous end joining (NHEJ).

6. The ceDNA vector of claim 1, wherein the at least a portion of the GSH locus comprises the PAX5 genomic DNA or a fragment thereof.

7. The ceDNA vector of claim 1, wherein the GSH locus is an untranslated sequence or an intron or exon of the PAX5 gene.

8. The ceDNA vector of claim 1, wherein the target site is in the PAX5 GSH locus or KIF6, and is a region of at least 100-1000 nucleotides located in Chromosome 9 (36,833,275-37,034,185 reverse strand) or Chromosome 6 (39,329,990-39,725,405).

9. The ceDNA vector of claim 1, wherein the GSH locus is a nucleic acid selected from any of the nucleic acid sequences listed in Table 1A or 1B.

10. The ceDNA vector of claim 1, wherein the GSH locus is a region in any of the untranslated sequence or an intron or exon of the genes selected from Kif6, KLHL7, NUPL2, mir684, KCNH2, GPNMB, MIR4540, MIR4475, MIR4476, PRL32P21, LOC105376031, LOC105376032, LOC105376030, MELK, EBLN3P, ZCCHC7, RNF38

11. The ceDNA vector of claim 1, wherein the GSH locus is a region in any of the untranslated sequence or an intron or exon within any of the chromosomal regions selected from: chromosome 9 (36,833,275-37,034,185) (Pax6); Chromosome 6 (39,329,990-39,725,405) (Kif6) or Chromosome 16 (cdh 8: 61,647,242-62,036,835 cdh 11: 64,943,753-65,122,198).

12. The ceDNA vector of claim 1, wherein the GSH locus is a region in any of the untranslated sequence or an intron or exon of the genes selected from Accession numbers: NC_000009.12 (36833274 . . . 37035949, complement); NC_000009.12 (36864254 . . . 36864308, complement); NC_000009.12 (36823539 . . . 36823599, complement); NC_000009.12 (36893462 . . . 36893531, complement), NC_000009.12 (37046835 . . . 37047242); NC_000009.12 (37027763 . . . 37031333); NC_000009.12 (37002697 . . . 37007774); NC_000009.12 (36779475 . . . 36830456); NC_000009.12 (36572862 . . . 36677683); NC_000009.12 (37079896 . . . 37090401); NC_000009.12 (37120169 . . . 37358149) or NC_000009.12 (36336398 . . . 36487384, complement).

13. A capsid free, linear, closed-ended DNA (ceDNA) vector comprising two inverted terminal repeats (ITRs), and located between the two ITRs, a gene editing cassette, at least one heterologous nucleotide sequence, and at least one Genomic Safe Harbor Homology Arm (GSH HA),
wherein the gene editing cassette comprises at least one gene editing molecule selected from a nuclease, a guide RNA (gRNA), a guide DNA (gDNA), and an activator RNA, and
wherein the GSH HA binds to a target site located in a genomic safe harbor locus (GSH locus) in Table 1A or Table 1B, and wherein the GSH HA guides insertion of the heterologous nucleotide sequence into a locus located within the genomic safe harbor.

14. A capsid free, linear, closed-ended DNA (ceDNA) vector comprising two inverted terminal repeats (ITRs), and located between the two ITRs, at least one a guide RNA (gRNA) or at least one guide DNA (gDNA), and at least one heterologous nucleotide sequence, wherein the at least one gRNA or at least one gDNA binds to a target site located in a genomic safe harbor locus (GSH locus) in Table 1A or Table 1B, and wherein the gDNA or gRNA guides insertion of the heterologous nucleotide sequence into a locus located within the genomic safe harbor.

15. The ceDNA vector of claim 13 or 14, wherein the target site is in the PAX5 GSH locus or KIF6 GSH locus, and is a region of at least 100-1000 nucleotides located in Chromosome 9 (36,833,275-37,034,185 reverse strand), or Chromosome 6 (39,329,990-39,725,405).

16. The ceDNA vector of claim 13 or 14, wherein the GSH locus is a nucleic acid selected from any of the nucleic acid sequences listed in Table 1A or 1B.

17. The ceDNA vector of claim 13 or 14, wherein the GSH locus is a region in any of the untranslated sequence or an intron or exon of the genes selected from Kif6, KLHL7, NUPL2, mir684, KCNH2, GPNMB, MIR4540, MIR4475, MIR4476, PRL32P21, LOC105376031, LOC105376032, LOC105376030, MELK, EBLN3P, ZCCHC7, RNF38

18. The ceDNA vector of claim 13 or 14, wherein the GSH locus is a region in any of the untranslated sequence or an intron or exon within any of the chromosomal regions selected from: chromosome 9 (36,833,275-37,034,185) (Pax6); Chromosome 6 (39,329,990-39,725,405) (Kif6) or Chromosome 16 (cdh 8: 61,647,242-62,036,835 cdh 11: 64,943,753-65,122,198).

19. The ceDNA vector of claim 13 or 14, wherein the GSH locus is a region in any of the untranslated sequence or an intron or exon of the genes selected from Accession numbers: NC_000009.12 (36833274 . . . 37035949, complement); NC_000009.12 (36864254 . . . 36864308, complement); NC_000009.12 (36823539 . . . 36823599, complement); NC_000009.12 (36893462 . . . 36893531, complement), NC_000009.12 (37046835 . . . 37047242); NC_000009.12 (37027763 . . . 37031333); NC_000009.12 (37002697 . . . 37007774); NC_000009.12 (36779475 . . . 36830456); NC_000009.12 (36572862 . . . 36677683); NC_000009.12 (37079896 . . . 37090401); NC_000009.12 (37120169 . . . 37358149) or NC_000009.12 (36336398 . . . 36487384, complement).

20. The ceDNA vector of claim 13, wherein the ceDNA comprises at least a 5′ Genomic Safe Harbor Homology Arm (5′ GSH HA) or a 3′ Genomic Safe Harbor Homology Arm (3′ GSH HA), or both, wherein the 5′ GSH HA and the 3′ GSH HA bind to a target site located in a genomic safe harbor locus (GSH locus) in Table 1A or Table 1B, and wherein the 5′ GSH HA and/or the 3′ GSH HA guide insertion of the heterologous nucleotide sequence into a locus located within the genomic safe harbor.

21. The ceDNA vector of claim 20, wherein the heterologous nucleotide sequence is 3′ of the 5′ GSH HA, or 5′ of the 3′ GSH HA.

22. The ceDNA vector of claim 20, wherein the heterologous nucleotide sequence is located between the 5′ GSH HA and the 3′ GSH HA.

23. The ceDNA vector of claim 13 or 14, wherein insertion is by homologous recombination, homology direct repair (HDR), or non-homologous end joining (NHEJ).

24. The ceDNA vector of claim 13, wherein at least one gene editing molecule is a nuclease.

25. The ceDNA vector of claim 24, wherein the nuclease is a sequence specific nuclease or a nucleic acid-guided nuclease.

26. The ceDNA vector of claim 25, wherein the sequence specific nuclease is selected from a nucleic acid-guided nuclease, zinc finger nuclease (ZFN), a meganuclease, a transcription activator-like effector nuclease (TALEN), or a megaTAL.

27. The ceDNA vector of claim 26, wherein the sequence specific nuclease is a nucleic acid-guided nuclease selected from a single-base editor, an RNA-guided nuclease, and a DNA-guided nuclease.

28. The ceDNA vector of claim 13, wherein at least one gene editing molecule is a guide RNA (gRNA) or a guide DNA (gDNA), wherein the gRNA or gDNA binds to a region in the at least one GSH homology arm, or binds to a target site located in a genomic safe harbor locus (GSH locus) in Table 1A or Table 1B.

29. The ceDNA vector of claim 28, wherein the target site is in the PAX5 GSH locus, and is a region of at least 100-1000 nucleotides located in Chromosome 9 (36,833,275-37,034,185 reverse strand).

30. The ceDNA vector of claim 13, wherein at least one gene editing molecule is an activator RNA.

31. The ceDNA of any one of claim 25, wherein the nucleic acid-guided nuclease is a CRISPR nuclease.

32. The ceDNA vector of claim 31, wherein the CRISPR nuclease is a Cas nuclease.

33. The ceDNA vector of claim 32, wherein the Cas nuclease is selected from Cas9, nicking Cas9 (nCas9), and deactivated Cas (dCas).

34. The ceDNA vector of claim 33, wherein the nCas9 contains a mutation in the HNH or RuVc domain of Cas.

35. The ceDNA vector of claim 33, wherein the dCas is fused to a heterologous transcriptional activation domain that can be directed to a promoter region.

36. The ceDNA vector of any one of claims 33-36, wherein the dCas is S. pyogenes dCas9.

37. The ceDNA vector of any one of claim 14 or 28-36, wherein the guide RNA (gRNA) or guide DNA (gDNA) sequence binds to a region in the at least one GSH homology arm, or binds to a target site located in a genomic safe harbor locus (GSH locus) in Table 1A or Table 1B and CRISPR silences the target gene (CRISPRi system).

38. The ceDNA vector of any one of claim 14 or 28 or 37, wherein the guide RNA (gRNA) or guide DNA (gDNA) sequence targets a target site located in the 5′ GSH homology arm and activates insertion of the heterologous nucleic acid (CRISPRa system).

39. The ceDNA vector of any one of claim 13, 14 or 28, wherein the at least one gene editing molecule comprises a first guide RNA and a second guide RNA.

40. The ceDNA vector of claim 13, 14 or 28 or 39, wherein gDNA or gRNA effects non-homologous end joining (NHEJ) and insertion of the heterologous nucleic acid into a GSH locus.

41. The ceDNA vector of any one of claim 14 or 39, wherein the vector encodes multiple copies of one guide RNA sequence.

42. The ceDNA vector of claim 24, wherein a gene editing cassette comprises a first regulatory sequence operably linked to a nucleotide sequence that encodes a nuclease.

43. The ceDNA vector of claim 42, wherein the first regulatory sequence comprises a promoter.

44. The ceDNA vector of claim 43, wherein the promoter is CAG, Pol III, U6, or H1.

45. The ceDNA vector of any one of claims 42-44, wherein the first regulatory sequence comprises a modulator.

46. The ceDNA vector of claim 45, wherein the modulator is selected from an enhancer and a repressor.

47. The ceDNA vector of any one of claims 42-47, wherein the first heterologous nucleotide sequence comprises an intron sequence upstream of the nucleotide sequence that encodes the nuclease, wherein the intron sequence comprises a nuclease cleavage site.

48. The ceDNA vector of claim 42, wherein the gene editing cassette comprises a second heterologous nucleotide sequence comprises a second regulatory sequence operably linked to a nucleotide sequence that encodes a guide RNA (gRNA) or guide DNA (gDNA).

49. The ceDNA vector of claim 48, wherein the second regulatory sequence comprises a promoter.

50. The ceDNA vector of claim 49, wherein the promoter is CAG, Pol III, U6, or H1.

51. The ceDNA vector of any one of claims 48-50, wherein the second regulatory sequence comprises a modulator.

52. The ceDNA vector of claim 51, wherein the modulator is selected from an enhancer and a repressor.

53. The ceDNA vector of claim 48, wherein the gene editing cassette comprises a third heterologous nucleotide sequence comprising a third regulatory sequence operably linked to a nucleotide sequence that encodes an activator RNA.

54. The ceDNA vector of claim 53, wherein the third regulatory sequence comprises a promoter.

55. The ceDNA vector of claim 54, wherein the promoter is CAG, Pol III, U6, or H1.

56. The ceDNA vector of any one of claims 53-55, wherein the third regulatory sequence comprises a modulator.

57. The ceDNA vector of claim 56, wherein the modulator is selected from an enhancer and a repressor.

58. The ceDNA vector of any of claims 1-57, wherein the target site in the GSH locus is at least 1 kb in length.

59. The ceDNA vector of any of claims 1-57, wherein the target site in the GSH locus is between 300-3 kb in length.

60. The ceDNA vector of any of claims 1-57, wherein the target site in the GSH locus comprises a target site for a guide RNA (gRNA) or guide RNA (gRNA).

61. The ceDNA vector of any of claims 13, 14, 37, 48 and 60, wherein the gRNA or gDNA is for a sequence-specific nuclease selected from any of: a TAL-nuclease, a zinc-finger nuclease (ZFN), a meganuclease, a megaTAL, or an RNA guide endonuclease (e.g., CAS9, cpf1, nCAS9).

62. The ceDNA vector of any of claims 1-61, wherein at least one ITR comprises a functional terminal resolution site and a Rep binding site.

63. The ceDNA vector of any of claims 1-62, wherein the two ITRs are AAV ITRs.

64. The ceDNA vector of claim 63, wherein the AAV ITRs are AAV2 ITRs.

65. The ceDNA vector of any of claims 1-64, wherein the flanking ITRs are symmetric or asymmetric.

66. The ceDNA vector of any of claims 1-65, wherein the flanking ITRs are symmetrical or substantially symmetrical.

67. The ceDNA vector of any of claims 1-66, wherein the flanking ITRs are asymmetric.

68. The ceDNA vector of any of claims 1-67, wherein one or both of the ITRs are wild type, or wherein both of the ITRs are wild-type.

69. The ceDNA vector of any of claims 1-68, wherein the flanking ITRs are from different viral serotypes.

70. The ceDNA vector of any of claims 1-69, wherein one or both of the ITRs comprises a sequence selected from the sequences in Tables 6, 8A, 8B or 9.

71. The ceDNA vector of any of claims 1-70, wherein at least one of the ITRs is altered from a wild-type AAV ITR sequence by a deletion, addition, or substitution that affects the overall three-dimensional conformation of the ITR.

72. The ceDNA vector of any of claims 1-71, wherein one or both of the ITRs are derived from an AAV serotype selected from AAV1, AAV2, AAV3, AAV4, AAV5, AAV6, AAV7, AAV8, AAV9, AAV10, AAV11, and AAV12.

73. The ceDNA vector of any of claims 1-72, wherein one or both of the ITRs are synthetic.

74. The ceDNA vector of any of claims 1-73, wherein one or both of the ITRs is not a wild type ITR, or wherein both of the ITRs are not wild-type.

75. The ceDNA vector of any of claims 1-74, wherein one or both of the ITRs is modified by a deletion, insertion, and/or substitution in at least one of the ITR regions selected from A, A′, B, B′, C, C′, D, and D′.

76. The ceDNA vector of any of claims 1-75, wherein the deletion, insertion, and/or substitution results in the deletion of all or part of a stem-loop structure normally formed by the A, A′, B, B′ C, or C′ regions.

77. The ceDNA vector of any of claims 1-76, wherein one or both of the ITRs are modified by a deletion, insertion, and/or substitution that results in the deletion of all or part of a stem-loop structure normally formed by the B and B′ regions.

78. The ceDNA vector of any of claims 1-77, wherein one or both of the ITRs are modified by a deletion, insertion, and/or substitution that results in the deletion of all or part of a stem-loop structure normally formed by the C and C′ regions.

79. The ceDNA vector of any of claims 1-78, wherein one or both of the ITRs are modified by a deletion, insertion, and/or substitution that results in the deletion of part of a stem-loop structure normally formed by the B and B′ regions and/or part of a stem-loop structure normally formed by the C and C′ regions.

80. The ceDNA vector of any of claims 1-79, wherein one or both of the ITRs comprise a single stem-loop structure in the region that normally comprises a first stem-loop structure formed by the B and B′ regions and a second stem-loop structure formed by the C and C′ regions.

81. The ceDNA vector of any of claims 1-80, wherein one or both of the ITRs comprise a single stem and two loops in the region that normally comprises a first stem-loop structure formed by the B and B′ regions and a second stem-loop structure formed by the C and C′ regions.

82. The ceDNA vector of any of claims 1-82, wherein both ITRs are altered in a manner that results in an overall three-dimensional symmetry when the ITRs are inverted relative to each other.

83. The ceDNA vector of any of claims 1-82, wherein at least one heterologous nucleotide sequence is under the control of at least one regulatory switch or promoter.

84. The ceDNA vector of claim 83, wherein at least one regulatory switch is selected from a binary regulatory switch, a small molecule regulatory switch, a passcode regulatory switch, a nucleic acid-based regulatory switch, a post-transcriptional regulatory switch, a radiation-controlled or ultrasound controlled regulatory switch, a hypoxia-mediated regulatory switch, an inflammatory response regulatory switch, a shear-activated regulatory switch, and a kill switch.

85. The ceDNA vector of claim 84, wherein the promoter is an inducible promoter, or a tissue specific promoter or a constitutive promoter.

86. The ceDNA vector of any of claim 1-13 or 20-22, wherein the 5′ or 3′ GSH homology arms, or both are between 30-2000 bp in length.

87. The ceDNA vector of any of claims 1-86, wherein the heterologous nucleic acid comprises a transgene, and wherein the transgene is selected from any of: a nucleic acid, an inhibitor, peptide or polypeptide, antibody or antibody fragment, fusion protein, antigen, antagonist, agonist, RNAi molecule, miRNA, etc.

88. The ceDNA vector of any of claims 1-87, wherein heterologous nucleic acid sequence is in an orientation for integration into the genome at the GSH locus in a forward orientation.

89. The ceDNA vector of any of claims 1-88, wherein n heterologous nucleic acid sequence is in an orientation for integration into the genome at the GSH locus in a reverse orientation.

90. The ceDNA vector of any of claim 4, 13 or 20-22, wherein 5′ GSH homology arm and the 3′ GSH homology arm bind to target sites that are spatially distinct nucleic acid sequences in the genomic safe harbor locus disclosed in Tables 1A or 1B.

91. The ceDNA vector of any of claim 1-4, 13 or 20-22, wherein the at least one GSH-HA or GSH 5′ homology arm, or GSH 3′ homology arm are at least 65% complementary to a target sequence in the genomic safe harbor locus in Table 1A or Table 1B.

92. The ceDNA vector of any of claim 1-4, 13 or 20-22, wherein the at least one GSH-HA or 5′ GSH homology arm, or the GSH 3′ homology arm bind to a target site located in the PAX5 genomic safe harbor locus sequence.

93. The ceDNA vector of any of claim 1-4, 13 or 20-22, wherein the at least one GSH-HA, or 5′ GSH homology arm, or the GSH 3′ homology arm are at least 65% complementary to at least part the PAX5 genomic safe harbor locus sequence.

94. The ceDNA vector of any of claim 1-4, 13 or 20-22, wherein the at least GSH-HA, or 5′ GSH homology arm or the 3′ GSH homology arm bind to a target site located in a GSH locus located in a gene selected from Table 1A or 1B.

95. The ceDNA vector of any one of claims 1-94, comprising a first endonuclease restriction site upstream of the 5′ homology arm and/or a second endonuclease restriction site downstream of the 3′ homology arm.

96. The ceDNA vector of claim 95, wherein the first endonuclease restriction site and the second endonuclease restriction site are the same restriction endonuclease sites.

97. The ceDNA vector of claim 95-96, wherein at least one endonuclease restriction site is cleaved by a nuclease or endonuclease which is also encoded by a nucleic acid present in the gene editing cassette.

98. The ceDNA vector of any one of claims 1-97, wherein the heterologous nucleic acid or the gene editing cassette, or both, further comprises one or more poly-A sites.

99. The ceDNA vector of any one of claims 1-98, wherein the ceDNA vector comprises at least one of a regulatory element and a poly-A site 3′ of the 5′ GSH homology arm and/or 5′ of the 3′ GSH homology arm.

100. The ceDNA vector of any one of claims 1-99, where the heterologous nucleic acid further comprises a 2A and/or a nucleic acid encoding reporter protein 5′ of the 3′ GSH homology arm.

101. The ceDNA vector of any one of claim 13, 24 or 48-57, wherein the gene editing cassette further comprises a nucleic acid sequence encoding an enhancer of homologous recombination.

102. The ceDNA vector of claim 102, wherein the enhancer of homologous recombination is selected from SV40 late polyA signal upstream enhancer sequence, the cytomegalovirus early enhancer element, an RSV enhancer, and a CMV enhancer.

103. The ceDNA vector of any of claims 1-102, wherein the ceDNA vector is administered to a subject with a disease or disorder selected from cancer, autoimmune disease, a neurodegenerative disorder, hypercholesterolemia, acute organ rejection, multiple sclerosis, post-menopausal osteoporosis, skin conditions, asthma, or hemophilia.

104. The ceDNA vector of claim 103, wherein the cancer is selected from a solid tumor, soft tissue sarcoma, lymphoma, and leukemia.

105. The ceDNA vector of claim 103, wherein the autoimmune disease is selected from rheumatoid arthritis and Crohn's disease.

106. The ceDNA vector of claim 103, wherein the skin condition is selected from psoriasis and atopic dermatitis.

107. The ceDNA vector of claim 103, wherein the neurodegenerative disorder is Alzheimer's disease.

108. A cell comprising the ceDNA vector of any of claims 1-102.

109. The cell of claim 108, wherein the cell is a red blood cell (RBC) or RBC precursor cell.

110. The cell of claim 108, wherein the RBC precursor cell is a CD44+ or CD34+ cell.

111. The cell of claim 108, wherein the cell is a stem cell.

112. The cell of claim 108, wherein the cell is an iPS cell or embryonic stem cell.

113. The cell of claim 108, wherein the iPS cell is a patient-derived iPSC.

114. The cell of any of claims 108-113, wherein the cell is a mammalian cell.

115. The cell of claim 114, wherein the mammalian cell is a human cell.

116. The cell of claim 108, wherein the cell is ex vivo or in vivo, or in vitro.

117. The cell of claim 108, wherein the cell has been removed from a human subject.

118. The cell of claim 108, wherein the cell is present in a human or animal subject.

119. A kit comprising:
a. ceDNA vector composition of any of claims 1-102; and
i. at least one GSH 5′ primer and at least one GSH 3′ primer, wherein the GSH locus is any shown in Table 1A or 1B, wherein the at least one GSH 5′ primer binds to a region of the GSH locus upstream of the site of integration, and the at least one GSH 3′ primer is at least binds to a region of the GSH downstream of the site of integration; and/or
ii. at least two GSH 5′ primers comprising a forward GSH 5′ primer that binds to a region of the GSH upstream of the site of integration, and a reverse GSH 5′ primer that binds to a sequence in the nucleic acid inserted at the site of integration in the GSH sequence, wherein the GSH locus is any shown in Table 1A or 1B;
iii. at least two GSH 3′ primers comprising a forward GSH 3′ primer that binds to a sequence located at the 3′ end of the nucleic acid inserted at the site of integration in the GSH sequence, and a reverse GSH 3′ primer binds to a region of the GSH downstream of the site of integration, and wherein the GSH locus is any shown in Table 1A or 1B.

120. The kit of claim 119, wherein the ceDNA comprises at least one modified terminal repeat.

121. A kit comprising:
(a) a GSH-specific single guide and an RNA guided nucleic acid sequence present in one or more ceDNA vectors; and
(b) a ceDNA GSH knock-in vector comprising two inverted terminal repeats (ITRs), and located between the two ITRs, at least one heterologous nucleotide sequence located between a 5′ Genomic Safe Harbor Homology Arm (5′ GSH HA) and a 3′ Genomic Safe Harbor Homology Arm (3′ GSH HA), wherein the 5′ GSH HA and the 3′ GSH HA bind to a target site located in a genomic safe harbor locus (GSH locus) in Table 1A or Table 1B, and wherein the 5′ GSH HA and the 3′ GSH HA guide homologous recombination into a locus located within the genomic safe harbor,
wherein one or more of the sequences of (a) or (b) are comprised on a ceDNA vector of any of claims 1-1020.

122. The kit of claim 121, wherein the ceDNA GSH knock-in vector is a GSH-CRISPR-Cas vector.

123. The kit of claim 121, wherein the GSH CRISPR-Cas vector comprises a GSH-sgRNA nucleic acid sequence and Cas9 nucleic acid sequence.

124. The kit of claim 121, wherein the 5′ GSH homology arm and the 3′ GSH homology arm are at least 65% complementary to a sequence in the genomic safe harbor (GSH) of Table 1A or 1B, and wherein the GSH 5′ and 3′ homology arms guide insertion by homologous recombination, of the nucleic acid sequence located between the GSH 5′ homology arm and a GSH 3′ homology arm into a GSH locus located within the genomic safe harbor of one in Table 1A or 1B.

125. The kit of claim 121, wherein the GSH knockin donor vector is a PAX5 knockin donor vector comprising a PAX5 5′ homology arm and a PAX5 3′ homology arm, wherein the PAX5 5′ homology arm and the PAX5 3′ homology arm are at least 65% complementary to the PAX5 genomic safe harbor locus, and wherein the PAX5 5′ and 3′ homology arms guide insertion, by homologous recombination, of the nucleic acid located between the GSH 5′ homology arm and a GSH 3′ homology arm into a locus within the PAX5 genomic safe harbor.

126. The kit of claim 121, wherein the GSH knockin donor vector is a knockin donor vector comprising a 5′ homology arm which binds to a GSH locus listed in Table 1A or 1B, and a 3′ homology arm which binds to a spatially distinct region of the same GSH locus that the 5′ homology arm binds to, wherein the 5′ and 3′ homology arms guide insertion, by homologous recombination, of the nucleic acid located between the GSH 5′ homology arm and a GSH 3′ homology arm into a GSH locus listed in Table 1A or 1B.

127. The kit of any of claim 121, further comprising at least one GSH 5′ primer and at least one GSH 3′ primer, wherein the GSH is identified by the ceDNA vector of any of claims 41 to 51, wherein the at least one GSH 5′ primer is at least 80% complementary to a region of the GSH upstream of the site of integration, and the at least one GSH 3′ primer is at least 80% complementary to a region of the GSH downstream of the site of integration.

128. The kit of any of claims 121-127, further comprising at least two GSH 5′ primers comprising;
a. a forward GSH 5′ primer that is at least 80% complementary to a region of the GSH upstream of the site of integration, and
b. a reverse GSH 5′ primer that is at least 80% complementary to a sequence in the nucleic acid inserted at the site of integration in the GSH sequence,
wherein the GSH is identified by the ceDNA vector of any of claims 41 to 51.

129. The kit of any of claims 121-128, further comprising at least two GSH 3′ primers comprising;
a. a forward GSH 3′ primer that is at least 80% complementary to a sequence located at the 3′ end of the nucleic acid inserted at the site of integration in the GSH sequence, and
b. a reverse GSH 3′ primer that is at least 80% complementary to a region of the GSH downstream of the site of integration, and
wherein the GSH is identified by the ceDNA vector of any of claims 41 to 51.

130. The kit of any of claims 121-129, wherein the GSH 5′ primer is a PAX5 5′ primer and the GSH 3′ primer is a PAX 3′ primer, wherein the PAX5 5′ primer and the PAX5 3′ primer flank the site of integration in the PAX5 genomic safe harbor.

131. A method of generating a genetically modified animal comprising a nucleic acid interest inserted at a PAX5 Genomic Safe Harbor (GSH) locus, comprising a) introducing into a host cell a ceDNA of any of claims 1-102, and b) introducing the cell generated in (a) into a carrier animal to produce a genetically modified animal.

132. The ceDNA vector of claim 131, wherein the host cell is a zygote or a pluripotent stem cell.

133. A genetically modified animal produced by the ceDNA vector of claim 131.


SEQUENCE LISTING (190 sequences)

SEQ ID NO: 1 - DNA, 141 bp, Artificial Sequence
  Description of Artificial Sequence Synthetic polynucleotide
aggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcagg

SEQ ID NO: 2 - DNA, 141 bp, Artificial Sequence
  Description of Artificial Sequence Synthetic polynucleotide
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcct

SEQ ID NO: 3 - DNA, 130 bp, Artificial Sequence
  Description of Artificial Sequence Synthetic polynucleotide
aggaacccctagtgatggagttggccactccctctctgcgcgctcgctcgctcactgaggccgggcgaccaaaggtcgcccgacgcccgggcggcctcagtgagcgagcgagcgcgcagctgcctgcagg

SEQ ID NO: 4 - DNA, 130 bp, Artificial Sequence
  Description of Artificial Sequence Synthetic polynucleotide
cctgcaggcagctgcgcgctcgctcgctcactgaggccgcccgggcgtcgggcgacctttggtcgcccggcctcagtgagcgagcgagcgcgcagagagggagtggccaactccatcactaggggttcct

SEQ ID NO: 5 - DNA, 143 bp, Artificial Sequence
  Description of Artificial Sequence Synthetic polynucleotide
ttgcccactccctctctgcgcgctcgctcgctcggtggggcctgcggaccaaaggtccgcagacggcagaggtctcctctgccggccccaccgagcgagcgacgcgcgcagagagggagtgggcaactccatcactagggtaa

SEQ ID NO: 6 - DNA, 144 bp, Artificial Sequence
  Description of Artificial Sequence Synthetic polynucleotide
ttggccactccctctatgcgcactcgctcgctcggtggggcctggcgaccaaaggtcgccagacggacgtgggtttccacgtccggccccaccgagcgagcgagtgcgcatagagggagtggccaactccatcactagaggtat

SEQ ID NO: 7 - DNA, 127 bp, Artificial Sequence
  Description of Artificial Sequence Synthetic polynucleotide
ttggccactccctctatgcgcgctcgctcactcactcggccctggagaccaaaggtctccagactgccggcctctggccggcagggccgagtgagtgagcgagcgcgcatagagggagtggccaact

SEQ ID NO: 8 - DNA, 166 bp, Artificial Sequence
  Description of Artificial Sequence Synthetic polynucleotide
tcccccctgtcgcgttcgctcgctcgctggctcgtttgggggggcgacggccagagggccgtcgtctggcagctctttgagctgccacccccccaaacgagccagcgagcgagcgaacgcgacaggggggagagtgccacactctcaagcaagggggttttgtaag

SEQ ID NO: 9 - DNA, 144 bp, Artificial Sequence
  Description of Artificial Sequence Synthetic polynucleotide
ttgcccactccctctaatgcgcgctcgctcgctcggtggggcctgcggaccaaaggtccgcagacggcagaggtctcctctgccggccccaccgagcgagcgagcgcgcatagagggagtgggcaactccatcactaggggtat

SEQ ID NO: 10 - DNA, 143 bp, Artificial Sequence
  Description of Artificial Sequence Synthetic polynucleotide
ttaccctagtgatggagttgcccactccctctctgcgcgcgtcgctcgctcggtggggccggcagaggagacctctgccgtctgcggacctttggtccgcaggccccaccgagcgagcgagcgcgcagagagggagtgggcaa

SEQ ID NO: 11 - DNA, 144 bp, Artificial Sequence
  Description of Artificial Sequence Synthetic polynucleotide
atacctctagtgatggagttggccactccctctatgcgcactcgctcgctcggtggggccggacgtggaaacccacgtccgtctggcgacctttggtcgccaggccccaccgagcgagcgagtgcgcatagagggagtggccaa

SEQ ID NO: 12 - DNA, 127 bp, Artificial Sequence
  Description of Artificial Sequence Synthetic polynucleotide
agttggccacattagctatgcgcgctcgctcactcactcggccctggagaccaaaggtctccagactgccggcctctggccggcagggccgagtgagtgagcgagcgcgcatagagggagtggccaa

SEQ ID NO: 13 - DNA, 166 bp, Artificial Sequence
  Description of Artificial Sequence Synthetic polynucleotide
cttacaaaacccccttgcttgagagtgtggcactctcccccctgtcgcgttcgctcgctcgctggctcgtttgggggggtggcagctcaaagagctgccagacgacggccctctggccgtcgcccccccaaacgagccagcgagcgagcgaacgcgacagggggga

SEQ ID NO: 14 - DNA, 144 bp, Artificial Sequence
  Description of Artificial Sequence Synthetic polynucleotide
atacccctagtgatggagttgcccactccctctatgcgcgctcgctcgctcggtggggccggcagaggagacctctgccgtctgcggacctttggtccgcaggccccaccgagcgagcgagcgcgcattagagggagtgggcaa

SEQ ID NO: 15 - DNA, 120 bp, Artificial Sequence
  Description of Artificial Sequence Synthetic polynucleotide
aggaacccctagtgatggagttggccactccctctctgcgcgctcgctcgctcactgaggcgcacgcccgggtttcccgggcggcctcagtgagcgagcgagcgcgcagctgcctgcagg

SEQ ID NO: 16 - DNA, 122 bp, Artificial Sequence
  Description of Artificial Sequence Synthetic polynucleotide
aggaacccctagtgatggagttggccactccctctctgcgcgctcgctcgctcactgaggccgacgcccgggctttgcccgggcggcctcagtgagcgagcgagcgcgcagctgcctgcagg

SEQ ID NO: 17 - DNA, 129 bp, Artificial Sequence
  Description of Artificial Sequence Synthetic polynucleotide
aggaacccctagtgatggagttggccactccctctctgcgcgctcgctcgctcactgaggccgggcgaccaaaggtcgcccgacgcccgggcgcctcagtgagcgagcgagcgcgcagctgcctgcagg

SEQ ID NO: 18 - DNA, 101 bp, Artificial Sequence
  Description of Artificial Sequence Synthetic polynucleotide
aggaacccctagtgatggagttggccactccctctctgcgcgctcgctcgctcactgaggctttgcctcagtgagcgagcgagcgcgcagctgcctgcagg

SEQ ID NO: 19 - DNA, 139 bp, Artificial Sequence
  Description of Artificial Sequence Synthetic polynucleotide
aggaacccctagtgatggagttggccactccctctctgcgcgctcgctcgctcactgaggccgggcgacaaagtcgcccgacgcccgggctttgcccgggcggcctcagtgagcgagcgagcgcgcagctgcctgcagg

SEQ ID NO: 20 - DNA, 137 bp, Artificial Sequence
  Description of Artificial Sequence Synthetic polynucleotide
aggaacccctagtgatggagttggccactccctctctgcgcgctcgctcgctcactgaggccgggcgaaaatcgcccgacgcccgggctttgcccgggcggcctcagtgagcgagcgagcgcgcagctgcctgcagg

SEQ ID NO: 21 - DNA, 135 bp, Artificial Sequence
  Description of Artificial Sequence Synthetic polynucleotide
aggaacccctagtgatggagttggccactccctctctgcgcgctcgctcgctcactgaggccgggcgaaacgcccgacgcccgggctttgcccgggcggcctcagtgagcgagcgagcgcgcagctgcctgcagg

SEQ ID NO: 22 - DNA, 133 bp, Artificial Sequence
  Description of Artificial Sequence Synthetic polynucleotide
aggaacccctagtgatggagttggccactccctctctgcgcgctcgctcgctcactgaggccgggcaaagcccgacgcccgggctttgcccgggcggcctcagtgagcgagcgagcgcgcagctgcctgcagg

SEQ ID NO: 23 - DNA, 139 bp, Artificial Sequence
  Description of Artificial Sequence Synthetic polynucleotide
aggaacccctagtgatggagttggccactccctctctgcgcgctcgctcgctcactgaggccgggcgaccaaaggtcgcccgacgcccgggtttcccgggcggcctcagtgagcgagcgagcgcgcagctgcctgcagg

SEQ ID NO: 24 - DNA, 137 bp, Artificial Sequence
  Description of Artificial Sequence Synthetic polynucleotide
aggaacccctagtgatggagttggccactccctctctgcgcgctcgctcgctcactgaggccgggcgaccaaaggtcgcccgacgcccggtttccgggcggcctcagtgagcgagcgagcgcgcagctgcctgcagg

SEQ ID NO: 25 - DNA, 135 bp, Artificial Sequence
  Description of Artificial Sequence Synthetic polynucleotide
aggaacccctagtgatggagttggccactccctctctgcgcgctcgctcgctcactgaggccgggcgaccaaaggtcgcccgacgcccgtttcgggcggcctcagtgagcgagcgagcgcgcagctgcctgcagg

SEQ ID NO: 26 - DNA, 133 bp, Artificial Sequence
  Description of Artificial Sequence Synthetic polynucleotide
aggaacccctagtgatggagttggccactccctctctgcgcgctcgctcgctcactgaggccgggcgaccaaaggtcgcccgacgccctttgggcggcctcagtgagcgagcgagcgcgcagctgcctgcagg

SEQ ID NO: 27 - DNA, 131 bp, Artificial Sequence
  Description of Artificial Sequence Synthetic polynucleotide
aggaacccctagtgatggagttggccactccctctctgcgcgctcgctcgctcactgaggccgggcgaccaaaggtcgcccgacgcctttggcggcctcagtgagcgagcgagcgcgcagctgcctgcagg

SEQ ID NO: 28 - DNA, 129 bp, Artificial Sequence
  Description of Artificial Sequence Synthetic polynucleotide
aggaacccctagtgatggagttggccactccctctctgcgcgctcgctcgctcactgaggccgggcgaccaaaggtcgcccgacgctttgcggcctcagtgagcgagcgagcgcgcagctgcctgcagg

SEQ ID NO: 29 - DNA, 127 bp, Artificial Sequence
  Description of Artificial Sequence Synthetic polynucleotide
aggaacccctagtgatggagttggccactccctctctgcgcgctcgctcgctcactgaggccgggcgaccaaaggtcgcccgacgtttcggcctcagtgagcgagcgagcgcgcagctgcctgcagg

SEQ ID NO: 30 - DNA, 122 bp, Artificial Sequence
  Description of Artificial Sequence Synthetic polynucleotide
aggaacccctagtgatggagttggccactccctctctgcgcgctcgctcgctcactgaggccgggcgaccaaaggtcgcccgacggcctcagtgagcgagcgagcgcgcagctgcctgcagg

SEQ ID NO: 31 - DNA, 130 bp, Artificial Sequence; identical to SEQ ID NO: 3
  Description of Artificial Sequence Synthetic polynucleotide

SEQ ID NO: 32 - DNA, 120 bp, Artificial Sequence
  Description of Artificial Sequence Synthetic polynucleotide
cctgcaggcagctgcgcgctcgctcgctcactgaggccgcccgggaaacccgggcgtgcgcctcagtgagcgagcgagcgcgcagagagggagtggccaactccatcactaggggttcct

SEQ ID NO: 33 - DNA, 122 bp, Artificial Sequence
  Description of Artificial Sequence Synthetic polynucleotide
cctgcaggcagctgcgcgctcgctcgctcactgaggccgtcgggcgacctttggtcgcccggcctcagtgagcgagcgagcgcgcagagagggagtggccaactccatcactaggggttcct

SEQ ID NO: 34 - DNA, 122 bp, Artificial Sequence
  Description of Artificial Sequence Synthetic polynucleotide
cctgcaggcagctgcgcgctcgctcgctcactgaggccgcccgggcaaagcccgggcgtcggcctcagtgagcgagcgagcgcgcagagagggagtggccaactccatcactaggggttcct

SEQ ID NO: 35 - DNA, 129 bp, Artificial Sequence
  Description of Artificial Sequence Synthetic polynucleotide
cctgcaggcagctgcgcgctcgctcgctcactgaggcgcccgggcgtcgggcgacctttggtcgcccggcctcagtgagcgagcgagcgcgcagagagggagtggccaactccatcactaggggttcct

SEQ ID NO: 36 - DNA, 101 bp, Artificial Sequence
  Description of Artificial Sequence Synthetic polynucleotide
cctgcaggcagctgcgcgctcgctcgctcactgaggcaaagcctcagtgagcgagcgagcgcgcagagagggagtggccaactccatcactaggggttcct

SEQ ID NO: 37 - DNA, 139 bp, Artificial Sequence
  Description of Artificial Sequence Synthetic polynucleotide
cctgcaggcagctgcgcgctcgctcgctcactgaggccgcccgggcaaagcccgggcgtcgggcgactttgtcgcccggcctcagtgagcgagcgagcgcgcagagagggagtggccaactccatcactaggggttcct

SEQ ID NO: 38 - DNA, 137 bp, Artificial Sequence
  Description of Artificial Sequence Synthetic polynucleotide
cctgcaggcagctgcgcgctcgctcgctcactgaggccgcccgggcaaagcccgggcgtcgggcgattttcgcccggcctcagtgagcgagcgagcgcgcagagagggagtggccaactccatcactaggggttcct

SEQ ID NO: 39 - DNA, 135 bp, Artificial Sequence
  Description of Artificial Sequence Synthetic polynucleotide
cctgcaggcagctgcgcgctcgctcgctcactgaggccgcccgggcaaagcccgggcgtcgggcgtttcgcccggcctcagtgagcgagcgagcgcgcagagagggagtggccaactccatcactaggggttcct

SEQ ID NO: 40 - DNA, 133 bp, Artificial Sequence
  Description of Artificial Sequence Synthetic polynucleotide
cctgcaggcagctgcgcgctcgctcgctcactgaggccgcccgggcaaagcccgggcgtcgggctttgcccggcctcagtgagcgagcgagcgcgcagagagggagtggccaactccatcactaggggttcct

SEQ ID NO: 41 - DNA, 139 bp, Artificial Sequence
  Description of Artificial Sequence Synthetic polynucleotide
cctgcaggcagctgcgcgctcgctcgctcactgaggccgcccgggaaacccgggcgtcgggcgacctttggtcgcccggcctcagtgagcgagcgagcgcgcagagagggagtggccaactccatcactaggggttcct

SEQ ID NO: 42 - DNA, 137 bp, Artificial Sequence
  Description of Artificial Sequence Synthetic polynucleotide
cctgcaggcagctgcgcgctcgctcgctcactgaggccgcccggaaaccgggcgtcgggcgacctttggtcgcccggcctcagtgagcgagcgagcgcgcagagagggagtggccaactccatcactaggggttcct

SEQ ID NO: 43 - DNA, 135 bp, Artificial Sequence
  Description of Artificial Sequence Synthetic polynucleotide
cctgcaggcagctgcgcgctcgctcgctcactgaggccgcccgaaacgggcgtcgggcgacctttggtcgcccggcctcagtgagcgagcgagcgcgcagagagggagtggccaactccatcactaggggttcct

SEQ ID NO: 44 - DNA, 133 bp, Artificial Sequence
  Description of Artificial Sequence Synthetic polynucleotide
cctgcaggcagctgcgcgctcgctcgctcactgaggccgcccaaagggcgtcgggcgacctttggtcgcccggcctcagtgagcgagcgagcgcgcagagagggagtggccaactccatcactaggggttcct

SEQ ID NO: 45 - DNA, 131 bp, Artificial Sequence
  Description of Artificial Sequence Synthetic polynucleotide
cctgcaggcagctgcgcgctcgctcgctcactgaggccgccaaaggcgtcgggcgacctttggtcgcccggcctcagtgagcgagcgagcgcgcagagagggagtggccaactccatcactaggggttcct

SEQ ID NO: 46 - DNA, 129 bp, Artificial Sequence
  Description of Artificial Sequence Synthetic polynucleotide
cctgcaggcagctgcgcgctcgctcgctcactgaggccgcaaagcgtcgggcgacctttggtcgcccggcctcagtgagcgagcgagcgcgcagagagggagtggccaactccatcactaggggttcct

SEQ ID NO: 47 - DNA, 127 bp, Artificial Sequence
  Description of Artificial Sequence Synthetic polynucleotide
cctgcaggcagctgcgcgctcgctcgctcactgaggccgaaacgtcgggcgacctttggtcgcccggcctcagtgagcgagcgagcgcgcagagagggagtggccaactccatcactaggggttcct

SEQ ID NO: 48 - DNA, 122 bp, Artificial Sequence; identical to SEQ ID NO: 30
  Description of Artificial Sequence Synthetic polynucleotide

SEQ ID NO: 49 - DNA, 12 bp, Artificial Sequence
  Description of Artificial Sequence Synthetic oligonucleotide
cgatcgttcgat

SEQ ID NO: 50 - DNA, 12 bp, Artificial Sequence
  Description of Artificial Sequence Synthetic oligonucleotide
atcgaaccatcg

SEQ ID NO: 51 - DNA, 12 bp, Artificial Sequence
  Description of Artificial Sequence Synthetic oligonucleotide
atcgaacgatcg

SEQ ID NO: 52 - DNA, 165 bp, Artificial Sequence
  Description of Artificial Sequence Synthetic polynucleotide
aggaacccctagtgatggagttggccactccctctctgcgcgctcgctcgctcactgaggccgcccgggcaaagcccgggcgtcgggcgacctttggtcgcccggcctcagtgagcgagcgagcgcgcagagagggagtggccaactccatcactaggggttcct

SEQ ID NO: 53 - DNA, 140 bp, Artificial Sequence
  Description of Artificial Sequence Synthetic polynucleotide
cccctagtgatggagttggccactccctctctgcgcgctcgctcgctcactgaggccgcccgggcaaagcccgggcgtcgggcgacctttggtcgcccggcctcagtgagcgagcgagcgcgcagagagatcactagggg

SEQ ID NO: 54 - DNA, 91 bp, Artificial Sequence
  Description of Artificial Sequence Synthetic oligonucleotide
gcgcgctcgctcgctcactgaggccgcccgggcaaagcccgggcgtcgggcgacctttggtcgcccggcctcagtgagcgagcgagcgcgc

SEQ ID NO: 55 - DNA, 91 bp, Artificial Sequence
  Description of Artificial Sequence Synthetic oligonucleotide
gcgcgctcgctcgctcactgaggccgggcgaccaaaggtcgcccgacgcccgggctttgcccgggcggcctcagtgagcgagcgagcgcgc

SEQ ID NO: 56 - DNA, 1662 bp, Artificial Sequence
  Description of Artificial Sequence Synthetic polynucleotide
gccgccaccatggaagacgccaaaaacataaagaaaggcccggcgccattctatccgctggaagatggaaccgctggagagcaactgcataaggctatgaagagatacgccctggttcctggaacaattgcttttacagatgcacatatcgaggtggacatcacttacgctgagtacttcgaaatgtccgttcggttggcagaagctatgaaacgatatgggctgaatacaaatcacagaatcgtcgtatgcagtgaaaactctcttcaattctttatgccggtgttgggcgcgttatttatcggagttgcagttgcgcccgcgaacgacatttataatgaacgtgaattgctcaacagtatgggcatttcgcagcctaccgtggtgttcgtttccaaaaaggggttgcaaaaaattttgaacgtgcaaaaaaagctcccaatcatccaaaaaattattatcatggattctaaaacggattaccagggatttcagtcgatgtacacgttcgtcacatctcatctacctcccggttttaatgaatacgattttgtgccagagtccttcgatagggacaagacaattgcactgatcatgaactcctctggatctactggtctgcctaaaggtgtcgctctgcctcatagaactgcctgcgtgagattctcgcatgccagagatcctatttttggcaatcaaatcattccggatactgcgattttaagtgttgttccattccatcacggttttggaatgtttactacactcggatatttgatatgtggatttcgagtcgtcttaatgtatagatttgaagaagagctgtttctgaggagccttcaggattacaagattcaaagtgcgctgctggtgccaaccctattctccttcttcgccaaaagcactctgattgacaaatacgatttatctaatttacacgaaattgcttctggtggcgctcccctctctaaggaagtcggggaagcggttgccaagaggttccatctgccaggtatcaggcaaggatatgggctcactgagactacatcagctattctgattacacccgagggggatgataaaccgggcgcggtcggtaaagttgttccattttttgaagcgaaggttgtggatctggataccgggaaaacgctgggcgttaatcaaagaggcgaactgtgtgtgagaggtcctatgattatgtccggttatgtaaacaatccggaagcgaccaacgccttgattgacaaggatggatggctacattctggagacatagcttactgggacgaagacgaacacttcttcatcgttgaccgcctgaagtctctgattaagtacaaaggctatcaggtggctcccgctgaattggaatccatcttgctccaacaccccaacatcttcgacgcaggtgtcgcaggtcttcccgacgatgacgccggtgaacttcccgccgccgttgttgttttggagcacggaaagacgatgacggaaaaagagatcgtggattacgtcgccagtcaagtaacaaccgcgaaaaagttgcgcggaggagttgtgtttgtggacgaagtaccgaaaggtcttaccggaaaactcgacgcaagaaaaatcagagagatcctcataaaggccaagaagggcggaaagatcgccgtgtaa

SEQ ID NO: 57 - protein, 453 aa, Artificial Sequence
  Description of Artificial Sequence Synthetic polypeptide
  MOD_RES (1)..(1): Any amino acid
XVQLVESGGGVVQPGRSLRLSCAASGFAFSSYGMHWVRQAPGKGLEWVAVIWFDGTKKYYTDSVKGRFTISRDNSKNTLYLQMNTLRAEDTAVYYCARDRGIGARRGPYYMDVWGKGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 58 - protein, 214 aa, Artificial Sequence
  Description of Artificial Sequence Synthetic polypeptide
DIQMTQSPSSLSASVGDRVTITCRASQSISSYLNWYQQKPGKAPKLLIYAASSLQSGVPSRFSGSGSGTDFTLTISSLQPEDFATYYCQQSYSTPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 59 - DNA, 1310 bp, Artificial Sequence
  Description of Artificial Sequence Synthetic polynucleotide
ggagccgagagtaattcatacaaaaggagggatcgccttcgcaaggggagagcccagggaccgtccctaaattctcacagacccaaatccctgtagccgccccacgacagcgcgaggagcatgcgctcagggctgagcgcggggagagcagagcacacaagctcatagaccctggtcgtgggggggaggaccggggagctggcgcggggcaaactgggaaagcggtgtcgtgtgctggctccgccctcttcccgagggtgggggagaacggtatataagtgcggcagtcgccttggacgttctttttcgcaacgggtttgccgtcagaacgcaggtgaggggcgggtgtggcttccgcgggccgccgagctggaggtcctgctccgagcgggccgggccccgctgtcgtcggcggggattagctgcgagcattcccgcttcgagttgcgggcggcgcgggaggcagagtgcgaggcctagcggcaaccccgtagcctcgcctcgtgtccggcttgaggcctagcgtggtgtccgcgccgccgccgcgtgctactccggccgcactctggtcttttttttttttgttgttgttgccctgctgccttcgattgccgttcagcaataggggctaacaaagggagggtgcggggcttgctcgcccggagcccggagaggtcatggttggggaggaatggagggacaggagtggcggctggggcccgcccgccttcggagcacatgtccgacgccacctggatggggcgaggcctggggtttttcccgaagcaaccaggctggggttagcgtgccgaggccatgtggccccagcacccggcacgatctggcttggcggcgccgcgttgccctgcctccctaactagggtgaggccatcccgtccggcaccagttgcgtgcgtggaaagatggccgctcccgggccctgttgcaaggagctcaaaatggaggacgcggcagcccggtggagcgggcgggtgagtcacccacacaaaggaagagggcctggtccctcaccggctgctgcttcctgtgaccccgtggtcctatcggccgcaatagtcacctcgggcttttgagcacggctagtcgcggcggggggaggggatgtaatggcgttggagtttgttcacatttggtgggtggagactagtcaggccagcctggcgctggaagtcatttttggaatttgtccccttgagttttgagcggagctaattctcgggcttcttagcggttcaaaggtatcttttaaacccttttttaggtgttgtgaaaaccaccgctaattcaaa

SEQ ID NO: 60 - DNA, 16 bp, Artificial Sequence
  Description of Artificial Sequence Synthetic oligonucleotide
gcgcgctcgctcgctc

SEQ ID NO: 61 - DNA, 6 bp, Artificial Sequence
  Description of Artificial Sequence Synthetic oligonucleotide
ggttga

SEQ ID NO: 62 - DNA, 4 bp, Artificial Sequence
  Description of Artificial Sequence Synthetic oligonucleotide
agtt

SEQ ID NO: 63 - DNA, 6 bp, Artificial Sequence
  Description of Artificial Sequence Synthetic oligonucleotide
ggttgg

SEQ ID NO: 64 - DNA, 6 bp, Artificial Sequence
  Description of Artificial Sequence Synthetic oligonucleotide
agttgg

SEQ ID NO: 65 - DNA, 6 bp, Artificial Sequence
  Description of Artificial Sequence Synthetic oligonucleotide
agttga

SEQ ID NO: 66 - DNA, 6 bp, Artificial Sequence
  Description of Artificial Sequence Synthetic oligonucleotide
rrttrr

SEQ ID NO: 67 - DNA, 581 bp, Artificial Sequence
  Description of Artificial Sequence Synthetic polynucleotide
gagcatcttaccgccatttattcccatatttgttctgtttttcttgatttgggtatacatttaaatgttaataaaacaaaatggtggggcaatcatttacatttttagggatatgtaattactagttcaggtgtattgccacaagacaaacatgttaagaaactttcccgttatttacgctctgttcctgttaatcaacctctggattacaaaatttgtgaaagattgactgatattcttaactatgttgctccttttacgctgtgtggatatgctgctttatagcctctgtatctagctattgcttcccgtacggctttcgttttctcctccttgtataaatcctggttgctgtctcttttagaggagttgtggcccgttgtccgtcaacgtggcgtggtgtgctctgtgtttgctgacgcaacccccactggctggggcattgccaccacctgtcaactcctttctgggactttcgctttccccctcccgatcgccacggcagaactcatcgccgcctgccttgcccgctgctggacaggggctaggttgctgggcactgataattccgtggtgttgtc

SEQ ID NO: 68 - DNA, 225 bp, Artificial Sequence
  Description of Artificial Sequence Synthetic polynucleotide
tgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggc

SEQ ID NO: 69 - DNA, 8 bp, Artificial Sequence
  Description of Artificial Sequence Synthetic oligonucleotide
actgaggc

SEQ ID NO: 70 - DNA, 8 bp, Artificial Sequence
  Description of Artificial Sequence Synthetic oligonucleotide
gcctcagt

SEQ ID NO: 71 - DNA, 16 bp, Artificial Sequence
  Description of Artificial Sequence Synthetic oligonucleotide
gagcgagcgagcgcgc

SEQ ID NO: 72 - DNA, 1923 bp, Artificial Sequence
  Description of Artificial Sequence Synthetic polynucleotide
tcaatattggccattagccatattattcattggttatatagcataaatcaatattggctattggccattgcatacgttgtatctatatcataatatgtacatttatattggctcatgtccaatatgaccgccatgttggcattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtccgccccctattgacgtcaatgacggtaaatggcccgcctggcattatgcccagtacatgaccttacgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgacgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcgggggggagcggctcggggggtgcgtgcgtgtgtgtgtgcgtggggagcgccgcgtgcggcccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcggcggtcgggctgtaacccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttttagccttcttctttttcctacagctcctgggcaacgtgctggttattgtgctgtctcatcatttgtcgacagaattcctcgaagatccgaaggggttcaagcttggcattccggtactgttggtaaagcca

SEQ ID NO: 73 - DNA, 1272 bp, Artificial Sequence
  Description of Artificial Sequence Synthetic polynucleotide
aggctcagaggcacacaggagtttctgggctcaccctgcccccttccaacccctcagttcccatcctccagcagctgtttgtgtgctgcctctgaagtccacactgaacaaacttcagcctactcatgtccctaaaatgggcaaacattgcaagcagcaaacagcaaacacacagccctccctgcctgctgaccttggagctggggcagaggtcagagacctctctgggcccatgccacctccaacatccactcgaccccttggaatttcggtggagaggagcagaggttgtcctggcgtggtttaggtagtgtgagagggtccgggttcaaaaccacttgctgggtggggagtcgtcagtaagtggctatgccccgaccccgaagcctgtttccccatctgtacaatggaaatgataaagacgcccatctgatagggtttttgtggcaaataaacatttggtttttttgttttgttttgttttgttttttgagatggaggtttgctctgtcgcccaggctggagtgcagtgacacaatctcatctcaccacaaccttcccctgcctcagcctcccaagtagctgggattacaagcatgtgccaccacacctggctaattttctatttttagtagagacgggtttctccatgttggtcagcctcagcctcccaagtaactgggattacaggcctgtgccaccacacccggctaattttttctatttttgacagggacggggtttcaccatgttggtcaggctggtctagaggtaccggatcttgctaccagtggaacagccactaaggattctgcagtgagagcagagggccagctaagtggtactctcccagagactgtctgactcacgccaccccctccaccttggacacaggacgctgtggtttctgagccaggtacaatgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagtgaatccggactctaaggtaaatataaaatttttaagtgtataatgtgttaaactactgattctaattgtttctctcttttagattccaacctttggaactga

SEQ ID NO: 74 - DNA, 1177 bp, Artificial Sequence
  Description of Artificial Sequence Synthetic polynucleotide
ggctcagaggctcagaggcacacaggagtttctgggctcaccctgcccccttccaacccctcagttcccatcctccagcagctgtttgtgtgctgcctctgaagtccacactgaacaaacttcagcctactcatgtccctaaaatgggcaaacattgcaagcagcaaacagcaaacacacagccctccctgcctgctgaccttggagctggggcagaggtcagagacctctctgggcccatgccacctccaacatccactcgaccccttggaatttcggtggagaggagcagaggttgtcctggcgtggtttaggtagtgtgagagggtccgggttcaaaaccacttgctgggtggggagtcgtcagtaagtggctatgccccgaccccgaagcctgtttccccatctgtacaatggaaatgataaagacgcccatctgatagggtttttgtggcaaataaacatttggtttttttgttttgttttgttttgttttttgagatggaggtttgctctgtcgcccaggctggagtgcagtgacacaatctcatctcaccacaaccttcccctgcctcagcctcccaagtagctgggattacaagcatgtgccaccacacctggctaattttctatttttagtagagacgggtttctccatgttggtcagcctcagcctcccaagtaactgggattacaggcctgtgccaccacacccggctaattttttctatttttgacagggacggggtttcaccatgttggtcaggctggtctagaggtaccggatcttgctaccagtggaacagccactaaggattctgcagtgagagcagagggccagctaagtggtactctcccagagactgtctgactcacgccaccccctccaccttggacacaggacgctgtggtttctgagccaggtacaatgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagt

SEQ ID NO: 75 - DNA, 547 bp, Artificial Sequence
  Description of Artificial Sequence Synthetic polynucleotide
ccctaaaatgggcaaacattgcaagcagcaaacagcaaacacacagccctccctgcctgctgaccttggagctggggcagaggtcagagacctctctgggcccatgccacctccaacatccactcgaccccttggaatttttcggtggagaggagcagaggttgtcctggcgtggtttaggtagtgtgagaggggaatgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagtgaatccggactctaaggtaaatataaaatttttaagtgtataatgtgttaaactactgattctaattgtttctctcttttagattccaacctttggaactga

SEQ ID NO: 76 - DNA, 556 bp, Artificial Sequence
  Description of Artificial Sequence Synthetic polynucleotide
ccctaaaatgggcaaacattgcaagcagcaaacagcaaacacacagccctccctgcctgctgaccttggagctggggcagaggtcagagacctctctgggcccatgccacctccaacatccactcgaccccttggaatttcggtggagaggagcagaggttgtcctggcgtggtttaggtagtgtgagaggggaatgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttaaatacggacgaggacactcgagggccctgtctcctcagcttcaggcaccaccactgacctgggacagtgaatccggacatcgattctaaggtaaatataaaatttttaagtgtataatttgttaaactactgattctaattgtttctctcttttagattccaacctttggaactga

SEQ ID NO: 77 - DNA, 1179 bp, Artificial Sequence
  Description of Artificial Sequence Synthetic polynucleotide
ggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggtctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtga

SEQ ID NO: 78 - DNA, 141 bp, Artificial Sequence
  Description of Artificial Sequence Synthetic polynucleotide
aataaacgataacgccgttggtggcgtgaggcatgtaaaaggttacatcattatcttgttcgccatccggttggtataaatagacgttcatgttggtttttgtttcagttgcaagttggctgcggcgcgcgcagcaccttt

SEQ ID NO: 79 - DNA, 317 bp, Artificial Sequence
  Description of Artificial Sequence Synthetic polynucleotide
ggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaa

SEQ ID NO: 80 - DNA, 241 bp, Artificial Sequence
  Description of Artificial Sequence Synthetic polynucleotide
gagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggac

SEQ ID NO: 81 - DNA, 215 bp, Artificial Sequence
  Description of Artificial Sequence Synthetic polynucleotide
gaacgctgacgtcatcaacccgctccaaggaatcgcgggcccagtgtcactaggcgggaacacccagcgcgcgtgcgccctggcaggaagatggctgtgagggacaggggagtggcgccctgcaatatttgcatgtcgctatgtgttctgggaaatcaccataaacgtgaaatgtctttggatttgggaatcgtataagaactgtatgagaccac

SEQ ID NO: 82 - DNA, 546 bp, Artificial Sequence
  Description of Artificial Sequence Synthetic polynucleotide
ccctaaaatgggcaaacattgcaagcagcaaacagcaaacacacagccctccctgcctgctgaccttggagctggggcagaggtcagagacctctctgggcccatgccacctccaacatccactcgaccccttggaatttttcggtggagaggagcagaggttgtcctggcgtggtttaggtagtgtgagaggggaatgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagtgaatccggactctaaggtaaatataaaatttttaagtgtataatgtgttaaactactgattctaattgtttctctcttttagattccaacctttggaactg

SEQ ID NO: 83 - DNA, 576 bp, Artificial Sequence
  Description of Artificial Sequence Synthetic polynucleotide
tagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcag

SEQ ID NO: 84 - DNA, 150 bp, Artificial Sequence
  Description of Artificial Sequence Synthetic polynucleotide
ataaacgataacgccgttggtggcgtgaggcatgtaaaaggttacatcattatcttgttcgccatccggttggtataaatagacgttcatgttggtttttgtttcagttgcaagttggctgcggcgcgcgcagcacctttgcggccatct

SEQ ID NO: 85 - DNA, 1313 bp, Artificial Sequence
  Description of Artificial Sequence Synthetic polynucleotide
ggagccgagagtaattcatacaaaaggagggatcgccttcgcaaggggagagcccagggaccgtccctaaattctcacagacccaaatccctgtagccgccccacgacagcgcgaggagcatgcgcccagggctgagcgcgggtagatcagagcacacaagctcacagtccccggcggtggggggaggggcgcgctgagcgggggccagggagctggcgcggggcaaactgggaaagtggtgtcgtgtgctggctccgccctcttcccgagggtgggggagaacggtatataagtgcggtagtcgccttggacgttctttttcgcaacgggtttgccgtcagaacgcaggtgagtggcgggtgtggcttccgcgggccccggagctggagccctgctctgagcgggccgggctgatatgcgagtgtcgtccgcagggtttagctgtgagcattcccacttcgagtggcgggcggtgcgggggtgagagtgcgaggcctagcggcaaccccgtagcctcgcctcgtgtccggcttgaggcctagcgtggtgtccgccgccgcgtgccactccggccgcactatgcgttttttgtccttgctgccctcgattgccttccagcagcatgggctaacaaagggagggtgtggggctcactcttaaggagcccatgaagcttacgttggataggaatggaagggcaggaggggcgactggggcccgcccgccttcggagcacatgtccgacgccacctggatggggcgaggcctgtggctttccgaagcaatcgggcgtgagtttagcctacctgggccatgtggccctagcactgggcacggtctggcctggcggtgccgcgttcccttgcctcccaacaagggtgaggccgtcccgcccggcaccagttgcttgcgcggaaagatggccgctcccggggccctgttgcaaggagctcaaaatggaggacgcggcagcccggtggagcgggcgggtgagtcacccacacaaaggaagagggccttgcccctcgccggccgctgcttcctgtgaccccgtggtctatcggccgcatagtcacctcgggcttctcttgagcaccgctcgtcgcggcggggggaggggatctaatggcgttggagtttgttcacatttggtgggtggagactagtcaggccagcctggcgctggaagtcattcttggaatttgcccctttgagtttggagcgaggctaattctcaagcctcttagcggttcaaaggtattttctaaacccgtttccaggtgttgtgaaagccaccgctaattcaaagcaa

SEQ ID NO: 86 - DNA, 213 bp, Artificial Sequence
  Description of Artificial Sequence Synthetic polynucleotide
taagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggta

SEQ ID NO: 87 - protein, 7 aa, Artificial Sequence
  Description of Artificial Sequence Synthetic peptide
PKKKRKV

SEQ ID NO: 88 - protein, 19 aa, Artificial Sequence
  Description of Artificial Sequence Synthetic peptide
MDWTWRILFLVAAATGAHS

SEQ ID NO: 89 - protein, 19 aa, Artificial Sequence
  Description of Artificial Sequence Synthetic peptide
MLPSQLIGFLLLWVPASRG

SEQ ID NO: 90 - protein, 7 aa, Simian virus 40
PKKKRKV

SEQ ID NO: 91 - DNA, 21 bp, Simian virus 40
cccaagaagaagaggaaggtg

SEQ ID NO: 92 - protein, 16 aa, Unknown
  Description of Unknown Nucleoplasmin bipartite NLS sequence
KRPAATKKAGQAKKKK

SEQ ID NO: 93 - protein, 9 aa, Unknown
  Description of Unknown C-myc NLS sequence
PAAKRVKLD

SEQ ID NO: 94 - protein, 11 aa, Unknown
  Description of Unknown C-myc NLS sequence
RQRRNELKRSP

SEQ ID NO: 95 - protein, 38 aa, Homo sapiens
NQSSNFGPMKGGNFGGRSSGPYGGGGQYFAKPRNQGGY

SEQ ID NO: 96 - protein, 42 aa, Unknown
  Description of Unknown IBB domain from importin-alpha sequence
RMRIZFKNKGKDTAELRRRRVEVSVELRKAKKDEQILKRRNV

SEQ ID NO: 97 - protein, 8 aa, Unknown
  Description of Unknown Myoma T protein sequence
VSRKRPRP

SEQ ID NO: 98 - protein, 8 aa, Unknown
  Description of Unknown Myoma T protein sequence
PPKKARED

SEQ ID NO: 99 - protein, 8 aa, Homo sapiens
PQPKKKPL

SEQ ID NO: 100 - protein, 12 aa, Mus musculus
SALIKKKKKMAP

SEQ ID NO: 101 - DNA, 70 bp, Artificial Sequence
  Description of Artificial Sequence Synthetic oligonucleotide
gcgcgctcgctcgctcactgaggccgcccgggaaacccgggcgtgcgcctcagtgagcgagcgagcgcgc

SEQ ID NO: 102 - DNA, 70 bp, Artificial Sequence
  Description of Artificial Sequence Synthetic oligonucleotide
gcgcgctcgctcgctcactgaggcgcacgcccgggtttcccgggcggcctcagtgagcgagcgagcgcgc

SEQ ID NO: 103 - DNA, 72 bp, Artificial Sequence
  Description of Artificial Sequence Synthetic oligonucleotide
gcgcgctcgctcgctcactgaggccgtcgggcgacctttggtcgcccggcctcagtgagcgagcgagcgcgc

SEQ ID NO: 104 - DNA, 72 bp, Artificial Sequence
  Description of Artificial Sequence Synthetic oligonucleotide
gcgcgctcgctcgctcactgaggccgggcgaccaaaggtcgcccgacggcctcagtgagcgagcgagcgcgc

SEQ ID NO: 105 - DNA, 72 bp, Artificial Sequence
  Description of Artificial Sequence Synthetic oligonucleotide
gcgcgctcgctcgctcactgaggccgcccgggcaaagcccgggcgtcggcctcagtgagcgagcgagcgcgc

SEQ ID NO: 106 - DNA, 72 bp, Artificial Sequence
  Description of Artificial Sequence Synthetic oligonucleotide
gcgcgctcgctcgctcactgaggccgacgcccgggctttgcccgggcggcctcagtgagcgagcgagcgcgc

SEQ ID NO: 107 - DNA, 83 bp, Artificial Sequence
  Description of Artificial Sequence Synthetic oligonucleotide
gcgcgctcgctcgctcactgaggccgcccgggcaaagcccgggcgtcgggctttgcccggcctcagtgagcgagcgagcgcgc

SEQ ID NO: 108 - DNA, 83 bp, Artificial Sequence
  Description of Artificial Sequence Synthetic oligonucleotide
gcgcgctcgctcgctcactgaggccgggcaaagcccgacgcccgggctttgcccgggcggcctcagtgagcgagcgagcgcgc

SEQ ID NO: 109 - DNA, 77 bp, Artificial Sequence
  Description of Artificial Sequence Synthetic oligonucleotide
gcgcgctcgctcgctcactgaggccgaaacgtcgggcgacctttggtcgcccggcctcagtgagcgagcgagcgcgc

SEQ ID NO: 110 - DNA, 77 bp, Artificial Sequence
  Description of Artificial Sequence Synthetic oligonucleotide
gcgcgctcgctcgctcactgaggccgggcgaccaaaggtcgcccgacgtttcggcctcagtgagcgagcgagcgcgc

SEQ ID NO: 111 - DNA, 51 bp, Artificial Sequence
  Description of Artificial Sequence Synthetic oligonucleotide
gcgcgctcgctcgctcactgaggcaaagcctcagtgagcgagcgagcgcgc

SEQ ID NO: 112 - DNA, 51 bp, Artificial Sequence
  Description of Artificial Sequence Synthetic oligonucleotide
gcgcgctcgctcgctcactgaggctttgcctcagtgagcgagcgagcgcgc

SEQ ID NO: 113 - DNA, 80 bp, Artificial Sequence
  Description of Artificial Sequence Synthetic oligonucleotide
gcgcgctcgctcgctcactgaggccgcccgggcgtcgggcgacctttggtcgcccggcctcagtgagcgagcgagcgcgc

SEQ ID NO: 114 - DNA, 80 bp, Artificial Sequence
  Description of Artificial Sequence Synthetic oligonucleotide
gcgcgctcgctcgctcactgaggccgggcgaccaaaggtcgcccgacgcccgggcggcctcagtgagcgagcgagcgcgc

SEQ ID NO: 115 - DNA, 79 bp, Artificial Sequence
  Description of Artificial Sequence Synthetic oligonucleotide
gcgcgctcgctcgctcactgaggcgcccgggcgtcgggcgacctttggtcgcccggcctcagtgagcgagcgagcgcgc

SEQ ID NO: 116 - DNA, 79 bp, Artificial Sequence
  Description of Artificial Sequence Synthetic oligonucleotide
gcgcgctcgctcgctcactgaggccgggcgaccaaaggtcgcccgacgcccgggcgcctcagtgagcgagcgagcgcgc

SEQ ID NO: 117 - protein, 5 aa, Influenza virus
DRLRR

SEQ ID NO: 118 - protein, 7 aa, Influenza virus
PKQKKRK

SEQ ID NO: 119 - protein, 10 aa, Hepatitis delta virus
RKLKKKIKKL

SEQ ID NO: 120 - protein, 10 aa, Mus musculus
REKKKFLKRR

SEQ ID NO: 121 - protein, 20 aa, Homo sapiens
KRKGDEVDGVDEVAKKKSKK

SEQ ID NO: 122 - protein, 17 aa, Homo sapiens
RKCLQAGMNLEARKTKK

SEQ ID NO: 123 - DNA, 8 bp, Artificial Sequence
  Description of Artificial Sequence Synthetic oligonucleotide
gtttaaac

SEQ ID NO: 124 - DNA, 8 bp, Artificial Sequence
  Description of Artificial Sequence Synthetic oligonucleotide
ttaattaa

SEQ ID NO: 125 - DNA, 141 bp, Artificial Sequence; identical to SEQ ID NO: 78
  Description of Artificial Sequence Synthetic polynucleotide

SEQ ID NO: 126 - DNA, 317 bp, Artificial Sequence; identical to SEQ ID NO: 79
  Description of Artificial Sequence Synthetic polynucleotide

SEQ ID NO: 127 - DNA, 72 bp, Artificial Sequence; identical to SEQ ID NO: 104
  Description of Artificial Sequence Synthetic oligonucleotide

SEQ ID NO: 128 - DNA, 60 bp, Artificial Sequence
  Description of Artificial Sequence Synthetic oligonucleotide
gagacagacacactcctgctatgggtactgctgctctgggttccaggttccactggtgac

SEQ ID NO: 129 - DNA, 1260 bp, Adeno-associated virus - 2
atggagctggtcgggtggctcgtggacaaggggattacctcggagaagcagtggatccaggaggaccaggcctcatacatctccttcaatgcggcctccaactcgcggtcccaaatcaaggctgccttggacaatgcgggaaagattatgagcctgactaaaaccgcccccgactacctggtgggccagcagcccgtggaggacatttccagcaatcggatttataaaattttggaactaaacgggtacgatccccaatatgcggcttccgtctttctgggatgggccacgaaaaagttcggcaagaggaacaccatctggctgtttgggcctgcaactaccgggaagaccaacatcgcggaggccatagcccacactgtgcccttctacgggtgcgtaaactggaccaatgagaactttcccttcaacgactgtgtcgacaagatggtgatctggtgggaggaggggaagatgaccgccaaggtcgtggagtcggccaaagccattctcggaggaagcaaggtgcgcgtggaccagaaatgcaagtcctcggcccagatagacccgactcccgtgatcgtcacctccaacaccaacatgtgcgccgtgattgacgggaactcaacgaccttcgaacaccagcagccgttgcaagaccggatgttcaaatttgaactcacccgccgtctggatcatgactttgggaaggtcaccaagcaggaagtcaaagactttttccggtgggcaaaggatcacgtggttgaggtggagcatgaattctacgtcaaaaagggtggagccaagaaaagacccgcccccagtgacgcagatataagtgagcccaaacgggtgcgcgagtcagttgcgcagccatcgacgtcagacgcggaagcttcgatcaactacgcagacaggtaccaaaacaaatgttctcgtcacgtgggcatgaatctgatgctgtttccctgcagacaatgcgagagaatgaatcagaattcaaatatctgcttcactcacggacagaaagactgtttagagtgctttcccgtgtcagaatctcaacccgtttctgtcgtcaaaaaggcgtatcagaaactgtgctacattcatcatatcatgggaaaggtgccagacgcttgcactgcctgcgatctggtcaatgtggatttggatgactgcatctttgaacaataaatgatttaaatcaggtatggctgccgatggttatcttccagattggctcgaggacactctctctga

SEQ ID NO: 130 - DNA, 1932 bp, Adeno-associated virus - 2
atgccggggttttacgagattgtgattaaggtccccagcgaccttgacgagcatctgcccggcatttctgacagctttgtgaactgggtggccgagaaggaatgggagttgccgccagattctgacatggatctgaatctgattgagcaggcacccctgaccgtggccgagaagctgcagcgcgactttctgacggaatggcgccgtgtgagtaaggccccggaggcccttttctttgtgcaatttgagaagggagagagctacttccacatgcacgtgctcgtggaaaccaccggggtgaaatccatggttttgggacgtttcctgagtcagattcgcgaaaaactgattcagagaatttaccgcgggatcgagccgactttgccaaactggttcgcggtcacaaagaccagaaatggcgccggaggcgggaacaaggtggtggatgagtgctacatccccaattacttgctccccaaaacccagcctgagctccagtgggcgtggactaatatggaacagtatttaagcgcctgtttgaatctcacggagcgtaaacggttggtggcgcagcatctgacgcacgtgtcgcagacgcaggagcagaacaaagagaatcagaatcccaattctgatgcgccggtgatcagatcaaaaacttcagccaggtacatggagctggtcgggtggctcgtggacaaggggattacctcggagaagcagtggatccaggaggaccaggcctcatacatctccttcaatgcggcctccaactcgcggtcccaaatcaaggctgccttggacaatgcgggaaagattatgagcctgactaaaaccgcccccgactacctggtgggccagcagcccgtggaggacatttccagcaatcggatttataaaattttggaactaaacgggtacgatccccaatatgcggcttccgtctttctgggatgggccacgaaaaagttcggcaagaggaacaccatctggctgtttgggcctgcaactaccgggaagaccaacatcgcggaggccatagcccacactgtgcccttctacgggtgcgtaaactggaccaatgagaactttcccttcaacgactgtgtcgacaagatggtgatctggtgggaggaggggaagatgaccgccaaggtcgtggagtcggccaaagccattctcggaggaagcaaggtgcgcgtggaccagaaatgcaagtcctcggcccagatagacccgactcccgtgatcgtcacctccaacaccaacatgtgcgccgtgattgacgggaactcaacgaccttcgaacaccagcagccgttgcaagaccggatgttcaaatttgaactcacccgccgtctggatcatgactttgggaaggtcaccaagcaggaagtcaaagactttttccggtgggcaaaggatcacgtggttgaggtggagcatgaattctacgtcaaaaagggtggagccaagaaaagacccgcccccagtgacgcagatataagtgagcccaaacgggtgcgcgagtcagttgcgcagccatcgacgtcagacgcggaagcttcgatcaactacgcagacaggtaccaaaacaaatgttctcgtcacgtgggcatgaatctgatgctgtttccctgcagacaatgcgagagaatgaatcagaattcaaatatctgcttcactcacggacagaaagactgtttagagtgctttcccgtgtcagaatctcaacccgtttctgtcgtcaaaaaggcgtatcagaaactgtgctacattcatcatatcatgggaaaggtgccagacgcttgcactgcctgcgatctggtcaatgtggatttggatgactgcatctttgaacaataaatgatttaaatcaggtatggctgccgatggttatcttccagattggctcgaggacactctctctga

SEQ ID NO: 131 - DNA, 1876 bp, Artificial Sequence
  Description of Artificial Sequence Synthetic polynucleotide
cgcagccaccatggcggggttttacgagattgtgattaaggtccccagcgaccttgacgggcatctgcccggcatttctgacagctttgtgaactgggtggccgagaaggaatgggagttgccgccagattctgacatggatctgaatctgattgagcaggcacccctgaccgtggccgagaagctgcagcgcgactttctgacggaatggcgccgtgtgagtaaggccccggaggcccttttctttgtgcaatttgagaagggagagagctacttccacatgcacgtgctcgtggaaaccaccggggtgaaatccatggttttgggacgtttcctgagtcagattcgcgaaaaactgattcagagaatttaccgcgggatcgagccgactttgccaaactggttcgcggtcacaaagaccagaaatggcgccggaggcgggaacaaggtggtggatgagtgctacatccccaattacttgctccccaaaacccagcctgagctccagtgggcgtggactaatatggaacagtatttaagcgcctgtttgaatctcacggagcgtaaacggttggtggcgcagcatctgacgcacgtgtcgcagacgcaggagcagaacaaagagaatcagaatcccaattctgatgcgccggtgatcagatcaaaaacttcagccaggtacatggagctggtcgggtggctcgtggacaaggggattacctcggagaagcagtggatccaggaggaccaggcctcatacatctccttcaatgcggcctccaactcgcggtcccaaatcaaggctgccttggacaatgcgggaaagattatgagcctgactaaaaccgcccccgactacctggtgggccagcagcccgtggaggacatttccagcaatcggatttataaaattttggaactaaacgggtacgatccccaatatgcggcttccgtctttctgggatgggccacgaaaaagttcggcaagaggaacaccatctggctgtttgggcctgcaactaccgggaagaccaacatcgcggaggccatagcccacactgtgcccttctacgggtgcgtaaactggaccaatgagaactttcccttcaacgactgtgtcgacaagatggtgatctggtgggaggaggggaagatgaccgccaaggtcgtggagtcggccaaagccattctcggaggaagcaaggtgcgcgtggaccagaaatgcaagtcctcggcccagatagacccgactcccgtgatcgtcacctccaacaccaacatgtgcgccgtgattgacgggaactcaacgaccttcgaacaccagcagccgttgcaagaccggatgttcaaatttgaactcacccgccgtctggatcatgactttgggaaggtcaccaagcaggaagtcaaagactttttccggtgggcaaaggatcacgtggttgaggtggagcatgaattctacgtcaaaaagggtggagccaagaaaagacccgcccccagtgacgcagatataagtgagcccaaacgggtgcgcgagtcagttgcgcagccatcgacgtcagacgcggaagcttcgatcaactacgcagacaggtaccaaaacaaatgttctcgtcacgtgggcatgaatctgatgctgtttccctgcagacaatgcgagagaatgaatcagaattcaaatatctgcttcactcacggacagaaagactgtttagagtgctttcccgtgtcagaatctcaacccgtttctgtcgtcaaaaaggcgtatcagaaactgtgctacattcatcatatcatgggaaaggtgccagacgcttgcactgcctgcgatctggtcaatgtggatttggatgactgcatctttgaacaataa

SEQ ID NO: 132 - DNA, 1194 bp, Artificial Sequence
  Description of Artificial Sequence Synthetic polynucleotide
atggagctggtcgggtggctcgtggacaaggggattacctcggagaagcagtggatccaggaggaccaggcctcatacatctccttcaatgcggcctccaactcgcggtcccaaatcaaggctgccttggacaatgcgggaaagattatgagcctgactaaaaccgcccccgactacctggtgggccagcagcccgtggaggacatttccagcaatcggatttataaaattttggaactaaacgggtacgatccccaatatgcggcttccgtctttctgggatgggccacgaaaaagttcggcaagaggaacaccatctggctgtttgggcctgcaactaccgggaagaccaacatcgcggaggccatagcccacactgtgcccttctacgggtgcgtaaactggaccaatgagaactttcccttcaacgactgtgtcgacaagatggtgatctggtgggaggaggggaagatgaccgccaaggtcgtggagtcggccaaagccattctcggaggaagcaaggtgcgcgtggaccagaaatgcaagtcctcggcccagatagacccgactcccgtgatcgtcacctccaacaccaacatgtgcgccgtgattgacgggaactcaacgaccttcgaacaccagcagccgttgcaagaccggatgttcaaatttgaactcacccgccgtctggatcatgactttgggaaggtcaccaagcaggaagtcaaagactttttccggtgggcaaaggatcacgtggttgaggtggagcatgaattctacgtcaaaaagggtggagccaagaaaagacccgcccccagtgacgcagatataagtgagcccaaacgggtgcgcgagtcagttgcgcagccatcgacgtcagacgcggaagcttcgatcaactacgcagaccgctaccaaaacaaatgttctcgtcacgtgggcatgaatctgatgctgtttccctgcagacaatgcgagagaatgaatcagaattcaaatatctgcttcactcacggacagaaagactgtttagagtgctttcccgtgtcagaatctcaacccgtttctgtcgtcaaaaaggcgtatcagaaactgtgctacattcatcatatcatgggaaaggtgccagacgcttgcactgcctgcgatctggtcaatgtggatttggatgactgcatctttgaacaataa

SEQ ID NO: 133 - DNA, 1876 bp, Artificial Sequence; identical to SEQ ID NO: 131
  Description of Artificial Sequence Synthetic polynucleotide

SEQ ID NO: 134 - DNA, 51 bp, Artificial Sequence
  Description of Artificial Sequence Synthetic oligonucleotide
ctaggccgcccgggcaaagcccgggcgtcgggcgacctttggtcgcccggc

SEQ ID NO: 135 - DNA, 65 bp, Artificial Sequence
  Description of Artificial Sequence Synthetic oligonucleotide
ctaggactgaggccgcccgggcaaagcccgggcgtcgggcgacctttggtcgcccggcctcagtc

SEQ ID NO: 136 - DNA, 67 bp, Artificial Sequence
  Description of Artificial Sequence Synthetic oligonucleotide
ggactgaggccgcccgggcaaagcccgggcgtcgggcgacctttggtcgcccggcctcagtcctgca

SEQ ID NO: 137 - DNA, 41 bp, Artificial Sequence
  Description of Artificial Sequence Synthetic oligonucleotide
gtgcgggcgaccaaaggtcgcccgacgcccgggcgcactca

SEQ ID NO: 138 - DNA, 56 bp, Artificial Sequence
  Description of Artificial Sequence Synthetic oligonucleotide
ggactgaggccgggcgaccaaaggtcgcccgacgcccgggcggcctcagtcctgca

SEQ ID NO: 139 - DNA, 54 bp, Artificial Sequence
  Description of Artificial Sequence Synthetic oligonucleotide
ctaggactgaggccgcccgggcgtcgggcgacctttggtcgcccggcctcagtc

SEQ ID NO: 140 - DNA, 48 bp, Artificial Sequence
  Description of Artificial Sequence Synthetic oligonucleotide
ggactgaggccgggcgaccaaaggtcgcccgacggcctcagtcctgca

SEQ ID NO: 141 - DNA, 46 bp, Artificial Sequence
  Description of Artificial Sequence Synthetic oligonucleotide
ctaggactgaggccgtcgggcgacctttggtcgcccggcctcagtc

SEQ ID NO: 142 - DNA, 67 bp, Artificial Sequence
  Description of Artificial Sequence Synthetic oligonucleotide
ggactgaggcccgggcgaccaaaggtcgcccgacgcccgggctttgcccgggcgcctcagtcctgca

SEQ ID NO: 143 - DNA, 47 bp, Artificial Sequence
  Description of Artificial Sequence Synthetic oligonucleotide
atacctaggcacgcgtgttactagttattaatagtaatcaattacgg

SEQ ID NO: 144 - DNA, 29 bp, Artificial Sequence
  Description of Artificial Sequence Synthetic oligonucleotide
atacctaggggccgcacgcgtgttactag

SEQ ID NO: 145 - DNA, 42 bp, Artificial Sequence
  Description of Artificial Sequence Synthetic oligonucleotide
atacactcagtgcctgcaggcacgtggtccggagatccagac

SEQ ID NO: 146 - DNA, 3754 bp, Artificial Sequence
  Description of Artificial Sequence Synthetic polynucleotide
cctaggtgagcgagcgagcgcgcagagagggagtggccaactccatcactaggggttccttgtagttaatgattaacccgccatgctacttatcgcggccgctcaatattggccattagccatattattcattggttatatagcataaatcaatattggctattggccattgcatacgttgtatctatatcataatatgtacatttatattggctcatgtccaatatgaccgccatgttggcattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtccgccccctattgacgtcaatgacggtaaatggcccgcctggcattatgcccagtacatgaccttacgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgacgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcgggggggagcggctcggggggtgcgtgcgtgtgtgtgtgcgtggggagcgccgcgtgcggcccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcggcggtcgggctgtaacccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttttagccttcttctttttcctacagctcctgggcaacgtgctggttattgtgctgtctcatcatttgtcgacagaattcctcgaagatccgaaggggttcaagcttggcattccggtactgttggtaaagccagtttaaacgccgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaattaattaagagcatcttaccgccatttattcccatatttgttctgtttttcttgatttgggtatacatttaaatgttaataaaacaaaatggtggggcaatcatttacatttttagggatatgtaattactagttcaggtgtattgccacaagacaaacatgttaagaaactttcccgttatttacgctctgttcctgttaatcaacctctggattacaaaatttgtgaaagattgactgatattcttaactatgttgctccttttacgctgtgtggatatgctgctttatagcctctgtatctagctattgcttcccgtacggctttcgttttctcctccttgtataaatcctggttgctgtctcttttagaggagttgtggcccgttgtccgtcaacgtggcgtggtgtgctctgtgtttgctgacgcaacccccactggctggggcattgccaccacctgtcaactcctttctgggactttcgctttccccctcccgatcgccacggcagaactcatcgccgcctgccttgcccgctgctggacaggggctaggttgctgggcactgataattccgtggtgttgtctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggctctagagcatggctacgtagataagtagcatggcgggttaatcattaactacacctgcagcaggaacccctagtgatggagttggccactccctctctgcgcgctcgctcgctccctgcaggactgaggccgggcgaccaaaggtcgcccgacgcccgggcggcctcagtcctgcagg

SEQ ID NO: 147 - DNA, 8418 bp, Artificial Sequence
  Description of Artificial Sequence Synthetic polynucleotide
ggcagctgcgcgctcgctcgctcacctaggccgcccgggcaaagcccgggcgtcgggcgacctttggtcgcccggcctaggtgagcgagcgagcgcgcagagagggagtggccaactccatcactaggggttccttgtagttaatgattaacccgccatgctacttatcgcggccgctcaatattggccattagccatattattcattggttatatagcataaatcaatattggctattggccattgcatacgttgtatctatatcataatatgtacatttatattggctcatgtccaatatgaccgccatgttggcattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtccgccccctattgacgtcaatgacggtaaatggcccgcctggcattatgcccagtacatgaccttacgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgacgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcgggggggagcggctcggggggtgcgtgcgtgtgtgtgtgcgtggggagcgccgcgtgcggcccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcggcggtcgggctgtaacccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttttagccttcttctttttcctacagctcctgggcaacgtgctggttattgtgctgtctcatcatttgtcgacagaattcctcgaagatccgaaggggttcaagcttggcattccggtactgttggtaaagccagtttaaacgccgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaattaattaagagcatcttaccgccatttattcccatatttgttctgtttttcttgatttgggtatacatttaaatgttaataaaacaaaatggtggggcaatcatttacatttttagggatatgtaattactagttcaggtgtattgccacaagacaaacatgttaagaaactttcccgttatttacgctctgttcctgttaatcaacctctggattacaaaatttgtgaaagattgactgatattcttaactatgttgctccttttacgctgtgtggatatgctgctttatagcctctgtatctagctattgcttcccgtacggctttcgttttctcctccttgtataaatcctggttgctgtctcttttagaggagttgtggcccgttgtccgtcaacgtggcgtggtgtgctctgtgtttgctgacgcaacccccactggctggggcattgccaccacctgtcaactcctttctgggactttcgctttccccctcccgatcgccacggcagaactcatcgccgcctgccttgcccgctgctggacaggggctaggttgctgggcactgataattccgtggtgttgtctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggctctagagcatggctacgtagataagtagcatggcgggttaatcattaactacacctgcagcaggaacccctagtgatggagttggccactccctctctgcgcgctcgctcgctccctgcaggactgaggccgggcgaccaaaggtcgcccgacgcccgggcggcctcagtcctgcagggagcgagcgagcgcgcagctgcctgcacgggcgcgccggtaccgggagatgggggaggctaactgaaacacggaaggagacaataccggaaggaacccgcgctatgacggcaataaaaagacagaataaaacgcacgggtgttgggtcgtttgttcataaacgcggggttcggtcccagggctggcactctgtcgataccccaccgagaccccattgggaccaatacgcccgcgtttcttccttttccccaccccaacccccaagttcgggtgaaggcccagggctcgcagccaacgtcggggcggcaagccctgccatagccactacgggtacgtaggccaaccactagaactatagctagagtcctgggcgaacaaacgatgctcgccttccagaaaaccgaggatgcgaaccacttcatccggggtcagcaccaccggcaagcgccgcgacggccgaggtctaccgatctcctgaagccagggcagatccgtgcacagcaccttgccgtagaagaacagcaaggccgccaatgcctgacgatgcgtggagaccgaaaccttgcgctcgttcgccagccaggacagaaatgcctcgacttcgctgctgcccaaggttgccgggtgacgcacaccgtggaaacggatgaaggcacgaacccagttgacataagcctgttcggttcgtaaactgtaatgcaagtagcgtatgcgctcacgcaactggtccagaaccttgaccgaacgcagcggtggtaacggcgcagtggcggttttcatggcttgttatgactgtttttttgtacagtctatgcctcgggcatccaagcagcaagcgcgttacgccgtgggtcgatgtttgatgttatggagcagcaacgatgttacgcagcagcaacgatgttacgcagcagggcagtcgccctaaaacaaagttaggtggctcaagtatgggcatcattcgcacatgtaggctcggccctgaccaagtcaaatccatgcgggctgctcttgatcttttcggtcgtgagttcggagacgtagccacctactcccaacatcagccggactccgattacctcgggaacttgctccgtagtaagacattcatcgcgcttgctgccttcgaccaagaagcggttgttggcgctctcgcggcttacgttctgcccaggtttgagcagccgcgtagtgagatctatatctatgatctcgcagtctccggcgagcaccggaggcagggcattgccaccgcgctcatcaatctcctcaagcatgaggccaacgcgcttggtgcttatgtgatctacgtgcaagcagattacggtgacgatcccgcagtggctctctatacaaagttgggcatacgggaagaagtgatgcactttgatatcgacccaagtaccgccacctaacaattcgttcaagccgagatcggcttcccggccgcggagttgttcggtaaattgtcacaacgccgcgaatatagtctttaccatgcccttggccacgcccctctttaatacgacgggcaatttgcacttcagaaaatgaagagtttgctttagccataacaaaagtccagtatgctttttcacagcataactggactgatttcagtttacaactattctgtctagtttaagactttattgtcatagtttagatctattttgttcagtttaagactttattgtccgcccacacccgcttacgcagggcatccatttattactcaaccgtaaccgattttgccaggttacgcggctggtctgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgaaattgtaaacgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcccattcgccattcaggctgcaaataagcgttgatattcagtcaattacaaacattaataacgaagagatgacagaaaaattttcattctgtgacagagaaaaagtagccgaagatgacggtttgtcacatggagttggcaggatgtttgattaaaaacataacaggaagaaaaatgccccgctgtgggcggacaaaatagttgggaactgggaggggtggaaatggagtttttaaggattatttagggaagagtgacaaaatagatgggaactgggtgtagcgtcgtaagctaatacgaaaattaaaaatgacaaaatagtttggaactagatttcacttatctggttcggatctcctagtgagctccctgca

SEQ ID NO: 148 - DNA, 225 bp, Artificial Sequence; identical to SEQ ID NO: 68
  Description of Artificial Sequence Synthetic polynucleotide

SEQ ID NO: 149 - DNA, 1177 bp, Artificial Sequence; identical to SEQ ID NO: 74
  Description of Artificial Sequence Synthetic polynucleotide

SEQ ID NO: 150 - DNA, 1326 bp, Artificial Sequence
  Description of Artificial Sequence Synthetic polynucleotide
ctgcagggcccactagtggagccgagagtaattcatacaaaaggagggatcgccttcgcaaggggagagcccagggaccgtccctaaattctcacagacccaaatccctgtagccgccccacgacagcgcgaggagcatgcgcccagggctgagcgcgggtagatcagagcacacaagctcacagtccccggcggtggggggaggggcgcgctgagcgggggccagggagctggcgcggggcaaactgggaaagtggtgtcgtgtgctggctccgccctcttcccgagggtgggggagaacggtatataagtgcggtagtcgccttggacgttctttttcgcaacgggtttgccgtcagaacgcaggtgagtggcgggtgtggcttccgcgggccccggagctggagccctgctctgagcgggccgggctgatatgcgagtgtcgtccgcagggtttagctgtgagcattcccacttcgagtggcgggcggtgcgggggtgagagtgcgaggcctagcggcaaccccgtagcctcgcctcgtgtccggcttgaggcctagcgtggtgtccgccgccgcgtgccactccggccgcactatgcgttttttgtccttgctgccctcgattgccttccagcagcatgggctaacaaagggagggtgtggggctcactcttaaggagcccatgaagcttacgttggataggaatggaagggcaggaggggcgactggggcccgcccgccttcggagcacatgtccgacgccacctggatggggcgaggcctgtggctttccgaagcaatcgggcgtgagtttagcctacctgggccatgtggccctagcactgggcacggtctggcctggcggtgccgcgttcccttgcctcccaacaagggtgaggccgtcccgcccggcaccagttgcttgcgcggaaagatggccgctcccggggccctgttgcaaggagctcaaaatggaggacgcggcagcccggtggagcgggcgggtgagtcacccacacaaaggaagagggccttgcccctcgccggccgctgcttcctgtgaccccgtggtctatcggccgcatagtcacctcgggcttctcttgagcaccgctcgtcgcggcggggggaggggatctaatggcgttggagtttgttcacatttggtgggtggagactagtcaggccagcctggcgctggaagtcattcttggaatttgcccctttgagtttggagcgaggctaattctcaagcctcttagcggttcaaaggtattttctaaacccgtttccaggtgttgtgaaagccaccgctaattcaaa

SEQ ID NO: 151 - DNA, 573 bp, Mus musculus
gtaagagttttatgttttttcatctctgcttgtatttttctagtaatggaagcctggtattttaaaatagttaaattttcctttagtgctgatttctagattattattactgttgttgttgttattattgtcattatttgcatctgagaacccttaggtggttatattattgatatatttttggtatctttgatgacaataatgggggattttgaaagcttagctttaaatttcttttaattaaaaaaaaatgctaggcagaatgactcaaattacgttggatacagttgaatttattacggtctcatagggcctgcctgctcgaccatgctatactaaaaattaaaagtgtgtgttactaattttataaatggagtttccatttatatttacctttatttcttatttaccattgtcttagtagatatttacaaacatgacagaaacactaaatcttgagtttgaatgcacagatataaacacttaacgggttttaaaaataataatgttggtgaaaaaatataactttgagtgtagcagagaggaaccattgccaccttcagattttcctgt

SEQ ID NO: 152 - DNA, 1993 bp, Mus musculus
acgatcgggaactggcatcttcagggagtagcttaggtcagtgaagagaagaacaaaaagcagcatattacagttagttgtcttcatcaatctttaaatatgttgtgtggtttttctctccctgtttccacagacaagagtgagatcgcccatcggtataatgatttgggagaacaacatttcaaaggcctgtaagttataatgctgaaagcccacttaatatttctggtagtattagttaaagttttaaaacacctttttccaccttgagtgtgagaattgtagagcagtgctgtccagtagaaatgtgtgcattgacagaaagactgtggatctgtgctgagcaatgtggcagccagagatcacaaggctatcaagcactttgcacatggcaagtgtaactgagaagcacacattcaaataatagttaattttaattgaatgtatctagccatgtgtggctagtagctcctttcctggagagagaatctggagcccacatctaacttgttaagtctggaatcttattttttatttctggaaaggtctatgaactatagttttgggggcagctcacttactaacttttaatgcaataagatctcatggtatcttgagaacattattttgtctctttgtagtactgaaaccttatacatgtgaagtaaggggtctatacttaagtcacatctccaaccttagtaatgttttaatgtagtaaaaaaatgagtaattaatttatttttagaaggtcaatagtatcatgtattccaaataacagaggtatatggttagaaaagaaacaattcaaaggacttatataatatctagccttgacaatgaataaatttagagagtagtttgcctgtttgcctcatgttcataaatctattgacacatatgtgcatctgcacttcagcatggtagaagtccatattcctttgcttggaaaggcaggtgttcccattacgcctcagagaatagctgacgggaagaggctttctagatagttgtatgaaagatatacaaaatctcgcaggtatacacaggcatgatttgctggttgggagagccacttgcctcatactgaggtttttgtgtctgcttttcagagtcctgattgccttttcccagtatctccagaaatgctcatacgatgagcatgccaaattagtgcaggaagtaacagactttgcaaagacgtgtgttgccgatgagtctgccgccaactgtgacaaatcccttgtgagtaccttctgattttgtggatctactttcctgctttctggaactctgtttcaaagccaatcatgactccatcacttaaggccccgggaacactgtggcagagggcagcagagagattgataaagccagggtgatgggaattttctgtgggactccatttcatagtaattgcagaagctacaatacactcaaaaagtctcaccacatgactgcccaaatgggagcttgacagtgacagtgacagtagatatgccaaagtggatgagggaaagaccacaagagctaaaccctgtaaaaagaactgtaggcaactaaggaatgcagagagaagaagttgccttggaagagcataccaactgcctctccaataccaatggtcatccctaaaacatacgtatgaataacatgcagactaagcaggctacatttaggaatatacatgtatttacataaatgtatatgcatgtaacaacaatgaatgaaaactgaggtcatggatctgaaagagagcaagggggcttacatgagagggtttggagggaggggttggagggagggaggtattattctttagttttacagggaacgtagtaaaaacataggcttctcccaaaggagcagagcccatgaggagctgtgcaaggttccccagcttgattttacctgctcctcaaattcccttgatttgtttttattataatgactttactcctagcttttagtgtcagatagaaaacatggaagg

SEQ ID NO: 153 - DNA, 1350 bp, Homo sapiens
taggaggctgaggcaggaggatcgcttgagcccaggagttcgagaccagcctgggcaacatagtgtgatcttgtatctataaaaataaacaaaattagcttggtgtggtggcgcctgtagtccccagccacttggaggggtgaggtgagaggattgcttgagcccgggatggtccaggctgcagtgagccatgatcgtgccactgcactccagcctgggcgacagagtgagaccctgtctcacaacaacaacaacaacaacaaaaaggctgagctgcaccatgcttgacccagtttcttaaaattgttgtcaaagcttcattcactccatggtgctatagagcacaagattttatttggtgagatggtgctttcatgaattcccccaacagagccaagctctccatctagtggacagggaagctagcagcaaaccttcccttcactacaaaacttcattgcttggccaaaaagagagttaattcaatgtagacatctatgtaggcaattaaaaacctattgatgtataaaacagtttgcattcatggagggcaactaaatacattctaggactttataaaagatcactttttatttatgcacagggtggaacaagatggattatcaagtgtcaagtccaatctatgacatcaattattatacatcggagccctgccaaaaaatcaatgtgaagcaaatcgcagcccgcctcctgcctccgctctactcactggtgttcatctttggttttgtgggcaacatgctggtcatcctcatcctgataaactgcaaaaggctgaagagcatgactgacatctacctgctcaacctggccatctctgacctgtttttccttcttactgtccccttctgggctcactatgctgccgcccagtgggactttggaaatacaatgtgtcaactcttgacagggctctattttataggcttcttctctggaatcttcttcatcatcctcctgacaatcgataggtacctggctgtcgtccatgctgtgtttgctttaaaagccaggacggtcacctttggggtggtgacaagtgtgatcacttgggtggtggctgtgtttgcgtctctcccaggaatcatctttaccagatctcaaaaagaaggtcttcattacacctgcagctctcattttccatacagtcagtatcaattctggaagaatttccagacattaaagatagtcatcttggggctggtcctgccgctgcttgtcatggtcatctgctactcgggaatcctaaaaactctgcttcggtgtcgaaatgagaagaagaggcacagggctgtgaggcttatcttcaccatcatgattgtttatttt

SEQ ID NO: 154 - DNA, 1223 bp, Homo sapiens
tgacagagactcttgggatgacgcactgctgcatcaaccccatcatctatgcctttgtcggggagaagttcagaaactacctcttagtcttcttccaaaagcacattgccaaacgcttctgcaaatgctgttctattttccagcaagaggctcccgagcgagcaagctcagtttacacccgatccactggggagcaggaaatatctgtgggcttgtgacacggactcaagtgggctggtgacccagtcagagttgtgcacatggcttagttttcatacacagcctgggctgggggtggggtgggagaggtcttttttaaaaggaagttactgttatagagggtctaagattcatccatttatttggcatctgtttaaagtagattagatcttttaagcccatcaattatagaaagccaaatcaaaatatgttgatgaaaaatagcaacctttttatctccccttcacatgcatcaagttattgacaaactctcccttcactccgaaagttccttatgtatatttaaaagaaagcctcagagaattgctgattcttgagtttagtgatctgaacagaaataccaaaattatttcagaaatgtacaactttttacctagtacaaggcaacatataggttgtaaatgtgtttaaaacaggtctttgtcttgctatggggagaaaagacatgaatatgattagtaaagaaatgacacttttcatgtgtgatttcccctccaaggtatggttaataagtttcactgacttagaaccaggcgagagacttgtggcctgggagagctggggaagcttcttaaatgagaaggaatttgagttggatcatctattgctggcaaagacagaagcctcactgcaagcactgcatgggcaagcttggctgtagaaggagacagagctggttgggaagacatggggaggaaggacaaggctagatcatgaagaaccttgacggcattgctccgtctaagtcatgagctgagcagggagatcctggttggtgttgcagaaggtttactctgtggccaaaggagggtcaggaaggatgagcatttagggcaaggagaccaccaacagccctcaggtcagggtgaggatggcctctgctaagctcaaggcgtgaggatgggaaggagggaggtattcgtaaggatgggaaggagggaggtattcgtgcagcatatgaggatgcagagtcagcagaactg

SEQ ID NO: 155 - DNA, 215 bp, Artificial Sequence
  Description of Artificial Sequence Synthetic polynucleotide
gaacgctgacgtcatcaacccgctccaaggaatcgcgggcccagtgtcactaggcgggaacacccagcgcgcgtgcgccctggcaggaagatggctgtgagggacaggggagtggcgccctgcaatatttgcatgtcgctatgtgttctgggaaatcaccataaacgtgaaatgtctttggatttgggaatcttataagttctgtatgagaccac

SEQ ID NO: 156 - DNA, 141 bp, Artificial Sequence
  Description of Artificial Sequence Synthetic polynucleotide
cctgcaggcagctgcgcgctcgctcgctcacctaggccgcccgggcaaagcccgggcgtcgggcgacctttggtcgcccggcctaggtgagcgagcgagcgcgcagagagggagtggccaactccatcactaggggttcct

SEQ ID NO: 157 - DNA, 19 bp, Artificial Sequence
  Description of Artificial Sequence Synthetic oligonucleotide
gcgcgctcgctcgctcacc

SEQ ID NO: 158 - DNA, 22 bp, Artificial Sequence
  Description of Artificial Sequence Synthetic oligonucleotide
ctaggtgagcgagcgagcgcgc

SEQ ID NO: 159 - DNA, 75 bp, Artificial Sequence
  Description of Artificial Sequence Synthetic oligonucleotide
cctgcaggactgaggccgcccgggcaaagcccgggcgtcgggcgacctttggtcgcccggcctcagtcctgcagg

SEQ ID NO: 160 - DNA, 130 bp, Artificial Sequence
  Description of Artificial Sequence Synthetic polynucleotide
aggaacccctagtgatggagttggccactccctctctgcgcgctcgctcgctcactgagtgcgggcgaccaaaggtcgcccgacgcccgggcgcactcagtgagcgagcgagcgcgcagctgcctgcagg

SEQ ID NO: 161 - DNA, 142 bp, Artificial Sequence
  Description of Artificial Sequence Synthetic polynucleotide
cctgcaggcagctgcgcgctcgctcgctccctaggactgaggccgcccgggcgtcgggcgacctttggtcgcccggcctcagtcctagggagcgagcgagcgcgcagagagggagtggccaactccatcactaggggttcct

SEQ ID NO: 162 - DNA, 80 bp, Artificial Sequence
  Description of Artificial Sequence Synthetic oligonucleotide
gcgcgctcgctcgctcactgagtgcgggcgaccaaaggtcgcccgacgcccgggcgcactcagtgagcgagcgagcgcgc

SEQ ID NO: 163 - DNA, 21 bp, Artificial Sequence
  Description of Artificial Sequence Synthetic oligonucleotide
gcgcgctcgctcgctcactga

SEQ ID NO: 164 - DNA, 18 bp, Artificial Sequence
  Description of Artificial Sequence Synthetic oligonucleotide
gtgagcgagcgagcgcgc

SEQ ID NO: 165 - DNA, 89 bp, Artificial Sequence
  Description of Artificial Sequence Synthetic oligonucleotide
gcgcgctcgctcgctcactgaggccgcccgggcaaagcccgggcgtcgggcgactttgtcgcccggcctcagtgagcgagcgagcgcgc

SEQ ID NO: 166 - DNA, 89 bp, Artificial Sequence
  Description of Artificial Sequence Synthetic oligonucleotide
gcgcgctcgctcgctcactgaggccgggcgacaaagtcgcccgacgcccgggctttgcccgggcggcctcagtgagcgagcgagcgcgc

SEQ ID NO: 167 - DNA, 87 bp, Artificial Sequence
  Description of Artificial Sequence Synthetic oligonucleotide
gcgcgctcgctcgctcactgaggccgcccgggcaaagcccgggcgtcgggcgattttcgcccggcctcagtgagcgagcgagcgcgc

SEQ ID NO: 168 - DNA, 87 bp, Artificial Sequence
  Description of Artificial Sequence Synthetic oligonucleotide
gcgcgctcgctcgctcactgaggccgggcgaaaatcgcccgacgcccgggctttgcccgggcggcctcagtgagcgagcgagcgcgc

SEQ ID NO: 169 - DNA, 85 bp, Artificial Sequence
  Description of Artificial Sequence Synthetic oligonucleotide
gcgcgctcgctcgctcactgaggccgcccgggcaaagcccgggcgtcgggcgtttcgcccggcctcagtgagcgagcgagcgcgc

SEQ ID NO: 170 - DNA, 85 bp, Artificial Sequence
  Description of Artificial Sequence Synthetic oligonucleotide
gcgcgctcgctcgctcactgaggccgggcgaaacgcccgacgcccgggctttgcccgggcggcctcagtgagcgagcgagcgcgc

SEQ ID NO: 171 - DNA, 89 bp, Artificial Sequence
  Description of Artificial Sequence Synthetic oligonucleotide
gcgcgctcgctcgctcactgaggccgcccgggaaacccgggcgtcgggcgacctttggtcgcccggcctcagtgagcgagcgagcgcgc

SEQ ID NO: 172 - DNA, 89 bp, Artificial Sequence
  Description of Artificial Sequence Synthetic oligonucleotide
gcgcgctcgctcgctcactgaggccgggcgaccaaaggtcgcccgacgcccgggtttcccgggcggcctcagtgagcgagcgagcgcgc

SEQ ID NO: 173 - DNA, 87 bp, Artificial Sequence
  Description of Artificial Sequence Synthetic oligonucleotide
gcgcgctcgctcgctcactgaggccgcccggaaaccgggcgtcgggcgacctttggtcgcccggcctcagtgagcgagcgagcgcgc

SEQ ID NO: 174 - DNA, 87 bp, Artificial Sequence
  Description of Artificial Sequence Synthetic oligonucleotide
gcgcgctcgctcgctcactgaggccgggcgaccaaaggtcgcccgacgcccggtttccgggcggcctcagtgagcgagcgagcgcgc

SEQ ID NO: 175 - DNA, 85 bp, Artificial Sequence
  Description of Artificial Sequence Synthetic oligonucleotide
gcgcgctcgctcgctcactgaggccgcccgaaacgggcgtcgggcgacctttggtcgcccggcctcagtgagcgagcgagcgcgc

SEQ ID NO: 176 - DNA, 85 bp, Artificial Sequence
  Description of Artificial Sequence Synthetic oligonucleotide
gcgcgctcgctcgctcactgaggccgggcgaccaaaggtcgcccgacgcccgtttcgggcggcctcagtgagcgagcgagcgcgc

SEQ ID NO: 177 - DNA, 83 bp, Artificial Sequence
  Description of Artificial Sequence Synthetic oligonucleotide
gcgcgctcgctcgctcactgaggccgcccaaagggcgtcgggcgacctttggtcgcccggcctcagtgagcgagcgagcgcgc

SEQ ID NO: 178 - DNA, 83 bp, Artificial Sequence
  Description of Artificial Sequence Synthetic oligonucleotide
gcgcgctcgctcgctcactgaggccgggcgaccaaaggtcgcccgacgccctttgggcggcctcagtgagcgagcgagcgcgc

SEQ ID NO: 179 - DNA, 81 bp, Artificial Sequence
  Description of Artificial Sequence Synthetic oligonucleotide
gcgcgctcgctcgctcactgaggccgccaaaggcgtcgggcgacctttggtcgcccggcctcagtgagcgagcgagcgcgc

SEQ ID NO: 180 - DNA, 81 bp, Artificial Sequence
  Description of Artificial Sequence Synthetic oligonucleotide
gcgcgctcgctcgctcactgaggccgggcgaccaaaggtcgcccgacgcctttggcggcctcagtgagcgagcgagcgcgc

SEQ ID NO: 181 - DNA, 79 bp, Artificial Sequence
  Description of Artificial Sequence Synthetic oligonucleotide
gcgcgctcgctcgctcactgaggccgcaaagcgtcgggcgacctttggtcgcccggcctcagtgagcgagcgagcgcgc

SEQ ID NO: 182 - DNA, 79 bp, Artificial Sequence
  Description of Artificial Sequence Synthetic oligonucleotide
gcgcgctcgctcgctcactgaggccgggcgaccaaaggtcgcccgacgctttgcggcctcagtgagcgagcgagcgcgc

SEQ ID NO: 183 - DNA, 81 bp, Artificial Sequence
  Description of Artificial Sequence Synthetic oligonucleotide
ctgcgcgctcgctcgctcactgaggccgaaacgtcgggcgacctttggtcgcccggcctcagtgagcgagcgagcgcgcag

SEQ ID NO: 184 - DNA, 81 bp, Artificial Sequence
  Description of Artificial Sequence Synthetic oligonucleotide
ctgcgcgctcgctcgctcactgaggccgggcgaccaaaggtcgcccgacgtttcggcctcagtgagcgagcgagcgcgcag

SEQ ID NO: 185 - DNA, 72 bp, Artificial Sequence; identical to SEQ ID NO: 104
  Description of Artificial Sequence Synthetic oligonucleotide

SEQ ID NO: 186 - DNA, 80 bp, Artificial Sequence; identical to SEQ ID NO: 114
  Description of Artificial Sequence Synthetic oligonucleotide

SEQ ID NO: 187 - DNA, 79 bp, Artificial Sequence; identical to SEQ ID NO: 115
  Description of Artificial Sequence Synthetic oligonucleotide

SEQ ID NO: 188 - DNA, 48 bp, Artificial Sequence
  Description of Artificial Sequence Synthetic oligonucleotide
ggagtcaaagttctgtttgccctgatctgcatcgctgtggccgaggcc

SEQ ID NO: 189 - DNA, 99 bp, Artificial Sequence
  Description of Artificial Sequence Synthetic oligonucleotide
attcataccaacttgaagaaaaagttcagcctcttcatcctggtctttctcctgttcgcagtcatctgtgtttggaagaaagggagcgactatgaggcc

SEQ ID NO: 190 - DNA, 588 bp, Artificial Sequence
  Description of Artificial Sequence Synthetic polynucleotide
gcccctctccctcccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaaacgtctaggccccccgaaccacggggacgtggttttcctttgaaaaacacgatgataatatggccacaacc